SEQUENCE LISTING (212 sequences)

SEQ ID NO: 1 - protein, 11 aa, synthetic construct
PPPALPPKRRR

SEQ ID NO: 2 - protein, 32 aa, synthetic construct
LVGALMHVMQKRSRAIHSSDEGEDQAGDEDED

SEQ ID NO: 3 - protein, 43 aa, synthetic construct
ITIRAAFLEKENTALRTEIAELEKEVGRCENIVSKYETRYGPL

SEQ ID NO: 4 - protein, 43 aa, synthetic construct
LEIRAAFLEKENTALRTRAAELRKRVGRCRNIVSKYETRYGPL

SEQ ID NO: 5 - protein, 70 aa, synthetic construct
SYYHHHHHHLESTSLYKKAGSGSNLVAQLENEVASLENENETLKKKNLHKKDLIAYLEKEIANLRKKIEE

SEQ ID NO: 6 - protein, 73 aa, synthetic construct
SYYHHHHHHLESTSLYKKAGSGSARNAYLRKKIARLKKDNLQLERDEQNLEKIIANLRDEIARLENEVASHEQ

SEQ ID NO: 7 - protein, 65 aa, synthetic construct
SYYHHHHHHLESTSLYKKAGSGSNEVTTLENDAAFIENENAYLEKEIARLRKEKAALRNRLAHKK

SEQ ID NO: 8 - protein, 77 aa, synthetic construct
SYYHHHHHHLESTSLYKKAGSGSQKVAELKNRVAVKLNRNEQLKNKVEELKNRNAYLKNELATLENEVARLENDVAE

SEQ ID NO: 9 - protein, 64 aa, synthetic construct
ENLYFQGENLYFQGDSSESCWNCGRKASETCSGCNTARYCGSFCQHKDWEKHHHICGQTLQAQQ

SEQ ID NO: 10 - protein, 145 aa, synthetic construct
MAVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRP

SEQ ID NO: 11 - protein, 88 aa, synthetic construct
GPLGSPLTASMLASAPPQEQKQMLGERLFPLIQAMHPTLAGKITGMLLEIDNSELLHMLESPESLRSKVDEAVAVLQAHQAKEAAQKA

SEQ ID NO: 12 - protein, 107 aa, synthetic construct
NTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVV

SEQ ID NO: 13 - protein, 65 aa, synthetic construct
SYYHHHHHHLESTSLYKKAGSGSNLLATLRSTAAVLENENHVLEKEKEKLRKEKEQLLNKLEAYK

SEQ ID NO: 14 - protein, 73 aa, synthetic construct
SYYHHHHHHLESTSLYKKAGSGSKRIAYLRKKIAALKKDNANLEKDIANLENEIERLIKEIKTLENEVASHEQ

SEQ ID NO: 15 - protein, 62 aa, synthetic construct [sequence omitted]

SEQ ID NO: 16 - protein, 85 aa, synthetic construct
ESDSVEFNNAISYVNKIKTRFLDHPEIYRSFLEILHTYQKEQLHTKGRPFRGMSEEEVFTEVANLFRGQEDLLSEFGQFLPEAKR

SEQ ID NO: 17 - protein, 36 aa, synthetic construct
LGPLPPGWEVRSTVSGRIYFVDHNNRTTQFTDPRLH

SEQ ID NO: 18 - protein, 37 aa, synthetic construct
GAMGPLPPGWEKRTDSNGRVYFVNHNTRITQWEDPRS

SEQ ID NO: 19 - protein, 101 aa, synthetic construct
SYYHHHHHHLESTSLYKKAGSEFFRRERNKMAAAKCRNRRRELTDTLQAETDQLEDEKSALQTEIANLLKEKEKLEFILAAHRPACKIPDDLGFPEEMSLE

SEQ ID NO: 20 - protein, 59 aa, synthetic construct
SYYHHHHHHLESTSLYKKAGSGSQKVESLKQKIEELKQRKAQLKNDIANLEKEIAYAET

SEQ ID NO: 21 - protein, 147 aa, synthetic construct
MEPAMEPETLEARINRATNPLNKELDWASINGFCEQLNEDFEGPPLATRLLAHKIQSPQEWEAIQALTVLETCMKSCGKRFHDEVGKFRFLNELIKVVSPKYLGSRTSEKVKNKILELLYSWTVGLPEEVKIAEAYQMLKKQGIVKS

SEQ ID NO: 22 - protein, 171 aa, synthetic construct
GAMGSMAEAEGESLESWLNKATNPSNRQEDWEYIIGFCDQINKELEGPQIAVRLLAHKIQSPQEWEALQALTVLEACMKNCGRRFHNEVGKFRFLNELIKVVSPKYLGDRVSEKVKTKVIELLYSWTMALPEEAKIKDAYHMLKRQGIVQSDPPIPVDRTLIPSPPPRPKN

SEQ ID NO: 23 - protein, 67 aa, synthetic construct
SYYHHHHHHLESTSLYKKAGSGSQKVEELKNKIAELENRNAVKKNRVAHLKQEIAYLKDELAAHEFE

SEQ ID NO: 24 - protein, 64 aa, synthetic construct
SYYHHHHHHLESTSLYKKAGSGSFENVTHEFILATLENENAKLRRLEAKLERELARLRNEVAWL

SEQ ID NO: 25 - protein, 168 aa, synthetic construct
AMADLEQKVLEMEASTYDGVFIWKISDFPRKRQEAVAGRIPAIFSPAFYTSRYGYKMCLRIYLNGDGTGRGTHLSLFFVVMKGPNDALLRWPFNQKVTLMLLDQNNREHVIDAFRPDVTSSSFQRPVNDMNIASGCPLFCPVSKMEAKNSYVRDDAIFIKAIVDLTGL

SEQ ID NO: 26 - protein, 159 aa, synthetic construct
ASIKLQSSDGEIFEVDVEIAKQSVTIKTMLEDLGMDDEGDDDPVPLPNVNAAILKKVIQWCTHHKDDPPPPEDDENKEKRTDDIPVWDQEFLKVDQGTLFELILAANYLDIKGLLDVTCKTVANMIKGKTPEEIRKTFNIKNDFTEEEEAQVRKENQWC

SEQ ID NO: 27 - protein, 67 aa, synthetic construct
SYYHHHHHHLESTSLYKKAGSGSNTVKELKNYIQELEERNAELKNLKEHLKFAKAELEFELAAHKFE

SEQ ID NO: 28 - protein, 77 aa, synthetic construct
SYYHHHHHHLESTSLYKKAGSGSQKVAQLKNRVAYKLKENAKLENIVARLENDNANLEKDIANLEKDIANLERDVAR

SEQ ID NO: 29 - protein, 81 aa, synthetic construct
LCTMKKGPSGYGFNLHSDKSKPGQFIRSVDPDSPAEASGLRAQDRIVEVNGVCMEGKQHGDVVSAIRAGGDETKLLVVDRE

SEQ ID NO: 30 - protein, 97 aa, synthetic construct
SSGAIIYTVELKRYGGPLGITISGTEEPFDPIIISSLTKGGLAERTGAIHIGDRILAINSSSLKGKPLSEAIHLLQMAGETVTLKIKKQTDAQPASS

SEQ ID NO: 31 - protein, 109 aa, synthetic construct
GNNLETYEWYNKSISRDKAEKLLLDTGKEGAFMVRDSRTPGTYTVSVFTKAIISENPCIKHYHIKETNDSPKRYYVAEKYVFDSIPLLIQYHQYNGGGLVTRLRYPVCG

SEQ ID NO: 32 - protein, 104 aa, synthetic construct
GSHPWFFGKIPRAKAEEMLSKQRHDGAFLIRESESAPGDFSLSVKFGNDVQHFKVLRDGAGKYFLWVVKFNSLNELVDYHRSTSVSRNQQIFLRDIEQVPQQPT

SEQ ID NO: 33 - protein, 152 aa, synthetic construct [sequence omitted]

SEQ ID NO: 34 - protein, 57 aa, synthetic construct
AEYVRALFDFNGNDEEDLPFKKGDILRIRDKPEEQWWNAEDSEGKRGMIPVPYVEKY

SEQ ID NO: 35 - protein, 60 aa, synthetic construct
LIKHMRAEALFDFTGNSKLELNFKAGDVIFLLSRINKDWLEGTVRGATGIFPLSFVKILK

SEQ ID NO: 36 - protein, 153 aa, synthetic construct
GSHMRLGAQSIQPTANLDRTDDLVYLNVMELVRAVLELKNELAQLPPEGYVVVVKNVGLTLRKLIGSVDDLLPSLPSSSRTEIEGTQKLLNKDLAELINKMRLAQQNAVTSLSEECKRQMLTASHTLAVDAKNLLDAVDQAKVLANLAHPPAE

SEQ ID NO: 37 - protein, 70 aa, synthetic construct
GAMATPGSENVLPREPLIATAVKFLQNSRVRQSPLATRRAFLKKKGLTDEEIDMAFQQSGTAADEPSSLW

SEQ ID NO: 38 - protein, 20 aa, synthetic construct
RPPTISNPPPLISSAKHPSV

SEQ ID NO: 39 - protein, 20 aa, synthetic construct
NFLQSRPEPTAPPEESFRSG

SEQ ID NO: 40 - protein, 20 aa, synthetic construct
SKGTGLNPNAKVWQEIAPGN

SEQ ID NO: 41 - protein, 20 aa, synthetic construct
PDGGTTFEHLWSSLEPDSTY

SEQ ID NO: 42 - protein, 20 aa, synthetic construct
PATSQHPPPPPGHRSQAPSH

SEQ ID NO: 43 - protein, 20 aa, synthetic construct
ELNSLLILLEAAEYLERRDR

SEQ ID NO: 44 - protein, 20 aa, synthetic construct
FQMPADTPPPAYLPPEDPMT

SEQ ID NO: 45 - protein, 20 aa, synthetic construct
ERESNEEPPPPYEDPYWGNG

SEQ ID NO: 46 - protein, 20 aa, synthetic construct
VSSTKLVSFHDDSDEDLLHI

SEQ ID NO: 47 - protein, 20 aa, synthetic construct
AAATPISTFHDDSDEDLLHV

SEQ ID NO: 48 - protein, 20 aa, synthetic construct
HDDSLPHPQQATDDSGHESD

SEQ ID NO: 49 - protein, 20 aa, synthetic construct
GSPNAGSVEQTPKKPGLRRR

SEQ ID NO: 50 - protein, 20 aa, synthetic construct
TDEEREETEEEVYLLNSTTL

SEQ ID NO: 51 - protein, 20 aa, synthetic construct
DGNVSGTQRLDSATVRTYSC

SEQ ID NO: 52 - protein, 20 aa, synthetic construct
ALVDDAADYEPPPSNNEEAL

SEQ ID NO: 53 - protein, 20 aa, synthetic construct
RELFDDPSYVNVQNLDKARQ

SEQ ID NO: 54 - protein, 20 aa, synthetic construct
KNTKSMNFDNPVYRKTTEEE

SEQ ID NO: 55 - protein, 20 aa, synthetic construct
RSLPSTWIENKLYGMSDPNW

SEQ ID NO: 56 - protein, 20 aa, synthetic construct
VVDNSPPPALPPKKRQSAPS

SEQ ID NO: 57 - protein, 20 aa, synthetic construct
TQRSKPQPAVPPRPSADLIL

SEQ ID NO: 58 - protein, 20 aa, synthetic construct
SATRELDELMASLSDFKIQG

SEQ ID NO: 59 - protein, 20 aa, synthetic construct
DLALSENWAQEFLAAGDAVD

SEQ ID NO: 60 - protein, 16 aa, synthetic construct
GSGSGSGSGSGSGSGS

SEQ ID NO: 61 - protein, 20 aa, synthetic construct
GSGSGSGSGSGSGSGSGSGS

SEQ ID NO: 62 - protein, 30 aa, synthetic construct
GSGSGSGSGSGSGSGSGSGSGSGSGSGSGS

SEQ ID NO: 63 - protein, 15 aa, synthetic construct
GGGGSGGGGSGGGGS

SEQ ID NO: 64 - protein, 20 aa, synthetic construct
GGGGSGGGGSGGGGSGGGGS

SEQ ID NO: 65 - protein, 10 aa, synthetic construct
EAAAKEAAAK

SEQ ID NO: 66 - protein, 15 aa, synthetic construct
EAAAKEAAAKEAAAK

SEQ ID NO: 67 - protein, 20 aa, synthetic construct
EAAAKEAAAKEAAAKEAAAK

SEQ ID NO: 68 - protein, 6 aa, synthetic construct
AAAGGM

SEQ ID NO: 69 - protein, 14 aa, synthetic construct
AAAGGMPPAAAGGM

SEQ ID NO: 70 - protein, 6 aa, synthetic construct
AAAGGM

SEQ ID NO: 71 - protein, 9 aa, synthetic construct
PPAAAGGMM

SEQ ID NO: 72 - protein, 12 aa, synthetic construct
GSAGSAAGSGEF

SEQ ID NO: 73 - protein, 18 aa, synthetic construct
KLSGGGGSGGGGSGGGGS

SEQ ID NO: 74 - protein, 40 aa, synthetic construct
GSAGSAAGSGEFGSAEAAAKEAAAKAGSAGSAAGSGEFGS

SEQ ID NO: 75 - protein, 36 aa, synthetic construct
GSAGSAAGSGEFAEAAAKEAAAKAGSAGSAAGSGEF

SEQ ID NO: 76 - protein, 50 aa, synthetic construct
GSAGSAAGSGEFGSAEAAAKEAAAKEAAAKEAAAKAGSAGSAAGSGEFGS

SEQ ID NO: 77 - protein, 211 aa, synthetic construct
AEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSA

SEQ ID NO: 78 - protein, 16 aa, synthetic construct
GGGGSGGGGSGGGGAS

SEQ ID NO: 79 - protein, 36 aa, synthetic construct
GSAGSAAGSGEFGSAGSAAGSGEFGSAGSAAGSGEF

SEQ ID NO: 80 - protein, 116 aa, synthetic construct
GSAGSAAGSGEFGSAEAAAKEAAAKEAAAKEAAAKAGSAGSAAGSGEFGSEQKLISEEDLEQKLISEEDLEQKLISEEDLGSAGSAAGSGEFGSAGSAAGSGEFGSAGSAAGSGEF

SEQ ID NO: 81 - protein, 110 aa, synthetic construct
GSAGSAAGSGEFGSAEAAAKEAAAKEAAAKEAAAKAGSAGSAAGSGEFGSDYKDDDDKDYKDDDDKDYKDDDDKGSAGSAAGSGEFGSAGSAAGSGEFGSAGSAAGSGEF

SEQ ID NO: 82 - DNA, 30 bp, synthetic construct
  source 1..30: mol_type = other DNA
cggcacccctacaaacagaaggaatataaa

SEQ ID NO: 83 - protein, 1101 aa, Homo sapiens
MSAKAISEQTGKELLYKFICTTSAIQNRFKYARVTPDTDWARLLQDHPWLLSQNLVVKPDQLIKRRGKLGLVGVNLTLDGVKSWLKPRLGQEATVGKATGFLKNFLIEPFVPHSQAEEFYVCIYATREGDYVLFHHEGGVDVGDVDAKAQKLLVGVDEKLNPEDIKKHLLVHAPEDKKEILASFISGLFNFYEDLYFTYLEINPLVVTKDGVYVLDLAAKVDATADYICKVKWGDIEFPPPFGREAYPEEAYIADLDAKSGASLKLTLLNPKGRIWTMVAGGGASVVYSDTICDLGGVNELANYGEYSGAPSEQQTYDYAKTILSLMTREKHPDGKILIIGGSIANFTNVAATFKGIVRAIRDYQGPLKEHEVTIFVRRGGPNYQEGLRVMGEVGKTTGIPIHVFGTETHMTAIVGMALGHRPIPNQPPTAAHTANFLLNASGSTSTPAPSRTASFSESRADEVAPAKKAKPAMPQDSVPSPRSLQGKSTTLFSRHTKAIVWGMQTRAVQGMLDFDYVCSRDEPSVAAMVYPFTGDHKQKFYWGHKEILIPVFKNMADAMRKHPEVDVLINFASLRSAYDSTMETMNYAQIRTIAIIAEGIPEALTRKLIKKADQKGVTIIGPATVGGIKPGCFKIGNTGGMLDNILASKLYRPGSVAYVSRSGGMSNELNNIISRTTDGVYEGVAIGGDRYPGSTFMDHVLRYQDTPGVKMIVVLGEIGGTEEYKICRGIKEGRLTKPIVCWCIGTCATMFSSEVQFGHAGACANQASETAVAKNQALKEAGVFVPRSFDELGEIIQSVYEDLVANGVIVPAQEVPPPTVPMDYSWARELGLIRKPASFMTSICDERGQELIYAGMPITEVFKEEMGIGGVLGLLWFQKRLPKYSCQFIEMCLMVTADHGPAVSGAHNTIICARAGKDLVSSLTSGLLTIGDRFGGALDAAAKMFSKAFDSGIIPMEFVNKMKKEGKLIMGIGHRVKSINNPDMRVQILKDYVRQHFPATPLLDYALEVEKITTSKKPNLILNVDGLIGVAFVDMLRNCGSFTREEADEYIDIGALNGIFVLGRSMGFIGHYLDQKRLKQGLYRHPWDDISYVLPEHMSM

SEQ ID NO: 84 - protein, 394 aa, Escherichia coli
MKNCVIVSAVRTAIGSFNGSLASTSAIDLGATVIKAAIERAKIDSQHVDEVIMGNVLQAGLGQNPARQALLKSGLAETVCGFTVNKVCGSGLKSVALAAQAIQAGQAQSIVAGGMENMSLAPYLLDAKARSGYRLGDGQVYDVILRDGLMCATHGYHMGITAENVAKEYGITREMQDELALHSQRKAAAAIESGAFTAEIVPVNVVTRKKTFVFSQDEFPKANSTAEALGALRPAFDKAGTVTAGNASGINDGAAALVIMEESAALAAGLTPLARIKSYASGGVPPALMGMGPVPATQKALQLAGLQLADIDLIEANEAFAAQFLAVGKNLGFDSEKVNVNGGAIALGHPIGASGARILVTLLHAMQARDKTLGLATLCIGGGQGIAMVIERLN

SEQ ID NO: 85 - protein, 282 aa, Clostridium acetobutylicum
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKRPDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSK

SEQ ID NO: 86 - protein, 261 aa, Clostridium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 87 - protein, 394 aa, Cupriavidus necator
MTREVVVVSGVRTAIGTFGGSLKDVAPAELGALVVREALARAQVSGDDVGHVVFGNVIQTEPRDMYLGRVAAVNGGVTINAPALTVNRLCGSGLQAIVSAAQTILLGDTDVAIGGGAESMSRAPYLAPAARWGARMGDAGLVDMMLGALHDPFHRIHMGVTAENVAKEYDISRAQQDEAALESHRRASAAIKAGYFKDQIVPVVSKGRKGDVTFDTDEHVRHDATIDDMTKLRPVFVKENGTVTAGNASGLNDAAAAVVMMERAEAERRGLKPLARLVSYGHAGVDPKAMGIGPVPATKIALERAGLQVSDLDVIEANEAFAAQACAVTKALGLDPAKVNPNGSGISLGHPIGATGALITVKALHELNRVQGRYALVTMCIGGGQGIAAIFERI

SEQ ID NO: 88 - protein, 397 aa, Treponema denticola
MIVKPMVRNNICLNAHPQGCKKGVEDQIEYTKKRITAEVKAGAKAPKNVLVLGCSNGYGLASRITAAFGYGAATIGVSFEKAGSETKYGTPGWYNNLAFDEAAKREGLYSVTIDGDAFSDEIKAQVIEEAKKKGIKFDLIVYSLASPVRTDPDTGIMHKSVLKPFGKTFTGKTVDPFTGELKEISAEPANDEEAAATVKVMGGEDWERWIKQLSKEGLLEEGCITLAYSYIGPEATQALYRKGTIGKAKEHLEATAHRLNKENPSIRAFVSVNKGLVTRASAVIPVIPLYLASLFKVMKEKGNHEGCIEQITRLYAERLYRKDGTIPVDEENRIRIDDWELEEDVQKAVSALMEKVTGENAESLTDLAGYRHDFLASNGFDVEGINYEAEVERFDRI

SEQ ID NO: 89 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 90 - protein, 491 aa, Saccharomyces cerevisiae
MKLSTKLCWCGIKGRLRPQKQQQLHNTNLQMTELKKQKTAEQKTRPQNVGIKGIQIYIPTQCVNQSELEKFDGVSQGKYTIGLGQTNMSFVNDREDIYSMSLTVLSKLIKSYNIDTNKIGRLEVGTETLIDKSKSVKSVLMQLFGENTDVEGIDTLNACYGGTNALFNSLNWIESNAWDGRDAIVVCGDIAIYDKGAARPTGGAGTVAMWIGPDAPIVFDSVRASYMEHAYDFYKPDFTSEYPYVDGHFSLTCYVKALDQVYKSYSKKAISKGLVSDPAGSDALNVLKYFDYNVFHVPTCKLVTKSYGRLLYNDFRANPQLFPEVDAELATRDYDESLTDKNIEKTFVNVAKPFHKERVAQSLIVPTNTGNMYTASVYAAFASLLNYVGSDDLQGKRVGLFSYGSGLAASLYSCKIVGDVQHIIKELDITNKLAKRITETPKDYEAAIELRENAHLKKNFKPQGSIEHLQSGVYYLTNIDDKFRRSYDVKK

SEQ ID NO: 91 - protein, 381 aa, Saccharomyces cerevisiae
MVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNR

SEQ ID NO: 92 - protein, 443 aa, Saccharomyces cerevisiae
MSLPFLTSAPGKVIIFGEHSAVYNKPAVAASVSALRTYLLISESSAPDTIELDFPDISFNHKWSINDFNAITEDQVNSQKLAKAQQATDGLSQELVSLLDPLLAQLSESFHYHAAFCFLYMFVCLCPHAKNIKFSLKSTLPIGAGLGSSASISVSLALAMAYLGGLIGSNDLEKLSENDKHIVNQWAFIGEKCIHGTPSGIDNAVATYGNALLFEKDSHNGTINTNNFKFLDDFPAIPMILTYTRIPRSTKDLVARVRVLVTEKFPEVMKPILDAMGECALQGLEIMTKLSKCKGTDDEAVETNNELYEQLLELIRINHGLLVSIGVSHPGLELIKNLSDDLRIGSTKLTGAGGGGCSLTLLRRDITQEQIDSFKKKLQDDFSYETFETDLGGTGCCLLSAKNLNKDLKIKSLVFQLFENKTTTKQQIDDLLLPGNTNLPWTS

SEQ ID NO: 93 - protein, 452 aa, Saccharomyces cerevisiae
MSELRAFSAPGKALLAGGYLVLDTKYEAFVVGLSARMHAVAHPYGSLQGSDKFEVRVKSKQFKDGEWLYHISPKSGFIPVSIGGSKNPFIEKVIANVFSYFKPNMDDYCNRNLFVIDIFSDDAYHSQEDSVTEHRGNRRLSFHSHRIEEVPKTGLGSSAGGLVTVLTTALASFFVSDLENNVDKYREVIHNLAQVAHCQAQGKIGSGFDVAAAAYGSIRYRRFPPALISNLPDIGSATYGSKLAHLVDEEDWNITIKSNHLPSGLTLWMGDIKNGSETVKLVQKVKNWYDSHMPESLKIYTELDHANSRFMDGLSKLDRLHETHDDYSDQIFESLERNDCTCQKYPEITEVRDAVATIRRSFRKITKESGADIEPPVQTSLLDDCQTLKGVLTCLIPGAGGYDAIAVITKQDVDLRAQTANDKRFSKVQWLDVTQADWGVRKEKDPETYLDK

SEQ ID NO: 94 - protein, 396 aa, Saccharomyces cerevisiae
MTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKE

SEQ ID NO: 95 - protein, 288 aa, Saccharomyces cerevisiae
MTADNNSMPHGAVSSYAKLVQNQTPEDILEEFPEIIPLQQRPNTRSSETSNDESGETCFSGHDEEQIKLMNENCIVLDWDDNAIGAGTKKVCHLMENIEKGLLHRAFSVFIFNEQGELLLQQRATEKITFPDLWTNTCCSHPLCIDDELGLKGKLDDKIKGAITAAVRKLDHELGIPEDETKTRGKFHFLNRIHYMAPSNEPWGEHEIDYILFYKINAKENLTVNPNVNEVRDFKWVSPNDLKTMFADPSYKFTPWFKIICENYLFNWWEQLDDLSEVENDRQIHRML

SEQ ID NO: 96 - protein, 335 aa, Saccharomyces cerevisiae
MEAKIDELINNDPVWSSQNESLISKPYNHILLKPGKNFRLNLIVQINRVMNLPKDQLAIVSQIVELLHNSSLLIDDIEDNAPLRRGQTTSHLIWGVPSTINTANYMYFRAMQLVSQLTTKEPLYHWLITIFNEELINLHRGQGLDIYWRDFLPEIIPTQEMYLNMVMNKTGGLFRLTLRLMEALSPSSHHGHSLVPFINLLGIIYQIRDDYLNLKDFQMSSEKGFAEDITEGKLSFPIVHALNFTKTKGQTEQHNEILRILLLRTSDKDIKLKLIQILEFDTNSLAYTKNFINQLVNMIKNDNENKYLPDLASHSDTATNLHDELLYIIDHLSEL

SEQ ID NO: 97 - protein, 2233 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLSDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVSVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 98 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 99 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 100 - protein, 395 aa, Cannabis sativa
MGLSSVCTFSFQTNYHTLLNPHNNNPKTSLLCYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 101 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 102 - protein, 545 aa, Cannabis sativa
MNCSTFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAACKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 103 - protein, 1529 aa, synthetic construct
MSAKAISEQTGKELLYKFICTTSAIQNRFKYARVTPDTDWARLLQDHPWLLSQNLVVKPDQLIKRRGKLGLVGVNLTLDGVKSWLKPRLGQEATVGKATGFLKNFLIEPFVPHSQAEEFYVCIYATREGDYVLFHHEGGVDVGDVDAKAQKLLVGVDEKLNPEDIKKHLLVHAPEDKKEILASFISGLFNFYEDLYFTYLEINPLVVTKDGVYVLDLAAKVDATADYICKVKWGDIEFPPPFGREAYPEEAYIADLDAKSGASLKLTLLNPKGRIWTMVAGGGASVVYSDTICDLGGVNELANYGEYSGAPSEQQTYDYAKTILSLMTREKHPDGKILIIGGSIANFTNVAATFKGIVRAIRDYQGPLKEHEVTIFVRRGGPNYQEGLRVMGEVGKTTGIPIHVFGTETHMTAIVGMALGHRPIPNQPPTAAHTANFLLNASGSTSTPAPSRTASFSESRADEVAPAKKAKPAMPQDSVPSPRSLQGKSTTLFSRHTKAIVWGMQTRAVQGMLDFDYVCSRDEPSVAAMVYPFTGDHKQKFYWGHKEILIPVFKNMADAMRKHPEVDVLINFASLRSAYDSTMETMNYAQIRTIAIIAEGIPEALTRKLIKKADQKGVTIIGPATVGGIKPGCFKIGNTGGMLDNILASKLYRPGSVAYVSRSGGMSNELNNIISRTTDGVYEGVAIGGDRYPGSTFMDHVLRYQDTPGVKMIVVLGEIGGTEEYKICRGIKEGRLTKPIVCWCIGTCATMFSSEVQFGHAGACANQASETAVAKNQALKEAGVFVPRSFDELGEIIQSVYEDLVANGVIVPAQEVPPPTVPMDYSWARELGLIRKPASFMTSICDERGQELIYAGMPITEVFKEEMGIGGVLGLLWFQKRLPKYSCQFIEMCLMVTADHGPAVSGAHNTIICARAGKDLVSSLTSGLLTIGDRFGGALDAAAKMFSKAFDSGIIPMEFVNKMKKEGKLIMGIGHRVKSINNPDMRVQILKDYVRQHFPATPLLDYALEVEKITTSKKPNLILNVDGLIGVAFVDMLRNCGSFTREEADEYIDIGALNGIFVLGRSMGFIGHYLDQKRLKQGLYRHPWDDISYVLPEHMSMKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASSYYHHHHHHLESTSLYKKAGSGSNLVAQLENEVASLENENETLKKKNLHKKDLIAYLEKEIANLRKKIEEGSAGSAAGSGEFGSAEAAAKEAAAKAGSAGSAAGSGEFGSSYYHHHHHHLESTSLYKKAGSGSARNAYLRKKIARLKKDNLQLERDEQNLEKIIANLRDEIARLENEVASHEQ

SEQ ID NO: 104 - protein, 821 aa, synthetic construct
MKNCVIVSAVRTAIGSFNGSLASTSAIDLGATVIKAAIERAKIDSQHVDEVIMGNVLQAGLGQNPARQALLKSGLAETVCGFTVNKVCGSGLKSVALAAQAIQAGQAQSIVAGGMENMSLAPYLLDAKARSGYRLGDGQVYDVILRDGLMCATHGYHMGITAENVAKEYGITREMQDELALHSQRKAAAAIESGAFTAEIVPVNVVTRKKTFVFSQDEFPKANSTAEALGALRPAFDKAGTVTAGNASGINDGAAALVIMEESAALAAGLTPLARIKSYASGGVPPALMGMGPVPATQKALQLAGLQLADIDLIEANEAFAAQFLAVGKNLGFDSEKVNVNGGAIALGHPIGASGARILVTLLHAMQARDKTLGLATLCIGGGQGIAMVIERLNKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASSYYHHHHHHLESTSLYKKAGSGSNEVTTLENDAAFIENENAYLEKEIARLRKEKAALRNRLAHKKGSAGSAAGSGEFGSAEAAAKEAAAKAGSAGSAAGSGEFGSSYYHHHHHHLESTSLYKKAGSGSQKVAELKNRVAVKLNRNEQLKNKVEELKNRNAYLKNELATLENEVARLENDVAE

SEQ ID NO: 105 - protein, 776 aa, synthetic construct
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKRPDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSKKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASENLYFQGENLYFQGDSSESCWNCGRKASETCSGCNTARYCGSFCQHKDWEKHHHICGQTLQAQQGSAGSAAGSGEFGSAEAAAKEAAAKAGSAGSAAGSGEFGSMAVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRP

SEQ ID NO: 106 - protein, 741 aa, synthetic construct
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNRKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASGPLGSPLTASMLASAPPQEQKQMLGERLFPLIQAMHPTLAGKITGMLLEIDNSELLHMLESPESLRSKVDEAVAVLQAHQAKEAAQKAGSAGSAAGSGEFGSAEAAAKEAAAKAGSAGSAAGSGEFGSNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVV

SEQ ID NO: 107 - protein, 820 aa, synthetic construct
MIVKPMVRNNICLNAHPQGCKKGVEDQIEYTKKRITAEVKAGAKAPKNVLVLGCSNGYGLASRITAAFGYGAATIGVSFEKAGSETKYGTPGWYNNLAFDEAAKREGLYSVTIDGDAFSDEIKAQVIEEAKKKGIKFDLIVYSLASPVRTDPDTGIMHKSVLKPFGKTFTGKTVDPFTGELKEISAEPANDEEAAATVKVMGGEDWERWIKQLSKEGLLEEGCITLAYSYIGPEATQALYRKGTIGKAKEHLEATAHRLNKENPSIRAFVSVNKGLVTRASAVIPVIPLYLASLFKVMKEKGNHEGCIEQITRLYAERLYRKDGTIPVDEENRIRIDDWELEEDVQKAVSALMEKVTGENAESLTDLAGYRHDFLASNGFDVEGINYEAEVERFDRIKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASSYYHHHHHHLESTSLYKKAGSGSNLLATLRSTAAVLENENHVLEKEKEKLRKEKEQLLNKLEAYKGSAGSAAGSGEFGSAEAAAKEAAAKAGSAGSAAGSGEFGSSYYHHHHHHLESTSLYKKAGSGSKRIAYLRKKIAALKKDNANLEKDIANLENEIERLIKEIKTLENEVASHEQ

SEQ ID NO: 108 - protein, 826 aa, synthetic construct
MTREVVVVSGVRTAIGTFGGSLKDVAPAELGALVVREALARAQVSGDDVGHVVFGNVIQTEPRDMYLGRVAAVNGGVTINAPALTVNRLCGSGLQAIVSAAQTILLGDTDVAIGGGAESMSRAPYLAPAARWGARMGDAGLVDMMLGALHDPFHRIHMGVTAENVAKEYDISRAQQDEAALESHRRASAAIKAGYFKDQIVPVVSKGRKGDVTFDTDEHVRHDATIDDMTKLRPVFVKENGTVTAGNASGLNDAAAAVVMMERAEAERRGLKPLARLVSYGHAGVDPKAMGIGPVPATKIALERAGLQVSDLDVIEANEAFAAQACAVTKALGLDPAKVNPNGSGISLGHPIGATGALITVKALHELNRVQGRYALVTMCIGGGQGIAAIFERIKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASDVMWEYKWENTGDAELYGPFTSAQMQTWVSEGYFPDGVYCRKLDPPGGQFYNSKRIDFDLYTGSAGSAAGSGEFGSAEAAAKEAAAKAGSAGSAAGSGEFGSESDSVEFNNAISYVNKIKTRFLDHPEIYRSFLEILHTYQKEQLHTKGRPFRGMSEEEVFTEVANLFRGQEDLLSEFGQFLPEAKR

SEQ ID NO: 109 - protein, 849 aa, synthetic construct
MKLSTKLCWCGIKGRLRPQKQQQLHNTNLQMTELKKQKTAEQKTRPQNVGIKGIQIYIPTQCVNQSELEKFDGVSQGKYTIGLGQTNMSFVNDREDIYSMSLTVLSKLIKSYNIDTNKIGRLEVGTETLIDKSKSVKSVLMQLFGENTDVEGIDTLNACYGGTNALFNSLNWIESNAWDGRDAIVVCGDIAIYDKGAARPTGGAGTVAMWIGPDAPIVFDSVRASYMEHAYDFYKPDFTSEYPYVDGHFSLTCYVKALDQVYKSYSKKAISKGLVSDPAGSDALNVLKYFDYNVFHVPTCKLVTKSYGRLLYNDFRANPQLFPEVDAELATRDYDESLTDKNIEKTFVNVAKPFHKERVAQSLIVPTNTGNMYTASVYAAFASLLNYVGSDDLQGKRVGLFSYGSGLAASLYSCKIVGDVQHIIKELDITNKLAKRITETPKDYEAAIELRENAHLKKNFKPQGSIEHLQSGVYYLTNIDDKFRRSYDVKKKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASLGPLPPGWEVRSTVSGRIYFVDHNNRTTQFTDPRLHGSAGSAAGSGEFGSAEAAAKEAAAKAGSAGSAAGSGEFGSGAMGPLPPGWEKRTDSNGRVYFVNHNTRITQWEDPRS

SEQ ID NO: 110 - protein, 826 aa, synthetic construct
MVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASSYYHHHHHHLESTSLYKKAGSEFFRRERNKMAAAKCRNRRRELTDTLQAETDQLEDEKSALQTEIANLLKEKEKLEFILAAHRPACKIPDDLGFPEEMSLEGSAGSAAGSGEFGSAEAAAKEAAAKAGSAGSAAGSGEFGSSYYHHHHHHLESTSLYKKAGSGSQKVESLKQKIEELKQRKAQLKNDIANLEKEIAYAET

SEQ ID NO: 111 - protein, 1046 aa, synthetic construct
MSLPFLTSAPGKVIIFGEHSAVYNKPAVAASVSALRTYLLISESSAPDTIELDFPDISFNHKWSINDFNAITEDQVNSQKLAKAQQATDGLSQELVSLLDPLLAQLSESFHYHAAFCFLYMFVCLCPHAKNIKFSLKSTLPIGAGLGSSASISVSLALAMAYLGGLIGSNDLEKLSENDKHIVNQWAFIGEKCIHGTPSGIDNAVATYGNALLFEKDSHNGTINTNNFKFLDDFPAIPMILTYTRIPRSTKDLVARVRVLVTEKFPEVMKPILDAMGECALQGLEIMTKLSKCKGTDDEAVETNNELYEQLLELIRINHGLLVSIGVSHPGLELIKNLSDDLRIGSTKLTGAGGGGCSLTLLRRDITQEQIDSFKKKLQDDFSYETFETDLGGTGCCLLSAKNLNKDLKIKSLVFQLFENKTTTKQQIDDLLLPGNTNLPWTSKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASMEPAMEPETLEARINRATNPLNKELDWASINGFCEQLNEDFEGPPLATRLLAHKIQSPQEWEAIQALTVLETCMKSCGKRFHDEVGKFRFLNELIKVVSPKYLGSRTSEKVKNKILELLYSWTVGLPEEVKIAEAYQMLKKQGIVKSGSAGSAAGSGEFGSAEAAAKEAAAKAGSAGSAAGSGEFGSGAMGSMAEAEGESLESWLNKATNPSNRQEDWEYIIGFCDQINKELEGPQIAVRLLAHKIQSPQEWEALQALTVLEACMKNCGRRFHNEVGKFRFLNELIKVVSPKYLGDRVSEKVKTKVIELLYSWTMALPEEAKIKDAYHMLKRQGIVQSDPPIPVDRTLIPSPPPRPKN

SEQ ID NO: 112 - protein, 868 aa, synthetic construct
MSELRAFSAPGKALLAGGYLVLDTKYEAFVVGLSARMHAVAHPYGSLQGSDKFEVRVKSKQFKDGEWLYHISPKSGFIPVSIGGSKNPFIEKVIANVFSYFKPNMDDYCNRNLFVIDIFSDDAYHSQEDSVTEHRGNRRLSFHSHRIEEVPKTGLGSSAGGLVTVLTTALASFFVSDLENNVDKYREVIHNLAQVAHCQAQGKIGSGFDVAAAAYGSIRYRRFPPALISNLPDIGSATYGSKLAHLVDEEDWNITIKSNHLPSGLTLWMGDIKNGSETVKLVQKVKNWYDSHMPESLKIYTELDHANSRFMDGLSKLDRLHETHDDYSDQIFESLERNDCTCQKYPEITEVRDAVATIRRSFRKITKESGADIEPPVQTSLLDDCQTLKGVLTCLIPGAGGYDAIAVITKQDVDLRAQTANDKRFSKVQWLDVTQADWGVRKEKDPETYLDKKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASSYYHHHHHHLESTSLYKKAGSGSQKVEELKNKIAELENRNAVKKNRVAHLKQEIAYLKDELAAHEFEGSAGSAAGSGEFGSAEAAAKEAAAKAGSAGSAAGSGEFGSSYYHHHHHHLESTSLYKKAGSGSFENVTHEFILATLENENAKLRRLEAKLERELARLRNEVAWL

SEQ ID NO: 113 - protein, 1008 aa, synthetic construct
MTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKEKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASAMADLEQKVLEMEASTYDGVFIWKISDFPRKRQEAVAGRIPAIFSPAFYTSRYGYKMCLRIYLNGDGTGRGTHLSLFFVVMKGPNDALLRWPFNQKVTLMLLDQNNREHVIDAFRPDVTSSSFQRPVNDMNIASGCPLFCPVSKMEAKNSYVRDDAIFIKAIVDLTGLGSAGSAAGSGEFGSAEAAAKEAAAKAGSAGSAAGSGEFGSASIKLQSSDGEIFEVDVEIAKQSVTIKTMLEDLGMDDEGDDDPVPLPNVNAAILKKVIQWCTHHKDDPPPPEDDENKEKRTDDIPVWDQEFLKVDQGTLFELILAANYLDIKGLLDVTCKTVANMIKGKTPEEIRKTFNIKNDFTEEEEAQVRKENQWC

SEQ ID NO: 114 - protein, 717 aa, synthetic construct
MTADNNSMPHGAVSSYAKLVQNQTPEDILEEFPEIIPLQQRPNTRSSETSNDESGETCFSGHDEEQIKLMNENCIVLDWDDNAIGAGTKKVCHLMENIEKGLLHRAFSVFIFNEQGELLLQQRATEKITFPDLWTNTCCSHPLCIDDELGLKGKLDDKIKGAITAAVRKLDHELGIPEDETKTRGKFHFLNRIHYMAPSNEPWGEHEIDYILFYKINAKENLTVNPNVNEVRDFKWVSPNDLKTMFADPSYKFTPWFKIICENYLFNWWEQLDDLSEVENDRQIHRMLKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASSYYHHHHHHLESTSLYKKAGSGSNTVKELKNYIQELEERNAELKNLKEHLKFAKAELEFELAAHKFEGSAGSAAGSGEFGSAEAAAKEAAAKAGSAGSAAGSGEFGSSYYHHHHHHLESTSLYKKAGSGSQKVAQLKNRVAYKLKENAKLENIVARLENDNANLEKDIANLEKDIANLERDVAR

SEQ ID NO: 115 - protein, 798 aa, synthetic construct
MEAKIDELINNDPVWSSQNESLISKPYNHILLKPGKNFRLNLIVQINRVMNLPKDQLAIVSQIVELLHNSSLLIDDIEDNAPLRRGQTTSHLIWGVPSTINTANYMYFRAMQLVSQLTTKEPLYHWLITIFNEELINLHRGQGLDIYWRDFLPEIIPTQEMYLNMVMNKTGGLFRLTLRLMEALSPSSHHGHSLVPFINLLGIIYQIRDDYLNLKDFQMSSEKGFAEDITEGKLSFPIVHALNFTKTKGQTEQHNEILRILLLRTSDKDIKLKLIQILEFDTNSLAYTKNFINQLVNMIKNDNENKYLPDLASHSDTATNLHDELLYIIDHLSELKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASLCTMKKGPSGYGFNLHSDKSKPGQFIRSVDPDSPAEASGLRAQDRIVEVNGVCMEGKQHGDVVSAIRAGGDETKLLVVDREGSAGSAAGSGEFGSAEAAAKEAAAKAGSAGSAAGSGEFGSSSGAIIYTVELKRYGGPLGITISGTEEPFDPIIISSLTKGGLAERTGAIHIGDRILAINSSSLKGKPLSEAIHLLQMAGETVTLKIKKQTDAQPASS

SEQ ID NO: 116 - protein, 883 aa, synthetic construct
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKYKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASGNNLETYEWYNKSISRDKAEKLLLDTGKEGAFMVRDSRTPGTYTVSVFTKAIISENPCIKHYHIKETNDSPKRYYVAEKYVFDSIPLLIQYHQYNGGGLVTRLRYPVCGGSAGSAAGSGEFGSAEAAAKEAAAKAGSAGSAAGSGEFGSGSHPWFFGKIPRAKAEEMLSKQRHDGAFLIRESESAPGDFSLSVKFGNDVQHFKVLRDGAGKYFLWVVKFNSLNELVDYHRSTSVSRNQQIFLRDIEQVPQQPT

SEQ ID NO: 117 - protein, 665 aa, synthetic construct
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRKKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASGQDRSEATLIKRFKGEGVRYKAKLIGIDEVSAARGDKLCQDSMMKLKGVVAGARSKGEHKQKIFLTISFGGIKIFDEKTGALQHHHAVHEISYIAKDITDHRAFGYVCGKEGNHRFVAIKTAQAAEPVILDLRDLFQLIYELKQREELEKKAGSAGSAAGSGEFGSAEAAAKEAAAKAGSAGSAAGSGEFGSGSHMGSQFWVTSQKTEASERCGLQGSYILRVEAEKLTLLTLGAQSQILEPLLFWPYTLLRRYGRDKVMFSFEAGRRCPSGPGTFTFQTSQGNDIFQAVEAAIQQQKAQGKVGQAQDILRLEHHHHHH

SEQ ID NO: 118 - protein, 797 aa, synthetic construct
MGLSSVCTFSFQTNYHTLLNPHNNNPKTSLLCYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFIKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASAEYVRALFDFNGNDEEDLPFKKGDILRIRDKPEEQWWNAEDSEGKRGMIPVPYVEKYGSAGSAAGSGEFGSAEAAAKEAAAKAGSAGSAAGSGEFGSLIKHMRAEALFDFTGNSKLELNFKAGDVIFLLSRINKDWLEGTVRGATGIFPLSFVKILK

SEQ ID NO: 119 - protein, 3620 aa, synthetic construct
MGSAGSAAGSGEFGSAGSAAGSGEFGSAGSAAGSGEFSYYHHHHHHLESTSLYKKAGSGSARNAYLRKKIARLKKDNLQLERDEQNLEKIIANLRDEIARLENEVASHEQGSAGSAAGSGEFAEAAAKEAAAKAGSAGSAAGSGEFSYYHHHHHHLESTSLYKKAGSGSNLVAQLENEVASLENENETLKKKNLHKKDLIAYLEKEIANLRKKIEEGSAGSAAGSGEFGSAEAAAKEAAAKEAAAKEAAAKAGSAGSAAGSGEFGSSYYHHHHHHLESTSLYKKAGSGSQKVAELKNRVAVKLNRNEQLKNKVEELKNRNAYLKNELATLENEVARLENDVAEGSAGSAAGSGEFAEAAAKEAAAKAGSAGSAAGSGEFSYYHHHHHHLESTSLYKKAGSGSNEVTTLENDAAFIENENAYLEKEIARLRKEKAALRNRLAHKKGSAGSAAGSGEFGSAEAAAKEAAAKEAAAKEAAAKAGSAGSAAGSGEFGSRPPTISNPPPLISSAKHPSVGSAGSAAGSGEFAEAAAKEAAAKAGSAGSAAGSGEFNFLQSRPEPTAPPEESFRSGGSAGSAAGSGEFGSAEAAAKEAAAKEAAAKEAAAKAGSAGSAAGSGEFGSSKGTGLNPNAKVWQEIAPGNGSAGSAAGSGEFAEAAAKEAAAKAGSAGSAAGSGEFPDGGTTFEHLWSSLEPDSTYGSAGSAAGSGEFGSAEAAAKEAAAKEAAAKEAAAKAGSAGSAAGSGEFGSSYYHHHHHHLESTSLYKKAGSGSKRIAYLRKKIAALKKDNANLEKDIANLENEIERLIKEIKTLENEVASHEQGSAGSAAGSGEFAEAAAKEAAAKAGSAGSAAGSGEFSYYHHHHHHLESTSLYKKAGSGSNLLATLRSTAAVLENENHVLEKEKEKLRKEKEQLLNKLEAYKGSAGSAAGSGEFGSAEAAAKEAAAKEAAAKEAAAKAGSAGSAAGSGEFGSPATSQHPPPPPGHRSQAPSHGSAGSAAGSGEFAEAAAKEAAAKAGSAGSAAGSGEFELNSLLILLEAAEYLERRDRGSAGSAAGSGEFGSAEAAAKEAAAKEAAAKEAAAKAGSAGSAAGSGEFGSRPPTISNPPPLISSAKHPSVGSAGSAAGSGEFAEAAAKEAAAKAGSAGSAAGSGEFNFLQSRPEPTAPPEESFRSGGSAGSAAGSGEFGSAEAAAKEAAAKEAAAKEAAAKAGSAGSAAGSGEFGSSKGTGLNPNAKVWQEIAPGNGSAGSAAGSGEFAEAAAKEAAAKAGSAGSAAGSGEFPDGGTTFEHLWSSLEPDSTYGSAGSAAGSGEFGSAEAAAKEAAAKEAAAKEAAAKAGSAGSAAGSGEFGSSYYHHHHHHLESTSLYKKAGSGSKRIAYLRKKIAALKKDNANLEKDIANLENEIERLIKEIKTLENEVASHEQGSAGSAAGSGEFAEAAAKEAAAKAGSAGSAAGSGEFSYYHHHHHHLESTSLYKKAGSGSNLLATLRSTAAVLENENHVLEKEKEKLRKEKEQLLNKLEAYKGSAGSAAGSGEFGSAEAAAKEAAAKEAAAKEAAAKAGSAGSAAGSGEFGSALVDDAADYEPPPSNNEEALGSAGSAAGSGEFAEAAAKEAAAKAGSAGSAAGSGEFRELFDDPSYVNVQNLDKARQGSAGSAAGSGEFGSAEAAAKEAAAKEAAAKEAAAKAGSAGSAAGSGEFGSKNTKSMNFDNPVYRKTTEEEGSAGSAAGSGEFAEAAAKEAAAKAGSAGSAAGSGEFRSLPSTWIENKLYGMSDPNWGSAGSAAGSGEFGSAEAAAKEAAAKEAAAKEAAAKAGSAGSAAGSGEFGSVVDNSPPPALPPKKRQSAPSGSAGSAAGSGEFAEAAAKEAAAKAGSAGSAAGSGEFTQRSKPQPAVPPRPSADLILGSAGSAAGSGEFGSAEAAAKEAAAKEAAAKEAAAKAGSAGSAAGSGEFGSTDEEREETEEEVYLLNSTTLGSAGSAAGSGEFAEAAAKEAAAKAGSAGSAAGSGEFDGNVSGTQRLDSATVRTYSCGSAGSAAGSGEFGSAEAAAKEAAAKEAAAKEAAAKAGSAGSAAGSGEFGSSYYHHHHHHLESTSLYKKAGSGSQKVAQLKNRVAYKLKENAKLENIVARLENDNANLEKDIANLEKDIANLERDVARGSAGSAAGSGEFAEAAAKEAAAKAGSAGSAAGSGEFSYYHHHHHHLESTSLYKKAGSGSNTVKELKNYIQELEERNAELKNLKEHLKFAKAELEFELAAHKFEGSAGSAAGSGEFGSAEAAAKEAAAKEAAAKEAAAKAGSAGSAAGSGEFGSHDDSLPHPQQATDDSGHESDGSAGSAAGSGEFAEAAAKEAAAKAGSAGSAAGSGEFGSPNAGSVEQTPKKPGLRRRGSAGSAAGSGEFGSAEAAAKEAAAKEAAAKEAAAKAGSAGSAAGSGEFGSSYYHHHHHHLESTSLYKKAGSGSFENVTHEFILATLENENAKLRRLEAKLERELARLRNEVAWLGSAGSAAGSGEFAEAAAKEAAAKAGSAGSAAGSGEFSYYHHHHHHLESTSLYKKAGSGSQKVEELKNKIAELENRNAVKKNRVAHLKQEIAYLKDELAAHEFEGSAGSAAGSGEFGSAEAAAKEAAAKEAAAKEAAAKAGSAGSAAGSGEFGSVSSTKLVSFHDDSDEDLLHIGSAGSAAGSGEFAEAAAKEAAAKAGSAGSAAGSGEFAAATPISTFHDDSDEDLLHVGSAGSAAGSGEFGSAEAAAKEAAAKEAAAKEAAAKAGSAGSAAGSGEFGSSYYHHHHHHLESTSLYKKAGSGSQKVESLKQKIEELKQRKAQLKNDIANLEKEIAYAETGSAGSAAGSGEFAEAAAKEAAAKAGSAGSAAGSGEFSYYHHHHHHLESTSLYKKAGSEFFRRERNKMAAAKCRNRRRELTDTLQAETDQLEDEKSALQTEIANLLKEKEKLEFILAAHRPACKIPDDLGFPEEMSLEGSAGSAAGSGEFGSAEAAAKEAAAKEAAAKEAAAKAGSAGSAAGSGEFGSFQMPADTPPPAYLPPEDPMTGSAGSAAGSGEFAEAAAKEAAAKAGSAGSAAGSGEFERESNEEPPPPYEDPYWGNGGSAGSAAGSGEFGSAEAAAKEAAAKEAAAKEAAAKAGSAGSAAGSGEFGSSYYHHHHHHLESTSLYKKAGSGSQKVAELKNRVAVKLNRNEQLKNKVEELKNRNAYLKNELATLENEVARLENDVAEGSAGSAAGSGEFAEAAAKEAAAKAGSAGSAAGSGEFSYYHHHHHHLESTSLYKKAGSGSNEVTTLENDAAFIENENAYLEKEIARLRKEKAALRNRLAHKKSYYHHHHHHLESTSLYKKAGSGSARNAYLRKKIARLKKDNLQLERDEQNLEKIIANLRDEIARLENEVASHEQGSAGSAAGSGEFAEAAAKEAAAKAGSAGSAAGSGEFSYYHHHHHHLESTSLYKKAGSGSNLVAQLENEVASLENENETLKKKNLHKKDLIAYLEKEIANLRKKIEEGSAGSAAGSGEFGSAEAAAKEAAAKEAAAKEAAAKAGSAGSAAGSGEFGSEQKLISEEDLEQKLISEEDLEQKLISEEDLGSAGSAAGSGEFGSAGSAAGSGEFGSAGSAAGSGEF

SEQ ID NO: 120 - protein, 452 aa, synthetic construct
MGSAGSAAGSGEFGSAGSAAGSGEFGSAGSAAGSGEFSYYHHHHHHLESTSLYKKAGSGSARNAYLRKKIARLKKDNLQLERDEQNLEKIIANLRDEIARLENEVASHEQGSAGSAAGSGEFAEAAAKEAAAKAGSAGSAAGSGEFSYYHHHHHHLESTSLYKKAGSGSNLVAQLENEVASLENENETLKKKNLHKKDLIAYLEKEIANLRKKIEEGSAGSAAGSGEFGSAEAAAKEAAAKEAAAKEAAAKAGSAGSAAGSGEFGSSATRELDELMASLSDFKIQGGSAGSAAGSGEFAEAAAKEAAAKAGSAGSAAGSGEFDLALSENWAQEFLAAGDAVDGSAGSAAGSGEFGSAEAAAKEAAAKEAAAKEAAAKAGSAGSAAGSGEFGSDYKDDDDKDYKDDDDKDYKDDDDKGSAGSAAGSGEFGSAGSAAGSGEFGSAGSAAGSGEF

SEQ ID NO: 121 - DNA, 3303 bp, synthetic construct
  source 1..3303: mol_type = other DNA
atgtctgctaaagctatttctgaacaaactggtaaagaattgttgtataaatttatttgtactacttctgctattcaaaatagatttaaatatgctagagttactccagatactgattgggctagattgttgcaagatcatccatggttgttgtctcaaaatttggttgttaaaccagatcaattgattaaaagaagaggtaaattgggtttggttggtgttaatttgactttggatggtgttaaatcttggttgaaaccaagattgggtcaagaagctactgttggtaaagctactggttttttgaaaaattttttgattgaaccatttgttccacattctcaagctgaagaattttatgtttgtatttatgctactagagaaggtgattatgttttgtttcatcatgaaggtggtgttgatgttggtgatgttgatgctaaagctcaaaaattgttggttggtgttgatgaaaaattgaatccagaagatattaaaaaacatttgttggttcatgctccagaagataaaaaagaaattttggcttcttttatttctggtttgtttaatttttatgaagatttgtattttacttatttggaaattaatccattggttgttactaaagatggtgtttatgttttggatttggctgctaaagttgatgctactgctgattatatttgtaaagttaaatggggtgatattgaatttccaccaccatttggtagagaagcttatccagaagaagcttatattgctgatttggatgctaaatctggtgcttctttgaaattgactttgttgaatccaaaaggtagaatttggactatggttgctggtggtggtgcttctgttgtttattctgatactatttgtgatttgggtggtgttaatgaattggctaattatggtgaatattctggtgctccatctgaacaacaaacttatgattatgctaaaactattttgtctttgatgactagagaaaaacatccagatggtaaaattttgattattggtggttctattgctaattttactaatgttgctgctacttttaaaggtattgttagagctattagagattatcaaggtccattgaaagaacatgaagttactatttttgttagaagaggtggtccaaattatcaagaaggtttgagagttatgggtgaagttggtaaaactactggtattccaattcatgtttttggtactgaaactcatatgactgctattgttggtatggctttgggtcatagaccaattccaaatcaaccaccaactgctgctcatactgctaattttttgttgaatgcttctggttctacttctactccagctccatctagaactgcttctttttctgaatctagagctgatgaagttgctccagctaaaaaagctaaaccagctatgccacaagattctgttccatctccaagatctttgcaaggtaaatctactactttgttttctagacatactaaagctattgtttggggtatgcaaactagagctgttcaaggtatgttggattttgattatgtttgttctagagatgaaccatctgttgctgctatggtttatccatttactggtgatcataaacaaaaattttattggggtcataaagaaattttgattccagtttttaaaaatatggctgatgctatgagaaaacatccagaagttgatgttttgattaattttgcttctttgagatctgcttatgattctactatggaaactatgaattatgctcaaattagaactattgctattattgctgaaggtattccagaagctttgactagaaaattgattaaaaaagctgatcaaaaaggtgttactattattggtccagctactgttggtggtattaaaccaggttgttttaaaattggtaatactggtggtatgttggataatattttggcttctaaattgtatagaccaggttctgttgcttatgtttctagatctggtggtatgtctaatgaattgaataatattatttctagaactactgatggtgtttatgaaggtgttgctattggtggtgatagatatccaggttctacttttatggatcatgttttgagatatcaagatactccaggtgttaaaatgattgttgttttgggtgaaattggtggtactgaagaatataaaatttgtagaggtattaaagaaggtagattgactaaaccaattgtttgttggtgtattggtacttgtgctactatgttttcttctgaagttcaatttggtcatgctggtgcttgtgctaatcaagcttctgaaactgctgttgctaaaaatcaagctttgaaagaagctggtgtttttgttccaagatcttttgatgaattgggtgaaattattcaatctgtttatgaagatttggttgctaatggtgttattgttccagctcaagaagttccaccaccaactgttccaatggattattcttgggctagagaattgggtttgattagaaaaccagcttcttttatgacttctatttgtgatgaaagaggtcaagaattgatttatgctggtatgccaattactgaagtttttaaagaagaaatgggtattggtggtgttttgggtttgttgtggtttcaaaaaagattgccaaaatattcttgtcaatttattgaaatgtgtttgatggttactgctgatcatggtccagctgtttctggtgctcataatactattatttgtgctagagctggtaaagatttggtttcttctttgacttctggtttgttgactattggtgatagatttggtggtgctttggatgctgctgctaaaatgttttctaaagcttttgattctggtattattccaatggaatttgttaataaaatgaaaaaagaaggtaaattgattatgggtattggtcatagagttaaatctattaataatccagatatgagagttcaaattttgaaagattatgttagacaacattttccagctactccattgttggattatgctttggaagttgaaaaaattactacttctaaaaaaccaaatttgattttgaatgttgatggtttgattggtgttgcttttgttgatatgttgagaaattgtggttcttttactagagaagaagctgatgaatatattgatattggtgctttgaatggtatttttgttttgggtagatctatgggttttattggtcattatttggatcaaaaaagattgaaacaaggtttgtatagacatccatgggatgatatttcttatgttttgccagaacatatgtctatg

SEQ ID NO: 122 - DNA, 1182 bp, synthetic construct
  source 1..1182: mol_type = other DNA
atgaaaaattgtgttattgtttctgctgttagaactgctattggttcttttaatggttctttggcttctacttctgctattgatttgggtgctactgttattaaagctgctattgaaagagctaaaattgattctcaacatgttgatgaagttattatgggtaatgttttgcaagctggtttgggtcaaaatccagctagacaagctttgttgaaatctggtttggctgaaactgtttgtggttttactgttaataaagtttgtggttctggtttgaaatctgttgctttggctgctcaagctattcaagctggtcaagctcaatctattgttgctggtggtatggaaaatatgtctttggctccatatttgttggatgctaaagctagatctggttatagattgggtgatggtcaagtttatgatgttattttgagagatggtttgatgtgtgctactcatggttatcatatgggtattactgctgaaaatgttgctaaagaatatggtattactagagaaatgcaagatgaattggctttgcattctcaaagaaaagctgctgctgctattgaatctggtgcttttactgctgaaattgttccagttaatgttgttactagaaaaaaaacttttgttttttctcaagatgaatttccaaaagctaattctactgctgaagctttgggtgctttgagaccagcttttgataaagctggtactgttactgctggtaatgcttctggtattaatgatggtgctgctgctttggttattatggaagaatctgctgctttggctgctggtttgactccattggctagaattaaatcttatgcttctggtggtgttccaccagctttgatgggtatgggtccagttccagctactcaaaaagctttgcaattggctggtttgcaattggctgatattgatttgattgaagctaatgaagcttttgctgctcaatttttggctgttggtaaaaatttgggttttgattctgaaaaagttaatgttaatggtggtgctattgctttgggtcatccaattggtgcttctggtgctagaattttggttactttgttgcatgctatgcaagctagagataaaactttgggtttggctactttgtgtattggtggtggtcaaggtattgctatggttattgaaagattgaat

SEQ ID NO: 123 - DNA, 846 bp, synthetic construct
  source 1..846: mol_type = other DNA
atgaaaaaagtttgtgttattggtgctggtactatgggttctggtattgctcaagcttttgctgctaaaggttttgaagttgttttgagagatattaaagatgaatttgttgatagaggtttggattttattaataaaaatttgtctaaattggttaaaaaaggtaaaattgaagaagctactaaagttgaaattttgactagaatttctggtactgttgatttgaatatggctgctgattgtgatttggttattgaagctgctgttgaaagaatggatattaaaaaacaaatttttgctgatttggataatatttgtaaaccagaaactattttggcttctaatacttcttctttgtctattactgaagttgcttctgctactaaaagaccagataaagttattggtatgcatttttttaatccagctccagttatgaaattggttgaagttattagaggtattgctacttctcaagaaacttttgatgctgttaaagaaacttctattgctattggtaaagatccagttgaagttgctgaagctccaggttttgttgttaatagaattttgattccaatgattaatgaagctgttggtattttggctgaaggtattgcttctgttgaagatattgataaagctatgaaattgggtgctaatcatccaatgggtccattggaattgggtgattttattggtttggatatttgtttggctattatggatgttttgtattctgaaactggtgattctaaatatagaccacatactttgttgaaaaaatatgttagagctggttggttgggtagaaaatctggtaaaggtttttatgattattctaaa

SEQ ID NO: 124 - DNA, 783 bp, synthetic construct
  source 1..783: mol_type = other DNA
atggaattgaataatgttattttggaaaaagaaggtaaagttgctgttgttactattaatagaccaaaagctttgaatgctttgaattctgatactttgaaagaaatggattatgttattggtgaaattgaaaatgattctgaagttttggctgttattttgactggtgctggtgaaaaatcttttgttgctggtgctgatatttctgaaatgaaagaaatgaatactattgaaggtagaaaatttggtattttgggtaataaagtttttagaagattggaattgttggaaaaaccagttattgctgctgttaatggttttgctttgggtggtggttgtgaaattgctatgtcttgtgatattagaattgcttcttctaatgctagatttggtcaaccagaagttggtttgggtattactccaggttttggtggtactcaaagattgtctagattggttggtatgggtatggctaaacaattgatttttactgctcaaaatattaaagctgatgaagctttgagaattggtttggttaataaagttgttgaaccatctgaattgatgaatactgctaaagaaattgctaataaaattgtttctaatgctccagttgctgttaaattgtctaaacaagctattaatagaggtatgcaatgtgatattgatactgctttggcttttgaatctgaagcttttggtgaatgtttttctactgaagatcaaaaagatgctatgactgcttttattgaaaaaagaaaaattgaaggttttaaaaataga

SEQ ID NO: 125 - DNA, 1191 bp, synthetic construct
  source 1..1191: mol_type = other DNA
atgattgttaaaccaatggttagaaataatatttgtttgaatgctcatccacaaggttgtaaaaaaggtgttgaagatcaaattgaatatactaaaaaaagaattactgctgaagttaaagctggtgctaaagctccaaaaaatgttttggttttgggttgttctaatggttatggtttggcttctagaattactgctgcttttggttatggtgctgctactattggtgtttcttttgaaaaagctggttctgaaactaaatatggtactccaggttggtataataatttggcttttgatgaagctgctaaaagagaaggtttgtattctgttactattgatggtgatgctttttctgatgaaattaaagctcaagttattgaagaagctaaaaaaaaaggtattaaatttgatttgattgtttattctttggcttctccagttagaactgatccagatactggtattatgcataaatctgttttgaaaccatttggtaaaacttttactggtaaaactgttgatccatttactggtgaattgaaagaaatttctgctgaaccagctaatgatgaagaagctgctgctactgttaaagttatgggtggtgaagattgggaaagatggattaaacaattgtctaaagaaggtttgttggaagaaggttgtattactttggcttattcttatattggtccagaagctactcaagctttgtatagaaaaggtactattggtaaagctaaagaacatttggaagctactgctcatagattgaataaagaaaatccatctattagagcttttgtttctgttaataaaggtttggttactagagcttctgctgttattccagttattccattgtatttggcttctttgtttaaagttatgaaagaaaaaggtaatcatgaaggttgtattgaacaaattactagattgtatgctgaaagattgtatagaaaagatggtactattccagttgatgaagaaaatagaattagaattgatgattgggaattggaagaagatgttcaaaaagctgtttctgctttgatggaaaaagttactggtgaaaatgctgaatctttgactgatttggctggttatagacatgattttttggcttctaatggttttgatgttgaaggtattaattatgaagctgaagttgaaagatttgatagaatt

SEQ ID NO: 126 - DNA, 1182 bp, synthetic construct
  source 1..1182: mol_type = other DNA
atgactagagaagttgttgttgtttctggtgttagaactgctattggtacttttggtggttctttgaaagatgttgctccagctgaattgggtgctttggttgttagagaagctttggctagagctcaagtttctggtgatgatgttggtcatgttgtttttggtaatgttattcaaactgaaccaagagatatgtatttgggtagagttgctgctgttaatggtggtgttactattaatgctccagctttgactgttaatagattgtgtggttctggtttgcaagctattgtttctgctgctcaaactattttgttgggtgatactgatgttgctattggtggtggtgctgaatctatgtctagagctccatatttggctccagctgctagatggggtgctagaatgggtgatgctggtttggttgatatgatgttgggtgctttgcatgatccatttcatagaattcatatgggtgttactgctgaaaatgttgctaaagaatatgatatttctagagctcaacaagatgaagctgctttggaatctcatagaagagcttctgctgctattaaagctggttattttaaagatcaaattgttccagttgtttctaaaggtagaaaaggtgatgttacttttgatactgatgaacatgttagacatgatgctactattgatgatatgactaaattgagaccagtttttgttaaagaaaatggtactgttactgctggtaatgcttctggtttgaatgatgctgctgctgctgttgttatgatggaaagagctgaagctgaaagaagaggtttgaaaccattggctagattggtttcttatggtcatgctggtgttgatccaaaagctatgggtattggtccagttccagctactaaaattgctttggaaagagctggtttgcaagtttctgatttggatgttattgaagctaatgaagcttttgctgctcaagcttgtgctgttactaaagctttgggtttggatccagctaaagttaatccaaatggttctggtatttctttgggtcatccaattggtgctactggtgctttgattactgttaaagctttgcatgaattgaatagagttcaaggtagatatgctttggttactatgtgtattggtggtggtcaaggtattgctgctatttttgaaagaatt

SEQ ID NO: 127 - DNA, 1476 bp, synthetic construct
  source 1..1476: mol_type = other DNA
atgaaactctcaactaaactttgttggtgtggtattaaaggaagacttaggccgcaaaagcaacaacaattacacaatacaaacttgcaaatgactgaactaaaaaaacaaaagaccgctgaacaaaaaaccagacctcaaaatgtcggtattaaaggtatccaaatttacatcccaactcaatgtgtcaaccaatctgagctagagaaatttgatggcgtttctcaaggtaaatacacaattggtctgggccaaaccaacatgtcttttgtcaatgacagagaagatatctactcgatgtccctaactgttttgtctaagttgatcaagagttacaacatcgacaccaacaaaattggtagattagaagtcggtactgaaactctgattgacaagtccaagtctgtcaagtctgtcttgatgcaattgtttggtgaaaacactgacgtcgaaggtattgacacgcttaatgcctgttacggtggtaccaacgcgttgttcaactctttgaactggattgaatctaacgcatgggatggtagagacgccattgtagtttgcggtgatattgccatctacgataagggtgccgcaagaccaaccggtggtgccggtactgttgctatgtggatcggtcctgatgctccaattgtatttgactctgtaagagcttcttacatggaacacgcctacgatttttacaagccagatttcaccagcgaatatccttacgtcgatggtcatttttcattaacttgttacgtcaaggctcttgatcaagtttacaagagttattccaagaaggctatttctaaagggttggttagcgatcccgctggttcggatgctttgaacgttttgaaatatttcgactacaacgttttccatgttccaacctgtaaattggtcacaaaatcatacggtagattactatataacgatttcagagccaatcctcaattgttcccagaagttgacgccgaattagctactcgcgattatgacgaatctttaaccgataagaacattgaaaaaacttttgttaatgttgctaagccattccacaaagagagagttgcccaatctttgattgttccaacaaacacaggtaacatgtacaccgcatctgtttatgccgcctttgcatctctattaaactatgttggatctgacgacttacaaggcaagcgtgttggtttattttcttacggttccggtttagctgcatctctatattcttgcaaaattgttggtgacgtccaacatattatcaaggaattagatattactaacaaattagccaagagaatcaccgaaactccaaaggattacgaagctgccatcgaattgagagaaaatgcccatttgaagaagaacttcaaacctcaaggttccattgagcatttgcaaagtggtgtttactacttgaccaacatcgatgacaaatttagaagatcttacgatgttaaaaaataa

SEQ ID NO: 128 - DNA, 1143 bp, synthetic construct
  source 1..1143: mol_type = other DNA
atggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacagg

SEQ ID NO: 129 - DNA, 1332 bp, synthetic construct
  source 1..1332: mol_type = other DNA
atgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataa

SEQ ID NO: 130 - DNA, 1356 bp, synthetic construct
  source 1..1356: mol_type = other DNA
atgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataa

SEQ ID NO: 131 - DNA, 1191 bp, synthetic construct
  source 1..1191: mol_type = other DNA
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

SEQ ID NO: 132 - DNA, 867 bp, synthetic construct
  source 1..867: mol_type = other DNA
atgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataa

SEQ ID NO: 133 - DNA, 1059 bp, synthetic construct
  source 1..1059: mol_type = other DNA
atggcttcagaaaaggaaataagaagagaaagattcttgaacgtattcccaaagttagttgaagaattgaacgctagtttgttagcttatggtatgcctaaagaagcctgcgattggtatgctcactctttaaactacaatactccaggtggtaaattgaatagaggtttgagtgtagttgatacttatgctatcttgtctaacaaaaccgttgaacaattaggtcaagaagaatacgaaaaggtcgctatcttgggttggtgtattgaattgttgcaagcatactttttggttgccgatgacatgatggataagtctataacaagaagaggtcaaccatgctggtacaaagttccagaagttggtgaaatagccataaatgatgcttttatgttggaagccgctatctataaattgttgaagtcacatttcagaaacgaaaagtactacatcgatattaccgaattattccacgaagttactttccaaacagaattgggtcaattgatggatttgataactgcacctgaagataaagttgacttgtcaaagttttccttgaagaaacattcattcatcgtcacctttgaaactgcttattactccttctatttgccagtcgccttggctatgtacgtagctggtattactgatgaaaaagacttgaagcaagcaagagatgttttgatacctttgggtgaatacttccaaatccaagatgactacttagactgtttcggtactccagaacaaataggtaaaatcggtacagatattcaagacaataagtgcagttgggttattaacaaggctttggaattagcatctgccgaacaaagaaagactttggatgaaaactacggtaaaaaggactcagttgctgaagcaaagtgtaagaaaatttttaatgatttgaagattgaacaattgtaccatgaatacgaagaatccatcgctaaagacttaaaggcaaagattagtcaagttgatgaatcaagaggttttaaagccgacgttttgacagctttcttgaataaggtctacaagagatcaaagtag

SEQ ID NO: 134 - DNA, 1155 bp, synthetic construct
  source 1..1155: mol_type = other DNA
atgaatcatttgagagctgaaggtccagcttctgttttggctattggtactgctaatccagaaaatattttgttgcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggttgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggcttgtttgtttagaggtccatctgaatctgatttggaattgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaatctgataaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagagttgttgttagatctgttccaattaaatat

SEQ ID NO: 135 - DNA, 303 bp, synthetic construct
  source 1..303: mol_type = other DNA
atggctgttaaacatttgattgttttgaaatttaaagatgaaattactgaagctcaaaaagaagaattttttaaaacttatgttaatttggttaatattattccagctatgaaagatgtttattggggtaaagatgttactcaaaaaaataaagaagaaggttatactcatattgttgaagttacttttgaatctgttgaaactattcaagattatattattcatccagctcatgttggttttggtgatgtttatagatctttttgggaaaaattgttgatttttgattatactccaagaaaa

SEQ ID NO: 136 - DNA, 1185 bp, synthetic construct
  source 1..1185: mol_type = other DNA
atgggtttgtcttctgtttgtactttttcttttcaaactaattatcatactttgttgaatccacataataataatccaaaaacttctttgttgtgttatagacatccaaaaactccaattaaatattcttataataattttccatctaaacattgttctactaaatcttttcatttgcaaaataaatgttctgaatctttgtctattgctaaaaattctattagagctgctactactaatcaaactgaaccaccagaatctgataatcattctgttgctactaaaattttgaattttggtaaagcttgttggaaattgcaaagaccatatactattattgcttttacttcttgtgcttgtggtttgtttggtaaagaattgttgcataatactaatttgatttcttggtctttgatgtttaaagcttttttttttttggttgctattttgtgtattgcttcttttactactactattaatcaaatttatgatttgcatattgatagaattaataaaccagatttgccattggcttctggtgaaatttctgttaatactgcttggattatgtctattattgttgctttgtttggtttgattattactattaaaatgaaaggtggtccattgtatatttttggttattgttttggtatttttggtggtattgtttattctgttccaccatttagatggaaacaaaatccatctactgcttttttgttgaattttttggctcatattattactaattttactttttattatgcttctagagctgctttgggtttgccatttgaattgagaccatcttttacttttttgttggcttttatgaaatctatgggttctgctttggctttgattaaagatgcttctgatgttgaaggtgatactaaatttggtatttctactttggcttctaaatatggttctagaaatttgactttgttttgttctggtattgttttgttgtcttatgttgctgctattttggctggtattatttggccacaagcttttaattctaatgttatgttgttgtctcatgctattttggctttttggttgattttgcaaactagagattttgctttgactaattatgatccagaagctggtagaagattttatgaatttatgtggaaattgtattatgctgaatatttggtttatgtttttatt

SEQ ID NO: 137 - DNA, 6702 bp, synthetic construct
  source 1..6702: mol_type = other DNA
atgagcgaagaaagcttattcgagtcttctccacagaagatggagtacgaaattacaaactactcagaaagacatacagaacttccaggtcatttcattggcctcaatacagtagataaactagaggagtccccgttaagggactttgttaagagtcacggtggtcacacggtcatatccaagatcctgatagcaaataatggtattgccgccgtgaaagaaattagatccgtcagaaaatgggcatacgagacgttcggcgatgacagaaccgtccaattcgtcgccatggccaccccagaagatctggaggccaacgcagaatatatccgtatggccgatcaatacattgaagtgccaggtggtactaataataacaactacgctaacgtagacttgatcgtagacatcgccgaaagagcagacgtagacgccgtatgggctggctggggtcacgcctccgagaatccactattgcctgaaaaattgtcccagtctaagaggaaagtcatctttattgggcctccaggtaacgccatgaggtctttaggtgataaaatctcctctaccattgtcgctcaaagtgctaaagtcccatgtattccatggtctggtaccggtgttgacaccgttcacgtggacgagaaaaccggtctggtctctgtcgacgatgacatctatcaaaagggttgttgtacctctcctgaagatggtttacaaaaggccaagcgtattggttttcctgtcatgattaaggcatccgaaggtggtggtggtaaaggtatcagacaagttgaacgtgaagaagatttcatcgctttataccaccaggcagccaacgaaattccaggctcccccattttcatcatgaagttggccggtagagcgcgtcacttggaagttcaactgctagcagatcagtacggtacaaatatttccttgttcggtagagactgttccgttcagagacgtcatcaaaaaattatcgaagaagcaccagttacaattgccaaggctgaaacatttcacgagatggaaaaggctgccgtcagactggggaaactagtcggttatgtctctgccggtaccgtggagtatctatattctcatgatgatggaaaattctactttttagaattgaacccaagattacaagtcgagcatccaacaacggaaatggtctccggtgttaacttacctgcagctcaattacaaatcgctatgggtatccctatgcatagaataagtgacattagaactttatatggtatgaatcctcattctgcctcagaaatcgatttcgaattcaaaactcaagatgccaccaagaaacaaagaagacctattccaaagggtcattgtaccgcttgtcgtatcacatcagaagatccaaacgatggattcaagccatcgggtggtactttgcatgaactaaacttccgttcttcctctaatgtttggggttacttctccgtgggtaacaatggtaatattcactccttttcggactctcagttcggccatatttttgcttttggtgaaaatagacaagcttccaggaaacacatggttgttgccctgaaggaattgtccattaggggtgatttcagaactactgtggaatacttgatcaaacttttggaaactgaagatttcgaggataacactattaccaccggttggttggacgatttgattactcataaaatgaccgctgaaaagcctgatccaactcttgccgtcatttgcggtgccgctacaaaggctttcttagcatctgaagaagcccgccacaagtatatcgaatccttacaaaagggacaagttctatctaaagacctactgcaaactatgttccctgtagattttatccatgagggtaaaagatacaagttcaccgtagctaaatccggtaatgaccgttacacattatttatcaatggttctaaatgtgatatcatactgcgtcaactatctgatggtggtcttttgattgccataggcggtaaatcgcataccatctattggaaagaagaagttgctgctacaagattatccgttgactctatgactactttgttggaagttgaaaacgatccaacccagttgcgtactccatcccctggtaaattggttaaattcttggtggaaaatggtgaacacattatcaagggccaaccatatgcagaaattgaagttatgaaaatgcaaatgcctttggtttctcaagaaaatggtatcgtccagttattaaagcaacctggttctaccattgttgcaggtgatatcatggctattatgactcttgacgatccatccaaggtcaagcacgctctaccatttgaaggtatgctgccagattttggttctccagttatcgaaggaaccaaacctgcctataaattcaagtcattagtgtctactttggaaaacattttgaagggttatgacaaccaagttattatgaacgcttccttgcaacaattgatagaggttttgagaaatccaaaactgccttactcagaatggaaactacacatctctgctttacattcaagattgcctgctaagctagatgaacaaatggaagagttagttgcacgttctttgagacgtggtgctgttttcccagctagacaattaagtaaattgattgatatggccgtgaagaatcctgaatacaaccccgacaaattgctgggcgccgtcgtggaaccattggcggatattgctcataagtactctaacgggttagaagcccatgaacattctatatttgtccatttcttggaagaatattacgaagttgaaaagttattcaatggtccaaatgttcgtgaggaaaatatcattctgaaattgcgtgatgaaaaccctaaagatctagataaagttgcgctaactgttttgtctcattcgaaagtttcagcgaagaataacctgatcctagctatcttgaaacattatcaaccattgtgcaagttatcttctaaagtttctgccattttctctactcctctacaacatattgttgaactagaatctaaggctaccgctaaggtcgctctacaagcaagagaaattttgattcaaggcgctttaccttcggtcaaggaaagaactgaacaaattgaacatatcttaaaatcctctgttgtgaaggttgcctatggctcatccaatccaaagcgctctgaaccagatttgaatatcttgaaggacttgatcgattctaattacgttgtgttcgatgttttacttcaattcctaacccatcaagacccagttgtgactgctgcagctgctcaagtctatattcgtcgtgcttatcgtgcttacaccataggagatattagagttcacgaaggtgtcacagttccaattgttgaatggaaattccaactaccttcagctgcgttctccacctttccaactgttaaatctaaaatgggtatgaacagggctgtttctgtttcagatttgtcatatgttgcaaacagtcagtcatctccgttaagagaaggtattttgatggctgtggatcatttagatgatgttgatgaaattttgtcacaaagtttggaagttattcctcgtcaccaatcttcttctaacggacctgctcctgatcgttctggtagctccgcatcgttgagtaatgttgctaatgtttgtgttgcttctacagaaggtttcgaatctgaagaggaaattttggtaaggttgagagaaattttggatttgaataagcaggaattaatcaatgcttctatccgtcgtatcacatttatgttcggttttaaagatgggtcttatccaaagtattatacttttaacggtccaaattataacgaaaatgaaacaattcgtcacattgagccggctttggccttccaactggaattaggaagattgtccaacttcaacattaaaccaattttcactgataatagaaacatccatgtctacgaagctgttagtaagacttctccattggataagagattctttacaagaggtattattagaacgggtcatatccgtgatgacatttctattcaagaatatctgacttctgaagctaacagattgatgagtgatatattggataatttagaagtcaccgacacttcaaattctgatttgaatcatatcttcatcaacttcattgcggtgtttgatatctctccagaagatgtcgaagccgccttcggtggtttcttagaaagatttggtaagagattgttgagattgcgtgtttcttctgccgaaattagaatcatcatcaaagatcctcaaacaggtgccccagtaccattgcgtgccttgatcaataacgtttctggttatgttatcaaaacagaaatgtacaccgaagtcaagaacgcaaaaggtgaatgggtatttaagtctttgggtaaacctggatccatgcatttaagacctattgctactccttaccctgttaaggaatggttgcaaccaaaacgttataaggcacacttgatgggtaccacatatgtctatgacttcccagaattattccgccaagcatcgtcatcccaatggaaaaatttctctgcagatgttaagttaacagatgatttctttatttccaacgagttgattgaagatgaaaacggcgaattaactgaggtggaaagagaacctggtgccaacgctattggtatggttgcctttaagattactgtaaagactcctgaatatccaagaggccgtcaatttgttgttgttgctaacgatatcacattcaagatcggttcctttggtccacaagaagacgaattcttcaataaggttactgaatatgctagaaagcgtggtatcccaagaatttacttggctgcaaactcaggtgccagaattggtatggctgaagagattgttccactatttcaagttgcatggaatgatgctgccaatccggacaagggcttccaatacttatacttaacaagtgaaggtatggaaactttaaagaaatttgacaaagaaaattctgttctcactgaacgtactgttataaacggtgaagaaagatttgtcatcaagacaattattggttctgaagatgggttaggtgtcgaatgtctacgtggatctggtttaattgctggtgcaacgtcaagggcttaccacgatatcttcactatcaccttagtcacttgtagatccgtcggtatcggtgcttatttggttcgtttgggtcaaagagctattcaggtcgaaggccagccaattattttaactggtgctcctgcaatcaacaaaatgctgggtagagaagtttatacttctaacttacaattgggtggtactcaaatcatgtataacaacggtgtttcacatttgactgctgttgacgatttagctggtgtagagaagattgttgaatggatgtcttatgttccagccaagcgtaatatgccagttcctatcttggaaactaaagacacatgggatagaccagttgatttcactccaactaatgatgaaacttacgatgtaagatggatgattgaaggtcgtgagactgaaagtggatttgaatatggtttgtttgataaagggtctttctttgaaactttgtcaggatgggccaaaggtgttgtcgttggtagagcccgtcttggtggtattccactgggtgttattggtgttgaaacaagaactgtcgagaacttgattcctgctgatccagctaatccaaatagtgctgaaacattaattcaagaacctggtcaagtttggcatccaaactccgccttcaagactgctcaagctatcaatgactttaacaacggtgaacaattgccaatgatgattttggccaactggagaggtttctctggtggtcaacgtgatatgttcaacgaagtcttgaagtatggttcgtttattgttgacgcattggtggattacaaacaaccaattattatctatatcccacctaccggtgaactaagaggtggttcatgggttgttgtcgatccaactatcaacgctgaccaaatggaaatgtatgccgacgtcaacgctagagctggtgttttggaaccacaaggtatggttggtatcaagttccgtagagaaaaattgctggacaccatgaacagattggatgacaagtacagagaattgagatctcaattatccaacaagagtttggctccagaagtacatcagcaaatatccaagcaattagctgatcgtgagagagaactattgccaatttacggacaaatcagtcttcaatttgctgatttgcacgataggtcttcacgtatggtggccaagggtgttatttctaaggaactggaatggaccgaggcacgtcgtttcttcttctggagattgagaagaagattgaacgaagaatatttgattaaaaggttgagccatcaggtaggcgaagcatcaagattagaaaagatcgcaagaattagatcgtggtaccctgcttcagtggaccatgaagatgataggcaagtcgcaacatggattgaagaaaactacaaaactttggacgataaactaaagggtttgaaattagagtcattcgctcaagacttagctaaaaagatcagaagcgaccatgacaatgctattgatggattatctgaagttatcaagatgttatctaccgatgataaagaaaaattgttgaagactttgaaataa

SEQ ID NO: 138 - DNA, 1632 bp, synthetic construct
  source 1..1632: mol_type = other DNA
atgaaatgttctactttttctttttggtttgtttgtaaaattatttttttttttttttcttttaatattcaaacttctattgctaatccaagagaaaattttttgaaatgtttttctcaatatattccaaataatgctactaatttgaaattggtttatactcaaaataatccattgtatatgtctgttttgaattctactattcataatttgagatttacttctgatactactccaaaaccattggttattgttactccatctcatgtttctcatattcaaggtactattttgtgttctaaaaaagttggtttgcaaattagaactagatctggtggtcatgattctgaaggtatgtcttatatttctcaagttccatttgttattgttgatttgagaaatatgagatctattaaaattgatgttcattctcaaactgcttgggttgaagctggtgctactttgggtgaagtttattattgggttaatgaaaaaaatgaaaatttgtctttggctgctggttattgtccaactgtttgtgctggtggtcattttggtggtggtggttatggtccattgatgagaaattatggtttggctgctgataatattattgatgctcatttggttaatgttcatggtaaagttttggatagaaaatctatgggtgaagatttgttttgggctttgagaggtggtggtgctgaatcttttggtattattgttgcttggaaaattagattggttgctgttccaaaatctactatgttttctgttaaaaaaattatggaaattcatgaattggttaaattggttaataaatggcaaaatattgcttataaatatgataaagatttgttgttgatgactcattttattactagaaatattactgataatcaaggtaaaaataaaactgctattcatacttatttttcttctgtttttttgggtggtgttgattctttggttgatttgatgaataaatcttttccagaattgggtattaaaaaaactgattgtagacaattgtcttggattgatactattattttttattctggtgttgttaattatgatactgataattttaataaagaaattttgttggatagatctgctggtcaaaatggtgcttttaaaattaaattggattatgttaaaaaaccaattccagaatctgtttttgttcaaattttggaaaaattgtatgaagaagatattggtgctggtatgtatgctttgtatccatatggtggtattatggatgaaatttctgaatctgctattccatttccacatagagctggtattttgtatgaattgtggtatatttgttcttgggaaaaacaagaagataatgaaaaacatttgaattggattagaaatatttataattttatgactccatatgtttctaaaaatccaagattggcttatttgaattatagagatttggatattggtattaatgatccaaaaaatccaaataattatactcaagctagaatttggggtgaaaaatattttggtaaaaattttgatagattggttaaagttaaaactttggttgatccaaataatttttttagaaatgaacaatctattccaccattgccaagacatagacat

SEQ ID NO: 139 - DNA, 1635 bp, synthetic construct
  source 1..1635: mol_type = other DNA
atgaattgtagtactttctctttctggtttgtttgtaagattatatttttttttcttagtttcaatatacaaatttcaattgcaaaccctcaagaaaatttccttaagtgcttttcagaatatatccctaataatcctgcaaaccctaaattcatttatacacaacatgatcagttatatatgtctgtcttaaactctaccattcaaaatttgaggttcacgtctgatacaaccccaaagcctttagttatcgtgacaccctctaacgttagtcatattcaggctagtatcttatgttcaaaaaaagtgggtttacaaatcagaactaggtctggtggtcatgacgcggaaggtctgtcttacatatctcaggtgccgtttgcaatcgttgatctacgtaatatgcatacagttaaagtcgatattcactctcaaactgcatgggtcgaggctggtgccactctaggtgaagtttattactggatcaatgaaatgaacgagaatttttccttcccaggtggttattgtcctactgtgggtgtaggcggacacttttctggcggggggtatggtgctttgatgaggaactatggtttggccgccgataatataattgacgcccatcttgtaaacgtcgacgggaaggttctggaccgtaaatctatgggtgaagatttattctgggcgataagaggtggcgggggagagaactttggtattatcgcagcttgtaagattaagttagttgttgtcccctcaaaagcaacaattttttcagtgaagaagaacatggaaatccacggtttggtaaaactgtttaataaatggcagaatattgcctacaaatacgataaggatttgatgttgacaacacatttcagaactagaaatattactgacaaccacggaaagaacaagacaaccgtccatggatattttagttctattttcttaggcggagttgattcactagtagacttaatgaacaagtctttccccgaattgggaataaaaaaaaccgattgcaaggaattatcctggatagatacaacaatattctactctggagtcgttaattataatacggccaactttaagaaggaaatattattagatcgttccgcaggtaaaaagacagctttttccataaaattggactacgtcaaaaaattaattcctgagacagccatggtaaaaatattggaaaaattgtacgaagaggaggtaggcgtgggtatgtatgtgttatacccatacggtggtattatggatgaaatttctgagagcgctattcccttcccccatcgtgcaggtataatgtatgaattatggtacacagcaacatgggaaaaacaagaggataacgaaaagcatattaattgggtacgtagtgtgtacaactttacgacaccttacgtgtcccaaaatccaagattagcgtatttgaactatagagacttagatttaggtaaaacaaaccctgagtctccaaataattacacccaagccaggatttggggtgaaaaatacttcggcaaaaatttcaatagattggttaaggtaaaaactaaggcggatccaaacaatttttttagaaatgagcagagtattccgcccctgcctccaagacaccat

SEQ ID NO: 140 - DNA, 2160 bp, synthetic construct
  source 1..2160: mol_type = other DNA
atgggtaaaaattataaatctttggattctgttgttgcttctgattttattgctttgggtattacttctgaagttgctgaaactttgcatggtagattggctgaaattgtttgtaattatggtgctgctactccacaaacttggattaatattgctaatcatattttgtctccagatttgccattttctttgcatcaaatgttgttttatggttgttataaagattttggtccagctccaccagcttggattccagatccagaaaaagttaaatctactaatttgggtgctttgttggaaaaaagaggtaaagaatttttgggtgttaaatataaagatccaatttcttctttttctcattttcaagaattttctgttagaaatccagaagtttattggagaactgttttgatggatgaaatgaaaatttctttttctaaagatccagaatgtattttgagaagagatgatattaataatccaggtggttctgaatggttgccaggtggttatttgaattctgctaaaaattgtttgaatgttaattctaataaaaaattgaatgatactatgattgtttggagagatgaaggtaatgatgatttgccattgaataaattgactttggatcaattgagaaaaagagtttggttggttggttatgctttggaagaaatgggtttggaaaaaggttgtgctattgctattgatatgccaatgcatgttgatgctgttgttatttatttggctattgttttggctggttatgttgttgtttctattgctgattctttttctgctccagaaatttctactagattgagattgtctaaagctaaagctatttttactcaagatcatattattagaggtaaaaaaagaattccattgtattctagagttgttgaagctaaatctccaatggctattgttattccatgttctggttctaatattggtgctgaattgagagatggtgatatttcttgggattattttttggaaagagctaaagaatttaaaaattgtgaatttactgctagagaacaaccagttgatgcttatactaatattttgttttcttctggtactactggtgaaccaaaagctattccatggactcaagctactccattgaaagctgctgctgatggttggtctcatttggatattagaaaaggtgatgttattgtttggccaactaatttgggttggatgatgggtccatggttggtttatgcttctttgttgaatggtgcttctattgctttgtataatggttctccattggtttctggttttgctaaatttgttcaagatgctaaagttactatgttgggtgttgttccatctattgttagatcttggaaatctactaattgtgtttctggttatgattggtctactattagatgtttttcttcttctggtgaagcttctaatgttgatgaatatttgtggttgatgggtagagctaattataaaccagttattgaaatgtgtggtggtactgaaattggtggtgctttttctgctggttcttttttgcaagctcaatctttgtcttctttttcttctcaatgtatgggttgtactttgtatattttggataaaaatggttatccaatgccaaaaaataaaccaggtattggtgaattggctttgggtccagttatgtttggtgcttctaaaactttgttgaatggtaatcatcatgatgtttattttaaaggtatgccaactttgaatggtgaagttttgagaagacatggtgatatttttgaattgacttctaatggttattatcatgctcatggtagagctgatgatactatgaatattggtggtattaaaatttcttctattgaaattgaaagagtttgtaatgaagttgatgatagagtttttgaaactactgctattggtgttccaccattgggtggtggtccagaacaattggttattttttttgttttgaaagattctaatgatactactattgatttgaatcaattgagattgtcttttaatttgggtttgcaaaaaaaattgaatccattgtttaaagttactagagttgttccattgtcttctttgccaagaactgctactaataaaattatgagaagagttttgagacaacaattttctcattttgaa

SEQ ID NO: 141 - DNA, 4587 bp, synthetic construct
  source 1..4587: mol_type = other DNA
atgtctgctaaagctatttctgaacaaactggtaaagaattgttgtataaatttatttgtactacttctgctattcaaaatagatttaaatatgctagagttactccagatactgattgggctagattgttgcaagatcatccatggttgttgtctcaaaatttggttgttaaaccagatcaattgattaaaagaagaggtaaattgggtttggttggtgttaatttgactttggatggtgttaaatcttggttgaaaccaagattgggtcaagaagctactgttggtaaagctactggttttttgaaaaattttttgattgaaccatttgttccacattctcaagctgaagaattttatgtttgtatttatgctactagagaaggtgattatgttttgtttcatcatgaaggtggtgttgatgttggtgatgttgatgctaaagctcaaaaattgttggttggtgttgatgaaaaattgaatccagaagatattaaaaaacatttgttggttcatgctccagaagataaaaaagaaattttggcttcttttatttctggtttgtttaatttttatgaagatttgtattttacttatttggaaattaatccattggttgttactaaagatggtgtttatgttttggatttggctgctaaagttgatgctactgctgattatatttgtaaagttaaatggggtgatattgaatttccaccaccatttggtagagaagcttatccagaagaagcttatattgctgatttggatgctaaatctggtgcttctttgaaattgactttgttgaatccaaaaggtagaatttggactatggttgctggtggtggtgcttctgttgtttattctgatactatttgtgatttgggtggtgttaatgaattggctaattatggtgaatattctggtgctccatctgaacaacaaacttatgattatgctaaaactattttgtctttgatgactagagaaaaacatccagatggtaaaattttgattattggtggttctattgctaattttactaatgttgctgctacttttaaaggtattgttagagctattagagattatcaaggtccattgaaagaacatgaagttactatttttgttagaagaggtggtccaaattatcaagaaggtttgagagttatgggtgaagttggtaaaactactggtattccaattcatgtttttggtactgaaactcatatgactgctattgttggtatggctttgggtcatagaccaattccaaatcaaccaccaactgctgctcatactgctaattttttgttgaatgcttctggttctacttctactccagctccatctagaactgcttctttttctgaatctagagctgatgaagttgctccagctaaaaaagctaaaccagctatgccacaagattctgttccatctccaagatctttgcaaggtaaatctactactttgttttctagacatactaaagctattgtttggggtatgcaaactagagctgttcaaggtatgttggattttgattatgtttgttctagagatgaaccatctgttgctgctatggtttatccatttactggtgatcataaacaaaaattttattggggtcataaagaaattttgattccagtttttaaaaatatggctgatgctatgagaaaacatccagaagttgatgttttgattaattttgcttctttgagatctgcttatgattctactatggaaactatgaattatgctcaaattagaactattgctattattgctgaaggtattccagaagctttgactagaaaattgattaaaaaagctgatcaaaaaggtgttactattattggtccagctactgttggtggtattaaaccaggttgttttaaaattggtaatactggtggtatgttggataatattttggcttctaaattgtatagaccaggttctgttgcttatgtttctagatctggtggtatgtctaatgaattgaataatattatttctagaactactgatggtgtttatgaaggtgttgctattggtggtgatagatatccaggttctacttttatggatcatgttttgagatatcaagatactccaggtgttaaaatgattgttgttttgggtgaaattggtggtactgaagaatataaaatttgtagaggtattaaagaaggtagattgactaaaccaattgtttgttggtgtattggtacttgtgctactatgttttcttctgaagttcaatttggtcatgctggtgcttgtgctaatcaagcttctgaaactgctgttgctaaaaatcaagctttgaaagaagctggtgtttttgttccaagatcttttgatgaattgggtgaaattattcaatctgtttatgaagatttggttgctaatggtgttattgttccagctcaagaagttccaccaccaactgttccaatggattattcttgggctagagaattgggtttgattagaaaaccagcttcttttatgacttctatttgtgatgaaagaggtcaagaattgatttatgctggtatgccaattactgaagtttttaaagaagaaatgggtattggtggtgttttgggtttgttgtggtttcaaaaaagattgccaaaatattcttgtcaatttattgaaatgtgtttgatggttactgctgatcatggtccagctgtttctggtgctcataatactattatttgtgctagagctggtaaagatttggtttcttctttgacttctggtttgttgactattggtgatagatttggtggtgctttggatgctgctgctaaaatgttttctaaagcttttgattctggtattattccaatggaatttgttaataaaatgaaaaaagaaggtaaattgattatgggtattggtcatagagttaaatctattaataatccagatatgagagttcaaattttgaaagattatgttagacaacattttccagctactccattgttggattatgctttggaagttgaaaaaattactacttctaaaaaaccaaatttgattttgaatgttgatggtttgattggtgttgcttttgttgatatgttgagaaattgtggttcttttactagagaagaagctgatgaatatattgatattggtgctttgaatggtatttttgttttgggtagatctatgggttttattggtcattatttggatcaaaaaagattgaaacaaggtttgtatagacatccatgggatgatatttcttatgttttgccagaacatatgtctatgaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttcttcttattatcatcatcatcatcatcatttggaatctacttctttgtataaaaaagctggttctggttctaatttggttgctcaattggaaaatgaagttgcttctttggaaaatgaaaatgaaactttgaaaaaaaaaaatttgcataaaaaagatttgattgcttatttggaaaaagaaattgctaatttgagaaaaaaaattgaagaaggttctgctggttctgctgctggttctggtgaatttggttctgctgaagctgctgctaaagaagctgctgctaaagctggttctgctggttctgctgctggttctggtgaatttggttcttcttattatcatcatcatcatcatcatttggaatctacttctttgtataaaaaagctggttctggttctgctagaaatgcttatttgagaaaaaaaattgctagattgaaaaaagataatttgcaattggaaagagatgaacaaaatttggaaaaaattattgctaatttgagagatgaaattgctagattggaaaatgaagttgcttctcatgaacaa

SEQ ID NO: 142 - DNA, 2463 bp, synthetic construct
  source 1..2463: mol_type = other DNA
atgaaaaattgtgttattgtttctgctgttagaactgctattggttcttttaatggttctttggcttctacttctgctattgatttgggtgctactgttattaaagctgctattgaaagagctaaaattgattctcaacatgttgatgaagttattatgggtaatgttttgcaagctggtttgggtcaaaatccagctagacaagctttgttgaaatctggtttggctgaaactgtttgtggttttactgttaataaagtttgtggttctggtttgaaatctgttgctttggctgctcaagctattcaagctggtcaagctcaatctattgttgctggtggtatggaaaatatgtctttggctccatatttgttggatgctaaagctagatctggttatagattgggtgatggtcaagtttatgatgttattttgagagatggtttgatgtgtgctactcatggttatcatatgggtattactgctgaaaatgttgctaaagaatatggtattactagagaaatgcaagatgaattggctttgcattctcaaagaaaagctgctgctgctattgaatctggtgcttttactgctgaaattgttccagttaatgttgttactagaaaaaaaacttttgttttttctcaagatgaatttccaaaagctaattctactgctgaagctttgggtgctttgagaccagcttttgataaagctggtactgttactgctggtaatgcttctggtattaatgatggtgctgctgctttggttattatggaagaatctgctgctttggctgctggtttgactccattggctagaattaaatcttatgcttctggtggtgttccaccagctttgatgggtatgggtccagttccagctactcaaaaagctttgcaattggctggtttgcaattggctgatattgatttgattgaagctaatgaagcttttgctgctcaatttttggctgttggtaaaaatttgggttttgattctgaaaaagttaatgttaatggtggtgctattgctttgggtcatccaattggtgcttctggtgctagaattttggttactttgttgcatgctatgcaagctagagataaaactttgggtttggctactttgtgtattggtggtggtcaaggtattgctatggttattgaaagattgaataaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttcttcttattatcatcatcatcatcatcatttggaatctacttctttgtataaaaaagctggttctggttctaatgaagttactactttggaaaatgatgctgcttttattgaaaatgaaaatgcttatttggaaaaagaaattgctagattgagaaaagaaaaagctgctttgagaaatagattggctcataaaaaaggttctgctggttctgctgctggttctggtgaatttggttctgctgaagctgctgctaaagaagctgctgctaaagctggttctgctggttctgctgctggttctggtgaatttggttcttcttattatcatcatcatcatcatcatttggaatctacttctttgtataaaaaagctggttctggttctcaaaaagttgctgaattgaaaaatagagttgctgttaaattgaatagaaatgaacaattgaaaaataaagttgaagaattgaaaaatagaaatgcttatttgaaaaatgaattggctactttggaaaatgaagttgctagattggaaaatgatgttgctgaa

SEQ ID NO: 143 - DNA, 2328 bp, synthetic construct
  source 1..2328: mol_type = other DNA
atgaaaaaagtttgtgttattggtgctggtactatgggttctggtattgctcaagcttttgctgctaaaggttttgaagttgttttgagagatattaaagatgaatttgttgatagaggtttggattttattaataaaaatttgtctaaattggttaaaaaaggtaaaattgaagaagctactaaagttgaaattttgactagaatttctggtactgttgatttgaatatggctgctgattgtgatttggttattgaagctgctgttgaaagaatggatattaaaaaacaaatttttgctgatttggataatatttgtaaaccagaaactattttggcttctaatacttcttctttgtctattactgaagttgcttctgctactaaaagaccagataaagttattggtatgcatttttttaatccagctccagttatgaaattggttgaagttattagaggtattgctacttctcaagaaacttttgatgctgttaaagaaacttctattgctattggtaaagatccagttgaagttgctgaagctccaggttttgttgttaatagaattttgattccaatgattaatgaagctgttggtattttggctgaaggtattgcttctgttgaagatattgataaagctatgaaattgggtgctaatcatccaatgggtccattggaattgggtgattttattggtttggatatttgtttggctattatggatgttttgtattctgaaactggtgattctaaatatagaccacatactttgttgaaaaaatatgttagagctggttggttgggtagaaaatctggtaaaggtttttatgattattctaaaaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttctgaaaatttgtattttcaaggtgaaaatttgtattttcaaggtgattcttctgaatcttgttggaattgtggtagaaaagcttctgaaacttgttctggttgtaatactgctagatattgtggttctttttgtcaacataaagattgggaaaaacatcatcatatttgtggtcaaactttgcaagctcaacaaggttctgctggttctgctgctggttctggtgaatttggttctgctgaagctgctgctaaagaagctgctgctaaagctggttctgctggttctgctgctggttctggtgaatttggttctatggctgtttctgaatctcaattgaaaaaaatggtttctaaatataaatatagagatttgactgttagagaaactgttaatgttattactttgtataaagatttgaaaccagttttggattcttatgtttttaatgatggttcttctagagaattgatgaatttgactggtactattccagttccatatagaggtaatacttataatattccaatttgtttgtggttgttggatacttatccatataatccaccaatttgttttgttaaaccaacttcttctatgactattaaaactggtaaacatgttgatgctaatggtaaaatttatttgccatatttgcatgaatggaaacatccacaatctgatttgttgggtttgattcaagttatgattgttgtttttggtgatgaaccaccagttttttctagacca

SEQ ID NO: 144 - DNA, 2223 bp, synthetic construct
  source 1..2223: mol_type = other DNA
atggaattgaataatgttattttggaaaaagaaggtaaagttgctgttgttactattaatagaccaaaagctttgaatgctttgaattctgatactttgaaagaaatggattatgttattggtgaaattgaaaatgattctgaagttttggctgttattttgactggtgctggtgaaaaatcttttgttgctggtgctgatatttctgaaatgaaagaaatgaatactattgaaggtagaaaatttggtattttgggtaataaagtttttagaagattggaattgttggaaaaaccagttattgctgctgttaatggttttgctttgggtggtggttgtgaaattgctatgtcttgtgatattagaattgcttcttctaatgctagatttggtcaaccagaagttggtttgggtattactccaggttttggtggtactcaaagattgtctagattggttggtatgggtatggctaaacaattgatttttactgctcaaaatattaaagctgatgaagctttgagaattggtttggttaataaagttgttgaaccatctgaattgatgaatactgctaaagaaattgctaataaaattgtttctaatgctccagttgctgttaaattgtctaaacaagctattaatagaggtatgcaatgtgatattgatactgctttggcttttgaatctgaagcttttggtgaatgtttttctactgaagatcaaaaagatgctatgactgcttttattgaaaaaagaaaaattgaaggttttaaaaatagaaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttctggtccattgggttctccattgactgcttctatgttggcttctgctccaccacaagaacaaaaacaaatgttgggtgaaagattgtttccattgattcaagctatgcatccaactttggctggtaaaattactggtatgttgttggaaattgataattctgaattgttgcatatgttggaatctccagaatctttgagatctaaagttgatgaagctgttgctgttttgcaagctcatcaagctaaagaagctgctcaaaaagctggttctgctggttctgctgctggttctggtgaatttggttctgctgaagctgctgctaaagaagctgctgctaaagctggttctgctggttctgctgctggttctggtgaatttggttctaatactaatatgtctgttccaactgatggtgctgttactacttctcaaattccagcttctgaacaagaaactttggttagaccaaaaccattgttgttgaaattgttgaaatctgttggtgctcaaaaagatacttatactatgaaagaagttttgttttatttgggtcaatatattatgactaaaagattgtatgatgaaaaacaacaacatattgtttattgttctaatgatttgttgggtgatttgtttggtgttccatctttttctgttaaagaacatagaaaaatttatactatgatttatagaaatttggttgtt

SEQ ID NO: 145 - DNA, 2460 bp, synthetic construct
  source 1..2460: mol_type = other DNA
atgattgttaaaccaatggttagaaataatatttgtttgaatgctcatccacaaggttgtaaaaaaggtgttgaagatcaaattgaatatactaaaaaaagaattactgctgaagttaaagctggtgctaaagctccaaaaaatgttttggttttgggttgttctaatggttatggtttggcttctagaattactgctgcttttggttatggtgctgctactattggtgtttcttttgaaaaagctggttctgaaactaaatatggtactccaggttggtataataatttggcttttgatgaagctgctaaaagagaaggtttgtattctgttactattgatggtgatgctttttctgatgaaattaaagctcaagttattgaagaagctaaaaaaaaaggtattaaatttgatttgattgtttattctttggcttctccagttagaactgatccagatactggtattatgcataaatctgttttgaaaccatttggtaaaacttttactggtaaaactgttgatccatttactggtgaattgaaagaaatttctgctgaaccagctaatgatgaagaagctgctgctactgttaaagttatgggtggtgaagattgggaaagatggattaaacaattgtctaaagaaggtttgttggaagaaggttgtattactttggcttattcttatattggtccagaagctactcaagctttgtatagaaaaggtactattggtaaagctaaagaacatttggaagctactgctcatagattgaataaagaaaatccatctattagagcttttgtttctgttaataaaggtttggttactagagcttctgctgttattccagttattccattgtatttggcttctttgtttaaagttatgaaagaaaaaggtaatcatgaaggttgtattgaacaaattactagattgtatgctgaaagattgtatagaaaagatggtactattccagttgatgaagaaaatagaattagaattgatgattgggaattggaagaagatgttcaaaaagctgtttctgctttgatggaaaaagttactggtgaaaatgctgaatctttgactgatttggctggttatagacatgattttttggcttctaatggttttgatgttgaaggtattaattatgaagctgaagttgaaagatttgatagaattaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttcttcttattatcatcatcatcatcatcatttggaatctacttctttgtataaaaaagctggttctggttctaatttgttggctactttgagatctactgctgctgttttggaaaatgaaaatcatgttttggaaaaagaaaaagaaaaattgagaaaagaaaaagaacaattgttgaataaattggaagcttataaaggttctgctggttctgctgctggttctggtgaatttggttctgctgaagctgctgctaaagaagctgctgctaaagctggttctgctggttctgctgctggttctggtgaatttggttcttcttattatcatcatcatcatcatcatttggaatctacttctttgtataaaaaagctggttctggttctaaaagaattgcttatttgagaaaaaaaattgctgctttgaaaaaagataatgctaatttggaaaaagatattgctaatttggaaaatgaaattgaaagattgattaaagaaattaaaactttggaaaatgaagttgcttctcatgaacaa

SEQ ID NO: 146 - DNA, 2478 bp, synthetic construct
  source 1..2478: mol_type = other DNA
atgactagagaagttgttgttgtttctggtgttagaactgctattggtacttttggtggttctttgaaagatgttgctccagctgaattgggtgctttggttgttagagaagctttggctagagctcaagtttctggtgatgatgttggtcatgttgtttttggtaatgttattcaaactgaaccaagagatatgtatttgggtagagttgctgctgttaatggtggtgttactattaatgctccagctttgactgttaatagattgtgtggttctggtttgcaagctattgtttctgctgctcaaactattttgttgggtgatactgatgttgctattggtggtggtgctgaatctatgtctagagctccatatttggctccagctgctagatggggtgctagaatgggtgatgctggtttggttgatatgatgttgggtgctttgcatgatccatttcatagaattcatatgggtgttactgctgaaaatgttgctaaagaatatgatatttctagagctcaacaagatgaagctgctttggaatctcatagaagagcttctgctgctattaaagctggttattttaaagatcaaattgttccagttgtttctaaaggtagaaaaggtgatgttacttttgatactgatgaacatgttagacatgatgctactattgatgatatgactaaattgagaccagtttttgttaaagaaaatggtactgttactgctggtaatgcttctggtttgaatgatgctgctgctgctgttgttatgatggaaagagctgaagctgaaagaagaggtttgaaaccattggctagattggtttcttatggtcatgctggtgttgatccaaaagctatgggtattggtccagttccagctactaaaattgctttggaaagagctggtttgcaagtttctgatttggatgttattgaagctaatgaagcttttgctgctcaagcttgtgctgttactaaagctttgggtttggatccagctaaagttaatccaaatggttctggtatttctttgggtcatccaattggtgctactggtgctttgattactgttaaagctttgcatgaattgaatagagttcaaggtagatatgctttggttactatgtgtattggtggtggtcaaggtattgctgctatttttgaaagaattaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttctgatgttatgtgggaatataaatgggaaaatactggtgatgctgaattgtatggtccatttacttctgctcaaatgcaaacttgggtttctgaaggttattttccagatggtgtttattgtagaaaattggatccaccaggtggtcaattttataattctaaaagaattgattttgatttgtatactggttctgctggttctgctgctggttctggtgaatttggttctgctgaagctgctgctaaagaagctgctgctaaagctggttctgctggttctgctgctggttctggtgaatttggttctgaatctgattctgttgaatttaataatgctatttcttatgttaataaaattaaaactagatttttggatcatccagaaatttatagatcttttttggaaattttgcatacttatcaaaaagaacaattgcatactaaaggtagaccatttagaggtatgtctgaagaagaagtttttactgaagttgctaatttgtttagaggtcaagaagatttgttgtctgaatttggtcaatttttgccagaagctaaaaga

SEQ ID NO: 147 - DNA, 2550 bp, synthetic construct
  source 1..2550: mol_type = other DNA
atgaaactctcaactaaactttgttggtgtggtattaaaggaagacttaggccgcaaaagcaacaacaattacacaatacaaacttgcaaatgactgaactaaaaaaacaaaagaccgctgaacaaaaaaccagacctcaaaatgtcggtattaaaggtatccaaatttacatcccaactcaatgtgtcaaccaatctgagctagagaaatttgatggcgtttctcaaggtaaatacacaattggtctgggccaaaccaacatgtcttttgtcaatgacagagaagatatctactcgatgtccctaactgttttgtctaagttgatcaagagttacaacatcgacaccaacaaaattggtagattagaagtcggtactgaaactctgattgacaagtccaagtctgtcaagtctgtcttgatgcaattgtttggtgaaaacactgacgtcgaaggtattgacacgcttaatgcctgttacggtggtaccaacgcgttgttcaactctttgaactggattgaatctaacgcatgggatggtagagacgccattgtagtttgcggtgatattgccatctacgataagggtgccgcaagaccaaccggtggtgccggtactgttgctatgtggatcggtcctgatgctccaattgtatttgactctgtaagagcttcttacatggaacacgcctacgatttttacaagccagatttcaccagcgaatatccttacgtcgatggtcatttttcattaacttgttacgtcaaggctcttgatcaagtttacaagagttattccaagaaggctatttctaaagggttggttagcgatcccgctggttcggatgctttgaacgttttgaaatatttcgactacaacgttttccatgttccaacctgtaaattggtcacaaaatcatacggtagattactatataacgatttcagagccaatcctcaattgttcccagaagttgacgccgaattagctactcgcgattatgacgaatctttaaccgataagaacattgaaaaaacttttgttaatgttgctaagccattccacaaagagagagttgcccaatctttgattgttccaacaaacacaggtaacatgtacaccgcatctgtttatgccgcctttgcatctctattaaactatgttggatctgacgacttacaaggcaagcgtgttggtttattttcttacggttccggtttagctgcatctctatattcttgcaaaattgttggtgacgtccaacatattatcaaggaattagatattactaacaaattagccaagagaatcaccgaaactccaaaggattacgaagctgccatcgaattgagagaaaatgcccatttgaagaagaacttcaaacctcaaggttccattgagcatttgcaaagtggtgtttactacttgaccaacatcgatgacaaatttagaagatcttacgatgttaaaaaataaaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttctttgggtccattgccaccaggttgggaagttagatctactgtttctggtagaatttattttgttgatcataataatagaactactcaatttactgatccaagattgcatggttctgctggttctgctgctggttctggtgaatttggttctgctgaagctgctgctaaagaagctgctgctaaagctggttctgctggttctgctgctggttctggtgaatttggttctggtgctatgggtccattgccaccaggttgggaaaaaagaactgattctaatggtagagtttattttgttaatcataatactagaattactcaatgggaagatccaagatct

SEQ ID NO: 148 - DNA, 2478 bp, synthetic construct
  source 1..2478: mol_type = other DNA
atggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttcttcttattatcatcatcatcatcatcatttggaatctacttctttgtataaaaaagctggttctgaattttttagaagagaaagaaataaaatggctgctgctaaatgtagaaatagaagaagagaattgactgatactttgcaagctgaaactgatcaattggaagatgaaaaatctgctttgcaaactgaaattgctaatttgttgaaagaaaaagaaaaattggaatttattttggctgctcatagaccagcttgtaaaattccagatgatttgggttttccagaagaaatgtctttggaaggttctgctggttctgctgctggttctggtgaatttggttctgctgaagctgctgctaaagaagctgctgctaaagctggttctgctggttctgctgctggttctggtgaatttggttcttcttattatcatcatcatcatcatcatttggaatctacttctttgtataaaaaagctggttctggttctcaaaaagttgaatctttgaaacaaaaaattgaagaattgaaacaaagaaaagctcaattgaaaaatgatattgctaatttggaaaaagaaattgcttatgctgaaact

SEQ ID NO: 149 - DNA, 3141 bp, synthetic construct
  source 1..3141: mol_type = other DNA
atgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataaaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttctatggaaccagctatggaaccagaaactttggaagctagaattaatagagctactaatccattgaataaagaattggattgggcttctattaatggtttttgtgaacaattgaatgaagattttgaaggtccaccattggctactagattgttggctcataaaattcaatctccacaagaatgggaagctattcaagctttgactgttttggaaacttgtatgaaatcttgtggtaaaagatttcatgatgaagttggtaaatttagatttttgaatgaattgattaaagttgtttctccaaaatatttgggttctagaacttctgaaaaagttaaaaataaaattttggaattgttgtattcttggactgttggtttgccagaagaagttaaaattgctgaagcttatcaaatgttgaaaaaacaaggtattgttaaatctggttctgctggttctgctgctggttctggtgaatttggttctgctgaagctgctgctaaagaagctgctgctaaagctggttctgctggttctgctgctggttctggtgaatttggttctggtgctatgggttctatggctgaagctgaaggtgaatctttggaatcttggttgaataaagctactaatccatctaatagacaagaagattgggaatatattattggtttttgtgatcaaattaataaagaattggaaggtccacaaattgctgttagattgttggctcataaaattcaatctccacaagaatgggaagctttgcaagctttgactgttttggaagcttgtatgaaaaattgtggtagaagatttcataatgaagttggtaaatttagatttttgaatgaattgattaaagttgtttctccaaaatatttgggtgatagagtttctgaaaaagttaaaactaaagttattgaattgttgtattcttggactatggctttgccagaagaagctaaaattaaagatgcttatcatatgttgaaaagacaaggtattgttcaatctgatccaccaattccagttgatagaactttgattccatctccaccaccaagaccaaaaaat

SEQ ID NO: 150 - DNA, 2604 bp, synthetic construct
  source 1..2604: mol_type = other DNA
atgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataaaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttcttcttattatcatcatcatcatcatcatttggaatctacttctttgtataaaaaagctggttctggttctcaaaaagttgaagaattgaaaaataaaattgctgaattggaaaatagaaatgctgttaaaaaaaatagagttgctcatttgaaacaagaaattgcttatttgaaagatgaattggctgctcatgaatttgaaggttctgctggttctgctgctggttctggtgaatttggttctgctgaagctgctgctaaagaagctgctgctaaagctggttctgctggttctgctgctggttctggtgaatttggttcttcttattatcatcatcatcatcatcatttggaatctacttctttgtataaaaaagctggttctggttcttttgaaaatgttactcatgaatttattttggctactttggaaaatgaaaatgctaaattgagaagattggaagctaaattggaaagagaattggctagattgagaaatgaagttgcttggttg

SEQ ID NO: 151 - DNA, 3027 bp, synthetic construct
  source 1..3027: mol_type = other DNA
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataaaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttctgctatggctgatttggaacaaaaagttttggaaatggaagcttctacttatgatggtgtttttatttggaaaatttctgattttccaagaaaaagacaagaagctgttgctggtagaattccagctattttttctccagctttttatacttctagatatggttataaaatgtgtttgagaatttatttgaatggtgatggtactggtagaggtactcatttgtctttgttttttgttgttatgaaaggtccaaatgatgctttgttgagatggccatttaatcaaaaagttactttgatgttgttggatcaaaataatagagaacatgttattgatgcttttagaccagatgttacttcttcttcttttcaaagaccagttaatgatatgaatattgcttctggttgtccattgttttgtccagtttctaaaatggaagctaaaaattcttatgttagagatgatgctatttttattaaagctattgttgatttgactggtttgggttctgctggttctgctgctggttctggtgaatttggttctgctgaagctgctgctaaagaagctgctgctaaagctggttctgctggttctgctgctggttctggtgaatttggttctgcttctattaaattgcaatcttctgatggtgaaatttttgaagttgatgttgaaattgctaaacaatctgttactattaaaactatgttggaagatttgggtatggatgatgaaggtgatgatgatccagttccattgccaaatgttaatgctgctattttgaaaaaagttattcaatggtgtactcatcataaagatgatccaccaccaccagaagatgatgaaaataaagaaaaaagaactgatgatattccagtttgggatcaagaatttttgaaagttgatcaaggtactttgtttgaattgattttggctgctaattatttggatattaaaggtttgttggatgttacttgtaaaactgttgctaatatgattaaaggtaaaactccagaagaaattagaaaaacttttaatattaaaaatgattttactgaagaagaagaagctcaagttagaaaagaaaatcaatggtgt

SEQ ID NO: 152 - DNA, 2154 bp, synthetic construct
  source 1..2154: mol_type = other DNA
atgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataaaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttcttcttattatcatcatcatcatcatcatttggaatctacttctttgtataaaaaagctggttctggttctaatactgttaaagaattgaaaaattatattcaagaattggaagaaagaaatgctgaattgaaaaatttgaaagaacatttgaaatttgctaaagctgaattggaatttgaattggctgctcataaatttgaaggttctgctggttctgctgctggttctggtgaatttggttctgctgaagctgctgctaaagaagctgctgctaaagctggttctgctggttctgctgctggttctggtgaatttggttcttcttattatcatcatcatcatcatcatttggaatctacttctttgtataaaaaagctggttctggttctcaaaaagttgctcaattgaaaaatagagttgcttataaattgaaagaaaatgctaaattggaaaatattgttgctagattggaaaatgataatgctaatttggaaaaagatattgctaatttggaaaaagatattgctaatttggaaagagatgttgctaga

SEQ ID NO: 153 - DNA, 2448 bp, synthetic construct
  source 1..2448: mol_type = other DNA
atggcttcagaaaaggaaataagaagagaaagattcttgaacgtattcccaaagttagttgaagaattgaacgctagtttgttagcttatggtatgcctaaagaagcctgcgattggtatgctcactctttaaactacaatactccaggtggtaaattgaatagaggtttgagtgtagttgatacttatgctatcttgtctaacaaaaccgttgaacaattaggtcaagaagaatacgaaaaggtcgctatcttgggttggtgtattgaattgttgcaagcatactttttggttgccgatgacatgatggataagtctataacaagaagaggtcaaccatgctggtacaaagttccagaagttggtgaaatagccataaatgatgcttttatgttggaagccgctatctataaattgttgaagtcacatttcagaaacgaaaagtactacatcgatattaccgaattattccacgaagttactttccaaacagaattgggtcaattgatggatttgataactgcacctgaagataaagttgacttgtcaaagttttccttgaagaaacattcattcatcgtcacctttgaaactgcttattactccttctatttgccagtcgccttggctatgtacgtagctggtattactgatgaaaaagacttgaagcaagcaagagatgttttgatacctttgggtgaatacttccaaatccaagatgactacttagactgtttcggtactccagaacaaataggtaaaatcggtacagatattcaagacaataagtgcagttgggttattaacaaggctttggaattagcatctgccgaacaaagaaagactttggatgaaaactacggtaaaaaggactcagttgctgaagcaaagtgtaagaaaatttttaatgatttgaagattgaacaattgtaccatgaatacgaagaatccatcgctaaagacttaaaggcaaagattagtcaagttgatgaatcaagaggttttaaagccgacgttttgacagctttcttgaataaggtctacaagagatcaaagtagaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttctttgtgtactatgaaaaaaggtccatctggttatggttttaatttgcattctgataaatctaaaccaggtcaatttattagatctgttgatccagattctccagctgaagcttctggtttgagagctcaagatagaattgttgaagttaatggtgtttgtatggaaggtaaacaacatggtgatgttgtttctgctattagagctggtggtgatgaaactaaattgttggttgttgatagagaaggttctgctggttctgctgctggttctggtgaatttggttctgctgaagctgctgctaaagaagctgctgctaaagctggttctgctggttctgctgctggttctggtgaatttggttcttcttctggtgctattatttatactgttgaattgaaaagatatggtggtccattgggtattactatttctggtactgaagaaccatttgatccaattattatttcttctttgactaaaggtggtttggctgaaagaactggtgctattcatattggtgatagaattttggctattaattcttcttctttgaaaggtaaaccattgtctgaagctattcatttgttgcaaatggctggtgaaactgttactttgaaaattaaaaaacaaactgatgctcaaccagcttcttct

SEQ ID NO: 154 - DNA, 2649 bp, synthetic construct
  source 1..2649: mol_type = other DNA
atgaatcatttgagagctgaaggtccagcttctgttttggctattggtactgctaatccagaaaatattttgttgcaagatgaatttccagattattattttagagttactaaatctgaacatatgactcaattgaaagaaaaatttagaaaaatttgtgataaatctatgattagaaaaagaaattgttttttgaatgaagaacatttgaaacaaaatccaagattggttgaacatgaaatgcaaactttggatgctagacaagatatgttggttgttgaagttccaaaattgggtaaagatgcttgtgctaaagctattaaagaatggggtcaaccaaaatctaaaattactcatttgatttttacttctgcttctactactgatatgccaggtgctgattatcattgtgctaaattgttgggtttgtctccatctgttaaaagagttatgatgtatcaattgggttgttatggtggtggtactgttttgagaattgctaaagatattgctgaaaataataaaggtgctagagttttggctgtttgttgtgatattatggcttgtttgtttagaggtccatctgaatctgatttggaattgttggttggtcaagctatttttggtgatggtgctgctgctgttattgttggtgctgaaccagatgaatctgttggtgaaagaccaatttttgaattggtttctactggtcaaactattttgccaaattctgaaggtactattggtggtcatattagagaagctggtttgatttttgatttgcataaagatgttccaatgttgatttctaataatattgaaaaatgtttgattgaagcttttactccaattggtatttctgattggaattctattttttggattactcatccaggtggtaaagctattttggataaagttgaagaaaaattgcatttgaaatctgataaatttgttgattctagacatgttttgtctgaacatggtaatatgtcttcttctactgttttgtttgttatggatgaattgagaaaaagatctttggaagaaggtaaatctactactggtgatggttttgaatggggtgttttgtttggttttggtccaggtttgactgttgaaagagttgttgttagatctgttccaattaaatataaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttctggtaataatttggaaacttatgaatggtataataaatctatttctagagataaagctgaaaaattgttgttggatactggtaaagaaggtgcttttatggttagagattctagaactccaggtacttatactgtttctgtttttactaaagctattatttctgaaaatccatgtattaaacattatcatattaaagaaactaatgattctccaaaaagatattatgttgctgaaaaatatgtttttgattctattccattgttgattcaatatcatcaatataatggtggtggtttggttactagattgagatatccagtttgtggtggttctgctggttctgctgctggttctggtgaatttggttctgctgaagctgctgctaaagaagctgctgctaaagctggttctgctggttctgctgctggttctggtgaatttggttctggttctcatccatggttttttggtaaaattccaagagctaaagctgaagaaatgttgtctaaacaaagacatgatggtgcttttttgattagagaatctgaatctgctccaggtgatttttctttgtctgttaaatttggtaatgatgttcaacattttaaagttttgagagatggtgctggtaaatattttttgtgggttgttaaatttaattctttgaatgaattggttgattatcatagatctacttctgtttctagaaatcaacaaatttttttgagagatattgaacaagttccacaacaaccaact

SEQ ID NO: 155 - DNA, 1995 bp, synthetic construct
  source 1..1995: mol_type = other DNA
atggctgttaaacatttgattgttttgaaatttaaagatgaaattactgaagctcaaaaagaagaattttttaaaacttatgttaatttggttaatattattccagctatgaaagatgtttattggggtaaagatgttactcaaaaaaataaagaagaaggttatactcatattgttgaagttacttttgaatctgttgaaactattcaagattatattattcatccagctcatgttggttttggtgatgtttatagatctttttgggaaaaattgttgatttttgattatactccaagaaaaaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttctggtcaagatagatctgaagctactttgattaaaagatttaaaggtgaaggtgttagatataaagctaaattgattggtattgatgaagtttctgctgctagaggtgataaattgtgtcaagattctatgatgaaattgaaaggtgttgttgctggtgctagatctaaaggtgaacataaacaaaaaatttttttgactatttcttttggtggtattaaaatttttgatgaaaaaactggtgctttgcaacatcatcatgctgttcatgaaatttcttatattgctaaagatattactgatcatagagcttttggttatgtttgtggtaaagaaggtaatcatagatttgttgctattaaaactgctcaagctgctgaaccagttattttggatttgagagatttgtttcaattgatttatgaattgaaacaaagagaagaattggaaaaaaaagctggttctgctggttctgctgctggttctggtgaatttggttctgctgaagctgctgctaaagaagctgctgctaaagctggttctgctggttctgctgctggttctggtgaatttggttctggttctcatatgggttctcaattttgggttacttctcaaaaaactgaagcttctgaaagatgtggtttgcaaggttcttatattttgagagttgaagctgaaaaattgactttgttgactttgggtgctcaatctcaaattttggaaccattgttgttttggccatatactttgttgagaagatatggtagagataaagttatgttttcttttgaagctggtagaagatgtccatctggtccaggtacttttacttttcaaacttctcaaggtaatgatatttttcaagctgttgaagctgctattcaacaacaaaaagctcaaggtaaagttggtcaagctcaagatattttgagattggaacatcatcatcatcatcat

SEQ ID NO: 156 - DNA, 2391 bp, synthetic construct
  source 1..2391: mol_type = other DNA
atgggtttgtcttctgtttgtactttttcttttcaaactaattatcatactttgttgaatccacataataataatccaaaaacttctttgttgtgttatagacatccaaaaactccaattaaatattcttataataattttccatctaaacattgttctactaaatcttttcatttgcaaaataaatgttctgaatctttgtctattgctaaaaattctattagagctgctactactaatcaaactgaaccaccagaatctgataatcattctgttgctactaaaattttgaattttggtaaagcttgttggaaattgcaaagaccatatactattattgcttttacttcttgtgcttgtggtttgtttggtaaagaattgttgcataatactaatttgatttcttggtctttgatgtttaaagcttttttttttttggttgctattttgtgtattgcttcttttactactactattaatcaaatttatgatttgcatattgatagaattaataaaccagatttgccattggcttctggtgaaatttctgttaatactgcttggattatgtctattattgttgctttgtttggtttgattattactattaaaatgaaaggtggtccattgtatatttttggttattgttttggtatttttggtggtattgtttattctgttccaccatttagatggaaacaaaatccatctactgcttttttgttgaattttttggctcatattattactaattttactttttattatgcttctagagctgctttgggtttgccatttgaattgagaccatcttttacttttttgttggcttttatgaaatctatgggttctgctttggctttgattaaagatgcttctgatgttgaaggtgatactaaatttggtatttctactttggcttctaaatatggttctagaaatttgactttgttttgttctggtattgttttgttgtcttatgttgctgctattttggctggtattatttggccacaagcttttaattctaatgttatgttgttgtctcatgctattttggctttttggttgattttgcaaactagagattttgctttgactaattatgatccagaagctggtagaagattttatgaatttatgtggaaattgtattatgctgaatatttggtttatgtttttattaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttctgctgaatatgttagagctttgtttgattttaatggtaatgatgaagaagatttgccatttaaaaaaggtgatattttgagaattagagataaaccagaagaacaatggtggaatgctgaagattctgaaggtaaaagaggtatgattccagttccatatgttgaaaaatatggttctgctggttctgctgctggttctggtgaatttggttctgctgaagctgctgctaaagaagctgctgctaaagctggttctgctggttctgctgctggttctggtgaatttggttctttgattaaacatatgagagctgaagctttgtttgattttactggtaattctaaattggaattgaattttaaagctggtgatgttatttttttgttgtctagaattaataaagattggttggaaggtactgttagaggtgctactggtatttttccattgtcttttgttaaaattttgaaa

SEQ ID NO: 157 - DNA, 8226 bp, synthetic construct
  source 1..8226: mol_type = other DNA
atgagcgaagaaagcttattcgagtcttctccacagaagatggagtacgaaattacaaactactcagaaagacatacagaacttccaggtcatttcattggcctcaatacagtagataaactagaggagtccccgttaagggactttgttaagagtcacggtggtcacacggtcatatccaagatcctgatagcaaataatggtattgccgccgtgaaagaaattagatccgtcagaaaatgggcatacgagacgttcggcgatgacagaaccgtccaattcgtcgccatggccaccccagaagatctggaggccaacgcagaatatatccgtatggccgatcaatacattgaagtgccaggtggtactaataataacaactacgctaacgtagacttgatcgtagacatcgccgaaagagcagacgtagacgccgtatgggctggctggggtcacgcctccgagaatccactattgcctgaaaaattgtcccagtctaagaggaaagtcatctttattgggcctccaggtaacgccatgaggtctttaggtgataaaatctcctctaccattgtcgctcaaagtgctaaagtcccatgtattccatggtctggtaccggtgttgacaccgttcacgtggacgagaaaaccggtctggtctctgtcgacgatgacatctatcaaaagggttgttgtacctctcctgaagatggtttacaaaaggccaagcgtattggttttcctgtcatgattaaggcatccgaaggtggtggtggtaaaggtatcagacaagttgaacgtgaagaagatttcatcgctttataccaccaggcagccaacgaaattccaggctcccccattttcatcatgaagttggccggtagagcgcgtcacttggaagttcaactgctagcagatcagtacggtacaaatatttccttgttcggtagagactgttccgttcagagacgtcatcaaaaaattatcgaagaagcaccagttacaattgccaaggctgaaacatttcacgagatggaaaaggctgccgtcagactggggaaactagtcggttatgtctctgccggtaccgtggagtatctatattctcatgatgatggaaaattctactttttagaattgaacccaagattacaagtcgagcatccaacaacggaaatggtctccggtgttaacttacctgcagctcaattacaaatcgctatgggtatccctatgcatagaataagtgacattagaactttatatggtatgaatcctcattctgcctcagaaatcgatttcgaattcaaaactcaagatgccaccaagaaacaaagaagacctattccaaagggtcattgtaccgcttgtcgtatcacatcagaagatccaaacgatggattcaagccatcgggtggtactttgcatgaactaaacttccgttcttcctctaatgtttggggttacttctccgtgggtaacaatggtaatattcactccttttcggactctcagttcggccatatttttgcttttggtgaaaatagacaagcttccaggaaacacatggttgttgccctgaaggaattgtccattaggggtgatttcagaactactgtggaatacttgatcaaacttttggaaactgaagatttcgaggataacactattaccaccggttggttggacgatttgattactcataaaatgaccgctgaaaagcctgatccaactcttgccgtcatttgcggtgccgctacaaaggctttcttagcatctgaagaagcccgccacaagtatatcgaatccttacaaaagggacaagttctatctaaagacctactgcaaactatgttccctgtagattttatccatgagggtaaaagatacaagttcaccgtagctaaatccggtaatgaccgttacacattatttatcaatggttctaaatgtgatatcatactgcgtcaactatctgatggtggtcttttgattgccataggcggtaaatcgcataccatctattggaaagaagaagttgctgctacaagattatccgttgactctatgactactttgttggaagttgaaaacgatccaacccagttgcgtactccatcccctggtaaattggttaaattcttggtggaaaatggtgaacacattatcaagggccaaccatatgcagaaattgaagttatgaaaatgcaaatgcctttggtttctcaagaaaatggtatcgtccagttattaaagcaacctggttctaccattgttgcaggtgatatcatggctattatgactcttgacgatccatccaaggtcaagcacgctctaccatttgaaggtatgctgccagattttggttctccagttatcgaaggaaccaaacctgcctataaattcaagtcattagtgtctactttggaaaacattttgaagggttatgacaaccaagttattatgaacgcttccttgcaacaattgatagaggttttgagaaatccaaaactgccttactcagaatggaaactacacatctctgctttacattcaagattgcctgctaagctagatgaacaaatggaagagttagttgcacgttctttgagacgtggtgctgttttcccagctagacaattaagtaaattgattgatatggccgtgaagaatcctgaatacaaccccgacaaattgctgggcgccgtcgtggaaccattggcggatattgctcataagtactctaacgggttagaagcccatgaacattctatatttgtccatttcttggaagaatattacgaagttgaaaagttattcaatggtccaaatgttcgtgaggaaaatatcattctgaaattgcgtgatgaaaaccctaaagatctagataaagttgcgctaactgttttgtctcattcgaaagtttcagcgaagaataacctgatcctagctatcttgaaacattatcaaccattgtgcaagttatcttctaaagtttctgccattttctctactcctctacaacatattgttgaactagaatctaaggctaccgctaaggtcgctctacaagcaagagaaattttgattcaaggcgctttaccttcggtcaaggaaagaactgaacaaattgaacatatcttaaaatcctctgttgtgaaggttgcctatggctcatccaatccaaagcgctctgaaccagatttgaatatcttgaaggacttgatcgattctaattacgttgtgttcgatgttttacttcaattcctaacccatcaagacccagttgtgactgctgcagctgctcaagtctatattcgtcgtgcttatcgtgcttacaccataggagatattagagttcacgaaggtgtcacagttccaattgttgaatggaaattccaactaccttcagctgcgttctccacctttccaactgttaaatctaaaatgggtatgaacagggctgtttctgtttcagatttgtcatatgttgcaaacagtcagtcatctccgttaagagaaggtattttgatggctgtggatcatttagatgatgttgatgaaattttgtcacaaagtttggaagttattcctcgtcaccaatcttcttctaacggacctgctcctgatcgttctggtagctccgcatcgttgagtaatgttgctaatgtttgtgttgcttctacagaaggtttcgaatctgaagaggaaattttggtaaggttgagagaaattttggatttgaataagcaggaattaatcaatgcttctatccgtcgtatcacatttatgttcggttttaaagatgggtcttatccaaagtattatacttttaacggtccaaattataacgaaaatgaaacaattcgtcacattgagccggctttggccttccaactggaattaggaagattgtccaacttcaacattaaaccaattttcactgataatagaaacatccatgtctacgaagctgttagtaagacttctccattggataagagattctttacaagaggtattattagaacgggtcatatccgtgatgacatttctattcaagaatatctgacttctgaagctaacagattgatgagtgatatattggataatttagaagtcaccgacacttcaaattctgatttgaatcatatcttcatcaacttcattgcggtgtttgatatctctccagaagatgtcgaagccgccttcggtggtttcttagaaagatttggtaagagattgttgagattgcgtgtttcttctgccgaaattagaatcatcatcaaagatcctcaaacaggtgccccagtaccattgcgtgccttgatcaataacgtttctggttatgttatcaaaacagaaatgtacaccgaagtcaagaacgcaaaaggtgaatgggtatttaagtctttgggtaaacctggatccatgcatttaagacctattgctactccttaccctgttaaggaatggttgcaaccaaaacgttataaggcacacttgatgggtaccacatatgtctatgacttcccagaattattccgccaagcatcgtcatcccaatggaaaaatttctctgcagatgttaagttaacagatgatttctttatttccaacgagttgattgaagatgaaaacggcgaattaactgaggtggaaagagaacctggtgccaacgctattggtatggttgcctttaagattactgtaaagactcctgaatatccaagaggccgtcaatttgttgttgttgctaacgatatcacattcaagatcggttcctttggtccacaagaagacgaattcttcaataaggttactgaatatgctagaaagcgtggtatcccaagaatttacttggctgcaaactcaggtgccagaattggtatggctgaagagattgttccactatttcaagttgcatggaatgatgctgccaatccggacaagggcttccaatacttatacttaacaagtgaaggtatggaaactttaaagaaatttgacaaagaaaattctgttctcactgaacgtactgttataaacggtgaagaaagatttgtcatcaagacaattattggttctgaagatgggttaggtgtcgaatgtctacgtggatctggtttaattgctggtgcaacgtcaagggcttaccacgatatcttcactatcaccttagtcacttgtagatccgtcggtatcggtgcttatttggttcgtttgggtcaaagagctattcaggtcgaaggccagccaattattttaactggtgctcctgcaatcaacaaaatgctgggtagagaagtttatacttctaacttacaattgggtggtactcaaatcatgtataacaacggtgtttcacatttgactgctgttgacgatttagctggtgtagagaagattgttgaatggatgtcttatgttccagccaagcgtaatatgccagttcctatcttggaaactaaagacacatgggatagaccagttgatttcactccaactaatgatgaaacttacgatgtaagatggatgattgaaggtcgtgagactgaaagtggatttgaatatggtttgtttgataaagggtctttctttgaaactttgtcaggatgggccaaaggtgttgtcgttggtagagcccgtcttggtggtattccactgggtgttattggtgttgaaacaagaactgtcgagaacttgattcctgctgatccagctaatccaaatagtgctgaaacattaattcaagaacctggtcaagtttggcatccaaactccgccttcaagactgctcaagctatcaatgactttaacaacggtgaacaattgccaatgatgattttggccaactggagaggtttctctggtggtcaacgtgatatgttcaacgaagtcttgaagtatggttcgtttattgttgacgcattggtggattacaaacaaccaattattatctatatcccacctaccggtgaactaagaggtggttcatgggttgttgtcgatccaactatcaacgctgaccaaatggaaatgtatgccgacgtcaacgctagagctggtgttttggaaccacaaggtatggttggtatcaagttccgtagagaaaaattgctggacaccatgaacagattggatgacaagtacagagaattgagatctcaattatccaacaagagtttggctccagaagtacatcagcaaatatccaagcaattagctgatcgtgagagagaactattgccaatttacggacaaatcagtcttcaatttgctgatttgcacgataggtcttcacgtatggtggccaagggtgttatttctaaggaactggaatggaccgaggcacgtcgtttcttcttctggagattgagaagaagattgaacgaagaatatttgattaaaaggttgagccatcaggtaggcgaagcatcaagattagaaaagatcgcaagaattagatcgtggtaccctgcttcagtggaccatgaagatgataggcaagtcgcaacatggattgaagaaaactacaaaactttggacgataaactaaagggtttgaaattagagtcattcgctcaagacttagctaaaaagatcagaagcgaccatgacaatgctattgatggattatctgaagttatcaagatgttatctaccgatgataaagaaaaattgttgaagactttgaaataaaaattgtctggtggtggtggttctggtggtggtggttctggtggtggtggttctgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaatattatcaaaaagctttggaattggatccaaataatgctgaagcttggtataatttgggtaatgcttattataaacaaggtgattatcaaaaagctattgaagattatcaaaaagctttggaattggatccaaataatttgcaagctgaagcttggaaaaatttgggtaatgcttattataaacaaggtgattatcaaaaagctattgaatattatcaaaaagctttggaattggatccaaataatgcttctgcttggtataatttgggtaatgcttattataaacaaggtgattatcaaaaagctattgaatattatcaaaaagctttggaattggatccaaataatgctaaagcttggtatagaagaggtaatgcttattataaacaaggtgattatcaaaaagctattgaagattatcaaaaagctttggaattggatccaaataatagatctagatctgctggtggtggtggttctggtggtggtggttctggtggtggtggtgcttctggttctcatatgagattgggtgctcaatctattcaaccaactgctaatttggatagaactgatgatttggtttatttgaatgttatggaattggttagagctgttttggaattgaaaaatgaattggctcaattgccaccagaaggttatgttgttgttgttaaaaatgttggtttgactttgagaaaattgattggttctgttgatgatttgttgccatctttgccatcttcttctagaactgaaattgaaggtactcaaaaattgttgaataaagatttggctgaattgattaataaaatgagattggctcaacaaaatgctgttacttctttgtctgaagaatgtaaaagacaaatgttgactgcttctcatactttggctgttgatgctaaaaatttgttggatgctgttgatcaagctaaagttttggctaatttggctcatccaccagctgaaggttctgctggttctgctgctggttctggtgaatttggttctgctgaagctgctgctaaagaagctgctgctaaagctggttctgctggttctgctgctggttctggtgaatttggttctggtgctatggctactccaggttctgaaaatgttttgccaagagaaccattgattgctactgctgttaaatttttgcaaaattctagagttagacaatctccattggctactagaagagcttttttgaaaaaaaaaggtttgactgatgaagaaattgatatggcttttcaacaatctggtactgctgctgatgaaccatcttctttgtgg

SEQ ID NO: 158 - DNA, 10860 bp, synthetic construct
  source 1..10860: mol_type = other DNA
atgggttctgctggttctgctgctggttctggtgaatttggttctgctggttctgctgctggttctggtgaatttggttctgctggttctgctgctggttctggtgaattttcttattatcatcatcatcatcatcatttggaatctacttctttgtataaaaaagctggttctggttctgctagaaatgcttatttgagaaaaaaaattgctagattgaaaaaagataatttgcaattggaaagagatgaacaaaatttggaaaaaattattgctaatttgagagatgaaattgctagattggaaaatgaagttgcttctcatgaacaaggttctgctggttctgctgctggttctggtgaatttgctgaagctgctgctaaagaagctgctgctaaagctggttctgctggttctgctgctggttctggtgaattttcttattatcatcatcatcatcatcatttggaatctacttctttgtataaaaaagctggttctggttctaatttggttgctcaattggaaaatgaagttgcttctttggaaaatgaaaatgaaactttgaaaaaaaaaaatttgcataaaaaagatttgattgcttatttggaaaaagaaattgctaatttgagaaaaaaaattgaagaaggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttcttcttattatcatcatcatcatcatcatttggaatctacttctttgtataaaaaagctggttctggttctcaaaaagttgctgaattgaaaaatagagttgctgttaaattgaatagaaatgaacaattgaaaaataaagttgaagaattgaaaaatagaaatgcttatttgaaaaatgaattggctactttggaaaatgaagttgctagattggaaaatgatgttgctgaaggttctgctggttctgctgctggttctggtgaatttgctgaagctgctgctaaagaagctgctgctaaagctggttctgctggttctgctgctggttctggtgaattttcttattatcatcatcatcatcatcatttggaatctacttctttgtataaaaaagctggttctggttctaatgaagttactactttggaaaatgatgctgcttttattgaaaatgaaaatgcttatttggaaaaagaaattgctagattgagaaaagaaaaagctgctttgagaaatagattggctcataaaaaaggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttctagaccaccaactatttctaatccaccaccattgatttcttctgctaaacatccatctgttggttctgctggttctgctgctggttctggtgaatttgctgaagctgctgctaaagaagctgctgctaaagctggttctgctggttctgctgctggttctggtgaatttaattttttgcaatctagaccagaaccaactgctccaccagaagaatcttttagatctggtggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttcttctaaaggtactggtttgaatccaaatgctaaagtttggcaagaaattgctccaggtaatggttctgctggttctgctgctggttctggtgaatttgctgaagctgctgctaaagaagctgctgctaaagctggttctgctggttctgctgctggttctggtgaatttccagatggtggtactacttttgaacatttgtggtcttctttggaaccagattctacttatggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttcttcttattatcatcatcatcatcatcatttggaatctacttctttgtataaaaaagctggttctggttctaaaagaattgcttatttgagaaaaaaaattgctgctttgaaaaaagataatgctaatttggaaaaagatattgctaatttggaaaatgaaattgaaagattgattaaagaaattaaaactttggaaaatgaagttgcttctcatgaacaaggttctgctggttctgctgctggttctggtgaatttgctgaagctgctgctaaagaagctgctgctaaagctggttctgctggttctgctgctggttctggtgaattttcttattatcatcatcatcatcatcatttggaatctacttctttgtataaaaaagctggttctggttctaatttgttggctactttgagatctactgctgctgttttggaaaatgaaaatcatgttttggaaaaagaaaaagaaaaattgagaaaagaaaaagaacaattgttgaataaattggaagcttataaaggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttctccagctacttctcaacatccaccaccaccaccaggtcatagatctcaagctccatctcatggttctgctggttctgctgctggttctggtgaatttgctgaagctgctgctaaagaagctgctgctaaagctggttctgctggttctgctgctggttctggtgaatttgaattgaattctttgttgattttgttggaagctgctgaatatttggaaagaagagatagaggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttctagaccaccaactatttctaatccaccaccattgatttcttctgctaaacatccatctgttggttctgctggttctgctgctggttctggtgaatttgctgaagctgctgctaaagaagctgctgctaaagctggttctgctggttctgctgctggttctggtgaatttaattttttgcaatctagaccagaaccaactgctccaccagaagaatcttttagatctggtggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttcttctaaaggtactggtttgaatccaaatgctaaagtttggcaagaaattgctccaggtaatggttctgctggttctgctgctggttctggtgaatttgctgaagctgctgctaaagaagctgctgctaaagctggttctgctggttctgctgctggttctggtgaatttccagatggtggtactacttttgaacatttgtggtcttctttggaaccagattctacttatggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttcttcttattatcatcatcatcatcatcatttggaatctacttctttgtataaaaaagctggttctggttctaaaagaattgcttatttgagaaaaaaaattgctgctttgaaaaaagataatgctaatttggaaaaagatattgctaatttggaaaatgaaattgaaagattgattaaagaaattaaaactttggaaaatgaagttgcttctcatgaacaaggttctgctggttctgctgctggttctggtgaatttgctgaagctgctgctaaagaagctgctgctaaagctggttctgctggttctgctgctggttctggtgaattttcttattatcatcatcatcatcatcatttggaatctacttctttgtataaaaaagctggttctggttctaatttgttggctactttgagatctactgctgctgttttggaaaatgaaaatcatgttttggaaaaagaaaaagaaaaattgagaaaagaaaaagaacaattgttgaataaattggaagcttataaaggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttctgctttggttgatgatgctgctgattatgaaccaccaccatctaataatgaagaagctttgggttctgctggttctgctgctggttctggtgaatttgctgaagctgctgctaaagaagctgctgctaaagctggttctgctggttctgctgctggttctggtgaatttagagaattgtttgatgatccatcttatgttaatgttcaaaatttggataaagctagacaaggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttctaaaaatactaaatctatgaattttgataatccagtttatagaaaaactactgaagaagaaggttctgctggttctgctgctggttctggtgaatttgctgaagctgctgctaaagaagctgctgctaaagctggttctgctggttctgctgctggttctggtgaatttagatctttgccatctacttggattgaaaataaattgtatggtatgtctgatccaaattggggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttctgttgttgataattctccaccaccagctttgccaccaaaaaaaagacaatctgctccatctggttctgctggttctgctgctggttctggtgaatttgctgaagctgctgctaaagaagctgctgctaaagctggttctgctggttctgctgctggttctggtgaatttactcaaagatctaaaccacaaccagctgttccaccaagaccatctgctgatttgattttgggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttctactgatgaagaaagagaagaaactgaagaagaagtttatttgttgaattctactactttgggttctgctggttctgctgctggttctggtgaatttgctgaagctgctgctaaagaagctgctgctaaagctggttctgctggttctgctgctggttctggtgaatttgatggtaatgtttctggtactcaaagattggattctgctactgttagaacttattcttgtggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttcttcttattatcatcatcatcatcatcatttggaatctacttctttgtataaaaaagctggttctggttctcaaaaagttgctcaattgaaaaatagagttgcttataaattgaaagaaaatgctaaattggaaaatattgttgctagattggaaaatgataatgctaatttggaaaaagatattgctaatttggaaaaagatattgctaatttggaaagagatgttgctagaggttctgctggttctgctgctggttctggtgaatttgctgaagctgctgctaaagaagctgctgctaaagctggttctgctggttctgctgctggttctggtgaattttcttattatcatcatcatcatcatcatttggaatctacttctttgtataaaaaagctggttctggttctaatactgttaaagaattgaaaaattatattcaagaattggaagaaagaaatgctgaattgaaaaatttgaaagaacatttgaaatttgctaaagctgaattggaatttgaattggctgctcataaatttgaaggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttctcatgatgattctttgccacatccacaacaagctactgatgattctggtcatgaatctgatggttctgctggttctgctgctggttctggtgaatttgctgaagctgctgctaaagaagctgctgctaaagctggttctgctggttctgctgctggttctggtgaatttggttctccaaatgctggttctgttgaacaaactccaaaaaaaccaggtttgagaagaagaggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttcttcttattatcatcatcatcatcatcatttggaatctacttctttgtataaaaaagctggttctggttcttttgaaaatgttactcatgaatttattttggctactttggaaaatgaaaatgctaaattgagaagattggaagctaaattggaaagagaattggctagattgagaaatgaagttgcttggttgggttctgctggttctgctgctggttctggtgaatttgctgaagctgctgctaaagaagctgctgctaaagctggttctgctggttctgctgctggttctggtgaattttcttattatcatcatcatcatcatcatttggaatctacttctttgtataaaaaagctggttctggttctcaaaaagttgaagaattgaaaaataaaattgctgaattggaaaatagaaatgctgttaaaaaaaatagagttgctcatttgaaacaagaaattgcttatttgaaagatgaattggctgctcatgaatttgaaggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttctgtttcttctactaaattggtttcttttcatgatgattctgatgaagatttgttgcatattggttctgctggttctgctgctggttctggtgaatttgctgaagctgctgctaaagaagctgctgctaaagctggttctgctggttctgctgctggttctggtgaatttgctgctgctactccaatttctacttttcatgatgattctgatgaagatttgttgcatgttggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttcttcttattatcatcatcatcatcatcatttggaatctacttctttgtataaaaaagctggttctggttctcaaaaagttgaatctttgaaacaaaaaattgaagaattgaaacaaagaaaagctcaattgaaaaatgatattgctaatttggaaaaagaaattgcttatgctgaaactggttctgctggttctgctgctggttctggtgaatttgctgaagctgctgctaaagaagctgctgctaaagctggttctgctggttctgctgctggttctggtgaattttcttattatcatcatcatcatcatcatttggaatctacttctttgtataaaaaagctggttctgaattttttagaagagaaagaaataaaatggctgctgctaaatgtagaaatagaagaagagaattgactgatactttgcaagctgaaactgatcaattggaagatgaaaaatctgctttgcaaactgaaattgctaatttgttgaaagaaaaagaaaaattggaatttattttggctgctcatagaccagcttgtaaaattccagatgatttgggttttccagaagaaatgtctttggaaggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttcttttcaaatgccagctgatactccaccaccagcttatttgccaccagaagatccaatgactggttctgctggttctgctgctggttctggtgaatttgctgaagctgctgctaaagaagctgctgctaaagctggttctgctggttctgctgctggttctggtgaatttgaaagagaatctaatgaagaaccaccaccaccatatgaagatccatattggggtaatggtggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttcttcttattatcatcatcatcatcatcatttggaatctacttctttgtataaaaaagctggttctggttctcaaaaagttgctgaattgaaaaatagagttgctgttaaattgaatagaaatgaacaattgaaaaataaagttgaagaattgaaaaatagaaatgcttatttgaaaaatgaattggctactttggaaaatgaagttgctagattggaaaatgatgttgctgaaggttctgctggttctgctgctggttctggtgaatttgctgaagctgctgctaaagaagctgctgctaaagctggttctgctggttctgctgctggttctggtgaattttcttattatcatcatcatcatcatcatttggaatctacttctttgtataaaaaagctggttctggttctaatgaagttactactttggaaaatgatgctgcttttattgaaaatgaaaatgcttatttggaaaaagaaattgctagattgagaaaagaaaaagctgctttgagaaatagattggctcataaaaaatcttattatcatcatcatcatcatcatttggaatctacttctttgtataaaaaagctggttctggttctgctagaaatgcttatttgagaaaaaaaattgctagattgaaaaaagataatttgcaattggaaagagatgaacaaaatttggaaaaaattattgctaatttgagagatgaaattgctagattggaaaatgaagttgcttctcatgaacaaggttctgctggttctgctgctggttctggtgaatttgctgaagctgctgctaaagaagctgctgctaaagctggttctgctggttctgctgctggttctggtgaattttcttattatcatcatcatcatcatcatttggaatctacttctttgtataaaaaagctggttctggttctaatttggttgctcaattggaaaatgaagttgcttctttggaaaatgaaaatgaaactttgaaaaaaaaaaatttgcataaaaaagatttgattgcttatttggaaaaagaaattgctaatttgagaaaaaaaattgaagaaggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttctgaacaaaaattgatttctgaagaagatttggaacaaaaattgatttctgaagaagatttggaacaaaaattgatttctgaagaagatttgggttctgctggttctgctgctggttctggtgaatttggttctgctggttctgctgctggttctggtgaatttggttctgctggttctgctgctggttctggtgaattt

SEQ ID NO: 159 - DNA, 1356 bp, synthetic construct
  source 1..1356: mol_type = other DNA
atgggttctgctggttctgctgctggttctggtgaatttggttctgctggttctgctgctggttctggtgaatttggttctgctggttctgctgctggttctggtgaattttcttattatcatcatcatcatcatcatttggaatctacttctttgtataaaaaagctggttctggttctgctagaaatgcttatttgagaaaaaaaattgctagattgaaaaaagataatttgcaattggaaagagatgaacaaaatttggaaaaaattattgctaatttgagagatgaaattgctagattggaaaatgaagttgcttctcatgaacaaggttctgctggttctgctgctggttctggtgaatttgctgaagctgctgctaaagaagctgctgctaaagctggttctgctggttctgctgctggttctggtgaattttcttattatcatcatcatcatcatcatttggaatctacttctttgtataaaaaagctggttctggttctaatttggttgctcaattggaaaatgaagttgcttctttggaaaatgaaaatgaaactttgaaaaaaaaaaatttgcataaaaaagatttgattgcttatttggaaaaagaaattgctaatttgagaaaaaaaattgaagaaggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttcttctgctactagagaattggatgaattgatggcttctttgtctgattttaaaattcaaggtggttctgctggttctgctgctggttctggtgaatttgctgaagctgctgctaaagaagctgctgctaaagctggttctgctggttctgctgctggttctggtgaatttgatttggctttgtctgaaaattgggctcaagaatttttggctgctggtgatgctgttgatggttctgctggttctgctgctggttctggtgaatttggttctgctgaagctgctgctaaagaagctgctgctaaagaagctgctgctaaagaagctgctgctaaagctggttctgctggttctgctgctggttctggtgaatttggttctgattataaagatgatgatgataaagattataaagatgatgatgataaagattataaagatgatgatgataaaggttctgctggttctgctgctggttctggtgaatttggttctgctggttctgctgctggttctggtgaatttggttctgctggttctgctgctggttctggtgaattt

SEQ ID NO: 160 - protein, 1532 aa, synthetic construct
MSAKAISEQTGKELLYKFICTTSAIQNRFKYARVTPDTDWARLLQDHPWLLSQNLVVKPDQLIKRRGKLGLVGVNLTLDGVKSWLKPRLGQEATVGKATGFLKNFLIEPFVPHSQAEEFYVCIYATREGDYVLFHHEGGVDVGDVDAKAQKLLVGVDEKLNPEDIKKHLLVHAPEDKKEILASFISGLFNFYEDLYFTYLEINPLVVTKDGVYVLDLAAKVDATADYICKVKWGDIEFPPPFGREAYPEEAYIADLDAKSGASLKLTLLNPKGRIWTMVAGGGASVVYSDTICDLGGVNELANYGEYSGAPSEQQTYDYAKTILSLMTREKHPDGKILIIGGSIANFTNVAATFKGIVRAIRDYQGPLKEHEVTIFVRRGGPNYQEGLRVMGEVGKTTGIPIHVFGTETHMTAIVGMALGHRPIPNQPPTAAHTANFLLNASGSTSTPAPSRTASFSESRADEVAPAKKAKPAMPQDSVPSPRSLQGKSTTLFSRHTKAIVWGMQTRAVQGMLDFDYVCSRDEPSVAAMVYPFTGDHKQKFYWGHKEILIPVFKNMADAMRKHPEVDVLINFASLRSAYDSTMETMNYAQIRTIAIIAEGIPEALTRKLIKKADQKGVTIIGPATVGGIKPGCFKIGNTGGMLDNILASKLYRPGSVAYVSRSGGMSNELNNIISRTTDGVYEGVAIGGDRYPGSTFMDHVLRYQDTPGVKMIVVLGEIGGTEEYKICRGIKEGRLTKPIVCWCIGTCATMFSSEVQFGHAGACANQASETAVAKNQALKEAGVFVPRSFDELGEIIQSVYEDLVANGVIVPAQEVPPPTVPMDYSWARELGLIRKPASFMTSICDERGQELIYAGMPITEVFKEEMGIGGVLGLLWFQKRLPKYSCQFIEMCLMVTADHGPAVSGAHNTIICARAGKDLVSSLTSGLLTIGDRFGGALDAAAKMFSKAFDSGIIPMEFVNKMKKEGKLIMGIGHRVKSINNPDMRVQILKDYVRQHFPATPLLDYALEVEKITTSKKPNLILNVDGLIGVAFVDMLRNCGSFTREEADEYIDIGALNGIFVLGRSMGFIGHYLDQKRLKQGLYRHPWDDISYVLPEHMSMKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASSYYHHHHHHLESTSLYKKAGSGSNLVAQLENEVASLENENETLKKKNLHKKDLIAYLEKEIANLRKKIEEGSAGSAAGSGEFGSAEAAAKEAAAKAGSAGSAAGSGEFGSSYYHHHHHHLESTSLYKKAGSGSARNAYLRKKIARLKKDNLQLERDEQNLEKIIANLRDEIARLENEVASHEQGSG

SEQ ID NO: 161 - protein, 824 aa, synthetic construct
MKNCVIVSAVRTAIGSFNGSLASTSAIDLGATVIKAAIERAKIDSQHVDEVIMGNVLQAGLGQNPARQALLKSGLAETVCGFTVNKVCGSGLKSVALAAQAIQAGQAQSIVAGGMENMSLAPYLLDAKARSGYRLGDGQVYDVILRDGLMCATHGYHMGITAENVAKEYGITREMQDELALHSQRKAAAAIESGAFTAEIVPVNVVTRKKTFVFSQDEFPKANSTAEALGALRPAFDKAGTVTAGNASGINDGAAALVIMEESAALAAGLTPLARIKSYASGGVPPALMGMGPVPATQKALQLAGLQLADIDLIEANEAFAAQFLAVGKNLGFDSEKVNVNGGAIALGHPIGASGARILVTLLHAMQARDKTLGLATLCIGGGQGIAMVIERLNKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASSYYHHHHHHLESTSLYKKAGSGSNEVTTLENDAAFIENENAYLEKEIARLRKEKAALRNRLAHKKGSAGSAAGSGEFGSAEAAAKEAAAKAGSAGSAAGSGEFGSSYYHHHHHHLESTSLYKKAGSGSQKVAELKNRVAVKLNRNEQLKNKVEELKNRNAYLKNELATLENEVARLENDVAEGSG

SEQ ID NO: 162 - protein, 779 aa, synthetic construct
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKRPDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSKKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASENLYFQGENLYFQGDSSESCWNCGRKASETCSGCNTARYCGSFCQHKDWEKHHHICGQTLQAQQGSAGSAAGSGEFGSAEAAAKEAAAKAGSAGSAAGSGEFGSMAVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRPGSG

SEQ ID NO: 163 - protein, 744 aa, synthetic construct
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNRKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASGPLGSPLTASMLASAPPQEQKQMLGERLFPLIQAMHPTLAGKITGMLLEIDNSELLHMLESPESLRSKVDEAVAVLQAHQAKEAAQKAGSAGSAAGSGEFGSAEAAAKEAAAKAGSAGSAAGSGEFGSNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVGSG

SEQ ID NO: 164 - protein, 823 aa, synthetic construct
MIVKPMVRNNICLNAHPQGCKKGVEDQIEYTKKRITAEVKAGAKAPKNVLVLGCSNGYGLASRITAAFGYGAATIGVSFEKAGSETKYGTPGWYNNLAFDEAAKREGLYSVTIDGDAFSDEIKAQVIEEAKKKGIKFDLIVYSLASPVRTDPDTGIMHKSVLKPFGKTFTGKTVDPFTGELKEISAEPANDEEAAATVKVMGGEDWERWIKQLSKEGLLEEGCITLAYSYIGPEATQALYRKGTIGKAKEHLEATAHRLNKENPSIRAFVSVNKGLVTRASAVIPVIPLYLASLFKVMKEKGNHEGCIEQITRLYAERLYRKDGTIPVDEENRIRIDDWELEEDVQKAVSALMEKVTGENAESLTDLAGYRHDFLASNGFDVEGINYEAEVERFDRIKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASSYYHHHHHHLESTSLYKKAGSGSNLLATLRSTAAVLENENHVLEKEKEKLRKEKEQLLNKLEAYKGSAGSAAGSGEFGSAEAAAKEAAAKAGSAGSAAGSGEFGSSYYHHHHHHLESTSLYKKAGSGSKRIAYLRKKIAALKKDNANLEKDIANLENEIERLIKEIKTLENEVASHEQGSG

SEQ ID NO: 165 - protein, 829 aa, synthetic construct
MTREVVVVSGVRTAIGTFGGSLKDVAPAELGALVVREALARAQVSGDDVGHVVFGNVIQTEPRDMYLGRVAAVNGGVTINAPALTVNRLCGSGLQAIVSAAQTILLGDTDVAIGGGAESMSRAPYLAPAARWGARMGDAGLVDMMLGALHDPFHRIHMGVTAENVAKEYDISRAQQDEAALESHRRASAAIKAGYFKDQIVPVVSKGRKGDVTFDTDEHVRHDATIDDMTKLRPVFVKENGTVTAGNASGLNDAAAAVVMMERAEAERRGLKPLARLVSYGHAGVDPKAMGIGPVPATKIALERAGLQVSDLDVIEANEAFAAQACAVTKALGLDPAKVNPNGSGISLGHPIGATGALITVKALHELNRVQGRYALVTMCIGGGQGIAAIFERIKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASDVMWEYKWENTGDAELYGPFTSAQMQTWVSEGYFPDGVYCRKLDPPGGQFYNSKRIDFDLYTGSAGSAAGSGEFGSAEAAAKEAAAKAGSAGSAAGSGEFGSESDSVEFNNAISYVNKIKTRFLDHPEIYRSFLEILHTYQKEQLHTKGRPFRGMSEEEVFTEVANLFRGQEDLLSEFGQFLPEAKRGSG

SEQ ID NO: 166 - protein, 852 aa, synthetic construct
MKLSTKLCWCGIKGRLRPQKQQQLHNTNLQMTELKKQKTAEQKTRPQNVGIKGIQIYIPTQCVNQSELEKFDGVSQGKYTIGLGQTNMSFVNDREDIYSMSLTVLSKLIKSYNIDTNKIGRLEVGTETLIDKSKSVKSVLMQLFGENTDVEGIDTLNACYGGTNALFNSLNWIESNAWDGRDAIVVCGDIAIYDKGAARPTGGAGTVAMWIGPDAPIVFDSVRASYMEHAYDFYKPDFTSEYPYVDGHFSLTCYVKALDQVYKSYSKKAISKGLVSDPAGSDALNVLKYFDYNVFHVPTCKLVTKSYGRLLYNDFRANPQLFPEVDAELATRDYDESLTDKNIEKTFVNVAKPFHKERVAQSLIVPTNTGNMYTASVYAAFASLLNYVGSDDLQGKRVGLFSYGSGLAASLYSCKIVGDVQHIIKELDITNKLAKRITETPKDYEAAIELRENAHLKKNFKPQGSIEHLQSGVYYLTNIDDKFRRSYDVKKKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASLGPLPPGWEVRSTVSGRIYFVDHNNRTTQFTDPRLHGSAGSAAGSGEFGSAEAAAKEAAAKAGSAGSAAGSGEFGSGAMGPLPPGWEKRTDSNGRVYFVNHNTRITQWEDPRSGSG

SEQ ID NO: 167 - protein, 829 aa, synthetic construct
MVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASSYYHHHHHHLESTSLYKKAGSEFFRRERNKMAAAKCRNRRRELTDTLQAETDQLEDEKSALQTEIANLLKEKEKLEFILAAHRPACKIPDDLGFPEEMSLEGSAGSAAGSGEFGSAEAAAKEAAAKAGSAGSAAGSGEFGSSYYHHHHHHLESTSLYKKAGSGSQKVESLKQKIEELKQRKAQLKNDIANLEKEIAYAETGSG

SEQ ID NO: 168 - protein, 1049 aa, synthetic construct
  REGION 1..1049: note = chimeric enzyme
MSLPFLTSAPGKVIIFGEHSAVYNKPAVAASVSALRTYLLISESSAPDTIELDFPDISFNHKWSINDFNAITEDQVNSQKLAKAQQATDGLSQELVSLLDPLLAQLSESFHYHAAFCFLYMFVCLCPHAKNIKFSLKSTLPIGAGLGSSASISVSLALAMAYLGGLIGSNDLEKLSENDKHIVNQWAFIGEKCIHGTPSGIDNAVATYGNALLFEKDSHNGTINTNNFKFLDDFPAIPMILTYTRIPRSTKDLVARVRVLVTEKFPEVMKPILDAMGECALQGLEIMTKLSKCKGTDDEAVETNNELYEQLLELIRINHGLLVSIGVSHPGLELIKNLSDDLRIGSTKLTGAGGGGCSLTLLRRDITQEQIDSFKKKLQDDFSYETFETDLGGTGCCLLSAKNLNKDLKIKSLVFQLFENKTTTKQQIDDLLLPGNTNLPWTSKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASMEPAMEPETLEARINRATNPLNKELDWASINGFCEQLNEDFEGPPLATRLLAHKIQSPQEWEAIQALTVLETCMKSCGKRFHDEVGKFRFLNELIKVVSPKYLGSRTSEKVKNKILELLYSWTVGLPEEVKIAEAYQMLKKQGIVKSGSAGSAAGSGEFGSAEAAAKEAAAKAGSAGSAAGSGEFGSGAMGSMAEAEGESLESWLNKATNPSNRQEDWEYIIGFCDQINKELEGPQIAVRLLAHKIQSPQEWEALQALTVLEACMKNCGRRFHNEVGKFRFLNELIKVVSPKYLGDRVSEKVKTKVIELLYSWTMALPEEAKIKDAYHMLKRQGIVQSDPPIPVDRTLIPSPPPRPKNGSG

SEQ ID NO: 169 - protein, 871 aa, synthetic construct
MSELRAFSAPGKALLAGGYLVLDTKYEAFVVGLSARMHAVAHPYGSLQGSDKFEVRVKSKQFKDGEWLYHISPKSGFIPVSIGGSKNPFIEKVIANVFSYFKPNMDDYCNRNLFVIDIFSDDAYHSQEDSVTEHRGNRRLSFHSHRIEEVPKTGLGSSAGGLVTVLTTALASFFVSDLENNVDKYREVIHNLAQVAHCQAQGKIGSGFDVAAAAYGSIRYRRFPPALISNLPDIGSATYGSKLAHLVDEEDWNITIKSNHLPSGLTLWMGDIKNGSETVKLVQKVKNWYDSHMPESLKIYTELDHANSRFMDGLSKLDRLHETHDDYSDQIFESLERNDCTCQKYPEITEVRDAVATIRRSFRKITKESGADIEPPVQTSLLDDCQTLKGVLTCLIPGAGGYDAIAVITKQDVDLRAQTANDKRFSKVQWLDVTQADWGVRKEKDPETYLDKKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASSYYHHHHHHLESTSLYKKAGSGSQKVEELKNKIAELENRNAVKKNRVAHLKQEIAYLKDELAAHEFEGSAGSAAGSGEFGSAEAAAKEAAAKAGSAGSAAGSGEFGSSYYHHHHHHLESTSLYKKAGSGSFENVTHEFILATLENENAKLRRLEAKLERELARLRNEVAWLGSG

SEQ ID NO: 170 - protein, 1011 aa, synthetic construct
MTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKEKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASAMADLEQKVLEMEASTYDGVFIWKISDFPRKRQEAVAGRIPAIFSPAFYTSRYGYKMCLRIYLNGDGTGRGTHLSLFFVVMKGPNDALLRWPFNQKVTLMLLDQNNREHVIDAFRPDVTSSSFQRPVNDMNIASGCPLFCPVSKMEAKNSYVRDDAIFIKAIVDLTGLGSAGSAAGSGEFGSAEAAAKEAAAKAGSAGSAAGSGEFGSASIKLQSSDGEIFEVDVEIAKQSVTIKTMLEDLGMDDEGDDDPVPLPNVNAAILKKVIQWCTHHKDDPPPPEDDENKEKRTDDIPVWDQEFLKVDQGTLFELILAANYLDIKGLLDVTCKTVANMIKGKTPEEIRKTFNIKNDFTEEEEAQVRKENQWCGSG

SEQ ID NO: 171 - protein, 720 aa, synthetic construct
MTADNNSMPHGAVSSYAKLVQNQTPEDILEEFPEIIPLQQRPNTRSSETSNDESGETCFSGHDEEQIKLMNENCIVLDWDDNAIGAGTKKVCHLMENIEKGLLHRAFSVFIFNEQGELLLQQRATEKITFPDLWTNTCCSHPLCIDDELGLKGKLDDKIKGAITAAVRKLDHELGIPEDETKTRGKFHFLNRIHYMAPSNEPWGEHEIDYILFYKINAKENLTVNPNVNEVRDFKWVSPNDLKTMFADPSYKFTPWFKIICENYLFNWWEQLDDLSEVENDRQIHRMLKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASSYYHHHHHHLESTSLYKKAGSGSNTVKELKNYIQELEERNAELKNLKEHLKFAKAELEFELAAHKFEGSAGSAAGSGEFGSAEAAAKEAAAKAGSAGSAAGSGEFGSSYYHHHHHHLESTSLYKKAGSGSQKVAQLKNRVAYKLKENAKLENIVARLENDNANLEKDIANLEKDIANLERDVARGSG

SEQ ID NO: 172 - protein, 801 aa, synthetic construct
MEAKIDELINNDPVWSSQNESLISKPYNHILLKPGKNFRLNLIVQINRVMNLPKDQLAIVSQIVELLHNSSLLIDDIEDNAPLRRGQTTSHLIWGVPSTINTANYMYFRAMQLVSQLTTKEPLYHWLITIFNEELINLHRGQGLDIYWRDFLPEIIPTQEMYLNMVMNKTGGLFRLTLRLMEALSPSSHHGHSLVPFINLLGIIYQIRDDYLNLKDFQMSSEKGFAEDITEGKLSFPIVHALNFTKTKGQTEQHNEILRILLLRTSDKDIKLKLIQILEFDTNSLAYTKNFINQLVNMIKNDNENKYLPDLASHSDTATNLHDELLYIIDHLSELKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASLCTMKKGPSGYGFNLHSDKSKPGQFIRSVDPDSPAEASGLRAQDRIVEVNGVCMEGKQHGDVVSAIRAGGDETKLLVVDREGSAGSAAGSGEFGSAEAAAKEAAAKAGSAGSAAGSGEFGSSSGAIIYTVELKRYGGPLGITISGTEEPFDPIIISSLTKGGLAERTGAIHIGDRILAINSSSLKGKPLSEAIHLLQMAGETVTLKIKKQTDAQPASSGSG

SEQ ID NO: 173 - protein, 547 aa, synthetic construct
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRHGSG

SEQ ID NO: 174 - protein, 548 aa, synthetic construct
MNCSTFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAACKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHHGSG

SEQ ID NO: 175 - protein, 886 aa, synthetic construct
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKYKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASGNNLETYEWYNKSISRDKAEKLLLDTGKEGAFMVRDSRTPGTYTVSVFTKAIISENPCIKHYHIKETNDSPKRYYVAEKYVFDSIPLLIQYHQYNGGGLVTRLRYPVCGGSAGSAAGSGEFGSAEAAAKEAAAKAGSAGSAAGSGEFGSGSHPWFFGKIPRAKAEEMLSKQRHDGAFLIRESESAPGDFSLSVKFGNDVQHFKVLRDGAGKYFLWVVKFNSLNELVDYHRSTSVSRNQQIFLRDIEQVPQQPTGSG

SEQ ID NO: 176 - protein, 668 aa, synthetic construct
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRKKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASGQDRSEATLIKRFKGEGVRYKAKLIGIDEVSAARGDKLCQDSMMKLKGVVAGARSKGEHKQKIFLTISFGGIKIFDEKTGALQHHHAVHEISYIAKDITDHRAFGYVCGKEGNHRFVAIKTAQAAEPVILDLRDLFQLIYELKQREELEKKAGSAGSAAGSGEFGSAEAAAKEAAAKAGSAGSAAGSGEFGSGSHMGSQFWVTSQKTEASERCGLQGSYILRVEAEKLTLLTLGAQSQILEPLLFWPYTLLRRYGRDKVMFSFEAGRRCPSGPGTFTFQTSQGNDIFQAVEAAIQQQKAQGKVGQAQDILRLEHHHHHHGSG

SEQ ID NO: 177 - protein, 800 aa, synthetic construct
MGLSSVCTFSFQTNYHTLLNPHNNNPKTSLLCYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFIKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASAEYVRALFDFNGNDEEDLPFKKGDILRIRDKPEEQWWNAEDSEGKRGMIPVPYVEKYGSAGSAAGSGEFGSAEAAAKEAAAKAGSAGSAAGSGEFGSLIKHMRAEALFDFTGNSKLELNFKAGDVIFLLSRINKDWLEGTVRGATGIFPLSFVKILKGSG

SEQ ID NO: 178 - protein, 2744 aa, synthetic construct
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLSDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVSVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLKKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASGSHMRLGAQSIQPTANLDRTDDLVYLNVMELVRAVLELKNELAQLPPEGYVVVVKNVGLTLRKLIGSVDDLLPSLPSSSRTEIEGTQKLLNKDLAELINKMRLAQQNAVTSLSEECKRQMLTASHTLAVDAKNLLDAVDQAKVLANLAHPPAEGSAGSAAGSGEFGSAEAAAKEAAAKAGSAGSAAGSGEFGSGAMATPGSENVLPREPLIATAVKFLQNSRVRQSPLATRRAFLKKKGLTDEEIDMAFQQSGTAADEPSSLWGSG

SEQ ID NO: 179 - protein, 3623 aa, synthetic construct
MGSAGSAAGSGEFGSAGSAAGSGEFGSAGSAAGSGEFSYYHHHHHHLESTSLYKKAGSGSARNAYLRKKIARLKKDNLQLERDEQNLEKIIANLRDEIARLENEVASHEQGSAGSAAGSGEFAEAAAKEAAAKAGSAGSAAGSGEFSYYHHHHHHLESTSLYKKAGSGSNLVAQLENEVASLENENETLKKKNLHKKDLIAYLEKEIANLRKKIEEGSAGSAAGSGEFGSAEAAAKEAAAKEAAAKEAAAKAGSAGSAAGSGEFGSSYYHHHHHHLESTSLYKKAGSGSQKVAELKNRVAVKLNRNEQLKNKVEELKNRNAYLKNELATLENEVARLENDVAEGSAGSAAGSGEFAEAAAKEAAAKAGSAGSAAGSGEFSYYHHHHHHLESTSLYKKAGSGSNEVTTLENDAAFIENENAYLEKEIARLRKEKAALRNRLAHKKGSAGSAAGSGEFGSAEAAAKEAAAKEAAAKEAAAKAGSAGSAAGSGEFGSRPPTISNPPPLISSAKHPSVGSAGSAAGSGEFAEAAAKEAAAKAGSAGSAAGSGEFNFLQSRPEPTAPPEESFRSGGSAGSAAGSGEFGSAEAAAKEAAAKEAAAKEAAAKAGSAGSAAGSGEFGSSKGTGLNPNAKVWQEIAPGNGSAGSAAGSGEFAEAAAKEAAAKAGSAGSAAGSGEFPDGGTTFEHLWSSLEPDSTYGSAGSAAGSGEFGSAEAAAKEAAAKEAAAKEAAAKAGSAGSAAGSGEFGSSYYHHHHHHLESTSLYKKAGSGSKRIAYLRKKIAALKKDNANLEKDIANLENEIERLIKEIKTLENEVASHEQGSAGSAAGSGEFAEAAAKEAAAKAGSAGSAAGSGEFSYYHHHHHHLESTSLYKKAGSGSNLLATLRSTAAVLENENHVLEKEKEKLRKEKEQLLNKLEAYKGSAGSAAGSGEFGSAEAAAKEAAAKEAAAKEAAAKAGSAGSAAGSGEFGSPATSQHPPPPPGHRSQAPSHGSAGSAAGSGEFAEAAAKEAAAKAGSAGSAAGSGEFELNSLLILLEAAEYLERRDRGSAGSAAGSGEFGSAEAAAKEAAAKEAAAKEAAAKAGSAGSAAGSGEFGSRPPTISNPPPLISSAKHPSVGSAGSAAGSGEFAEAAAKEAAAKAGSAGSAAGSGEFNFLQSRPEPTAPPEESFRSGGSAGSAAGSGEFGSAEAAAKEAAAKEAAAKEAAAKAGSAGSAAGSGEFGSSKGTGLNPNAKVWQEIAPGNGSAGSAAGSGEFAEAAAKEAAAKAGSAGSAAGSGEFPDGGTTFEHLWSSLEPDSTYGSAGSAAGSGEFGSAEAAAKEAAAKEAAAKEAAAKAGSAGSAAGSGEFGSSYYHHHHHHLESTSLYKKAGSGSKRIAYLRKKIAALKKDNANLEKDIANLENEIERLIKEIKTLENEVASHEQGSAGSAAGSGEFAEAAAKEAAAKAGSAGSAAGSGEFSYYHHHHHHLESTSLYKKAGSGSNLLATLRSTAAVLENENHVLEKEKEKLRKEKEQLLNKLEAYKGSAGSAAGSGEFGSAEAAAKEAAAKEAAAKEAAAKAGSAGSAAGSGEFGSALVDDAADYEPPPSNNEEALGSAGSAAGSGEFAEAAAKEAAAKAGSAGSAAGSGEFRELFDDPSYVNVQNLDKARQGSAGSAAGSGEFGSAEAAAKEAAAKEAAAKEAAAKAGSAGSAAGSGEFGSKNTKSMNFDNPVYRKTTEEEGSAGSAAGSGEFAEAAAKEAAAKAGSAGSAAGSGEFRSLPSTWIENKLYGMSDPNWGSAGSAAGSGEFGSAEAAAKEAAAKEAAAKEAAAKAGSAGSAAGSGEFGSVVDNSPPPALPPKKRQSAPSGSAGSAAGSGEFAEAAAKEAAAKAGSAGSAAGSGEFTQRSKPQPAVPPRPSADLILGSAGSAAGSGEFGSAEAAAKEAAAKEAAAKEAAAKAGSAGSAAGSGEFGSTDEEREETEEEVYLLNSTTLGSAGSAAGSGEFAEAAAKEAAAKAGSAGSAAGSGEFDGNVSGTQRLDSATVRTYSCGSAGSAAGSGEFGSAEAAAKEAAAKEAAAKEAAAKAGSAGSAAGSGEFGSSYYHHHHHHLESTSLYKKAGSGSQKVAQLKNRVAYKLKENAKLENIVARLENDNANLEKDIANLEKDIANLERDVARGSAGSAAGSGEFAEAAAKEAAAKAGSAGSAAGSGEFSYYHHHHHHLESTSLYKKAGSGSNTVKELKNYIQELEERNAELKNLKEHLKFAKAELEFELAAHKFEGSAGSAAGSGEFGSAEAAAKEAAAKEAAAKEAAAKAGSAGSAAGSGEFGSHDDSLPHPQQATDDSGHESDGSAGSAAGSGEFAEAAAKEAAAKAGSAGSAAGSGEFGSPNAGSVEQTPKKPGLRRRGSAGSAAGSGEFGSAEAAAKEAAAKEAAAKEAAAKAGSAGSAAGSGEFGSSYYHHHHHHLESTSLYKKAGSGSFENVTHEFILATLENENAKLRRLEAKLERELARLRNEVAWLGSAGSAAGSGEFAEAAAKEAAAKAGSAGSAAGSGEFSYYHHHHHHLESTSLYKKAGSGSQKVEELKNKIAELENRNAVKKNRVAHLKQEIAYLKDELAAHEFEGSAGSAAGSGEFGSAEAAAKEAAAKEAAAKEAAAKAGSAGSAAGSGEFGSVSSTKLVSFHDDSDEDLLHIGSAGSAAGSGEFAEAAAKEAAAKAGSAGSAAGSGEFAAATPISTFHDDSDEDLLHVGSAGSAAGSGEFGSAEAAAKEAAAKEAAAKEAAAKAGSAGSAAGSGEFGSSYYHHHHHHLESTSLYKKAGSGSQKVESLKQKIEELKQRKAQLKNDIANLEKEIAYAETGSAGSAAGSGEFAEAAAKEAAAKAGSAGSAAGSGEFSYYHHHHHHLESTSLYKKAGSEFFRRERNKMAAAKCRNRRRELTDTLQAETDQLEDEKSALQTEIANLLKEKEKLEFILAAHRPACKIPDDLGFPEEMSLEGSAGSAAGSGEFGSAEAAAKEAAAKEAAAKEAAAKAGSAGSAAGSGEFGSFQMPADTPPPAYLPPEDPMTGSAGSAAGSGEFAEAAAKEAAAKAGSAGSAAGSGEFERESNEEPPPPYEDPYWGNGGSAGSAAGSGEFGSAEAAAKEAAAKEAAAKEAAAKAGSAGSAAGSGEFGSSYYHHHHHHLESTSLYKKAGSGSQKVAELKNRVAVKLNRNEQLKNKVEELKNRNAYLKNELATLENEVARLENDVAEGSAGSAAGSGEFAEAAAKEAAAKAGSAGSAAGSGEFSYYHHHHHHLESTSLYKKAGSGSNEVTTLENDAAFIENENAYLEKEIARLRKEKAALRNRLAHKKSYYHHHHHHLESTSLYKKAGSGSARNAYLRKKIARLKKDNLQLERDEQNLEKIIANLRDEIARLENEVASHEQGSAGSAAGSGEFAEAAAKEAAAKAGSAGSAAGSGEFSYYHHHHHHLESTSLYKKAGSGSNLVAQLENEVASLENENETLKKKNLHKKDLIAYLEKEIANLRKKIEEGSAGSAAGSGEFGSAEAAAKEAAAKEAAAKEAAAKAGSAGSAAGSGEFGSEQKLISEEDLEQKLISEEDLEQKLISEEDLGSAGSAAGSGEFGSAGSAAGSGEFGSAGSAAGSGEFGSG

SEQ ID NO: 180 - protein, 455 aa, synthetic construct
MGSAGSAAGSGEFGSAGSAAGSGEFGSAGSAAGSGEFSYYHHHHHHLESTSLYKKAGSGSARNAYLRKKIARLKKDNLQLERDEQNLEKIIANLRDEIARLENEVASHEQGSAGSAAGSGEFAEAAAKEAAAKAGSAGSAAGSGEFSYYHHHHHHLESTSLYKKAGSGSNLVAQLENEVASLENENETLKKKNLHKKDLIAYLEKEIANLRKKIEEGSAGSAAGSGEFGSAEAAAKEAAAKEAAAKEAAAKAGSAGSAAGSGEFGSSATRELDELMASLSDFKIQGGSAGSAAGSGEFAEAAAKEAAAKAGSAGSAAGSGEFDLALSENWAQEFLAAGDAVDGSAGSAAGSGEFGSAEAAAKEAAAKEAAAKEAAAKAGSAGSAAGSGEFGSDYKDDDDKDYKDDDDKDYKDDDDKGSAGSAAGSGEFGSAGSAAGSGEFGSAGSAAGSGEFGSG

SEQ ID NO: 181 - DNA, 4596 bp, synthetic construct
  source 1..4596: mol_type = other DNA
atgagtgctaaggcaatttctgaacaaactggtaaagaattgttgtacaagtttatttgtactacatcagccatccaaaatagattcaaatacgctagagttaccccagatactgactgggctagattgttacaagatcatccatggttgttatctcaaaacttggttgtcaaacctgaccaattaattaagagaagaggtaaattgggtttagtaggtgttaatttgacattggatggtgtaaagtcttggttgaaaccaagattaggtcaagaagccacagttggtaaagctaccggtttcttgaaaaatttcttgatcgaaccatttgtccctcattcacaagccgaagaattctatgtatgtatctacgctactagagagggtgactatgttttatttcatcacgaaggtggtgtcgacgtaggtgacgttgacgccaaggctcaaaagttgttggttggtgtcgatgaaaagttgaacccagaagacattaaaaagcatttgttggttcacgcacctgaagataaaaaggaaatattggcctcctttataagtggtttgtttaatttctacgaagatttgtacttcacctacttggaaattaacccattagtagttactaaggatggtgtatatgttttggacttagctgcaaaagttgatgcaacagccgactacatttgtaaggtcaaatggggtgacatcgaatttccacctccattcggtagagaagcttatccagaagaagcctacattgctgatttggacgctaagtctggtgcatcattgaagttgacattgttgaaccctaaaggtagaatttggaccatggttgctggtggtggtgctagtgtcgtatattctgatactatatgcgacttgggtggtgttaacgaattggcaaactacggtgaatactcaggtgccccatccgaacaacaaacatacgattacgctaagaccatcttgtccttaatgactagagaaaagcatcctgatggtaaaatcttgatcatcggtggtagtatcgcaaattttactaacgttgccgctacattcaagggtatcgtcagagctataagagattaccaaggtccattgaaggaacacgaagtaacaatattcgttagaagaggtggtcctaactaccaagaaggtttgagagtcatgggtgaagtaggtaaaaccactggtataccaatccatgtctttggtacagaaacccacatgactgcaatagttggtatggccttaggtcatagaccaatccctaatcaacctccaaccgcagcccacactgcaaatttcttgttaaacgcctctggttcaacttccacaccagctccttctagaacagcaagtttctctgaatcaagagctgatgaagtcgctccagctaagaaagcaaaaccagccatgcctcaagactccgttccaagtcctagatctttgcagggtaaatctactactttgttttctagacatactaaggctatagtatggggtatgcaaacaagagcagttcaaggcatgttggatttcgactatgtttgtagtagagatgaaccatctgttgctgcaatggtctatccttttactggtgaccataagcaaaaattctactggggtcacaaggaaatattgatcccagtttttaagaacatggccgatgctatgagaaaacatcctgaagtcgacgtattgattaacttcgcctcattaagatccgcttacgattctacaatggaaaccatgaactacgctcaaataagaaccatcgctatcattgcagaaggtattccagaagccttgactagaaagttgattaagaaagctgatcaaaaaggtgtcacaataatcggtccagctaccgtaggtggtattaagcctggttgtttcaagatcggtaacactggtggtatgttggataacatattggcatctaagttgtatagaccaggttcagtcgcttacgtatccagaagtggtggtatgtccaacgaattgaacaacatcatcagtagaactacagatggtgtatacgaaggtgttgctattggtggtgacagatacccaggttctacttttatggatcatgtattgagatatcaagacacacctggtgttaaaatgattgttgtcttgggtgaaataggtggtactgaagaatacaagatatgcagaggtatcaaagaaggtagattgacaaagccaatcgtttgttggtgcattggtacttgtgcaacaatgttttcttcagaagttcaattcggtcatgcaggtgcctgcgctaatcaagcttcagaaacagcagttgccaagaaccaagcattaaaagaagccggtgtttttgtccctagatctttcgatgaattaggtgaaatcattcaatcagtctatgaagacttggtagctaatggtgtaattgttccagcacaagaagttcctccacctactgtccctatggattactcttgggcaagagaattgggtttaattagaaagccagctagttttatgacctctatatgtgatgaaagaggtcaagaattgatctatgctggtatgcctattactgaagtattcaaagaagaaatgggtatcggtggtgttttaggtttgttgtggttccaaaagagattgccaaagtactcttgtcaattcattgaaatgtgcttaatggttacagctgatcatggtcctgctgtctcaggtgcacacaataccataatctgcgctagagctggtaaagatttggtttcttctttgacctcaggtttgttaactattggtgacagatttggtggtgcattagacgccgctgcaaagatgttttcaaaagctttcgattccggtataatcccaatggaattcgttaataagatgaaaaaggagggtaaattgataatgggtatcggtcatcgtgttaagtctatcaataaccctgatatgagagtacaaatcttgaaggactatgttagacaacactttccagccacacctttgttagattacgctttggaagttgaaaagattaccacttctaaaaagccaaatttgatcttgaacgttgatggtttaattggtgttgcttttgtcgacatgttgagaaactgtggttccttcactagagaagaagctgatgaatatatcgacattggtgcattgaatggtatctttgttttaggtagatctatgggtttcattggtcattacttggatcaaaagagattaaagcaaggtttgtacagacatccatgggatgacatttcttacgttttacctgaacacatgtcaatgaaattgtctggtggtggtggttctggtggtggtggttctggtggtggtggtagtgccgaagcttggtacaatttgggtaacgcatactacaagcagggtgactaccaaaaggcaattgaatattaccaaaaggccttggaattagacccaaataacgcagaagcctggtataatttgggtaatgcttattataaacagggtgactatcaaaaggctatcgaatactaccaaaaggcattggaattagaccctaataacgctgaagcatggtataatttgggtaacgcttattataagcagggtgactatcaaaaagccatcgaagactaccaaaaggctttggaattagatccaaataacttacaagccgaagcttggaagaatttgggtaacgcttactataaacagggtgactaccaaaaagcaattgaatactatcaaaaagctttagaattggaccctaataacgcatcagcctggtacaatttgggtaatgcttactataagcagggtgactatcagaaggccattgaatactatcaaaaggctttagaattggatccaaataacgctaaagcatggtacagacgtggtaacgcttattacaaacagggtgactaccagaaagccattgaagattatcaaaaggctttggaattggatcctaacaacagatctagatcagctggtggtggtggttctggtggtggtggttctggtggtggtggtgcttcttcatattaccatcaccatcaccatcacttggaatccacaagtttatacaaaaaggctggttctggttcaaatttggtcgcacaattggaaaacgaagtagcctctttagaaaatgaaaacgaaaccttgaaaaagaaaaacttacataagaaagatttgatcgcttatttggaaaaggaaatcgcaaatttgagaaagaaaattgaagaaggtagtgcaggttctgccgctggttctggtgaatttggttcagctgaagcagccgctaaggaagcagccgctaaagccggttcagctggttccgcagccggttctggtgaattcggttccagttactatcaccatcaccatcatcacttggaatccactagtttatataagaaagcaggttctggttcagcaagaaatgcctacttgagaaagaaaatagctagattaaagaaagataacttgcaattggaaagagatgaacaaaatttggaaaagattatcgccaacttaagagatgaaatcgctagattggaaaatgaagttgcatcccatgaacaaggtagtggt

SEQ ID NO: 182 - DNA, 2472 bp, synthetic construct
  source 1..2472: mol_type = other DNA
atgaaaaactgtgtaatcgtttctgctgttagaactgcaattggttcctttaatggtagtttggcctctacatcagctattgatttgggtgctaccgtcatcaaagctgcaattgaaagagcaaagattgattctcaacatgtcgacgaagtaataatgggtaacgttttgcaagctggtttaggtcaaaatccagcaagacaagccttgttaaaatctggtttagcagaaactgtatgtggtttcacagttaataaggtctgcggttctggtttgaagtcagttgctttagccgctcaagctatacaagcaggtcaagcccaatctatcgtcgctggtggtatggaaaatatgtcattggcaccttatttgttagatgcaaaagccagatcaggttatagattaggtgacggtcaagtatacgacgttattttgagagatggtttaatgtgcgctactcatggttatcacatgggtattacagcagaaaatgttgccaaagaatacggtataaccagagaaatgcaagatgaattggcattacattcccaaagaaaggcagccgctgcaatcgaaagtggtgcttttactgcagaaattgtcccagtaaacgttgtcacaagaaagaaaactttcgttttctcccaagatgaattcccaaaagctaatagtaccgctgaagcattgggtgctttaagacctgcattcgacaaggccggtaccgtaactgccggtaatgcttctggtataaacgatggtgccgctgcattggttatcatggaagaatcagccgctttagcagccggtttgacacctttagctagaattaaatcttatgcatcaggtggtgttccacctgctttgatgggtatgggtccagtccctgctacccaaaaggcattgcaattagccggtttgcaattggctgatatcgacttaatcgaagcaaacgaagcctttgctgcacaattcttggcagttggtaaaaatttgggtttcgactccgaaaaggttaatgtcaacggtggtgccattgctttgggtcatccaataggtgcttcaggtgcaagaatcttggttacattgttgcatgccatgcaagctagagataaaaccttgggtttagctactttgtgtatcggtggtggtcaaggtatcgcaatggttatcgaaagattgaataagttgtctggtggtggtggttctggtggtggtggttctggtggtggtggtagtgcagaagcctggtacaatttgggtaacgcttactacaagcagggtgactaccaaaaggcaatcgaatactaccaaaaggccttggaattagatccaaataacgctgaagcatggtataatttgggtaatgcctattataaacagggtgactatcaaaaagctattgaatattaccaaaaggcattggaattagatcctaataacgccgaagcttggtataatttgggtaacgcctattataagcagggtgactatcaaaaggccatcgaagattaccaaaaggctttggaattggatccaaacaacttgcaagcagaagcctggaagaatttgggtaacgcttattacaaacagggtgactaccaaaaagctattgaatactatcaaaaagccttagaattggatcctaataacgcttctgcatggtacaatttgggtaatgcctactataaacagggtgactaccagaaggctattgaatactaccaaaaagcattagaattggatccaaataacgccaaggcttggtacagacgtggtaatgcctattacaagcagggtgactaccagaaagccatagaagactatcaaaaagccttggaattggatcctaacaacagatccagaagtgctggtggtggtggttctggtggtggtggttctggtggtggtggtgcttcttcatattaccatcaccatcaccatcacttggaatctacatcattatacaaaaaggctggttccggtagtaatgaagttactacattggaaaacgatgccgcttttatcgaaaacgaaaacgcatacttggaaaaggaaatcgccagattaagaaaggaaaaggcagccttgagaaatagattagcccataaaaagggttccgctggtagtgctgcaggttctggtgaatttggttcagctgaagccgctgcaaaagaagccgctgcaaaggcaggttctgccggttcagccgctggttctggtgaattcggttccagttactatcaccatcaccatcatcacttggaatctacttcattatataaaaaggccggttccggtagtcaaaaagtcgctgaattaaagaacagagtagctgttaagttgaacagaaacgaacaattgaaaaataaggtagaagaattgaaaaatagaaacgcctacttaaagaatgaattggcaacattggaaaacgaagtcgctagattggaaaatgatgtagcagaaggttctggt

SEQ ID NO: 183 - DNA, 2337 bp, synthetic construct
  source 1..2337: mol_type = other DNA
atgaaaaaggtttgtgtcattggtgctggtaccatgggttctggtatagcacaagcctttgctgcaaaaggtttcgaagttgtcttgagagatatcaaggacgaattcgttgatagaggtttggacttcatcaataagaacttgtctaagttggttaaaaagggtaaaatcgaagaagctacaaaggtagaaatcttgaccagaatttcaggtactgttgatttgaatatggccgctgattgtgacttggtaatcgaagcagccgttgaaagaatggatattaagaaacaaatcttcgcagatttggacaacatctgcaaacctgaaacaatcttagcctcaaacacctcttcattgtccattactgaagtcgctagtgcaacaaaaagaccagataaggtaataggcatgcatttctttaatccagctcctgttatgaagttggtagaagttattagaggtatagcaacatctcaagaaacctttgacgctgttaaggaaacttcaatagcaatcggtaaagatccagtcgaagtagccgaagctcctggtttcgtagttaacagaatcttgatacctatgatcaacgaagctgttggtatcttggctgaaggtattgcatctgtcgaagatattgacaaagccatgaagttaggtgctaatcacccaatgggtcctttggaattgggtgactttattggtttggacatatgtttagctatcatggacgttttgtattccgaaacaggtgacagtaaatacagaccacataccttgttgaagaaatatgttagagcaggttggttaggtagaaagtctggtaaaggtttctacgattactctaaaaagttgtctggtggtggtggttctggtggtggtggttctggtggtggtggtagtgcagaagcctggtacaatttgggtaacgcttactacaagcagggtgactaccaaaaggccatagaatactaccaaaaggctttggaattggatcctaataacgctgaagcatggtataatttgggtaatgcatattataaacagggtgactatcaaaaggcaatcgaatactaccaaaaggccttggaattagatccaaataacgccgaagcttggtataatttgggtaacgcctattataagcagggtgactatcaaaaagctatcgaagattaccaaaaggcattggaattggatcctaacaacttacaagcagaagcctggaagaatttgggtaacgcatattacaaacagggtgactaccaaaaagccattgaatattatcaaaaagctttggaattggatccaaataacgcttcagcatggtacaatttgggtaatgcctattacaagcagggtgactatcagaaagctattgaatattatcaaaaggctttggaattagatcctaataacgccaaggcttggtacagacgtggtaatgcctattacaagcagggtgactaccagaaggccattgaagactatcaaaaagccttggaattggatccaaacaacagatctagatcagctggtggtggtggttctggtggtggtggttctggtggtggtggtgcttccgaaaatttgtacttccaaggtgaaaacttgtacttccagggtgactccagtgaaagttgttggaattgcggtagaaaagcctccgaaacctgtagtggttgcaacactgctagatattgtggttctttttgccaacacaaagattgggaaaagcatcaccatatttgtggtcaaacattacaagcacaacaaggttctgccggttcagctgcaggttctggtgaatttggttccgctgaagccgctgcaaaagaagccgctgcaaaggcaggttccgccggtagtgccgctggtagtggtgaattcggttctatggcagtttccgaaagtcaattgaagaaaatggtttctaagtacaagtacagagatttgactgttagagaaacagttaacgtcatcactttgtacaaggatttgaagccagtcttggactcatacgtttttaatgatggttcttcaagagaattgatgaacttaactggtacaataccagttccttaccgtggtaacacttacaacatcccaatctgtttgtggttgttagatacatatccttacaatccacctatctgcttcgtcaaaccaacatccagtatgaccattaaaactggtaaacatgttgatgctaacggtaaaatatatttgccatacttacacgaatggaagcatcctcaatcagacttgttgggtttaatccaagtaatgatcgtcgtatttggtgacgaaccacctgttttctctagaccaggttcaggt

SEQ ID NO: 184 - DNA, 2232 bp, synthetic construct
  source 1..2232: mol_type = other DNA
atggaattgaacaacgttatattggaaaaggagggtaaagtcgctgttgtcactataaatagaccaaaggcattgaacgccttgaactctgatacattgaaggaaatggactacgttatcggtgaaattgaaaacgattcagaagtcttagcagtaattttgaccggtgccggtgaaaaatcctttgttgccggtgctgatatcagtgaaatgaaggaaatgaacactatcgaaggtagaaagttcggtatcttgggtaacaaggttttcagaagattggaattgttggaaaagcctgttatagctgcagtcaatggtttcgctttgggtggtggttgtgaaatcgcaatgtcctgcgatattagaatagcttcttcaaacgcaagatttggtcaaccagaagtcggtttaggtattacacctggtttcggtggtacccaaagattatctagattggttggtatgggtatggccaagcaattgatttttactgctcaaaacatcaaggctgatgaagcattgagaatcggtttggttaataaggtagttgaaccatctgaattgatgaacaccgccaaggaaatcgctaataagattgtttctaatgctccagttgctgtcaagttgagtaagcaagctataaatcgtggtatgcaatgtgatatcgacactgcattggccttcgaatctgaagcatttggtgaatgcttctcaacagaagatcaaaaagacgcaatgaccgcctttatcgaaaagagaaagatagaaggtttcaaaaacagaaagttatctggtggtggtggttctggtggtggtggttctggtggtggtggtagtgctgaagcatggtacaatttgggtaacgcttactacaagcagggtgactaccaaaaggcaatcgaatactaccaaaaggccttggaattggacccaaataacgccgaagcttggtataatttgggtaatgcctattataaacagggtgactatcaaaaagctatagaatactaccaaaaggcattggaattggaccctaataacgcagaagcctggtataatttgggtaacgcctattataagcagggtgactatcaaaaggccatagaagactaccaaaaggctttggaattggatccaaacaacttacaagctgaagcatggaagaatttgggtaacgcttattacaaacagggtgactaccaaaaagctattgaatattatcaaaaagctttagaattagaccctaataacgcctctgcttggtacaatttgggtaatgcctactataaacagggtgactaccagaaggctattgaatattaccaaaaagctttagaattggatccaaataacgcaaaggcctggtacagacgtggtaatgcctattacaagcagggtgactaccagaaagccattgaagattatcaaaaagctttggaattggatcctaacaacagatccagaagtgctggtggtggtggttctggtggtggtggttctggtggtggtggtgcttctggtccattgggttcccctttgactgcatcaatgttagcttccgcaccacctcaagaacaaaagcaaatgttgggtgaaagattattcccattgatacaagctatgcatcctactttagcaggtaaaatcacaggcatgttgttggaaatcgataactctgaattgttacacatgttagaatccccagaaagtttgagatctaaagttgacgaagccgtagctgttttgcaagctcatcaagcaaaagaagccgctcaaaaggccggttcagctggttccgcagccggtagtggtgaatttggttctgctgaagctgcagccaaagaagctgcagccaaggcaggtagtgccggttctgctgcaggttctggtgaattcggttccaataccaacatgagtgtcccaactgatggtgctgtaactacatctcaaattcctgcatcagaacaagaaactttagttagaccaaagcctttgttgttgaagttgttgaagtcagtaggtgctcaaaaagatacctacactatgaaggaagttttattttatttgggtcaatacatcatgacaaagagattatacgatgaaaagcaacaacatatcgtttactgttcaaacgatttgttgggtgacttgtttggtgtaccatctttctcagttaaggaacacagaaagatctatacaatgatatacagaaatttggtcgtaggttctggt

SEQ ID NO: 185 - DNA, 2469 bp, synthetic construct
  source 1..2469: mol_type = other DNA
atgatcgtaaagccaatggttagaaacaacatctgtttgaacgctcatcctcaaggttgcaaaaagggtgtagaagatcaaatcgaatacaccaaaaagagaatcactgcagaagttaaagccggtgctaaagcacctaagaatgttttggtcttaggttgttccaacggttatggtttggctagtagaataacagctgcatttggttacggtgccgctaccatcggtgtttccttcgaaaaggctggtagtgaaaccaaatatggtactccaggttggtacaataacttggcatttgatgaagcagccaagagagaaggtttatactctgtcactatagatggtgacgctttctcagatgaaatcaaggcacaagttattgaagaagccaaaaagaaaggtataaaattcgatttgatcgtttactccttagcaagtccagtcagaacagatcctgacaccggtataatgcataagtctgttttgaagccattcggtaaaactttcacaggtaaaacagtcgatcctttcaccggtgaattgaaagaaatatctgctgaaccagcaaatgatgaagaagctgcagccacagtaaaagttatgggtggtgaagactgggaaagatggatcaagcaattgtccaaagaaggtttgttggaagaaggttgtatcaccttagcttattcatacattggtcctgaagccactcaagctttgtatagaaaaggtacaatcggtaaagctaaagaacatttggaagccaccgctcacagattaaataaggaaaacccatctatcagagcatttgtttctgtaaataagggtttagttactagagcatccgccgttatcccagtcattcctttgtatttggctagtttgtttaaggttatgaaggaaaagggtaaccatgaaggttgcatagaacaaatcactagattgtacgcagaaagattatacagaaaggatggtacaattccagttgacgaagaaaacagaatcagaatcgatgactgggaattggaagaagatgtccaaaaggcagtatctgccttaatggaaaaagttaccggtgaaaacgctgaatcattgactgatttggcaggttatagacacgactttttagcctctaatggtttcgatgtcgaaggtattaactacgaagcagaagtagaaagattcgacagaattaaattgtctggtggtggtggttctggtggtggtggttctggtggtggtggtagtgctgaagcatggtataatttgggtaacgcttattacaagcagggtgactaccaaaaggccatcgaatactaccaaaaggctttggaattggaccctaataacgccgaagcttggtacaatttgggtaatgcctactataaacagggtgactatcaaaaagcaattgaatattaccaaaaggccttggaattagacccaaataacgcagaagcctggtacaatttgggtaacgcctactataagcagggtgactatcaaaaggctattgaagactaccaaaaggcattggaattagatcctaataacttgcaagctgaagcatggaaaaatttgggtaatgcctattataaacagggtgactaccaaaaagctattgaatactatcaaaaagctttggaattggacccaaataacgcctcagcttggtataatttgggtaatgcatactacaaacagggtgactatcagaaggcaatagaatactatcaaaaagccttagaattggatcctaataacgcaaaagcctggtatagacgtggtaatgcctactacaagcagggtgactatcagaaggcgatagaagattatcaaaaggcattggaattggatccaaacaacagatctagatcagctggtggtggtggttctggtggtggtggttctggtggtggtggtgcttcttcatattaccatcaccatcaccatcacttggaatccacaagtttatataagaaagcaggttctggttcaaatttgttagccactttgagatcaacagctgcagtattggaaaacgaaaaccatgttttggaaaaagaaaaggaaaagttgagaaaggaaaaggaacaattgttgaataagttggaagcctacaaaggttctgctggttcagccgctggttccggtgaattcggtagtgctgaagcagccgctaaggaagcagccgctaaagctggttccgcaggtagtgcagccggttctggtgaatttggttccagttactatcaccatcaccatcatcacttggaatccactagtttatataagaaagctggttctggttcaaagagaatcgcatacttgagaaagaaaatcgctgcattaaagaaagataacgccaacttggaaaaggacatcgctaatttggaaaacgaaatcgaaagattgattaaagaaattaaaacattagaaaatgaagttgcttctcatgaacaaggttcaggt

SEQ ID NO: 186 - DNA, 2487 bp, synthetic construct
  source 1..2487: mol_type = other DNA
atgactagagaagttgtcgtagttagtggtgttagaacagctattggtacctttggtggttctttaaaagatgttgcaccagccgaattgggtgcattagtcgtaagagaagctttggcaagagcccaagtttcaggtgacgatgtcggtcatgttgtcttcggtaacgttatccaaacagaaccaagagatatgtatttgggtagagtagctgcagttaatggtggtgttaccataaacgctcctgcattaactgtcaacagattgtgtggtagtggtttacaagctattgtttctgccgctcaaacaatattgttaggtgacaccgacgttgctatcggtggtggtgctgaatctatgtcaagagccccatacttagctcctgcagccagatggggtgccagaatgggtgacgctggtttggttgacatgatgttgggtgctttgcatgatccattccatagaatccacatgggtgtaactgcagaaaacgttgccaaggaatacgatatctcaagagcacaacaagacgaagctgcattagaatcacacagaagagcatccgccgctattaaagccggttactttaaggatcaaatagttccagtagtttctaaaggtagaaagggtgacgttaccttcgatactgacgaacatgttagacacgacgctactattgatgacatgacaaagttaagacctgttttcgtcaaggaaaatggtactgttacagctggtaatgcatctggtttgaacgatgcagccgctgcagtcgtaatgatggaaagagccgaagctgaaagaagaggtttgaaaccattagctagattggtttcttatggtcatgctggtgtcgatcctaaagcaatgggtataggtccagttcctgctactaagatcgcattggaaagagccggtttacaagtctctgatttggacgtaattgaagccaatgaagcttttgccgctcaagcatgtgccgttacaaaagccttgggtttagatccagctaaggtcaatcctaacggtagtggtatctctttaggtcatccaattggtgcaaccggtgccttgataactgttaaggctttgcacgaattgaacagagtacaaggtagatatgcattagttacaatgtgcatcggtggtggtcaaggtattgcagccatattcgaaagaattaagttgtctggtggtggtggttctggtggtggtggttctggtggtggtggtagtgctgaagcatggtacaatttgggtaacgcttactacaagcagggtgactaccaaaaggcaatcgaatattaccaaaaagccttggaattagacccaaataacgccgaagcttggtataatttgggtaatgcctattataaacagggtgactatcaaaaagctatagaatactaccaaaaggcattggaattagaccctaataacgcagaagcctggtataatttgggtaacgcctattataagcagggtgactatcaaaaggccatagaagactaccaaaaggctttggaattggatccaaacaacttacaagctgaagcatggaagaatttgggtaacgcttattacaaacagggtgactaccaaaaagctattgaatactatcaaaaggctttagaattggaccctaataacgcctctgcttggtacaatttgggtaatgcctactataaacagggtgactaccagaaggctatcgaatattatcaaaaagctttagaattggacccaaataacgcaaaggcctggtacagacgtggtaatgcctattacaagcagggtgactaccagaaagctattgaagattatcaaaaggcattggaattggatcctaacaacagatccagaagtgctggtggtggtggttctggtggtggtggttctggtggtggtggtgcttctgatgttatgtgggaatataagtgggaaaatacaggtgacgctgaattatacggtccttttacttcagcacaaatgcaaacatgggtatccgaaggttatttccctgatggtgtttactgcagaaaattagacccacctggtggtcaattctacaactcaaagagaatagatttcgacttgtacaccggttcagctggttccgctgcaggttctggtgaatttggttccgcagaagccgctgcaaaagaagccgctgcaaaggctggtagtgcaggttctgccgctggtagtggtgaatttggttctgaatcagattccgtcgaattcaataacgctatatcttacgtaaataagattaaaaccagatttttagatcatccagaaatctatagatcattcttagaaatcttgcatacataccaaaaagaacaattgcacaccaagggtagacctttcagaggcatgtccgaagaagaagtctttactgaagtagctaatttgtttagaggtcaagaagatttgttgtcagaattcggtcaattcttgccagaagcaaaaagaggttccggt

SEQ ID NO: 187 - DNA, 2169 bp, synthetic construct
  source 1..2169: mol_type = other DNA
atgggtaaaaattacaagtcattggattccgttgtcgcaagtgactttattgccttgggtataacttctgaagtcgcagaaacattgcatggtagattagccgaaattgtatgtaactacggtgctgcaaccccacaaacttggatcaacatagcaaaccatatcttgtcaccagatttgcctttctccttgcaccaaatgttgttttatggttgctacaaggatttcggtcctgctccacctgcatggattccagaccctgaaaaggttaagtcaactaatttgggtgctttgttagaaaagagaggtaaagaattcttgggtgttaagtacaaggatccaatctcttctttttctcacttccaagaattttctgtcagaaaccctgaagtatactggagaacagttttgatggatgaaatgaaaataagtttctctaaggacccagaatgtatcttgagaagagatgacatcaacaacccaggtggttctgaatggttgccaggtggttatttgaactcagctaaaaattgcttgaacgttaactccaataagaaattgaatgatactatgattgtctggagagatgaaggcaacgatgacttgccattgaataagttgacattggatcaattgagaaagagagtttggttggtcggttacgcattagaagaaatgggtttggaaaaaggttgtgccatagctatcgatatgcctatgcatgtagacgctgtagttatctatttggctattgttttagcaggttacgtcgtagtttctatagctgattcattttccgcaccagaaatctcaactagattgagattatccaaagcaaaggccatattcacacaagatcacatcatcagaggtaaaaagagaatccctttatactcaagagtcgtagaagccaaatccccaatggctatagttatcccttgtagtggttctaacattggtgcagaattaagagatggtgacatatcttgggattactttttggaaagagccaaagaattcaagaattgcgaattcactgccagagaacaaccagttgatgcttacactaacattttgttctccagtggtactacaggtgaaccaaaagcaataccttggacacaagccacccctttaaaggccgctgcagatggttggtcacatttggatattagaaaaggtgacgtcatagtatggccaactaatttgggttggatgatgggtccttggttggtttatgctagtttgttaaatggtgcctctattgctttatacaacggtagtccattggtttctggtttcgctaaatttgtccaagatgcaaaagtaacaatgttgggtgttgtcccttcaatcgttagaagttggaagtctacaaattgtgtctcaggttatgattggtccaccatcagatgcttttcttcatccggtgaagcctctaatgtcgacgaatatttgtggttaatgggtagagctaactacaagccagttatcgaaatgtgtggtggtaccgaaattggtggtgcattctcagccggttcctttttacaagctcaatcattgagttctttttcatcccaatgtatgggttgcacattgtacatcttggataagaacggttacccaatgcctaaaaataagccaggtattggtgaattggctttaggtcctgttatgttcggtgcatctaaaacattgttgaacggtaaccatcacgatgtatacttcaagggtatgccaaccttaaatggtgaagttttgagaagacatggtgacatattcgaattaacctcaaacggttactaccatgcccacggtagagctgatgacactatgaacatcggtggtatcaaaatcagttctatcgaaatcgaaagagtatgtaacgaagttgatgacagagtctttgaaaccactgcaattggtgttccaccattgggtggtggtccagaacaattagtaatctttttcgttttgaaggattctaacgacacaaccatagatttgaaccaattgagattatcttttaacttgggtttacaaaagaaattgaacccattattcaaagttactagagtagttccattgtcatccttacctagaactgctacaaacaagattatgagaagagtcttgagacaacaattcagtcattttgaaggttctggt

SEQ ID NO: 188 - DNA, 2556 bp, synthetic construct
  source 1..2556: mol_type = other DNA
atgaagttatctactaaattgtgttggtgcggtattaagggtagattaagaccacaaaagcaacaacaattgcataacacaaacttgcaaatgaccgaattgaagaaacaaaagactgctgaacaaaagactagaccacaaaacgttggtattaaaggtatccaaatctatatccctacacaatgtgtcaatcaatctgaattggaaaagtttgatggtgtatcacagggtaaatacactatcggtttaggtcaaacaaacatgtctttcgtaaacgatagagaagacatctattctatgtcattgactgttttgtccaagttgataaaaagttacaacatcgatacaaacaagattggtagattggaagttggtaccgaaactttgatcgataagtccaagagtgtcaagtctgtattgatgcaattgttcggtgaaaataccgatgttgaaggtatcgacactttaaatgcttgttatggtggtactaacgcattattcaattcattgaactggatcgaatccaatgcctgggatggtagagatgctattgttgtctgcggtgacatcgctatctatgacaaaggtgctgcaagaccaaccggtggtgcaggtactgttgccatgtggataggtccagatgcacctatcgtttttgactctgtcagagcatcatacatggaacatgcctacgatttctacaaaccagacttcacctccgaatatccttacgttgatggtcacttttctttgacttgttacgtcaaggctttggaccaagtatacaagtcttactctaagaaagcaatatctaagggtttggtttcagatccagctggttccgacgcattaaacgtcttgaagtacttcgattacaacgttttccatgtccctacatgcaagttggttaccaagtcttacggtagattgttgtacaacgatttcagagctaacccacaattgttccctgaagtcgacgctgaattagcaactagagattacgacgaatctttgacagataagaacatcgaaaagactttcgtaaacgttgcaaagccattccacaaagaaagagttgcccaatcattaattgtccctacaaataccggtaacatgtatacagcctcagtttacgccgcttttgcttccttgttaaattatgtaggtagtgatgacttgcaaggtaaaagagttggtttattctcctatggtagtggtttagcagcctctttgtactcttgtaagattgtaggtgacgttcaacacattattaaggaattggacatcactaataagttggctaagagaatcactgaaacaccaaaggattatgaagctgcaatcgaattgagagaaaacgcacatttgaagaaaaatttcaaacctcaaggtagtatagaacacttgcaatctggtgtctactacttaacaaacatcgatgacaaattcagaagatcatacgatgttaaaaagaaattgtctggtggtggtggttctggtggtggtggttctggtggtggtggtagtgctgaagcatggtataatttgggtaacgcttattacaagcagggtgactaccaaaaagcaatcgaatattaccaaaaggccttggaattagacccaaataacgccgaagcttggtacaatttgggtaatgcatactataaacagggtgactatcaaaaggctatcgaatactaccaaaaggcattggaattagaccctaataacgcagaagcctggtacaatttgggtaacgcctactataagcagggtgactatcaaaaagccatagaagactaccaaaaggctttggaattagatccaaataacttgcaagctgaagcatggaaaaatttgggtaatgcctactacaaacagggtgactaccaaaaggcaattgaatattatcaaaaagccttggaattagatcctaataacgcctcagcttggtataatttgggtaatgcctattataagcagggtgactaccagaaagccattgaatattatcaaaaggctttagaattggatccaaataacgcaaaagcctggtatagacgtggtaatgcctactacaagcagggtgactatcagaaggctattgaagattatcaaaaagctttggaattggatccaaacaacagatccagaagtgctggtggtggtggttctggtggtggtggttctggtggtggtggtgcttctttgggtcctttgccacctggttgggaagtaagatccacagttagtggtagaatctatttcgttgatcataacaacagaactacacaattcaccgacccaagattgcacggttctgctggttcagccgctggttctggtgaatttggttccgcagaagcagccgctaaggaagcagccgctaaagccggttccgctggtagtgcagccggtagtggtgaatttggttctggtgctatgggtccattaccacctggttgggaaaagagaacagattctaacggtagagtctacttcgtaaaccataataccagaattactcaatgggaagatcctagatctggttcaggt

SEQ ID NO: 189 - DNA, 2487 bp, synthetic construct
  source 1..2487: mol_type = other DNA
atggtagccgttagaagaaaggctttgtctatcttagccgaagctccagttttggcatcagatagattaccttacaagaactacgattacgacagagtatttggtgcttgttgcgaaaacgttattggttatatgccattgcctgtcggtgtaatcggtccattagttattgatggtacatcttaccatatccctatggcaactacagaaggttgtttggttgcatcagccatgagaggttgcaaggcaattaatgctggtggtggtgctaccactgttttaaccaaagatggtatgactagaggtccagttgtcagatttcctactttgaagagatccggtgcttgtaaaatatggttagatagtgaagaaggtcaaaatgccatcaaaaaggcttttaactccactagtagattcgcaagattgcaacatattcaaacatgcttagccggtgacttgttgtttatgagattcagaacaaccactggtgacgctatgggtatgaatatgatatctaagggtgtcgaatactcattgaagcaaatggtagaagaatacggttgggaagatatggaagtagtttctgtttcaggcaactactgtactgacaaaaagccagctgcaattaactggatagaaggtcgtggtaaatctgtcgtagctgaagcaacaatacctggtgacgttgttagaaaggttttgaaatctgacgtatcagctttggttgaattgaacatcgctaaaaatttggttggttccgccatggctggtagtgtcggtggttttaatgcacatgccgctaacttagttacagcagtcttcttggccttaggtcaagatccagctcaaaacgtagaatcttcaaactgtatcaccttgatgaaagaagttgatggtgacttaagaatatccgttagtatgccatcaatagaagtcggtacaatcggtggtggtaccgtcttggaacctcaaggtgcaatgttagatttgttaggtgttagaggtccacatgcaactgcccctggtacaaatgctagacaattggcaagaattgtcgcttgtgcagtattagctggtgaattgtccttatgcgcagccttggctgcaggtcacttagttcaaagtcatatgacacacaacagaaagttgtctggtggtggtggttctggtggtggtggttctggtggtggtggtagtgccgaagcttggtataatttgggtaacgcatattacaagcagggtgactaccaaaaggccatcgaatactaccaaaaggctttggaattggacccaaataacgcagaagcctggtacaatttgggtaatgcttactataaacagggtgactatcaaaaggcaattgaatattaccaaaaggccttggaattagaccctaataacgctgaagcatggtacaatttgggtaacgcctactataagcagggtgactatcaaaaagctattgaagactaccaaaaggcattggaattagatccaaataacttgcaagccgaagcttggaaaaatttgggtaacgcttactacaaacagggtgactaccaaaaagctattgaatactatcaaaaagctttggaattggaccctaataacgcatctgcctggtataatttgggtaatgcttattataaacagggtgactaccagaaggcaatagaatactatcaaaaagccttggaattagacccaaataacgctaaagcatggtatagacgtggtaatgcttactataagcagggtgactaccagaaagctatagaagattatcaaaaggcattggaattggatcctaacaacagatctagatcagctggtggtggtggttctggtggtggtggttctggtggtggtggtgcttccagttattaccatcaccatcaccatcacttggaatccactagtttatacaaaaaggcaggttcagaatttttcagaagagaaagaaataagatggccgctgcaaaatgtagaaacagaagaagagaattgacagataccttacaagctgaaaccgatcaattggaagacgaaaagtctgcattgcaaactgaaatagccaatttgttgaaggaaaaggaaaagttggaattcattttagccgctcatagaccagcttgcaaaattcctgatgacttgggtttcccagaagaaatgtctttagaaggttccgcaggtagtgcagccggttccggtgaatttggtagtgctgaagctgcagccaaggaagctgcagccaaagctggttctgcaggttcagctgcaggttccggtgaattcggttcttcatactatcaccatcaccatcatcacttggaatctacctcattatacaaaaaggctggttccggtagtcaaaaggttgaatctttgaagcaaaagattgaagaattgaagcaaagaaaagcccaattgaagaatgatatcgctaacttagaaaaggaaatcgcctacgctgaaactggttctggt

SEQ ID NO: 190 - DNA, 3147 bp, synthetic construct
  source 1..3147: mol_type = other DNA
atgagtttaccatttttgacatctgctcctggtaaagttattatattcggtgaacatagtgccgtctataataagccagctgtcgctgcatctgtatcagctttgagaacatacttgttgatctctgaatcttcagcacctgataccatcgaattggatttcccagacatctcattcaatcacaagtggtccattaatgatttcaacgctatcaccgaagaccaagtaaactcacaaaagttggccaaagctcaacaagcaactgatggtttgtcacaagaattagtttccttgttagacccattgttggctcaattgtccgaaagtttccattaccacgccgctttctgtttcttgtacatgttcgtttgtttatgccctcatgctaagaatatcaaattttctttgaagtctactttgccaattggtgcaggtttaggttccagtgcctctatatcagtttccttagcattggccatggcttatttgggtggtttgataggtagtaacgatttggaaaagttgtctgaaaacgacaagcatatcgtcaaccaatgggcattcatcggtgaaaaatgcattcacggtactcctagtggtatagataatgcagttgccacatatggtaacgctttgttattcgaaaaggactctcataacggtaccatcaacactaacaacttcaagttcttggatgactttcctgcaataccaatgatcttgacttacacaagaattccaagatctactaaagatttggtagctagagtcagagtattggttacagaaaagttccctgaagttatgaagccaatcttggatgcaatgggtgaatgtgccttgcaaggtttggaaatcatgacaaagttgtcaaagtgcaagggtactgatgacgaagctgttgaaacaaataacgaattgtacgaacaattgttggaattgatcagaatcaatcatggtttgttagtttcaattggtgtctcccacccaggtttagaattgataaagaacttgtcagatgacttaagaatcggttccacaaaattgaccggtgctggtggtggtggttgttctttgaccttgttaagaagagatatcactcaagaacaaatcgacagttttaaaaagaaattgcaagatgacttctcttacgaaactttcgaaacagatttgggtggtactggttgttgcttgttgtcagctaagaatttgaacaaagatttgaagattaaatccttggttttccaattgttcgaaaataagactacaaccaagcaacaaatcgatgacttgttgttgcctggtaatacaaacttgccatggacctcaaaattatctggtggtggtggttctggtggtggtggttctggtggtggtggtagtgctgaagcatggtataatttgggtaacgcatattacaagcagggtgactaccaaaaggctatcgaatactaccaaaaggcattggaattggaccctaataacgccgaagcttggtacaatttgggtaatgcttactataaacagggtgactatcaaaaggccattgaatattaccaaaaggctttggaattggacccaaataacgcagaagcctggtacaatttgggtaacgcttactataagcagggtgactatcaaaaagcaattgaagactaccaaaaggccttagaattggatcctaataacttgcaagctgaagcatggaaaaatttgggtaacgcttattataaacagggtgactaccaaaaagccattgaatactatcaaaaagcattggaattggatccaaataacgcctctgcttggtataatttgggtaatgcttattataagcagggtgactaccagaaagccatagaatactatcaaaaagctttggaattagaccctaataacgcaaaagcctggtatagacgtggtaatgcttactacaaacagggtgactatcagaaggcaatagaagattatcaaaaagctttagaattagacccaaataacagaagtagatctgctggtggtggtggttctggtggtggtggttctggtggtggtggtgcttctatggaacctgcaatggaaccagaaacattggaagccagaatcaatagagctaccaatcctttgaacaaggaattggattgggcttctattaatggtttctgtgaacaattgaacgaagacttcgaaggtccacctttagcaacaagattattggcccataaaattcaatcaccacaagaatgggaagcaatacaagccttaaccgtcttggaaacttgtatgaagtcctgcggtaaaagattccacgatgaagttggtaaattcagatttttgaacgaattgatcaaggttgtctcacctaagtatttgggtagtagaacatctgaaaaggttaaaaacaagatcttggaattgttgtactcctggaccgtaggtttaccagaagaagttaagatcgctgaagcataccaaatgttgaagaaacaaggtattgttaagtcaggttccgccggtagtgcagccggttctggtgaattcggttctgcagaagctgcagccaaggaagctgcagccaaagctggttcagcaggttccgctgcaggttctggtgaatttggttcaggtgcaatgggttccatggccgaagctgaaggtgaaagtttggaatcttggttaaataaggctacaaatccatcaaacagacaagaagattgggaatatatcattggtttctgtgaccaaatcaataaggaattggaaggtcctcaaatagctgttagattattggcacataagatccaatctccacaagaatgggaagccttacaagctttgactgttttagaagcttgtatgaagaattgcggtagaagatttcacaacgaagtcggtaaattcagatttttgaatgaattaattaaggtagttagtccaaaatacttaggtgacagagtttctgaaaaggttaagaccaaagtcatagaattgttgtactcttggactatggccttgcctgaagaagctaagatcaaagatgcataccatatgttgaagagacaaggtatagtccaatcagatccacctatcccagtagacagaactttgattccatctccaccaccaagacctaaaaatggttccggt

SEQ ID NO: 191 - DNA, 2610 bp, synthetic construct
  source 1..2610: mol_type = other DNA
atgtccgaattaagagcttttagtgcacctggtaaagccttgttagctggtggttatttggttttggatacaaagtacgaagcattcgttgtcggtttgtcagccagaatgcatgcagtcgcccacccttacggttctttacaaggttctgataagttcgaagtaagagtcaagtctaagcaattcaaggacggtgaatggttataccatatatctccaaagtcaggttttattcctgtttccataggtggtagtaaaaatccattcatcgaaaaggttattgcaaacgtcttttcttacttcaagcctaacatggatgactactgtaacagaaacttgttcgtcatcgatatattctctgatgacgcttatcattctcaagaagactcagtaactgaacacagaggtaatagaagattgtcctttcatagtcacagaattgaagaagttccaaaaaccggtttaggttcttcagctggtggtttagtcactgtattgactacagctttagcatcctttttcgttagtgatttggaaaacaacgtagacaagtacagagaagttattcataatttggcacaagtagcccactgccaagcacaaggtaaaatcggttccggttttgatgttgctgcagccgcttatggttcaattagatacagaagattcccacctgctttgatatctaatttgccagatatcggttctgctacatatggttcaaagttggcacatttggttgatgaagaagactggaacatcacaattaaatccaaccatttgcctagtggtttgaccttatggatgggtgacattaagaatggttctgaaactgttaagttggtccaaaaagtaaagaactggtacgattctcatatgccagaatcattgaagatctacacagaattagaccatgctaattccagattcatggatggtttgagtaaattagacagattgcatacccacgatgactactctgatcaaatcttcgaatcattggaaagaaacgactgtacttgccaaaaatacccagaaatcacagaagtaagagatgccgttgctaccataagaagatcttttagaaagatcactaaggaatcaggtgcagatatcgaaccacctgttcaaacatctttgttagatgactgtcaaaccttgaagggtgtcttaacttgcttgattccaggtgctggtggttatgatgcaatagccgtcatcactaaacaagatgtagacttgagagctcaaacagcaaacgataagagattttcaaaggtccaatggttagatgtaacccaagctgactggggtgttagaaaagaaaaggatcctgaaacttacttggacaaaaagttatctggtggtggtggttctggtggtggtggttctggtggtggtggtagtgctgaagcatggtacaatttgggtaacgcatactacaagcagggtgactaccaaaaggccatagaatactaccaaaaggctttggaattggacccaaataacgccgaagcttggtataatttgggtaatgcttattataaacagggtgactatcaaaaggcaatcgaatactaccaaaaggccttggaattagaccctaataacgcagaagcctggtataatttgggtaacgcttattataagcagggtgactatcaaaaagctatcgaagactaccaaaaggcattggaattagatccaaataacttgcaagctgaagcatggaagaatttgggtaacgcttactataaacagggtgactaccaaaaagccattgaatattatcaaaaagctttggaattggatcctaataacgcctctgcttggtacaatttgggtaatgcttactataagcagggtgactatcagaaggctattgaatattatcaaaaggctttagaattggaccctaataacgcaaaggcctggtacagacgtggtaacgcttattacaaacagggtgactaccagaaagctattgaagattatcaaaaggcattggaattggatcctaacaacagatccagaagtgctggtggtggtggttctggtggtggtggttctggtggtggtggtgcttccagttattaccatcaccatcaccatcacttggaatctacatcattatacaaaaaggctggttccggtagtcaaaaggttgaagaattgaaaaataagatagccgaattggaaaacagaaacgctgttaaaaagaacagagtcgcacatttgaaacaagaaatagcctacttgaaggatgaattagcagcccatgaatttgaaggttctgccggttcagctgcaggttctggtgaattcggttcagctgaagccgctgcaaaagaagccgctgcaaaggccggttccgctggtagtgccgctggttctggtgaatttggttcttcatactatcaccatcaccatcatcacttggaatctacttcattatataaaaaggccggttccggtagtttcgaaaacgttacacatgaattcattttggctaccttggaaaacgaaaacgcaaagttaagaagattggaagccaagttggaaagagaattagctagattgagaaatgaagttgcatggttaggttctggt

SEQ ID NO: 192 - DNA, 3033 bp, synthetic construct
  source 1..3033: mol_type = other DNA
atgacagtttataccgcttctgtcaccgcacctgtaaatattgctactttgaaatactggggtaaaagagatactaagttgaatttgccaacaaactcttcaatctcagttacattgtcccaagatgacttaagaaccttgacttctgctgcaactgctcctgaattcgaaagagatacattgtggttgaatggtgaaccacattctatcgacaacgaaagaactcaaaactgtttgagagatttgagacaattgagaaaggaaatggagagtaaggatgcttctttgcctacattgagtcaatggaagttgcacatagtttctgaaaacaacttcccaaccgccgctggtttggcatccagtgcagccggtttcgctgcattagtctctgcaatcgccaagttgtaccaattgccacaaagtacatctgaaatcagtagaatcgctagaaaaggttcaggttccgcatgtagatctttatttggtggttacgtcgcatgggaaatgggtaaagccgaagacggtcatgattcaatggccgtacaaatagctgactcttcagattggcctcaaatgaaagcttgcgtcttggttgtctcagacatcaaaaaggatgtatccagtacacaaggcatgcaattgactgttgcaacatccgaattgtttaaagaaagaatcgaacacgtagttccaaaaagattcgaagtcatgagaaaggctatcgtagaaaaggatttcgccaccttcgctaaggaaactatgatggacagtaactctttccatgcaacttgtttggattcatttccacctattttctatatgaacgacacctcaaagagaataatctcctggtgccacactatcaaccaattctacggtgaaacaatcgttgcttacaccttcgatgcaggtcctaatgccgtcttgtattacttagccgaaaacgaatcaaagttgttcgcttttatatataagttgtttggttccgttccaggttgggataaaaagttcactacagaacaattggaagcttttaatcatcaattcgaatcttcaaactttactgccagagaattggacttagaattgcaaaaggatgtagctagagttatcttgacccaagttggttcaggtcctcaagaaactaacgaatccttgatagatgctaagacaggtttgccaaaagaaaaattgtctggtggtggtggttctggtggtggtggttctggtggtggtggtagtgctgaagcatggtataatttgggtaacgcttattacaagcagggtgactaccaaaaggccatcgaatactaccaaaaggctttggaattggaccctaataacgccgaagcttggtacaatttgggtaatgcctactataaacagggtgactatcaaaaagcaattgaatattaccaaaaggccttggaattggacccaaataacgcagaagcctggtacaatttgggtaacgcctactataagcagggtgactatcaaaaggctatcgaagattaccaaaaggcattagaattggatcctaataacttgcaagctgaagcatggaaaaatttgggtaatgcctattataaacagggtgactaccaaaaagctattgaatactatcaaaaagctttagaattagacccaaataacgcctcagcttggtataatttgggtaatgcatactacaaacagggtgactatcagaaggcaattgaatactatcaaaaggcattagaattagatcctaataacgcaaaagcctggtatagacgtggtaatgcctactacaagcagggtgactatcagaaggcgattgaagactaccaaaaggcattggaattggatccaaacaacagatcaagatccgctggtggtggtggttctggtggtggtggttctggtggtggtggtgcttctgcaatggccgatttggaacaaaaggtattggaaatggaagctagtacatatgacggtgtttttatttggaagatctctgatttcccaagaaaaagacaagaagctgttgcaggtagaatccctgctatttttagtccagcattctacacctctagatacggttacaagatgtgtttgagaatatatttgaatggtgacggtactggtagaggtactcatttgtctttgtttttcgtcgtaatgaagggtcctaatgatgctttgttgagatggccttttaatcaaaaggttaccttgatgttgttggatcaaaacaacagagaacacgttatcgacgcttttagacctgatgtcacttccagttctttccaaagaccagttaatgatatgaacattgcttctggttgtcctttgttttgcccagtctcaaagatggaagctaaaaattcctatgttagagatgacgccatcttcattaaggctatcgttgatttgactggtttaggttcagcaggttccgccgctggttctggtgaatttggttccgccgaagcagccgctaaggaagcagccgctaaagcaggtagtgccggttctgcagccggctctggcgaatttggtagtgcctctattaaattgcaatcatccgacggtgaaatcttcgaagttgatgtcgaaatagcaaagcaatctgttaccataaaaactatgttggaagatttgggtatggatgacgaaggtgacgatgatccagttcctttgccaaatgtcaacgctgcaatattgaagaaagttattcaatggtgcacacatcacaaggacgatccacctccacctgaagacgatgaaaataaggaaaagagaactgacgatattccagtatgggaccaagaattcttgaaggttgatcaaggtacattgttcgaattgatcttggccgctaactatttggacatcaagggtttgttagatgtaacatgtaaaaccgttgctaacatgatcaagggtaaaacaccagaagaaatcagaaagacctttaatattaagaatgatttcactgaagaagaagaagcacaagttagaaaggaaaaccaatggtgcggttctggt

SEQ ID NO: 193 - DNA, 2160 bp, synthetic construct
  source 1..2160: mol_type = other DNA
atgactgctgataataactctatgccacatggtgccgtatcttcatacgctaagttggttcaaaaccaaacacctgaagatatcttggaagaattcccagaaatcatccctttgcaacaaagaccaaacactagatccagtgaaacatccaacgatgaaagtggtgaaacctgtttttcaggtcatgacgaagaacaaattaaattgatgaacgaaaactgcatcgtattggattgggatgacaatgcaataggtgccggtactaagaaagtttgtcatttgatggaaaacatagaaaagggtttgttgcacagagctttctccgtttttatattcaatgaacagggtgaattgttattgcaacaaagagcaacagaaaagatcacctttccagatttgtggactaatacatgttgctctcatcctttgtgcattgatgacgaattaggtttgaagggtaaattggatgacaaaattaagggtgctataactgctgcagtcagaaaattagatcatgaattgggtataccagaagacgaaaccaagactcgtggtaaattccatttcttaaacagaatccactatatggctccatctaacgaaccttggggtgaacatgaaatcgattacatcttattttacaagattaatgcaaaggaaaacttgacagttaacccaaacgttaatgaagtcagagatttcaaatgggtttctcctaatgatttgaagaccatgtttgctgacccatcatataagtttactccttggttcaagatcatctgtgaaaactacttgtttaactggtgggaacaattagatgacttgtctgaagttgaaaacgatagacaaatccatagaatgttgaaattgtctggtggtggtggttctggtggtggtggttctggtggtggtggtagtgccgaagcttggtacaatttgggtaacgcttactacaagcagggtgactaccaaaaggcaatcgaatactaccaaaaggccttggaattggacccaaataacgcagaagcctggtataatttgggtaatgcatattataaacagggtgactatcaaaaggctattgaatattaccaaaaggcattggaattggaccctaataacgctgaagcatggtataatttgggtaacgcctattataagcagggtgactatcaaaaagccatcgaagactaccaaaaggctttggaattggatccaaacaacttacaagccgaagcttggaagaatttgggtaacgcttattacaaacagggtgactaccaaaaagctattgaatactatcaaaaagccttagaattagaccctaataacgcatctgcctggtacaatttgggtaatgcctattacaagcagggtgactatcagaaggctattgaatactaccaaaaagcattggaattggatccaaataacgctaaggcatggtacagacgtggtaatgcctattacaagcagggtgactatcaaaaggcgattgaagattatcaaaaagctttggaattggatcctaacaacagatctagatcagctggtggtggtggttctggtggtggtggttctggtggtggtggtgcttcttcatattaccatcaccatcaccatcacttagaatccacaagtttgtacaaaaaggctggttctggttcaaacaccgttaaggaattaaagaactacatccaagaattggaagaaagaaacgcagaattgaaaaatttgaaggaacatttgaagtttgccaaggctgaattagaattcgaattggccgctcacaaatttgaaggttccgctggtagtgcagccggttccggtgaattcggtagtgcagaagctgcagccaaagaagctgcagccaaggctggttctgcaggttcagctgcaggttctggtgaatttggttccagttactatcaccatcaccatcatcacttagaatccactagtttgtataaaaaggccggttctggttcacaaaaagtcgcacaattaaagaatagagtagcctacaagttgaaggaaaacgctaagttggaaaacattgtcgcaagattagaaaacgataatgccaacttggaaaaagacatcgctaatttggaaaaggatattgcaaacttggaaagagatgttgccagaggttctggt

SEQ ID NO: 194 - DNA, 2403 bp, synthetic construct
  source 1..2403: mol_type = other DNA
atggaagctaagatagatgaattgataaataacgacccagtttggtcttcacaaaacgaatccttgatcagtaagccatacaaccatatcttgttaaaacctggtaaaaatttcagattaaatttgatcgtacaaatcaacagagttatgaatttgcctaaggatcaattggctatcgtttctcaaatagtcgaattgttgcataactccagtttgttgatcgatgacatcgaagataacgcaccattgagaagaggtcaaactacatcccacttaatttggggtgtccctagtactattaataccgcaaactacatgtacttcagagccatgcaattggtatcacaattgaccactaaggaaccattgtaccattggttgatcacaatttttaacgaagaattgattaatttgcacagaggtcaaggtttggatatctattggagagacttcttaccagaaattatacctacccaagaaatgtacttgaacatggtaatgaataagactggtggtttgtttagattgaccttgagattaatggaagctttgtctccatcttcacatcacggtcattcattggttcctttcataaacttgttgggtatcatctatcaaatcagagatgactacttgaatttgaaggatttccaaatgtccagtgaaaagggtttcgcagaagacataactgagggtaaattgtcattcccaatcgtccatgccttaaacttcacaaaaaccaagggtcaaaccgaacaacacaatgaaatcttaagaattttgttattgagaacttctgataaggacataaagttgaagttgatccaaatcttggaattcgataccaactcattggcttacactaagaacttcatcaaccaattggttaacatgattaagaatgataacgaaaataagtacttgccagatttggcctcccatagtgacactgctacaaatttgcacgatgaattgttgtacatcatcgaccatttgtccgaattgaaattatctggtggtggtggttctggtggtggtggttctggtggtggtggtagtgcagaagcctggtacaacttgggtaacgcttactacaagcagggtgactaccaaaaggctatcgaatactaccaaaaggcattggaattagacccaaataacgctgaagcatggtacaacttaggcaacgcatattataaacagggtgactatcaaaaggccatagaatactaccaaaaggctttggaattggaccctaataacgccgaagcttggtacaacttgggtaatgcttattacaagcagggtgactatcaaaaagcaattgaagactaccaaaaagccttggaattagatccaaataacttgcaagcagaagcctggaagaacttaggcaacgcatactataaacagggtgactaccaaaaagccattgaatattatcaaaaagctttggaattagaccctaataacgcttctgcttggtataacttaggcaatgcctattataagcagggtgactatcagaaagctattgaatattatcaaaaggccttggaattggacccaaataacgccaaggcttggtacagacgtggtaacgcatactacaaacagggtgactatcagaaggctatcgaagattatcaaaaagcattagaattagatcctaataacagatctagatcagctggtggtggtggttctggtggtggtggttctggtggtggtggtgcttctttgtgtactatgaaaaagggtccatctggttacggttttaatttgcattctgataagtcaaagcctggtcaattcataagatcagttgatccagactcccctgcagaagccagtggtttgagagctcaagatagaattgtcgaagtaaatggtgtctgcatggaaggtaaacaacacggtgacgttgtttctgctattagagctggtggtgacgaaactaagttattggtagttgacagagaaggttccgccggtagtgctgcaggttctggtgaatttggttcagctgaagccgctgcaaaagaagccgctgcaaaggccggttctgctggttcagccgctggttctggtgaattcggttcttcatccggtgctataatctatacagttgaattgaagagatacggtggtccattaggtattactatatctggtacagaagaaccattcgatcctatcatcatcagttctttgactaagggtggtttagctgaaagaacaggtgcaatccatattggtgacagaatattggctatcaattcatccagtttgaaaggtaaaccattgtcagaagctatccacttattgcaaatggcaggtgaaaccgttactttgaaaatcaaaaagcaaacagatgcacaacctgcctcttcaggttctggt

SEQ ID NO: 195 - DNA, 2658 bp, synthetic construct
  source 1..2658: mol_type = other DNA
atgaatcatttgagagccgaaggaccagcttctgtcttagcaataggtactgccaatccagagaacatcttgttacaagatgaatttcctgactattacttcagagttaccaaatccgagcatatgacgcagttgaaggaaaagtttagaaagatctgtgataagagtatgatcagaaagaggaactgcttcttaaacgaagagcatttgaagcaaaatcctagattagtggaacacgagatgcaaacattggatgctaggcaggacatgttagttgtcgaagttcctaaattgggtaaagatgcatgtgccaaagctattaaggaatggggtcaacccaagtctaagataactcatttgatttttactagtgctagcactacagatatgcctggtgcagactatcactgtgccaaactacttggtttatcgccctctgtgaagagagttatgatgtatcaactaggttgctacggtggtggtactgtacttagaatcgctaaagacattgcagaaaataacaagggtgccagggtcttggctgtatgttgcgatattatggcttgcttgtttagaggtccatcagaatccgatttggagctgttggttggtcaagctattttcggtgacggtgctgcagctgttattgttggtgcagaacctgatgagtcagtcggtgaaagaccaatctttgaattggtttctaccggtcaaacgattttaccaaatagtgaaggtacaataggtggtcatatcagagaagctggtttgatattcgatttgcacaaagacgttcctatgctaatatctaacaacatcgaaaagtgtctgatcgaggcttttacccccatcggtatttccgattggaatagtatattctggatcacgcatccaggtggtaaagcaatcctggataaggttgaagagaagctgcatttgaagtctgataagtttgtcgacagcagacatgtattgtcggaacacggtaacatgtcttcatccacagtgctgttcgttatggatgaacttagaaagagatctttggaagagggtaaaagcaccacgggtgacggttttgaatggggtgttctttttggattcggccccggtttgaccgtcgaaagagtagttgttagatctgtaccaattaaatacaagttgtctggtggtggtggttctggtggtggtggttctggtggtggtggtagtgcagaagcctggtacaatttgggtaacgcttactacaagcagggtgactaccagaaggctatcgagtattaccaaaaagcacttgaactggatccaaataacgctgaggcatggtataatttgggcaacgcatattacaaacagggtgactatcaaaaggccatagaatactaccaaaaggctttggagctggatcctaataacgccgaagcttggtacaatttgggaaatgcctattataagcagggtgactatcagaaggcaatagaggactaccaaaaagccctagaacttgatccaaataatttgcaggcagaagcctggaagaatttgggtaatgcttactataaacagggtgactatcagaaagctattgaatactaccaaaaagcactggaattggatcctaataacgcttctgcttggtacaatttgggcaacgcttactacaaacagggtgactaccaaaaagctatcgaatattatcaaaaggctctggaactagatccaaataacgccaaggcttggtatagaaggggaaatgcttattataaacagggtgactaccagaaagcaattgaagactaccaaaaagcccttgaactggatcctaataacagatctagaagcgctggtggtggtggttctggtggtggtggttctggtggtggtggtgcttctggtaacaacttagaaacatacgagtggtacaataagtctatttctagagataaggccgaaaagttactacttgacaccggtaaagaaggtgcttttatggttagagattctagaactccaggtacttatacagtctctgtattcacaaaggctatcatctcagaaaacccatgtatcaagcattaccacatcaaggaaaccaacgactctcctaaaagatattacgtggcagaaaagtacgtttttgattcaatcccactgttgattcaatatcatcagtacaatggtggtggtttggtgactagattgaggtatcctgtttgcggtggtagcgcaggttcggctgcaggatcaggcgaatttggttccgccgaggccgctgcaaaagaagccgctgcaaaggctggatctgcaggctcagccgctggttctggagaatttggttctggttctcatccctggtttttcggtaaaattccaagagcaaaggccgaagaaatgttgtctaaacaaagacacgacggtgcatttttgataagggaaagtgagagcgcacctggtgacttttcgttgtctgttaaattcggtaatgatgtccaacatttcaaggtattgagagatggtgctggtaaatactttttgtgggtcgtaaagttcaattccttgaacgaattagtggattaccatagatcaacttccgttagtaggaaccaacagattttcttgagagatatcgaacaagttccacaacagcctacaggttctgga

SEQ ID NO: 196 - DNA, 2004 bp, synthetic construct
  source 1..2004: mol_type = other DNA
atggctgtaaagcatttgatcgtgttgaaattcaaggatgaaatcacagaggcacaaaaggaagagtttttcaagacctacgttaatttggtcaacataatcccagctatgaaagatgtatactggggtaaagacgtgacccaaaagaataaggaagagggttatacccatatagtagaagtgacgttcgaatcagttgaaactatccaagattacatcatacaccctgctcatgttggctttggtgacgtctacagatccttctgggaaaagttgctgatcttcgattacactccaagaaagaaattgtctggtggtggtggttctggtggtggtggttctggtggtggtggtagtgcagaagcctggtataatttgggaaacgcttattacaaacagggtgactaccaaaaggccatcgagtattaccaaaaagctcttgaactggacccaaataacgctgaggcatggtataatttgggtaacgcatactataagcaaggtgactaccaaaaggcaattgaatattaccaaaaggccttggagttagaccctaataacgccgaagcttggtacaatttgggtaatgcctactataaacagggtgactatcaaaaggctatagaggactaccagaaagcactagaacttgatcccaataacttgcaagcagaagcctggaagaatttgggtaatgcctattataagcaaggtgactatcaaaaagctattgaatactaccaaaaagctctggaattggaccctaataacgcttctgcttggtataatttgggtaatgcatactacaagcaaggtgactaccagaaggcaatagagtattaccaaaaagccttagaactagacccaaataacgccaaggcttggtacagaaggggtaatgcctactacaagcagggtgactaccaaaaagctattgaggactaccaaaaagcacttgaactggatcctaataacagatctagatcagctggtggtggtggttctggtggtggtggttctggtggtggtggtgcttccggtcaagatagaagtgaagccacattgattaaaagattcaaaggagaaggtgttagatacaaggctaagctgatcggtatcgatgaagtttctgctgctagaggtgacaaattgtgtcaagactctatgatgaagctgaagggcgttgtcgcaggtgccagatctaagggtgaacataagcaaaagatatttttgacgatctcattcggtggtattaaaatcttcgatgaaaagactggtgctttacaacatcaccatgcagtacacgaaatctcttacatcgctaaggatatcacagaccatagagcattcggttacgtttgcggtaaagaaggcaatcatagatttgtcgctattaaaaccgcccaagccgctgaaccagtcatcttggatttgagagacttattccagctaatctatgaactaaagcaaagagaagaattggaaaagaaagctggtagcgcaggatcggcagccggtagcggagaatttggttctgctgaggctgcagccaaagaagctgcagccaaggccggctctgctggttcagctgcaggctctggtgaatttggttctggttctcatatgggttctcaattttgggtaacttctcaaaagactgaagcttccgagagatgtggtttgcaaggctcctatattttaagggtggaagccgagaagcttaccctacttacgctgggtgcacagagtcaaatattggaacccctgttgttctggccatatactttattgagaagatacggtagagataaagttatgttcagtttcgaagctggtagaagatgcccaagcggtcctggaacttttacattccagacatcacaaggcaatgatatctttcaggcagttgaagccgctattcaacagcaaaaagcccagggtaaagtcggacaggctcaagacattctaagattggaacaccatcaccatcatcatggttctggt

SEQ ID NO: 197 - DNA, 2400 bp, synthetic construct
  source 1..2400: mol_type = other DNA
atgggtttgtcttcagtttgtacattctctttccaaacgaactaccatactttgctgaaccctcacaacaacaatcccaaaacttctttgctttgctacagacatccaaaaacccctattaagtatagctacaacaatttcccatcgaaacattgtagtactaagagcttccatttgcaaaataagtgctccgaatctttgtctatcgctaagaactcaattagagctgcaactacaaatcagacggaaccacctgagtcggataatcactctgtagccaccaaaattttgaactttggtaaagcttgttggaagctgcaaagaccatacacaataatagccttcacctcctgtgcttgcggtttgtttggtaaagaactgttgcataacacaaatttgatttcgtggtctttgatgttcaaggcatttttctttttggttgcaatcctttgcatcgcctcttttaccacgactattaatcaaatctatgatttgcacatcgacagaattaataagcccgatttgccactagcttcaggtgaaatctccgttaatactgcatggattatgtcaatcattgtcgccttgttcggtttaatcatcacaattaaaatgaaaggtggtccattgtacatcttcggctactgtttcggtatattcggtggtatagtatattccgttccaccttttagatggaaacaaaaccccagtaccgctttcttactaaatttcttggcacatatcatcacaaacttcaccttctactacgcttctagagctgctttgggtttgccattcgaattaagaccatcttttacatttttgctggcttttatgaaatcgatgggttctgcattggccttgattaaagatgcatctgacgttgaaggtgacacaaaattcggcatcagtaccttggctagcaagtacggttctagaaatttgactttgttttgttcaggtatcgtattgttatcctacgtggcagccattttagccggtatcatttggccacaagcttttaacagtaatgtcatgctacttagccacgcaatattggccttctggctgatcttgcagacgagagattttgctttaactaattatgaccctgaggcaggtagaagattctacgaattcatgtggaagctgtactacgctgaatatttggtttacgtctttattaagttgtctggtggtggtggttctggtggtggtggttctggtggtggtggtagtgctgaagcatggtacaacttaggcaacgcatactacaagcagggtgactaccagaaggcaattgagtattaccaaaaagccttagaactagacccaaacaatgccgaggcttggtataacttgggcaatgcttattacaaacagggtgactatcaaaaggctatagaatattaccaaaaggcacttgagctggaccctaacaatgcagaagcctggtataacttaggcaatgcttattacaagcagggtgactatcagaaggccatcgaggactaccaaaaggctttggaactggatccaaacaatttgcaggctgaagcatggaagaatttgggtaacgcttactataaacagggtgactatcagaaagcaatagaatactaccaaaaagccctagaacttgaccctaacaatgcctctgcttggtacaacttgggtaatgcttactataagcagggtgactaccaaaaagctatcgaatattaccaaaaagcactggaattggacccaaacaatgcaaaggcctggtatagaagaggtaacgcctactacaaacagggtgactaccaaaaggctattgaagattaccaaaaggctctggaactagatcctaacaacagatctagatccgctggtggtggtggttctggtggtggtggttctggtggtggtggtgcttctgcagaatacgttagagctctgttcgatttcaacggtaacgatgaagaggacttgccttttaagaaaggtgacattttgagaatcagggacaaaccagaagagcaatggtggaatgctgaagattctgagggtaaaagaggaatgattcctgttccctatgtcgaaaagtacggctcagcaggttccgctgcaggatctggcgaattcggttcagccgaggccgctgcaaaagaagccgctgcaaaggctggaagtgcaggcagcgccgctggttccggagaatttggtagtttgattaaacatatgagagccgaagctttattcgattttactggtaactccaaacttgaactgaatttcaaggcaggtgacgttattttcttgttgagtagaattaataaggactggttggaaggtactgttagaggtgctactggaatattcccactttcttttgtgaaaatcctgaagggctcaggt

SEQ ID NO: 198 - DNA, 1641 bp, synthetic construct
  source 1..1641: mol_type = other DNA
atgaaatgtagcactttttctttctggttcgtttgcaagatcattttctttttcttttcttttaatatccaaacttcgatcgcaaatccaagagaaaacttcttaaagtgtttctcacaatacattcctaataacgccacgaatttgaagctggtatacactcagaacaacccactgtacatgagcgtgctaaactcgacaatccataatttgagattcacttccgatactacacccaaaccattagtaatcgtgacaccttctcatgtttcacacattcaaggaaccatactatgctctaagaaagtcggtttgcagattagaacaaggtctggtggtcatgatagtgaaggcatgtcctacatcagtcaagttccattcgttatcgtcgatttgagaaacatgaggtctatcaaaatagacgttcactcacagacggcttgggtcgaggcaggtgccactttgggagaagtttactactgggtcaacgaaaagaatgaaaatttgtctcttgctgcaggttactgtccaactgtctgcgctggtggtcattttggtggtggtggttatggacctcttatgagaaactacggtttggccgctgataatatcattgacgcacatttggtaaatgtgcacggtaaagttctagatagaaagtcaatgggtgaagatttgttttgggcattgagaggtggtggtgctgaatcctttggtataatcgtagcttggaaaattagattggttgcagtcccaaagtctacaatgttctcagttaagaaaattatggaaatccatgagctggtaaagttggtgaataagtggcaaaacatcgcttacaagtacgataaggacttgctgctaatgacccatttcatcacgagaaacatcactgataaccagggtaaaaataagacagcaatacacacctacttctcttcagttttcttgggtggtgttgattccttagtggatttgatgaataagagtttccctgaactgggtattaagaaaactgattgtagacaattgagctggatcgacacaatcatattctatagtggtgttgtcaactacgatactgacaacttcaacaaagaaatccttctggatagaagtgccggacaaaatggcgctttcaaaattaagttggactacgttaaaaagcctatacccgagtcagtatttgtgcagatccttgaaaaactgtatgaagaggatattggtgctggaatgtacgcattatatccatacggtggtataatggatgaaatctccgagagtgccataccattccctcatagagctggtatcttgtacgaactgtggtacatatgttcttgggaaaaacaagaggataacgaaaagcacttaaactggatcaggaacatctataacttcatgactccttacgtttctaaaaaccccagattggcttatttgaattacagagatttggacataggtatcaacgatcctaaaaatccaaacaactacacacaagcaagaatttggggtgaaaagtacttcggtaaaaatttcgatagattggttaaagtcaagaccttagttgaccccaacaactttttcagaaacgaacaatctattccacctttgcctagacataggcacggctctggt

SEQ ID NO: 199 - DNA, 1644 bp, synthetic construct
  source 1..1644: mol_type = other DNA
atgaactgtagcactttttctttttggttcgtttgcaagataatatttttctttttgtcctttaatatccaaatcagtatcgccaacccacaggaaaactttttaaagtgtttctctgagtacatccccaacaacccagctaaccctaagtttatatatacacaacatgatcagctgtacatgagcgtattgaactcgaccattcaaaatttgagattcacttctgacactacacctaagcccttggtcatagtaactccttctaatgtctcacatatacaagcttctatcttgtgctctaagaaagttggtttgcagattagaacaaggtctggtggtcacgatgcagaaggtttatcctatattagtcaagtcccatttgccatagtagatttgagaaatatgcatactgtgaaagttgacatacactcacagactgcttgggtggaagcaggtgccacattgggagaggtttactactggatcaacgagatgaacgaaaactttagtttcccaggtggttactgtcccacagtcggtgttggtggtcatttttctggtggtggttatggagctttaatgagaaactacggtttggctgcagataatatcattgacgcacatttggtgaacgttgatggtaaagttcttgacagaaaatcaatgggtgaagatttgttttgggctatcagaggtggtggtggtgaaaatttcggtataatcgccgcttgcaaaattaagttggttgtcgtacctagcaaagctactattttctctgtcaaaaagaacatggaaatccatggtttagtaaagttgtttaataagtggcaaaacatcgcatacaagtacgataaggatttgatgcttaccacgcatttcagaactaggaacatcacagataaccatggtaaaaataagactacagttcacggatacttctcttcaattttcttgggtggtgttgattctcttgttgatttgatgaataagtcattcccagaactgggtattaaaaagacagattgtaaggaactgagctggatcgacaccacgattttctatagtggtgtggttaattacaacaccgccaacttcaaaaaggaaatcttgctggatagatccgctggtaaaaagaccgctttttctattaaacttgactacgttaagaaactgatccctgaaactgcaatggttaagatattggagaagctgtacgaagaggaagtcggcgtaggcatgtacgttttgtatccatacggtggtataatggatgagatctccgaaagtgccataccatttcctcatagagctggtatcatgtatgaattatggtacaccgctacgtgggagaagcaagaagataacgagaaacacataaactgggtcagatctgtatacaacttcactacaccttacgtttctcagaacccaagattggcatatttgaactacagagatttggacttgggtaaaaccaaccccgaatctccaaataactatacgcaagcaagaatttggggtgaaaagtacttcggtaaaaatttcaacagattggtgaaggttaagacaaaagccgatccaaacaacttctttagaaacgaacaatctattccaccattgccaccaagacatcatggttccggc

SEQ ID NO: 200 - DNA, 8232 bp, synthetic construct
  source 1..8232: mol_type = other DNA
atgtcagaagagtccttatttgaatcttcaccacaaaagatggagtacgaaatcactaactactctgagagacatacagaattgcctggacacttcatcggtttgaacacagttgacaagctggaagagtctccattgagagatttcgtcaagtcccatggtggtcacaccgtaattagtaagatcttgatagctaacaacggtatcgctgcagtcaaggaaattagatctgttagaaagtgggcatatgaaacctttggtgacgatagaacggtccaattcgtagctatggcaactcctgaagacttggaggccaatgctgaatatatcagaatggccgatcaatacattgaagttccaggtggtacaaataacaataactacgctaatgtcgacttaatagtagatatcgctgaaagagcagacgtggatgccgtttgggctggttggggacatgcttccgaaaaccctttgttacccgaaaaattgtctcagagtaagagaaaagttatttttattggtccacctggaaatgcaatgagatcattaggtgacaagatatccagtactatcgtggcacaatcagccaaagttccatgtattccttggtccggcaccggtgttgacacggtgcatgttgatgaaaagactggtttggtttctgtagatgacgatatctatcagaagggatgttgcacttcacctgaagatggtttgcaaaaggctaagagaatcggtttcccagttatgatcaaggcatcagaaggtggtggtggtaaaggtatcaggcaggtcgaaagagaagaggatttcatcgctctgtaccatcaagccgctaatgaaatacccggttctccaattttcataatgaaactagctggaagggcaagacatttggaagttcagctacttgctgaccaatacggcactaatatttccttgttcggtagagattgcagtgttcaaagaagacatcaaaagattatcgaagaggcaccagtcactatagcaaaagccgaaacatttcacgagatggaaaaggcagctgttagattgggtaaattggtcggatatgtaagtgctggaacagtcgaatatttgtacagccatgacgatggtaaattctactttttggaacttaacccaagattacaagttgagcaccctactacagaaatggtttctggtgttaatttgccagctgcacaactgcagattgctatgggtatccctatgcatagaatcagtgatatcaggactctgtacggtatgaatccacacagcgcttcggagattgacttcgaattcaaaactcaggatgcaactaagaaacaaagaagaccaatcccaaagggtcattgtaccgcttgcagaattacgtccgaagaccccaatgatggttttaaaccatctggtggtactttgcacgaactaaactttagaagctcgtctaatgtctggggttatttctcagtaggcaacaacggtaacatccattctttttcagattcccagttcggtcacatcttcgcatttggagaaaataggcaagcctctagaaagcatatggttgtcgctcttaaagaactgtcaatcagaggtgacttcagaaccacggttgaatacttaattaaactgttggaaactgaagacttcgaagataatacgattactacaggttggttggacgatttgataacccataagatgacggcagaaaaacctgatcccaccttggccgttatctgtggtgccgctacgaaggcctttttagcttctgaagaggctagacataagtacatagaaagcctgcaaaagggtcaggtactatcgaaagacttactacaaacaatgtttcctgtggatttcatccacgaaggtaaaagatacaagtttactgttgctaagtctggcaacgataggtacacgttgttcattaatggtagcaagtgcgacatcattctaagacaactttcagatggtggtttgctgatcgcaattggtggtaaatcacatactatctattggaaggaagaggtcgcagccacaagattgagtgtagacagcatgaccacgttgttagaggttgaaaacgatccaactcaattaagaacaccatctcctggtaaacttgtgaaatttctggttgaaaatggcgagcatataatcaagggtcaaccctacgctgagattgaagttatgaaaatgcagatgccattggtttctcaagaaaacggtatagttcaactacttaaacagcctggatcaaccatagtagctggtgacatcatggcaattatgacgttagacgatccatccaaggtgaaacatgctcttccttttgagggtatgctgcccgatttcggttctccagttattgaaggcactaaaccagcatacaagtttaaatcgttggtttctacactggaaaacatcctaaagggttacgataaccaagttattatgaatgcttctttgcaacagttgatagaagtcttgagaaatcctaagttaccctattcagaatggaaattgcatattagcgctcttcactcgagattgcctgcaaaattggatgaacaaatggaagagctagtcgctagatctttgagaagaggtgctgtatttccagcaaggcaattgagtaagctaattgacatggcagttaaaaacccagaatacaaccctgataaactgttgggtgccgtagtggaaccattggcagatattgcccataagtactctaatggtttagaagctcatgagcactcaatcttcgtgcatttcttggaagagtactacgaggttgaaaaattgttcaacggtcctaacgtcagagaagagaacatcatcctgaagttgagagatgaaaacccaaaggacttggataaagtcgctcttactgtactgagtcatagcaaggtttctgccaaaaataacttaatcctagctatcctgaagcactaccaacctttgtgtaagctgtcatccaaagtttctgcaatattttcaactccattgcaacatatcgtagagcttgaatctaaggctaccgcaaaagtggctttgcaggcaagagaaattttgatccaaggtgctttgccatcagttaaagaaagaacagagcaaatagaacacatcctgaagagtagcgttgtcaaagtcgcatacggttcgtctaatcctaagagatctgaacccgatttgaatatacttaaggatttgatcgattcaaattacgtagtgtttgacgttttactacagttcttaactcatcaagatcctgttgtcacagctgcagccgctcaagtctatataagaagggcctatagagcttacactatcggtgacattagggtacacgaaggcgtgacagttccaatcgtggaatggaaatttcaattgccctccgcagcctttagtaccttcccaacggtaaagtcaaaaatgggtatgaacagagctgtttctgtttctgatttgagctatgtggctaattcgcaatcatcccctttaagagaaggtattctaatggctgtggaccatttggacgatgttgatgaaattttgtctcaatctttggaagttattccaagacaccaaagtagctcgaatggtcccgctccagataggtctggatcttcagcaagtttaagcaacgtagccaatgtgtgtgttgcttccactgagggttttgaaagtgaagaggaaatcttggttagattgagagaaattttggatttgaacaaacaagaattgattaatgcttccatcagaaggatcacattcatgttcggttttaaagatggtagttaccctaagtactacacctttaatggtcccaactacaacgagaacgaaactatcagacatatcgaacctgccttagctttccaattggaactgggtagattgtcaaacttcaacatcaagccaattttcactgataacagaaacatccatgtgtacgaagctgtttcaaagacatccccattagataagagatttttcaccagaggcatcattaggacgggtcacattagagatgatattagcatacaagagtacttgacttcggaagctaacagattaatgtctgacatcctagataatttggaagttaccgacacgtcgaactctgatttgaaccatatctttattaacttcatcgcagtgttcgacatatctcctgaggatgttgaagctgcatttggtggtttcttggaaagattcggtaaaagattgctgagattgagagtctccagtgctgaaatcagaatcatcattaaggatccacaaactggtgcccctgtacccctgagagctttgatcaataatgtttctggttacgtaattaaaaccgagatgtacacggaagtcaagaatgctaagggtgaatgggtattcaagagcttgggtaaacccggctcgatgcacttaagaccaattgcaacaccatatcctgtcaaagaatggttgcaacctaagagatacaaagcccacttaatgggtactacatacgtttacgatttcccagaattgttcagacaggcttcttcttctcaatggaagaatttttccgccgacgttaagctgactgacgatttctttatcagtaacgaactaatcgaggatgaaaatggtgaacttacagaggttgaaagagagccaggagcaaatgccattggcatggtcgcttttaagatcactgtaaagacaccagaatatcctaggggtagacaattcgtagtggttgcaaacgacatcacctttaaaattggttctttcggacctcaagaagatgagtttttcaataaggttactgaatacgctaggaaaagaggtataccaagaatctacttggccgctaattctggagcaaggattggcatggccgaggaaatagtgcctttatttcaggttgcatggaacgacgcagccaacccagataagggattccaatatttgtatttgacttctgagggtatggaaacattgaaaaagttcgataaggaaaactcagtgctgaccgagagaactgttattaatggagaggaaaggttcgtaatcaaaactataatcggttctgaagatggtttgggcgtggagtgtctgagaggtagcggtttgattgctggtgcaacttctagagcttaccatgatatttttactatcacactggtcacttgcagatctgtaggcataggtgcttatttggttagattgggtcaaagggccatccaggtcgaaggccaacctattatattgactggtgcccccgctataaacaaaatgctgggtagagaagtttatacctccaatttgcagttgggtggtacgcaaatcatgtacaataacggtgtttctcatttgacagctgtagacgatttggctggtgtggaaaagattgttgaatggatgtcatatgtgccagctaaaagaaacatgcccgttccaatattggaaactaaggacacatgggatagaccagtagattttacccctacgaatgacgaaacctatgatgtgagatggatgattgagggtagggaaactgagtctggttttgaatacggtttgttcgataagggttctttctttgaaacattatcaggctgggccaagggtgtcgtagtgggaagagctagattgggtggtattcctctaggtgttattggtgtagaaactagaacagttgaaaatttgatccccgcagatccagccaaccctaattctgctgaaactttaattcaggaacctggtcaagtttggcatcccaactcagcttttaaaaccgcacaggccattaatgatttcaacaacggtgaacaattgccaatgatgatactggctaactggagaggtttttctggtggtcaaagggatatgttcaacgaagttttgaagtacggtagttttatcgtcgacgcactggtagattacaagcaacctatcataatatacattccaccaactggtgaattaagaggtggttcttgggttgtcgtagacccaaccattaacgcagatcagatggaaatgtacgccgatgtgaatgctagagcaggtgttttggaaccacaaggaatggttggtattaagtttagaagagaaaaattgctggatactatgaacagattagacgataagtacagggaattgagatctcaactgagcaataagtctttggctccagaagttcatcaacagatctctaagcaactggctgatagggaaagagaattgttgccaatatacggtcagatctcattgcaatttgccgacttacacgataggtcatccagaatggtggctaagggtgttatttcaaaagaattagagtggacagaagctagaagatttttcttttggagattgagaagaagattgaacgaggaatatttgattaaaagattgtcacatcaagttggcgaggcttctagattggaaaagatcgcaaggattagatcttggtatccagcatcagtcgatcacgaagacgatagacaagtagccacttggattgaggaaaattacaagacactggacgataagttgaagggtttaaagctagaatcctttgcccaagacttggctaaaaagattagaagtgaccatgataatgctatcgatggtttgagtgaagttattaaaatgcttagcactgacgataaggaaaaactgttgaagacattgaagaaactgtctggtggtggtggttctggtggtggtggttctggtggtggtggtagtgccgaagcttggtataacttgggaaatgcttattacaagcagggtgactaccaaaaggccatagaatactaccaaaaggctcttgagctggatcctaataacgcagaagcctggtataacttaggcaatgcatactataaacaaggtgactaccaaaaggcaatagagtactaccaaaaggccttggaattagatccaaataacgctgaggcatggtataacttgggcaacgcctactataaacagggtgactatcaaaaggctatagaagattaccagaaggcactagagcttgatcctaataacttgcaagccgaagcttggaagaacttaggaaatgcatactataagcaaggtgactatcaaaaagctattgaatattaccaaaaggctctggagttggatccaaataacgcatctgcttggtacaacttaggcaacgcctactataagcagggtgactatcaaaaagcaattgaatattatcaaaaggccttagagctagatcctaataacgctaaagcatggtataggagaggcaatgcatactacaaacagggtgactaccaaaaagctatagaagattaccaaaaggcacttgaactggatccaaataacagatctagatccgctggtggtggtggttctggtggtggtggttctggtggtggtggtgcttctggttctcatatgagattgggagcccaatctattcagccaaccgctaacttagatagaacggacgatttggtctatttgaatgtaatggaattggttagagctgttttggagttgaaaaatgaactagcacaattgccaccagaaggttacgtggttgtcgtaaagaatgttggtttgactcttagaaagttgataggctcggtcgacgatttgctaccatctttgccatcttcttctagaactgaaatagagggtacacaaaagcttctgaacaaagatttggctgaattgattaataagatgagattggcacaacagaacgccgttacttctttgtctgaggagtgtaagagacaaatgctaactgcttctcatactttggctgttgatgcaaagaacttgttagacgctgtggatcaagcaaaagttttagccaatttggctcacccacctgccgaaggttctgctggatcagctgcaggatccggcgaatttggttctgctgaagccgctgcaaaagaggctgctgcaaaagctggatctgcaggtagtgctgctggtagcggagaatttggttctggtgccatggctactcctggttcagaaaacgttctaccaagagaaccattgattgcaacagccgtgaagttcttgcagaactctagagttagacaatctccattggcaactagaagagcatttttgaaaaagaaaggtttgaccgacgaggaaattgatatggctttccaacagtccggtactgcagccgatgaaccatcttcattgtggggaagtggc

SEQ ID NO: 201 - DNA, 10869 bp, synthetic construct
  source 1..10869: mol_type = other DNA
atgggttctgctggttcagctgcaggttctggtgaattcggttccgctggtagtgccgctggttctggtgaatttggttctgctggttcagcagccggttctggtgaattctcctattaccatcaccatcaccatcacttggaatctacttcattatacaaaaaggctggttccggtagtgccagaaacgcttacttgagaaagaaaattgctagattgaagaaagataatttgcaattggaaagagatgaacaaaacttggaaaagattatcgctaatttgagagatgaaatagcaagattggaaaatgaagttgcttctcatgaacaaggttccgcaggtagtgccgccggttctggtgaatttgctgaagccgctgcaaaggaagccgctgcaaaagcaggttctgccggttcagccgctggtagtggtgaattttcttactatcaccatcaccatcatcacttggaatctacctcattatataaaaaggccggttccggtagtaacttggttgctcaattagaaaatgaagtcgcatcattggaaaacgaaaacgaaactttgaaaaagaaaaacttacataagaaagatttgatcgcttacttagaaaaggaaatagcaaatttgagaaagaaaatagaagaaggttccgctggtagtgcagccggtagtggtgaattcggttctgctgaagctgcagccaaggaagctgcagccaaagaagccgctgctaaagaagctgcagccaaagctggttctgcaggttctgccgcaggttccggtgaatttggttcttcatactatcaccatcaccaccaccacttggaatctacctcattatacaagaaagctggttccggtagtcaaaaggtcgctgaattgaaaaacagagtagctgttaagttgaacagaaacgaacaattgaaaaataaggtagaagaattgaaaaatagaaacgcttacttgaaaaacgaattggcaactttggaaaacgaagtagctagattagaaaacgatgttgctgaaggttctgctggttctgctgctggttctggtgaattcgctgaagcagccgctaaggaagcagccgctaaagccggttccgccggttctgctgcgggctctggtgaattttcctactatcaccatcatcatcaccacttggaatctacatcattatataagaaagccggttccggtagtaatgaagttactacattggaaaacgatgctgcttttattgaaaacgaaaacgcatacttagaaaaggaaatcgctagattgagaaaggaaaaggccgctttgagaaatagattagctcataagaaaggttctgctggtagcgctgctggctctggtgaatttggttccgccgaagccgctgctaaggaagccgctgccaaagaagccgctgccaaggaagccgctgctaaggctggttccgccggttcagctgcaggctctggtgaattcggttctagaccacctaccatctctaatccacctccattgatttccagtgctaaacatccatccgtcggtagtgcaggttccgctgccggctctggcgaatttgccgaagctgctgccaaagaagcagccgctaaagctggttcagcaggttccgctgccggatctggcgaattcaatttcttgcaatctagaccagaacctactgctcctccagaagaaagtttcagatctggtggttcagctggttccgccgcaggatctggcgaatttggttccgcagaagctgccgctaaagaagctgctgcaaaagaagcagccgccaaagaagctgctgcaaaagccggtagtgctggttcagctgccggttccggtgaattcggttcttcaaaaggtaccggtttaaatccaaacgctaaagtttggcaagaaattgctcctggtaacggttctgcaggttccgcagctggttccggtgaattcgccgaggccgctgctaaggaagcagcagccaaagcaggtagtgctggttccgcagctggttcaggtgaattcccagacggtggtaccactttcgaacatttgtggtccagtttagaacctgattctacatacggttctgccggttctgcagcaggcagcggtgaattcggttctgccgaagctgctgctaaagaagctgctgccaaggaagctgctgctaaggaagctgctgccaaagccggtagtgcaggttctgctgccggttcaggtgaatttggttcttcttactatcaccaccaccaccatcacttggaatctacatcattatacaagaaagccggttctggtagtaagagaatcgcatacttaagaaagaaaatcgctgcattgaagaaagataacgcaaacttagaaaaggacatcgctaacttggaaaacgaaatcgaaagattgattaaagaaatcaaaaccttggaaaatgaagttgcatctcatgaacaaggttcagccggttctgcagcgggctccggtgaatttgccgaagctgcagcaaaagaagctgccgctaaggctggtagtgctggttctgctgcaggcagcggtgaattttcttactaccaccatcaccaccatcacttggaatctacttcattatataagaaagcaggttctggtagtaacttgttagcaacattaagatctaccgctgcagtcttggaaaacgaaaaccatgtattggaaaaagaaaaggaaaaattgagaaaggaaaaagaacaattgttgaataagttggaagcttacaaaggttcagcaggttctgcagcgggctctggcgaattcggttccgccgaagctgcagcaaaggaagctgcagctaaagaggccgctgcaaaagaagctgctgccaaagcaggtagtgcaggttccgcagccggctccggcgaatttggttcaccagctacatcccaacatcctccacctccacctggtcatagatctcaagctccttcacatggttccgcaggtagtgcagctggatctggcgaattcgccgaagctgccgctaaggaagctgctgcaaaagctggttccgctggttcagcagcaggttccggtgaattcgaattgaattctttgttgatattgttagaagcagccgaatatttggaaagaagagatagaggttctgccggtagtgctgcaggtagcggcgaatttggttctgcagaagcagccgccaaggaagcagctgcaaaagaagcagcagctaaagaagcagctgcaaaagccggttctgctggttcagccgcaggatctggagaattcggttccagaccacctacaatttccaatccacctccattgatctcttctgccaagcatccatccgttggtagtgcaggttcagctgccggtagtggtgaatttgccgaagccgccgctaaggaagccgccgccaaagcaggttcagccggttccgccgcaggttcaggtgaattcaatttcttgcagtcaagaccagaacctaccgctcctccagaggagagtttcagatctggtggtagtgccggttcagctgccggctctggagaatttggttctgcagaggctgctgccaaggaagccgcagctaaagaagccgctgcgaaagaagccgccgctaaagctggtagtgcaggtagtgctgcgggatctggcgaattcggttcttctaagggtactggtttgaaccctaatgccaaggtctggcaagaaatcgcccctggtaacggttccgcaggttccgccgcaggtagtggtgaattcgccgaggctgccgccaaggaagccgccgctaaggcaggtagtgctggttcagcggccggctctggtgaatttccagacggtggtacaacctttgagcatttgtggtccagtttagaacctgattctacgtacggttctgctggttccgctgcaggatctggcgaattcggttccgcggaagccgccgcaaaagaagccgccgccaaagaagccgccgcaaaggaagccgcagcaaaggcaggtagtgccggctccgccgctggcagtggcgaatttggttcttcatattatcaccatcatcatcatcacttggaatctacttcattatacaagaaagcaggttccggttctaaaagaattgcttacttaagaaagaaaatcgcggctttgaagaaagacaatgctaacttagaaaaagatattgccaacttggaaaatgaaatcgaaagattaattaaggaaattaaaacattggaaaacgaagttgcatcacatgaacaaggttcagctggttccgctgcagggtccggcgaatttgcagaagccgccgccaaggaagccgcagccaaagctggtagtgcaggttctgccgctggctctggcgaattttcttactatcatcatcaccatcaccacttggaatctacttcattatacaagaaagcgggttcaggttctaacttgttagcaactttaagatctacagccgctgttttagaaaatgaaaaccatgtcttagaaaaagaaaaggaaaagttgagaaaggaaaaggaacaattattaaataagttagaagcctacaagggttcagcaggttccgcagcaggctcaggcgaatttggttctgcagaagcggctgctaaggaagctgccgcaaaggaagcagctgctaaggaggccgctgcaaaggctggttctgctggttccgccgcgggctctggagaattcggttccgctttggttgatgacgccgctgattatgaacctccaccttcaaataacgaagaagctttaggttccgctggttccgctgcaggttccggcgagttcgcagaagccgcagcaaaagaagccgcagctaaggcaggtagtgccggatccgccgctggcagtggagaattcagagaattgttcgatgacccatcttacgtcaacgtacaaaatttggataaagctagacaaggttccgccggttctgcagcgggatctggggaatttggttctgcagaagctgccgccaaagaagctgcagctaaagaagccgcagccaaagaagctgctgctaaggccggttctgctggttctgccgcaggatctggggaattcggttccaagaatactaagagtatgaacttcgataacccagtttacagaaagactacagaagaagaaggttcagccggttcagccgccggttccggtgaatttgcagaggctgccgctaaagaggctgccgctaaggccggtagtgctggttctgcagccggctccggagaattcagatctttgccatccacatggattgaaaacaaattatacggcatgtcagaccctaattggggttctgcaggttcagctgcgggatctggtgaattcggttcagcagaagccgcagccaaggaagccgctgcaaaggaggccgctgccaaagaagcagctgctaaggctggttcagccggttccgcagccggcagtggtgaatttggtagtgttgtcgataattctccacctccagctttgcctccaaagaaaagacaatctgctccatctggttcagcaggttcagccgctggttcaggtgaatttgccgaagcagctgccaaggaagctgccgccaaggcgggcagtgcaggttcggctgcggggtctggtgaattcactcaaagatctaaaccacaacctgcagttcctccaagaccatctgctgacttgattttaggttccgccggttccgcagctggctctggcgaattcggttccgctgaggctgccgctaaagaagcggccgctaaagaggcagccgctaaagaggcggccgctaaagcaggttctgcaggttcagcagcaggtagtggtgaatttggttctacagatgaagaaagagaagaaaccgaagaagaagtttatttgttgaactctaccactttgggttcagctggttctgctgcgggttctggcgaatttgcagaagcagctgctaaggaagccgcggcaaaggctggttctgcgggctccgccgcaggttctggtgaatttgatggtaatgtatctggtactcaaagattagactcagctaccgttagaacttattcatgcggttctgccggtagtgcagcgggctctggggaattcggttccgcagaagccgctgcaaaagaagccgctgcaaaagaagccgctgcgaaggaggctgctgctaaggcaggttccgccggtagtgctgcgggttccggcgaatttggttccagttactatcaccatcatcaccaccacttggaatccacaagtttatataagaaagctggttctggttcacaaaaggtagctcaattgaaaaatagagttgcatacaagttgaaggaaaacgctaagttggaaaacatagtagcaagattagaaaacgataacgctaatttggaaaaggacatcgcaaatttggaaaaggatatagctaacttggaaagagatgttgctagaggttctgctggtagtgccgcaggctctggcgaattcgctgaagctgccgctaaagaggctgcggctaaagctggttcagctggttctgcagcggggtctggtgaattttcttattatcaccatcatcaccatcacttggaatccaccagtttatacaagaaagccggctctggttcaaacactgttaaggaattgaaaaattacattcaagaattggaagaaagaaacgctgaattgaaaaatttgaaggaacatttgaagtttgcaaaagccgaattggaattcgaattagcagcccataaatttgaaggttctgccggttctgccgccggatctggagaatttggttctgcggaggctgccgctaaagaagccgccgctaaagaggctgcagctaaggaagctgcagcaaaggctggttctgccggttccgctgccggctccggcgaatttggttcacatgatgactccttgccacatcctcaacaagctacagatgactctggtcatgaatccgacggttccgcaggctctgctgccggctccggcgagtttgctgaagccgctgctaaagaggctgctgctaaagccggttctgccggttcagcagctggatctggagaatttggttccccaaatgctggtagtgttgaacaaaccccaaagaaacctggtttgagaagaagaggtagtgctggttctgccgctggctccggagaatttggttcagccgaagctgcggccaaagaggctgctgcaaaggaggctgcggctaaggaagccgccgctaaagccggttcagctggttccgcggcaggctccggggaatttggttcttcttattatcaccaccaccaccatcacttggaatccactagtttatacaagaaagcaggctctggttcattcgaaaacgtcactcatgaattcattttggcaaccttagaaaacgaaaacgctaagttgagaagattagaagcaaagttggaaagagaattggctagattaagaaatgaagtagcttggttgggttctgcgggctcggccgctggctctggtgaattcgccgaagctgcggccaaggaggctgccgcaaaggccggttctgccggttccgcagcgggatccggcgaattttcttactaccatcatcaccatcaccacttggaatccacaagtttatacaagaaagcgggttctggttcacaaaaagttgaagaattgaaaaataagatagcagaattggaaaacagaaacgctgtaaagaaaaatagagttgcacatttgaagcaagaaatcgcttacttgaaggatgaattagcagcccatgaattcgaaggtagtgccggttccgctgctggctcaggcgaatttggtagtgcagaagctgccgctaaggaggctgccgccaaagaagcagccgcaaaagaagctgccgcaaaagccggttctgcgggctctgctgccggatccggcgaattcggttcagtctccagtactaaattagtatcctttcatgatgacagtgatgaagacttgttacatatcggttctgcaggctcagccgctggctctggagagtttgcagaggcagctgctaaagaagccgccgcaaaggcaggttctgcaggttctgcagctggtagtggtgaattcgctgctgcaaccccaatatctacttttcatgatgactcagacgaagacttgttgcatgtcggttccgcaggttcagcagcgggatccggtgaatttggttcagcagaagctgccgccaaggaggccgctgctaaagaagcagcagccaaggaagcagcagcaaaggccggctctgctggttctgctgccgggtccggcgaatttggttcttcttattaccaccatcatcatcaccacttggaatccactagtttatataagaaagccggttctggttcacaaaaggtggaatcattaaaacaaaagattgaagaattgaagcaaagaaaagcacaattgaaaaatgatattgccaatttggaaaaggaaatcgcttacgcagaaacaggtagtgccggttcagccgcgggctctggtgaattcgcagaagctgccgcaaaagaagctgcagcaaaagccggttctgcaggctctgctgctggctctggcgaattttcctactatcatcatcatcatcatcacttggaatccacaagtttatacaagaaagcgggtagtgaatttttcagaagagaaagaaacaagatggcagccgctaagtgtagaaacagaagaagagaattgactgatacattacaagctgaaacagatcaattagaagacgaaaaatcagctttgcaaaccgaaatcgcaaatttgttgaaagaaaaagaaaaattggaattcattttagcagcccatagaccagcttgcaaaatacctgatgacttgggttttccagaagaaatgtctttagaaggtagtgccggtagtgccgctggctcaggtgaatttggtagtgcagaagctgccgcgaaagaagccgcagctaaagaagctgccgccaaagaggcagccgcaaaggcaggttcagcaggttcagctgccgggtccggggaatttggttcattccaaatgccagctgacactcctccacctgcatatttgccacctgaagatcctatgacaggtagtgccggttctgctgccgggtctggcgaattcgctgaagccgctgctaaggaggctgcagctaaggccggctctgcaggttccgctgcaggttcaggtgaatttgaaagagaatctaacgaagaaccacctccaccttatgaagatccatactggggtaatggtggttctgccggtagtgccgccggctcaggcgaatttggttctgcggaggctgctgcaaaggaagctgcggccaaggaagctgccgcaaaagaggctgctgccaaggccggttcagcaggttcagcagctgggtccggtgaatttggttccagttattatcaccaccatcatcaccacttggaatctacctcattatataagaaagcgggttccggtagtcaaaaagttgcagaattgaaaaacagagttgctgtcaaattaaatagaaatgagcagttgaaaaataaggtcgaggagttgaaaaatagaaacgcatacttgaaaaatgaattggctactttggaaaacgaagtcgcaagattagaaaatgatgtagctgaaggctctgctggttccgcagcgggctcaggtgaattcgccgaagcagccgcaaaggaagctgccgctaaggccggctcagcaggttctgccgccggaagcggtgaattttcttattaccaccaccaccatcaccacttggaatctacttcattatacaagaaagcggggtccggtagtaacgaagtcacaaccttagaaaatgatgcagcctttatagaaaacgaaaatgcctacttagaaaaagaaattgcaagattgagaaaggaaaaagctgcattgagaaacagattagcccacaagaaatcttactatcaccaccatcatcatcacttggaatctacatcattatacaagaaagcgggctccggtagtgctagaaatgcctacttaagaaagaaaatagccagattgaagaaagacaatttgcaattagagagagatgaacagaacttagaaaagattatagccaatttgagagatgaaattgctagattagaaaatgaagtagcttctcatgaacaaggtagtgctggctccgctgccggctccggagaatttgccgaagctgccgccaaggaagccgcggccaaggctggttccgctggttctgctgccggatctggagaattttcctattaccatcatcatcatcatcatttggaatctacatcattatacaagaaagcgggatctggttctaacttggtcgcccaattggagaacgaagtcgcatcattggagaacgaaaacgaaaccttgaagaaaaagaacttacacaaaaaggatttgatagcttacttagaaaaagaaatcgctaatttgagaaagaaaattgaagaaggtagtgcaggttcagccgctggctccggtgaatttggttcagcggaggctgccgctaaggaggcagccgctaaagaagcagccgctaaggaggctgcagcaaaagcaggttccgcaggttctgcagcgggttccggagaatttggttctgaacaaaagttgatctctgaagaagatttggaacaaaagttgatatctgaagaagacttggaacaaaaattaatatcagaagaagatttgggtagtgcaggttcagcagctggttctggagaatttggttcagcaggttctgccgctggaagtggcgaattcggtagtgccggctccgctgctggctctggcgaatttggttctggt

SEQ ID NO: 202 - DNA, 1365 bp, synthetic construct
  source 1..1365: mol_type = other DNA
atgggttctgctggttcagctgcaggttctggtgaatttggttccgcaggtagtgccgctggttctggtgaattcggttctgctggttcagcagccggttctggtgaattttcatattaccatcaccatcaccatcacttggaatccaccagtttatacaaaaaggctggttctggttcagctagaaacgcatatttgagaaagaaaattgctagattgaagaaagataacttgcaattggaaagagatgaacaaaatttggaaaagattatcgccaacttaagagatgaaatagcaagattggaaaacgaagtagcttctcatgaacaaggttccgcaggtagtgcagctggttctggtgaatttgctgaagccgctgcaaaggaagccgctgcaaaagctggttccgctggttcagccgctggttccggtgaattcagttactatcaccatcaccatcatcacttggaatccactagtttatataaaaaggccggttctggttcaaatttggttgctcaattagaaaacgaagtcgcatctttagaaaacgaaaacgaaacattgaaaaagaaaaatttgcataagaaagatttgatcgcttatttggaaaaggaaatcgcaaacttgagaaagaaaatagaagaaggttccgctggttctgctgctggttccggtgaatttggttcagctgaagctgcagccaaggaagctgcagccaaagaagccgctgctaaagaagctgcagccaaagcaggttctgccggttctgccgcaggttccggtgaattcggttcttcagctactagagaattggatgaattgatggcatccttaagtgacttcaagatacaaggtggttccgctggttctgcagccggctctggcgaattcgcagaagcagccgctaaggaagcagccgctaaagctggttctgcaggttctgctgccggttctggtgaattcgatttggctttgtctgaaaactgggcacaagaattcttggctgcaggtgacgctgttgatggttctgctggtagtgctgccggttcaggtgaatttggtagtgctgaagctgctgccaaagaagcagccgctaaagaagctgctgccaaggaagctgccgctaaagcaggttccgccggttctgccgccggctccggcgaatttggttcagattataaggatgacgatgacaaggattacaaagacgatgatgacaaggattataaagatgacgatgacaaaggttccgctggtagtgccgccggctctggagaattcggttctgccggttcagctgccggctccggagaatttggttccgctggtagtgcagccggttcaggtgaattcggttctggt

SEQ ID NO: 203 - DNA, 19104 bp, synthetic construct
  source 1..19104: mol_type = other DNA
atgagtgctaaggcaatttctgaacaaactggtaaagaattgttgtacaagtttatttgtactacatcagccatccaaaatagattcaaatacgctagagttaccccagatactgactgggctagattgttacaagatcatccatggttgttatctcaaaacttggttgtcaaacctgaccaattaattaagagaagaggtaaattgggtttagtaggtgttaatttgacattggatggtgtaaagtcttggttgaaaccaagattaggtcaagaagccacagttggtaaagctaccggtttcttgaaaaatttcttgatcgaaccatttgtccctcattcacaagccgaagaattctatgtatgtatctacgctactagagagggtgactatgttttatttcatcacgaaggtggtgtcgacgtaggtgacgttgacgccaaggctcaaaagttgttggttggtgtcgatgaaaagttgaacccagaagacattaaaaagcatttgttggttcacgcacctgaagataaaaaggaaatattggcctcctttataagtggtttgtttaatttctacgaagatttgtacttcacctacttggaaattaacccattagtagttactaaggatggtgtatatgttttggacttagctgcaaaagttgatgcaacagccgactacatttgtaaggtcaaatggggtgacatcgaatttccacctccattcggtagagaagcttatccagaagaagcctacattgctgatttggacgctaagtctggtgcatcattgaagttgacattgttgaaccctaaaggtagaatttggaccatggttgctggtggtggtgctagtgtcgtatattctgatactatatgcgacttgggtggtgttaacgaattggcaaactacggtgaatactcaggtgccccatccgaacaacaaacatacgattacgctaagaccatcttgtccttaatgactagagaaaagcatcctgatggtaaaatcttgatcatcggtggtagtatcgcaaattttactaacgttgccgctacattcaagggtatcgtcagagctataagagattaccaaggtccattgaaggaacacgaagtaacaatattcgttagaagaggtggtcctaactaccaagaaggtttgagagtcatgggtgaagtaggtaaaaccactggtataccaatccatgtctttggtacagaaacccacatgactgcaatagttggtatggccttaggtcatagaccaatccctaatcaacctccaaccgcagcccacactgcaaatttcttgttaaacgcctctggttcaacttccacaccagctccttctagaacagcaagtttctctgaatcaagagctgatgaagtcgctccagctaagaaagcaaaaccagccatgcctcaagactccgttccaagtcctagatctttgcagggtaaatctactactttgttttctagacatactaaggctatagtatggggtatgcaaacaagagcagttcaaggcatgttggatttcgactatgtttgtagtagagatgaaccatctgttgctgcaatggtctatccttttactggtgaccataagcaaaaattctactggggtcacaaggaaatattgatcccagtttttaagaacatggccgatgctatgagaaaacatcctgaagtcgacgtattgattaacttcgcctcattaagatccgcttacgattctacaatggaaaccatgaactacgctcaaataagaaccatcgctatcattgcagaaggtattccagaagccttgactagaaagttgattaagaaagctgatcaaaaaggtgtcacaataatcggtccagctaccgtaggtggtattaagcctggttgtttcaagatcggtaacactggtggtatgttggataacatattggcatctaagttgtatagaccaggttcagtcgcttacgtatccagaagtggtggtatgtccaacgaattgaacaacatcatcagtagaactacagatggtgtatacgaaggtgttgctattggtggtgacagatacccaggttctacttttatggatcatgtattgagatatcaagacacacctggtgttaaaatgattgttgtcttgggtgaaataggtggtactgaagaatacaagatatgcagaggtatcaaagaaggtagattgacaaagccaatcgtttgttggtgcattggtacttgtgcaacaatgttttcttcagaagttcaattcggtcatgcaggtgcctgcgctaatcaagcttcagaaacagcagttgccaagaaccaagcattaaaagaagccggtgtttttgtccctagatctttcgatgaattaggtgaaatcattcaatcagtctatgaagacttggtagctaatggtgtaattgttccagcacaagaagttcctccacctactgtccctatggattactcttgggcaagagaattgggtttaattagaaagccagctagttttatgacctctatatgtgatgaaagaggtcaagaattgatctatgctggtatgcctattactgaagtattcaaagaagaaatgggtatcggtggtgttttaggtttgttgtggttccaaaagagattgccaaagtactcttgtcaattcattgaaatgtgcttaatggttacagctgatcatggtcctgctgtctcaggtgcacacaataccataatctgcgctagagctggtaaagatttggtttcttctttgacctcaggtttgttaactattggtgacagatttggtggtgcattagacgccgctgcaaagatgttttcaaaagctttcgattccggtataatcccaatggaattcgttaataagatgaaaaaggagggtaaattgataatgggtatcggtcatcgtgttaagtctatcaataaccctgatatgagagtacaaatcttgaaggactatgttagacaacactttccagccacacctttgttagattacgctttggaagttgaaaagattaccacttctaaaaagccaaatttgatcttgaacgttgatggtttaattggtgttgcttttgtcgacatgttgagaaactgtggttccttcactagagaagaagctgatgaatatatcgacattggtgcattgaatggtatctttgttttaggtagatctatgggtttcattggtcattacttggatcaaaagagattaaagcaaggtttgtacagacatccatgggatgacatttcttacgttttacctgaacacatgtcaatgaaattgtctggtggtggtggttctggtggtggtggttctggtggtggtggtagtgccgaagcttggtacaatttgggtaacgcatactacaagcagggtgactaccaaaaggcaattgaatattaccaaaaggccttggaattagacccaaataacgcagaagcctggtataatttgggtaatgcttattataaacagggtgactatcaaaaggctatcgaatactaccaaaaggcattggaattagaccctaataacgctgaagcatggtataatttgggtaacgcttattataagcagggtgactatcaaaaagccatcgaagactaccaaaaggctttggaattagatccaaataacttacaagccgaagcttggaagaatttgggtaacgcttactataaacagggtgactaccaaaaagcaattgaatactatcaaaaagctttagaattggaccctaataacgcatcagcctggtacaatttgggtaatgcttactataagcagggtgactatcagaaggccattgaatactatcaaaaggctttagaattggatccaaataacgctaaagcatggtacagacgtggtaacgcttattacaaacagggtgactaccagaaagccattgaagattatcaaaaggctttggaattggatcctaacaacagatctagatcagctggtggtggtggttctggtggtggtggttctggtggtggtggtgcttcttcatattaccatcaccatcaccatcacttggaatccacaagtttatacaaaaaggctggttctggttcaaatttggtcgcacaattggaaaacgaagtagcctctttagaaaatgaaaacgaaaccttgaaaaagaaaaacttacataagaaagatttgatcgcttatttggaaaaggaaatcgcaaatttgagaaagaaaattgaagaaggtagtgcaggttctgccgctggttctggtgaatttggttcagctgaagcagccgctaaggaagcagccgctaaagccggttcagctggttccgcagccggttctggtgaattcggttccagttactatcaccatcaccatcatcacttggaatccacaagtttatataagaaagcaggttctggttcagcaagaaatgcctacttgagaaagaaaatagctagattaaagaaagataacttgcaattggaaagagatgaacaaaatttggaaaagattatcgccaacttaagagatgaaatcgctagattggaaaatgaagttgcatcccatgaacaaggtagtggtgctactaacttctctttgttgaagcaagcaggtgacgttgaagaaaatccaggtccaatgaaaaactgtgtaatcgtttctgctgttagaactgcaattggttcctttaatggtagtttggcctctacatcagctattgatttgggtgctaccgtcatcaaagctgcaattgaaagagcaaagattgattctcaacatgtcgacgaagtaataatgggtaacgttttgcaagctggtttaggtcaaaatccagcaagacaagccttgttaaaatctggtttagcagaaactgtatgtggtttcacagttaataaggtctgcggttctggtttgaagtcagttgctttagccgctcaagctatacaagcaggtcaagcccaatctatcgtcgctggtggtatggaaaatatgtcattggcaccttatttgttagatgcaaaagccagatcaggttatagattaggtgacggtcaagtatacgacgttattttgagagatggtttaatgtgcgctactcatggttatcacatgggtattacagcagaaaatgttgccaaagaatacggtataaccagagaaatgcaagatgaattggcattacattcccaaagaaaggcagccgctgcaatcgaaagtggtgcttttactgcagaaattgtcccagtaaacgttgtcacaagaaagaaaactttcgttttctcccaagatgaattcccaaaagctaatagtaccgctgaagcattgggtgctttaagacctgcattcgacaaggccggtaccgtaactgccggtaatgcttctggtataaacgatggtgccgctgcattggttatcatggaagaatcagccgctttagcagccggtttgacacctttagctagaattaaatcttatgcatcaggtggtgttccacctgctttgatgggtatgggtccagtccctgctacccaaaaggcattgcaattagccggtttgcaattggctgatatcgacttaatcgaagcaaacgaagcctttgctgcacaattcttggcagttggtaaaaatttgggtttcgactccgaaaaggttaatgtcaacggtggtgccattgctttgggtcatccaataggtgcttcaggtgcaagaatcttggttacattgttgcatgccatgcaagctagagataaaaccttgggtttagctactttgtgtatcggtggtggtcaaggtatcgcaatggttatcgaaagattgaataagttgtctggtggtggtggttctggtggtggtggttctggtggtggtggtagtgcagaagcctggtacaatttgggtaacgcttactacaagcagggtgactaccaaaaggcaatcgaatactaccaaaaggccttggaattagatccaaataacgctgaagcatggtataatttgggtaatgcctattataaacagggtgactatcaaaaagctattgaatattaccaaaaggcattggaattagatcctaataacgccgaagcttggtataatttgggtaacgcctattataagcagggtgactatcaaaaggccatcgaagattaccaaaaggctttggaattggatccaaacaacttgcaagcagaagcctggaagaatttgggtaacgcttattacaaacagggtgactaccaaaaagctattgaatactatcaaaaagccttagaattggatcctaataacgcttctgcatggtacaatttgggtaatgcctactataaacagggtgactaccagaaggctattgaatactaccaaaaagcattagaattggatccaaataacgccaaggcttggtacagacgtggtaatgcctattacaagcagggtgactaccagaaagccatagaagactatcaaaaagccttggaattggatcctaacaacagatccagaagtgctggtggtggtggttctggtggtggtggttctggtggtggtggtgcttcttcatattaccatcaccatcaccatcacttggaatctacatcattatacaaaaaggctggttccggtagtaatgaagttactacattggaaaacgatgccgcttttatcgaaaacgaaaacgcatacttggaaaaggaaatcgccagattaagaaaggaaaaggcagccttgagaaatagattagcccataaaaagggttccgctggtagtgctgcaggttctggtgaatttggttcagctgaagccgctgcaaaagaagccgctgcaaaggcaggttctgccggttcagccgctggttctggtgaattcggttccagttactatcaccatcaccatcatcacttggaatctacttcattatataaaaaggccggttccggtagtcaaaaagtcgctgaattaaagaacagagtagctgttaagttgaacagaaacgaacaattgaaaaataaggtagaagaattgaaaaatagaaacgcctacttaaagaatgaattggcaacattggaaaacgaagtcgctagattggaaaatgatgtagcagaaggttctggtgctactaacttctctttgttgaagcaagcaggtgacgttgaagaaaatccaggtccaatgaaaaaggtttgtgtcattggtgctggtaccatgggttctggtatagcacaagcctttgctgcaaaaggtttcgaagttgtcttgagagatatcaaggacgaattcgttgatagaggtttggacttcatcaataagaacttgtctaagttggttaaaaagggtaaaatcgaagaagctacaaaggtagaaatcttgaccagaatttcaggtactgttgatttgaatatggccgctgattgtgacttggtaatcgaagcagccgttgaaagaatggatattaagaaacaaatcttcgcagatttggacaacatctgcaaacctgaaacaatcttagcctcaaacacctcttcattgtccattactgaagtcgctagtgcaacaaaaagaccagataaggtaataggcatgcatttctttaatccagctcctgttatgaagttggtagaagttattagaggtatagcaacatctcaagaaacctttgacgctgttaaggaaacttcaatagcaatcggtaaagatccagtcgaagtagccgaagctcctggtttcgtagttaacagaatcttgatacctatgatcaacgaagctgttggtatcttggctgaaggtattgcatctgtcgaagatattgacaaagccatgaagttaggtgctaatcacccaatgggtcctttggaattgggtgactttattggtttggacatatgtttagctatcatggacgttttgtattccgaaacaggtgacagtaaatacagaccacataccttgttgaagaaatatgttagagcaggttggttaggtagaaagtctggtaaaggtttctacgattactctaaaaagttgtctggtggtggtggttctggtggtggtggttctggtggtggtggtagtgcagaagcctggtacaatttgggtaacgcttactacaagcagggtgactaccaaaaggccatagaatactaccaaaaggctttggaattggatcctaataacgctgaagcatggtataatttgggtaatgcatattataaacagggtgactatcaaaaggcaatcgaatactaccaaaaggccttggaattagatccaaataacgccgaagcttggtataatttgggtaacgcctattataagcagggtgactatcaaaaagctatcgaagattaccaaaaggcattggaattggatcctaacaacttacaagcagaagcctggaagaatttgggtaacgcatattacaaacagggtgactaccaaaaagccattgaatattatcaaaaagctttggaattggatccaaataacgcttcagcatggtacaatttgggtaatgcctattacaagcagggtgactatcagaaagctattgaatattatcaaaaggctttggaattagatcctaataacgccaaggcttggtacagacgtggtaatgcctattacaagcagggtgactaccagaaggccattgaagactatcaaaaagccttggaattggatccaaacaacagatctagatcagctggtggtggtggttctggtggtggtggttctggtggtggtggtgcttccgaaaatttgtacttccaaggtgaaaacttgtacttccagggtgactccagtgaaagttgttggaattgcggtagaaaagcctccgaaacctgtagtggttgcaacactgctagatattgtggttctttttgccaacacaaagattgggaaaagcatcaccatatttgtggtcaaacattacaagcacaacaaggttctgccggttcagctgcaggttctggtgaatttggttccgctgaagccgctgcaaaagaagccgctgcaaaggcaggttccgccggtagtgccgctggtagtggtgaattcggttctatggcagtttccgaaagtcaattgaagaaaatggtttctaagtacaagtacagagatttgactgttagagaaacagttaacgtcatcactttgtacaaggatttgaagccagtcttggactcatacgtttttaatgatggttcttcaagagaattgatgaacttaactggtacaataccagttccttaccgtggtaacacttacaacatcccaatctgtttgtggttgttagatacatatccttacaatccacctatctgcttcgtcaaaccaacatccagtatgaccattaaaactggtaaacatgttgatgctaacggtaaaatatatttgccatacttacacgaatggaagcatcctcaatcagacttgttgggtttaatccaagtaatgatcgtcgtatttggtgacgaaccacctgttttctctagaccaggttcaggtgctactaacttctctttgttgaagcaagcaggtgacgttgaagaaaatccaggtccaatggaattgaacaacgttatattggaaaaggagggtaaagtcgctgttgtcactataaatagaccaaaggcattgaacgccttgaactctgatacattgaaggaaatggactacgttatcggtgaaattgaaaacgattcagaagtcttagcagtaattttgaccggtgccggtgaaaaatcctttgttgccggtgctgatatcagtgaaatgaaggaaatgaacactatcgaaggtagaaagttcggtatcttgggtaacaaggttttcagaagattggaattgttggaaaagcctgttatagctgcagtcaatggtttcgctttgggtggtggttgtgaaatcgcaatgtcctgcgatattagaatagcttcttcaaacgcaagatttggtcaaccagaagtcggtttaggtattacacctggtttcggtggtacccaaagattatctagattggttggtatgggtatggccaagcaattgatttttactgctcaaaacatcaaggctgatgaagcattgagaatcggtttggttaataaggtagttgaaccatctgaattgatgaacaccgccaaggaaatcgctaataagattgtttctaatgctccagttgctgtcaagttgagtaagcaagctataaatcgtggtatgcaatgtgatatcgacactgcattggccttcgaatctgaagcatttggtgaatgcttctcaacagaagatcaaaaagacgcaatgaccgcctttatcgaaaagagaaagatagaaggtttcaaaaacagaaagttatctggtggtggtggttctggtggtggtggttctggtggtggtggtagtgctgaagcatggtacaatttgggtaacgcttactacaagcagggtgactaccaaaaggcaatcgaatactaccaaaaggccttggaattggacccaaataacgccgaagcttggtataatttgggtaatgcctattataaacagggtgactatcaaaaagctatagaatactaccaaaaggcattggaattggaccctaataacgcagaagcctggtataatttgggtaacgcctattataagcagggtgactatcaaaaggccatagaagactaccaaaaggctttggaattggatccaaacaacttacaagctgaagcatggaagaatttgggtaacgcttattacaaacagggtgactaccaaaaagctattgaatattatcaaaaagctttagaattagaccctaataacgcctctgcttggtacaatttgggtaatgcctactataaacagggtgactaccagaaggctattgaatattaccaaaaagctttagaattggatccaaataacgcaaaggcctggtacagacgtggtaatgcctattacaagcagggtgactaccagaaagccattgaagattatcaaaaagctttggaattggatcctaacaacagatccagaagtgctggtggtggtggttctggtggtggtggttctggtggtggtggtgcttctggtccattgggttcccctttgactgcatcaatgttagcttccgcaccacctcaagaacaaaagcaaatgttgggtgaaagattattcccattgatacaagctatgcatcctactttagcaggtaaaatcacaggcatgttgttggaaatcgataactctgaattgttacacatgttagaatccccagaaagtttgagatctaaagttgacgaagccgtagctgttttgcaagctcatcaagcaaaagaagccgctcaaaaggccggttcagctggttccgcagccggtagtggtgaatttggttctgctgaagctgcagccaaagaagctgcagccaaggcaggtagtgccggttctgctgcaggttctggtgaattcggttccaataccaacatgagtgtcccaactgatggtgctgtaactacatctcaaattcctgcatcagaacaagaaactttagttagaccaaagcctttgttgttgaagttgttgaagtcagtaggtgctcaaaaagatacctacactatgaaggaagttttattttatttgggtcaatacatcatgacaaagagattatacgatgaaaagcaacaacatatcgtttactgttcaaacgatttgttgggtgacttgtttggtgtaccatctttctcagttaaggaacacagaaagatctatacaatgatatacagaaatttggtcgtaggttctggtgctactaacttctctttgttgaagcaagcaggtgacgttgaagaaaatccaggtccaatgatcgtaaagccaatggttagaaacaacatctgtttgaacgctcatcctcaaggttgcaaaaagggtgtagaagatcaaatcgaatacaccaaaaagagaatcactgcagaagttaaagccggtgctaaagcacctaagaatgttttggtcttaggttgttccaacggttatggtttggctagtagaataacagctgcatttggttacggtgccgctaccatcggtgtttccttcgaaaaggctggtagtgaaaccaaatatggtactccaggttggtacaataacttggcatttgatgaagcagccaagagagaaggtttatactctgtcactatagatggtgacgctttctcagatgaaatcaaggcacaagttattgaagaagccaaaaagaaaggtataaaattcgatttgatcgtttactccttagcaagtccagtcagaacagatcctgacaccggtataatgcataagtctgttttgaagccattcggtaaaactttcacaggtaaaacagtcgatcctttcaccggtgaattgaaagaaatatctgctgaaccagcaaatgatgaagaagctgcagccacagtaaaagttatgggtggtgaagactgggaaagatggatcaagcaattgtccaaagaaggtttgttggaagaaggttgtatcaccttagcttattcatacattggtcctgaagccactcaagctttgtatagaaaaggtacaatcggtaaagctaaagaacatttggaagccaccgctcacagattaaataaggaaaacccatctatcagagcatttgtttctgtaaataagggtttagttactagagcatccgccgttatcccagtcattcctttgtatttggctagtttgtttaaggttatgaaggaaaagggtaaccatgaaggttgcatagaacaaatcactagattgtacgcagaaagattatacagaaaggatggtacaattccagttgacgaagaaaacagaatcagaatcgatgactgggaattggaagaagatgtccaaaaggcagtatctgccttaatggaaaaagttaccggtgaaaacgctgaatcattgactgatttggcaggttatagacacgactttttagcctctaatggtttcgatgtcgaaggtattaactacgaagcagaagtagaaagattcgacagaattaaattgtctggtggtggtggttctggtggtggtggttctggtggtggtggtagtgctgaagcatggtataatttgggtaacgcttattacaagcagggtgactaccaaaaggccatcgaatactaccaaaaggctttggaattggaccctaataacgccgaagcttggtacaatttgggtaatgcctactataaacagggtgactatcaaaaagcaattgaatattaccaaaaggccttggaattagacccaaataacgcagaagcctggtacaatttgggtaacgcctactataagcagggtgactatcaaaaggctattgaagactaccaaaaggcattggaattagatcctaataacttgcaagctgaagcatggaaaaatttgggtaatgcctattataaacagggtgactaccaaaaagctattgaatactatcaaaaagctttggaattggacccaaataacgcctcagcttggtataatttgggtaatgcatactacaaacagggtgactatcagaaggcaatagaatactatcaaaaagccttagaattggatcctaataacgcaaaagcctggtatagacgtggtaatgcctactacaagcagggtgactatcagaaggcgatagaagattatcaaaaggcattggaattggatccaaacaacagatctagatcagctggtggtggtggttctggtggtggtggttctggtggtggtggtgcttcttcatattaccatcaccatcaccatcacttggaatccacaagtttatataagaaagcaggttctggttcaaatttgttagccactttgagatcaacagctgcagtattggaaaacgaaaaccatgttttggaaaaagaaaaggaaaagttgagaaaggaaaaggaacaattgttgaataagttggaagcctacaaaggttctgctggttcagccgctggttccggtgaattcggtagtgctgaagcagccgctaaggaagcagccgctaaagctggttccgcaggtagtgcagccggttctggtgaatttggttccagttactatcaccatcaccatcatcacttggaatccacaagtttatataagaaagctggttctggttcaaagagaatcgcatacttgagaaagaaaatcgctgcattaaagaaagataacgccaacttggaaaaggacatcgctaatttggaaaacgaaatcgaaagattgattaaagaaattaaaacattagaaaatgaagttgcttctcatgaacaaggttcaggtgctactaacttctctttgttgaagcaagcaggtgacgttgaagaaaatccaggtccaatgactagagaagttgtcgtagttagtggtgttagaacagctattggtacctttggtggttctttaaaagatgttgcaccagccgaattgggtgcattagtcgtaagagaagctttggcaagagcccaagtttcaggtgacgatgtcggtcatgttgtcttcggtaacgttatccaaacagaaccaagagatatgtatttgggtagagtagctgcagttaatggtggtgttaccataaacgctcctgcattaactgtcaacagattgtgtggtagtggtttacaagctattgtttctgccgctcaaacaatattgttaggtgacaccgacgttgctatcggtggtggtgctgaatctatgtcaagagccccatacttagctcctgcagccagatggggtgccagaatgggtgacgctggtttggttgacatgatgttgggtgctttgcatgatccattccatagaatccacatgggtgtaactgcagaaaacgttgccaaggaatacgatatctcaagagcacaacaagacgaagctgcattagaatcacacagaagagcatccgccgctattaaagccggttactttaaggatcaaatagttccagtagtttctaaaggtagaaagggtgacgttaccttcgatactgacgaacatgttagacacgacgctactattgatgacatgacaaagttaagacctgttttcgtcaaggaaaatggtactgttacagctggtaatgcatctggtttgaacgatgcagccgctgcagtcgtaatgatggaaagagccgaagctgaaagaagaggtttgaaaccattagctagattggtttcttatggtcatgctggtgtcgatcctaaagcaatgggtataggtccagttcctgctactaagatcgcattggaaagagccggtttacaagtctctgatttggacgtaattgaagccaatgaagcttttgccgctcaagcatgtgccgttacaaaagccttgggtttagatccagctaaggtcaatcctaacggtagtggtatctctttaggtcatccaattggtgcaaccggtgccttgataactgttaaggctttgcacgaattgaacagagtacaaggtagatatgcattagttacaatgtgcatcggtggtggtcaaggtattgcagccatattcgaaagaattaagttgtctggtggtggtggttctggtggtggtggttctggtggtggtggtagtgctgaagcatggtacaatttgggtaacgcttactacaagcagggtgactaccaaaaggcaatcgaatattaccaaaaagccttggaattagacccaaataacgccgaagcttggtataatttgggtaatgcctattataaacagggtgactatcaaaaagctatagaatactaccaaaaggcattggaattagaccctaataacgcagaagcctggtataatttgggtaacgcctattataagcagggtgactatcaaaaggccatagaagactaccaaaaggctttggaattggatccaaacaacttacaagctgaagcatggaagaatttgggtaacgcttattacaaacagggtgactaccaaaaagctattgaatactatcaaaaggctttagaattggaccctaataacgcctctgcttggtacaatttgggtaatgcctactataaacagggtgactaccagaaggctatcgaatattatcaaaaagctttagaattggacccaaataacgcaaaggcctggtacagacgtggtaatgcctattacaagcagggtgactaccagaaagctattgaagattatcaaaaggcattggaattggatcctaacaacagatccagaagtgctggtggtggtggttctggtggtggtggttctggtggtggtggtgcttctgatgttatgtgggaatataagtgggaaaatacaggtgacgctgaattatacggtccttttacttcagcacaaatgcaaacatgggtatccgaaggttatttccctgatggtgtttactgcagaaaattagacccacctggtggtcaattctacaactcaaagagaatagatttcgacttgtacaccggttcagctggttccgctgcaggttctggtgaatttggttccgcagaagccgctgcaaaagaagccgctgcaaaggctggtagtgcaggttctgccgctggtagtggtgaatttggttctgaatcagattccgtcgaattcaataacgctatatcttacgtaaataagattaaaaccagatttttagatcatccagaaatctatagatcattcttagaaatcttgcatacataccaaaaagaacaattgcacaccaagggtagacctttcagaggcatgtccgaagaagaagtctttactgaagtagctaatttgtttagaggtcaagaagatttgttgtcagaattcggtcaattcttgccagaagcaaaaagaggttccggtgctactaacttctctttgttgaagcaagcaggtgacgttgaagaaaatccaggtccaatgggtaaaaattacaagtcattggattccgttgtcgcaagtgactttattgccttgggtataacttctgaagtcgcagaaacattgcatggtagattagccgaaattgtatgtaactacggtgctgcaaccccacaaacttggatcaacatagcaaaccatatcttgtcaccagatttgcctttctccttgcaccaaatgttgttttatggttgctacaaggatttcggtcctgctccacctgcatggattccagaccctgaaaaggttaagtcaactaatttgggtgctttgttagaaaagagaggtaaagaattcttgggtgttaagtacaaggatccaatctcttctttttctcacttccaagaattttctgtcagaaaccctgaagtatactggagaacagttttgatggatgaaatgaaaataagtttctctaaggacccagaatgtatcttgagaagagatgacatcaacaacccaggtggttctgaatggttgccaggtggttatttgaactcagctaaaaattgcttgaacgttaactccaataagaaattgaatgatactatgattgtctggagagatgaaggcaacgatgacttgccattgaataagttgacattggatcaattgagaaagagagtttggttggtcggttacgcattagaagaaatgggtttggaaaaaggttgtgccatagctatcgatatgcctatgcatgtagacgctgtagttatctatttggctattgttttagcaggttacgtcgtagtttctatagctgattcattttccgcaccagaaatctcaactagattgagattatccaaagcaaaggccatattcacacaagatcacatcatcagaggtaaaaagagaatccctttatactcaagagtcgtagaagccaaatccccaatggctatagttatcccttgtagtggttctaacattggtgcagaattaagagatggtgacatatcttgggattactttttggaaagagccaaagaattcaagaattgcgaattcactgccagagaacaaccagttgatgcttacactaacattttgttctccagtggtactacaggtgaaccaaaagcaataccttggacacaagccacccctttaaaggccgctgcagatggttggtcacatttggatattagaaaaggtgacgtcatagtatggccaactaatttgggttggatgatgggtccttggttggtttatgctagtttgttaaatggtgcctctattgctttatacaacggtagtccattggtttctggtttcgctaaatttgtccaagatgcaaaagtaacaatgttgggtgttgtcccttcaatcgttagaagttggaagtctacaaattgtgtctcaggttatgattggtccaccatcagatgcttttcttcatccggtgaagcctctaatgtcgacgaatatttgtggttaatgggtagagctaactacaagccagttatcgaaatgtgtggtggtaccgaaattggtggtgcattctcagccggttcctttttacaagctcaatcattgagttctttttcatcccaatgtatgggttgcacattgtacatcttggataagaacggttacccaatgcctaaaaataagccaggtattggtgaattggctttaggtcctgttatgttcggtgcatctaaaacattgttgaacggtaaccatcacgatgtatacttcaagggtatgccaaccttaaatggtgaagttttgagaagacatggtgacatattcgaattaacctcaaacggttactaccatgcccacggtagagctgatgacactatgaacatcggtggtatcaaaatcagttctatcgaaatcgaaagagtatgtaacgaagttgatgacagagtctttgaaaccactgcaattggtgttccaccattgggtggtggtccagaacaattagtaatctttttcgttttgaaggattctaacgacacaaccatagatttgaaccaattgagattatcttttaacttgggtttacaaaagaaattgaacccattattcaaagttactagagtagttccattgtcatccttacctagaactgctacaaacaagattatgagaagagtcttgagacaacaattcagtcattttgaaggttctggt

SEQ ID NO: 204 - DNA, 18738 bp, synthetic construct
  source 1..18738: mol_type = other DNA
atgaagttatctactaaattgtgttggtgcggtattaagggtagattaagaccacaaaagcaacaacaattgcataacacaaacttgcaaatgaccgaattgaagaaacaaaagactgctgaacaaaagactagaccacaaaacgttggtattaaaggtatccaaatctatatccctacacaatgtgtcaatcaatctgaattggaaaagtttgatggtgtatcacagggtaaatacactatcggtttaggtcaaacaaacatgtctttcgtaaacgatagagaagacatctattctatgtcattgactgttttgtccaagttgataaaaagttacaacatcgatacaaacaagattggtagattggaagttggtaccgaaactttgatcgataagtccaagagtgtcaagtctgtattgatgcaattgttcggtgaaaataccgatgttgaaggtatcgacactttaaatgcttgttatggtggtactaacgcattattcaattcattgaactggatcgaatccaatgcctgggatggtagagatgctattgttgtctgcggtgacatcgctatctatgacaaaggtgctgcaagaccaaccggtggtgcaggtactgttgccatgtggataggtccagatgcacctatcgtttttgactctgtcagagcatcatacatggaacatgcctacgatttctacaaaccagacttcacctccgaatatccttacgttgatggtcacttttctttgacttgttacgtcaaggctttggaccaagtatacaagtcttactctaagaaagcaatatctaagggtttggtttcagatccagctggttccgacgcattaaacgtcttgaagtacttcgattacaacgttttccatgtccctacatgcaagttggttaccaagtcttacggtagattgttgtacaacgatttcagagctaacccacaattgttccctgaagtcgacgctgaattagcaactagagattacgacgaatctttgacagataagaacatcgaaaagactttcgtaaacgttgcaaagccattccacaaagaaagagttgcccaatcattaattgtccctacaaataccggtaacatgtatacagcctcagtttacgccgcttttgcttccttgttaaattatgtaggtagtgatgacttgcaaggtaaaagagttggtttattctcctatggtagtggtttagcagcctctttgtactcttgtaagattgtaggtgacgttcaacacattattaaggaattggacatcactaataagttggctaagagaatcactgaaacaccaaaggattatgaagctgcaatcgaattgagagaaaacgcacatttgaagaaaaatttcaaacctcaaggtagtatagaacacttgcaatctggtgtctactacttaacaaacatcgatgacaaattcagaagatcatacgatgttaaaaagaaattgtctggtggtggtggttctggtggtggtggttctggtggtggtggtagtgctgaagcatggtataatttgggtaacgcttattacaagcagggtgactaccaaaaagcaatcgaatattaccaaaaggccttggaattagacccaaataacgccgaagcttggtacaatttgggtaatgcatactataaacagggtgactatcaaaaggctatcgaatactaccaaaaggcattggaattagaccctaataacgcagaagcctggtacaatttgggtaacgcctactataagcagggtgactatcaaaaagccatagaagactaccaaaaggctttggaattagatccaaataacttgcaagctgaagcatggaaaaatttgggtaatgcctactacaaacagggtgactaccaaaaggcaattgaatattatcaaaaagccttggaattagatcctaataacgcctcagcttggtataatttgggtaatgcctattataagcagggtgactaccagaaagccattgaatattatcaaaaggctttagaattggatccaaataacgcaaaagcctggtatagacgtggtaatgcctactacaagcagggtgactatcagaaggctattgaagattatcaaaaagctttggaattggatccaaacaacagatccagaagtgctggtggtggtggttctggtggtggtggttctggtggtggtggtgcttctttgggtcctttgccacctggttgggaagtaagatccacagttagtggtagaatctatttcgttgatcataacaacagaactacacaattcaccgacccaagattgcacggttctgctggttcagccgctggttctggtgaatttggttccgcagaagcagccgctaaggaagcagccgctaaagccggttccgctggtagtgcagccggtagtggtgaatttggttctggtgctatgggtccattaccacctggttgggaaaagagaacagattctaacggtagagtctacttcgtaaaccataataccagaattactcaatgggaagatcctagatctggttcaggtgctactaacttctctttgttgaagcaagcaggtgacgttgaagaaaatccaggtccaatggtagccgttagaagaaaggctttgtctatcttagccgaagctccagttttggcatcagatagattaccttacaagaactacgattacgacagagtatttggtgcttgttgcgaaaacgttattggttatatgccattgcctgtcggtgtaatcggtccattagttattgatggtacatcttaccatatccctatggcaactacagaaggttgtttggttgcatcagccatgagaggttgcaaggcaattaatgctggtggtggtgctaccactgttttaaccaaagatggtatgactagaggtccagttgtcagatttcctactttgaagagatccggtgcttgtaaaatatggttagatagtgaagaaggtcaaaatgccatcaaaaaggcttttaactccacaagtagattcgcaagattgcaacatattcaaacatgcttagccggtgacttgttgtttatgagattcagaacaaccactggtgacgctatgggtatgaatatgatatctaagggtgtcgaatactcattgaagcaaatggtagaagaatacggttgggaagatatggaagtagtttctgtttcaggcaactactgtactgacaaaaagccagctgcaattaactggatagaaggtcgtggtaaatctgtcgtagctgaagcaacaatacctggtgacgttgttagaaaggttttgaaatctgacgtatcagctttggttgaattgaacatcgctaaaaatttggttggttccgccatggctggtagtgtcggtggttttaatgcacatgccgctaacttagttacagcagtcttcttggccttaggtcaagatccagctcaaaacgtagaatcttcaaactgtatcaccttgatgaaagaagttgatggtgacttaagaatatccgttagtatgccatcaatagaagtcggtacaatcggtggtggtaccgtcttggaacctcaaggtgcaatgttagatttgttaggtgttagaggtccacatgcaactgcccctggtacaaatgctagacaattggcaagaattgtcgcttgtgcagtattagctggtgaattgtccttatgcgcagccttggctgcaggtcacttagttcaaagtcatatgacacacaacagaaagttgtctggtggtggtggttctggtggtggtggttctggtggtggtggtagtgccgaagcttggtataatttgggtaacgcatattacaagcagggtgactaccaaaaggccatcgaatactaccaaaaggctttggaattggacccaaataacgcagaagcctggtacaatttgggtaatgcttactataaacagggtgactatcaaaaggcaattgaatattaccaaaaggccttggaattagaccctaataacgctgaagcatggtacaatttgggtaacgcctactataagcagggtgactatcaaaaagctattgaagactaccaaaaggcattggaattagatccaaataacttgcaagccgaagcttggaaaaatttgggtaacgcttactacaaacagggtgactaccaaaaagctattgaatactatcaaaaagctttggaattggaccctaataacgcatctgcctggtataatttgggtaatgcttattataaacagggtgactaccagaaggcaatagaatactatcaaaaagccttggaattagacccaaataacgctaaagcatggtatagacgtggtaatgcttactataagcagggtgactaccagaaagctatagaagattatcaaaaggcattggaattggatcctaacaacagatctagatcagctggtggtggtggttctggtggtggtggttctggtggtggtggtgcttccagttattaccatcaccatcaccatcacttggaatccacaagtttatacaaaaaggcaggttcagaatttttcagaagagaaagaaataagatggccgctgcaaaatgtagaaacagaagaagagaattgacagataccttacaagctgaaaccgatcaattggaagacgaaaagtctgcattgcaaactgaaatagccaatttgttgaaggaaaaggaaaagttggaattcattttagccgctcatagaccagcttgcaaaattcctgatgacttgggtttcccagaagaaatgtctttagaaggttccgcaggtagtgcagccggttccggtgaatttggtagtgctgaagctgcagccaaggaagctgcagccaaagctggttctgcaggttcagctgcaggttccggtgaattcggttcttcatactatcaccatcaccatcatcacttggaatctacctcattatacaaaaaggctggttccggtagtcaaaaggttgaatctttgaagcaaaagattgaagaattgaagcaaagaaaagcccaattgaagaatgatatcgctaacttagaaaaggaaatcgcctacgctgaaactggttctggtgctactaacttctctttgttgaagcaagcaggtgacgttgaagaaaatccaggtccaatgagtttaccatttttgacatctgctcctggtaaagttattatattcggtgaacatagtgccgtctataataagccagctgtcgctgcatctgtatcagctttgagaacatacttgttgatctctgaatcttcagcacctgataccatcgaattggatttcccagacatctcattcaatcacaagtggtccattaatgatttcaacgctatcaccgaagaccaagtaaactcacaaaagttggccaaagctcaacaagcaactgatggtttgtcacaagaattagtttccttgttagacccattgttggctcaattgtccgaaagtttccattaccacgccgctttctgtttcttgtacatgttcgtttgtttatgccctcatgctaagaatatcaaattttctttgaagtctactttgccaattggtgcaggtttaggttccagtgcctctatatcagtttccttagcattggccatggcttatttgggtggtttgataggtagtaacgatttggaaaagttgtctgaaaacgacaagcatatcgtcaaccaatgggcattcatcggtgaaaaatgcattcacggtactcctagtggtatagataatgcagttgccacatatggtaacgctttgttattcgaaaaggactctcataacggtaccatcaacactaacaacttcaagttcttggatgactttcctgcaataccaatgatcttgacttacacaagaattccaagatctactaaagatttggtagctagagtcagagtattggttacagaaaagttccctgaagttatgaagccaatcttggatgcaatgggtgaatgtgccttgcaaggtttggaaatcatgacaaagttgtcaaagtgcaagggtactgatgacgaagctgttgaaacaaataacgaattgtacgaacaattgttggaattgatcagaatcaatcatggtttgttagtttcaattggtgtctcccacccaggtttagaattgataaagaacttgtcagatgacttaagaatcggttccacaaaattgaccggtgctggtggtggtggttgttctttgaccttgttaagaagagatatcactcaagaacaaatcgacagttttaaaaagaaattgcaagatgacttctcttacgaaactttcgaaacagatttgggtggtactggttgttgcttgttgtcagctaagaatttgaacaaagatttgaagattaaatccttggttttccaattgttcgaaaataagactacaaccaagcaacaaatcgatgacttgttgttgcctggtaatacaaacttgccatggacctcaaaattatctggtggtggtggttctggtggtggtggttctggtggtggtggtagtgctgaagcatggtataatttgggtaacgcatattacaagcagggtgactaccaaaaggctatcgaatactaccaaaaggcattggaattggaccctaataacgccgaagcttggtacaatttgggtaatgcttactataaacagggtgactatcaaaaggccattgaatattaccaaaaggctttggaattggacccaaataacgcagaagcctggtacaatttgggtaacgcttactataagcagggtgactatcaaaaagcaattgaagactaccaaaaggccttagaattggatcctaataacttgcaagctgaagcatggaaaaatttgggtaacgcttattataaacagggtgactaccaaaaagccattgaatactatcaaaaagcattggaattggatccaaataacgcctctgcttggtataatttgggtaatgcttattataagcagggtgactaccagaaagccatagaatactatcaaaaagctttggaattagaccctaataacgcaaaagcctggtatagacgtggtaatgcttactacaaacagggtgactatcagaaggcaatagaagattatcaaaaagctttagaattagacccaaataacagaagtagatctgctggtggtggtggttctggtggtggtggttctggtggtggtggtgcttctatggaacctgcaatggaaccagaaacattggaagccagaatcaatagagctaccaatcctttgaacaaggaattggattgggcttctattaatggtttctgtgaacaattgaacgaagacttcgaaggtccacctttagcaacaagattattggcccataaaattcaatcaccacaagaatgggaagcaatacaagccttaaccgtcttggaaacttgtatgaagtcctgcggtaaaagattccacgatgaagttggtaaattcagatttttgaacgaattgatcaaggttgtctcacctaagtatttgggtagtagaacatctgaaaaggttaaaaacaagatcttggaattgttgtactcctggaccgtaggtttaccagaagaagttaagatcgctgaagcataccaaatgttgaagaaacaaggtattgttaagtcaggttccgccggtagtgcagccggttctggtgaattcggttctgcagaagctgcagccaaggaagctgcagccaaagctggttcagcaggttccgctgcaggttctggtgaatttggttcaggtgcaatgggttccatggccgaagctgaaggtgaaagtttggaatcttggttaaataaggctacaaatccatcaaacagacaagaagattgggaatatatcattggtttctgtgaccaaatcaataaggaattggaaggtcctcaaatagctgttagattattggcacataagatccaatctccacaagaatgggaagccttacaagctttgactgttttagaagcttgtatgaagaattgcggtagaagatttcacaacgaagtcggtaaattcagatttttgaatgaattaattaaggtagttagtccaaaatacttaggtgacagagtttctgaaaaggttaagaccaaagtcatagaattgttgtactcttggactatggccttgcctgaagaagctaagatcaaagatgcataccatatgttgaagagacaaggtatagtccaatcagatccacctatcccagtagacagaactttgattccatctccaccaccaagacctaaaaatggttccggtgctactaacttctctttgttgaagcaagcaggtgacgttgaagaaaatccaggtccaatgtccgaattaagagcttttagtgcacctggtaaagccttgttagctggtggttatttggttttggatacaaagtacgaagcattcgttgtcggtttgtcagccagaatgcatgcagtcgcccacccttacggttctttacaaggttctgataagttcgaagtaagagtcaagtctaagcaattcaaggacggtgaatggttataccatatatctccaaagtcaggttttattcctgtttccataggtggtagtaaaaatccattcatcgaaaaggttattgcaaacgtcttttcttacttcaagcctaacatggatgactactgtaacagaaacttgttcgtcatcgatatattctctgatgacgcttatcattctcaagaagactcagtaactgaacacagaggtaatagaagattgtcctttcatagtcacagaattgaagaagttccaaaaaccggtttaggttcttcagctggtggtttagtcactgtattgactacagctttagcatcctttttcgttagtgatttggaaaacaacgtagacaagtacagagaagttattcataatttggcacaagtagcccactgccaagcacaaggtaaaatcggttccggttttgatgttgctgcagccgcttatggttcaattagatacagaagattcccacctgctttgatatctaatttgccagatatcggttctgctacatatggttcaaagttggcacatttggttgatgaagaagactggaacatcacaattaaatccaaccatttgcctagtggtttgaccttatggatgggtgacattaagaatggttctgaaactgttaagttggtccaaaaagtaaagaactggtacgattctcatatgccagaatcattgaagatctacacagaattagaccatgctaattccagattcatggatggtttgagtaaattagacagattgcatacccacgatgactactctgatcaaatcttcgaatcattggaaagaaacgactgtacttgccaaaaatacccagaaatcacagaagtaagagatgccgttgctaccataagaagatcttttagaaagatcactaaggaatcaggtgcagatatcgaaccacctgttcaaacatctttgttagatgactgtcaaaccttgaagggtgtcttaacttgcttgattccaggtgctggtggttatgatgcaatagccgtcatcactaaacaagatgtagacttgagagctcaaacagcaaacgataagagattttcaaaggtccaatggttagatgtaacccaagctgactggggtgttagaaaagaaaaggatcctgaaacttacttggacaaaaagttatctggtggtggtggttctggtggtggtggttctggtggtggtggtagtgctgaagcatggtacaatttgggtaacgcatactacaagcagggtgactaccaaaaggccatagaatactaccaaaaggctttggaattggacccaaataacgccgaagcttggtataatttgggtaatgcttattataaacagggtgactatcaaaaggcaatcgaatactaccaaaaggccttggaattagaccctaataacgcagaagcctggtataatttgggtaacgcttattataagcagggtgactatcaaaaagctatcgaagactaccaaaaggcattggaattagatccaaataacttgcaagctgaagcatggaagaatttgggtaacgcttactataaacagggtgactaccaaaaagccattgaatattatcaaaaagctttggaattggatcctaataacgcctctgcttggtacaatttgggtaatgcttactataagcagggtgactatcagaaggctattgaatattatcaaaaggctttagaattggaccctaataacgcaaaggcctggtacagacgtggtaacgcttattacaaacagggtgactaccagaaagctattgaagattatcaaaaggcattggaattggatcctaacaacagatccagaagtgctggtggtggtggttctggtggtggtggttctggtggtggtggtgcttccagttattaccatcaccatcaccatcacttggaatctacatcattatacaaaaaggctggttccggtagtcaaaaggttgaagaattgaaaaataagatagccgaattggaaaacagaaacgctgttaaaaagaacagagtcgcacatttgaaacaagaaatagcctacttgaaggatgaattagcagcccatgaatttgaaggttctgccggttcagctgcaggttctggtgaattcggttcagctgaagccgctgcaaaagaagccgctgcaaaggccggttccgctggtagtgccgctggttctggtgaatttggttcttcatactatcaccatcaccatcatcacttggaatctacttcattatataaaaaggccggttccggtagtttcgaaaacgttacacatgaattcattttggctaccttggaaaacgaaaacgcaaagttaagaagattggaagccaagttggaaagagaattagctagattgagaaatgaagttgcatggttaggttctggtgctactaacttctctttgttgaagcaagcaggtgacgttgaagaaaatccaggtccaatgacagtttataccgcttctgtcaccgcacctgtaaatattgctactttgaaatactggggtaaaagagatactaagttgaatttgccaacaaactcttcaatctcagttacattgtcccaagatgacttaagaaccttgacttctgctgcaactgctcctgaattcgaaagagatacattgtggttgaatggtgaaccacattctatcgacaacgaaagaactcaaaactgtttgagagatttgagacaattgagaaaggaaatggagagtaaggatgcttctttgcctacattgagtcaatggaagttgcacatagtttctgaaaacaacttcccaaccgccgctggtttggcatccagtgcagccggtttcgctgcattagtctctgcaatcgccaagttgtaccaattgccacaaagtacatctgaaatcagtagaatcgctagaaaaggttcaggttccgcatgtagatctttatttggtggttacgtcgcatgggaaatgggtaaagccgaagacggtcatgattcaatggccgtacaaatagctgactcttcagattggcctcaaatgaaagcttgcgtcttggttgtctcagacatcaaaaaggatgtatccagtacacaaggcatgcaattgactgttgcaacatccgaattgtttaaagaaagaatcgaacacgtagttccaaaaagattcgaagtcatgagaaaggctatcgtagaaaaggatttcgccaccttcgctaaggaaactatgatggacagtaactctttccatgcaacttgtttggattcatttccacctattttctatatgaacgacacctcaaagagaataatctcctggtgccacactatcaaccaattctacggtgaaacaatcgttgcttacaccttcgatgcaggtcctaatgccgtcttgtattacttagccgaaaacgaatcaaagttgttcgcttttatatataagttgtttggttccgttccaggttgggataaaaagttcactacagaacaattggaagcttttaatcatcaattcgaatcttcaaactttactgccagagaattggacttagaattgcaaaaggatgtagctagagttatcttgacccaagttggttcaggtcctcaagaaactaacgaatccttgatagatgctaagacaggtttgccaaaagaaaaattgtctggtggtggtggttctggtggtggtggttctggtggtggtggtagtgctgaagcatggtataatttgggtaacgcttattacaagcagggtgactaccaaaaggccatcgaatactaccaaaaggctttggaattggaccctaataacgccgaagcttggtacaatttgggtaatgcctactataaacagggtgactatcaaaaagcaattgaatattaccaaaaggccttggaattggacccaaataacgcagaagcctggtacaatttgggtaacgcctactataagcagggtgactatcaaaaggctatcgaagattaccaaaaggcattagaattggatcctaataacttgcaagctgaagcatggaaaaatttgggtaatgcctattataaacagggtgactaccaaaaagctattgaatactatcaaaaagctttagaattagacccaaataacgcctcagcttggtataatttgggtaatgcatactacaaacagggtgactatcagaaggcaattgaatactatcaaaaggcattagaattagatcctaataacgcaaaagcctggtatagacgtggtaatgcctactacaagcagggtgactatcagaaggcgattgaagactaccaaaaggcattggaattggatccaaacaacagatcaagatccgctggtggtggtggttctggtggtggtggttctggtggtggtggtgcttctgcaatggccgatttggaacaaaaggtattggaaatggaagctagtacatatgacggtgtttttatttggaagatctctgatttcccaagaaaaagacaagaagctgttgcaggtagaatccctgctatttttagtccagcattctacacctctagatacggttacaagatgtgtttgagaatatatttgaatggtgacggtactggtagaggtactcatttgtctttgtttttcgtcgtaatgaagggtcctaatgatgctttgttgagatggccttttaatcaaaaggttaccttgatgttgttggatcaaaacaacagagaacacgttatcgacgcttttagacctgatgtcacttccagttctttccaaagaccagttaatgatatgaacattgcttctggttgtcctttgttttgcccagtctcaaagatggaagctaaaaattcctatgttagagatgacgccatcttcattaaggctatcgttgatttgactggtttaggttcagcaggttccgccgctggttctggtgaatttggttccgccgaagcagccgctaaggaagcagccgctaaagcaggtagtgccggttctgcagccggctctggcgaatttggtagtgcctctattaaattgcaatcatccgacggtgaaatcttcgaagttgatgtcgaaatagcaaagcaatctgttaccataaaaactatgttggaagatttgggtatggatgacgaaggtgacgatgatccagttcctttgccaaatgtcaacgctgcaatattgaagaaagttattcaatggtgcacacatcacaaggacgatccacctccacctgaagacgatgaaaataaggaaaagagaactgacgatattccagtatgggaccaagaattcttgaaggttgatcaaggtacattgttcgaattgatcttggccgctaactatttggacatcaagggtttgttagatgtaacatgtaaaaccgttgctaacatgatcaagggtaaaacaccagaagaaatcagaaagacctttaatattaagaatgatttcactgaagaagaagaagcacaagttagaaaggaaaaccaatggtgcggttctggtgctactaacttctctttgttgaagcaagcaggtgacgttgaagaaaatccaggtccaatgactgctgataataactctatgccacatggtgccgtatcttcatacgctaagttggttcaaaaccaaacacctgaagatatcttggaagaattcccagaaatcatccctttgcaacaaagaccaaacactagatccagtgaaacatccaacgatgaaagtggtgaaacctgtttttcaggtcatgacgaagaacaaattaaattgatgaacgaaaactgcatcgtattggattgggatgacaatgcaataggtgccggtactaagaaagtttgtcatttgatggaaaacatagaaaagggtttgttgcacagagctttctccgtttttatattcaatgaacagggtgaattgttattgcaacaaagagcaacagaaaagatcacctttccagatttgtggactaatacatgttgctctcatcctttgtgcattgatgacgaattaggtttgaagggtaaattggatgacaaaattaagggtgctataactgctgcagtcagaaaattagatcatgaattgggtataccagaagacgaaaccaagactcgtggtaaattccatttcttaaacagaatccactatatggctccatctaacgaaccttggggtgaacatgaaatcgattacatcttattttacaagattaatgcaaaggaaaacttgacagttaacccaaacgttaatgaagtcagagatttcaaatgggtttctcctaatgatttgaagaccatgtttgctgacccatcatataagtttactccttggttcaagatcatctgtgaaaactacttgtttaactggtgggaacaattagatgacttgtctgaagttgaaaacgatagacaaatccatagaatgttgaaattgtctggtggtggtggttctggtggtggtggttctggtggtggtggtagtgccgaagcttggtacaatttgggtaacgcttactacaagcagggtgactaccaaaaggcaatcgaatactaccaaaaggccttggaattggacccaaataacgcagaagcctggtataatttgggtaatgcatattataaacagggtgactatcaaaaggctattgaatattaccaaaaggcattggaattggaccctaataacgctgaagcatggtataatttgggtaacgcctattataagcagggtgactatcaaaaagccatcgaagactaccaaaaggctttggaattggatccaaacaacttacaagccgaagcttggaagaatttgggtaacgcttattacaaacagggtgactaccaaaaagctattgaatactatcaaaaagccttagaattagaccctaataacgcatctgcctggtacaatttgggtaatgcctattacaagcagggtgactatcagaaggctattgaatactaccaaaaagcattggaattggatccaaataacgctaaggcatggtacagacgtggtaatgcctattacaagcagggtgactatcaaaaggcgattgaagattatcaaaaagctttggaattggatcctaacaacagatctagatcagctggtggtggtggttctggtggtggtggttctggtggtggtggtgcttcttcatattaccatcaccatcaccatcacttagaatccacaagtttgtacaaaaaggctggttctggttcaaacaccgttaaggaattaaagaactacatccaagaattggaagaaagaaacgcagaattgaaaaatttgaaggaacatttgaagtttgccaaggctgaattagaattcgaattggccgctcacaaatttgaaggttccgctggtagtgcagccggttccggtgaattcggtagtgcagaagctgcagccaaagaagctgcagccaaggctggttctgcaggttcagctgcaggttctggtgaatttggttccagttactatcaccatcaccatcatcacttagaatccacaagtttgtataaaaaggccggttctggttcacaaaaagtcgcacaattaaagaatagagtagcctacaagttgaaggaaaacgctaagttggaaaacattgtcgcaagattagaaaacgataatgccaacttggaaaaagacatcgctaatttggaaaaggatattgcaaacttggaaagagatgttgccagaggttctggtgctactaacttctctttgttgaagcaagcaggtgacgttgaagaaaatccaggtccaatggaagctaagatagatgaattgataaataacgacccagtttggtcttcacaaaacgaatccttgatcagtaagccatacaaccatatcttgttaaaacctggtaaaaatttcagattaaatttgatcgtacaaatcaacagagttatgaatttgcctaaggatcaattggctatcgtttctcaaatagtcgaattgttgcataactccagtttgttgatcgatgacatcgaagataacgcaccattgagaagaggtcaaactacatcccacttaatttggggtgtccctagtactattaataccgcaaactacatgtacttcagagccatgcaattggtatcacaattgaccactaaggaaccattgtaccattggttgatcacaatttttaacgaagaattgattaatttgcacagaggtcaaggtttggatatctattggagagacttcttaccagaaattatacctacccaagaaatgtacttgaacatggtaatgaataagactggtggtttgtttagattgaccttgagattaatggaagctttgtctccatcttcacatcacggtcattcattggttcctttcataaacttgttgggtatcatctatcaaatcagagatgactacttgaatttgaaggatttccaaatgtccagtgaaaagggtttcgcagaagacataactgagggtaaattgtcattcccaatcgtccatgccttaaacttcacaaaaaccaagggtcaaaccgaacaacacaatgaaatcttaagaattttgttattgagaacttctgataaggacataaagttgaagttgatccaaatcttggaattcgataccaactcattggcttacactaagaacttcatcaaccaattggttaacatgattaagaatgataacgaaaataagtacttgccagatttggcctcccatagtgacactgctacaaatttgcacgatgaattgttgtacatcatcgaccatttgtccgaattgaaattatctggtggtggtggttctggtggtggtggttctggtggtggtggtagtgcagaagcctggtacaacttgggtaacgcttactacaagcagggtgactaccaaaaggctatcgaatactaccaaaaggcattggaattagacccaaataacgctgaagcatggtacaacttaggcaacgcatattataaacagggtgactatcaaaaggccatagaatactaccaaaaggctttggaattggaccctaataacgccgaagcttggtacaacttgggtaatgcttattacaagcagggtgactatcaaaaagcaattgaagactaccaaaaagccttggaattagatccaaataacttgcaagcagaagcctggaagaacttaggcaacgcatactataaacagggtgactaccaaaaagccattgaatattatcaaaaagctttggaattagaccctaataacgcttctgcttggtataacttaggcaatgcctattataagcagggtgactatcagaaagctattgaatattatcaaaaggccttggaattggacccaaataacgccaaggcttggtacagacgtggtaacgcatactacaaacagggtgactatcagaaggctatcgaagattatcaaaaagcattagaattagatcctaataacagatctagatcagctggtggtggtggttctggtggtggtggttctggtggtggtggtgcttctttgtgtactatgaaaaagggtccatctggttacggttttaatttgcattctgataagtcaaagcctggtcaattcataagatcagttgatccagactcccctgcagaagccagtggtttgagagctcaagatagaattgtcgaagtaaatggtgtctgcatggaaggtaaacaacacggtgacgttgtttctgctattagagctggtggtgacgaaactaagttattggtagttgacagagaaggttccgccggtagtgctgcaggttctggtgaatttggttcagctgaagccgctgcaaaagaagccgctgcaaaggccggttctgctggttcagccgctggttctggtgaattcggttcttcatccggtgctataatctatacagttgaattgaagagatacggtggtccattaggtattactatatctggtacagaagaaccattcgatcctatcatcatcagttctttgactaagggtggtttagctgaaagaacaggtgcaatccatattggtgacagaatattggctatcaattcatccagtttgaaaggtaaaccattgtcagaagctatccacttattgcaaatggcaggtgaaaccgttactttgaaaatcaaaaagcaaacagatgcacaacctgcctcttcaggttctggt

SEQ ID NO: 205 - DNA, 18864 bp, synthetic construct
  source 1..18864: mol_type = other DNA
atgaatcatttgagagccgaaggaccagcttctgtcttagcaataggtactgccaatccagagaacatcttgttacaagatgaatttcctgactattacttcagagttaccaaatccgagcatatgacgcagttgaaggaaaagtttagaaagatctgtgataagagtatgatcagaaagaggaactgcttcttaaacgaagagcatttgaagcaaaatcctagattagtggaacacgagatgcaaacattggatgctaggcaggacatgttagttgtcgaagttcctaaattgggtaaagatgcatgtgccaaagctattaaggaatggggtcaacccaagtctaagataactcatttgatttttacaagtgctagcactacagatatgcctggtgcagactatcactgtgccaaactacttggtttatcgccctctgtgaagagagttatgatgtatcaactaggttgctacggtggtggtactgtacttagaatcgctaaagacattgcagaaaataacaagggtgccagggtcttggctgtatgttgcgatattatggcttgcttgtttagaggtccatcagaatccgatttggagctgttggttggtcaagctattttcggtgacggtgctgcagctgttattgttggtgcagaacctgatgagtcagtcggtgaaagaccaatctttgaattggtttctaccggtcaaacgattttaccaaatagtgaaggtacaataggtggtcatatcagagaagctggtttgatattcgatttgcacaaagacgttcctatgctaatatctaacaacatcgaaaagtgtctgatcgaggcttttacccccatcggtatttccgattggaatagtatattctggatcacgcatccaggtggtaaagcaatcctggataaggttgaagagaagctgcatttgaagtctgataagtttgtcgacagcagacatgtattgtcggaacacggtaacatgtcttcatccacagtgctgttcgttatggatgaacttagaaagagatctttggaagagggtaaaagcaccacgggtgacggttttgaatggggtgttctttttggattcggccccggtttgaccgtcgaaagagtagttgttagatctgtaccaattaaatacaagttgtctggtggtggtggttctggtggtggtggttctggtggtggtggtagtgcagaagcctggtacaatttgggtaacgcttactacaagcagggtgactaccagaaggctatcgagtattaccaaaaagcacttgaactggatccaaataacgctgaggcatggtataatttgggcaacgcatattacaaacagggtgactatcaaaaggccatagaatactaccaaaaggctttggagctggatcctaataacgccgaagcttggtacaatttgggaaatgcctattataagcagggtgactatcagaaggcaatagaggactaccaaaaagccctagaacttgatccaaataatttgcaggcagaagcctggaagaatttgggtaatgcttactataaacagggtgactatcagaaagctattgaatactaccaaaaagcactggaattggatcctaataacgcttctgcttggtacaatttgggcaacgcttactacaaacagggtgactaccaaaaagctatcgaatattatcaaaaggctctggaactagatccaaataacgccaaggcttggtatagaaggggaaatgcttattataaacagggtgactaccagaaagcaattgaagactaccaaaaagcccttgaactggatcctaataacagatctagaagcgctggtggtggtggttctggtggtggtggttctggtggtggtggtgcttctggtaacaacttagaaacatacgagtggtacaataagtctatttctagagataaggccgaaaagttactacttgacaccggtaaagaaggtgcttttatggttagagattctagaactccaggtacttatacagtctctgtattcacaaaggctatcatctcagaaaacccatgtatcaagcattaccacatcaaggaaaccaacgactctcctaaaagatattacgtggcagaaaagtacgtttttgattcaatcccactgttgattcaatatcatcagtacaatggtggtggtttggtgactagattgaggtatcctgtttgcggtggtagcgcaggttcggctgcaggatcaggcgaatttggttccgccgaggccgctgcaaaagaagccgctgcaaaggctggatctgcaggctcagccgctggttctggagaatttggttctggttctcatccctggtttttcggtaaaattccaagagcaaaggccgaagaaatgttgtctaaacaaagacacgacggtgcatttttgataagggaaagtgagagcgcacctggtgacttttcgttgtctgttaaattcggtaatgatgtccaacatttcaaggtattgagagatggtgctggtaaatactttttgtgggtcgtaaagttcaattccttgaacgaattagtggattaccatagatcaacttccgttagtaggaaccaacagattttcttgagagatatcgaacaagttccacaacagcctacaggttctggagctactaacttctctttgttgaagcaagcaggtgacgttgaagaaaatccaggtccaatggctgtaaagcatttgatcgtgttgaaattcaaggatgaaatcacagaggcacaaaaggaagagtttttcaagacctacgttaatttggtcaacataatcccagctatgaaagatgtatactggggtaaagacgtgacccaaaagaataaggaagagggttatacccatatagtagaagtgacgttcgaatcagttgaaactatccaagattacatcatacaccctgctcatgttggctttggtgacgtctacagatccttctgggaaaagttgctgatcttcgattacactccaagaaagaaattgtctggtggtggtggttctggtggtggtggttctggtggtggtggtagtgcagaagcctggtataatttgggaaacgcttattacaaacagggtgactaccaaaaggccatcgagtattaccaaaaagctcttgaactggacccaaataacgctgaggcatggtataatttgggtaacgcatactataagcaaggtgactaccaaaaggcaattgaatattaccaaaaggccttggagttagaccctaataacgccgaagcttggtacaatttgggtaatgcctactataaacagggtgactatcaaaaggctatagaggactaccagaaagcactagaacttgatcccaataacttgcaagcagaagcctggaagaatttgggtaatgcctattataagcaaggtgactatcaaaaagctattgaatactaccaaaaagctctggaattggaccctaataacgcttctgcttggtataatttgggtaatgcatactacaagcaaggtgactaccagaaggcaatagagtattaccaaaaagccttagaactagacccaaataacgccaaggcttggtacagaaggggtaatgcctactacaagcagggtgactaccaaaaagctattgaggactaccaaaaagcacttgaactggatcctaataacagatctagatcagctggtggtggtggttctggtggtggtggttctggtggtggtggtgcttccggtcaagatagaagtgaagccacattgattaaaagattcaaaggagaaggtgttagatacaaggctaagctgatcggtatcgatgaagtttctgctgctagaggtgacaaattgtgtcaagactctatgatgaagctgaagggcgttgtcgcaggtgccagatctaagggtgaacataagcaaaagatatttttgacgatctcattcggtggtattaaaatcttcgatgaaaagactggtgctttacaacatcaccatgcagtacacgaaatctcttacatcgctaaggatatcacagaccatagagcattcggttacgtttgcggtaaagaaggcaatcatagatttgtcgctattaaaaccgcccaagccgctgaaccagtcatcttggatttgagagacttattccagctaatctatgaactaaagcaaagagaagaattggaaaagaaagctggtagcgcaggatcggcagccggtagcggagaatttggttctgctgaggctgcagccaaagaagctgcagccaaggccggctctgctggttcagctgcaggctctggtgaatttggttctggttctcatatgggttctcaattttgggtaacttctcaaaagactgaagcttccgagagatgtggtttgcaaggctcctatattttaagggtggaagccgagaagcttaccctacttacgctgggtgcacagagtcaaatattggaacccctgttgttctggccatatactttattgagaagatacggtagagataaagttatgttcagtttcgaagctggtagaagatgcccaagcggtcctggaacttttacattccagacatcacaaggcaatgatatctttcaggcagttgaagccgctattcaacagcaaaaagcccagggtaaagtcggacaggctcaagacattctaagattggaacaccatcaccatcatcatggttctggtgctactaacttctctttgttgaagcaagcaggtgacgttgaagaaaatccaggtccaatgggtttgtcttcagtttgtacattctctttccaaacgaactaccatactttgctgaaccctcacaacaacaatcccaaaacttctttgctttgctacagacatccaaaaacccctattaagtatagctacaacaatttcccatcgaaacattgtagtactaagagcttccatttgcaaaataagtgctccgaatctttgtctatcgctaagaactcaattagagctgcaactacaaatcagacggaaccacctgagtcggataatcactctgtagccaccaaaattttgaactttggtaaagcttgttggaagctgcaaagaccatacacaataatagccttcacctcctgtgcttgcggtttgtttggtaaagaactgttgcataacacaaatttgatttcgtggtctttgatgttcaaggcatttttctttttggttgcaatcctttgcatcgcctcttttaccacgactattaatcaaatctatgatttgcacatcgacagaattaataagcccgatttgccactagcttcaggtgaaatctccgttaatactgcatggattatgtcaatcattgtcgccttgttcggtttaatcatcacaattaaaatgaaaggtggtccattgtacatcttcggctactgtttcggtatattcggtggtatagtatattccgttccaccttttagatggaaacaaaaccccagtaccgctttcttactaaatttcttggcacatatcatcacaaacttcaccttctactacgcttctagagctgctttgggtttgccattcgaattaagaccatcttttacatttttgctggcttttatgaaatcgatgggttctgcattggccttgattaaagatgcatctgacgttgaaggtgacacaaaattcggcatcagtaccttggctagcaagtacggttctagaaatttgactttgttttgttcaggtatcgtattgttatcctacgtggcagccattttagccggtatcatttggccacaagcttttaacagtaatgtcatgctacttagccacgcaatattggccttctggctgatcttgcagacgagagattttgctttaactaattatgaccctgaggcaggtagaagattctacgaattcatgtggaagctgtactacgctgaatatttggtttacgtctttattaagttgtctggtggtggtggttctggtggtggtggttctggtggtggtggtagtgctgaagcatggtacaacttaggcaacgcatactacaagcagggtgactaccagaaggcaattgagtattaccaaaaagccttagaactagacccaaacaatgccgaggcttggtataacttgggcaatgcttattacaaacagggtgactatcaaaaggctatagaatattaccaaaaggcacttgagctggaccctaacaatgcagaagcctggtataacttaggcaatgcttattacaagcagggtgactatcagaaggccatcgaggactaccaaaaggctttggaactggatccaaacaatttgcaggctgaagcatggaagaatttgggtaacgcttactataaacagggtgactatcagaaagcaatagaatactaccaaaaagccctagaacttgaccctaacaatgcctctgcttggtacaacttgggtaatgcttactataagcagggtgactaccaaaaagctatcgaatattaccaaaaagcactggaattggacccaaacaatgcaaaggcctggtatagaagaggtaacgcctactacaaacagggtgactaccaaaaggctattgaagattaccaaaaggctctggaactagatcctaacaacagatctagatccgctggtggtggtggttctggtggtggtggttctggtggtggtggtgcttctgcagaatacgttagagctctgttcgatttcaacggtaacgatgaagaggacttgccttttaagaaaggtgacattttgagaatcagggacaaaccagaagagcaatggtggaatgctgaagattctgagggtaaaagaggaatgattcctgttccctatgtcgaaaagtacggctcagcaggttccgctgcaggatctggcgaattcggttcagccgaggccgctgcaaaagaagccgctgcaaaggctggaagtgcaggcagcgccgctggttccggagaatttggtagtttgattaaacatatgagagccgaagctttattcgattttactggtaactccaaacttgaactgaatttcaaggcaggtgacgttattttcttgttgagtagaattaataaggactggttggaaggtactgttagaggtgctactggaatattcccactttcttttgtgaaaatcctgaagggctcaggtgctactaacttctctttgttgaagcaagcaggtgacgttgaagaaaatccaggtccaatgaaatgtagcactttttctttctggttcgtttgcaagatcattttctttttcttttcttttaatatccaaacttcgatcgcaaatccaagagaaaacttcttaaagtgtttctcacaatacattcctaataacgccacgaatttgaagctggtatacactcagaacaacccactgtacatgagcgtgctaaactcgacaatccataatttgagattcacttccgatactacacccaaaccattagtaatcgtgacaccttctcatgtttcacacattcaaggaaccatactatgctctaagaaagtcggtttgcagattagaacaaggtctggtggtcatgatagtgaaggcatgtcctacatcagtcaagttccattcgttatcgtcgatttgagaaacatgaggtctatcaaaatagacgttcactcacagacggcttgggtcgaggcaggtgccactttgggagaagtttactactgggtcaacgaaaagaatgaaaatttgtctcttgctgcaggttactgtccaactgtctgcgctggtggtcattttggtggtggtggttatggacctcttatgagaaactacggtttggccgctgataatatcattgacgcacatttggtaaatgtgcacggtaaagttctagatagaaagtcaatgggtgaagatttgttttgggcattgagaggtggtggtgctgaatcctttggtataatcgtagcttggaaaattagattggttgcagtcccaaagtctacaatgttctcagttaagaaaattatggaaatccatgagctggtaaagttggtgaataagtggcaaaacatcgcttacaagtacgataaggacttgctgctaatgacccatttcatcacgagaaacatcactgataaccagggtaaaaataagacagcaatacacacctacttctcttcagttttcttgggtggtgttgattccttagtggatttgatgaataagagtttccctgaactgggtattaagaaaactgattgtagacaattgagctggatcgacacaatcatattctatagtggtgttgtcaactacgatactgacaacttcaacaaagaaatccttctggatagaagtgccggacaaaatggcgctttcaaaattaagttggactacgttaaaaagcctatacccgagtcagtatttgtgcagatccttgaaaaactgtatgaagaggatattggtgctggaatgtacgcattatatccatacggtggtataatggatgaaatctccgagagtgccataccattccctcatagagctggtatcttgtacgaactgtggtacatatgttcttgggaaaaacaagaggataacgaaaagcacttaaactggatcaggaacatctataacttcatgactccttacgtttctaaaaaccccagattggcttatttgaattacagagatttggacataggtatcaacgatcctaaaaatccaaacaactacacacaagcaagaatttggggtgaaaagtacttcggtaaaaatttcgatagattggttaaagtcaagaccttagttgaccccaacaactttttcagaaacgaacaatctattccacctttgcctagacataggcacggctctggtgctactaacttctctttgttgaagcaagcaggtgacgttgaagaaaatccaggtccaatgaactgtagcactttttctttttggttcgtttgcaagataatatttttctttttgtcctttaatatccaaatcagtatcgccaacccacaggaaaactttttaaagtgtttctctgagtacatccccaacaacccagctaaccctaagtttatatatacacaacatgatcagctgtacatgagcgtattgaactcgaccattcaaaatttgagattcacttctgacactacacctaagcccttggtcatagtaactccttctaatgtctcacatatacaagcttctatcttgtgctctaagaaagttggtttgcagattagaacaaggtctggtggtcacgatgcagaaggtttatcctatattagtcaagtcccatttgccatagtagatttgagaaatatgcatactgtgaaagttgacatacactcacagactgcttgggtggaagcaggtgccacattgggagaggtttactactggatcaacgagatgaacgaaaactttagtttcccaggtggttactgtcccacagtcggtgttggtggtcatttttctggtggtggttatggagctttaatgagaaactacggtttggctgcagataatatcattgacgcacatttggtgaacgttgatggtaaagttcttgacagaaaatcaatgggtgaagatttgttttgggctatcagaggtggtggtggtgaaaatttcggtataatcgccgcttgcaaaattaagttggttgtcgtacctagcaaagctactattttctctgtcaaaaagaacatggaaatccatggtttagtaaagttgtttaataagtggcaaaacatcgcatacaagtacgataaggatttgatgcttaccacgcatttcagaactaggaacatcacagataaccatggtaaaaataagactacagttcacggatacttctcttcaattttcttgggtggtgttgattctcttgttgatttgatgaataagtcattcccagaactgggtattaaaaagacagattgtaaggaactgagctggatcgacaccacgattttctatagtggtgtggttaattacaacaccgccaacttcaaaaaggaaatcttgctggatagatccgctggtaaaaagaccgctttttctattaaacttgactacgttaagaaactgatccctgaaactgcaatggttaagatattggagaagctgtacgaagaggaagtcggcgtaggcatgtacgttttgtatccatacggtggtataatggatgagatctccgaaagtgccataccatttcctcatagagctggtatcatgtatgaattatggtacaccgctacgtgggagaagcaagaagataacgagaaacacataaactgggtcagatctgtatacaacttcactacaccttacgtttctcagaacccaagattggcatatttgaactacagagatttggacttgggtaaaaccaaccccgaatctccaaataactatacgcaagcaagaatttggggtgaaaagtacttcggtaaaaatttcaacagattggtgaaggttaagacaaaagccgatccaaacaacttctttagaaacgaacaatctattccaccattgccaccaagacatcatggttccggcgctactaacttctctttgttgaagcaagcaggtgacgttgaagaaaatccaggtccaatgtcagaagagtccttatttgaatcttcaccacaaaagatggagtacgaaatcactaactactctgagagacatacagaattgcctggacacttcatcggtttgaacacagttgacaagctggaagagtctccattgagagatttcgtcaagtcccatggtggtcacaccgtaattagtaagatcttgatagctaacaacggtatcgctgcagtcaaggaaattagatctgttagaaagtgggcatatgaaacctttggtgacgatagaacggtccaattcgtagctatggcaactcctgaagacttggaggccaatgctgaatatatcagaatggccgatcaatacattgaagttccaggtggtacaaataacaataactacgctaatgtcgacttaatagtagatatcgctgaaagagcagacgtggatgccgtttgggctggttggggacatgcttccgaaaaccctttgttacccgaaaaattgtctcagagtaagagaaaagttatttttattggtccacctggaaatgcaatgagatcattaggtgacaagatatccagtactatcgtggcacaatcagccaaagttccatgtattccttggtccggcaccggtgttgacacggtgcatgttgatgaaaagactggtttggtttctgtagatgacgatatctatcagaagggatgttgcacttcacctgaagatggtttgcaaaaggctaagagaatcggtttcccagttatgatcaaggcatcagaaggtggtggtggtaaaggtatcaggcaggtcgaaagagaagaggatttcatcgctctgtaccatcaagccgctaatgaaatacccggttctccaattttcataatgaaactagctggaagggcaagacatttggaagttcagctacttgctgaccaatacggcactaatatttccttgttcggtagagattgcagtgttcaaagaagacatcaaaagattatcgaagaggcaccagtcactatagcaaaagccgaaacatttcacgagatggaaaaggcagctgttagattgggtaaattggtcggatatgtaagtgctggaacagtcgaatatttgtacagccatgacgatggtaaattctactttttggaacttaacccaagattacaagttgagcaccctactacagaaatggtttctggtgttaatttgccagctgcacaactgcagattgctatgggtatccctatgcatagaatcagtgatatcaggactctgtacggtatgaatccacacagcgcttcggagattgacttcgaattcaaaactcaggatgcaactaagaaacaaagaagaccaatcccaaagggtcattgtaccgcttgcagaattacgtccgaagaccccaatgatggttttaaaccatctggtggtactttgcacgaactaaactttagaagctcgtctaatgtctggggttatttctcagtaggcaacaacggtaacatccattctttttcagattcccagttcggtcacatcttcgcatttggagaaaataggcaagcctctagaaagcatatggttgtcgctcttaaagaactgtcaatcagaggtgacttcagaaccacggttgaatacttaattaaactgttggaaactgaagacttcgaagataatacgattactacaggttggttggacgatttgataacccataagatgacggcagaaaaacctgatcccaccttggccgttatctgtggtgccgctacgaaggcctttttagcttctgaagaggctagacataagtacatagaaagcctgcaaaagggtcaggtactatcgaaagacttactacaaacaatgtttcctgtggatttcatccacgaaggtaaaagatacaagtttactgttgctaagtctggcaacgataggtacacgttgttcattaatggtagcaagtgcgacatcattctaagacaactttcagatggtggtttgctgatcgcaattggtggtaaatcacatactatctattggaaggaagaggtcgcagccacaagattgagtgtagacagcatgaccacgttgttagaggttgaaaacgatccaactcaattaagaacaccatctcctggtaaacttgtgaaatttctggttgaaaatggcgagcatataatcaagggtcaaccctacgctgagattgaagttatgaaaatgcagatgccattggtttctcaagaaaacggtatagttcaactacttaaacagcctggatcaaccatagtagctggtgacatcatggcaattatgacgttagacgatccatccaaggtgaaacatgctcttccttttgagggtatgctgcccgatttcggttctccagttattgaaggcactaaaccagcatacaagtttaaatcgttggtttctacactggaaaacatcctaaagggttacgataaccaagttattatgaatgcttctttgcaacagttgatagaagtcttgagaaatcctaagttaccctattcagaatggaaattgcatattagcgctcttcactccagattgcctgcaaaattggatgaacaaatggaagagctagtcgctagatctttgagaagaggtgctgtatttccagcaaggcaattgagtaagctaattgacatggcagttaaaaacccagaatacaaccctgataaactgttgggtgccgtagtggaaccattggcagatattgcccataagtactctaatggtttagaagctcatgagcactcaatcttcgtgcatttcttggaagagtactacgaggttgaaaaattgttcaacggtcctaacgtcagagaagagaacatcatcctgaagttgagagatgaaaacccaaaggacttggataaagtcgctcttactgtactgagtcatagcaaggtttctgccaaaaataacttaatcctagctatcctgaagcactaccaacctttgtgtaagctgtcatccaaagtttctgcaatattttcaactccattgcaacatatcgtagagcttgaatctaaggctaccgcaaaagtggctttgcaggcaagagaaattttgatccaaggtgctttgccatcagttaaagaaagaacagagcaaatagaacacatcctgaagagtagcgttgtcaaagtcgcatacggttcgtctaatcctaagagatctgaacccgatttgaatatacttaaggatttgatcgattcaaattacgtagtgtttgacgttttactacagttcttaactcatcaagatcctgttgtcacagctgcagccgctcaagtctatataagaagggcctatagagcttacactatcggtgacattagggtacacgaaggcgtgacagttccaatcgtggaatggaaatttcaattgccctccgcagcctttagtaccttcccaacggtaaagtcaaaaatgggtatgaacagagctgtttctgtttctgatttgagctatgtggctaattcgcaatcatcccctttaagagaaggtattctaatggctgtggaccatttggacgatgttgatgaaattttgtctcaatctttggaagttattccaagacaccaaagtagctcgaatggtcccgctccagataggtctggatcttcagcaagtttaagcaacgtagccaatgtgtgtgttgcttccactgagggttttgaaagtgaagaggaaatcttggttagattgagagaaattttggatttgaacaaacaagaattgattaatgcttccatcagaaggatcacattcatgttcggttttaaagatggtagttaccctaagtactacacctttaatggtcccaactacaacgagaacgaaactatcagacatatcgaacctgccttagctttccaattggaactgggtagattgtcaaacttcaacatcaagccaattttcactgataacagaaacatccatgtgtacgaagctgtttcaaagacatccccattagataagagatttttcaccagaggcatcattaggacgggtcacattagagatgatattagcatacaagagtacttgacttcggaagctaacagattaatgtctgacatcctagataatttggaagttaccgacacgtcgaactctgatttgaaccatatctttattaacttcatcgcagtgttcgacatatctcctgaggatgttgaagctgcatttggtggtttcttggaaagattcggtaaaagattgctgagattgagagtctccagtgctgaaatcagaatcatcattaaggatccacaaactggtgcccctgtacccctgagagctttgatcaataatgtttctggttacgtaattaaaaccgagatgtacacggaagtcaagaatgctaagggtgaatgggtattcaagagcttgggtaaacccggctcgatgcacttaagaccaattgcaacaccatatcctgtcaaagaatggttgcaacctaagagatacaaagcccacttaatgggtactacatacgtttacgatttcccagaattgttcagacaggcttcttcttctcaatggaagaatttttccgccgacgttaagctgactgacgatttctttatcagtaacgaactaatcgaggatgaaaatggtgaacttacagaggttgaaagagagccaggagcaaatgccattggcatggtcgcttttaagatcactgtaaagacaccagaatatcctaggggtagacaattcgtagtggttgcaaacgacatcacctttaaaattggttctttcggacctcaagaagatgagtttttcaataaggttactgaatacgctaggaaaagaggtataccaagaatctacttggccgctaattctggagcaaggattggcatggccgaggaaatagtgcctttatttcaggttgcatggaacgacgcagccaacccagataagggattccaatatttgtatttgacttctgagggtatggaaacattgaaaaagttcgataaggaaaactcagtgctgaccgagagaactgttattaatggagaggaaaggttcgtaatcaaaactataatcggttctgaagatggtttgggcgtggagtgtctgagaggtagcggtttgattgctggtgcaacttctagagcttaccatgatatttttactatcacactggtcacttgcagatctgtaggcataggtgcttatttggttagattgggtcaaagggccatccaggtcgaaggccaacctattatattgactggtgcccccgctataaacaaaatgctgggtagagaagtttatacctccaatttgcagttgggtggtacgcaaatcatgtacaataacggtgtttctcatttgacagctgtagacgatttggctggtgtggaaaagattgttgaatggatgtcatatgtgccagctaaaagaaacatgcccgttccaatattggaaactaaggacacatgggatagaccagtagattttacccctacgaatgacgaaacctatgatgtgagatggatgattgagggtagggaaactgagtctggttttgaatacggtttgttcgataagggttctttctttgaaacattatcaggctgggccaagggtgtcgtagtgggaagagctagattgggtggtattcctctaggtgttattggtgtagaaactagaacagttgaaaatttgatccccgcagatccagccaaccctaattctgctgaaactttaattcaggaacctggtcaagtttggcatcccaactcagcttttaaaaccgcacaggccattaatgatttcaacaacggtgaacaattgccaatgatgatactggctaactggagaggtttttctggtggtcaaagggatatgttcaacgaagttttgaagtacggtagttttatcgtcgacgcactggtagattacaagcaacctatcataatatacattccaccaactggtgaattaagaggtggttcttgggttgtcgtagacccaaccattaacgcagatcagatggaaatgtacgccgatgtgaatgctagagcaggtgttttggaaccacaaggaatggttggtattaagtttagaagagaaaaattgctggatactatgaacagattagacgataagtacagggaattgagatctcaactgagcaataagtctttggctccagaagttcatcaacagatctctaagcaactggctgatagggaaagagaattgttgccaatatacggtcagatctcattgcaatttgccgacttacacgataggtcatccagaatggtggctaagggtgttatttcaaaagaattagagtggacagaagctagaagatttttcttttggagattgagaagaagattgaacgaggaatatttgattaaaagattgtcacatcaagttggcgaggcttctagattggaaaagatcgcaaggattagatcttggtatccagcatcagtcgatcacgaagacgatagacaagtagccacttggattgaggaaaattacaagacactggacgataagttgaagggtttaaagctagaatcctttgcccaagacttggctaaaaagattagaagtgaccatgataatgctatcgatggtttgagtgaagttattaaaatgcttagcactgacgataaggaaaaactgttgaagacattgaagaaactgtctggtggtggtggttctggtggtggtggttctggtggtggtggtagtgccgaagcttggtataacttgggaaatgcttattacaagcagggtgactaccaaaaggccatagaatactaccaaaaggctcttgagctggatcctaataacgcagaagcctggtataacttaggcaatgcatactataaacaaggtgactaccaaaaggcaatagagtactaccaaaaggccttggaattagatccaaataacgctgaggcatggtataacttgggcaacgcctactataaacagggtgactatcaaaaggctatagaagattaccagaaggcactagagcttgatcctaataacttgcaagccgaagcttggaagaacttaggaaatgcatactataagcaaggtgactatcaaaaagctattgaatattaccaaaaggctctggagttggatccaaataacgcatctgcttggtacaacttaggcaacgcctactataagcagggtgactatcaaaaagcaattgaatattatcaaaaggccttagagctagatcctaataacgctaaagcatggtataggagaggcaatgcatactacaaacagggtgactaccaaaaagctatagaagattaccaaaaggcacttgaactggatccaaataacagatctagatccgctggtggtggtggttctggtggtggtggttctggtggtggtggtgcttctggttctcatatgagattgggagcccaatctattcagccaaccgctaacttagatagaacggacgatttggtctatttgaatgtaatggaattggttagagctgttttggagttgaaaaatgaactagcacaattgccaccagaaggttacgtggttgtcgtaaagaatgttggtttgactcttagaaagttgataggctcggtcgacgatttgctaccatctttgccatcttcttctagaactgaaatagagggtacacaaaagcttctgaacaaagatttggctgaattgattaataagatgagattggcacaacagaacgccgttacttctttgtctgaggagtgtaagagacaaatgctaactgcttctcatactttggctgttgatgcaaagaacttgttagacgctgtggatcaagcaaaagttttagccaatttggctcacccacctgccgaaggttctgctggatcagctgcaggatccggcgaatttggttctgctgaagccgctgcaaaagaggctgctgcaaaagctggatctgcaggtagtgctgctggtagcggagaatttggttctggtgccatggctactcctggttcagaaaacgttctaccaagagaaccattgattgcaacagccgtgaagttcttgcagaactctagagttagacaatctccattggcaactagaagagcatttttgaaaaagaaaggtttgaccgacgaggaaattgatatggctttccaacagtccggtactgcagccgatgaaccatcttcattgtggggaagtggc

SEQ ID NO: 206 - DNA, 19473 bp, synthetic construct
  source 1..19473: mol_type = other DNA
atgggttctgctggttcagctgcaggttctggtgaattcggttccgctggtagtgccgctggttctggtgaatttggttctgctggttcagcagccggttctggtgaattctcctattaccatcaccatcaccatcacttggaatctacttcattatacaaaaaggctggttccggtagtgccagaaacgcttacttgagaaagaaaattgctagattgaagaaagataatttgcaattggaaagagatgaacaaaacttggaaaagattatcgctaatttgagagatgaaatagcaagattggaaaatgaagttgcttctcatgaacaaggttccgcaggtagtgccgccggttctggtgaatttgctgaagccgctgcaaaggaagccgctgcaaaagcaggttctgccggttcagccgctggtagtggtgaattttcttactatcaccatcaccatcatcacttggaatctacctcattatataaaaaggccggttccggtagtaacttggttgctcaattagaaaatgaagtcgcatcattggaaaacgaaaacgaaactttgaaaaagaaaaacttacataagaaagatttgatcgcttacttagaaaaggaaatagcaaatttgagaaagaaaatagaagaaggttccgctggtagtgcagccggtagtggtgaattcggttctgctgaagctgcagccaaggaagctgcagccaaagaagccgctgctaaagaagctgcagccaaagctggttctgcaggttctgccgcaggttccggtgaatttggttcttcatactatcaccatcaccaccaccacttggaatctacctcattatacaagaaagctggttccggtagtcaaaaggtcgctgaattgaaaaacagagtagctgttaagttgaacagaaacgaacaattgaaaaataaggtagaagaattgaaaaatagaaacgcttacttgaaaaacgaattggcaactttggaaaacgaagtagctagattagaaaacgatgttgctgaaggttctgctggttctgctgctggttctggtgaattcgctgaagcagccgctaaggaagcagccgctaaagccggttccgccggttctgctgcgggctctggtgaattttcctactatcaccatcatcatcaccacttggaatctacatcattatataagaaagccggttccggtagtaatgaagttactacattggaaaacgatgctgcttttattgaaaacgaaaacgcatacttagaaaaggaaatcgctagattgagaaaggaaaaggccgctttgagaaatagattagctcataagaaaggttctgctggtagcgctgctggctctggtgaatttggttccgccgaagccgctgctaaggaagccgctgccaaagaagccgctgccaaggaagccgctgctaaggctggttccgccggttcagctgcaggctctggtgaattcggttctagaccacctaccatctctaatccacctccattgatttccagtgctaaacatccatccgtcggtagtgcaggttccgctgccggctctggcgaatttgccgaagctgctgccaaagaagcagccgctaaagctggttcagcaggttccgctgccggatctggcgaattcaatttcttgcaatctagaccagaacctactgctcctccagaagaaagtttcagatctggtggttcagctggttccgccgcaggatctggcgaatttggttccgcagaagctgccgctaaagaagctgctgcaaaagaagcagccgccaaagaagctgctgcaaaagccggtagtgctggttcagctgccggttccggtgaattcggttcttcaaaaggtaccggtttaaatccaaacgctaaagtttggcaagaaattgctcctggtaacggttctgcaggttccgcagctggttccggtgaattcgccgaggccgctgctaaggaagcagcagccaaagcaggtagtgctggttccgcagctggttcaggtgaattcccagacggtggtaccactttcgaacatttgtggtccagtttagaacctgattctacatacggttctgccggttctgcagcaggcagcggtgaattcggttctgccgaagctgctgctaaagaagctgctgccaaggaagctgctgctaaggaagctgctgccaaagccggtagtgcaggttctgctgccggttcaggtgaatttggttcttcttactatcaccaccaccaccatcacttggaatctacatcattatacaagaaagccggttctggtagtaagagaatcgcatacttaagaaagaaaatcgctgcattgaagaaagataacgcaaacttagaaaaggacatcgctaacttggaaaacgaaatcgaaagattgattaaagaaatcaaaaccttggaaaatgaagttgcatctcatgaacaaggttcagccggttctgcagcgggctccggtgaatttgccgaagctgcagcaaaagaagctgccgctaaggctggtagtgctggttctgctgcaggcagcggtgaattttcttactaccaccatcaccaccatcacttggaatctacttcattatataagaaagcaggttctggtagtaacttgttagcaacattaagatctaccgctgcagtcttggaaaacgaaaaccatgtattggaaaaagaaaaggaaaaattgagaaaggaaaaagaacaattgttgaataagttggaagcttacaaaggttcagcaggttctgcagcgggctctggcgaattcggttccgccgaagctgcagcaaaggaagctgcagctaaagaggccgctgcaaaagaagctgctgccaaagcaggtagtgcaggttccgcagccggctccggcgaatttggttcaccagctacatcccaacatcctccacctccacctggtcatagatctcaagctccttcacatggttccgcaggtagtgcagctggatctggcgaattcgccgaagctgccgctaaggaagctgctgcaaaagctggttccgctggttcagcagcaggttccggtgaattcgaattgaattctttgttgatattgttagaagcagccgaatatttggaaagaagagatagaggttctgccggtagtgctgcaggtagcggcgaatttggttctgcagaagcagccgccaaggaagcagctgcaaaagaagcagcagctaaagaagcagctgcaaaagccggttctgctggttcagccgcaggatctggagaattcggttccagaccacctacaatttccaatccacctccattgatctcttctgccaagcatccatccgttggtagtgcaggttcagctgccggtagtggtgaatttgccgaagccgccgctaaggaagccgccgccaaagcaggttcagccggttccgccgcaggttcaggtgaattcaatttcttgcagtcaagaccagaacctaccgctcctccagaggagagtttcagatctggtggtagtgccggttcagctgccggctctggagaatttggttctgcagaggctgctgccaaggaagccgcagctaaagaagccgctgcgaaagaagccgccgctaaagctggtagtgcaggtagtgctgcgggatctggcgaattcggttcttctaagggtactggtttgaaccctaatgccaaggtctggcaagaaatcgcccctggtaacggttccgcaggttccgccgcaggtagtggtgaattcgccgaggctgccgccaaggaagccgccgctaaggcaggtagtgctggttcagcggccggctctggtgaatttccagacggtggtacaacctttgagcatttgtggtccagtttagaacctgattctacgtacggttctgctggttccgctgcaggatctggcgaattcggttccgcggaagccgccgcaaaagaagccgccgccaaagaagccgccgcaaaggaagccgcagcaaaggcaggtagtgccggctccgccgctggcagtggcgaatttggttcttcatattatcaccatcatcatcatcacttggaatctacttcattatacaagaaagcaggttccggttctaaaagaattgcttacttaagaaagaaaatcgcggctttgaagaaagacaatgctaacttagaaaaagatattgccaacttggaaaatgaaatcgaaagattaattaaggaaattaaaacattggaaaacgaagttgcatcacatgaacaaggttcagctggttccgctgcagggtccggcgaatttgcagaagccgccgccaaggaagccgcagccaaagctggtagtgcaggttctgccgctggctctggcgaattttcttactatcatcatcaccatcaccacttggaatctacttcattatacaagaaagcgggttcaggttctaacttgttagcaactttaagatctacagccgctgttttagaaaatgaaaaccatgtcttagaaaaagaaaaggaaaagttgagaaaggaaaaggaacaattattaaataagttagaagcctacaagggttcagcaggttccgcagcaggctcaggcgaatttggttctgcagaagcggctgctaaggaagctgccgcaaaggaagcagctgctaaggaggccgctgcaaaggctggttctgctggttccgccgcgggctctggagaattcggttccgctttggttgatgacgccgctgattatgaacctccaccttcaaataacgaagaagctttaggttccgctggttccgctgcaggttccggcgagttcgcagaagccgcagcaaaagaagccgcagctaaggcaggtagtgccggatccgccgctggcagtggagaattcagagaattgttcgatgacccatcttacgtcaacgtacaaaatttggataaagctagacaaggttccgccggttctgcagcgggatctggggaatttggttctgcagaagctgccgccaaagaagctgcagctaaagaagccgcagccaaagaagctgctgctaaggccggttctgctggttctgccgcaggatctggggaattcggttccaagaatactaagagtatgaacttcgataacccagtttacagaaagactacagaagaagaaggttcagccggttcagccgccggttccggtgaatttgcagaggctgccgctaaagaggctgccgctaaggccggtagtgctggttctgcagccggctccggagaattcagatctttgccatccacatggattgaaaacaaattatacggcatgtcagaccctaattggggttctgcaggttcagctgcgggatctggtgaattcggttcagcagaagccgcagccaaggaagccgctgcaaaggaggccgctgccaaagaagcagctgctaaggctggttcagccggttccgcagccggcagtggtgaatttggtagtgttgtcgataattctccacctccagctttgcctccaaagaaaagacaatctgctccatctggttcagcaggttcagccgctggttcaggtgaatttgccgaagcagctgccaaggaagctgccgccaaggcgggcagtgcaggttcggctgcggggtctggtgaattcactcaaagatctaaaccacaacctgcagttcctccaagaccatctgctgacttgattttaggttccgccggttccgcagctggctctggcgaattcggttccgctgaggctgccgctaaagaagcggccgctaaagaggcagccgctaaagaggcggccgctaaagcaggttctgcaggttcagcagcaggtagtggtgaatttggttctacagatgaagaaagagaagaaaccgaagaagaagtttatttgttgaactctaccactttgggttcagctggttctgctgcgggttctggcgaatttgcagaagcagctgctaaggaagccgcggcaaaggctggttctgcgggctccgccgcaggttctggtgaatttgatggtaatgtatctggtactcaaagattagactcagctaccgttagaacttattcatgcggttctgccggtagtgcagcgggctctggggaattcggttccgcagaagccgctgcaaaagaagccgctgcaaaagaagccgctgcgaaggaggctgctgctaaggcaggttccgccggtagtgctgcgggttccggcgaatttggttccagttactatcaccatcatcaccaccacttggaatccacaagtttatataagaaagctggttctggttcacaaaaggtagctcaattgaaaaatagagttgcatacaagttgaaggaaaacgctaagttggaaaacatagtagcaagattagaaaacgataacgctaatttggaaaaggacatcgcaaatttggaaaaggatatagctaacttggaaagagatgttgctagaggttctgctggtagtgccgcaggctctggcgaattcgctgaagctgccgctaaagaggctgcggctaaagctggttcagctggttctgcagcggggtctggtgaattttcttattatcaccatcatcaccatcacttggaatccaccagtttatacaagaaagccggctctggttcaaacactgttaaggaattgaaaaattacattcaagaattggaagaaagaaacgctgaattgaaaaatttgaaggaacatttgaagtttgcaaaagccgaattggaattcgaattagcagcccataaatttgaaggttctgccggttctgccgccggatctggagaatttggttctgcggaggctgccgctaaagaagccgccgctaaagaggctgcagctaaggaagctgcagcaaaggctggttctgccggttccgctgccggctccggcgaatttggttcacatgatgactccttgccacatcctcaacaagctacagatgactctggtcatgaatccgacggttccgcaggctctgctgccggctccggcgagtttgctgaagccgctgctaaagaggctgctgctaaagccggttctgccggttcagcagctggatctggagaatttggttccccaaatgctggtagtgttgaacaaaccccaaagaaacctggtttgagaagaagaggtagtgctggttctgccgctggctccggagaatttggttcagccgaagctgcggccaaagaggctgctgcaaaggaggctgcggctaaggaagccgccgctaaagccggttcagctggttccgcggcaggctccggggaatttggttcttcttattatcaccaccaccaccatcacttggaatccacaagtttatacaagaaagcaggctctggttcattcgaaaacgtcactcatgaattcattttggcaaccttagaaaacgaaaacgctaagttgagaagattagaagcaaagttggaaagagaattggctagattaagaaatgaagtagcttggttgggttctgcgggctcggccgctggctctggtgaattcgccgaagctgcggccaaggaggctgccgcaaaggccggttctgccggttccgcagcgggatccggcgaattttcttactaccatcatcaccatcaccacttggaatccacaagtttatacaagaaagcgggttctggttcacaaaaagttgaagaattgaaaaataagatagcagaattggaaaacagaaacgctgtaaagaaaaatagagttgcacatttgaagcaagaaatcgcttacttgaaggatgaattagcagcccatgaattcgaaggtagtgccggttccgctgctggctcaggcgaatttggtagtgcagaagctgccgctaaggaggctgccgccaaagaagcagccgcaaaagaagctgccgcaaaagccggttctgcgggctctgctgccggatccggcgaattcggttcagtctccagtactaaattagtatcctttcatgatgacagtgatgaagacttgttacatatcggttctgcaggctcagccgctggctctggagagtttgcagaggcagctgctaaagaagccgccgcaaaggcaggttctgcaggttctgcagctggtagtggtgaattcgctgctgcaaccccaatatctacttttcatgatgactcagacgaagacttgttgcatgtcggttccgcaggttcagcagcgggatccggtgaatttggttcagcagaagctgccgccaaggaggccgctgctaaagaagcagcagccaaggaagcagcagcaaaggccggctctgctggttctgctgccgggtccggcgaatttggttcttcttattaccaccatcatcatcaccacttggaatccacaagtttatataagaaagccggttctggttcacaaaaggtggaatcattaaaacaaaagattgaagaattgaagcaaagaaaagcacaattgaaaaatgatattgccaatttggaaaaggaaatcgcttacgcagaaacaggtagtgccggttcagccgcgggctctggtgaattcgcagaagctgccgcaaaagaagctgcagcaaaagccggttctgcaggctctgctgctggctctggcgaattttcctactatcatcatcatcatcatcacttggaatccacaagtttatacaagaaagcgggtagtgaatttttcagaagagaaagaaacaagatggcagccgctaagtgtagaaacagaagaagagaattgactgatacattacaagctgaaacagatcaattagaagacgaaaaatcagctttgcaaaccgaaatcgcaaatttgttgaaagaaaaagaaaaattggaattcattttagcagcccatagaccagcttgcaaaatacctgatgacttgggttttccagaagaaatgtctttagaaggtagtgccggtagtgccgctggctcaggtgaatttggtagtgcagaagctgccgcgaaagaagccgcagctaaagaagctgccgccaaagaggcagccgcaaaggcaggttcagcaggttcagctgccgggtccggggaatttggttcattccaaatgccagctgacactcctccacctgcatatttgccacctgaagatcctatgacaggtagtgccggttctgctgccgggtctggcgaattcgctgaagccgctgctaaggaggctgcagctaaggccggctctgcaggttccgctgcaggttcaggtgaatttgaaagagaatctaacgaagaaccacctccaccttatgaagatccatactggggtaatggtggttctgccggtagtgccgccggctcaggcgaatttggttctgcggaggctgctgcaaaggaagctgcggccaaggaagctgccgcaaaagaggctgctgccaaggccggttcagcaggttcagcagctgggtccggtgaatttggttccagttattatcaccaccatcatcaccacttggaatctacctcattatataagaaagcgggttccggtagtcaaaaagttgcagaattgaaaaacagagttgctgtcaaattaaatagaaatgagcagttgaaaaataaggtcgaggagttgaaaaatagaaacgcatacttgaaaaatgaattggctactttggaaaacgaagtcgcaagattagaaaatgatgtagctgaaggctctgctggttccgcagcgggctcaggtgaattcgccgaagcagccgcaaaggaagctgccgctaaggccggctcagcaggttctgccgccggaagcggtgaattttcttattaccaccaccaccatcaccacttggaatctacttcattatacaagaaagcggggtccggtagtaacgaagtcacaaccttagaaaatgatgcagcctttatagaaaacgaaaatgcctacttagaaaaagaaattgcaagattgagaaaggaaaaagctgcattgagaaacagattagcccacaagaaatcttactatcaccaccatcatcatcacttggaatctacatcattatacaagaaagcgggctccggtagtgctagaaatgcctacttaagaaagaaaatagccagattgaagaaagacaatttgcaattagagagagatgaacagaacttagaaaagattatagccaatttgagagatgaaattgctagattagaaaatgaagtagcttctcatgaacaaggtagtgctggctccgctgccggctccggagaatttgccgaagctgccgccaaggaagccgcggccaaggctggttccgctggttctgctgccggatctggagaattttcctattaccatcatcatcatcatcatttggaatctacatcattatacaagaaagcgggatctggttctaacttggtcgcccaattggagaacgaagtcgcatcattggagaacgaaaacgaaaccttgaagaaaaagaacttacacaaaaaggatttgatagcttacttagaaaaagaaatcgctaatttgagaaagaaaattgaagaaggtagtgcaggttcagccgctggctccggtgaatttggttcagcggaggctgccgctaaggaggcagccgctaaagaagcagccgctaaggaggctgcagcaaaagcaggttccgcaggttctgcagcgggttccggagaatttggttctgaacaaaagttgatctctgaagaagatttggaacaaaagttgatatctgaagaagacttggaacaaaaattaatatcagaagaagatttgggtagtgcaggttcagcagctggttctggagaatttggttcagcaggttctgccgctggaagtggcgaattcggtagtgccggctccgctgctggctctggcgaatttggttctggtgctactaacttctctttgttgaagcaagcaggtgacgttgaagaaaatccaggtccaatgggttctgctggttcagctgcaggttctggtgaatttggttccgcaggtagtgccgctggttctggtgaattcggttctgctggttcagcagccggttctggtgaattttcatattaccatcaccatcaccatcacttggaatccaccagtttatacaaaaaggctggttctggttcagctagaaacgcatatttgagaaagaaaattgctagattgaagaaagataacttgcaattggaaagagatgaacaaaatttggaaaagattatcgccaacttaagagatgaaatagcaagattggaaaacgaagtagcttctcatgaacaaggttccgcaggtagtgcagctggttctggtgaatttgctgaagccgctgcaaaggaagccgctgcaaaagctggttccgctggttcagccgctggttccggtgaattcagttactatcaccatcaccatcatcacttggaatccacaagtttatataaaaaggccggttctggttcaaatttggttgctcaattagaaaacgaagtcgcatctttagaaaacgaaaacgaaacattgaaaaagaaaaatttgcataagaaagatttgatcgcttatttggaaaaggaaatcgcaaacttgagaaagaaaatagaagaaggttccgctggttctgctgctggttccggtgaatttggttcagctgaagctgcagccaaggaagctgcagccaaagaagccgctgctaaagaagctgcagccaaagcaggttctgccggttctgccgcaggttccggtgaattcggttcttcagctactagagaattggatgaattgatggcatccttaagtgacttcaagatacaaggtggttccgctggttctgcagccggctctggcgaattcgcagaagcagccgctaaggaagcagccgctaaagctggttctgcaggttctgctgccggttctggtgaattcgatttggctttgtctgaaaactgggcacaagaattcttggctgcaggtgacgctgttgatggttctgctggtagtgctgccggttcaggtgaatttggtagtgctgaagctgctgccaaagaagcagccgctaaagaagctgctgccaaggaagctgccgctaaagcaggttccgccggttctgccgccggctccggcgaatttggttcagattataaggatgacgatgacaaggattacaaagacgatgatgacaaggattataaagatgacgatgacaaaggttccgctggtagtgccgccggctctggagaattcggttctgccggttcagctgccggctccggagaatttggttccgctggtagtgcagccggttcaggtgaattcggttctggtgctactaacttctctttgttgaagcaagcaggtgacgttgaagaaaatccaggtccaatgagtgctaaggcaatttctgaacaaactggtaaagaattgttgtacaagtttatttgtactacatcagccatccaaaatagattcaaatacgctagagttaccccagatactgactgggctagattgttacaagatcatccatggttgttatctcaaaacttggttgtcaaacctgaccaattaattaagagaagaggtaaattgggtttagtaggtgttaatttgacattggatggtgtaaagtcttggttgaaaccaagattaggtcaagaagccacagttggtaaagctaccggtttcttgaaaaatttcttgatcgaaccatttgtccctcattcacaagccgaagaattctatgtatgtatctacgctactagagagggtgactatgttttatttcatcacgaaggtggtgtcgacgtaggtgacgttgacgccaaggctcaaaagttgttggttggtgtcgatgaaaagttgaacccagaagacattaaaaagcatttgttggttcacgcacctgaagataaaaaggaaatattggcctcctttataagtggtttgtttaatttctacgaagatttgtacttcacctacttggaaattaacccattagtagttactaaggatggtgtatatgttttggacttagctgcaaaagttgatgcaacagccgactacatttgtaaggtcaaatggggtgacatcgaatttccacctccattcggtagagaagcttatccagaagaagcctacattgctgatttggacgctaagtctggtgcatcattgaagttgacattgttgaaccctaaaggtagaatttggaccatggttgctggtggtggtgctagtgtcgtatattctgatactatatgcgacttgggtggtgttaacgaattggcaaactacggtgaatactcaggtgccccatccgaacaacaaacatacgattacgctaagaccatcttgtccttaatgactagagaaaagcatcctgatggtaaaatcttgatcatcggtggtagtatcgcaaattttactaacgttgccgctacattcaagggtatcgtcagagctataagagattaccaaggtccattgaaggaacacgaagtaacaatattcgttagaagaggtggtcctaactaccaagaaggtttgagagtcatgggtgaagtaggtaaaaccactggtataccaatccatgtctttggtacagaaacccacatgactgcaatagttggtatggccttaggtcatagaccaatccctaatcaacctccaaccgcagcccacactgcaaatttcttgttaaacgcctctggttcaacttccacaccagctccttctagaacagcaagtttctctgaatcaagagctgatgaagtcgctccagctaagaaagcaaaaccagccatgcctcaagactccgttccaagtcctagatctttgcagggtaaatctactactttgttttctagacatactaaggctatagtatggggtatgcaaacaagagcagttcaaggcatgttggatttcgactatgtttgtagtagagatgaaccatctgttgctgcaatggtctatccttttactggtgaccataagcaaaaattctactggggtcacaaggaaatattgatcccagtttttaagaacatggccgatgctatgagaaaacatcctgaagtcgacgtattgattaacttcgcctcattaagatccgcttacgattctacaatggaaaccatgaactacgctcaaataagaaccatcgctatcattgcagaaggtattccagaagccttgactagaaagttgattaagaaagctgatcaaaaaggtgtcacaataatcggtccagctaccgtaggtggtattaagcctggttgtttcaagatcggtaacactggtggtatgttggataacatattggcatctaagttgtatagaccaggttcagtcgcttacgtatccagaagtggtggtatgtccaacgaattgaacaacatcatcagtagaactacagatggtgtatacgaaggtgttgctattggtggtgacagatacccaggttctacttttatggatcatgtattgagatatcaagacacacctggtgttaaaatgattgttgtcttgggtgaaataggtggtactgaagaatacaagatatgcagaggtatcaaagaaggtagattgacaaagccaatcgtttgttggtgcattggtacttgtgcaacaatgttttcttcagaagttcaattcggtcatgcaggtgcctgcgctaatcaagcttcagaaacagcagttgccaagaaccaagcattaaaagaagccggtgtttttgtccctagatctttcgatgaattaggtgaaatcattcaatcagtctatgaagacttggtagctaatggtgtaattgttccagcacaagaagttcctccacctactgtccctatggattactcttgggcaagagaattgggtttaattagaaagccagctagttttatgacctctatatgtgatgaaagaggtcaagaattgatctatgctggtatgcctattactgaagtattcaaagaagaaatgggtatcggtggtgttttaggtttgttgtggttccaaaagagattgccaaagtactcttgtcaattcattgaaatgtgcttaatggttacagctgatcatggtcctgctgtctcaggtgcacacaataccataatctgcgctagagctggtaaagatttggtttcttctttgacctcaggtttgttaactattggtgacagatttggtggtgcattagacgccgctgcaaagatgttttcaaaagctttcgattccggtataatcccaatggaattcgttaataagatgaaaaaggagggtaaattgataatgggtatcggtcatcgtgttaagtctatcaataaccctgatatgagagtacaaatcttgaaggactatgttagacaacactttccagccacacctttgttagattacgctttggaagttgaaaagattaccacttctaaaaagccaaatttgatcttgaacgttgatggtttaattggtgttgcttttgtcgacatgttgagaaactgtggttccttcactagagaagaagctgatgaatatatcgacattggtgcattgaatggtatctttgttttaggtagatctatgggtttcattggtcattacttggatcaaaagagattaaagcaaggtttgtacagacatccatgggatgacatttcttacgttttacctgaacacatgtcaatgaaattgtctggtggtggtggttctggtggtggtggttctggtggtggtggtagtgccgaagcttggtacaatttgggtaacgcatactacaagcagggtgactaccaaaaggcaattgaatattaccaaaaggccttggaattagacccaaataacgcagaagcctggtataatttgggtaatgcttattataaacagggtgactatcaaaaggctatcgaatactaccaaaaggcattggaattagaccctaataacgctgaagcatggtataatttgggtaacgcttattataagcagggtgactatcaaaaagccatcgaagactaccaaaaggctttggaattagatccaaataacttacaagccgaagcttggaagaatttgggtaacgcttactataaacagggtgactaccaaaaagcaattgaatactatcaaaaagctttagaattggaccctaataacgcatcagcctggtacaatttgggtaatgcttactataagcagggtgactatcagaaggccattgaatactatcaaaaggctttagaattggatccaaataacgctaaagcatggtacagacgtggtaacgcttattacaaacagggtgactaccagaaagccattgaagattatcaaaaggctttggaattggatcctaacaacagatctagatcagctggtggtggtggttctggtggtggtggttctggtggtggtggtgcttcttcatattaccatcaccatcaccatcacttggaatccacaagtttatacaaaaaggctggttctggttcaaatttggtcgcacaattggaaaacgaagtagcctctttagaaaatgaaaacgaaaccttgaaaaagaaaaacttacataagaaagatttgatcgcttatttggaaaaggaaatcgcaaatttgagaaagaaaattgaagaaggtagtgcaggttctgccgctggttctggtgaatttggttcagctgaagcagccgctaaggaagcagccgctaaagccggttcagctggttccgcagccggttctggtgaattcggttccagttactatcaccatcaccatcatcacttggaatccacaagtttatataagaaagcaggttctggttcagcaagaaatgcctacttgagaaagaaaatagctagattaaagaaagataacttgcaattggaaagagatgaacaaaatttggaaaagattatcgccaacttaagagatgaaatcgctagattggaaaatgaagttgcatcccatgaacaaggtagtggtgctactaacttctctttgttgaagcaagcaggtgacgttgaagaaaatccaggtccaatgaaaaactgtgtaatcgtttctgctgttagaactgcaattggttcctttaatggtagtttggcctctacatcagctattgatttgggtgctaccgtcatcaaagctgcaattgaaagagcaaagattgattctcaacatgtcgacgaagtaataatgggtaacgttttgcaagctggtttaggtcaaaatccagcaagacaagccttgttaaaatctggtttagcagaaactgtatgtggtttcacagttaataaggtctgcggttctggtttgaagtcagttgctttagccgctcaagctatacaagcaggtcaagcccaatctatcgtcgctggtggtatggaaaatatgtcattggcaccttatttgttagatgcaaaagccagatcaggttatagattaggtgacggtcaagtatacgacgttattttgagagatggtttaatgtgcgctactcatggttatcacatgggtattacagcagaaaatgttgccaaagaatacggtataaccagagaaatgcaagatgaattggcattacattcccaaagaaaggcagccgctgcaatcgaaagtggtgcttttactgcagaaattgtcccagtaaacgttgtcacaagaaagaaaactttcgttttctcccaagatgaattcccaaaagctaatagtaccgctgaagcattgggtgctttaagacctgcattcgacaaggccggtaccgtaactgccggtaatgcttctggtataaacgatggtgccgctgcattggttatcatggaagaatcagccgctttagcagccggtttgacacctttagctagaattaaatcttatgcatcaggtggtgttccacctgctttgatgggtatgggtccagtccctgctacccaaaaggcattgcaattagccggtttgcaattggctgatatcgacttaatcgaagcaaacgaagcctttgctgcacaattcttggcagttggtaaaaatttgggtttcgactccgaaaaggttaatgtcaacggtggtgccattgctttgggtcatccaataggtgcttcaggtgcaagaatcttggttacattgttgcatgccatgcaagctagagataaaaccttgggtttagctactttgtgtatcggtggtggtcaaggtatcgcaatggttatcgaaagattgaataagttgtctggtggtggtggttctggtggtggtggttctggtggtggtggtagtgcagaagcctggtacaatttgggtaacgcttactacaagcagggtgactaccaaaaggcaatcgaatactaccaaaaggccttggaattagatccaaataacgctgaagcatggtataatttgggtaatgcctattataaacagggtgactatcaaaaagctattgaatattaccaaaaggcattggaattagatcctaataacgccgaagcttggtataatttgggtaacgcctattataagcagggtgactatcaaaaggccatcgaagattaccaaaaggctttggaattggatccaaacaacttgcaagcagaagcctggaagaatttgggtaacgcttattacaaacagggtgactaccaaaaagctattgaatactatcaaaaagccttagaattggatcctaataacgcttctgcatggtacaatttgggtaatgcctactataaacagggtgactaccagaaggctattgaatactaccaaaaagcattagaattggatccaaataacgccaaggcttggtacagacgtggtaatgcctattacaagcagggtgactaccagaaagccatagaagactatcaaaaagccttggaattggatcctaacaacagatccagaagtgctggtggtggtggttctggtggtggtggttctggtggtggtggtgcttcttcatattaccatcaccatcaccatcacttggaatctacatcattatacaaaaaggctggttccggtagtaatgaagttactacattggaaaacgatgccgcttttatcgaaaacgaaaacgcatacttggaaaaggaaatcgccagattaagaaaggaaaaggcagccttgagaaatagattagcccataaaaagggttccgctggtagtgctgcaggttctggtgaatttggttcagctgaagccgctgcaaaagaagccgctgcaaaggcaggttctgccggttcagccgctggttctggtgaattcggttccagttactatcaccatcaccatcatcacttggaatctacttcattatataaaaaggccggttccggtagtcaaaaagtcgctgaattaaagaacagagtagctgttaagttgaacagaaacgaacaattgaaaaataaggtagaagaattgaaaaatagaaacgcctacttaaagaatgaattggcaacattggaaaacgaagtcgctagattggaaaatgatgtagcagaaggttctggt

SEQ ID NO: 207 - DNA, 7125 bp, synthetic construct
  source 1..7125: mol_type = other DNA
atgagtgctaaggcaatttctgaacaaactggtaaagaattgttgtacaagtttatttgtactacatcagccatccaaaatagattcaaatacgctagagttaccccagatactgactgggctagattgttacaagatcatccatggttgttatctcaaaacttggttgtcaaacctgaccaattaattaagagaagaggtaaattgggtttagtaggtgttaatttgacattggatggtgtaaagtcttggttgaaaccaagattaggtcaagaagccacagttggtaaagctaccggtttcttgaaaaatttcttgatcgaaccatttgtccctcattcacaagccgaagaattctatgtatgtatctacgctactagagagggtgactatgttttatttcatcacgaaggtggtgtcgacgtaggtgacgttgacgccaaggctcaaaagttgttggttggtgtcgatgaaaagttgaacccagaagacattaaaaagcatttgttggttcacgcacctgaagataaaaaggaaatattggcctcctttataagtggtttgtttaatttctacgaagatttgtacttcacctacttggaaattaacccattagtagttactaaggatggtgtatatgttttggacttagctgcaaaagttgatgcaacagccgactacatttgtaaggtcaaatggggtgacatcgaatttccacctccattcggtagagaagcttatccagaagaagcctacattgctgatttggacgctaagtctggtgcatcattgaagttgacattgttgaaccctaaaggtagaatttggaccatggttgctggtggtggtgctagtgtcgtatattctgatactatatgcgacttgggtggtgttaacgaattggcaaactacggtgaatactcaggtgccccatccgaacaacaaacatacgattacgctaagaccatcttgtccttaatgactagagaaaagcatcctgatggtaaaatcttgatcatcggtggtagtatcgcaaattttactaacgttgccgctacattcaagggtatcgtcagagctataagagattaccaaggtccattgaaggaacacgaagtaacaatattcgttagaagaggtggtcctaactaccaagaaggtttgagagtcatgggtgaagtaggtaaaaccactggtataccaatccatgtctttggtacagaaacccacatgactgcaatagttggtatggccttaggtcatagaccaatccctaatcaacctccaaccgcagcccacactgcaaatttcttgttaaacgcctctggttcaacttccacaccagctccttctagaacagcaagtttctctgaatcaagagctgatgaagtcgctccagctaagaaagcaaaaccagccatgcctcaagactccgttccaagtcctagatctttgcagggtaaatctactactttgttttctagacatactaaggctatagtatggggtatgcaaacaagagcagttcaaggcatgttggatttcgactatgtttgtagtagagatgaaccatctgttgctgcaatggtctatccttttactggtgaccataagcaaaaattctactggggtcacaaggaaatattgatcccagtttttaagaacatggccgatgctatgagaaaacatcctgaagtcgacgtattgattaacttcgcctcattaagatccgcttacgattctacaatggaaaccatgaactacgctcaaataagaaccatcgctatcattgcagaaggtattccagaagccttgactagaaagttgattaagaaagctgatcaaaaaggtgtcacaataatcggtccagctaccgtaggtggtattaagcctggttgtttcaagatcggtaacactggtggtatgttggataacatattggcatctaagttgtatagaccaggttcagtcgcttacgtatccagaagtggtggtatgtccaacgaattgaacaacatcatcagtagaactacagatggtgtatacgaaggtgttgctattggtggtgacagatacccaggttctacttttatggatcatgtattgagatatcaagacacacctggtgttaaaatgattgttgtcttgggtgaaataggtggtactgaagaatacaagatatgcagaggtatcaaagaaggtagattgacaaagccaatcgtttgttggtgcattggtacttgtgcaacaatgttttcttcagaagttcaattcggtcatgcaggtgcctgcgctaatcaagcttcagaaacagcagttgccaagaaccaagcattaaaagaagccggtgtttttgtccctagatctttcgatgaattaggtgaaatcattcaatcagtctatgaagacttggtagctaatggtgtaattgttccagcacaagaagttcctccacctactgtccctatggattactcttgggcaagagaattgggtttaattagaaagccagctagttttatgacctctatatgtgatgaaagaggtcaagaattgatctatgctggtatgcctattactgaagtattcaaagaagaaatgggtatcggtggtgttttaggtttgttgtggttccaaaagagattgccaaagtactcttgtcaattcattgaaatgtgcttaatggttacagctgatcatggtcctgctgtctcaggtgcacacaataccataatctgcgctagagctggtaaagatttggtttcttctttgacctcaggtttgttaactattggtgacagatttggtggtgcattagacgccgctgcaaagatgttttcaaaagctttcgattccggtataatcccaatggaattcgttaataagatgaaaaaggagggtaaattgataatgggtatcggtcatcgtgttaagtctatcaataaccctgatatgagagtacaaatcttgaaggactatgttagacaacactttccagccacacctttgttagattacgctttggaagttgaaaagattaccacttctaaaaagccaaatttgatcttgaacgttgatggtttaattggtgttgcttttgtcgacatgttgagaaactgtggttccttcactagagaagaagctgatgaatatatcgacattggtgcattgaatggtatctttgttttaggtagatctatgggtttcattggtcattacttggatcaaaagagattaaagcaaggtttgtacagacatccatgggatgacatttcttacgttttacctgaacacatgtcaatgaaattgtctggtggtggtggttctggtggtggtggttctggtggtggtggtagtgccgaagcttggtacaatttgggtaacgcatactacaagcagggtgactaccaaaaggcaattgaatattaccaaaaggccttggaattagacccaaataacgcagaagcctggtataatttgggtaatgcttattataaacagggtgactatcaaaaggctatcgaatactaccaaaaggcattggaattagaccctaataacgctgaagcatggtataatttgggtaacgcttattataagcagggtgactatcaaaaagccatcgaagactaccaaaaggctttggaattagatccaaataacttacaagccgaagcttggaagaatttgggtaacgcttactataaacagggtgactaccaaaaagcaattgaatactatcaaaaagctttagaattggaccctaataacgcatcagcctggtacaatttgggtaatgcttactataagcagggtgactatcagaaggccattgaatactatcaaaaggctttagaattggatccaaataacgctaaagcatggtacagacgtggtaacgcttattacaaacagggtgactaccagaaagccattgaagattatcaaaaggctttggaattggatcctaacaacagatctagatcagctggtggtggtggttctggtggtggtggttctggtggtggtggtgcttcttcatattaccatcaccatcaccatcacttggaatccacaagtttatacaaaaaggctggttctggttcaaatttggtcgcacaattggaaaacgaagtagcctctttagaaaatgaaaacgaaaccttgaaaaagaaaaacttacataagaaagatttgatcgcttatttggaaaaggaaatcgcaaatttgagaaagaaaattgaagaaggtagtgcaggttctgccgctggttctggtgaatttggttcagctgaagcagccgctaaggaagcagccgctaaagccggttcagctggttccgcagccggttctggtgaattcggttccagttactatcaccatcaccatcatcacttggaatccacaagtttatataagaaagcaggttctggttcagcaagaaatgcctacttgagaaagaaaatagctagattaaagaaagataacttgcaattggaaagagatgaacaaaatttggaaaagattatcgccaacttaagagatgaaatcgctagattggaaaatgaagttgcatcccatgaacaaggtagtggtgctactaacttctctttgttgaagcaagcaggtgacgttgaagaaaatccaggtccaatgaaaaactgtgtaatcgtttctgctgttagaactgcaattggttcctttaatggtagtttggcctctacatcagctattgatttgggtgctaccgtcatcaaagctgcaattgaaagagcaaagattgattctcaacatgtcgacgaagtaataatgggtaacgttttgcaagctggtttaggtcaaaatccagcaagacaagccttgttaaaatctggtttagcagaaactgtatgtggtttcacagttaataaggtctgcggttctggtttgaagtcagttgctttagccgctcaagctatacaagcaggtcaagcccaatctatcgtcgctggtggtatggaaaatatgtcattggcaccttatttgttagatgcaaaagccagatcaggttatagattaggtgacggtcaagtatacgacgttattttgagagatggtttaatgtgcgctactcatggttatcacatgggtattacagcagaaaatgttgccaaagaatacggtataaccagagaaatgcaagatgaattggcattacattcccaaagaaaggcagccgctgcaatcgaaagtggtgcttttactgcagaaattgtcccagtaaacgttgtcacaagaaagaaaactttcgttttctcccaagatgaattcccaaaagctaatagtaccgctgaagcattgggtgctttaagacctgcattcgacaaggccggtaccgtaactgccggtaatgcttctggtataaacgatggtgccgctgcattggttatcatggaagaatcagccgctttagcagccggtttgacacctttagctagaattaaatcttatgcatcaggtggtgttccacctgctttgatgggtatgggtccagtccctgctacccaaaaggcattgcaattagccggtttgcaattggctgatatcgacttaatcgaagcaaacgaagcctttgctgcacaattcttggcagttggtaaaaatttgggtttcgactccgaaaaggttaatgtcaacggtggtgccattgctttgggtcatccaataggtgcttcaggtgcaagaatcttggttacattgttgcatgccatgcaagctagagataaaaccttgggtttagctactttgtgtatcggtggtggtcaaggtatcgcaatggttatcgaaagattgaataagttgtctggtggtggtggttctggtggtggtggttctggtggtggtggtagtgcagaagcctggtacaatttgggtaacgcttactacaagcagggtgactaccaaaaggcaatcgaatactaccaaaaggccttggaattagatccaaataacgctgaagcatggtataatttgggtaatgcctattataaacagggtgactatcaaaaagctattgaatattaccaaaaggcattggaattagatcctaataacgccgaagcttggtataatttgggtaacgcctattataagcagggtgactatcaaaaggccatcgaagattaccaaaaggctttggaattggatccaaacaacttgcaagcagaagcctggaagaatttgggtaacgcttattacaaacagggtgactaccaaaaagctattgaatactatcaaaaagccttagaattggatcctaataacgcttctgcatggtacaatttgggtaatgcctactataaacagggtgactaccagaaggctattgaatactaccaaaaagcattagaattggatccaaataacgccaaggcttggtacagacgtggtaatgcctattacaagcagggtgactaccagaaagccatagaagactatcaaaaagccttggaattggatcctaacaacagatccagaagtgctggtggtggtggttctggtggtggtggttctggtggtggtggtgcttcttcatattaccatcaccatcaccatcacttggaatctacatcattatacaaaaaggctggttccggtagtaatgaagttactacattggaaaacgatgccgcttttatcgaaaacgaaaacgcatacttggaaaaggaaatcgccagattaagaaaggaaaaggcagccttgagaaatagattagcccataaaaagggttccgctggtagtgctgcaggttctggtgaatttggttcagctgaagccgctgcaaaagaagccgctgcaaaggcaggttctgccggttcagccgctggttctggtgaattcggttccagttactatcaccatcaccatcatcacttggaatctacttcattatataaaaaggccggttccggtagtcaaaaagtcgctgaattaaagaacagagtagctgttaagttgaacagaaacgaacaattgaaaaataaggtagaagaattgaaaaatagaaacgcctacttaaagaatgaattggcaacattggaaaacgaagtcgctagattggaaaatgatgtagcagaaggttctggt

SEQ ID NO: 208 - protein, 7 aa, synthetic construct
GVKESLV

SEQ ID NO: 209 - protein, 723 aa, synthetic construct
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFEGSG

SEQ ID NO: 210 - protein, 127 aa, synthetic construct
GSHMGSQFWVTSQKTEASERCGLQGSYILRVEAEKLTLLTLGAQSQILEPLLFWPYTLLRRYGRDKVMFSFEAGRRCPSGPGTFTFQTSQGNDIFQAVEAAIQQQKAQGKVGQAQDILRLEHHHHHH

SEQ ID NO: 211 - protein, 2741 aa, synthetic construct
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLSDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVSVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLKKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGASGSHMRLGAQSIQPTANLDRTDDLVYLNVMELVRAVLELKNELAQLPPEGYVVVVKNVGLTLRKLIGSVDDLLPSLPSSSRTEIEGTQKLLNKDLAELINKMRLAQQNAVTSLSEECKRQMLTASHTLAVDAKNLLDAVDQAKVLANLAHPPAEGSAGSAAGSGEFGSAEAAAKEAAAKAGSAGSAAGSGEFGSGAMATPGSENVLPREPLIATAVKFLQNSRVRQSPLATRRAFLKKKGLTDEEIDMAFQQSGTAADEPSSLW

SEQ ID NO: 212 - DNA, 57 bp, synthetic construct
  source 1..57: mol_type = other DNA
gctactaacttctctttgttgaagcaagcaggtgacgttgaagaaaatccaggtcca

(19) United States
(12) Patent Application Publication
(10) Pub. No.: US 2025/0361532 A1
(43) Pub. Date: Nov. 27, 2025

(54) BIDIRECTIONAL MULTI-ENZYMATIC SCAFFOLDS FOR BIOSYNTHESIZING CANNABINOIDS

(71) Applicant: Khona Scientific Holdings, Inc., Lone Tree, CO (US)

(72) Inventor: Jordan Buck, Boulder, CO (US)

(21) Appl. No.: 19/264,249

(22) Filed: Jul. 9, 2025

Related U.S. Application Data
(63) Continuation of application No. 17/962,229, filed on Oct. 7, 2022, now Pat. No. 12,385,072.
Application No. 17/962,229 is a continuation of application No. 16/694,417, filed on Nov. 25, 2019, now Pat. No. 11,525,148.
(60) Provisional application No. 62/771,839, filed on Nov. 27, 2018.
(60) Provisional application No. 62/836,265, filed on Apr. 19, 2019.

(51) Int. Cl. C12P 7/42; C12N 15/70; C12N 15/74; C12N 15/81; C12N 15/82

(52) U.S. Cl. CPC C12P 7/42; C12N 15/70; C12N 15/74; C12N 15/81; C12N 15/8222; C12N 2330/51; C12N 2800/40; C12Y 101/01034; C12Y 101/01157; C12Y 103/01038; C12Y 103/03; C12Y 121/03007; C12Y 121/03008; C12Y 203/01009; C12Y 203/01016; C12Y 203/01206; C12Y 203/03008; C12Y 203/0301; C12Y 205/01001; C12Y 207/01036; C12Y 207/04002; C12Y 401/01033; C12Y 402/01017; C12Y 404/01026; C12Y 503/03002; C12Y 604/01002

(57) ABSTRACT

This document relates to using bidirectional, multi-enzymatic scaffolds to biosynthesize cannabinoids in recombinant hosts.

### CROSS-REFERENCE TO RELATED APPLICATIONS

This application is a continuation of U.S. application Ser. No. 17/962,229, filed on Oct. 7, 2022, which is a continuation of U.S. application Ser. No. 16/694,417, filed on Nov. 25, 2019, now U.S. Pat. No. 11,525,148, which claims priority to U.S. Application Ser. No. 62/836,265, filed on Apr. 19, 2019, and U.S. Application Ser. No. 62/771,839, filed on Nov. 27, 2018. The disclosures of the prior applications are considered part of (and are incorporated by reference in) the disclosure of this application.

### SEQUENCE LISTING

This application contains a Sequence Listing that has been submitted electronically as an XML file named “47300-0003002 SL ST26.XML.” The XML file, created on May 12, 2023, is 487,591 bytes in size. The material in the XML file is hereby incorporated by reference in its entirety.

### TECHNICAL FIELD

This document relates to methods and materials for biosynthesizing cannabinoids, and more particularly to using bidirectional multi-enzymatic scaffolds to biosynthesize cannabinoids.

### BACKGROUND

The emerging therapeutic potential of cannabinoids warrants industrial-scale production to meet compounding future demands. Traditional cannabinoid production efforts rely on large-scale farming of Cannabis sativa L. However, agricultural cannabinoid production is problematic due to issues such as uncontrollable environmental factors and scaling limitations.

### SUMMARY

This document is based, at least in part, on the discovery that a bidirectional, multi-enzymatic scaffold can be engineered to allow high-throughput cannabinoid production in recombinant host cells. By controlling the localization, spatial orientation, and stoichiometry of enzymes catalyzing the biosynthesis of cannabinoids and cannabinoid precursors, the multi-enzymatic scaffolds described herein allow flux-optimized cannabinoid biosynthesis in genetically-engineered host cells.

In one aspect, this document features a host cell capable of producing one or more cannabinoids selected from the group consisting of cannabigerolic acid, cannabidiolic acid, and cannabichromenic acid. The host cell includes at least three different exogenous nucleic acids, wherein the first and the second exogenous nucleic acids each encode a plurality of engineered enzymes selected from the group consisting of acetyl-CoA acetyltransferase, a 3-hydroxybutyryl-CoA dehydrogenase, an enoyl-CoA hydratase, a beto-ketothiolase, a trans-enoyl-CoA reductase, an HMG-CoA synthetase, an HMG-CoA reductase, a mevalonate kinase, a phosphomevalonate kinase, a diphosphomevalonate decarboxylase, an isopentenyl-diphosphate delta isomerase, a geranyl-diphosphate synthase, an olivetol synthase, an olivetolic acid cyclase, and a CBGA synthase; wherein each of the engineered enzymes includes a heterologous interaction domain, wherein the heterologous interaction domain comprises a first and a second peptide motif, and wherein each heterologous interaction domain is different from each other; and wherein the third exogenous nucleic acid encodes a polypeptide scaffold comprising a plurality of peptide ligands, wherein each peptide ligand comprises an amino acid sequence that can bind to the first or the second peptide motif of one of the heterologous interaction domains. The plurality of engineered enzymes further can include an ATP citrate lyase and an acetyl-CoA carboxylase. The host cell further can include an exogenous nucleic acid encoding a cannabidiolic acid synthase (CBDAS) and a cannabichromenic acid synthase (CBCAS). The host cell can include an exogenous CBDAS. The host cell can include an exogneous CBCAS. The host cell can include an exogenous CBDAS and an exogenous CBCAS. The host cell can include an exogenous hexanoyl-CoA synthetase. The host cell can include at least four different exogenous nucleic acids, wherein the first, second, and fourth nucleic acids each encode a plurality of the engineered enzymes. The host cell can include at least five different exogenous nucleic acids, wherein the first, second, fourth, and fifth nucleic acid each encode a plurality of the engineered enzymes. The host cell can include at least six different exogenous nucleic acids, wherein the first, second, fourth, fifth, and sixth nucleic acids each encode a plurality of the engineered enzymes. Each exogenous nucleic acid can include a constitutive promoter operably linked to the sequence encoding the engineered enzyme or polypeptide scaffold or an inducible promoter operably linked to the sequence encoding the engineered enzyme or polypeptide scaffold. In some embodiments, the promoter is a GAL1-10 promoter. In some embodiments, a constitutive promoter used to express the polypeptide scaffold has weaker constitutive activity level than a constitutive promoter used to express the engineered enzymes. In some embodiments, a constitutive promoter is used to express the engineered enzymes and an inducible promoter is used to express the polypeptide scaffold. In some embodiments, an inducible promoter is used to express the engineered enzymes and a constitutive promoter is used to express the polypeptide scaffold.

Any of the host cells can be bacterial, yeast, algae, or plant cells. A bacterial cell can be selected from the group consisting of Escherichia coli, Bacillus, Brevibacterium, Streptomyces, and Pseudomonas cells. A yeast cell can be selected from the group consisting of Pichia pastoris, Saccharomyces cerevisiae, Yarrowia lipolytica, Kluyveromyces marxianus, and Komagataella phaffii cells. An algae cell can be Dunaliella sp., Chlorella variabilis, Euglena mutabilis, or Chlamydomonas reinhardtii cells. A plant cell can be a Cannabis or tobacco cell.

In some embodiments, each of the engineered enzymes is of the formula: enzyme—linker₁—spacer—linker₂—motif₁—linker₃—motif₂, where linkers 1, 2, and 3 can be the same or different, motif 1 and motif 2 can be the same or different, and where motif 1 and motif 2 form the heterologous interaction domain. A scaffold polypeptide can be of the formula: N-terminus—[Ligand 1—linker—Ligand 2—Spacer]n—(optionally-tagged) C-terminus, where n is the number of heterologous interaction domains, and where ligand 1 and ligand 2 bind motif 1 and motif 2, respectively, of the heterologous interaction domain. The scaffold polypeptide can be tagged with a MYC tag, FLAG tag, or HA tag. The host cell further can include a nucleic acid encoding a second polypeptide scaffold comprising a plurality of peptide ligands, wherein each peptide ligand comprises an amino acid sequence that can bind to a different motif of the heterologous interaction domain. The linker can have a flexible GS-rich sequence flanking a rigid α-helical moiety. The spacer can be the cTPR6 spacer.

This document also features a method of producing one or more cannabinoids selected from the group consisting of cannabigerolic acid, cannabidiolic acid, and cannabichromenic acid. The method can include culturing any of the host cells described herein under conditions wherein the host cell produces the one or more cannabinoids. The host cells can be cultured in a culture medium supplemented with citrate, glucose, hexanoic acid, and/or other carbon source, and/or in a culture medium supplemented with malonyl-CoA. The method further can include extracting the one or more cannabinoids from the host cells.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a schematic of one representative embodiment of a multi-enzymatic cannabinoidergic scaffold within a cell. The multi-enzymatic scaffold includes enzymes of the hexanoyl-CoA pathway, enzymes of the upper cannabinoid pathway, and enzymes of the mevalonate pathway. The schematic also depicts a second scaffold according to one embodiment containing enzymes of the malonyl-CoA pathway and depicts a non-scaffolded cannabidiolic acid synthase (CBDAS) and a non-scaffolded cannabichromenic acid synthase (CBCAS). ID refers to enzyme-linked interaction domain; cTPR6 refers to a spacer sequence; scaffolded ligands refer to the tandem peptide ligands that form the scaffold-binding sites specific for each enzyme-linked ID. The target products cannabigerolic acid (CBGA), cannabigerol (CBG), cannabidiolic acid (CBDA), cannabidiol (CBD), cannabichromenic acid (CBCA), and cannabichromene (CBC), are boxed for emphasis. CBG can be produced by decarboxylation of CBGA, CBD can be produced by decarboxylation of CBDA, and CBC can be produced by decarboxylation of CBCA. For each decarboxylation, the ‘Δ’ symbols represent heat and the ‘hv’ symbols represent light.

FIG. 1B is a schematic of one representative embodiment of a bidirectional, multi-enzymatic scaffold within a cell (e.g., a yeast cell). The multi-enzymatic scaffold (referred to as SCF gene cassette in the nucleus) includes enzymes of the hexanoyl-CoA pathway (referred to as HCA cassette in nucleus), enzymes of the upper cannabinoid pathway (referred to as CAN cassette in nucleus), and enzymes of the mevalonate pathway (referred to as GPP cassette in nucleus). The schematic also depicts a second scaffold according to one embodiment containing enzymes of the malonyl-CoA pathway and depicts a non-scaffolded CBDAS and a non-scaffolded CBCAS. ID refers to enzyme-linked interaction domain; cTPR6 refers to a spacer sequence; scaffolded ligands refer to the tandem peptide ligands that form the scaffold-binding sites specific for each enzyme-linked ID. The target products CBGA, CBG, CBDA, CBD, CBCA, and CBC are boxed for emphasis. CBG can be produced by decarboxylation of CBGA, CBD can be produced by decarboxylation of CBDA, and CBC can be produced by decarboxylation of CBCA. For each decarboxylation, the ‘Δ’ symbols represent heat and the ‘hv’ symbols represent light.

FIG. 2A is a schematic of gene cassettes according to one embodiment for the engineering of cannabinoidergic cells.

FIG. 2B is a schematic of gene cassettes used in Examples 2-4 for biosynthesizing cannabinoids in yeast.

FIG. 3 is an example of an enzyme-scaffold complex.

FIG. 4 is a schematic of one representative embodiment of a multi-enzymatic cannabinoidergic scaffold within a cell. The multi-enzymatic scaffold includes enzymes of the hexanoyl-CoA pathway, enzymes of the upper cannabinoid pathway, and enzymes of the mevalonate pathway. The schematic also depicts a second scaffold according to one embodiment containing enzymes of the malonyl-CoA pathway and depicts a non-scaffolded CBDAS and a non-scaffolded CBCAS. Pyruvate dehydrogenase (E1) and dihydrolipoyl transacetylase (E2) are substituted for ATP citrate lyase in both of the depicted scaffolds. ID refers to enzyme-linked interaction domain; cTPR6 refers to a spacer sequence; scaffolded ligands refer to the tandem peptide ligands that form the scaffold-binding sites specific for each enzyme-linked ID. The target products CBGA, CBG, CBDA, CBD, CBCA, and CBC are boxed for emphasis. CBG can be produced by decarboxylation of CBGA, CBD can be produced by decarboxylation of CBDA, and CBC can be produced by decarboxylation of CBCA. For each decarboxylation, the ‘Δ’ symbols represent heat and the ‘hv’ symbols represent light.

FIG. 5 is a schematic of one representative embodiment of a multi-enzymatic cannabinoidergic scaffold within a cell. The multi-enzymatic scaffold includes enzymes of the hexanoyl-CoA pathway, enzymes of the upper cannabinoid pathway, and enzymes of the MEP (2-C-methylerythritol 4-phosphate) pathway. The schematic also depicts a second scaffold according to one embodiment containing enzymes of the malonyl-CoA pathway and depicts a non-scaffolded CBDAS and a non-scaffolded CBCAS. ID refers to enzyme-linked interaction domain; cTPR6 refers to a spacer sequence; scaffolded ligands refer to the tandem peptide ligands that form the scaffold-binding sites specific for each enzyme-linked ID. The target products CBGA, CBG, CBDA, CBD, CBCA, and CBC are boxed for emphasis. CBG can be produced by decarboxylation of CBGA, CBD can be produced by decarboxylation of CBDA, and CBC can be produced by decarboxylation of CBCA. For each decarboxylation, the ‘Δ’ symbols represent heat and the ‘hv’ symbols represent light.

FIG. 6A contains the amino acid sequence of each of the following enzymes: an ATP citrate lyase (SEQ ID NO:83), acetyl-CoA acetyltransferase (atoB) (SEQ ID NO:84), a 3-hydroxybutyryl-CoA dehydrogenase (SEQ ID NO:85), an enoyl-CoA hydratase (SEQ ID NO:86), a trans-enoyl-CoA reductase (SEQ ID NO:88), a beta-ketothiolase (bktB) (SEQ ID NO:87), an HMG-CoA synthase (SEQ ID NO:90), a truncated HMG-CoA reductase (SEQ ID NO:91), a mevalonate kinase (SEQ ID NO:92), a phosphomevalonate kinase (SEQ ID NO:93), a diphosphomevalonate decarboxylase (SEQ ID NO:94), an isopentenyl-diphosphate delta isomerase (SEQ ID NO:95), a mutant geranyl-diphosphate synthase (ERG20^{WW}) (SEQ ID NO:96), an olivetol synthase (SEQ ID NO:98), an olivetolic acid cyclase (SEQ ID NO:99), a CBGA synthase (SEQ ID NO:100), an acetyl-CoA carboxylase (SEQ ID NO:97), a CBDA synthase (SEQ ID NO:101), a CBCA synthase (SEQ ID NO:102), and a hexanoyl-CoA synthetase (SEQ ID NO:89).

FIG. 6B contains the amino acid sequence of engineered enzymes of the formula Enzyme—Enzyme Linker—cTPR6 Spacer—ID Linker—ID Motif #1—ID Motif Linker—ID Motif #2, where the linkers (enzyme linker, ID linker, and ID motif linker) can be the same or different, and ID motif #1 and ID motif #2 can be the same or different. The amino acid sequence of the following engineered enzymes are provided: ATP citrate lyase (ID1) (SEQ ID NO:103), an acetyl-CoA acetyltransferase (atoB) (ID2) (SEQ ID NO:104), a 3-hydroxybutyryl-CoA dehydrogenase (ID3) (SEQ ID NO:105), an enoyl-CoA hydratase (ID4) (SEQ ID NO:106), a trans-enoyl-CoA reductase (ID5) (SEQ ID NO:107), a beto-ketothiolase (bktB) (ID6) (SEQ ID NO:108), an HMG-CoA synthase (ID7) (SEQ ID NO:109), a truncated HMG-CoA reductase (ID8) (SEQ ID NO:110), a mevalonate kinase (ID9) (SEQ ID NO:111), a phosphomevalonate kinase (ID10) (SEQ ID NO:112), a diphosphomevalonate decarboxylase (ID11) (SEQ ID NO:113), an isopentenyl-diphosphate delta isomerase (ID12) (SEQ ID NO:114), a mutant geranyl-diphosphate synthase (ERG20^{WW}) (ID13) (SEQ ID NO:115), an olivetol synthase (ID14) (SEQ ID NO:116), an olivetolic acid cyclase (ID15) (SEQ ID NO:117), a CBGA synthase (ID16) (SEQ ID NO:118), and an acetyl-CoA carboxylase (ID17) (SEQ ID NO:211).

FIG. 6C contains the amino acid sequence of a polypeptide scaffold of the formula: N-terminus—[Ligand #1—ID Motif #1 Ligand—Linker—ID Motif #2 Ligand—Scaffolded ID—binding Site Spacer]n—(Myc)3-tagged C-terminus, where n is 16 and the ID motif ligands correspond to the motifs for IDs 1-16 as shown in Table 2. See SEQ ID NO:119.

FIG. 6D contains the amino acid sequence of a polypeptide scaffold of the formula: N-terminus—[Ligand #1—ID Motif #1 Ligand—Linker—ID Motif #2 Ligand—Scaffolded ID—binding Site Spacer]n—(FLAG)3-tagged C-terminus, where n is 2 and the ID motif ligands correspond to the motifs for IDs 1 and 17 as shown in Table 2. See SEQ ID NO:120.

FIG. 7 is a schematic of one representative embodiment of a scaffold with the minimal requirements for cannabigerolic acid synthesis. The scaffold contains enzymes of the upper cannabinoid pathway. In this embodiment, a non-scaffolded hexanoyl-CoA synthetase (HCS), a non-scaffolded CBDAS, and a non-scaffolded CBCAS also are used. ID refers to enzyme-linked interaction domain; cTPR6 refers to a spacer sequence; scaffolded ligands refer to the tandem peptide ligands that form the scaffold-binding sites specific for each enzyme-linked ID. The target products CBGA, CBG, CBDA, CBD, CBCA, and CBC are boxed for emphasis. CBG can be produced by decarboxylation of CBGA, CBD can be produced by decarboxylation of CBDA, and CBC can be produced by decarboxylation of CBCA. For each decarboxylation, the ‘Δ’ symbols represent heat and the ‘hv’ symbols represent light.

FIG. 8 is a schematic of one representative embodiment of a bi-directional scaffold containing a HCS on the N-terminus of the scaffold, a geranyl pyrophosphate synthase (GPPS) on the C-terminus of the scaffold, and the enzymes of the upper cannabinoid pathway between the HCS and GPPS. In this embodiment, a non-scaffolded CBDAS and a non-scaffolded CBCAS also can be used. ID refers to enzyme-linked interaction domain; cTPR6 refers to a spacer sequence; scaffolded ligands refer to the tandem peptide ligands that form the scaffold-binding sites specific for each enzyme-linked ID. The target products CBGA, CBG, CBDA, CBD, CBCA, and CBC are boxed for emphasis. CBG can be produced by decarboxylation of CBGA, CBD can be produced by decarboxylation of CBDA, and CBC can be produced by decarboxylation of CBCA. For each decarboxylation, the ‘Δ’ symbols represent heat and the ‘hv’ symbols represent light.

FIG. 9 is a schematic of one representative embodiment of a unidirectional scaffold containing enzymes of the upper cannabinoid pathway, shown with soluble enzymes from the precursor pathways (hexanoyl-CoA pathway, mevalonate pathway, and malonyl-CoA pathway), and soluble CBDAS and CBCAS. ID refers to enzyme-linked interaction domain; cTPR6 refers to a spacer sequence; scaffolded ligands refer to the tandem peptide ligands that form the scaffold-binding sites specific for each enzyme-linked ID. The target products CBGA, CBG, CBDA, CBD, CBCA, and CBC are boxed for emphasis. CBG can be produced by decarboxylation of CBGA, CBD can be produced by decarboxylation of CBDA, and CBC can be produced by decarboxylation of CBCA. For each decarboxylation, the ‘Δ’ symbols represent heat and the ‘hv’ symbols represent light.

FIG. 10 is a schematic of one representative embodiment of a multi-enzymatic cannabinoidergic scaffold within a cell. The multi-enzymatic scaffold includes enzymes of the malonyl-CoA (MCA) pathway, enzymes of the upper cannabinoid pathway, and enzymes of the mevalonate pathway. The schematic also depicts a separate scaffold according to one embodiment containing enzymes of the hexanoyl-CoA pathway and depicts a non-scaffolded CBDAS and a non-scaffolded CBCAS. ID refers to enzyme-linked interaction domain; cTPR6 refers to a spacer sequence; scaffolded ligands refer to the tandem peptide ligands that form the scaffold-binding sites specific for each enzyme-linked ID. The target products CBGA, CBG, CBDA, CBD, CBCA, and CBC are boxed for emphasis. CBG can be produced by decarboxylation of CBGA, CBD can be produced by decarboxylation of CBDA, and CBC can be produced by decarboxylation of CBCA. For each decarboxylation, the ‘Δ’ symbols represent heat and the ‘hv’ symbols represent light.

FIG. 11 is a schematic of one representative embodiment of a multi-enzymatic cannabinoidergic scaffold within dual compartments of a cell, the cytosol and mitochondria/plastid.

FIG. 12A contains the nucleotide sequences encoding each of the following: an ATP citrate lyase (SEQ ID NO:121), an acetyl-CoA acetyltransferase (atoB) (SEQ ID NO:122), a 3-hydroxybutyryl-CoA dehydrogenase (SEQ ID NO:123), an enoyl-CoA hydratase (SEQ ID NO:124), a trans-enoyl-CoA reductase (SEQ ID NO:125), a beto-ketothiolase (bktB) (SEQ ID NO:126), an HMG-CoA synthase (SEQ ID NO:127), a truncated HMG-CoA reductase (SEQ ID NO:128), a mevalonate kinase (SEQ ID NO:129), a phosphomevalonate kinase (SEQ ID NO:130), a diphosphomevalonate decarboxylase (SEQ ID NO:131), an isopentenyl-diphosphate delta isomerase (SEQ ID NO:132), a geranyl-diphosphate synthase (ERG20^{WW}) (SEQ ID NO:133), an olivetol synthase (SEQ ID NO:134), an olivetolic acid cyclase (SEQ ID NO:135), a CBGA synthase (SEQ ID NO:136), an acetyl-CoA carboxylase (SEQ ID NO:137), a CBDA synthase (SEQ ID NO:138), a CBCA synthase (SEQ ID NO:139), and a hexanoyl-CoA synthetase (SEQ ID NO:140).

FIG. 12B contains the nucleotide sequences encoding engineered enzymes of the formula: Enzyme—Enzyme Linker—cTPR6 Spacer—ID Linker—ID Motif #1—ID Motif Linker—ID Motif #2, where the Enzyme Linker, ID Linker, and ID Motif Linker can be the same or different, and where ID Motif #1 and ID Motif #2 can be the same or different. The nucleotide sequences encoding the following engineered enzymes are provided: ATP citrate lyase (ID1) (SEQ ID NO:141), an acetyl-CoA acetyltransferase (atoB) (ID2) (SEQ ID NO:142), a 3-hydroxybutyryl-CoA dehydrogenase (ID3) (SEQ ID NO:143), an enoyl-CoA hydratase (ID4) (SEQ ID NO:144), a trans-enoyl-CoA reductase (ID5) (SEQ ID NO:145), a bktB (ID6) (SEQ ID NO:146), an HMG-CoA synthase (ID7) (SEQ ID NO:147), a truncated HMG-CoA reductase (ID8) (SEQ ID NO:148), a mevalonate kinase (ID9) (SEQ ID NO:149), a phosphomevalonate kinase (ID10) (SEQ ID NO:150), a diphosphomevalonate decarboxylase (ID11) (SEQ ID NO:151), an isopentenyl-diphosphate delta isomerase (ID12) (SEQ ID NO:152), a mutant geranyl-diphosphate synthase (ERG20^{WW}) (ID13) (SEQ ID NO:153), an olivetol synthase (ID14) (SEQ ID NO:154), an olivetolic acid cyclase (ID15) (SEQ ID NO:155), a CBGA synthase (ID16) (SEQ ID NO:156), and an acetyl-CoA carboxylase (ID17) (SEQ ID NO:157).

FIG. 12C contains the nucleotide sequence (SEQ ID NO:158) encoding a scaffold polypeptide that contains the peptide ligands corresponding to IDs 1-16 as shown in Table 2 and a triplicate myc tag on the C-terminus.

FIG. 12D contains the nucleic acid sequence (SEQ ID NO:159) encoding a scaffold polypeptide that contains the peptide ligands corresponding to IDs 1 and 17, and a triplicate FLAG tag on the C-terminus.

FIG. 13A contains the amino acid sequence of scaffold-binding engineered enzymes and a soluble hexanoyl-CoA synthetase (HCS) (SEQ ID NO:209) encoded by the HCA gene cassette. The scaffold-binding engineered enzymes are ATP Citrate Lyase (ACL) (ACL—Enzyme—cTPR6 Spacer—ID Linker—ID1) (SEQ ID NO:160); Acetyl-CoA Acetyltransferase (atoB) (atoB—Enzyme Linker—cTPR6 Spacer—ID Linker—ID2) (SEQ ID NO:161); 3-Hydroxybutyryl-CoA Dehydrogenase (BHBD) (BHBD—Enzyme Linker—cTPR6 Spacer—ID Linker—ID3) (SEQ ID NO:162); Enoyl-CoA Hydratase (ECH) (ECH—Enzyme Linker—cTPR6 Spacer—ID Linker—ID4) (SEQ ID NO:163); Trans-Enoyl-CoA Reductase (ECR) (ECR—Enzyme Linker—cTPR6 Spacer—ID Linker—ID5) (SEQ ID NO:164); and Beta-Ketothiolase (bktB) (bktB—Enzyme Linker—cTPR6 Spacer—ID Linker—ID6) (SEQ ID NO:165).

FIG. 13B contains the amino acid sequences of scaffold-binding engineered enzymes encoded by the GPP gene cassette. The scaffold-binding engineered enzymes are HMG-CoA Synthase (HMGS) (HMGS—Enzyme Linker—cTPR6 Spacer—ID Linker—ID7) (SEQ ID NO:166); truncated HMG-CoA Reductase (tHMGR) (tHMGR—Enzyme Linker—cTPR6 Spacer—ID Linker—ID8) (SEQ ID NO:167); Mevalonate Kinase (ERG12) (ERG12—Enzyme Linker—cTPR6 Spacer—ID Linker—ID9) (SEQ ID NO:168); Phosphomevalonate Kinase (ERG8) (ERG8—Enzyme Linker—cTPR6 Spacer—ID Linker—ID10) (SEQ ID NO:169); Diphosphomevalonate Decarboxylase (MVD1) (MVD1—Enzyme Linker—cTPR6 Spacer—ID Linker—ID11) (SEQ ID NO:170); Isopentenyl-Diphosphate Delta-Isomerase (IDI1) (IDI1—Enzyme Linker—cTPR6 Spacer—ID Linker—ID12) (SEQ ID NO:171); and Geranyl-Diphosphate Synthase (ERG20WW) (ERG20WW—Enzyme Linker—cTPR6 Spacer—ID Linker—ID13) (SEQ ID NO:172).

FIG. 13C contains the amino acid sequences of scaffold-binding engineered enzymes, a soluble CBDA synthase (SEQ ID NO:173), and a soluble CBCA synthase (SEQ ID NO:174) encoded by the CAN gene cassette. The scaffold-binding engineered enzymes are Olivetol Synthase (OS) (OS—Enzyme Linker—cTPR6 Spacer—ID Linker—ID14) SEQ ID NO:175); Olivetolic Acid Cyclase (OAC) (OAC—Enzyme Linker—cTPR6 Spacer—ID Linker—ID15) (SEQ ID NO:176); CBGA Synthase (CBGAS—Enzyme Linker—cTPR6 Spacer—ID Linker—ID16) (SEQ ID NO:177); and Acetyl-CoA Carboxylase (ACC) (ACC—Enzyme Linker—cTPR6 Spacer—ID Linker—ID17) (SEQ ID NO:178).

FIG. 13D contains the amino acid sequences of the Cannabinoidergic Metabolon Scaffold (CBSCFLD)—(Myc)₃ (SEQ ID NO:179) and the Malonyl-CoA Metabolon Scaffold (MCASCFLD)—(FLAG)₃ (SEQ ID NO:180).

FIG. 14A contains codon-optimized nucleotide sequences (SEQ ID NOs:181-187) encoding the enzymes of FIG. 13A.

FIG. 14B contains the codon-optimized nucleotide sequences (SEQ ID NOs:188-194) encoding the enzymes of FIG. 13B.

FIG. 14C contains the codon-optimized nucleotide sequences (SEQ ID NOs:195-200) encoding the enzymes of FIG. 13C.

FIG. 14D contains the codon-optimized nucleotide sequences (SEQ ID NO:201 and SEQ ID NO:202) encoding the scaffolds of FIG. 13D.

FIG. 15A contains the nucleotide sequence of the HCA gene cassette (SEQ ID NO:203).

FIG. 15B contains the nucleotide sequence of the GPP gene cassette (SEQ ID NO:204).

FIG. 15C contains the nucleotide sequence of the CAN gene cassette (SEQ ID NO:205).

FIG. 15D contains the nucleotide sequence of the SCF gene cassette (SEQ ID NO:206).

FIG. 15E contains the nucleotide sequence of the SOL gene cassette (SEQ ID NO:207).

FIG. 16 is a map of the pCCI-Brick plasmid construct.

FIG. 17 is a map of a pESC-TRP (“vHCA”) vector construct. In this map, the vector contains a TRP gene allowing selection in tryptophan deficient media. Similar vectors also were made in which the TRP gene was replaced with a LEU gene allowing selection in leucine deficient media, a HIS3 gene allowing selection in histidine deficient media, or a URA3 gene allowing selection in uracil deficient media.

FIG. 18 is a graph of the proliferation curves for yCBSCF and yCBSOL cultures. Line plots depicting cell proliferation curves were fitted via nonlinear regression of cell density measurements (OD₆₀₀ₙₘ) recorded in 12-hour intervals over a 48-hour incubation period for yCBSCF and yCBSOL cultures. Initial cell densities for all cultures were standardized to OD₆₀₀ₙₘ=0.3. For all measures, n=3 biological replicates for yCBSCF and yCBSOL cultures. Floating data points depict means with 95% confidence intervals. Dotted lines represent 95% confidence intervals for regression curve fits.

FIGS. 19A-19E show a comparison of cannabinoid and precursor titers for scaffolded and soluble cannabinoid biosynthesis. Representative mass spectra of target analytes isolated from (FIG. 19A) yCBSOL and (FIG. 19B) yCBSCF cultures incubated for 48 hours in basal culture media. Bar plots depicting (FIG. 19C) Total (aggregate) cannabinoid (CBGA+CBDA+CBCA+CBG+CBD+CBC) titers, (FIG. 19D) cannabinoid precursor (OVA) titers and summated parent and decarboxylation derivative (CBGA+CBG, CBDA+CBD, and CBCA+CBC) cannabinoid titers, and (FIG. 19E) separated parent (COO(H)) cannabinoid (CBGA, CBDA, and CBCA) and decarboxylation derivative (ΔCOOH) cannabinoid (CBG, CBD, and CBC) titers for 48-hour yCBSOL (left) and yCBSCF (right) cultures grown in basal culture media. For all measures, n=3 biological replicates for yCBSCF and yCBSOL cultures. CB, cannabinoid; Cannabigerolic acid, CBGA; cannabigerol, CBG; cannabidiolic acid, CBDA; cannabidiol, CBD; cannabichromenic acid, CBCA; cannabichromene, CBC; olivetolic acid, OVA. Floating asterisks indicate statistically significant (determined by Bonferroni's multiple comparisons post-hoc test; α=0.05) between-strain differences for yCBSCF versus yCBSOL cultures. Bar plots depict means with 95% confidence intervals. *p<0.05; **p<0.01; ***p<0.001; ****p<0.0001.

FIG. 20 is a bar plot of the impact of citrate and hexanoate supplementation on scaffolded and soluble cannabinoid biosynthesis. Total cannabinoid (CBGA+CBDA+CBCA+CBG+CBD+CBC) titers are shown for yCBSOL and yCBSCF cultures incubated for 48 hours in basal, hexanoate (300 mg/L)-supplemented, and buffered (pH 6.0) citrate (300 mg/L)-supplemented culture media. Floating asterisks indicate statistically significant (determined by Bonferroni's multiple comparisons post-hoc test; α=0.05) between-strain differences for yCBSCF versus yCBSOL cultures. Lines with asterisks indicate statistically significant (determined by Bonferroni's multiple comparisons post-hoc test; α=0.05) within-strain differences for basal media total cannabinoid titers versus citrate-supplemented media total cannabinoid titers for yCBSCF cultures. Bar plots depict means with 95% confidence intervals. *p<0.05; **p<0.01; ***p<0.001; ****p<0.0001.

FIGS. 21A and 21B show concentration-response parameterization of scaffolded and soluble cannabinoid biosynthesis from citrate. In FIG. 21A, line plots are shown depicting eight-point concentration ([citrate])—response (total cannabinoid titers) curves fitted via asymmetric sigmoidal (five-parameter) logistic regression and in FIG. 21B, bar graphs are shown depicting concentration-response parameter estimates (CB_{Max}, the estimated maximum total cannabinoid titers and citrate EC₅₀, the estimated citrate concentration yielding half-maximal total cannabinoid titers) for 48-hour yCB_{SCF} and yCB_{SOL} cultures incubated for 48 hours in culture media supplemented with 0, 10, 30, 100, 300, 1000, 3000, or 10000 mg/L buffered (pH 6.0) citrate. For all measures, n=3 biological replicates for yCB_{SCF} and yCB_{SOL} cultures. Floating asterisks indicate statistically significant (determined by Bonferroni's multiple comparisons post-hoc test; α=0.05) between-strain differences for yCB_{SCF} versus yCB_{SOL} cultures. Floating data points and bar plots depict means with 95% confidence intervals. Dotted lines represent 95% confidence intervals for regression curve fits. *p<0.05; **p<0.01; ***p<0.001; ****p<0.0001.

### DETAILED DESCRIPTION

This document provides methods and materials for producing cannabinoids in host cells or in vitro using a bidirectional, multi-enzymatic scaffold, which can control the localization and stoichiometry of enzymes catalyzing the biosynthesis of cannabinoids and cannabinoid precursors. As described herein, one or more cannabinoids including cannabigerolic acid (CBGA), cannabidiolic acid (CBDA), cannabichromenic acid (CBCA), and tetrahydrocannabinolic acid, can be produced using a bidirectional, multi-enzymatic scaffold and one or more soluble cannabinoid synthesis enzymes, and the conjugate bases, cannabigerolate, cannabidiolate, cannabichromenate, and tetrahydrocannabinolate, respectively, and decarboxylation products, cannabigerol (CBG), cannabidiol (CBD), cannabichromene (CBC), and tetrahydrocannabinol, respectively, of these cannabinoids also can be produced, as can the tetrahydrocannabinolic acid oxidation product cannabinolic acid and its decararboxylation product cannabinol. The bidirectional, multi-enzymatic scaffold described herein results in significant increases in cannabinoid production in recombinant hosts, including total cannabinoid, CBGA, CBG, CBDA, CBD, CBCA, CBC, and olivetolic acid precursor production, as compared with cannabinoid production in recombinant hosts using the same enzymes that are not bound to a scaffold. As used herein, enzymes that are not bound to a scaffold are referred to as soluble or non-scaffolded. While one particular form of a cannabinoid or other compound may be referenced herein, it is understood that any of its neutral or ionized forms, including any salt forms thereof or decarboxylation derivatives thereof (e.g., produced in the presence of heat and light), are included unless otherwise indicated. It is understood by those skilled in the art that the specific form will depend on factors such as pH and carboxylation status.

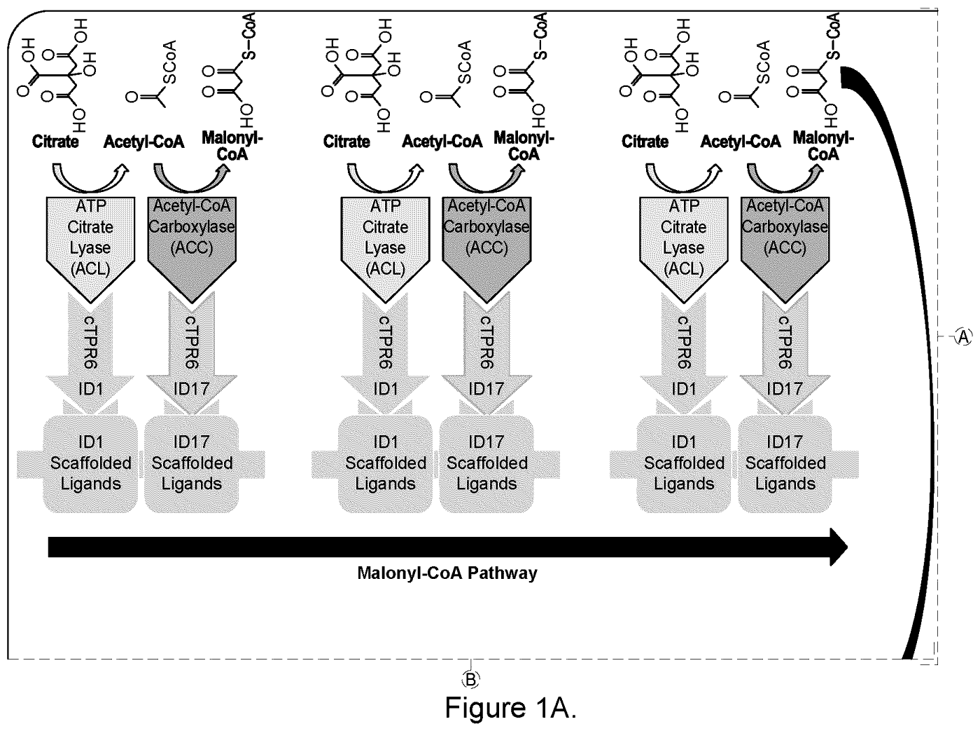

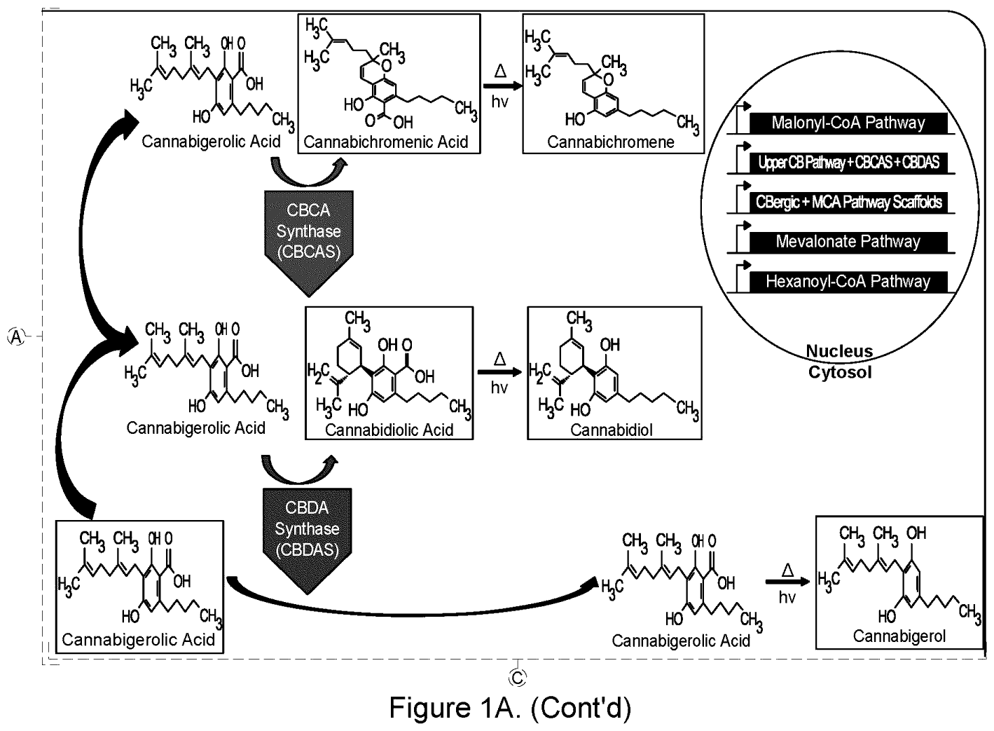

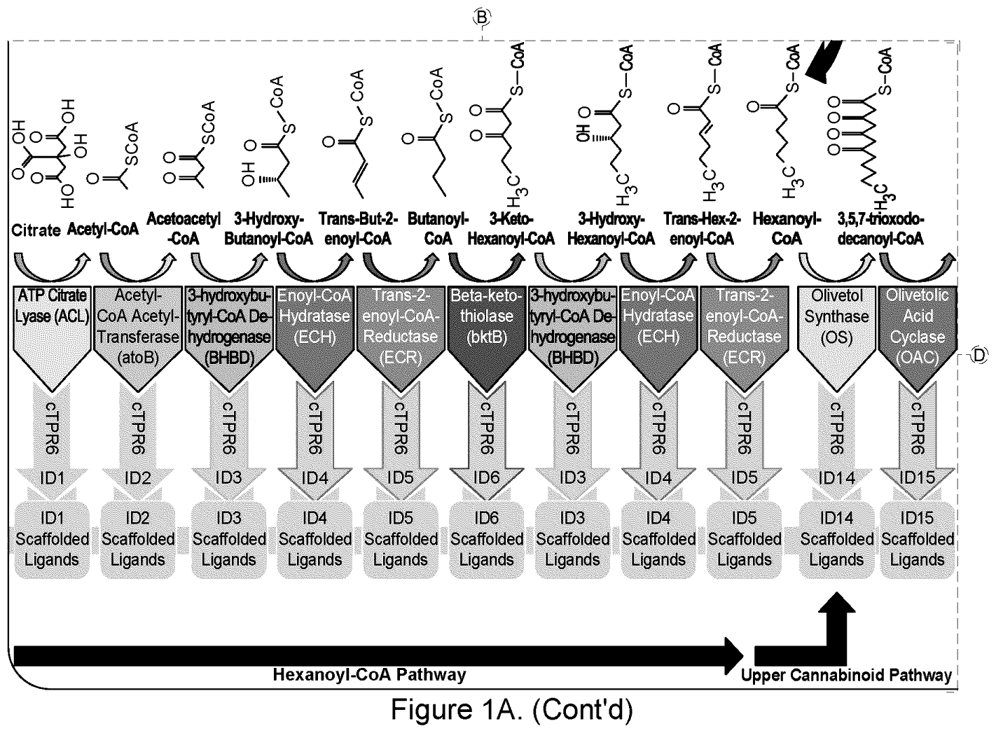

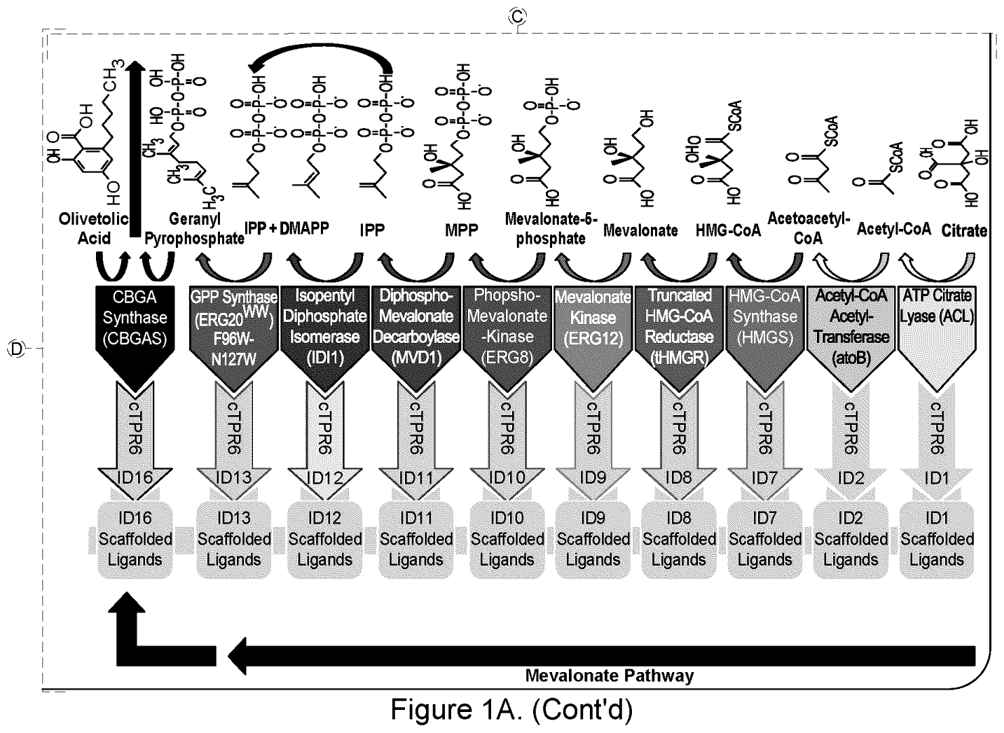

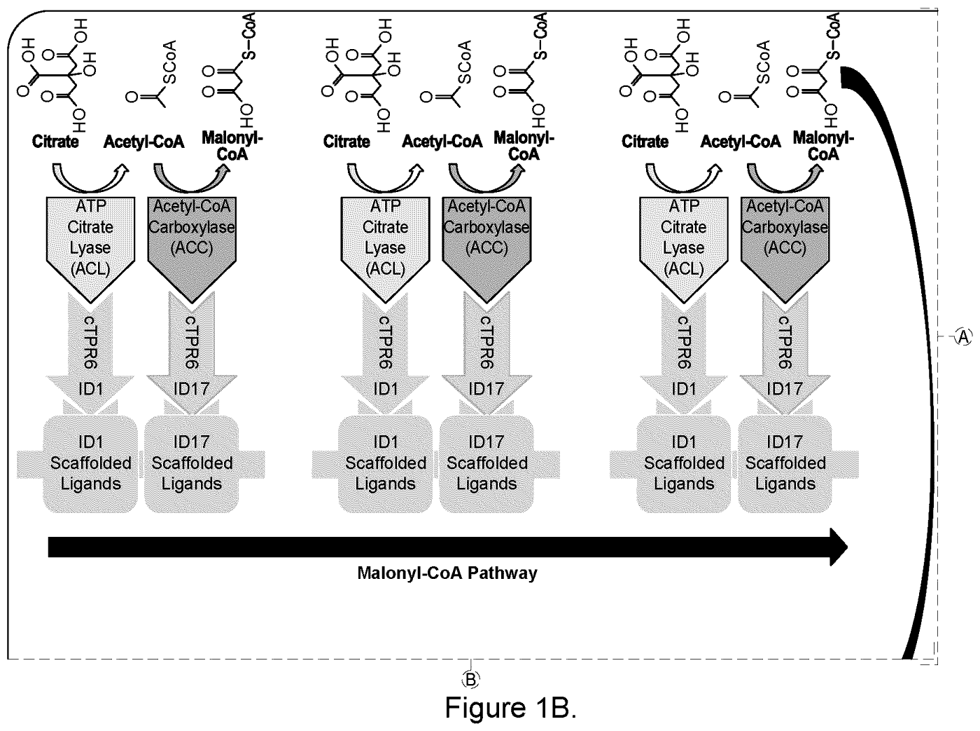

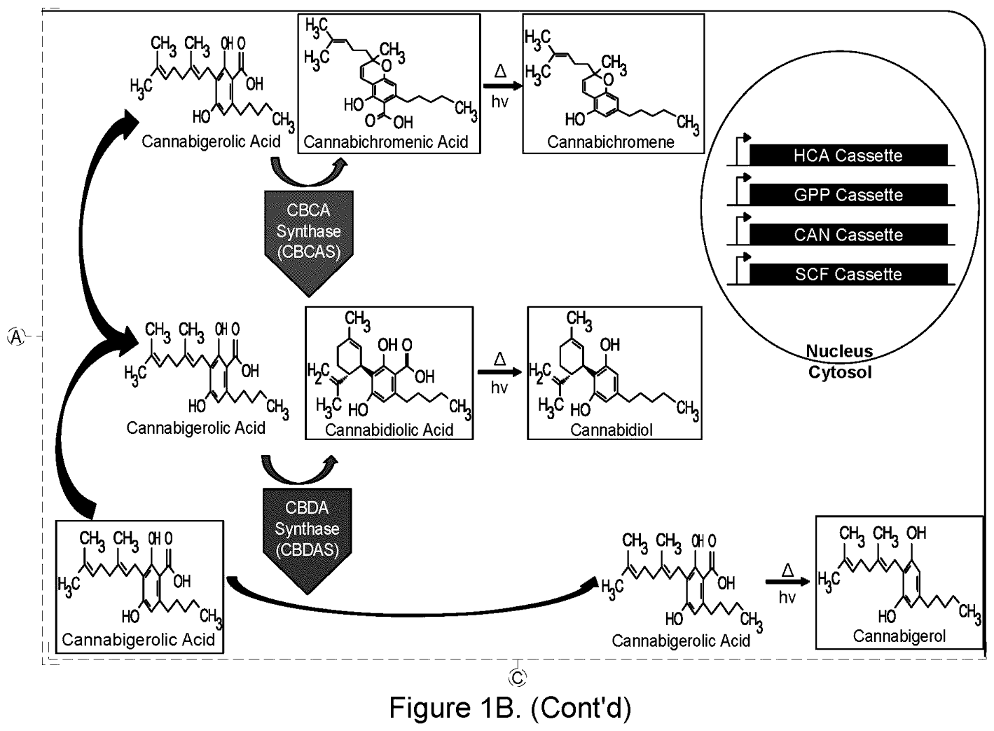

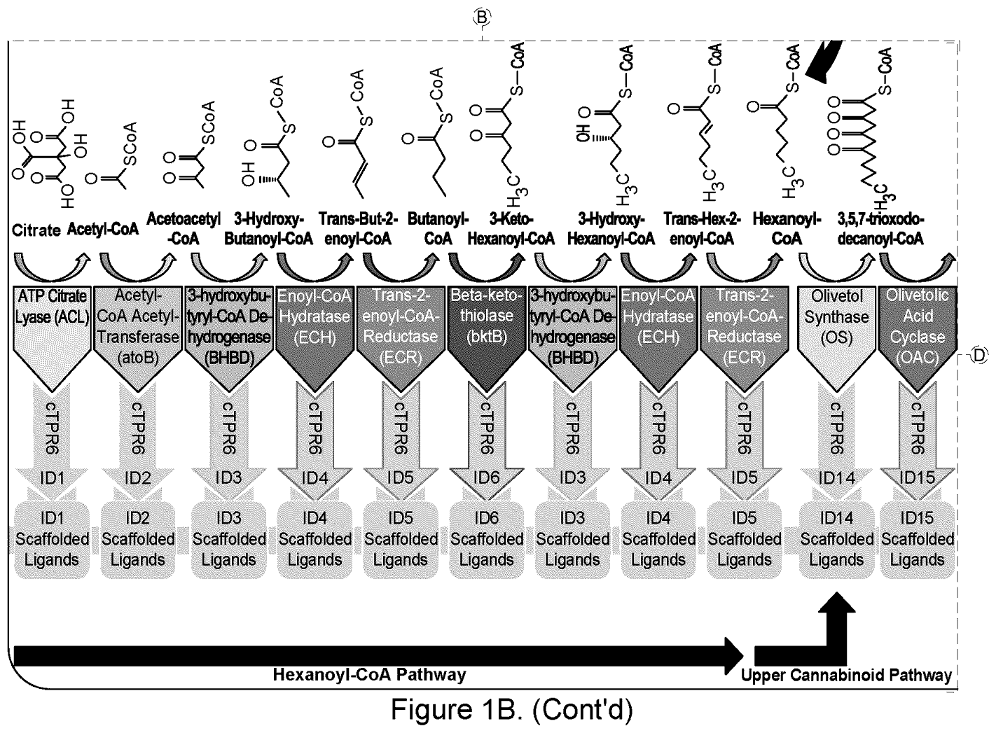

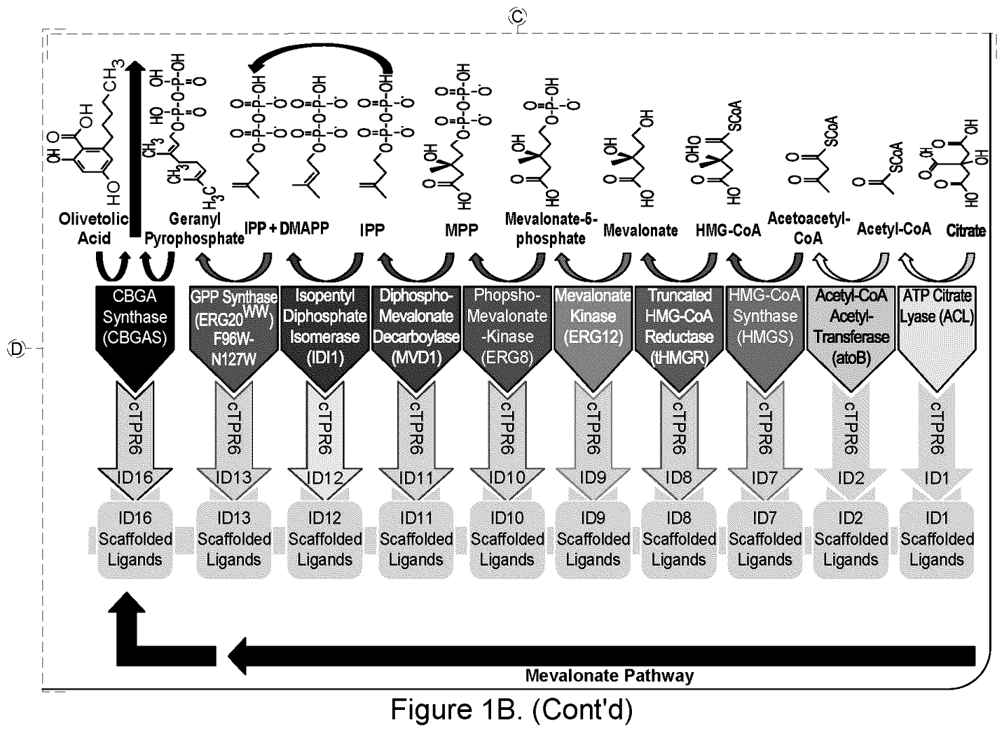

In general, enzymes described herein, which can be co-localized on one or more scaffolds and used for producing cannabinoids or cannabinoid precursors, are engineered to contain an interaction domain (ID), which can be separated from the enzyme by an amino acid spacer sequence at the N- or C-terminus of the enzyme. The ID can be composed of two or more scaffold-binding motifs. The engineered enzymes also can include one or more linkers between the enzyme, spacer, and/or ID. The engineered enzymes can bind to a scaffold, which is a polypeptide that contains unique ID-binding domains, i.e., tandem peptide ligands, as shown in FIG. 1A and FIG. 1B, such that the enzymes are co-localized to the scaffold. In other words, each enzyme can be engineered to contain a protein-protein interaction domain that is specific for ligand or ligands (binding site) on the scaffold such that the enzyme can be localized to a discrete location along the scaffold via non-covalent interactions. In some cases, the engineered enzymes can be chimeric enzymes. The scaffolded ligands can be separated using amino acid linkers or spacers. See, for example, Horn and Sticht, Frontiers in Bioengineering and Biotechnology, 2015, volume 3, article 191; Whitaker and Dueber, Methods in Enzymology, Chapter 19, “Metabolic Pathway Flux Enhancement by Synthetic Protein Scaffolding,” Volume 497, 2011, for descriptions of IDs, binding domains, linkers and spacers. IDs also can be referred to as adaptor domains.

Typically, each interaction domain consists of two tandem scaffold-binding motifs that continue/extend from the C-terminus of the engineered enzyme and that can bind to their corresponding scaffolded peptide ligands, which are constructed in tandem along the scaffold. Dual-binding of enzymes to the scaffold ensures fixed spatial orientation, increases binding specificity for each ID-scaffold interaction, and better tethers each enzyme to the scaffold, all of which can improve pathway flux by enabling substrate channeling through each enzymatic step in the scaffolded biosynthetic pathways.

In some embodiments, there are more than two, e.g., three, four, five, six, seven, eight, nine, or ten, or more molecules of each enzyme localized to the scaffold. In addition, the ratio of any given enzyme in a biosynthetic pathway to any other enzyme in the biosynthetic pathway can be varied. For example, the ratio of one engineered enzyme in a pathway to a second engineered enzyme in the same pathway can be varied, e.g., from about 1:5 to about 5:1, e.g., from about 1:5 to about 2:5, from about 2:5 to about 3:5, from about 3:5 to about 5:5, from about 5:5 to about 5:3, from about 5:3 to about 5:2, or from about 5:2 to about 5:1.

The peptide ligands are typically short peptide sequences, ranging in length from 3 to 50 amino acid residues. For example, a peptide ligand can be 3-10, 7-15, 10-20, 15-25, 20-30, 25-35, 30-40, 35-45, or 40-50 amino acids in length. There is a database of over 200 different motifs available on the web at elm.eu.org that can be used as described herein. See, for example, Dinkel et al., Nucleic Acids Res. 2014; 42 (Database issue): D259-D266.

An ID can be a peptide sequence ranging in length 3 to 200 amino acid residues. For example, the ID can be 3-10, 7-15, 10-20, 15-25, 20-30, 25-35, 30-40, 35-45, 40-50, 45-55, 50-60, 65-75, 70-80, 85-95, 90-100, 100-110, 105-115, 110-120, 115-125, 120-130, 125-135, 130-140, 135-145, 140-150, 135-145, 140-150, 145-155, 150-160, 165-175, 170-180, 175-185, 180-190, 185-195, or 190-200 amino acids in length. For example, an ID can be a SH2 domain, a SH3 domain, a PDZ domain, a GTPase binding domain (GBD), a leucine zipper domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, a fibronectin receptor domain, a FNDC domain, a SAMD domain, a WBP domain, and/or a SASH domain. See, e.g., U.S. Pat. No. 9,856,460 for a list of domains that can be uses as an ID as described herein.

For example, an ID can be a “Src homology2” (SH2) or a “Src homology3” (SH3) domain. SH2 domains are highly conserved structures of approximately 100 amino acid residues that comprise two α-helices and seven β-strands. The SH2 domain can have a promiscuous or strict specificity for a 3-5 amino acid motif flanking a phosphorylated tyrosine. See, Horn and Sticht, 2015, supra. For example, a SH2 domain that can be used as an ID as described herein can be residues 5-122 of a mouse Ct10 regulator of kinase adaptor (Crk) protein having GenBank Accession No. AAH31149.

SH3 domains are small modules of approximately 60 residues that bind proline-rich ligands, which bind to the domain surface at three shallow grooves formed by conserved aromatic residues and exhibit two different binding orientations. See, Horn and Sticht, 2015, supra. In some embodiments, the proline-rich ligand can have a core PXXP motif flanked by a positively charged residue. Class I PZP domains recognize ligands conforming to the consensus +XXPXXP (where+is either Arg or Lys), while Class II domains recognize PXXPX+ motifs and bind to ligands in the opposite orientation. See, Teyra, et al., FEBS Lett., 2012 586(17):2631-7. Individual SH3 domains do not measurably interact with other SH3 domain family ligands within an organism, minimizing cross-talk and increasing the number of domain/ligand pairs available for simultaneous use. See, Whitaker and Dueber, 2011, supra. For example, a SH3 domain that can be used as an ID as described herein can be residues 134-190 of a mouse Crk protein having GenBank Accession No. AAH31149 and its peptide ligand can be PPPALPPKRRR (SEQ ID NO:1).

For example, an ID can be a PDZ (PSD-95/Discs-large/ZO1) domain. PDZ domains are approximately 100 amino acid residues in length and target specific motifs at the C-terminus of the binding partner. The peptide ligand adopts a β-strand and extends an existing β-sheet within the PDZ domain upon binding. At least four different classes of ligands are known for PDZ domains exhibiting a distinct binding specificity. See, Horn and Sticht, 2015, supra. For example, grouped PDZ domains into two main specificity classes based on distinct ligand signatures: Class I PDZ domains recognize a (X[T/S]XϕCOOH) motif, Class II PDZ domains recognize a (XϕXϕCOOH) motif, and Class III PDZ domains recognize a X[ED]XϕCOOH motif, where X is any residue and ϕ is a hydrophobic amino acid. See, Teyra, et al., 2012, supra. PDZ and SH3 domains are found throughout eukaryotic and eubacterial genomes. For example, a PDZ domain that can be used as an ID as described herein can be residues 77-171 of a mouse α-syntrophin protein having GenBank Accession No. EDL06069 and the peptide ligand can be GVKESLV (SEQ ID NO:208).

For example, an ID can be a GBD domain from a protein such as the Wiskott-Aldrich syndrome-like protein (N-WASP). Isolated GBD domains do not adopt a single, discrete structure under physiological conditions but rather exhibit multiple, loosely packed conformations in solution. The corresponding peptide ligand has been deduced from the autoinhibited form of the GBD. See, Horn and Sticht, 2015, supra. For example, a GBD domain that can be used as an ID described herein can include residues 196 to 274 of a rat N-WASP protein having GenBank Accession No. BAA21534, and its peptide ligand, which can be LVGALMHVMQKRSRAIHSSDEGEDQAGDEDED (SEQ ID NO:2), can be used as a peptide ligand as described herein.

For example, an ID can have a leucine zipper or synthetic coiled-coil domain. A leucine zipper domain can include multiple interspersed leucine residues approximately seven amino acid residues apart. Havranek, and Harbury ((2003), Nat. Struct. Biol. 10, 45-52) identified new pairs of homodimers or heterodimers by altering residues between leucine zipper pairs based on computational prediction. Reinke, et al. ((2010). J. Am. Chem. Soc. 132, 6025-6031) identified three pairs of synthetic coiled coils that do not exhibit measurable self-association. See, Whitaker and Dueber, 2011, supra. One example of an ID that can be used as described herein can be ITIRAAFLEKENTALRTEIAELEKEVGRCENIVSKYETRYGPL (SEQ ID NO:3), and its peptide ligand for use as described herein can be LEIRAAFLEKENTALRTRAAELRKRVGRCRNIVSKYETRYGPL (SEQ ID NO:4).

For example, an ID can be a dockerin polypeptide, which can localize to a specific cohesion polypeptide on a scaffold described herein. Cohesion-dockerin pairs are particularly useful for ex vivo applications as binding is calcium dependent. See, Whitaker and Dueber, 2011, supra.

Combinations of IDs that have high affinity for their peptide ligands and high specificity, i.e., minimal cross-reactivity, can be used as described herein to allow for binding of multiple, different enzymes to a scaffold provided herein. For example, at least three different enzymes can be localized on a scaffold. In some embodiments, at least four different enzymes can be localized on a scaffold. In some embodiments, at least five different enzymes can be localized on a scaffold. In some embodiments, at least six different enzymes can be localized on a scaffold. In some embodiments, at least seven different enzymes can be localized on a scaffold. In some embodiments, at least eight different enzymes can be localized on a scaffold. In some embodiments, at least nine different enzymes can be localized on a scaffold. In some embodiments, at least ten different enzymes can be localized on a scaffold. In some embodiments, at least eleven different enzymes can be localized on a scaffold. In some embodiments, at least twelve different enzymes can be localized on a scaffold. In some embodiments, at least fifteen different enzymes can be localized on a scaffold. In some embodiments, at least seventeen different enzymes can be localized on a scaffold. In some embodiments, at least eighteen different enzymes can be localized on a scaffold. In some embodiments, at least twenty different enzymes can be localized on a scaffold. In some embodiments, at least twenty-one different enzymes can be localized on a scaffold.

Table 1 provide exemplary combinations of heterologous IDs, i.e., IDs that are different from each other, that can be used in seventeen different engineered enzymes and Table 2 provides the corresponding exemplary combinations of peptide ligands that can be used to localize the seventeen different enzymes to one or more scaffolds. In the embodiments shown in Tables 1 and 2, each ID is composed of two tandem peptide motifs as are the corresponding peptide ligands, which interact with the tandem peptide motifs. It will be appreciated that any one of the enzymes listed in Tables 1 and 2 can be used in combination with any of the listed combinations of IDs and corresponding peptide ligands.

TABLE 1
Interaction Domain Motif Sequences in Engineered Enzymes

| | ID | ID | | |
|---|---|---|---|---|
| Enzyme | # | Motif # | ID Motif | ID Motif Amino Acid Sequence |
| ATP Citrate Lyase | 1 | 1 | SYNZIP1 | SYYHHHHHHLESTSLYKKAGSG |
| | | | | SNLVAQLENEVASLENENETLK |
| | | | | KKNLHKKDLIAYLEKEIANLRK |
| | | | | KIEE ((SEQ ID NO: 5)) |
| | | 2 | SYNZIP2 | SYYHHHHHHLESTSLYKKAGSG |
| | | | | SARNAYLRKKIARLKKDNLQLE |
| | | | | RDEQNLEKIIANLRDEIARLENE |
| | | | | VASHEQ (SEQ ID NO: 6) |
| Acetyl-CoA | 2 | 1 | SYNZIP3 | SYYHHHHHHLESTSLYKKAGSG |
| Acetyltransferase | | | | SNEVTTLENDAAFIENENAYLE |
| (atoB) | | | | KEIARLRKEKAALRNRLAHKK |
| | | | | (SEQ ID NO: 7) |
| | | 2 | SYNZIP4 | SYYHHHHHHLESTSLYKKAGSG |
| | | | | SQKVAELKNRVAVKLNRNEQL |
| | | | | KNKVEELKNRNAYLKNELATLE |
| | | | | NEVARLENDVAE (SEQ ID NO: 8) |
| 3-hydroxybutyryl-CoA | 3 | 1 | MYND | ENLYFQGENLYFQGDSSESCWN |
| Dehydrogenase | | | | CGRKASETCSGCNTARYCGSFC |
| | | | | QHKDWEKHHHICGQTLQAQQ |
| | | | | (SEQ ID NO: 9) |
| | | 2 | UEV | MAVSESQLKKMVSKYKYRDLT |
| | | | | VRETVNVITLYKDLKPVLDSYV |
| | | | | FNDGSSRELMNLTGTIPVPYRG |
| | | | | NTYNIPICLWLLDTYPYNPPICF |
| | | | | VKPTSSMTIKTGKHVDANGKIY |
| | | | | LPYLHEWKHPQSDLLGLIQVMI |
| | | | | VVFGDEPPVFSRP |
| | | | | (SEQ ID NO: 10) |
| Enoyl-CoA Hydratase | 4 | 1 | PABP | GPLGSPLTASMLASAPPQEQKQ |
| | | | | MLGERLFPLIQAMHPTLAGKITG |
| | | | | MLLEIDNSELLHMLESPESLRSK |
| | | | | VDEAVAVLQAHQAKEAAQKA |
| | | | | (SEQ ID NO: 11) |
| | | 2 | MDM2 | NTNMSVPTDGAVTTSQIPASEQ |
| | | | | ETLVRPKPLLLKLLKSVGAQKD |
| | | | | TYTMKEVLFYLGQYIMTKRLYD |
| | | | | EKQQHIVYCSNDLLGDLFGVPS |
| | | | | FSVKEHRKIYTMIYRNLVV (SEQ |
| | | | | ID NO: 12) |
| Trans-Enoyl-CoA | 5 | 1 | SYNZIP10 | SYYHHHHHHLESTSLYKKAGSG |
| Reductase | | | | SNLLATLRSTAAVLENENHVLE |
| | | | | KEKEKLRKEKEQLLNKLEAYK |
| | | | | (SEQ ID NO: 13) |
| | | 2 | SYNZIP22 | SYYHHHHHHLESTSLYKKAGSG |
| | | | | SKRIAYLRKKIAALKKDNANLE |
| | | | | KDIANLENEIERLIKEIKTLENEV |
| | | | | ASHEQ (SEQ ID NO: 14) |
| Beta-ketothiolase | 6 | 1 | GYF | DVMWEYKWENTGDAELYGPFT |
| (bktB) | | | | SAQMQTWVSEGYFPDGVYCRK |
| | | | | LDPPGGQFYNSKRIDFDLYT |
| | | | | (SEQ ID NO: 15) |
| | | 2 | PAH | ESDSVEFNNAISYVNKIKTRFLD |
| | | | | HPEIYRSFLEILHTYQKEQLHTK |
| | | | | GRPFRGMSEEEVFTEVANLFRG |
| | | | | QEDLLSEFGQFLPEAKR (SEQ ID |
| | | | | NO: 16) |
| HMG-COA Synthase | 7 | 1 | WW1A | LGPLPPGWEVRSTVSGRIYFVD |
| | | | | HNNRTTQFTDPRLH (SEQ ID |
| | | | | NO: 17) |
| | | 2 | WW1B | GAMGPLPPGWEKRTDSNGRVY |
| | | | | FVNHNTRITQWEDPRS (SEQ ID |
| | | | | NO: 18) |
| HMG-COA Reductase | 8 | 1 | FOS | SYYHHHHHHLESTSLYKKAGSE |
| | | | | FFRRERNKMAAAKCRNRRRELT |
| | | | | DTLQAETDQLEDEKSALQTEIA |
| | | | | NLLKEKEKLEFILAAHRPACKIP |
| | | | | DDLGFPEEMSLE (SEQ ID |
| | | | | NO: 19) |
| | | 2 | SYNZIP9 | SYYHHHHHHLESTSLYKKAGSG |
| | | | | SQKVESLKQKIEELKQRKAQLK |
| | | | | NDIANLEKEIAYAET (SEQ ID |
| | | | | NO: 20) |
| Mevalonate Kinase | 9 | 1 | VHS1 | MEPAMEPETLEARINRATNPLN |
| | | | | KELDWASINGFCEQLNEDFEGP |
| | | | | PLATRLLAHKIQSPQEWEAIQAL |
| | | | | TVLETCMKSCGKRFHDEVGKFR |
| | | | | FLNELIKVVSPKYLGSRTSEKVK |
| | | | | NKILELLYSWTVGLPEEVKIAEA |
| | | | | YQMLKKQGIVKS (SEQ ID |
| | | | | NO: 21) |
| | | 2 | VHS2 | GAMGSMAEAEGESLESWLNKA |
| | | | | TNPSNRQEDWEYIIGFCDQINKE |
| | | | | LEGPQIAVRLLAHKIQSPQEWEA |
| | | | | LQALTVLEACMKNCGRRFHNE |
| | | | | VGKFRFLNELIKVVSPKYLGDR |
| | | | | VSEKVKTKVIELLYSWTMALPE |
| | | | | EAKIKDAYHMLKRQGIVQSDPPI |
| | | | | PVDRTLIPSPPPRPKN (SEQ ID |
| | | | | NO: 22) |
| Phosphomevalonate | 10 | 1 | SYNZIP13 | SYYHHHHHHLESTSLYKKAGSG |
| Kinase | | | | SQKVEELKNKIAELENRNAVKK |
| | | | | NRVAHLKQEIA YLKDELAAHEF |
| | | | | E (SEQ ID NO: 23) |
| | | 2 | SYNZIP15 | SYYHHHHHHLESTSLYKKAGSG |
| | | | | SFENVTHEFILATLENENAKLRR |
| | | | | LEAKLERELARLRNEVAWL |
| | | | | (SEQ ID NO: 24) |
| Diphosphomevalonate | 11 | 1 | MATH | AMADLEQKVLEMEASTYDGVFI |
| Decarboxylase | | | | WKISDFPRKRQEAVAGRIPAIFS |
| | | | | PAFYTSRYGYKMCLRIYLNGDG |
| | | | | TGRGTHLSLFFVVMKGPNDALL |
| | | | | RWPFNQKVTLMLLDQNNREHV |
| | | | | IDAFRPDVTSSSFQRPVNDMNIA |
| | | 2 | SKP1 | SGCPLFCPVSKMEAKNSYVRDD |
| | | | | AIFIKAIVDLTGL (SEQ ID NO: 25) |
| | | | | ASIKLQSSDGEIFEVDVEIAKQS |
| | | | | VTIKTMLEDLGMDDEGDDDPVP |
| | | | | LPNVNAAILKKVIQWCTHHKDD |
| | | | | PPPPEDDENKEKRTDDIPVWDQ |
| | | | | EFLKVDQGTLFELILAANYLDIK |
| | | | | GLLDVTCKTVANMIKGKTPEEI |
| | | | | RKTFNIKNDFTEEEEAQVRKEN |
| | | | | QWC (SEQ ID NO: 26) |
| Isopentenyl- | 12 | 1 | SYNZIP5 | SYYHHHHHHLESTSLYKKAGSG |
| Diphosphate Delta- | | | | SNTVKELKNYIQELEERNAELK |
| Isomerase | | | | NLKEHLKFAKAELEFELAAHKF |
| | | | | E (SEQ ID NO: 27) |
| | | 2 | SYNZIP6 | SYYHHHHHHLESTSLYKKAGSG |
| | | | | SQKVAQLKNRVAYKLKENAKL |
| | | | | ENIVARLENDNANLEKDIANLE |
| | | | | KDIANLERDVAR (SEQ ID |
| | | | | NO: 28) |
| Geranyl-Diphosphate | 13 | 1 | PDZ1 | LCTMKKGPSGYGFNLHSDKSKP |
| Synthase | | | | GQFIRSVDPDSPAEASGLRAQDR |
| | | | | IVEVNGVCMEGKQHGDVVSAIR |
| | | | | AGGDETKLLVVDRE (SEQ ID |
| | | | | NO: 29) |
| | | 2 | PDZ2 | SSGAIIYTVELKRYGGPLGITISG |
| | | | | TEEPFDPIIISSLTKGGLAERTGAI |
| | | | | HIGDRILAINSSSLKGKPLSEAIH |
| | | | | LLQMAGETVTLKIKKQTDAQPA |
| | | | | SS (SEQ ID NO: 30) |
| Olivetol Synthase | 14 | 1 | SH2A | GNNLETYEWYNKSISRDKAEKL |
| | | | | LLDTGKEGAFMVRDSRTPGTYT |
| | | | | VSVFTKAIISENPCIKHYHIKETN |
| | | | | DSPKRYYVAEKYVFDSIPLLIQY |
| | | | | HQYNGGGLVTRLRYPVCG (SEQ |
| | | | | ID NO: 31) |
| | | 2 | SH2B | GSHPWFFGKIPRAKAEEMLSKQ |
| | | | | RHDGAFLIRESESAPGDFSLSVK |
| | | | | FGNDVQHFKVLRDGAGKYFLW |
| | | | | VVKFNSLNELVDYHRSTSVSRN |
| | | | | QQIFLRDIEQVPQQPT (SEQ ID |
| | | | | NO: 32) |
| Olivetolic Acid | 15 | 1 | PTB1 | GQDRSEATLIKRFKGEGVRYKA |
| Cyclase | | | | KLIGIDEVSAARGDKLCQDSMM |
| | | | | KLKGVVAGARSKGEHKQKIFLT |
| | | | | ISFGGIKIFDEKTGALQHHHAVH |
| | | | | EISYIAKDITDHRAFGYVCGKEG |
| | | | | NHRFVAIKTAQAAEPVILDLRDL |
| | | | | FQLIYELKQREELEKKA (SEQ ID |
| | | | | NO: 33) |
| | | 2 | PTB2 | GSHMGSQFWVTSQKTEASERC |
| | | | | GLQGSYILRVEAEKLTLLTLGA |
| | | | | QSQILEPLLFWPYTLLRRYGRDK |
| | | | | VMFSFEAGRRCPSGPGTFTFQTS |
| | | | | QGNDIFQAVEAAIQQQKAQGKV |
| | | | | GQAQDILRLEHHHHHH (SEQ ID |
| | | | | NO: 210) |
| CBGA Synthase | 16 | 1 | SH3A | AEYVRALFDFNGNDEEDLPFKK |
| | | | | GDILRIRDKPEEQWWNAEDSEG |
| | | | | KRGMIPVPYVEKY (SEQ ID |
| | | | | NO: 34) |
| | | 2 | SH3B | LIKHMRAEALFDFTGNSKLELN |
| | | | | FKAGDVIFLLSRINKDWLEGTV |
| | | | | RGATGIFPLSFVKILK (SEQ ID |
| | | | | NO: 35) |
| Acetyl-CoA | 17 | 1 | FAT | GSHMRLGAQSIQPTANLDRTDD |
| | | | | LVYLNVMELVRAVLELKNELA |
| | | | | QLPPEGYVVVVKNVGLTLRKLI |
| | | | | GSVDDLLPSLPSSSRTEIEGTQKL |
| | | | | LNKDLAELINKMRLAQQNAVTS |
| | | | | LSEECKRQMLTASHTLAVDAKN |
| | | | | LLDAVDQAKVLANLAHPPAE |
| | | | | (SEQ ID NO: 36) |
| Carboxylase | | 2 | PEX | GAMATPGSENVLPREPLIATAV |
| | | | | KFLQNSRVRQSPLATRRAFLKK |
| | | | | KGLTDEEIDMAFQQSGTAADEP |
| | | | | SSLW (SEQ ID NO: 37) |

TABLE 2
Tandem Peptide Ligand Sequences in Scaffold

| | ID | ID Motif | | ID Motif Scaffolded Ligand |
|---|---|---|---|---|
| Enzyme | # | # | ID Motif | Amino Acid Sequence |
| ATP Citrate Lyase | 1 | 1 | SYNZIP1 | SYYHHHHHHLESTSLYKKAGS |
| | | | | GSARNAYLRKKIARLKKDNLQ |
| | | | | LERDEQNLEKIIANLRDEIARLE |
| | | | | NEVASHEQ (SEQ ID NO: 6) |
| | | 2 | SYNZIP2 | SYYHHHHHHLESTSLYKKAGS |
| | | | | GSNLVAQLENEVASLENENETL |
| | | | | KKKNLHKKDLIA YLEKEIANLR |
| | | | | KKIEE (SEQ ID NO: 5) |
| Acetyl-CoA | 2 | 1 | SYNZIP3 | SYYHHHHHHLESTSLYKKAGS |
| Acetyltransferase | | | | GSQKVAELKNRVAVKLNRNEQ |
| (atoB) | | | | LKNKVEELKNRNAYLKNELAT |
| | | | | LENEVARLENDVAE (SEQ ID |
| | | | | NO: 8) |
| | | 2 | SYNZIP4 | SYYHHHHHHLESTSLYKKAGS |
| | | | | GSNEVTTLENDAAFIENENAYL |
| | | | | EKEIARLRKEKAALRNRLAHK |
| | | | | K (SEQ ID NO: 7) |
| 3-hydroxybutyryl- | 3 | 1 | MYND | RPPTISNPPPLISSAKHPSV (SEQ |
| CoA Dehydrogenase | | | | ID NO: 38) |
| | | 2 | UEV | NFLQSRPEPTAPPEESFRSG |
| | | | | (SEQ ID NO: 39) |
| Enoyl-CoA Hydratase | 4 | 1 | PABP | SKGTGLNPNAKVWQEIAPGN |
| | | | | (SEQ ID NO: 40) |
| | | 2 | MDM2 | PDGGTTFEHLWSSLEPDSTY |
| | | | | (SEQ ID NO: 41) |
| Trans-Enoyl-CoA | 5 | 1 | SYNZIP10 | SYYHHHHHHLESTSLYKKAGS |
| Reductase | | | | GSKRIAYLRKKIAALKKDNAN |
| | | | | LEKDIANLENEIERLIKEIKTLE |
| | | | | NEVASHEQ (SEQ ID NO: 14) |
| | | 2 | SYNZIP22 | SYYHHHHHHLESTSLYKKAGS |
| | | | | GSNLLATLRSTAAVLENENHV |
| | | | | LEKEKEKLRKEKEQLLNKLEA |
| | | | | YK (SEQ ID NO: 13) |
| Beta-Ketothiolase | 6 | 1 | GYF | PATSQHPPPPPGHRSQAPSH |
| (bktB) | | | | (SEQ ID NO: 42) |
| | | 2 | PAH | ELNSLLILLEAAEYLERRDR |
| | | | | (SEQ ID NO: 43) |
| HMG-COA Synthase | 7 | 1 | WW1A | FQMPADTPPPAYLPPEDPMT |
| | | | | (SEQ ID NO: 44) |
| | | 2 | WW1B | ERESNEEPPPPYEDPYWGNG |
| | | | | (SEQ ID NO: 45) |
| HMG-COA Reductase | 8 | 1 | FOS | SYYHHHHHHLESTSLYKKAGS |
| | | | | GSQKVESLKQKIEELKQRKAQL |
| | | | | KNDIANLEKEIAYAET (SEQ ID |
| | | | | NO: 20) |
| | | 2 | SYNZIP9 | SYYHHHHHHLESTSLYKKAGS |
| | | | | EFFRRERNKMAAAKCRNRRRE |
| | | | | LTDTLQAETDQLEDEKSALQTE |
| | | | | IANLLKEKEKLEFILAAHRPAC |
| | | | | KIPDDLGFPEEMSLE (SEQ ID |
| | | | | NO: 19) |
| Mevalonate Kinase | 9 | 1 | VHS1 | VSSTKLVSFHDDSDEDLLHI |
| | | | | (SEQ ID NO: 46) |
| | | 2 | VHS2 | AAATPISTFHDDSDEDLLHV |
| | | | | (SEQ ID NO: 47) |
| Phosphomevalonate | 10 | 1 | SYNZIP13 | SYYHHHHHHLESTSLYKKAGS |
| Kinase | | | | GSFENVTHEFILATLENENAKL |
| | | | | RRLEAKLERELARLRNEVAWL |
| | | | | (SEQ ID NO: 24) |
| | | 2 | SYNZIP15 | SYYHHHHHHLESTSLYKKAGS |
| | | | | GSQKVEELKNKIAELENRNAV |
| | | | | KKNRVAHLKQEIAYLKDELAA |
| | | | | HEFE (SEQ ID NO: 23) |
| Diphosphomevalonate | 11 | 1 | MATH | HDDSLPHPQQATDDSGHESD |
| Decarboxylase | | | | (SEQ ID NO: 48) |
| | | 2 | SKP1 | GSPNAGSVEQTPKKPGLRRR |
| | | | | (SEQ ID NO: 49) |
| Isopentenyl- | 12 | 1 | SYNZIP5 | SYYHHHHHHLESTSLYKKAGS |
| Diphosphate Delta- | | | | GSQKVAQLKNRVAYKLKENA |
| Isomerase | | | | KLENIVARLENDNANLEKDIAN |
| | | | | LEKDIANLERDVAR (SEQ ID |
| | | | | NO: 28) |
| | | 2 | SYNZIP6 | SYYHHHHHHLESTSLYKKAGS |
| | | | | GSNTVKELKNYIQELEERNAEL |
| | | | | KNLKEHLKFAKAELEFELAAH |
| | | | | KFE (SEQ ID NO: 27) |
| Geranyl-Diphosphate | 13 | 1 | PDZ1 | TDEEREETEEEVYLLNSTTL |
| Synthase | | | | (SEQ ID NO: 50) |
| | | 2 | PDZ2 | DGNVSGTQRLDSATVRTYSC |
| | | | | (SEQ ID NO: 51) |
| Olivetol Synthase | 14 | 1 | SH2A | ALVDDAADYEPPPSNNEEAL |
| | | | | (SEQ ID NO: 52) |
| | | 2 | SH2B | RELFDDPSYVNVQNLDKARQ |
| | | | | (SEQ ID NO: 53) |
| Olivetolic Acid | 15 | 1 | PTB1 | KNTKSMNFDNPVYRKTTEEE |
| Cyclase | | | | (SEQ ID NO: 54) |
| | | 2 | PTB2 | RSLPSTWIENKLYGMSDPNW |
| | | | | (SEQ ID NO: 55) |
| CBGA Synthase | 16 | 1 | SH3A | VVDNSPPPALPPKKRQSAPS |
| | | | | (SEQ ID NO: 56) |
| | | 2 | SH3B | TORSKPQPAVPPRPSADLIL |
| | | | | (SEQ ID NO: 57) |
| Acetyl-CoA | 17 | 1 | FAT | SATRELDELMASLSDFKIQG |
| Carboxylase | | | | (SEQ ID NO: 58) |
| | | 2 | PEX | DLALSENWAQEFLAAGDAVD |
| | | | | (SEQ ID NO: 59) |

The spacers or linkers connecting an enzyme and ID, as well as a binding domain on a scaffold, can be peptide sequences ranging in length from 6 to 250 amino acid residues. The term “spacer” typically refers to a longer and more structurally-rigid peptide sequence and the term “linker” typically refers to a shorter and more structurally-flexible peptide sequence. In embodiments in which both terms are used, linker typically refers to a sequence that is about 3 to about 50 amino acids in length and spacer typically refers to a sequence that is longer (e.g., about 36 to about 250 amino acids in length). For example, a linker can be 6-15, 10-20, 15-25, 20-30, 25-35, 30-40, 35-45, or 40-50 amino acids in length. A spacer can be, for example, 36-40, 40-50, 45-55, 50-60, 55-65, 60-70, 65-75, 70-80, 75-85, 90-100, 95-105, 100-110, 105-115, 110-120, 115-125, 120-130, 125-135, 130-140, 135-145, 140-150, 145-155, 150-160, 165-175, 170-180, 175-185, 180-190, 185-195, 190-200, 195-205, 200-210, 205-215, 210-220, 215-225, 220-230, 225-235, 230-240, 235-245, or 240-250 amino acids in length. See, for example, Chen, et al., Adv Drug Deliv Rev. 2013 65(10): 1357-1369. In either case, the linker/spacer can be a series of small and/or hydrophilic and/or other amino acid residues that can adapt flexible and/or rigid structures. For example, the linker can be a series of glycine residues, a series of alanine residues, a series of serine residues, or a series of alternating glycine and serine (or threonine) residues such as (G-S)₈ (SEQ ID NO:60), (G-S)₁₀ (SEQ ID NO:61), or (G-S)₁₅ (SEQ ID NO:62), or contain mainly glycine residues such as (GGGGS)₃ (SEQ ID NO:63) or (GGGGS)₄ (SEQ ID NO:64), or contain any other series of canonical or non-canonical amino acid residues or combinations thereof. In some embodiments, a linker can include glutamic acid, alanine, and lysine residues such as (EAAAK)₂ (SEQ ID NO:65), (EAAAK)₃ (SEQ ID NO:66), or (EAAAK)₄ (SEQ ID NO:67). See, Horn and Sticht, 2015, supra. In some embodiments, a linker can be a combination of glycine, alanine, proline and methionine residues, such as AAAGGM (SEQ ID NO:68), AAAGGMPPAAAGGM (SEQ ID NO:69), AAAGGM (SEQ ID NO:70), or PPAAAGGMM (SEQ ID NO:71). See, e.g., U.S. Pat. No. 9,856,460.

Based on amino acid composition, linkers or spacers can be either structured or intrinsically unstructured. For example, in some embodiments, a spacer can have a sequence that adopts a more structurally-rigid α-helical conformation and a linker can have a GS-rich peptide sequence that is more structurally-flexible. For example, in some embodiments, a linker can include flexible GS-rich sequences flanking one or more rigid α-helical moieties, e.g., GS-rich sequences flanking duplicate, triplicate, or quadruplicate α-helical moieties. For example, in some embodiments, a linker or spacer can have the sequence GSAGSAAGSGEF (SEQ ID NO:72), KLSGGGGSGGGGSGGGGS (SEQ ID NO:73), GSAGSAAGSGEFGSAEAAAKEAAAKAGSAGSAAGSGEFGS (SEQ ID NO:74), GSAGSAAGSGEFAEAAAKEAAAKAGSAGSAAGSGEF (SEQ ID NO:75), or GSAGSAAGSGEFGSAEAAAKEAAAKEAAAKEAAAKAGSAGSAAGSGEFGS (SEQ ID NO:76).

In some embodiments, the ligands on the scaffold can be separated by linkers that are 20-50 amino acid residues in length (e.g., 20, 21, 22, 23, 24, 25, 26, 27, 28, 29, 30, 31, 32, 33. 34, 35, 36, 37, 38, 39, 40, 41, 42, 43, 44, 45, 46, 47, 48, 49, or 50 amino acid residues in length). In some embodiments, the IDs engineered at the C-terminus or N-terminus of each scaffolded enzyme can contain a linker (e.g., a flexible linker) of 15 to 30 (e.g., 20) amino acid residues in length flanking a spacer of 15 to 50 (e.g. 36) amino acid residues. In some embodiments, the ID can be separated from the enzyme by a spacer sequence such as the cTPR6 spacer, which includes sextuplicate rigid α-helical moieties and can have the sequence:

(SEQ ID NO: 77)
AEAWYNLGNAYYKQGDYQKAIEYYQKALELDPNNAEAWYNLGNAYYKQGD
YQKAIEYYQKALELDPNNAEAWYNLGNAYYKQGDYQKAIEDYQKALELDP
NNLQAEAWKNLGNAYYKQGDYQKAIEYYQKALELDPNNASAWYNLGNAYY
KQGDYQKAIEYYQKALELDPNNAKAWYRRGNAYYKQGDYQKAIEDYQKAL
ELDPNNRSRSA.

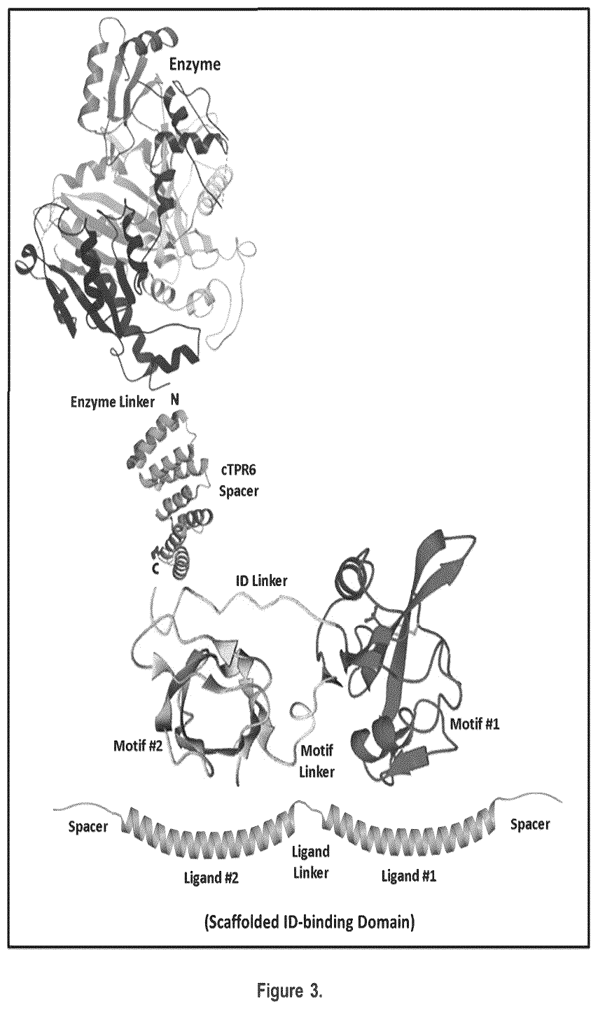

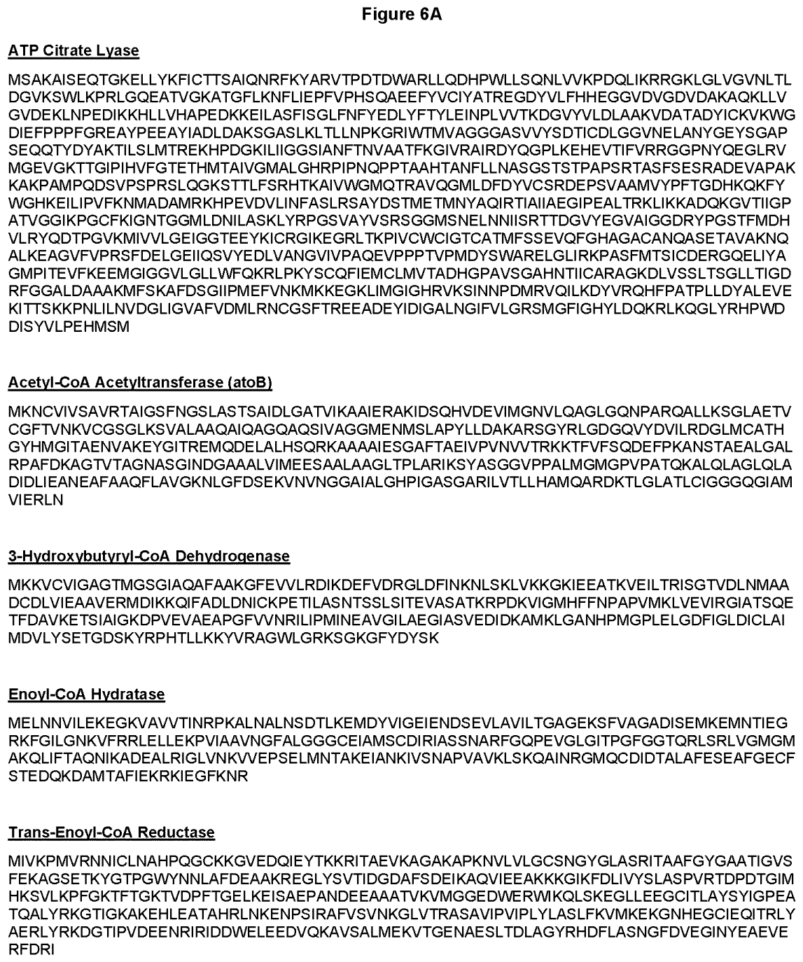

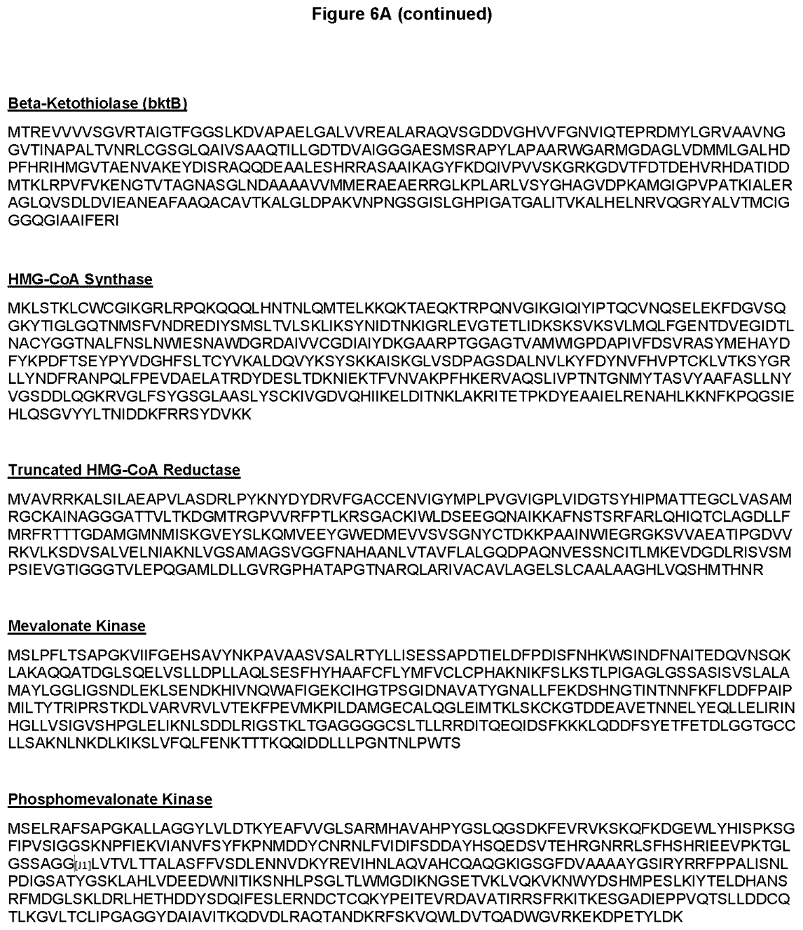

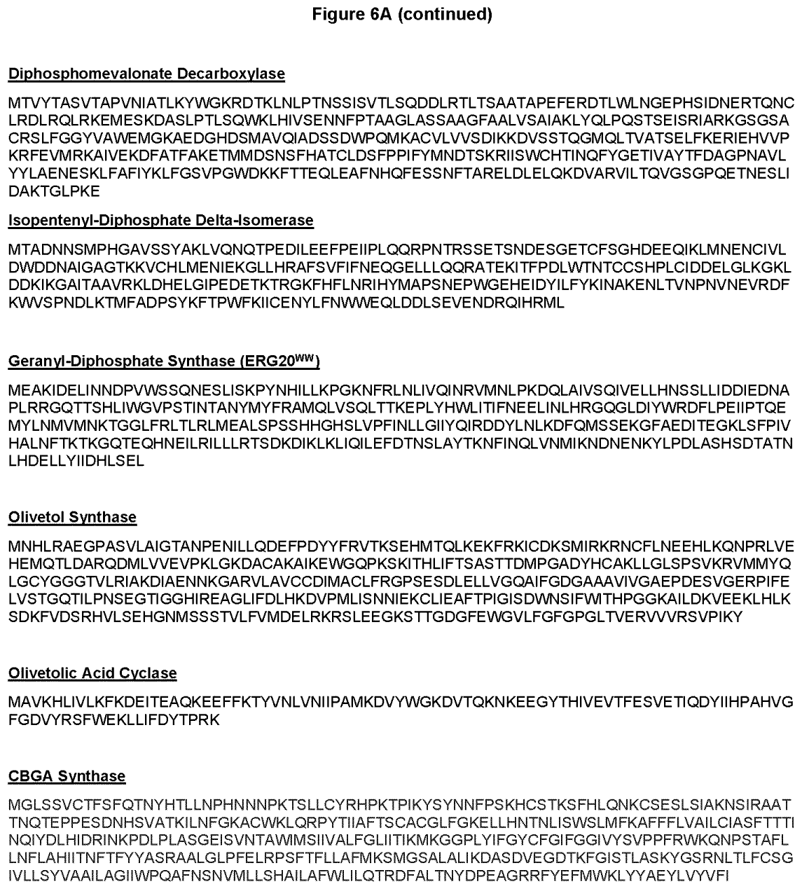

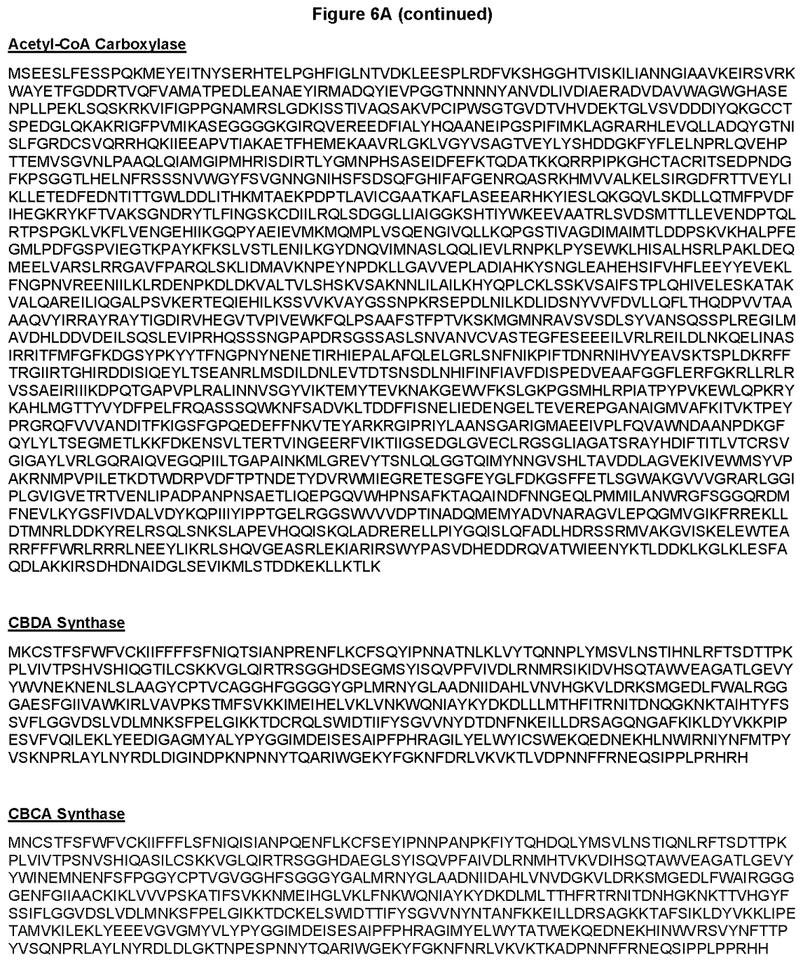

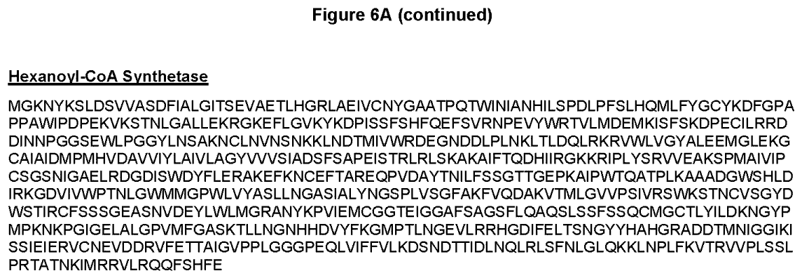

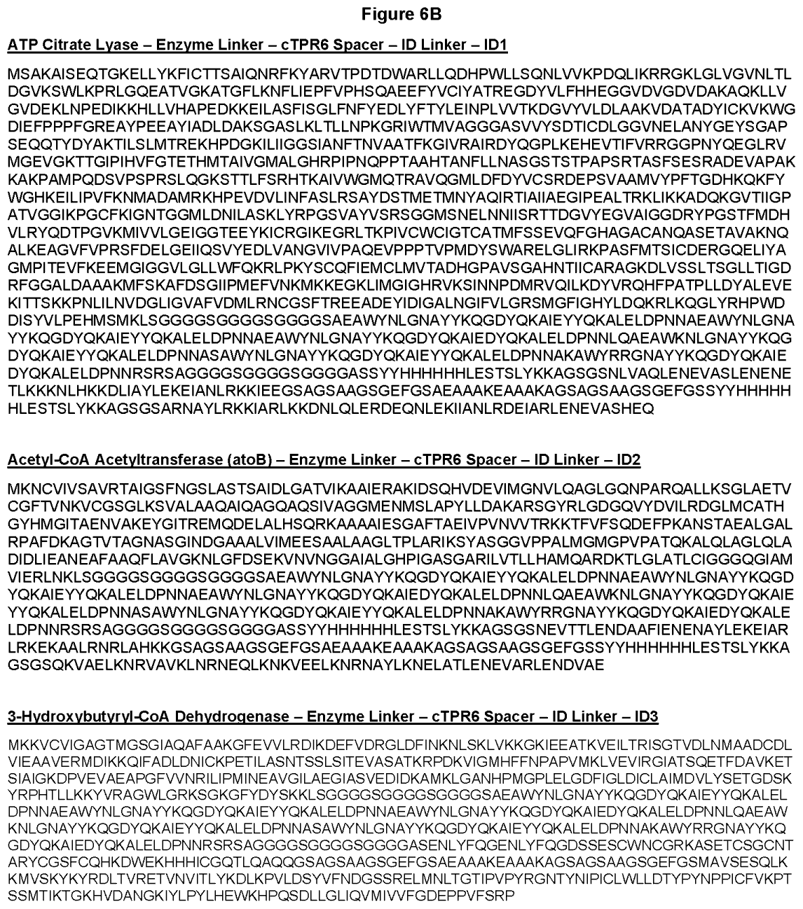

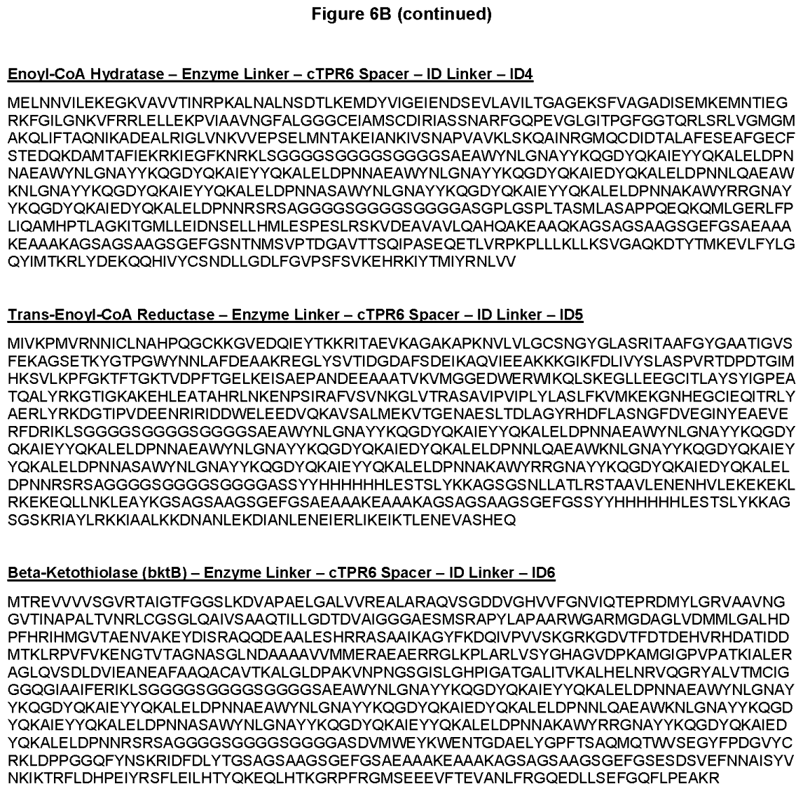

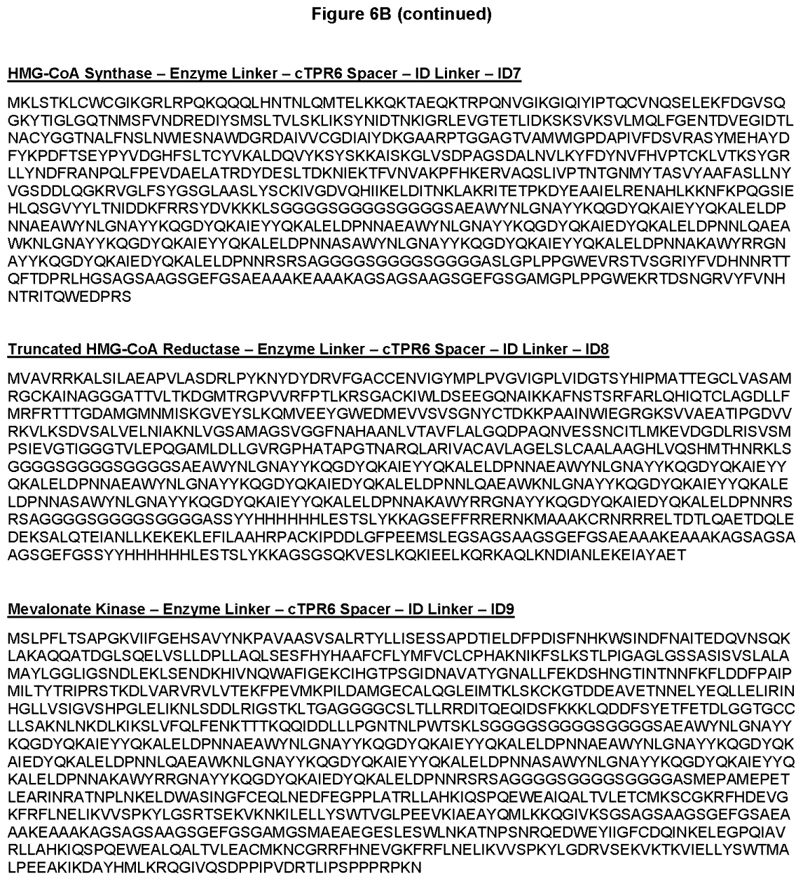

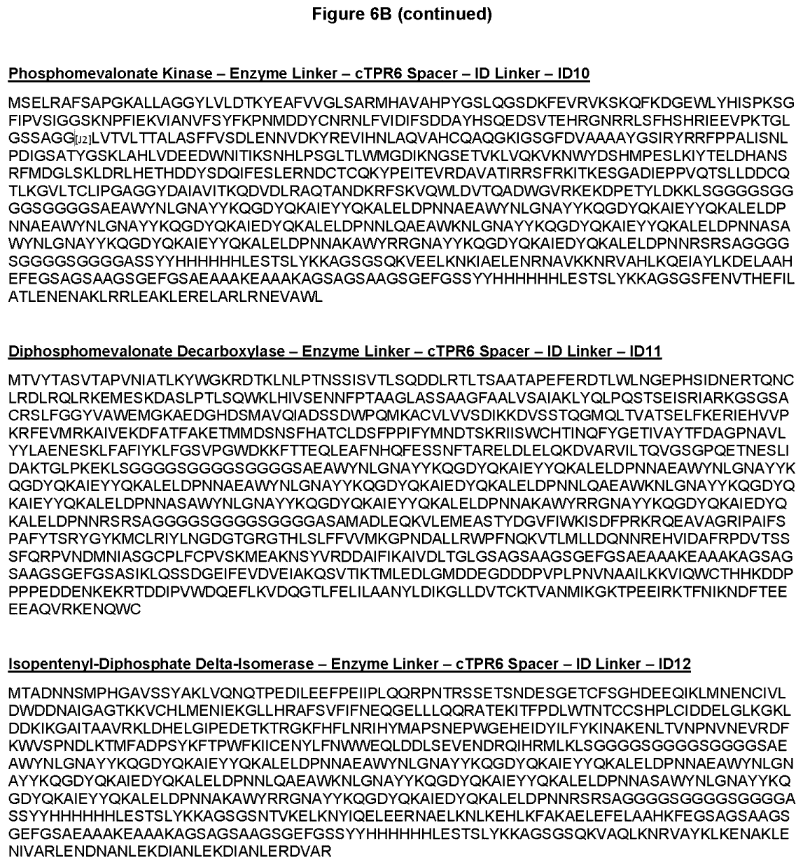

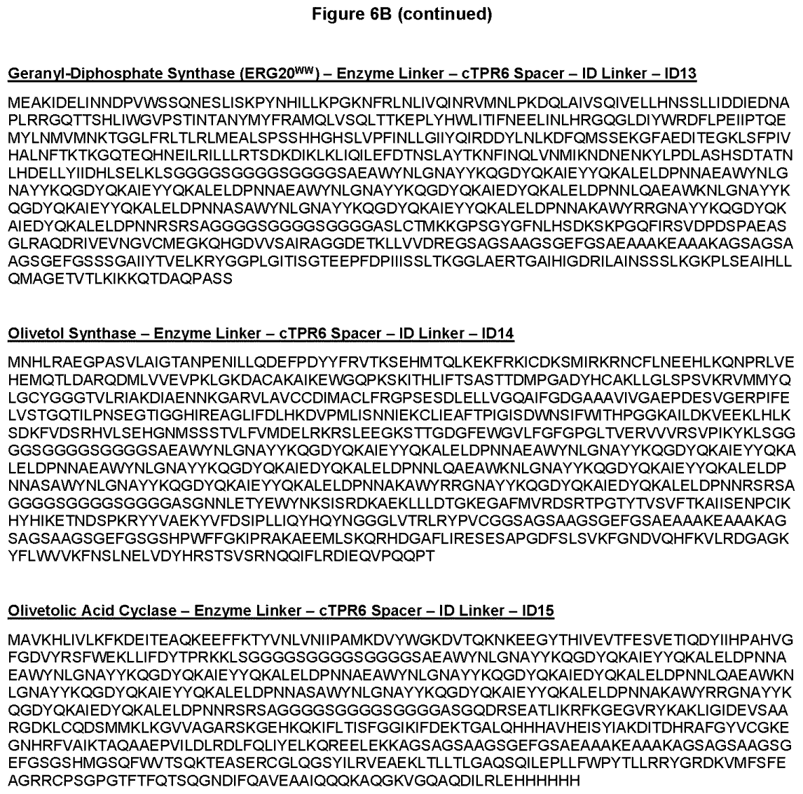

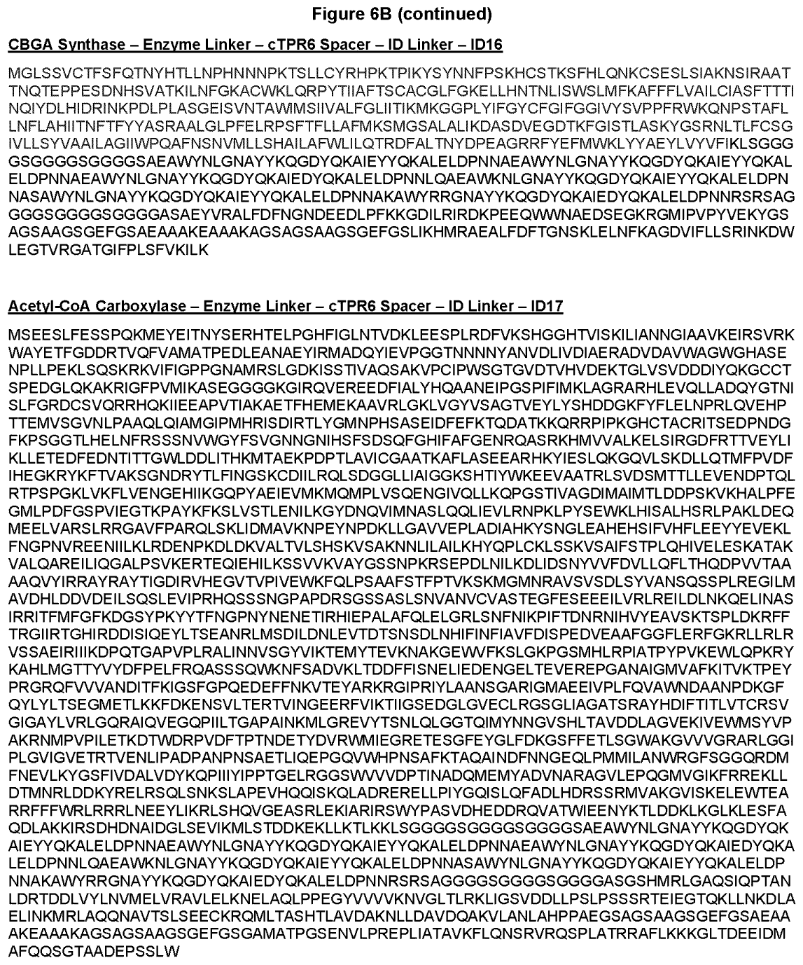

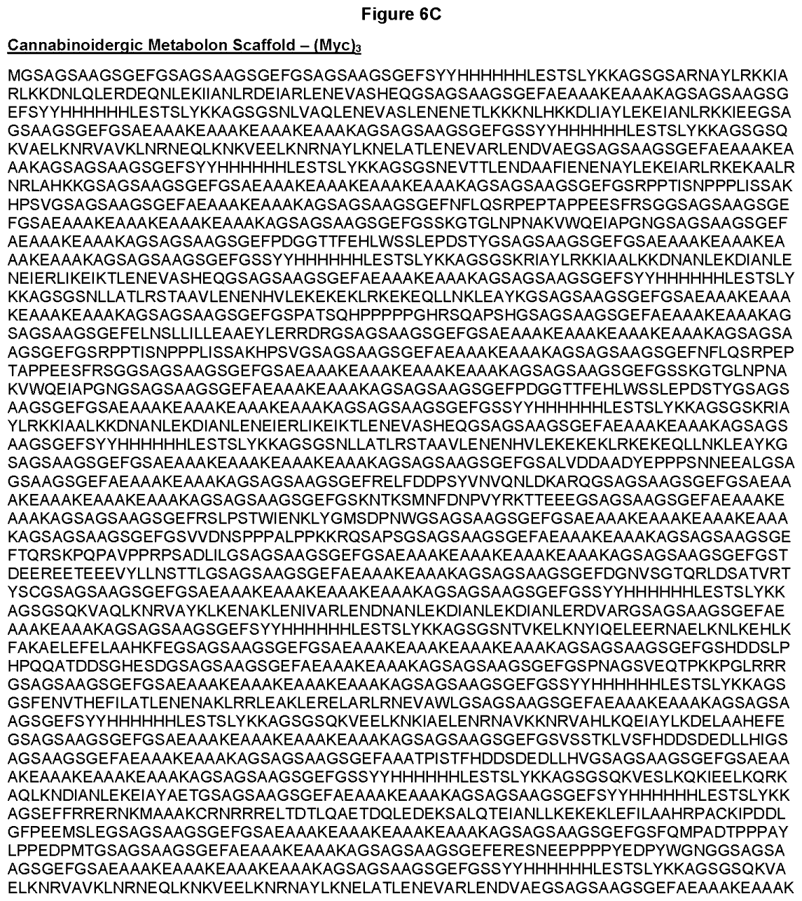

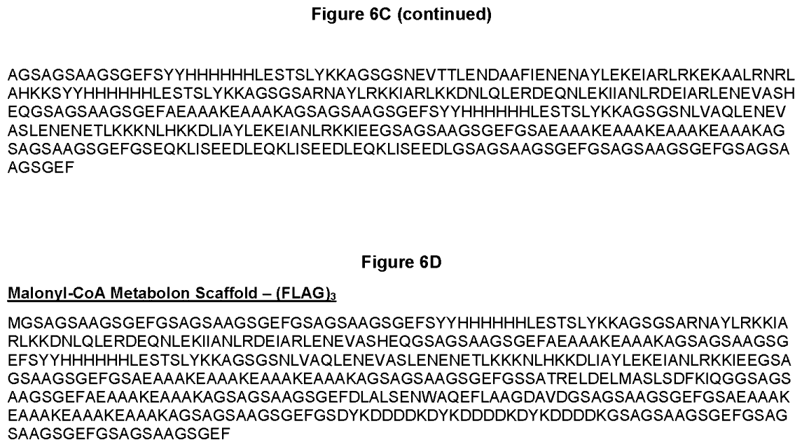

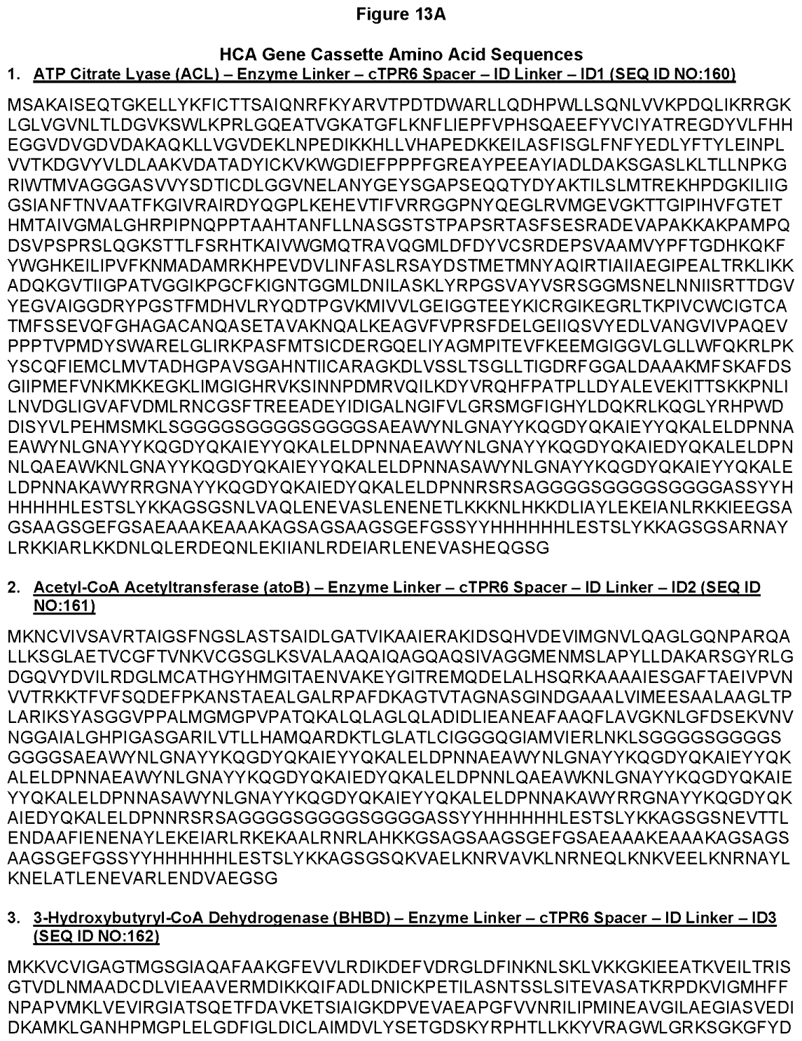

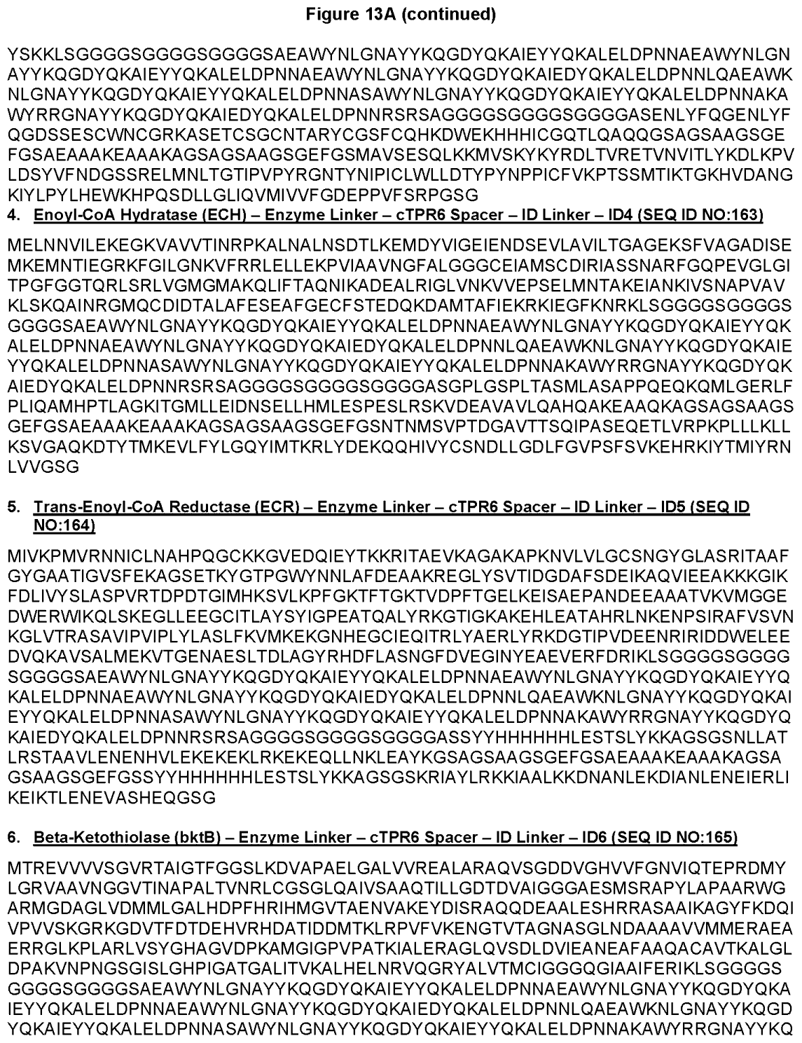

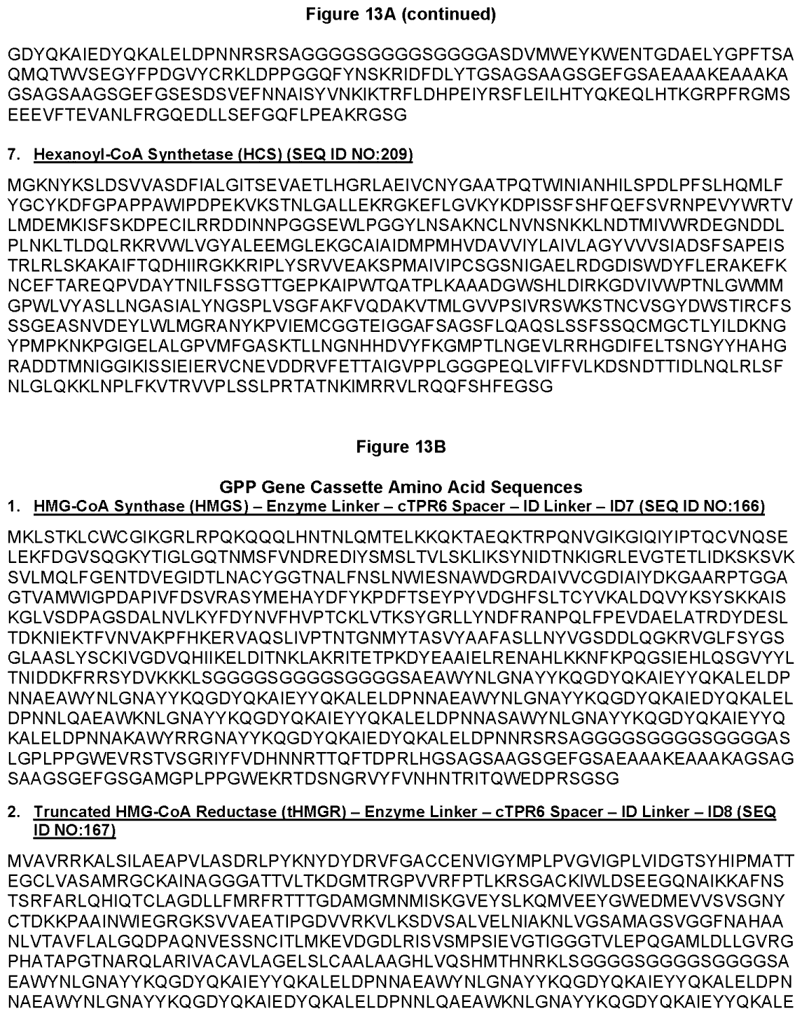

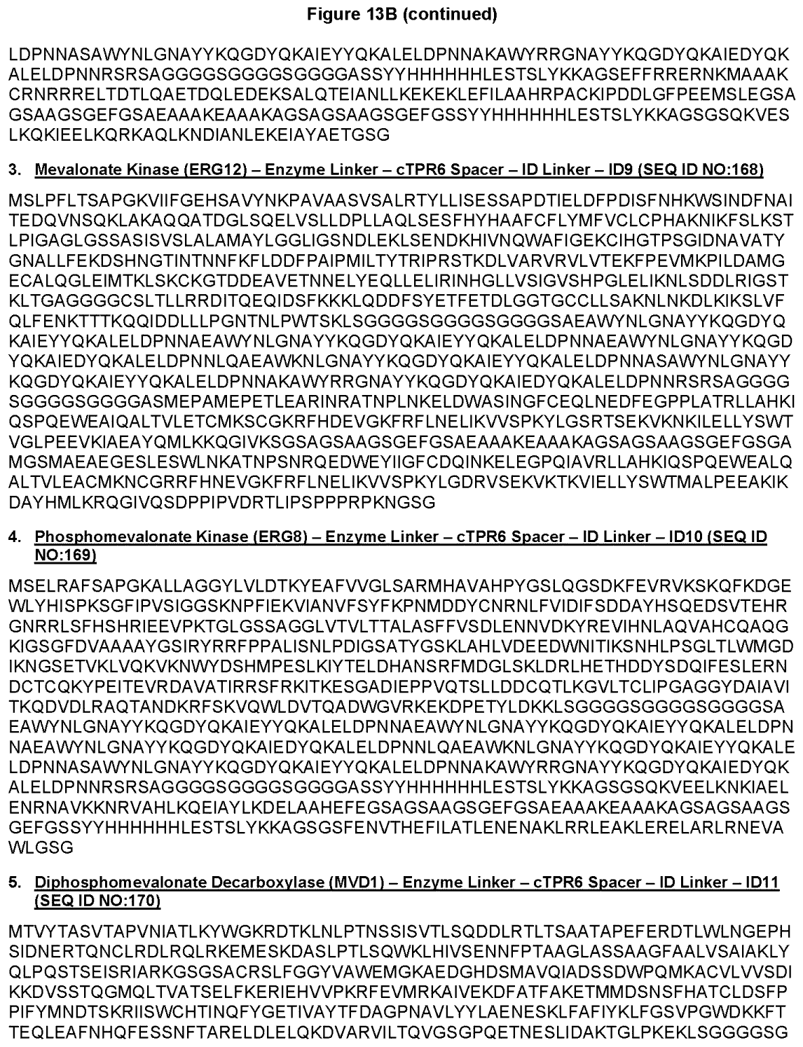

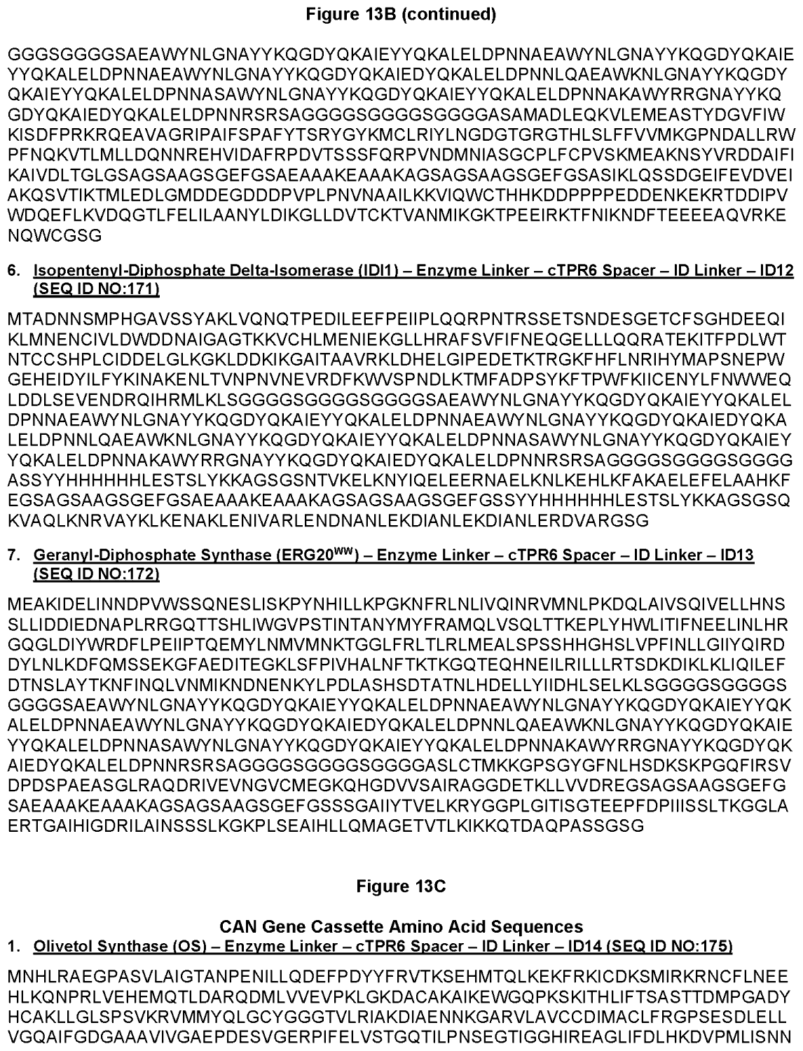

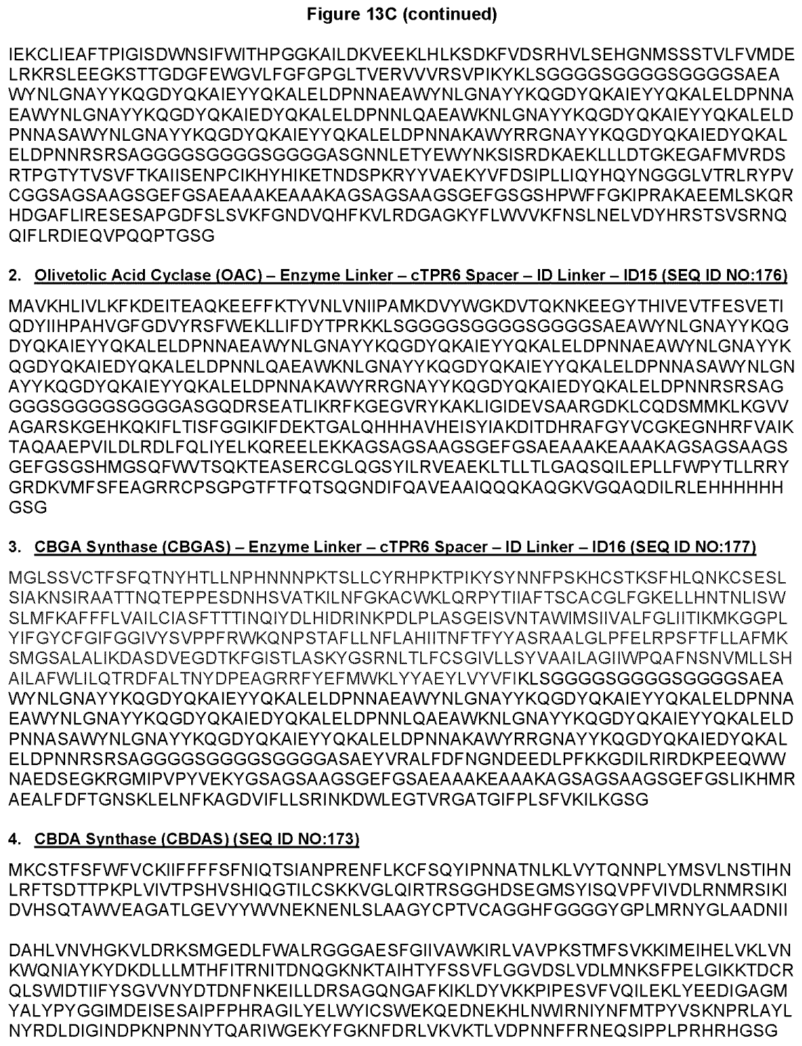

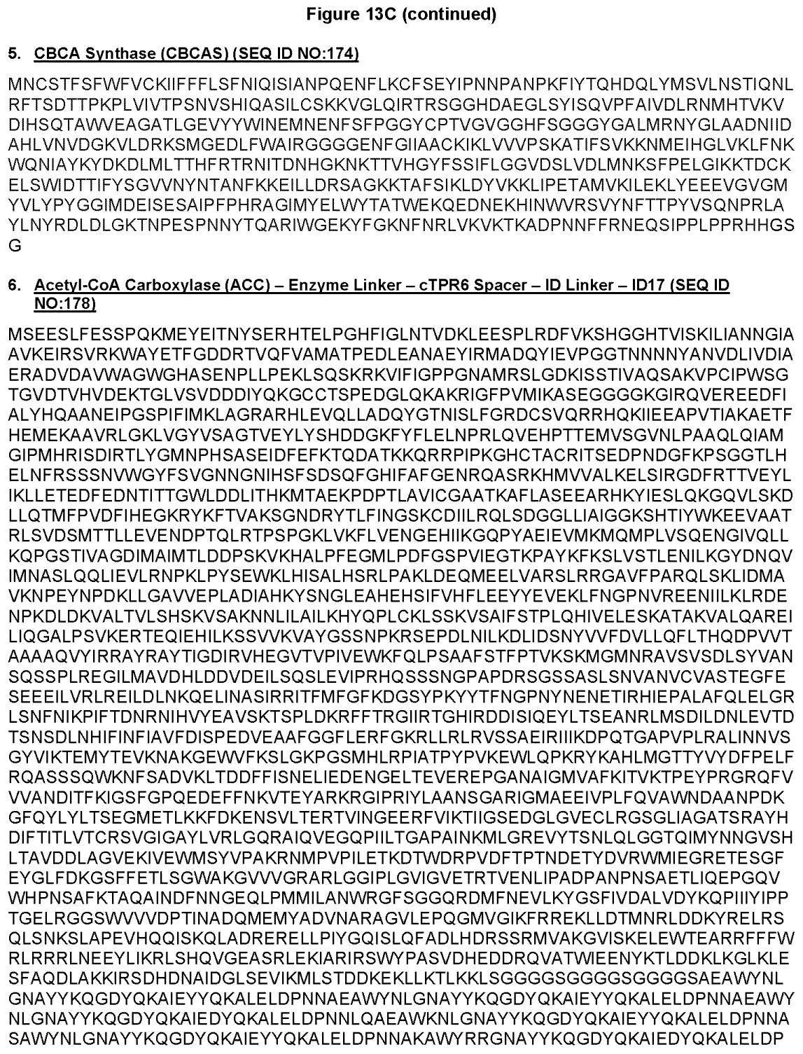

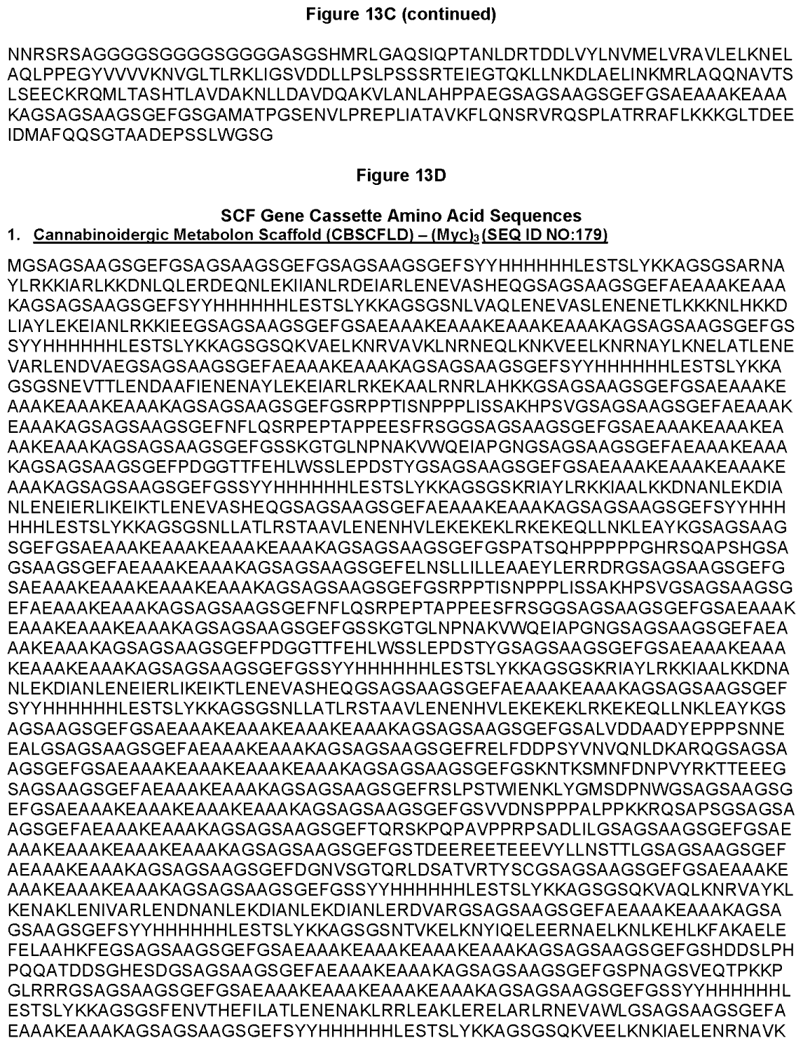

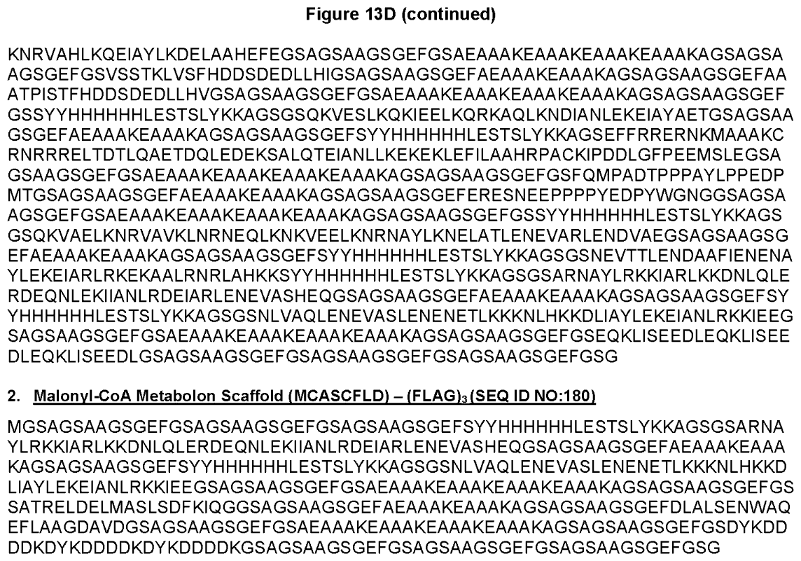

In some embodiments, the engineered enzyme can be of a formula: enzyme—linker₁—spacer—linker₂—motif₁—linker₃—motif₂, where linkers 1, 2, and 3 can be the same or different, and motif 1 and motif 2 can be the same or different. In some embodiments, linker 1 can be referred to as the enzyme linker, i.e., it connects the enzyme to the spacer such as cTPR6 spacer, and can include flexible GS-rich moieties flanking a rigid α-helical moiety such as KLSGGGGSGGGGSGGGGS (SEQ ID NO:73). In some embodiments, linker 2 can be referred to as the ID linker and can include, for example, flexible GS-rich moieties flanking a rigid α-helical moiety such as GGGGSGGGGSGGGGAS (SEQ ID NO:78). In some embodiments, linker 3 can be referred to as the motif linker and can include flexible GS-rich moieties flanking a rigid α-helical moiety such as GSAGSAAGSGEFGSAEAAAKEAAAKAGSAGSAAGSGEFGS (SEQ ID NO:74). Table 1 provides non-limiting examples of motifs 1 and motifs 2, which are used together to form heterologous IDs. FIG. 3 contains a schematic of an exemplary engineered enzyme of this formula complexed with a scaffold. FIG. 6B and FIGS. 13A-C contain the amino acid sequence of an ATP citrate lyase, atoB, a 3-hydroxybutyryl-CoA dehydrogenase, an enoyl-CoA hydratase, a trans-enoyl-CoA reductase, a beto-ketothiolase (bktB), an HMG-CoA synthase, a truncated HMG-CoA reductase, a mevalonate kinase, a phosphomevalonate kinase, a diphosphomevalonate decarboxylase, an isopentenyl-diphosphate delta isomerase, a geranyl-diphosphate synthase (ERG20^{WW}), an olivetol synthase, an olivetolic acid cyclase, a CBGA synthase, and an acetyl-CoA carboxylase according to this formula. In some embodiments, linkers 1 and 2 can be (G₄S)₃, the spacer can be the cTPR6 sequence, and linker 3 can be (GS)₈.

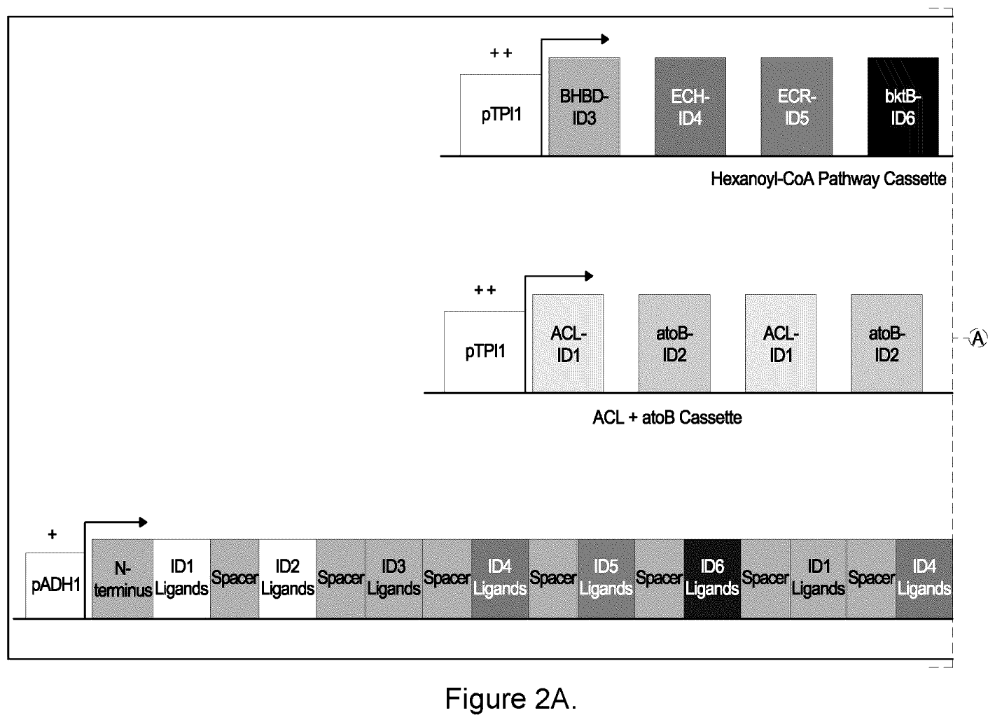

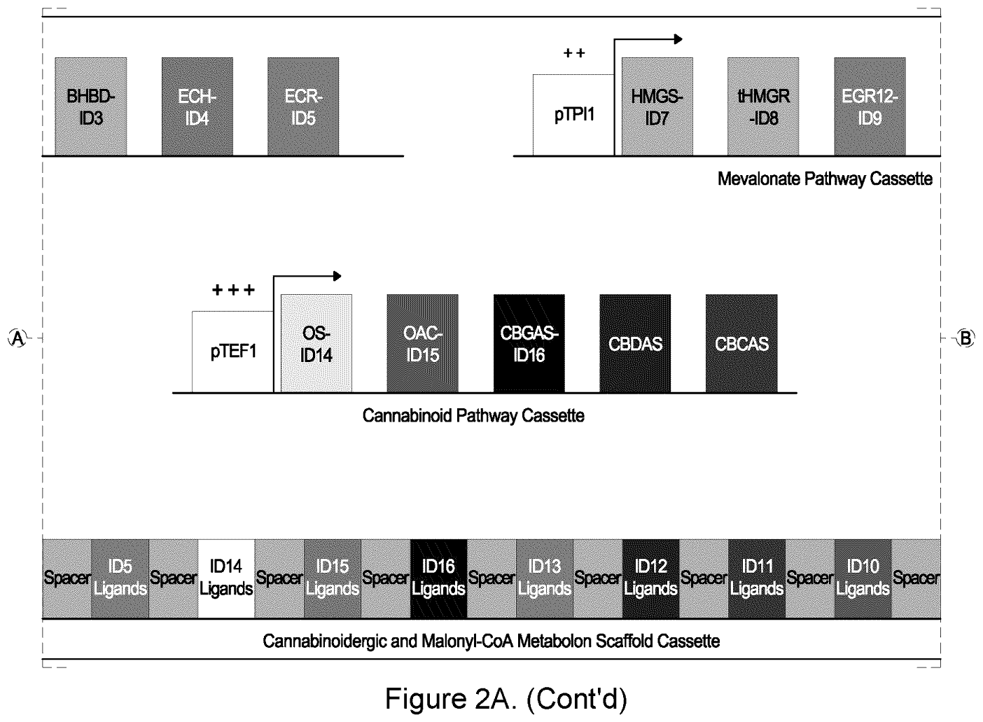

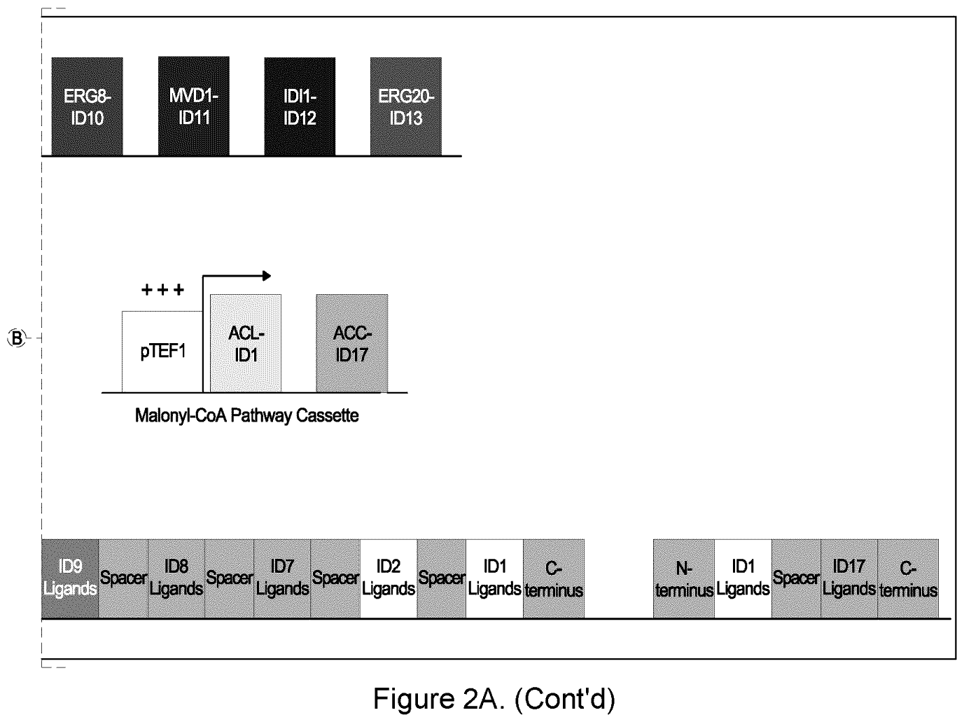

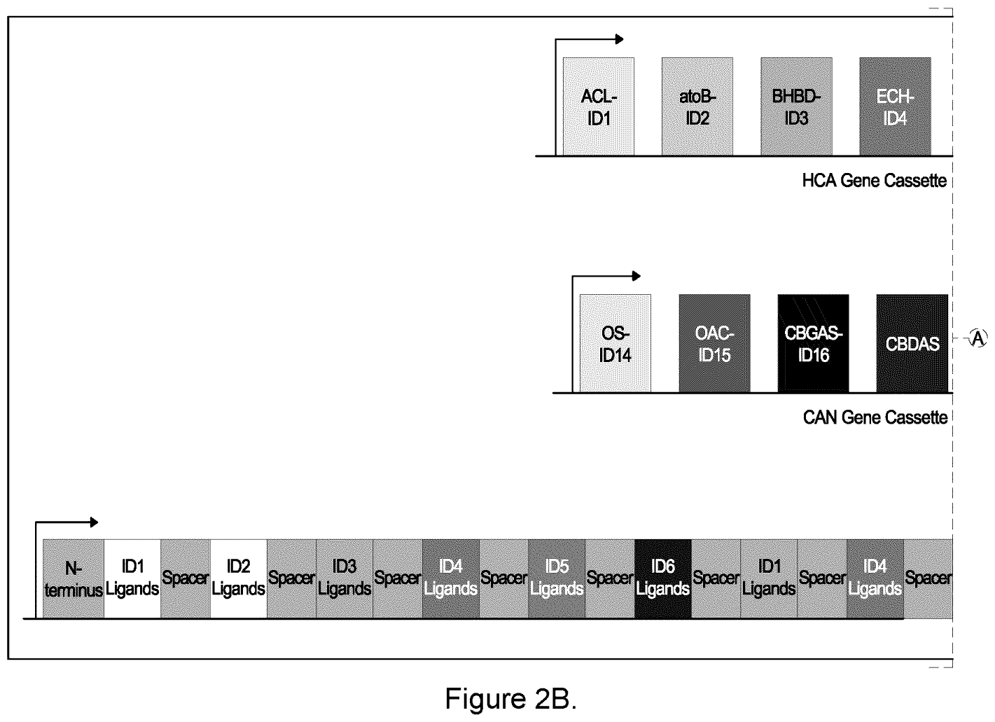

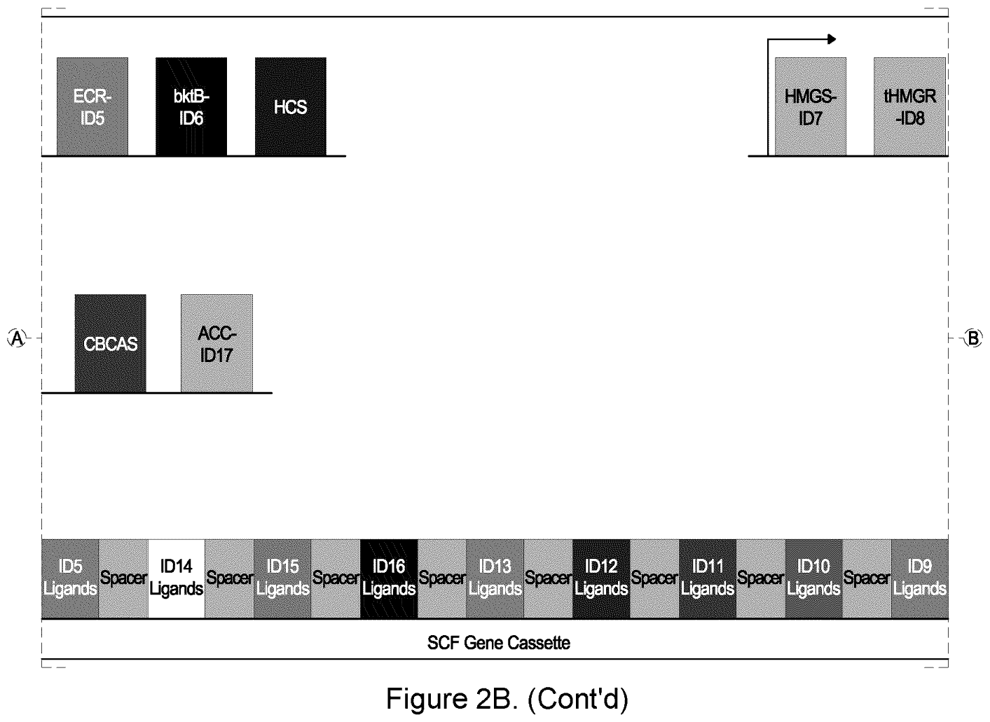

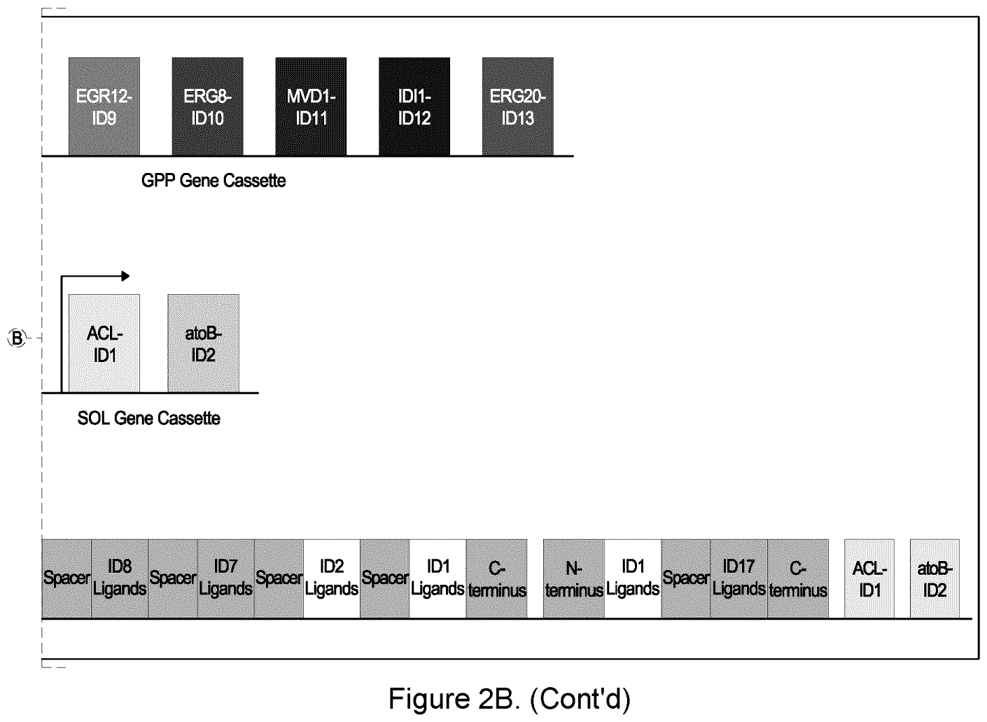

In some embodiments, a scaffold can be of a formula: N-terminus—[Ligand #1—linker—Ligand #2—Spacer]n—(optionally-tagged) C-terminus, where n is the number of interaction domains. The linker can be referred to as a scaffolded ligand linker and can be used to connect and separate paired motif-binding ligands that recruit/localize each enzyme to its scaffold-binding site. Such a linker can include flexible GS-rich moieties flanking a rigid α-helical moiety and have a sequence such as GSAGSAAGSGEFAEAAAKEAAAKAGSAGSAAGSGEF (SEQ ID NO:75). The spacer can be referred to as a scaffolded ID-binding site spacer and can be used to connect and separate the scaffold-binding sites (composed of the paired motif binding ligands) for each enzyme. Such a spacer can include flexible GS-rich moieties flanking a rigid α-helical moiety and have a sequence such as GSAGSAAGSGEFGSAEAAAKEAAAKEAAAKEAAAKAGSAGSAAGSGEFGS (SEQ ID NO:76). The N-terminus can include a flexible GS-rich sequence to help stabilize and solubilize the scaffold. For example, the N-terminus can have the sequence GSAGSAAGSGEFGSAGSAAGSGEFGSAGSAAGSGEF (SEQ ID NO:79). The C-terminus can include a flexible GS rich sequence flanking a rigid α-helical moiety to stabilize and solubilize the scaffold and can be optionally tagged (e.g., with a MYC tag, a FLAG tag, or other tag described below) to ease purification or detection of the scaffold. For example, a C-terminal sequence with a triplicate MYC tag can have the sequence GSAGSAAGSGEFGSAEAAAKEAAAKEAAAKEAAAKAGSAGSAAGSGEFGSEQK LISEEDLEQKLISEEDLEQKLISEEDLGSAGSAAGSGEFGSAGSAAGSGEFGSAGS AAGSGEF (SEQ ID NO:80). For example, a C-terminal sequence with a triplicate FLAG tag can have the sequence GSAGSAAGSGEFGSAEAAAKEAAAKEAAAKEAAAKAGSAGSAAGSGEFGSDYK DDDDKDYKDDDDKDYKDDDDKGSAGSAAGSGEFGSAGSAAGSGEFGSAGSAA GSGEF (SEQ ID NO:81). FIG. 6C and FIG. 13D each contain an example of a scaffold polypeptide of this formula that contains the peptide ligands corresponding to IDS 1-16 as shown in Table 2, and a triplicate MYC tag on the C-terminus. For example, FIG. 13D contains an example of a scaffold polypeptide (see SCF gene cassette of FIG. 2B) containing a triplicate MYC tag. FIG. 6D and FIG. 13D each contain an example of a scaffold polypeptide that contains the peptide ligands corresponding to IDs 1 and 17 as shown in Table 2 and a triplicate FLAG tag on the C-terminus. Accordingly, the amino acid sequence of a scaffold can depend on the sequence of the peptide ligands that can bind to the selected ID motif of the enzymes.

In some embodiments, any one of the enzymes can be engineered to include an N-terminal or C-terminal linker motif that allows covalent (isopeptide) bonding to the scaffold. See, for example, the SpyTag and SpyCatcher system described by Zakeri, et al., Proc. Natl. Acad. Sci., 2012 109(12) E690-E697.

In some embodiments involving multi-enzymatic scaffolds described herein, the first engineered enzyme of a biosynthetic pathway can produce a first product that can be a substrate for the second engineered enzyme of the biosynthetic pathway, the second engineered enzyme of the biosynthetic pathway can produce a second product that can be a substrate for the third engineered enzyme of the biosynthetic pathway, and so forth. In some cases, the second engineered enzyme can be immobilized on the scaffold such that it is positioned adjacent to or very close to the first engineered enzyme. The third engineered enzyme can be immobilized on the scaffold such that it is positioned adjacent or very close the second engineered enzyme. In this way, the effective concentration of the first product can be high, and the second engineered enzyme can act efficiently on the first product, the third engineered enzyme can act efficiently on the second product, and so forth.

As shown in FIGS. 1A and 1B, one example of a multi-enzymatic scaffold contains enzymes of the hexanoyl-CoA pathway on the N-terminus of the scaffold, enzymes of the mevalonate pathway on the C-terminus of the scaffold, and enzymes of the upper cannabinoid pathway in between. Within any of the pathways, the enzymes can be from a single source, i.e., from one species or genera, or can be from multiple sources, i.e., different species or genera. Nucleic acids encoding the enzymes described herein have been identified from various organisms and are readily available in publicly available databases such as GenBank or EMBL (see below).

A fully-assembled multi-enzymatic scaffold provided herein can adopt stoichiometry and a spatial arrangement that can help maximize pathway flux and minimize accumulation of pathway intermediates and by-products. Such scaffolds can facilitate substrate channeling both within and between cannabinoid and cannabinoid precursor pathways. Specifically, this scaffolding system can facilitate unidirectional flux through each of the primary cannabinoid precursor pathways, and converging near the midpoint of the scaffold. The hexanoyl-CoA/olivetolic acid (OVA) pathway can begin at the N-terminus of the scaffold, and the mevalonate or MEP pathway can begin at the C-terminus of the scaffold. The enzyme catalyzing the rate-limiting/committed step in cannabinoid biosynthesis, a CBGA synthase, can be localized at the intersection of these precursor pathways near the scaffold midpoint.

By this design, the two primary precursors for cannabinoid biosynthesis, hexanoyl-CoA/olivetolic acid and geranyl pyrophosphate, can be bi-directionally delivered to a CBGA synthase at this intersection. The CBGA synthase can catalyze biosynthesis of CBGA, the primary cannabinoid from which all other cannabinoids are derived. Substrate channeling within and between the scaffolded pathways can accelerate the kinetics of the composite pathway in accordance with the law of mass action.

In the embodiment shown in FIGS. 1A and 1B, the N-terminal hexanoyl-CoA pathway can include an ATP citrate lyase (ACL) (also can be referred to as an ATP citrate synthase), an acetyl-CoA acetyltransferase (atoB), two 3-hydroxy-acyl-CoA dehydrogenases (BHBDs), two enoyl-CoA hydratases (ECHs), a beta-ketothiolase (bktB), and two trans-2-enoyl-CoA-reductases (ECRs).

In the hexanoyl-CoA pathway shown in FIGS. 1A and 1B, citrate, from cellular metabolism and/or supplemented in the growth medium, can be used as a substrate for ACL-catalyzed acetyl-CoA synthesis. ACL is classified under EC 2.3.3.8. Acetyl-CoA can be used as a substrate for atoB-catalyzed acetoacetyl-CoA synthesis. atoB is classified under EC 2.3.1.9. Acetoacetyl-CoA can serve as the substrate for BHBD-catalyzed 3-hydroxybutanoyl-CoA synthesis. BHBD is classified under EC 1.1.1.157. 3-hydroxybutanoyl-CoA can serve as the substrate for ECH-catalyzed trans-but-2-enoyl-CoA synthesis. ECH is classified under EC 4.2.1.17. Trans-but-2-enoyl-CoA can serve as the substrate for ECR-catalyzed butanoyl-CoA synthesis. ECR is classified under EC 1.3.8.1. Butanoyl-CoA can serve as the substrate for bktB-catalyzed 3-keto-hexanoyl-CoA synthesis. bktB is classified under EC 2.3.1.9. The bktB catalyzing the production of 3-ketohexanoyl CoA from butanoyl-CoA can be the same as, or different from, the atoB used to catalyze the production of acetoacetyl-CoA from acetyl-CoA. 3-ketohexanoyl-CoA is the substrate for BHBD-catalyzed 3-hydroxyhexanoyl-CoA synthesis. BHBD is classified under EC 1.1.1.157. The BHBD catalyzing the production of 3-hydroxyhexanoyl-CoA can be the same as, or different from, the BHBD used to catalyze the production of 3-hydroxybutanoyl-CoA. 3-hydroxyhexanoyl-CoA can be the substrate for ECH-catalyzed trans-hex-2-enoyl-CoA synthesis. ECH is classified under 4.2.1.17. The ECH catalyzing the production of trans-hex-2-enoyl-CoA can be the same as, or different from, the ECH used to catalyze the production of trans-but-2-enoyl-CoA. Trans-hex-2-enoyl-CoA can be the substrate for ECR-catalyzed hexanoyl-CoA synthesis. ECR is classified under EC 1.3.1.38 or EC 1.3.1.44. The ECR catalyzing the production of hexanoyl-CoA can be the same as, or different from, the ECR used to catalyze the production of butanoyl-CoA

In some embodiments, a hexanoyl-CoA synthetase (HCS) enzyme can be substituted for the scaffolded enzymes of the hexanoyl-CoA pathway or can be included in a soluble form in addition to the scaffolded enzymes of the hexanoyl-CoA pathway, and in some embodiments, hexanoic acid can be added to the growth media as a substrate for HCS-catalyzed hexanoyl-CoA production. The HCS can be included on the scaffold, N-terminal to the upper cannabinoid pathway in FIGS. 1A and 1B, and/or it can be non-scaffolded (soluble).

In the embodiment shown in FIGS. 1A and 1B, the C-terminal mevalonate pathway can include an ACL, an atoB, a hydroxymethylglutaryl-CoA, an HMG-CoA synthase (HMGS), an HMG-CoA reductase (HMGR), a mevalonate kinase (ERG12), a phosphomevalonate kinase (ERG8), a diphospho mevalonate decarboxylase (MVD1), an isopentyl diphosphate isomerase (IDI1), and a mutant GPP synthase (mGPPS). In the mevalonate pathway shown in FIGS. 1A and 1B, citrate from cellular metabolism and/or supplemented in the growth medium, can be used as a substrate for ACL-catalyzed acetyl-CoA synthesis. ACL is classified under EC 2.3.3. Acetyl-CoA can be used as a substrate for bktB-catalyzed acetoacetyl-CoA synthesis. bktB is classified under EC 2.3.1.9. Acetoacetyl-CoA can be the substrate for HMGS-catalyzed HMG-CoA synthesis. HMG-CoA can be the substrate for HMGR catalyzed mevalonate synthesis. HMGR is classified under EC 1.1.1.88 or 1.1.1.34. Mevalonate can be the substrate for mevalonate kinase-catalyzed mevalonate-5 phosphate synthesis. Mevalonate kinase is classified under EC 2.7.1.36. Mevalonate-5-phosphate can be the substrate for phosphomevalonate kinase-catalyzed mevalonate pyrophosphate synthesis. Phosphomevalonate kinase is classified under EC 2.7.4.2. Mevalonate pyrophosphate can be the substrate for diphosphomevalonate decarboxylase-catalyzed isopentyl pyrophosphate synthesis. Diphosphomevalonate decarboxylase is classified under EC 4.1.1.33. Isopentyl pyrophosphate can be the substrate for isopentyl diphosphate isomerase-catalyzed dimethylallyl pyrophosphate synthesis. Isopentyl diphosphate isomerase is classified under EC. 5.3.3.2. Dimethylallyl pyrophosphate can be the substrate for geranyl pyrophosphate synthase (GPPS)-catalyzed geranyl pyrophosphate synthesis. GPPS is classified under EC 2.5.1.1.

As acetyl-CoA can be the initial substrate for the hexanoyl-CoA, mevalonate/geranyl pyrophosphate, and malonyl-CoA cannabinoid precursor biosynthetic pathways, the inclusion of ACL at both the N-terminus and C-terminus of the multi-enzymatic scaffold in FIGS. 1A and 1B can directly couple the scaffolded pathways to cellular metabolism via ACL-catalyzed production of acetyl-CoA from citric acid cycle-derived citrate. The citrate also can be supplemented into the culture medium (e.g., as buffered citrate). In some embodiments, the ACL enzyme is included only at the N-terminus of the scaffold. In some embodiments, the ACL enzyme is included only at the C-terminus of the scaffold. In some embodiments, the ACL enzyme is included in soluble form.

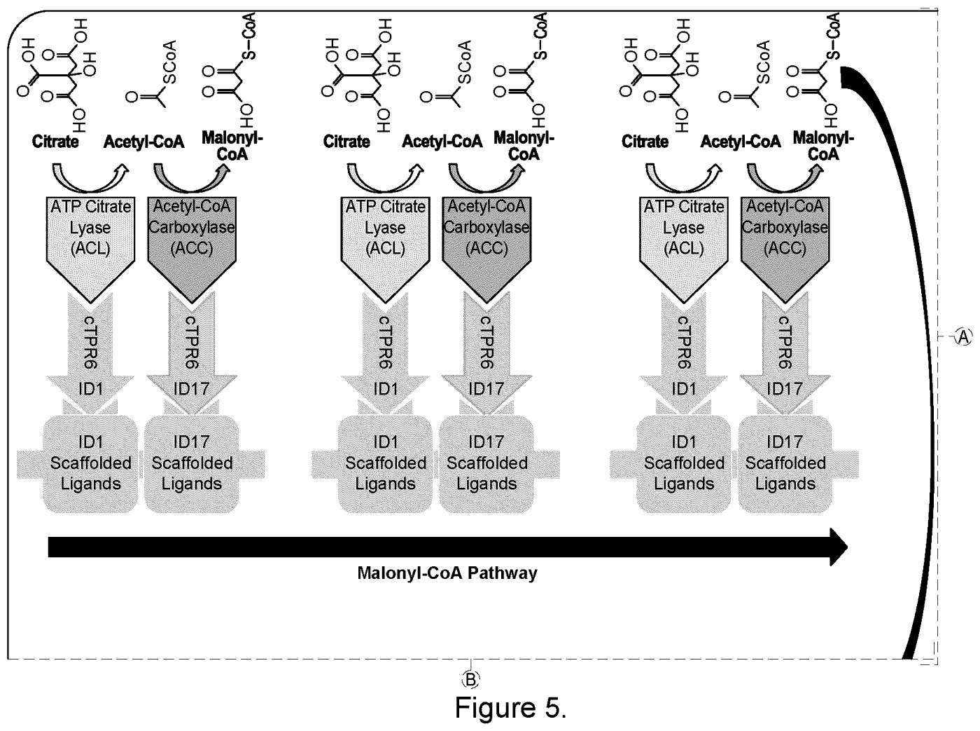

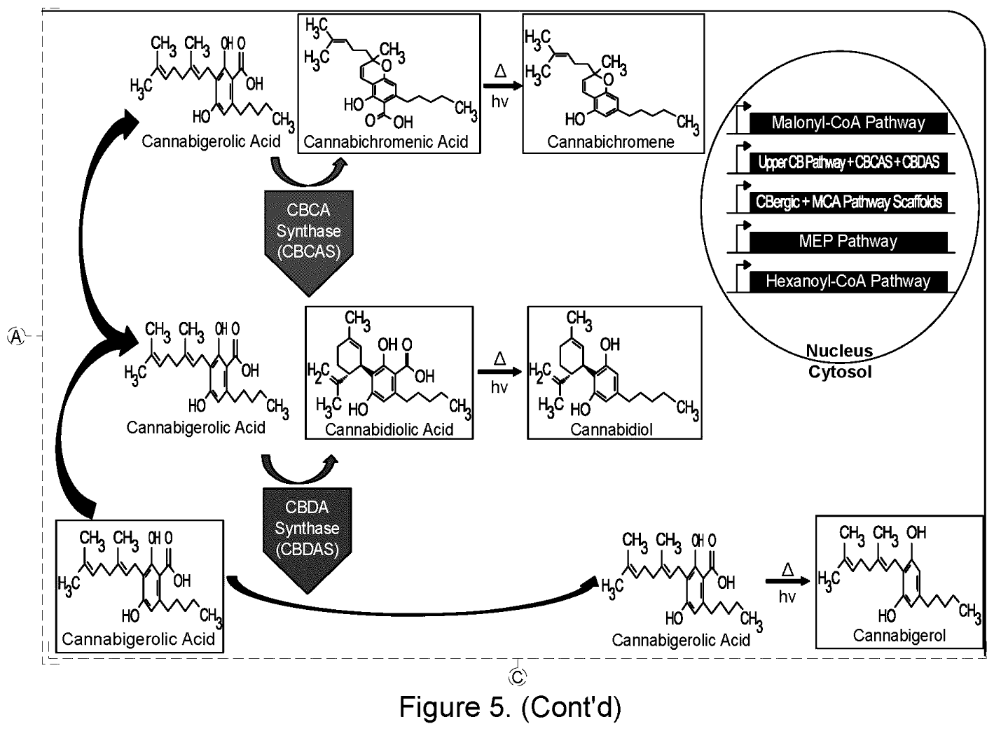

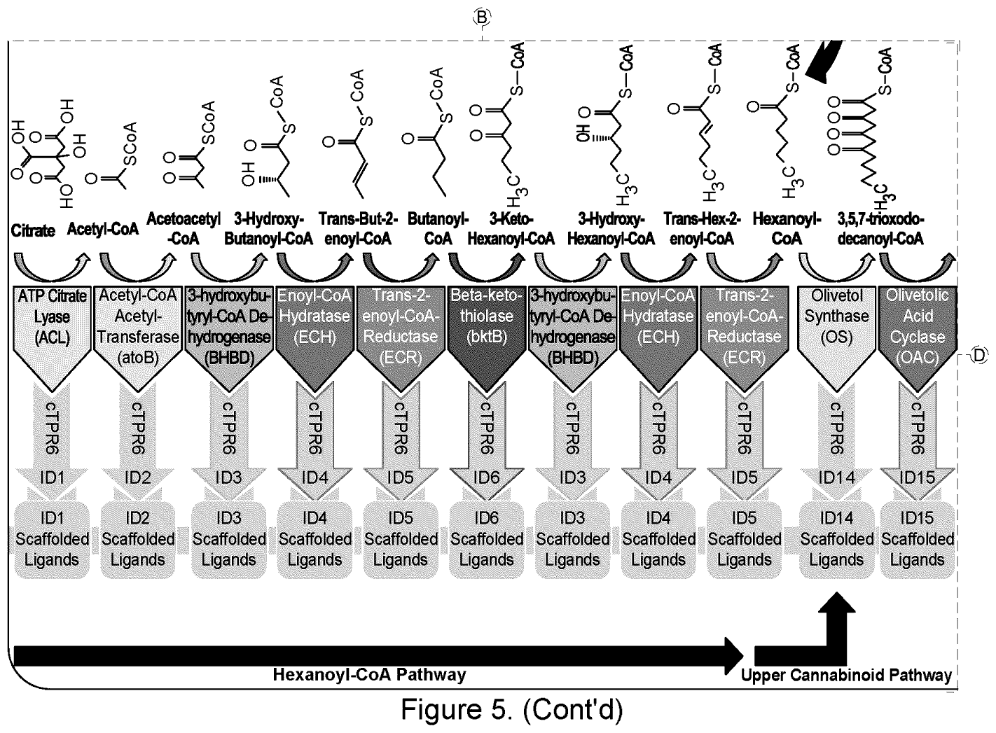

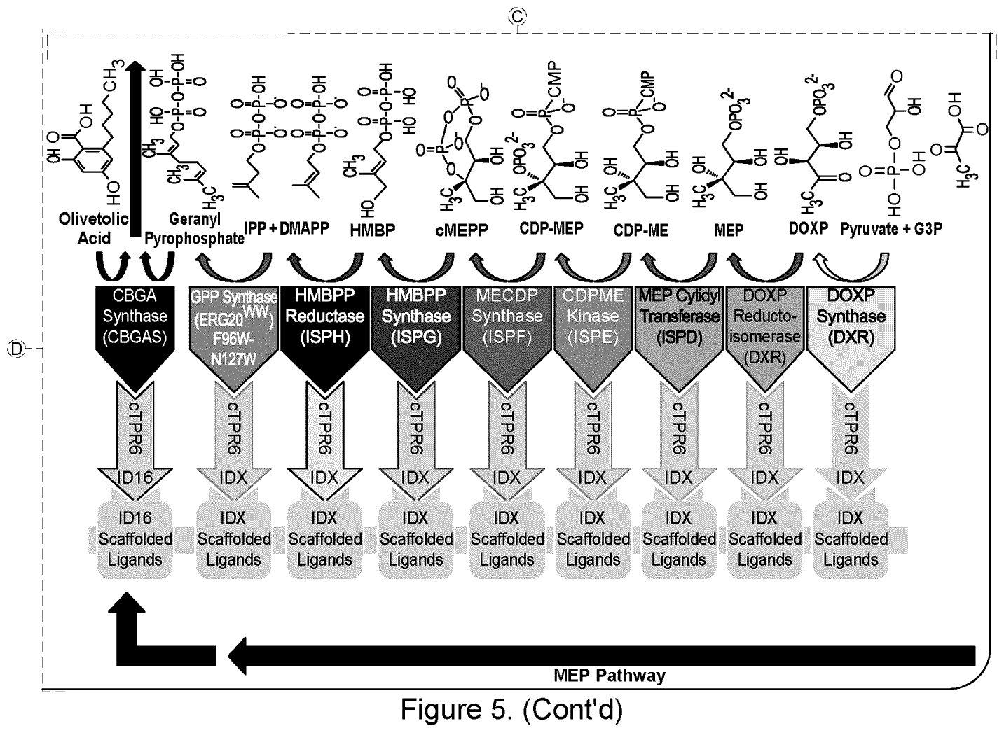

In some embodiments, the 2-C-methylerythritol 4-phosphate (MEP) pathway, which also can produce geranyl pyrophosphate, can be substituted for the scaffolded mevalonate pathway at the C-terminus of the scaffold or can be included in a soluble form in addition to the scaffolded mevalonate pathway. For example, as shown in FIG. 5, the C-terminus of the scaffold can include a 1-deoxy-D-xylulose-5-phosphate (DOXP) synthase, a DOXP reductoisomerase, a MEP cytidyl transferase, a 4-diphosphocytidyl-2-C-methylerythritol (CDPME) kinase, a 2-C-methyl-D-erythritol 2,4-cyclodiphosphate (MECDP) synthase, a 4-hydroxy-3-methyl-but-2-enyl pyrophosphate (HMBPP) synthase, a HMBPP reductase, and a GPPS. Pyruvate and glyceraldehyde-3-phosphate (G3P) can be used as substrates for DOXP-synthase-catalyzed DOXP synthesis. DOXP is classified under EC 2.2.1.7. DOXP can be the substrate for DOXP reductoisomerase (DXR)-catalyzed MEP synthesis. DXR is classified under EC 1.1.1.267. MEP can be the substrate for 2-C-methyl-D-erythritol 4-phosphate cytidylyltransferase (ISPD)-catalyzed 4-diphosphocytidyl-2-C-methylerythritol (CDP-ME) synthesis. ISPD is classified under EC 2.7.7.60. CDP-ME can be the substrate for 4-diphosphocytidyl-2-C-methyl-D-erythritol kinase (ISPE)-catalyzed 4-diphosphocytidyl-2-C-methyl-D-erythritol 2-phosphate (CDP-MEP) synthesis. ISPE is classified under EC 2.7.1.148. CDP-MEP can be the substrate for 2-C-methyl-D-erythritol 2,4-cyclodiphosphate synthase (ISPF)-catalyzed 2-C-methyl-D-erythritol 2,4-cyclodiphosphate (cMEPP) synthesis. ISPF is classified under EC 4.6.1.12. cMEPP can be the substrate for HMB-PP synthase (ISPG)-catalyzed (E)-4-Hydroxy-3-methyl-but-2-enyl pyrophosphate (HMBPP) synthesis. ISPG is classified under EC 1.17.7.1. HMBPP can be the substrate for 4-hydroxy-3-methylbut-2-enyl diphosphate reductase (ISPH)-catalyzed isopentenyl pyrophosphate (IPP) and dimethylallyl pyrophosphate (DMAPP) synthesis. ISPH is classified under EC 1.17.1.2. IPP and DMAPP can be substrates for GPPS-catalyzed geranyl pyrophosphate synthesis. GPPS is classified under EC 2.5.1.1.

In some embodiments, the mevalonate pathway can be substituted for the scaffolded MEP pathway at the C-terminus of the scaffold or can be included in a soluble form in addition to the scaffolded MEP pathway.

In the embodiment shown in FIG. 1A and FIG. 1B, a second multi-enzymatic scaffold can be co-expressed to enhance cytosolic titers of malonyl-CoA, another secondary substrate which can be used in cannabinoid biosynthesis. Such a scaffold can include an ATP citrate lyase (ACL) and acetyl-CoA carboxylase (ACC) in tandem. In some embodiments, the ACL and ACC are paired in duplicate or triplicate along the scaffold. If the ACL and ACC are paired in duplicate or triplicate, the two or three ACLs on the scaffold can be the same or different, and the two or three ACCs can be the same or different. In any of the embodiments, malonyl-CoA can be supplemented into the growth media instead of, or in addition to, being supplied by a scaffolded malonyl-CoA pathway.

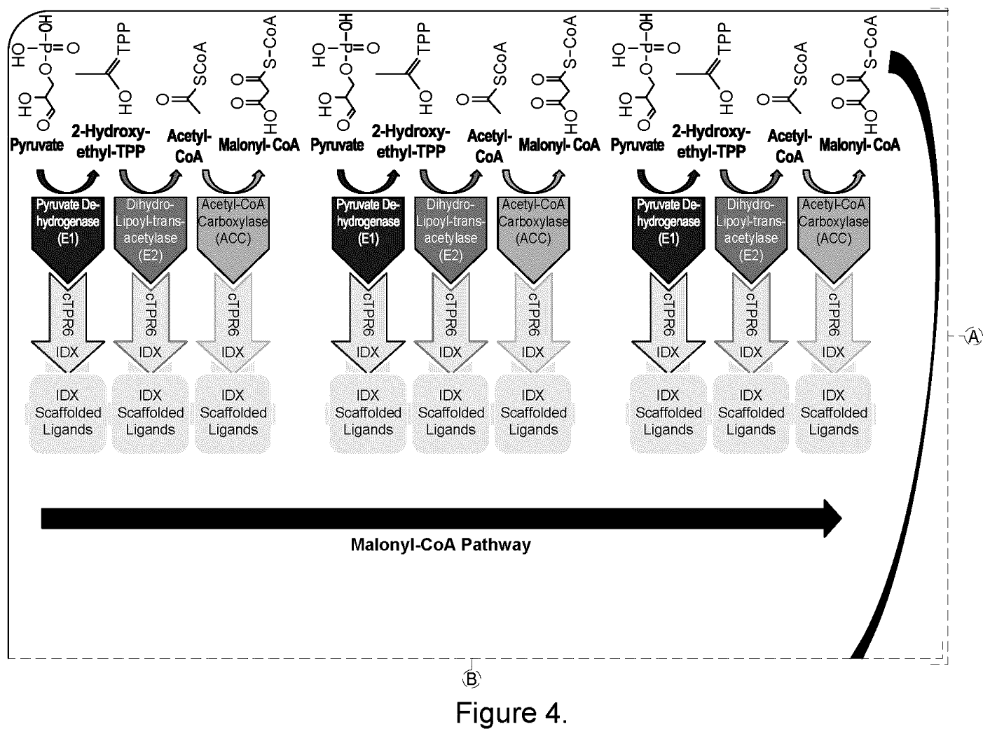

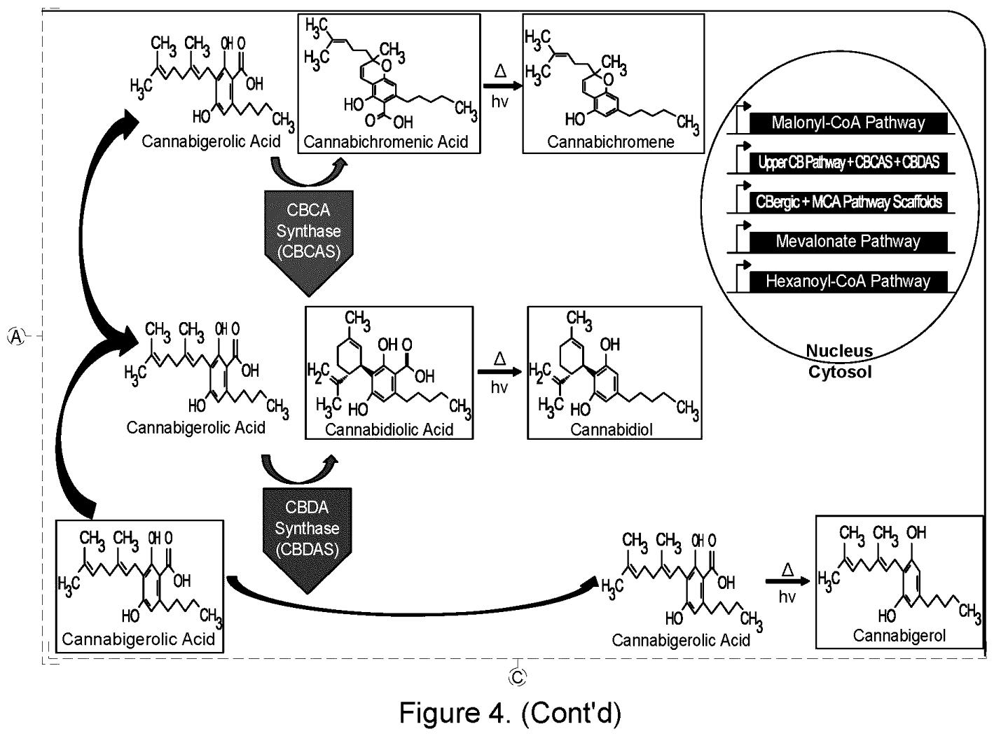

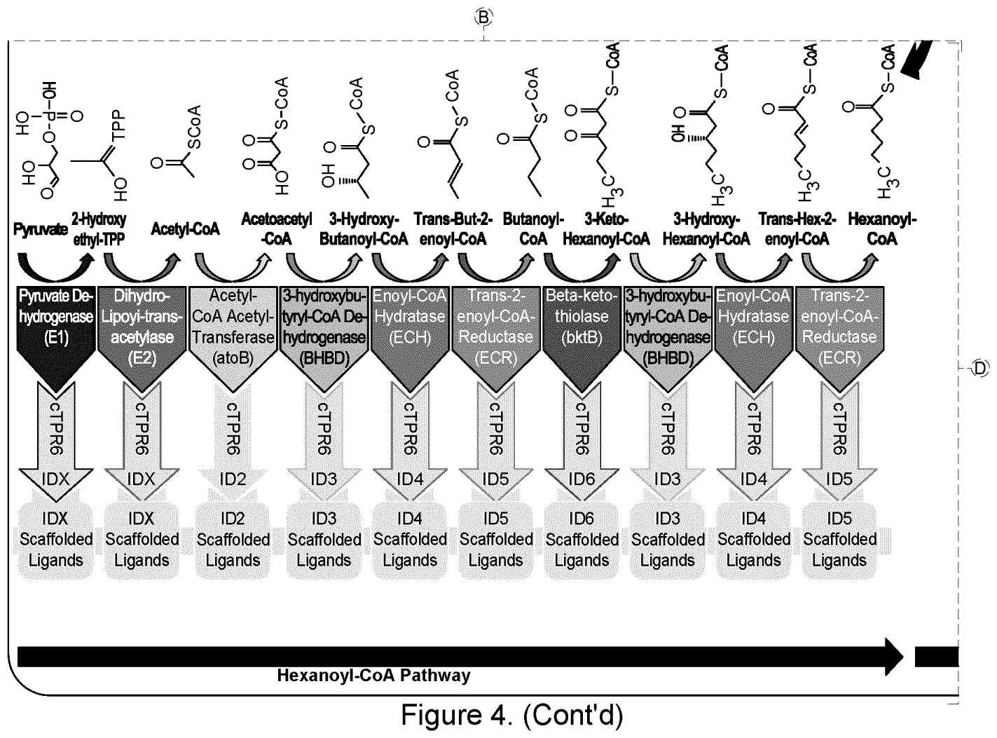

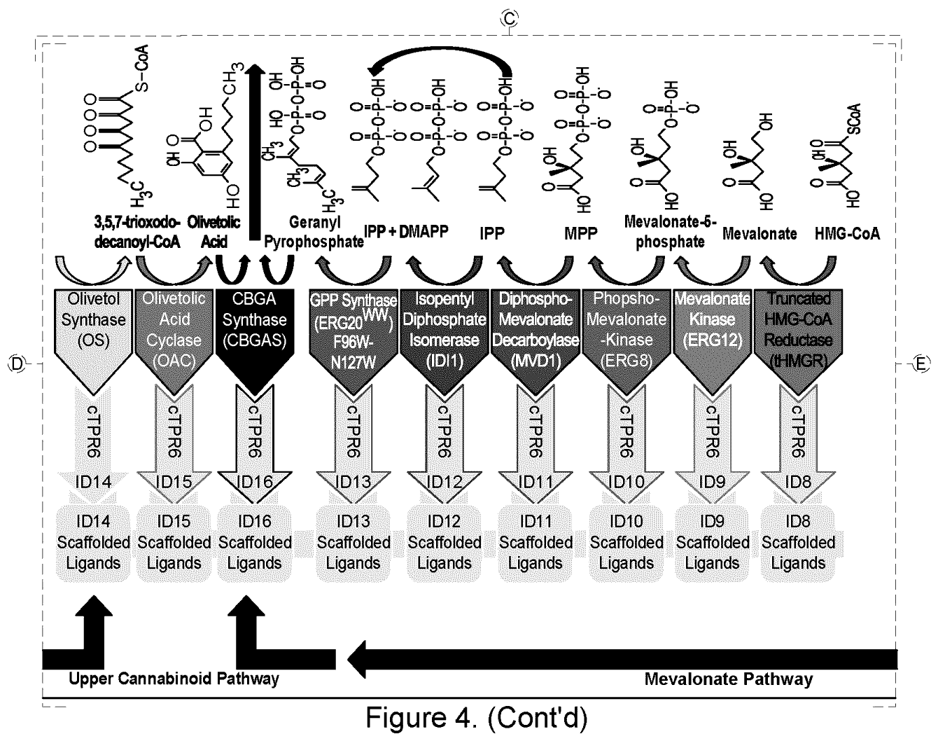

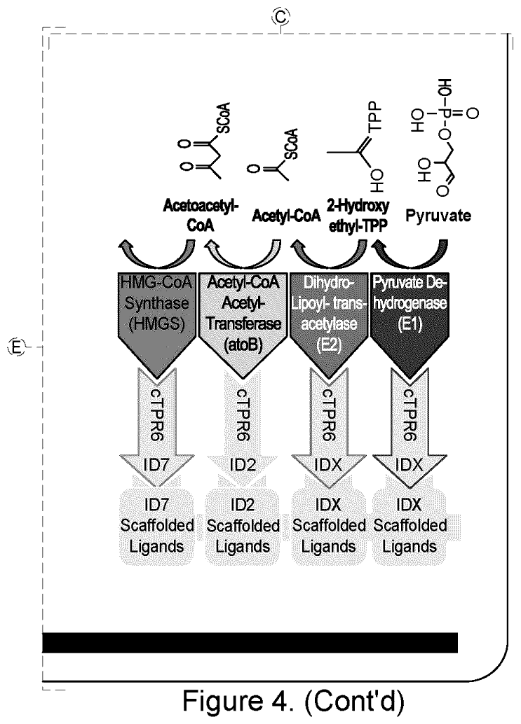

In any of the embodiments in which an ACL enzyme is used, a pyruvate dehydrogenase (E1) and a dihydrolipoyl transacetylase (E2) can be substituted for the ACL. For example, as shown in FIG. 4, a pyruvate dehydrogenase (E1) and a dihydrolipoyl transacetylase (E2) can be substituted upstream of scaffolded mevalonate, hexanoyl-CoA, and malonyl-CoA pathways. Using both a pyruvate dehydrogenase (E1) and a dihydrolipoyl transacetylase can allow acetyl-CoA to be produced using pyruvate rather than citrate as the primary substrate. In such embodiments, pyruvate also can be supplemented in the growth media. Pyruvate dehydrogenases and dihydrolipoyl transacetylases are constituents of the multi-enzyme pyruvate dehydrogenase complex that catalyze acetyl-CoA production from pyruvate. E1 and E2 are found in bacteria and eukaryotes.

As shown in FIG. 1A and FIG. 1B, the co-scaffolded upper cannabinoid pathway can include an olivetol synthase (OS), an olivetolic acid cyclase (OAC), and an aromatic prenyl-transferase (APT) such as a CBGA synthase (CBGAS). The upper cannabinoid pathway can begin using hexanoyl-CoA and three malonyl CoAs as the substrate for olivetol synthase-catalyzed 3,5,7-trioxododecanoyl-CoA synthesis. Olivetol synthase is classified under EC 2.3.1.206. 3,5,7-trioxododecanoyl-CoA can be used as a substrate for OAC-catalyzed olivetolic acid synthesis. OAC is classified under EC 4.4.1.26.

At the flux intersection of the converging N-terminal hexanoyl-CoA/upper cannabinoid and C-terminal mevalonate/MEP pathways (near the scaffold midpoint), an APT such as CBGAS can use olivetolic acid from the hexanoyl-CoA/upper cannabinoid pathways and geranyl pyrophosphate from the mevalonate or MEP pathway as substrates for cannabigerolate synthesis. A suitable APT is classified under EC 2.5.1.102.

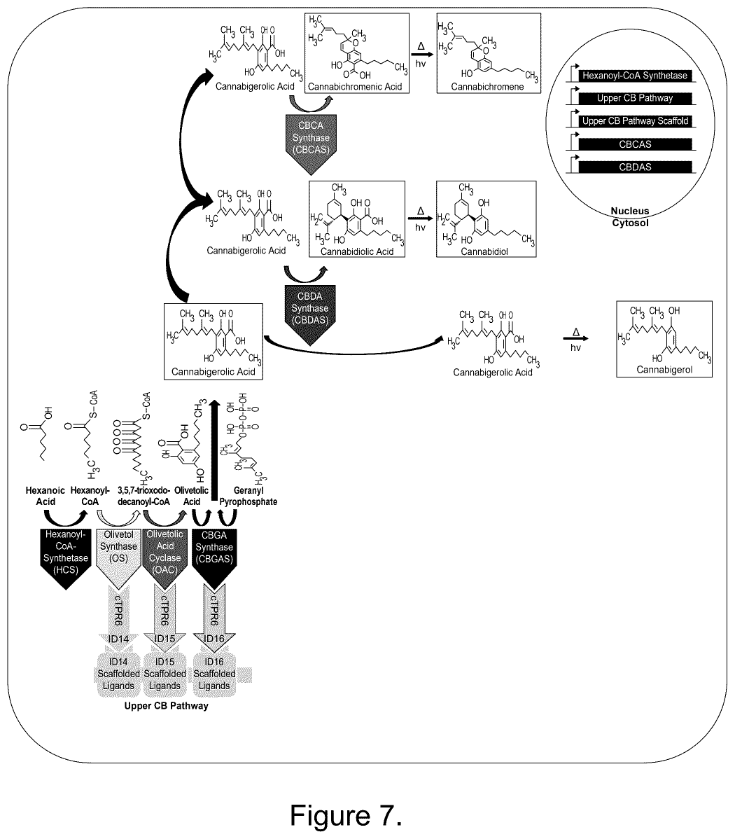

In some embodiments, enzymes in the upper cannabinoid pathway can be scaffolded with a hexanoyl-CoA synthetase (HCS) to biosynthesize cannabigerolate. In some embodiments, a soluble HCS can be used with scaffolded enzymes of the upper cannabinoid pathway to biosynthesize cannabigerolate as shown in FIG. 7. Suitable enzymes for the upper cannabinoid pathway are described above.

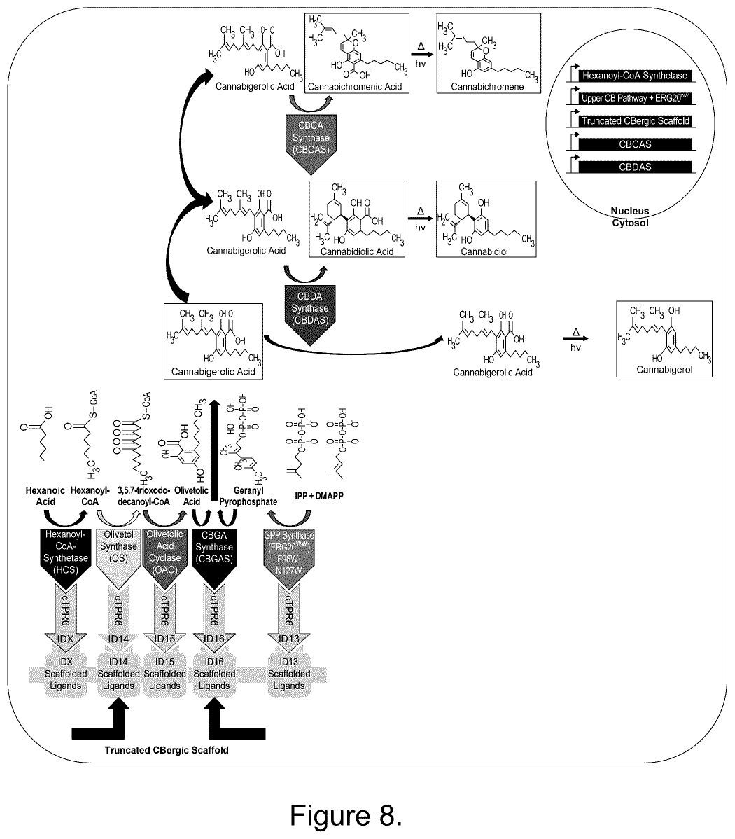

In some embodiments, a minimal bidirectional scaffold, such as the one depicted in FIG. 8, can be used in which HCS is on the N-terminus of the scaffold, a GPPS is on the C-terminus of the scaffold, and enzymes in the upper cannabinoid pathway are scaffolded between the HCS and GPPS.

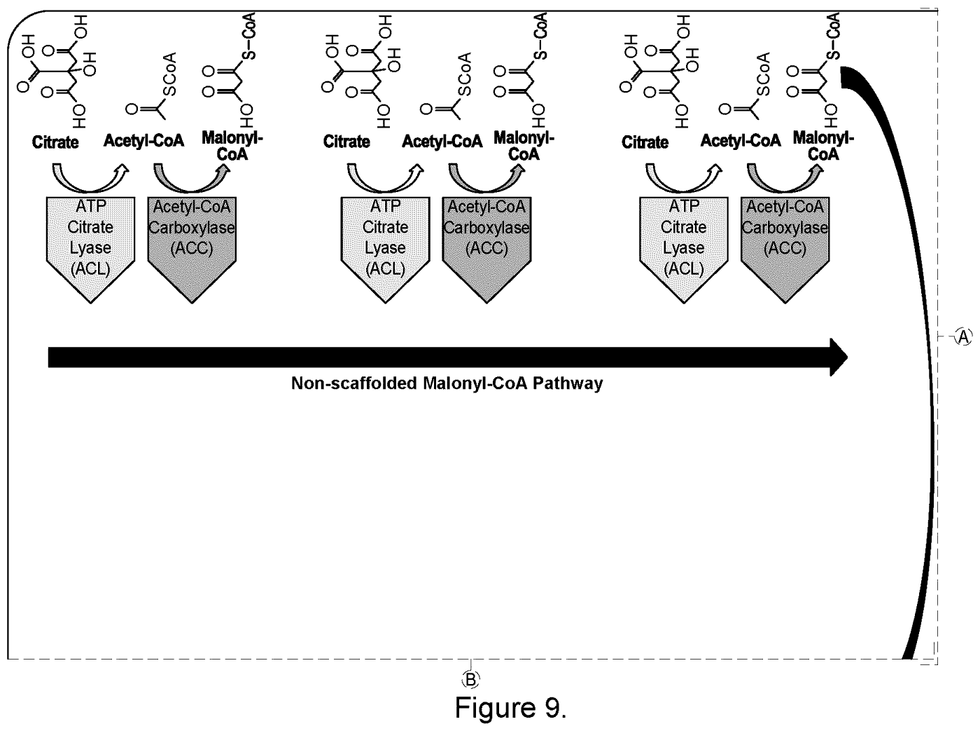

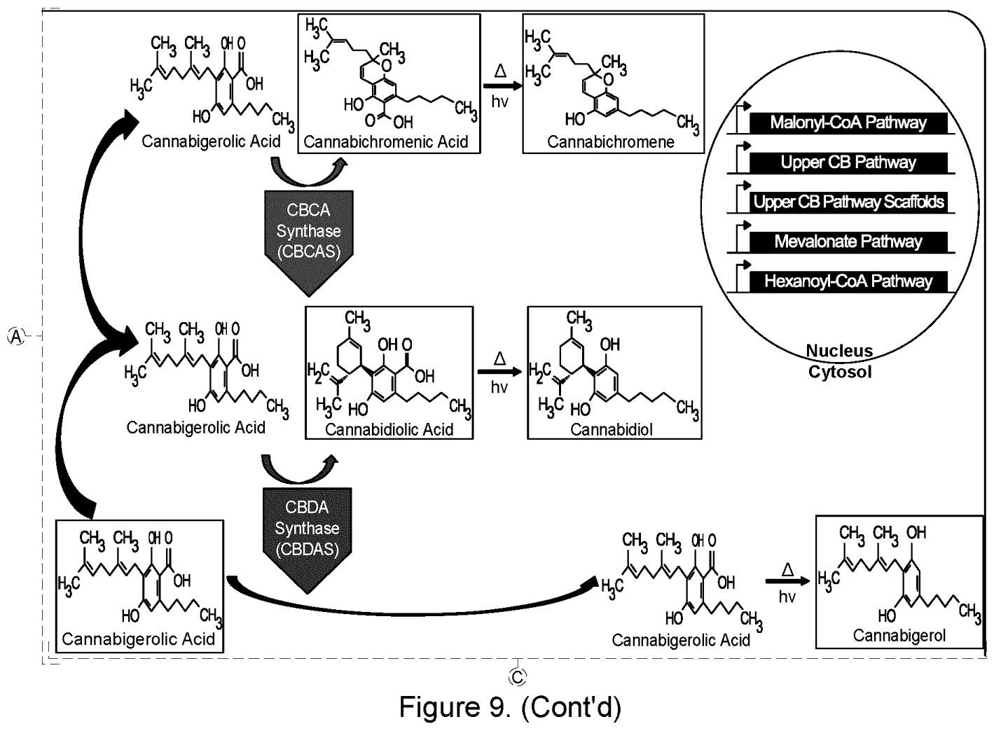

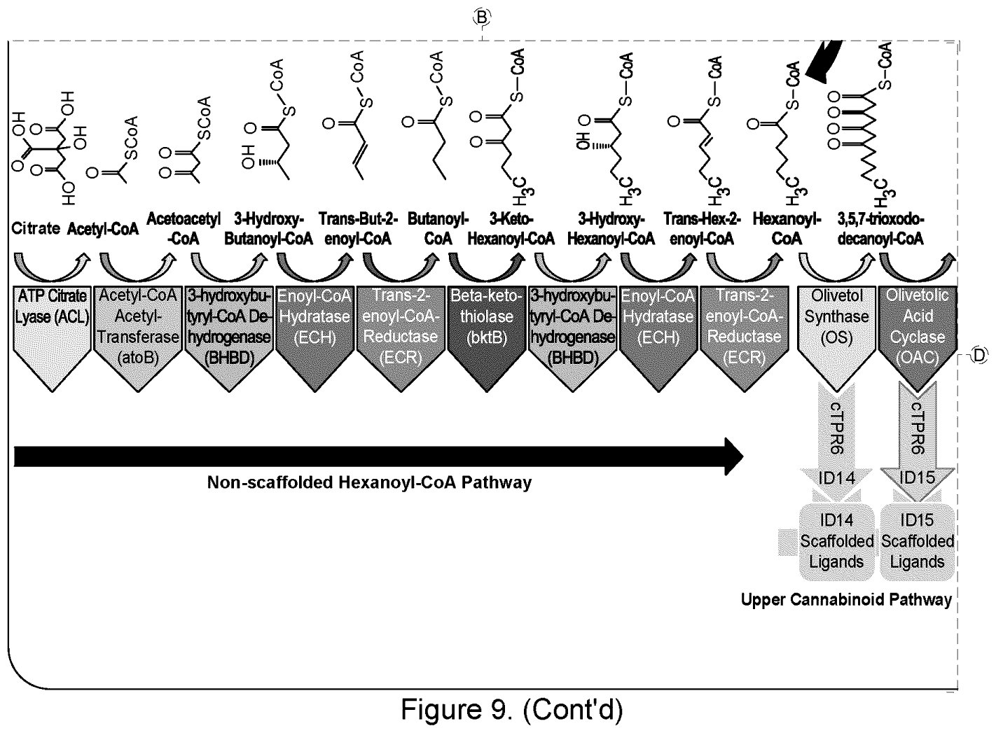

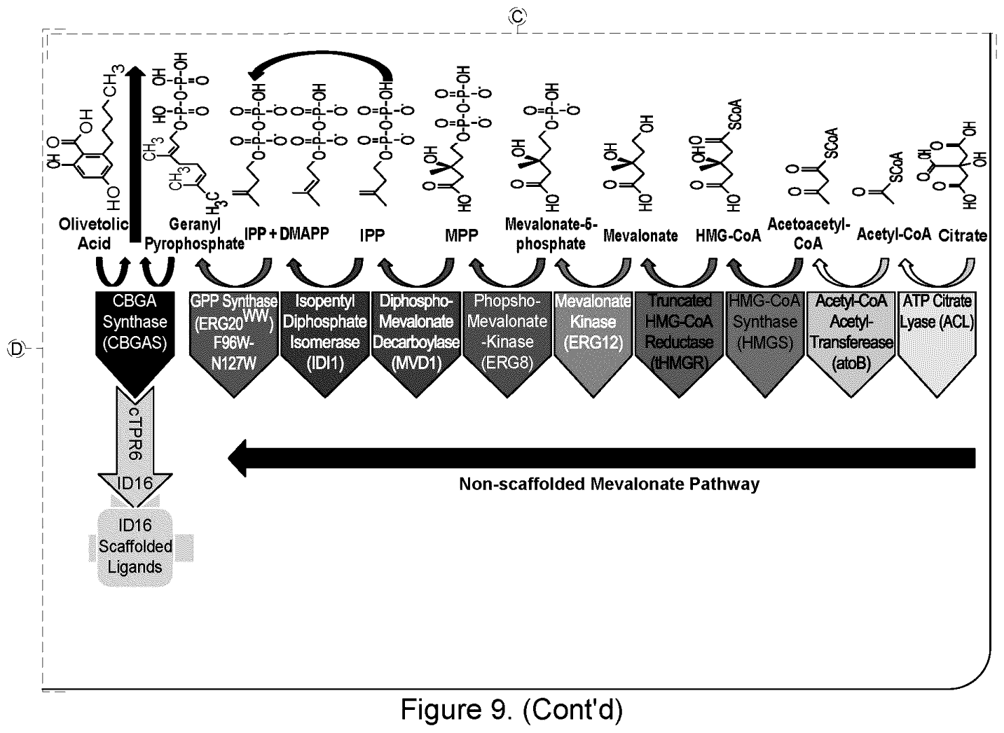

In some embodiments, such as the embodiment shown in FIG. 9, the enzymes in the upper cannabinoid pathway can be scaffolded, while the enzymes in the hexanoyl-CoA pathway, enzymes in the mevalonate pathway, and enzymes in the malonyl-CoA pathway can be soluble. In some embodiments, the enzymes in the upper cannabinoid pathway can be scaffolded, while the enzymes in the hexanoyl-CoA pathway, enzymes in the MEP pathway, and enzymes in the malonyl-CoA pathway can be soluble. In such embodiments, HCS can be substituted for the soluble forms of the enzymes of the hexanoyl-CoA pathway. Suitable enzymes for each of these pathways are described above.

In some embodiments, the enzymes in the upper cannabinoid pathway can be scaffolded, while a hexanoyl-CoA synthase, enzymes in the mevalonate or MEP pathway, and enzymes in the malonyl-CoA pathway can be soluble. Suitable enzymes for each of these pathways are described above.

In some embodiments, a HCS can be scaffolded N-terminally relative to the scaffolded enzymes in the upper cannabinoid pathway, while enzymes in the mevalonate or MEP pathway, and enzymes in the malonyl-CoA pathway can be soluble. Suitable enzymes for each of these pathways are described above.

In some embodiments, the enzymes in the upper cannabinoid pathway can be scaffolded, while the enzymes in the hexanoyl-CoA pathway or a hexanoyl-CoA synthase and enzymes in the mevalonate or MEP pathways can be soluble. In some embodiments, the enzymes in the hexanoyl-CoA pathway or a hexanoyl-CoA synthase can be scaffolded N-terminal to the enzymes in the upper cannabinoid pathway, and enzymes in the mevalonate or MEP pathways can be soluble. In such embodiments, malonyl-CoA can be supplemented. Suitable enzymes for each of these pathways are described above.

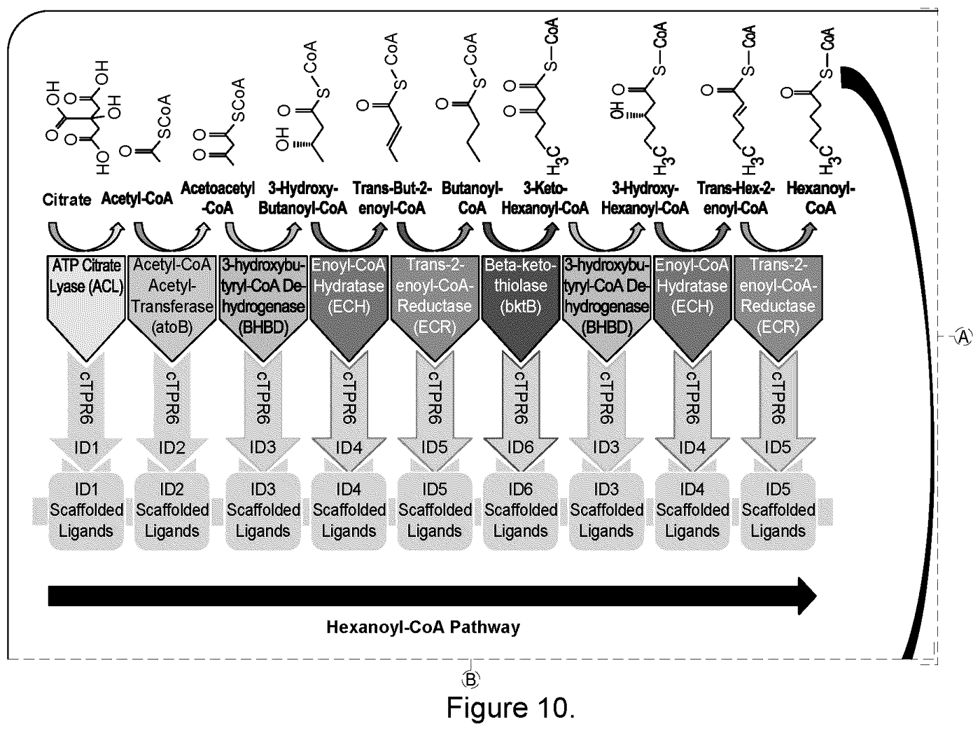

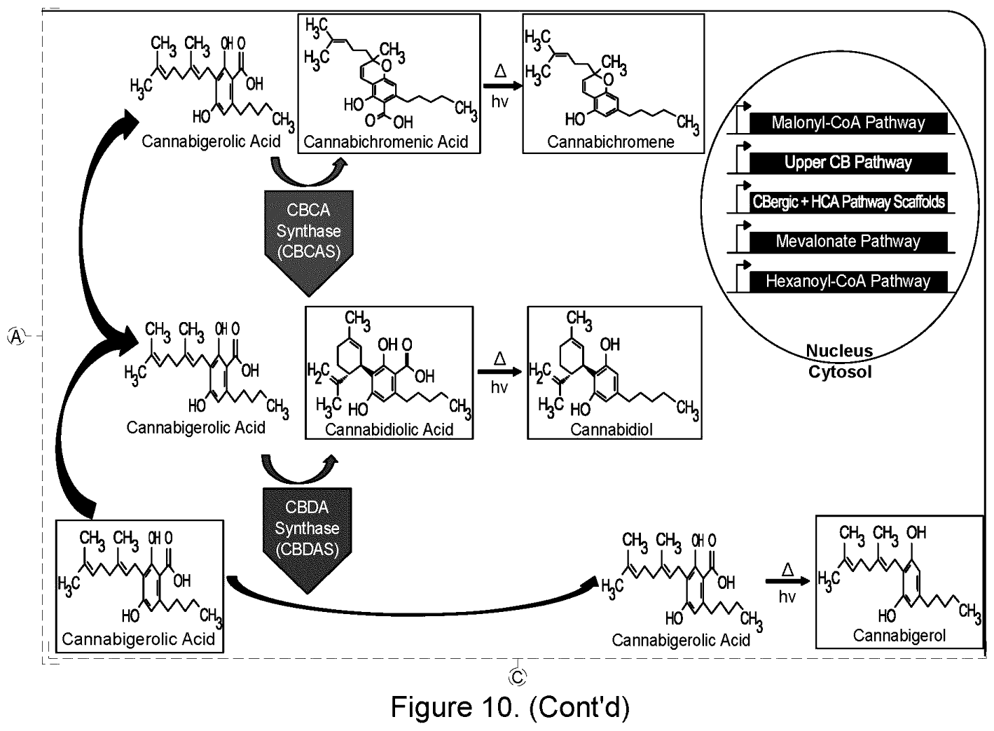

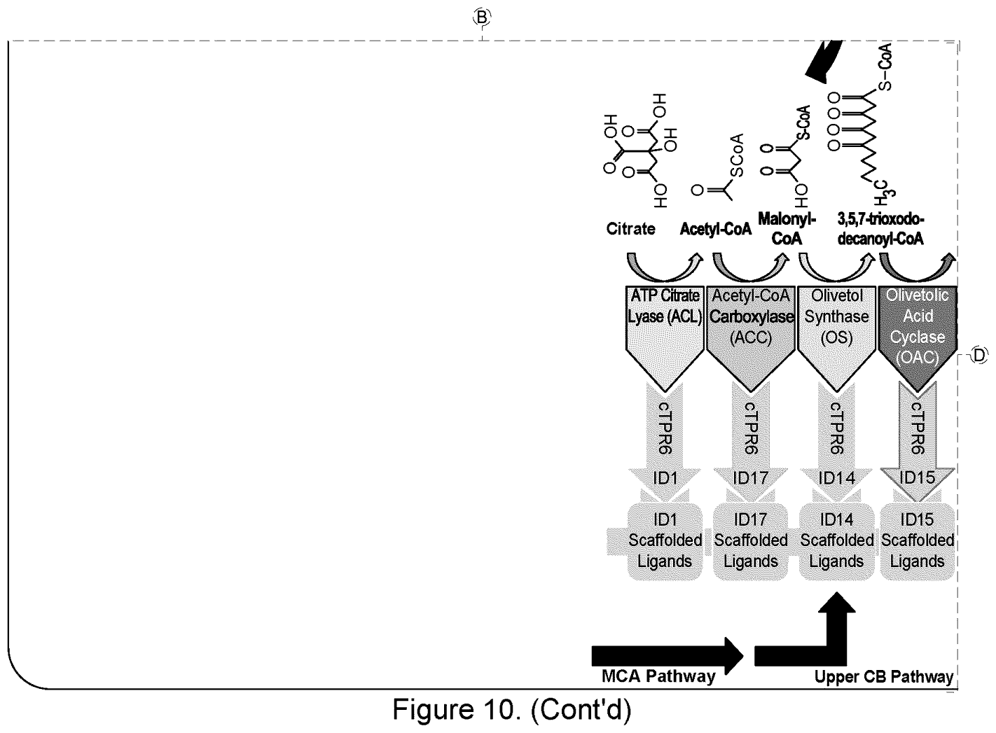

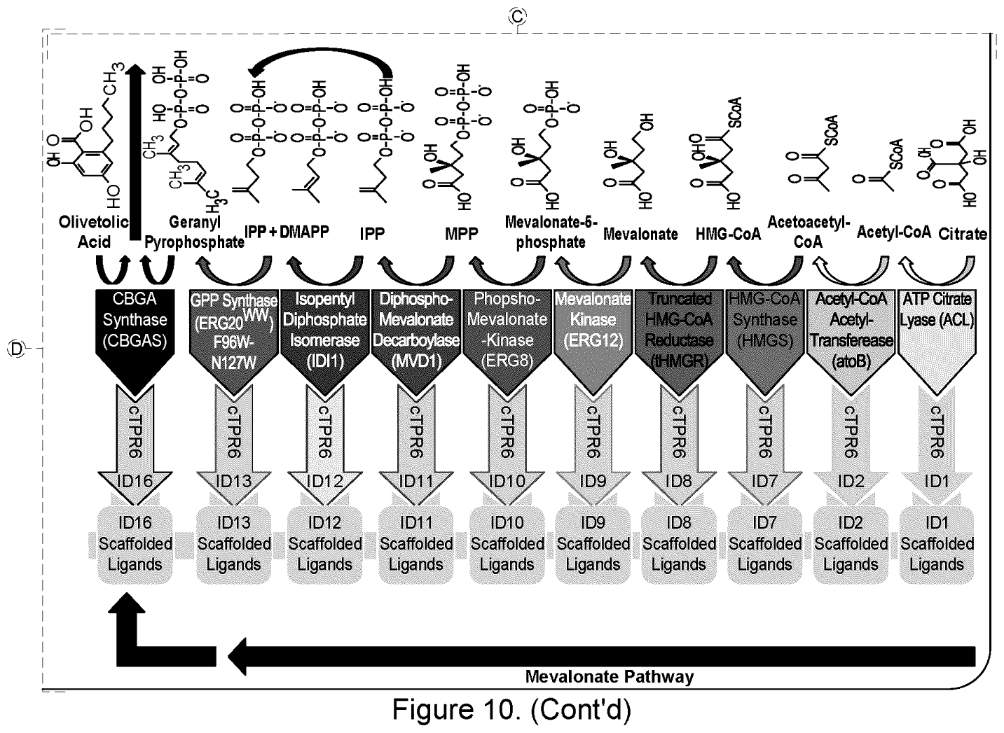

In some embodiments, such as the embodiment shown in FIG. 10, a bi-directional scaffold can include enzymes of the malonyl-CoA (MCA) pathway on the N-terminus of the scaffold, enzymes of the mevalonate pathway on the C-terminus of the scaffold, and enzymes in the upper cannabinoid pathway in between. In some embodiments, a bi-directional scaffold can include enzymes of the malonyl-CoA pathway on the N-terminus of the scaffold, enzymes of the MEP pathway on the C-terminus of the scaffold, and enzymes in the upper cannabinoid pathway in between. In such embodiments, enzymes of the hexanoyl-CoA pathway can be on a separate scaffold or can be soluble. In some embodiments, HCS can be substituted for scaffolded or soluble enzymes of the hexanoyl-CoA pathway.

In some embodiments, each of the pathways are on separate scaffolds. For example, in one embodiment, enzymes of the upper cannabinoid pathway can be on one scaffold, enzymes of the mevalonate or MEP pathway can be localized on one scaffold, enzymes of the hexanoyl-CoA pathway can be localized on one scaffold, and enzymes of the malonyl-CoA pathway can be localized on another scaffold.

Cannabigerolic acid biosynthesized in any of the embodiments described herein can be isolated and/or can be used as a substrate for synthesis of other secondary and tertiary cannabinoids using downstream cannabinoid synthases. In order to generate a more diverse profile of cannabinoids, the downstream cannabinoid synthases typically are not scaffolded, as scaffolding would favor production of the terminal cannabinoid. In some embodiments, however, one or more of the downstream cannabinoid synthases can be included on a scaffold described herein.

For example, one or more of cannabidiolic acid synthase (CBDAS), cannabichromenic acid synthase (CBCAS), tetrahydrocannabinolic acid synthase (THCAS), or other cannabinoid synthases can be used to produce additional cannabigerolate-derived cannabinoids. For example, a CBDAS; a CBCAS; a THCAS; a CBDAS and a CBCAS; a CBDAS and a THCAS; a CBCAS and a THCAS; or a CBDAS, CBCAS, and THCAS can be used to produce additional cannabigerolate-derived cannabinoids such as one or more of cannabiodiolic acid, cannabichromenic acid, and delta-9 tetrahydrocannabinolic acid. CBDAS is classified under EC 1.21.3.8 and can catalyze the synthesis of cannabidiolic acid from cannabigerolic acid. CBCAS is classified under EC 1.3.3- and can catalyze the synthesis of cannabichromenic acid from cannabigerolic acid. THCAS is classified under EC 1.21.3.7 and can catalyze the synthesis of delta-9 tetrahydrocannabinolic acid from cannabigerolic acid.

### Host Cells for Producing Cannabinoids

Cannabinoids can be produced in host cells or in vitro using a multi-enzymatic scaffold as described herein. Suitable host cells include any microorganism, eukaryotic or prokaryotic, such as bacteria (e.g., Escherichia coli, Bacillus, Brevibacterium, Streptomyces, or Pseudomonas), yeast (e.g., Pichia pastoris, Saccharomyces cerevisiae, Yarrowia lipolytica, Kluyveromyces marxianus, or Komagataella phaffii) and other fungi (e.g., Neurospora crassa), and green algae (e.g., Dunaliella sp., Chlorella variabilis, Euglena mutabilis, or Chlamydomonas reinhardtii), as well as plant cells (e.g., tobacco, Cannabis, or other photosynthetic plant cells) that can be maintained in culture or, in the case of plant cells such as those from tobacco or cannabis plants, can be engineered in culture and cultivated as intact transgenic plants. Such host cells or plant may or may not naturally produce cannabinoids.

A host cell can be modified to contain one or more exogenous nucleic acids that encode a scaffold as described herein and one or more exogenous nucleic acids that encode the engineered enzymes. The term “nucleic acid” as used herein encompasses both RNA and DNA, including cDNA, genomic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

The term “exogenous” as used herein with reference to nucleic acid and a particular host cell refers to any nucleic acid that does not originate from that particular host cell as found in nature. Thus, non-naturally-occurring nucleic acid is considered to be exogenous to a host cell once introduced into the host cell. It is important to note that non-naturally-occurring nucleic acid can contain nucleic acid sequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host cell, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid.

A nucleic acid that is naturally-occurring can be exogenous to a particular cell. For example, an entire chromosome isolated from a cell of organism X is an exogenous nucleic acid with respect to a cell of organism Y once that chromosome is introduced into Y's cell.

It is noted that a host cell can be given an exogenous nucleic acid molecule that encodes a polypeptide having an enzymatic activity that catalyzes the production of a compound not normally produced by that host cell. Alternatively, or additionally, a host cell can be given an exogenous nucleic acid molecule that encodes a polypeptide having an enzymatic activity that catalyzes the production of a compound that is normally produced by that host cell. In this case, the recombinant host cell can produce more of the compound, or can produce the compound more efficiently, than a similar host cell not having the genetic modification.

An enzyme having a particular enzymatic activity can be a polypeptide that is either naturally-occurring or non-naturally-occurring. A naturally-occurring polypeptide is any polypeptide having an amino acid sequence as found in nature, including wild-type and polymorphic polypeptides. Such naturally-occurring polypeptides can be obtained from any species including, without limitation, animal (e.g., mammalian), plant, fungal, and bacterial species. A non-naturally-occurring polypeptide is any polypeptide having an amino acid sequence that is not found in nature. Thus, a non-naturally-occurring polypeptide can be a mutated version of a naturally-occurring polypeptide, or an engineered polypeptide such as the engineered enzymes described herein that contain IDs. For example, a non-naturally-occurring polypeptide having geranyl pyrophosphate synthase activity can be a mutated version of a naturally-occurring polypeptide having geranyl pyrophosphate synthase activity. For example, the GPPS encoded by Erg20 may include a substitution of a tryptophan for phenylalanine at position 96 and a substitution of a tryptophan for asparagine at position 127 (referred to as Erg20^{WW}). Erg20^{WW} favors production of geranyl pyrophosphate over farnesyl pyrophosphate. See, Jiang, et al., Metab Eng. 2017, 41:57-66. For example, a truncated HMGR (tHMGR) such as an N-terminally truncated HMGR that includes the catalytic domain but not the transmembrane or regulatory domains of HMGR can be used. For example, the HMGR from A. thaliana (GenBank Accession No. J04537) or a HMGR from S. cerevisiae (which contains only residues 646-1025) can be truncated to remove the transmembrane and/or regulatory domains and used in a scaffold described herein to remove a bottleneck in the mevalonate pathway. HMGR catalyzes the rate-limiting step in the mevalonate pathway (see, e.g., Song et al., 2017, Scientific reports, doi:10.1038/s41598-017-15005-4). For example, the nucleic acid encoding an atoB from S. cerevisiae can be modified to contain a synthetic 5′ UTR (such as the synthetic 5′ UTR sequence: 5′-cggcacccctacaaacagaaggaatataaa-3′ (SEQ ID NO:82)) and can be used in the scaffold as it alters atoB expression to facilitate flux-rebalancing in favor of production of acetoacetyl-CoA over the reverse reaction product butyryl-CoA (see Kim et al., 2018, Bioresour Technol, doi: 10.1016/j.biortech.2017.10.014). A polypeptide can be mutated by, for example, sequence additions, deletions, substitutions, or combinations thereof.

Any of the enzymes described herein that can be used to produce one or more cannabinoids can have at least 70% sequence identity (e.g., at least 75%, 80%, 85%, 90%, 95%, 97%, 98%, 99%, or 100%) to the amino acid sequence of the corresponding wild-type enzyme. It will be appreciated that the sequence identity can be determined on the basis of the mature enzyme (e.g., with any signal sequence removed).

For example, an ACL can have at least 70% sequence identity (e.g., at least 75%, 80%, 85%, 90%, 95%, 97%, 98%, 99%, or 100%) to the amino acid sequence of a Homo sapiens ACL (see SEQ ID NO:83, FIG. 6A), or an ACL from Rattus norvegicus, Mus musculus, or Ciona intestinalis, e.g., GenBank Accession Nos. AAA74463, AAK56081, and BAB00624, respectively.

For example, an acetyl-CoA acetyltransferase (atoB) can have at least 70% sequence identity (e.g., at least 75%, 80%, 85%, 90%, 95%, 97%, 98%, 99%, or 100%) to the amino acid sequence of an Escherichia coli atoB (see SEQ ID NO:84, FIG. 6A), or an atoB from Cupriavidus necator, Clostridium acetobutylicum, or Arabidopsis thaliana, e.g., GenBank Accession Nos. CAJ92573, AAK80816, and AAM67058, respectively. In some embodiments, a malonyl-CoA acyl carrier protein transacylase from Saccharomyces cerevisiae, Homo sapiens, Serratia plymuthica, or Dickeya paradisiaca can be substituted for atoB, e.g., GenBank Accession Nos. DAA10992, AAH30985, AGO55277, and ACS85236, respectively.

For example, a 3-hydroxy-butyryl-CoA dehydrogenase (BHBD) can have at least 70% sequence identity (e.g., at least 75%, 80%, 85%, 90%, 95%, 97%, 98%, 99%, or 100%) to the amino acid sequence of a Clostridium acetobutylicum BHBD (see SEQ ID NO:85, FIG. 6A), or a BHBD from Escherichia coli, Treponema denticola, or Arabidopsis thaliana, e.g., GenBank Accession Nos. AIZ91493, AAS11105, and AAN17431, respectively.

For example, an enoyl-CoA hydratase (ECH) can have at least 70% sequence identity (e.g., at least 75%, 80%, 85%, 90%, 95%, 97%, 98%, 99%, or 100%) to the amino acid sequence of a Clostridium acetobutylicum ECH (see SEQ ID NO:86, FIG. 6A), or an ECH from Acinetobacter oleivorans, Cupriavidus necator, or Acinetobacter baumannii, e.g., GenBank Accession Nos. ADI91469, CAJ91294, and ACJ57023, respectively.

For example, a beta-ketothiolase (bktB) can have at least 70% sequence identity (e.g., at least 75%, 80%, 85%, 90%, 95%, 97%, 98%, 99%, or 100%) to the amino acid sequence of a Cupriavidus necator bktB (see SEQ ID NO:87, FIG. 6A), or a bktB from Escherichia coli, Lactobacillus casei, or Clostridium acetobutylicum, e.g., GenBank Accession Nos. ALI39443, CAQ67083, and AAK80816, respectively.

For example, a trans-2-enoyl-CoA-reductase (ECR) can have at least 70% sequence identity (e.g., at least 75%, 80%, 85%, 90%, 95%, 97%, 98%, 99%, or 100%) to the amino acid sequence of a Treponema denticola ECR (see SEQ ID NO:88, FIG. 6A), or an ECR from Cupriavidus necator, Saccharomyces cerevisiae, or Klebsiella michiganensis, e.g., GenBank Accession Nos. AAP86010, DAA07148, and AIE72439, respectively.

For example, a hexanoyl-CoA synthetase (HCS), which is a type of acyl-activating enzyme (AAE), can have at least 70% sequence identity (e.g., at least 75%, 80%, 85%, 90%, 95%, 97%, 98%, 99%, or 100%) to the amino acid sequence of a C. sativa AAE1 (see SEQ ID NO:89, FIG. 6A, GenBank Accession No. AFD33345) or C. sativa AAE3 (GenBank Accession No. AFD33347). The C. sativa AAE1 and AAE3 each can use hexanoate as a substrate. See, Stout, et al., Plant J., 71(3): 353-365 (2012). In some embodiments, the AAE encoded by CsAAE1 can be used. See, GenBank Accession No. JN717233 for the coding sequence. In some embodiments, the AAE encoded by CsAAE3 can be used. See, GenBank Accession No. JN717233 for the coding sequence. In some embodiments, both CsAAE1 and CsAAE3 can be used.

For example, an HMG-CoA synthase (HMGS) can have at least 70% sequence identity (e.g., at least 75%, 80%, 85%, 90%, 95%, 97%, 98%, 99%, or 100%) to the amino acid sequence of a S. cerevisiae HMGS (see SEQ ID NO:90, FIG. 6A), or an HMGS from Arabidopsis thaliana, Lactobacillus casei, or Homo sapiens, e.g., GenBank Accession Nos. AEE83052, CAQ67081, and AAA62411, respectively.

For example, an HMG-CoA reductase (HMGR), N-terminally truncated or canonical, can have at least 70% sequence identity (e.g., at least 75%, 80%, 85%, 90%, 95%, 97%, 98%, 99%, or 100%) to the amino acid sequence of a S. cerevisiae HMGS (see SEQ ID NO:91, FIG. 6A), or an HMGR from Arabidopsis thaliana, Lactobacillus casei, or Homo sapiens, e.g., GenBank Accession Nos. AEE35849, CAQ67082, and AAA52679, respectively.

For example, a mevalonate kinase can have at least 70% sequence identity (e.g., at least 75%, 80%, 85%, 90%, 95%, 97%, 98%, 99%, or 100%) to the amino acid sequence of a S. cerevisiae mevalonate kinase (see SEQ ID NO:92, FIG. 6A), or a mevalonate kinase from Arabidopsis thaliana, Lactobacillus casei, or Homo sapiens, e.g., GenBank Accession Nos. AAD31719, CAQ66794, and AAF82407, respectively.

For example, a phosphomevalonate kinase can have at least 70% sequence identity (e.g., at least 75%, 80%, 85%, 90%, 95%, 97%, 98%, 99%, or 100%) to the amino acid sequence of a S. cerevisiae phosphomevalonate kinase (see SEQ ID NO:93, FIG. 6A), or a mevalonate kinase from Scheffersomyces stipitis, Lactobacillus casei, or Homo sapiens, e.g., GenBank Accession Nos. EAZ63544, CAQ66339, and AAH06089, respectively.

For example, a diphosphomevalonate decarboxylase can have at least 70% sequence identity (e.g., at least 75%, 80%, 85%, 90%, 95%, 97%, 98%, 99%, or 100%) to the amino acid sequence of a S. cerevisiae diphosphomevalonate decarboxylase (see SEQ ID NO:94, FIG. 6A), or a diphosphomevalonate decarboxylase from Arabidopsis thaliana, Lactobacillus casei, or Homo sapiens, e.g., GenBank Accession Nos. AAC67348, CAQ66795, and AAC50440, respectively.

For example, an isopentyl diphosphate isomerase can have at least 70% sequence identity (e.g., at least 75%, 80%, 85%, 90%, 95%, 97%, 98%, 99%, or 100%) to the amino acid sequence of a S. cerevisiae isopentyl diphosphate isomerase (see SEQ ID NO:95, FIG. 6A), or an isopentyl diphosphate isomerase from Arabidopsis thaliana, Lactobacillus casei, or Homo sapiens, e.g., GenBank Accession Nos. AAC49920, CAQ66796, and AAP35407, respectively.

For example, a geranyl pyrophosphate synthase (GPPS) (also known as a geranyl-diphospate synthase) can have at least 70% sequence identity (e.g., at least 75%, 80%, 85%, 90%, 95%, 97%, 98%, 99%, or 100%) to the amino acid sequence of the S. cerevisiae GPS or a GPPS from Acinetobacter baumannii, Lactobacillus casei, or Homo sapiens, e.g., GenBank Accession Nos. ACJ56139, CAQ66932, and AAH10004, respectively. In some embodiments, a mutant GPPS can be used. For example, the GPPS encoded by Erg20 may include a substitution of a tryptophan for phenylalanine at position 96 and a substitution of a tryptophan for asparagine at position 127 (referred to as Erg20^{WW}) (see SEQ ID NO:96, FIG. 6A). Erg20^{WW} favors production of geranyl pyrophosphate over farnesyl pyrophosphate. See, Jiang, et al., Metab Eng. 2017 41:57-66. In some cases, substituting a glutamic acid for lysine at position 179 of Erg20 (Erg20^{K179E}) can be used to produce a GPPS that favors production of geranyl pyrophosphate. See, WO2016010827A1.

For example, a DOXP synthase can have at least 70% sequence identity (e.g., at least 75%, 80%, 85%, 90%, 95%, 97%, 98%, 99%, or 100%) to the amino acid sequence of an Escherichia coli, Clostridium acetobutylicum, Treponema denticola, or Arabidopsis thaliana DOXP synthase, e.g., GenBank Accession Nos. CDH63925, AAK80036, AAS12424, and ANM65835, respectively.

For example, a DOXP reductoisomerase can have at least 70% sequence identity (e.g., at least 75%, 80%, 85%, 90%, 95%, 97%, 98%, 99%, or 100%) to the amino acid sequence of an Escherichia coli, Clostridium acetobutylicum, Treponema denticola, or Arabidopsis thaliana DOXP reductoisomerase, e.g., GenBank Accession Nos. CDH63708, AAK79760, AAS12860, and AAM61343, respectively.

For example, a MEP cytidyl transferase can have at least 70% sequence identity (e.g., at least 75%, 80%, 85%, 90%, 95%, 97%, 98%, 99%, or 100%) to the amino acid sequence of an Escherichia coli, Clostridium acetobutylicum, Treponema denticola, or Arabidopsis thaliana MEP cytidyl transferase, e.g., GenBank Accession Nos. CDH66380, AAK81121, AAS12810, and BAB21592, respectively.

For example, a CDPME kinase can have at least 70% sequence identity (e.g., at least 75%, 80%, 85%, 90%, 95%, 97%, 98%, 99%, or 100%) to the amino acid sequence of an Escherichia coli, Clostridium acetobutylicum, Treponema denticola, or Arabidopsis thaliana CDPME kinase, e.g., GenBank Accession Nos. CDH64802, AAK80844, AAS11855, and AEC07908, respectively.

For example, a MECDP synthase can have at least 70% sequence identity (e.g., at least 75%, 80%, 85%, 90%, 95%, 97%, 98%, 99%, or 100%) to the amino acid sequence of an Escherichia coli, Nicotiana tabacum, Treponema denticola, or Acinetobacter baumannii MECDP synthase, e.g., GenBank Accession Nos. CDH66379, AHM22925, AAS12811, and ACJ59227, respectively.

For example, an HMBPP synthase can have at least 70% sequence identity (e.g., at least 75%, 80%, 85%, 90%, 95%, 97%, 98%, 99%, or 100%) to the amino acid sequence of an Escherichia coli, Acinetobacter baumannii, Treponema denticola, or Arabidopsis thaliana HMBPP synthase, e.g., GenBank Accession Nos. AAN81487, ACJ58210, AAS11783, and AED97354, respectively.

For example, an HMBPP reductase can have at least 70% sequence identity (e.g., at least 75%, 80%, 85%, 90%, 95%, 97%, 98%, 99%, or 100%) to the amino acid sequence of an Escherichia coli, Acinetobacter baumannii, Treponema denticola, or Arabidopsis thaliana HMBPP reductase, e.g., GenBank Accession Nos. CDH63564, ACJ57384, AAS11585, and AEE86362, respectively.

For example, an acetyl-CoA carboxylase (ACC) can have at least 70% sequence identity (e.g., at least 75%, 80%, 85%, 90%, 95%, 97%, 98%, 99%, or 100%) to the amino acid sequence of a S. cerevisiae acetyl-CoA carboxylase (see SEQ ID NO:97, FIG. 6A), or an acetyl-CoA carboxylase from Homo sapiens, Treponema denticola, or Cupriavidus necator, e.g., GenBank Accession Nos. AAP94122, AAS11086, and CAQ67359, respectively.

For example, a pyruvate dehydrogenase (E1) and dihydrolipoyl transacetylase (E2) can have at least 70% sequence identity (e.g., at least 75%, 80%, 85%, 90%, 95%, 97%, 98%, 99%, or 100%) to the amino acid sequence of a Saccharomyces cerevisiae, Escherichia coli, Clostridium acetobutylicum, or Cupriavidus necator E1 and E2, e.g., GenBank Accession Nos. DAA07337, AMC97367, CAQ66617, and CAJ92510 for E1, and DAA10474, AUG14916, CAQ66619, and CAJ92511 for E2, respectively.

For example, an olivetol synthase (OS) can have at least 70% sequence identity (e.g., at least 75%, 80%, 85%, 90%, 95%, 97%, 98%, 99%, or 100%) to the amino acid sequence of an OS from C. sativa set forth in SEQ ID NO:98 (FIG. 6A) or the OS from C. sativa having GenBank Accession No. BAG14339. See, for example, Taura, et al., FEBS Letters 583 (2009) 2061-2066.

For example, an olivetolic acid cyclase (OAC) can have at least 70% sequence identity (e.g., at least 75%, 80%, 85%, 90%, 95%, 97%, 98%, 99%, or 100%) to the amino acid sequence of an OAC from C. sativa set forth in SEQ ID NO:99 (FIG. 6A) or the OAC from C. sativa having GenBank Accession No. AFN42527. See, for example, Gagne, et al., Proc. Natl. Acad. Sci. USA, 2012 109 (31) 12811-12816.

For example, a CBGAS can have at least 70% sequence identity (e.g., at least 75%, 80%, 85%, 90%, 95%, 97%, 98%, 99%, or 100%) to the amino acid sequence of an aromatic prenyl-transferase (APT) from Cannabis sativa such as the CBGAS set forth in SEQ ID NO:100 (FIG. 6A). See, for example, U.S. Patent Publication No. 20120144523A1 and U.S. Pat. No. 8,884,100B2. In some embodiments, a soluble APT from Streptomyces (e.g., NphB) can be used. See, for example, Carvalho et al., FEMS Yeast Research, 17, 2017, fox037.

For example, a cannabidiolic acid synthase (CBDAS) can have at least 70% sequence identity (e.g., at least 75%, 80%, 85%, 90%, 95%, 97%, 98%, 99%, or 100%) to the amino acid sequence of a CBDAS from C. sativa set forth in SEQ ID NO:101 (FIG. 6A) or the amino acid sequence of a CBDAS from C. sativa having GenBank Accession No. BAF65033. See, for example, Taura, et al., FEBS Lett. 581 (16), 2929-2934 (2007).

For example, a cannabichromenic acid synthase (CBCAS) can have at least 70% sequence identity (e.g., at least 75%, 80%, 85%, 90%, 95%, 97%, 98%, 99%, or 100%) to the amino acid sequence of a CBCAS from C. sativa set forth in SEQ ID NO:102 (FIG. 6A) or the amino acid sequence of a CBCAS from C. sativa as set forth in SEQ ID NO:2 of WO 2015/196275 A1. SEQ ID NO:2 of WO 2015/196275 A1 includes an N-terminal 28 amino acid signal peptide. All or a portion of the signal peptide can be removed from the sequence. The CBDAS from C. indica or C. ruderalis also can be used. In some embodiments, an Escherichia coli or yeast optimized nucleic acid sequence encoding a C. sativa CBCAS as set forth in SEQ ID NOs: 8 and 9, respectively, of WO 2015/196275 A1 can be used.

For example, a tetrahydrocannabinolic acid synthase (THCAS) can have at least 70% sequence identity (e.g., at least 75%, 80%, 85%, 90%, 95%, 97%, 98%, 99%, or 100%) to the amino acid sequence of a THCAS from C. sativa having GenBank Accession No. BAC41356. See, for example, Sirikantaramas, et al., J. Biol. Chem. 279 (38), 39767-39774 (2004).

The percent identity (homology) between two amino acid sequences can be determined as follows. First, the amino acid sequences are aligned using the BLAST 2 Sequences (B12seq) program from the stand-alone version of BLASTZ containing BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq-i c:\seq1.txt-j c:\seq2.txt-p blastp-o c:\output.txt. If the two compared sequences share homology (identity), then the designated output file will present those regions of homology as aligned sequences. If the two compared sequences do not share homology (identity), then the designated output file will not present aligned sequences. Similar procedures can be following for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

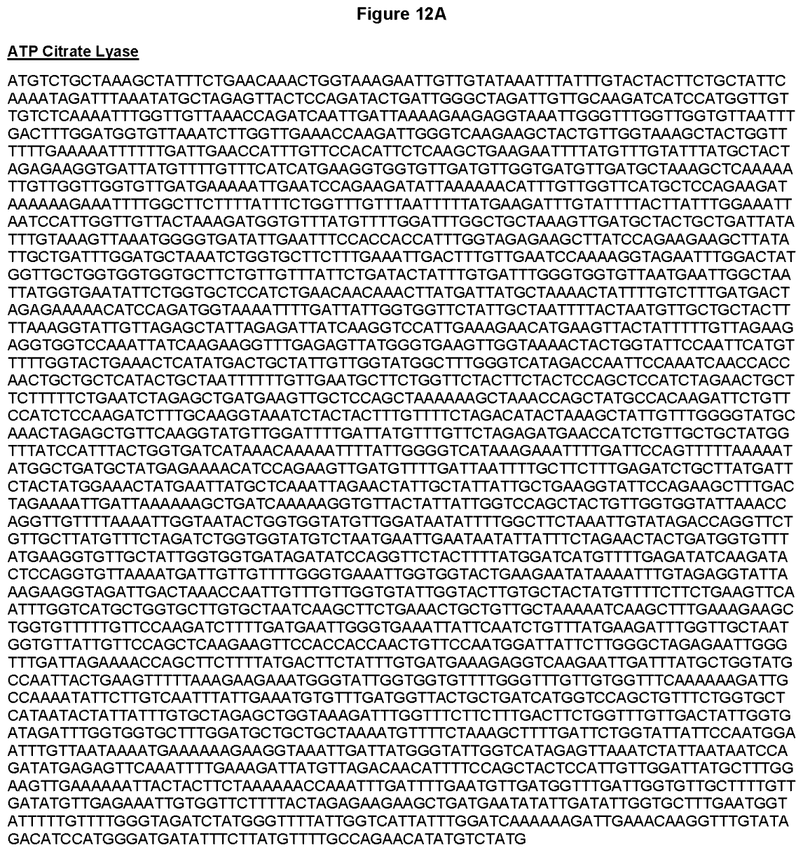

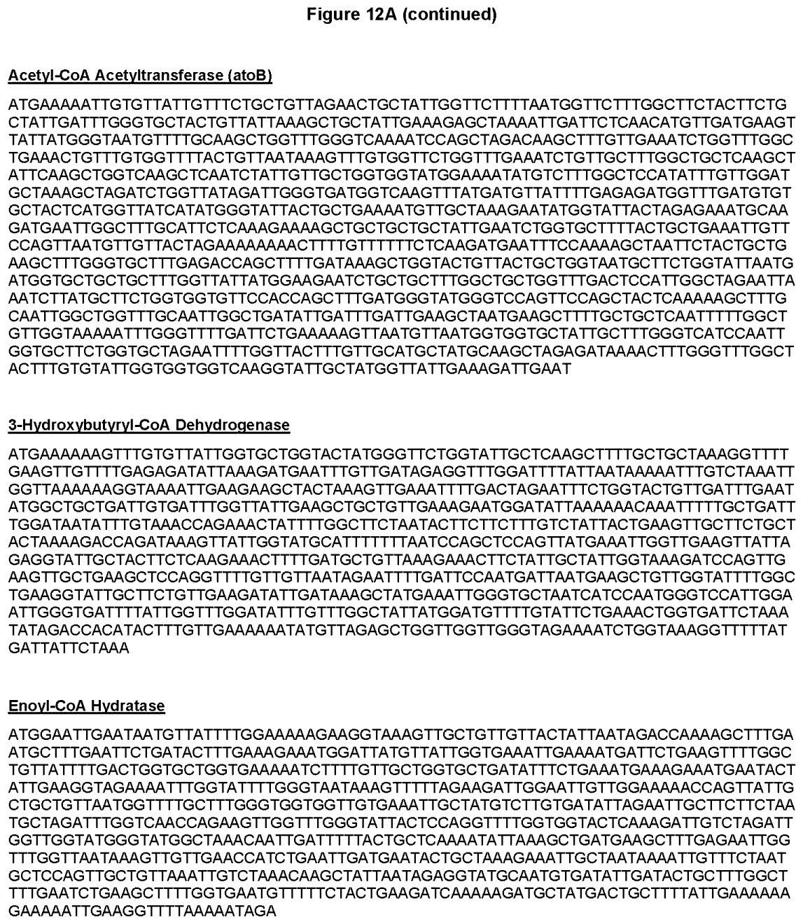

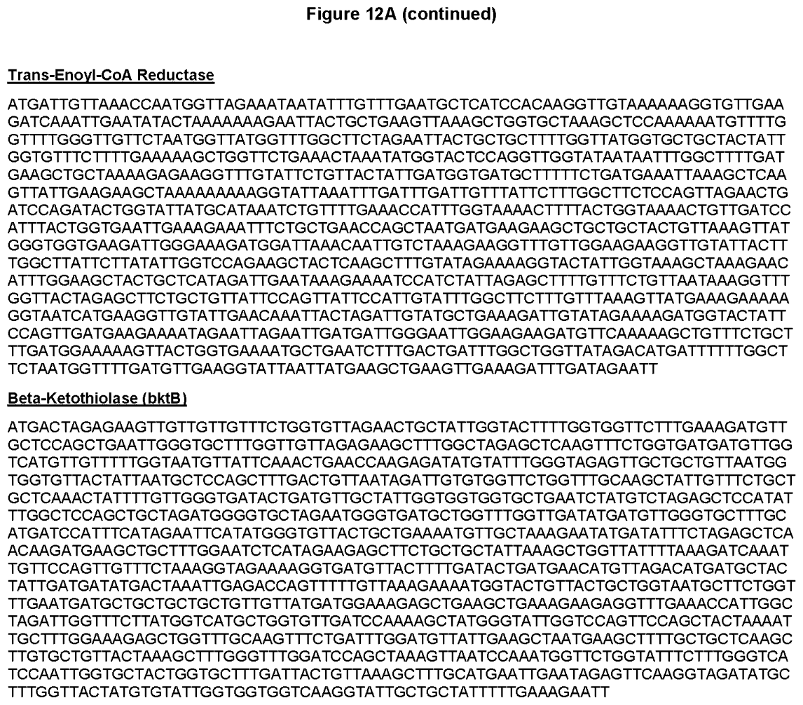

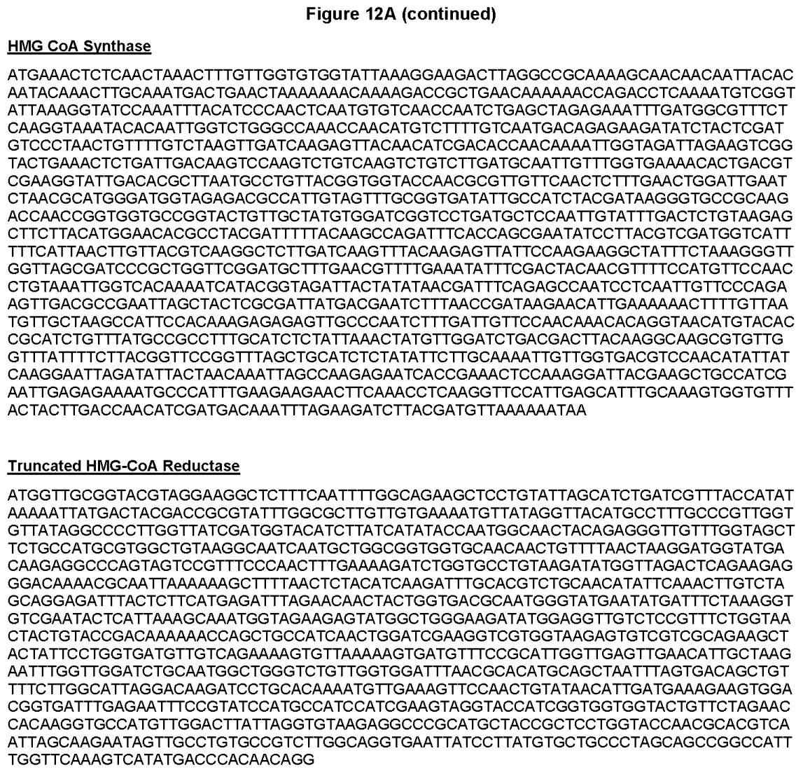

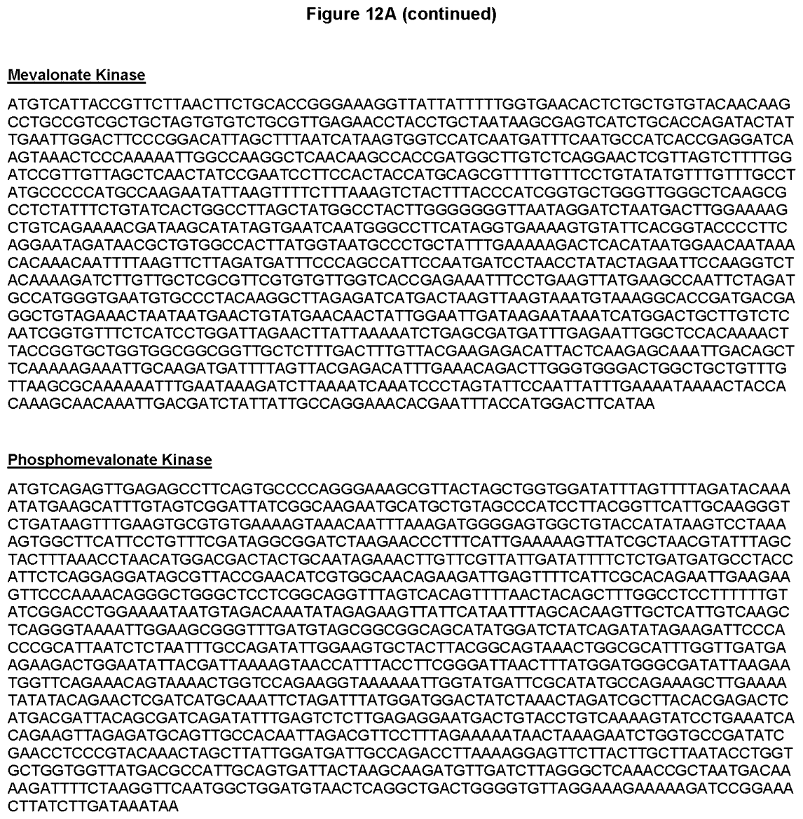

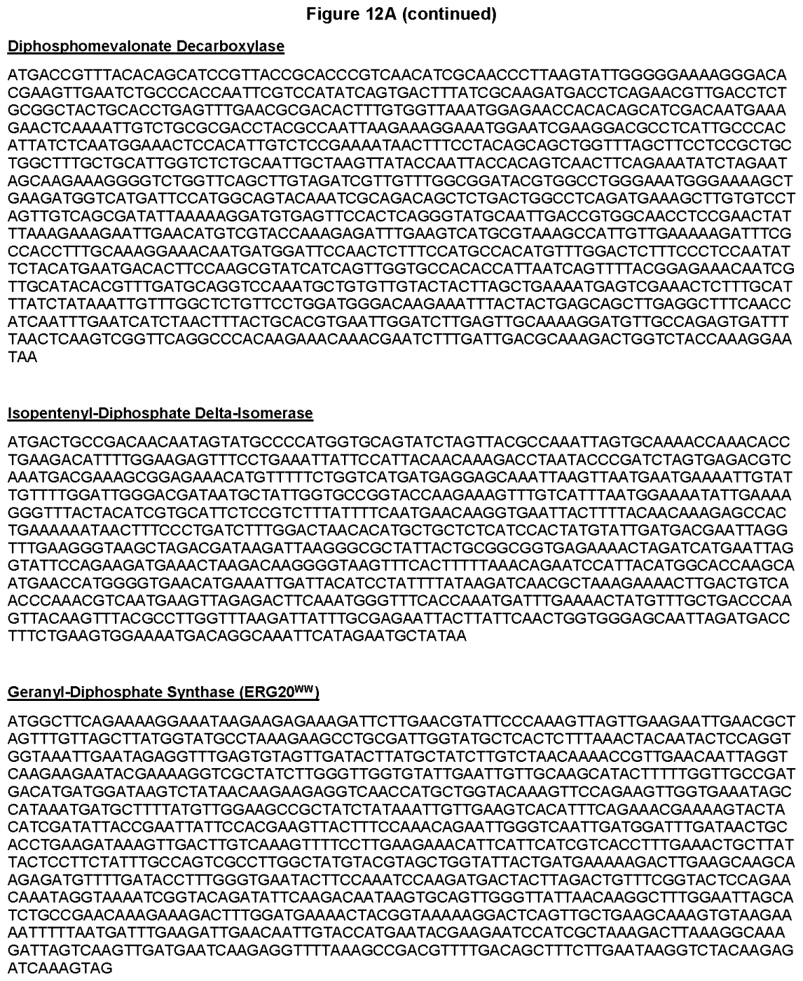

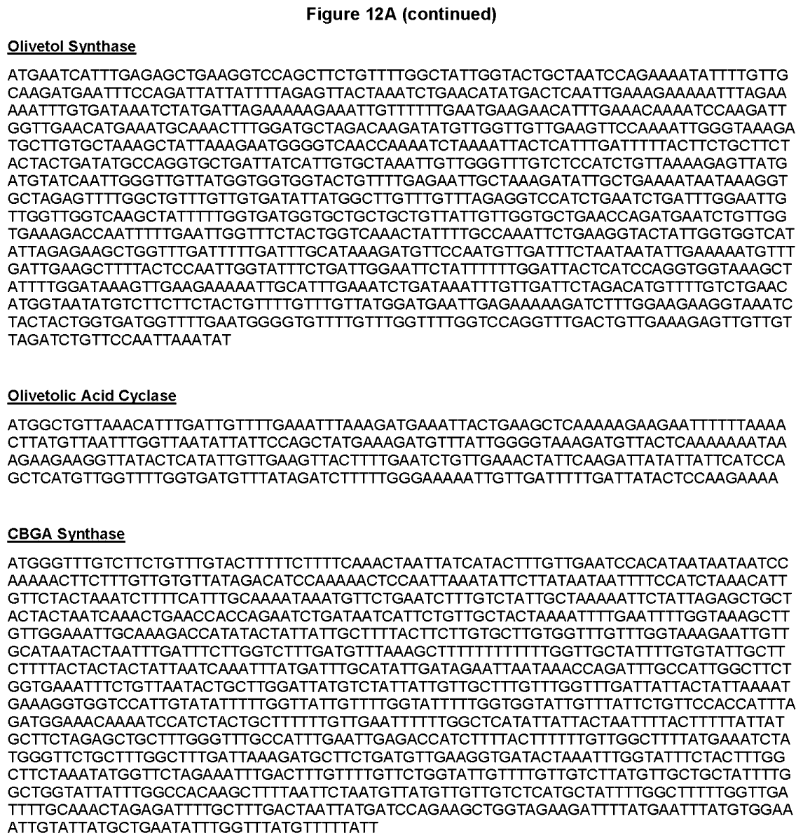

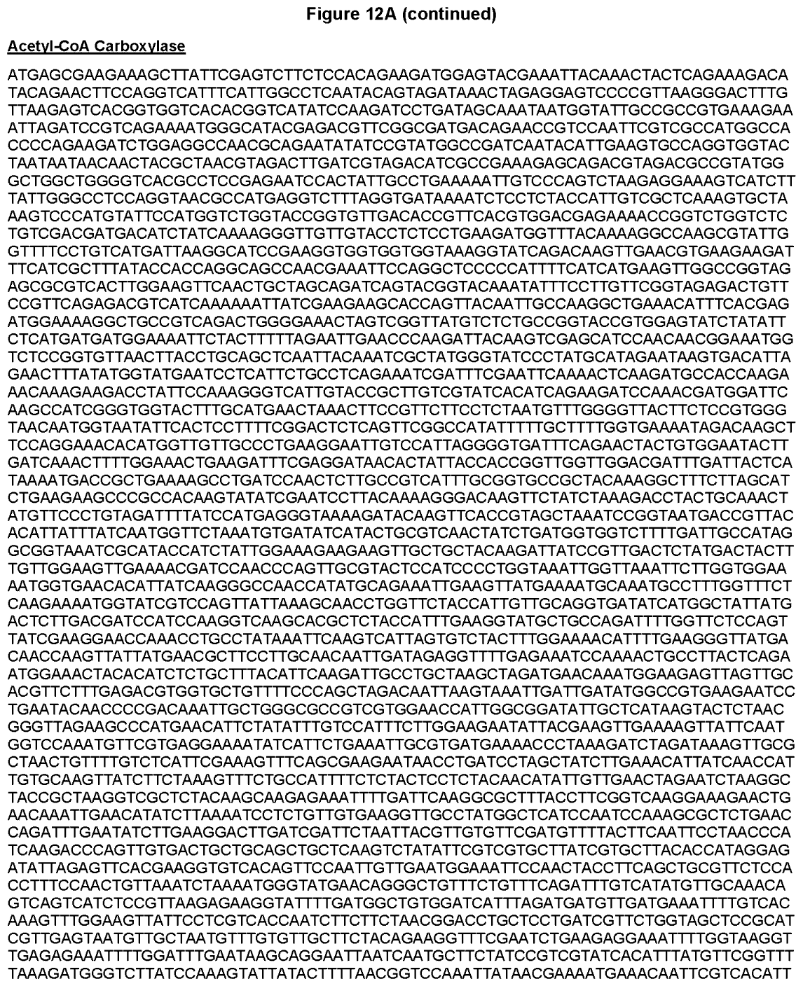

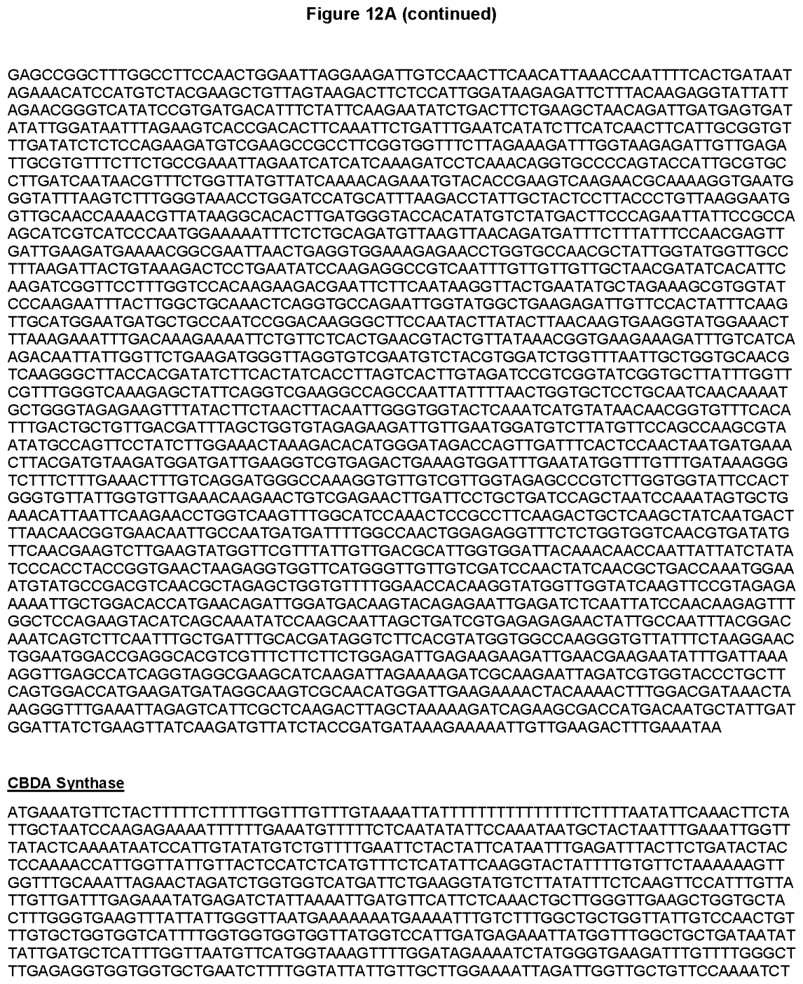

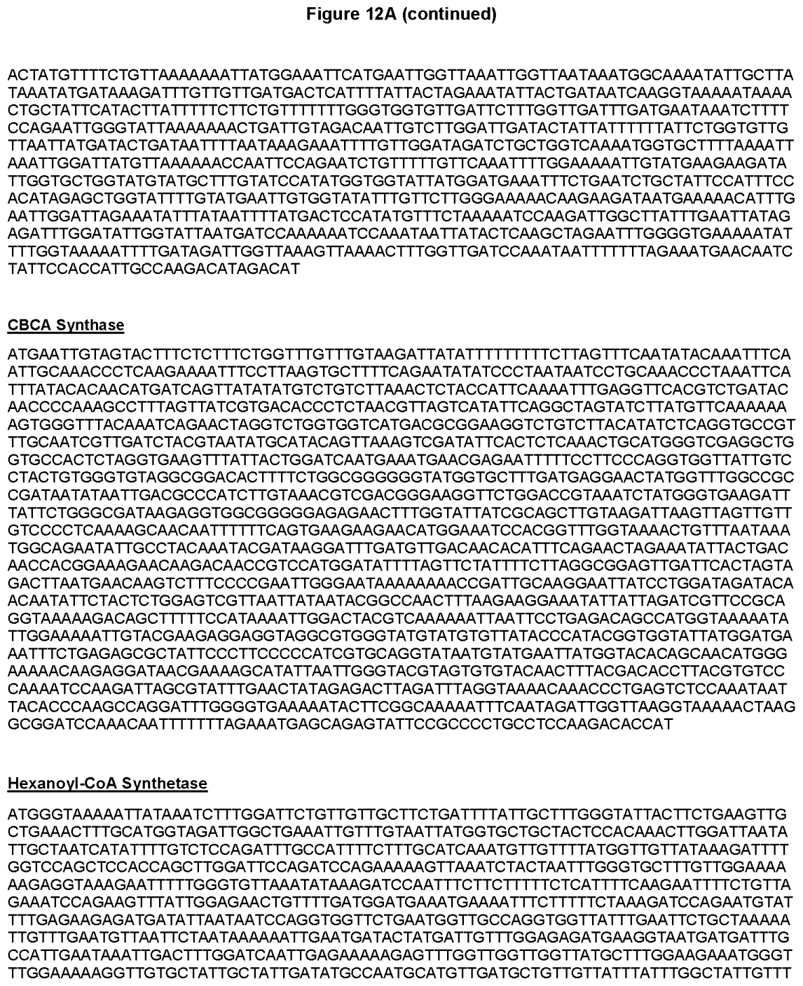

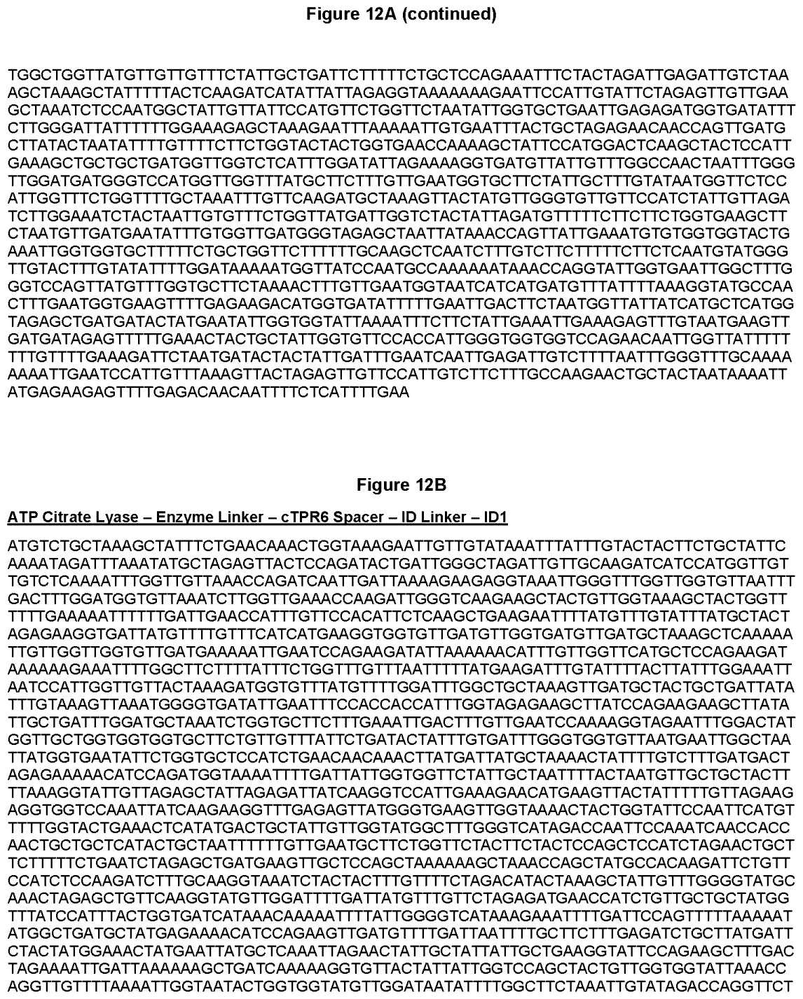

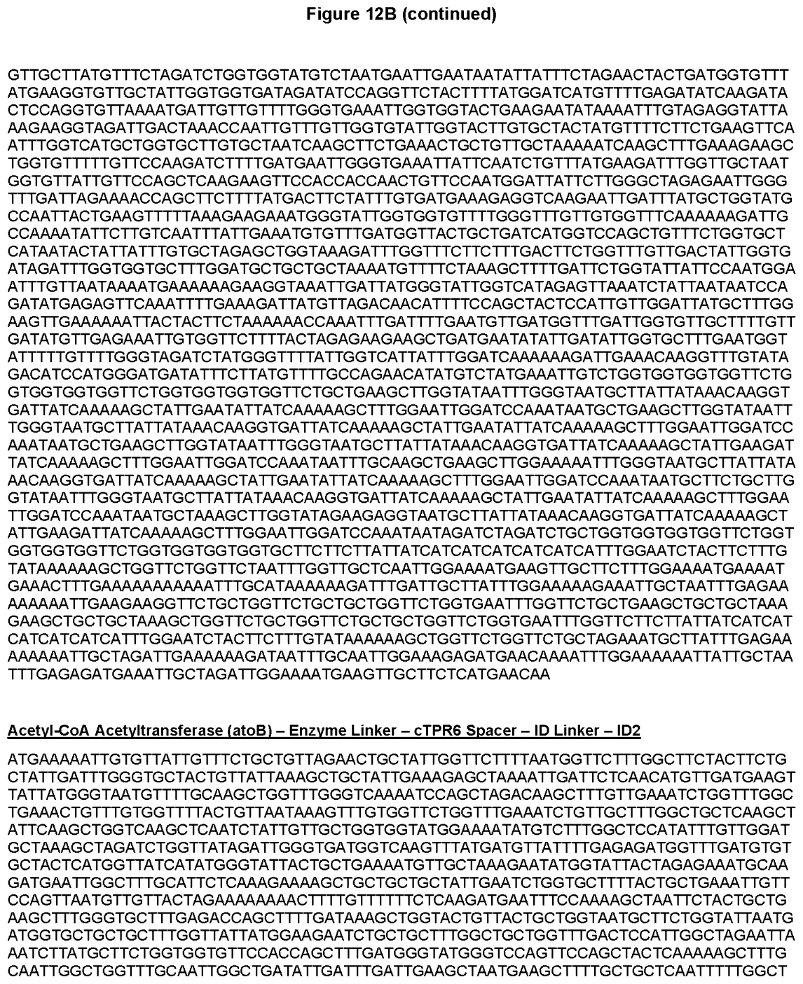

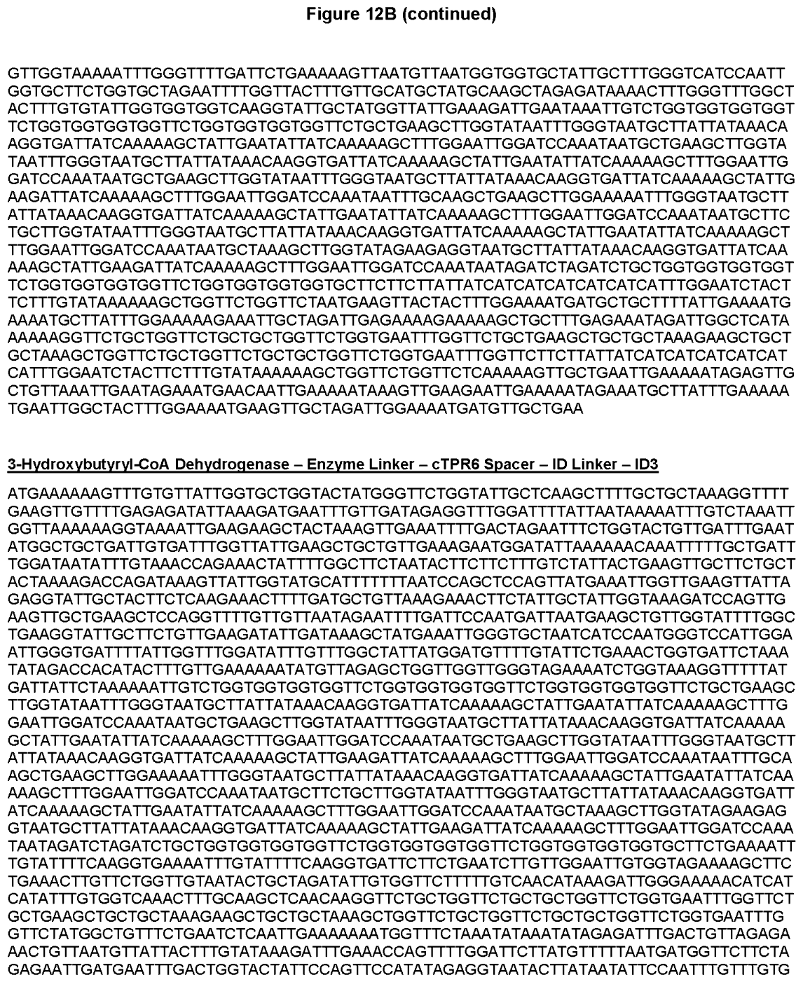

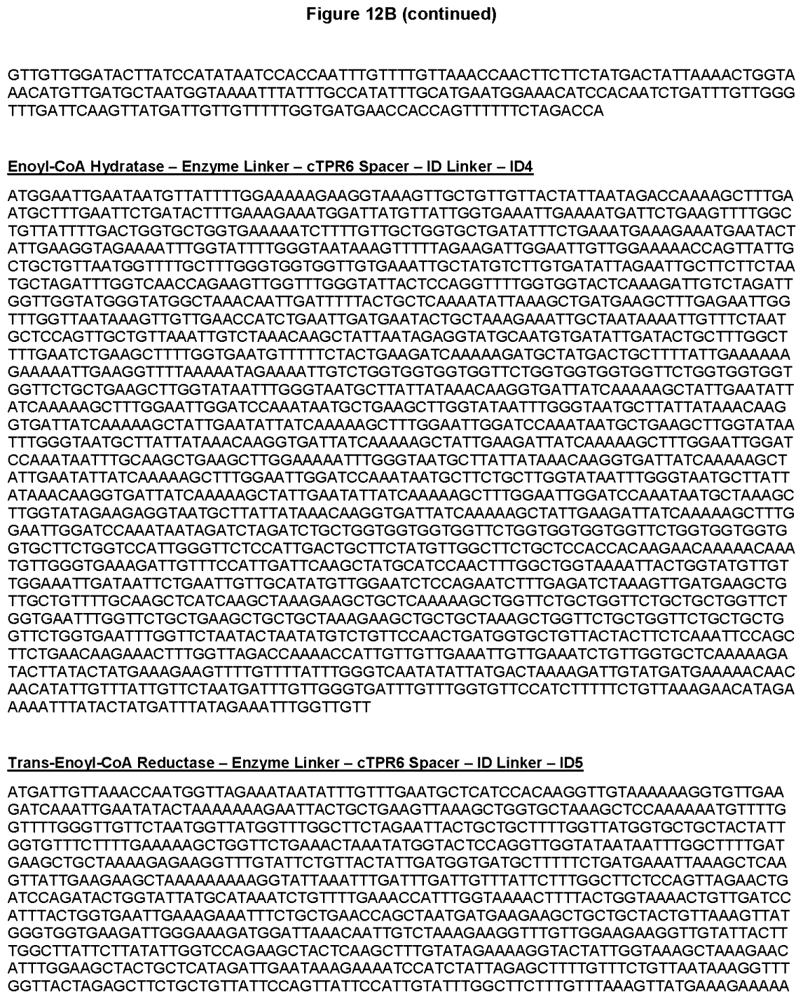

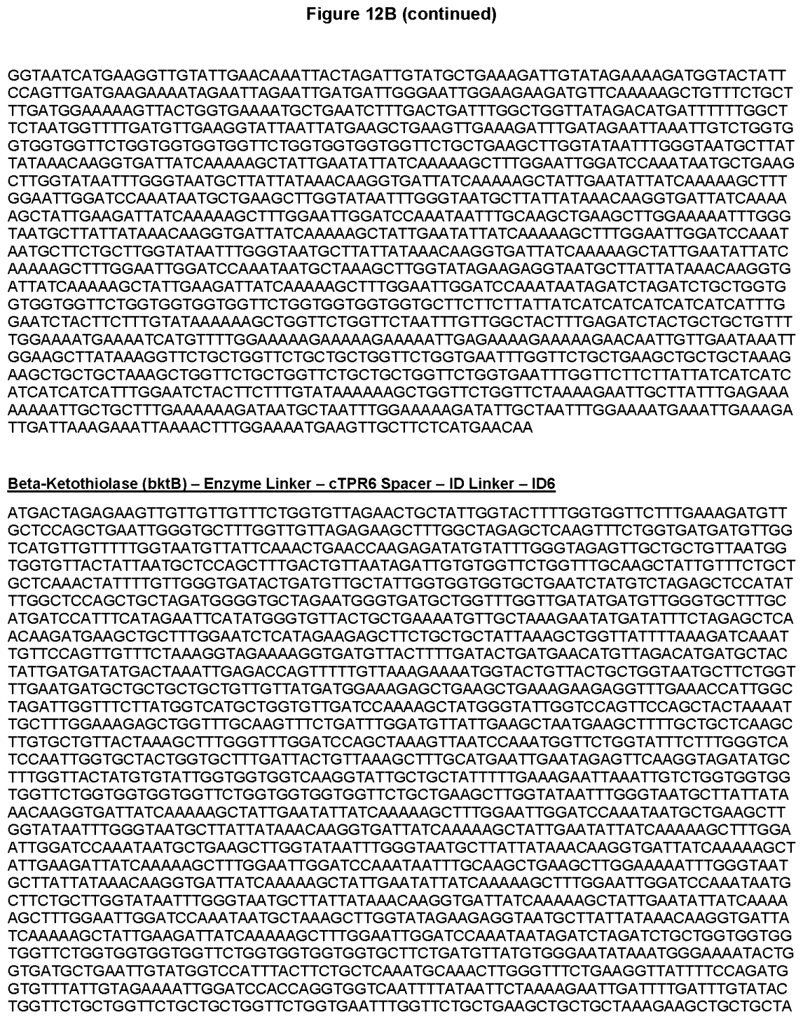

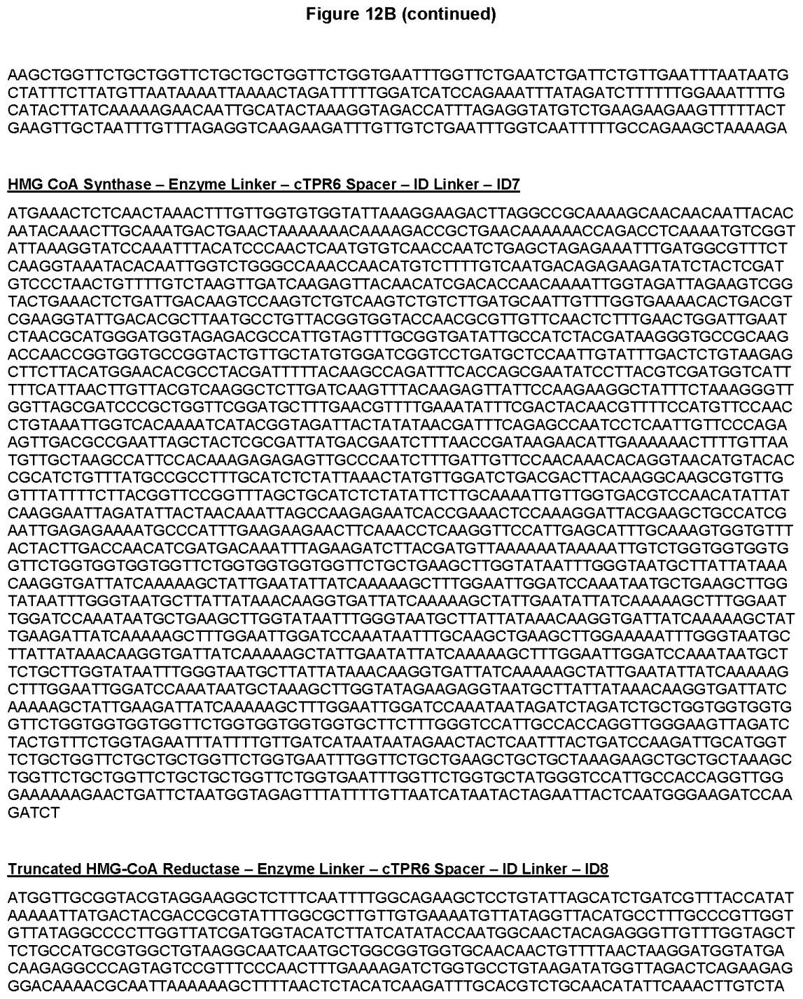

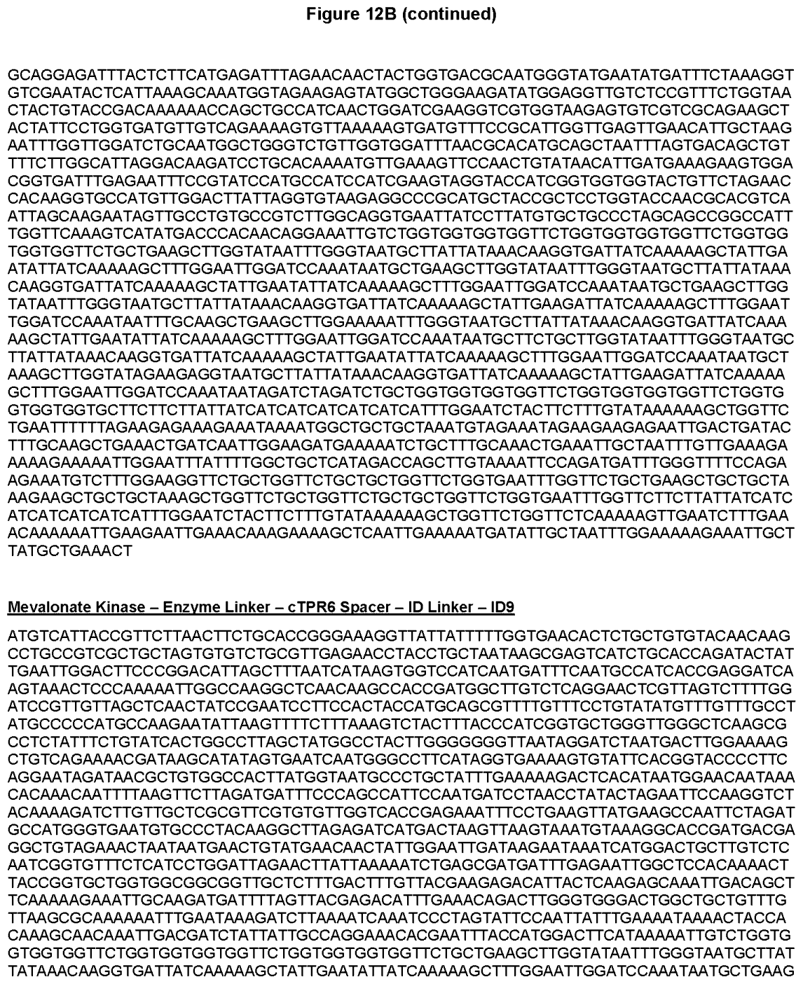

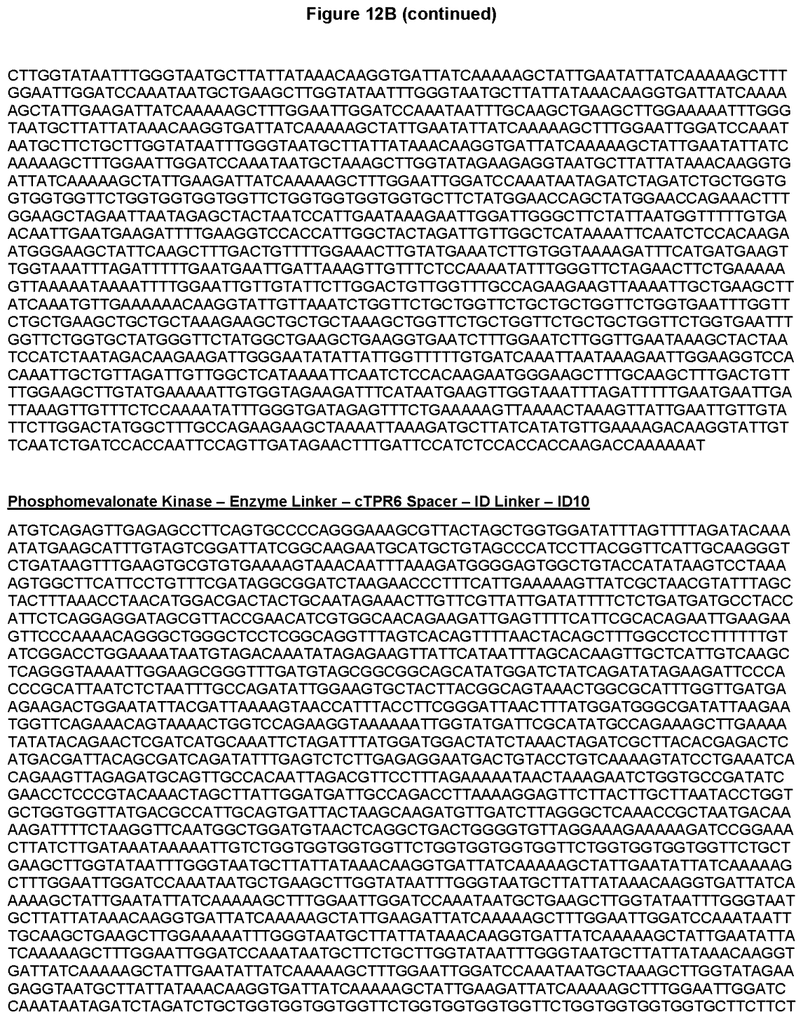

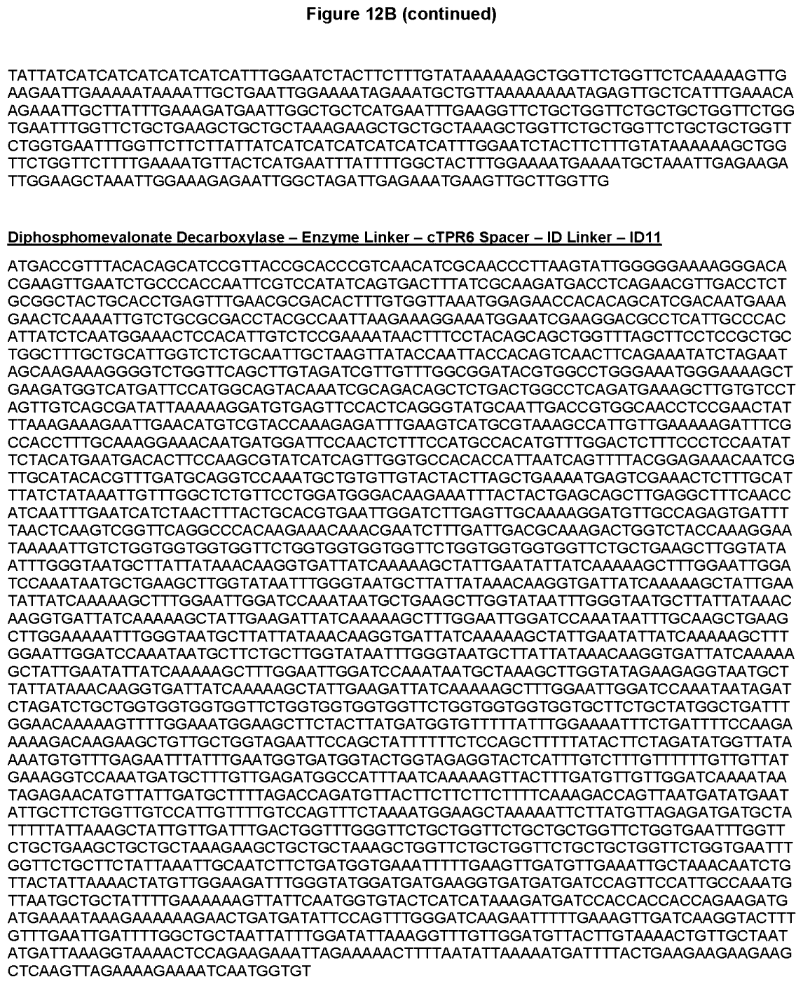

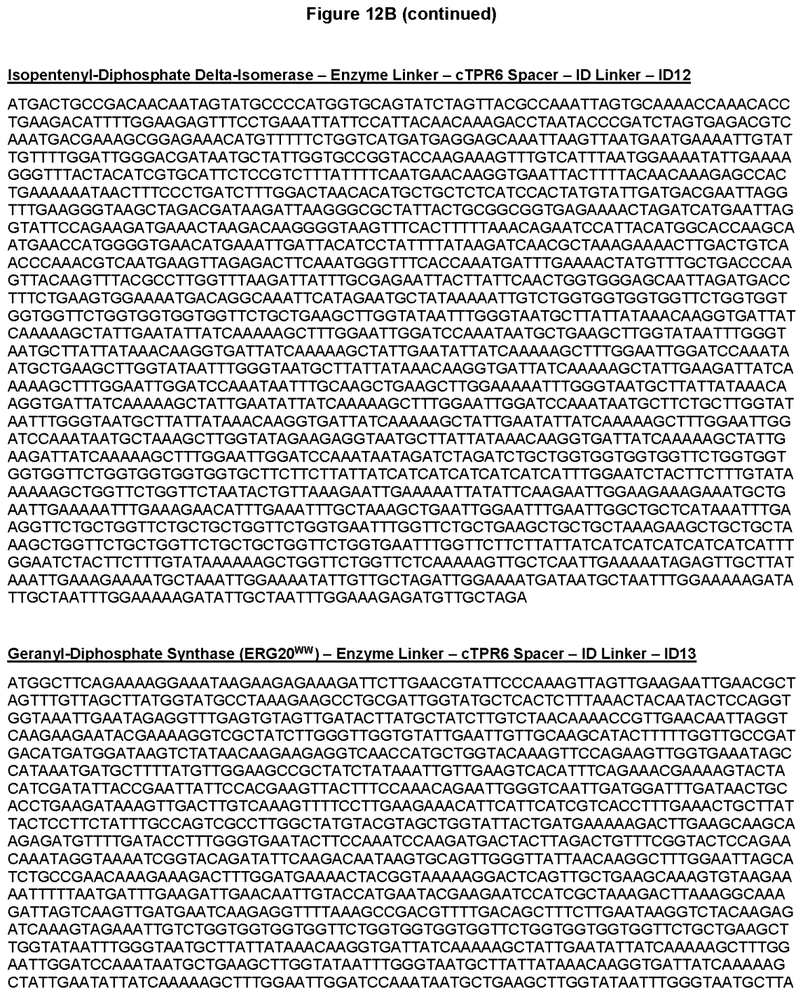

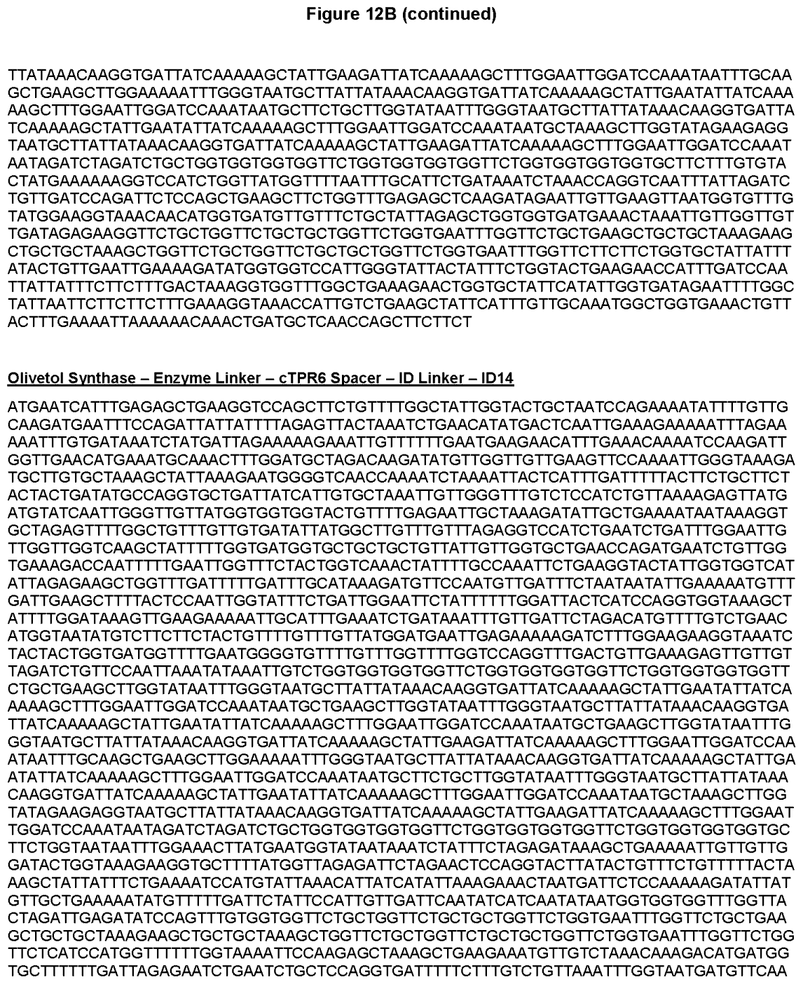

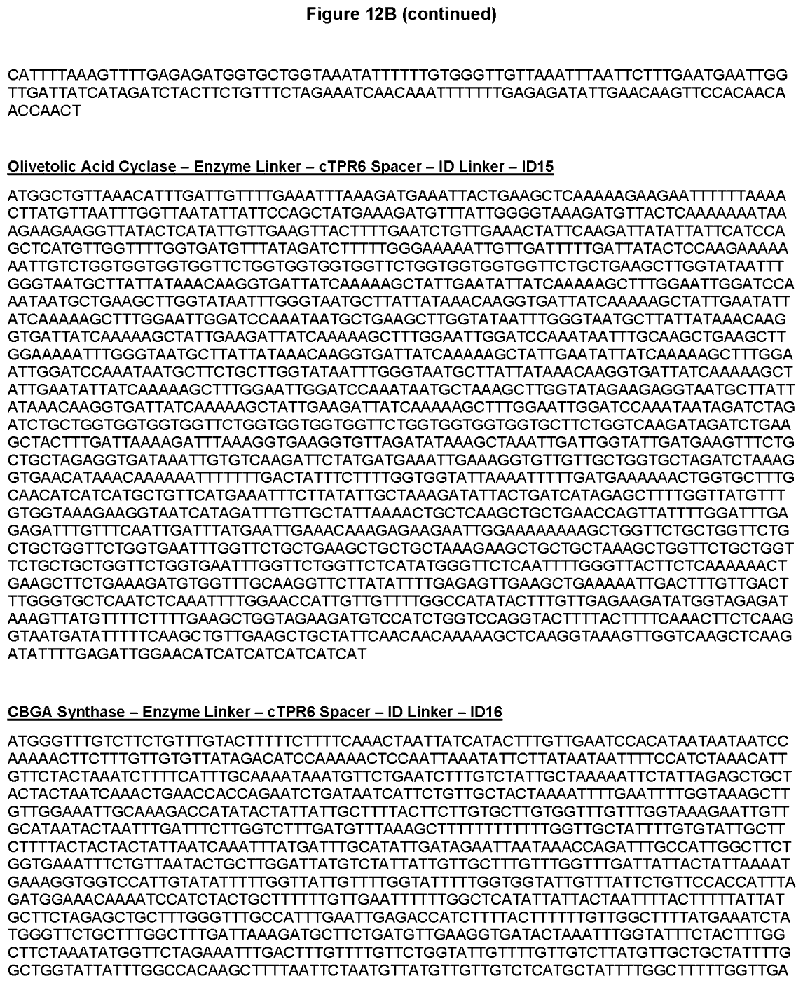

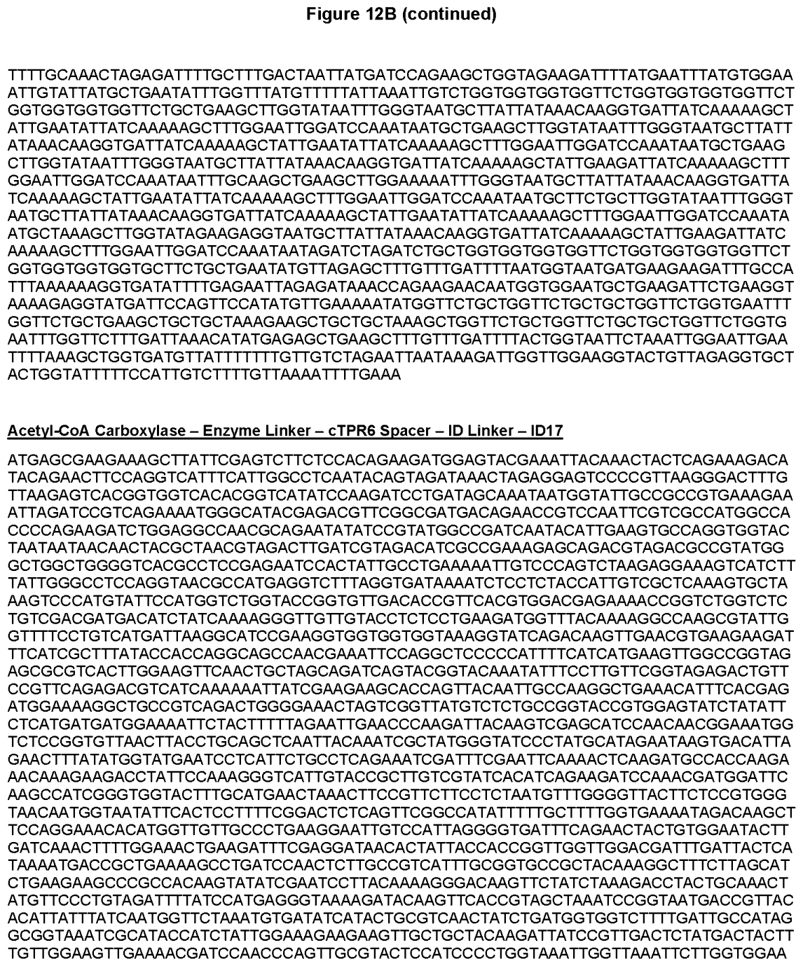

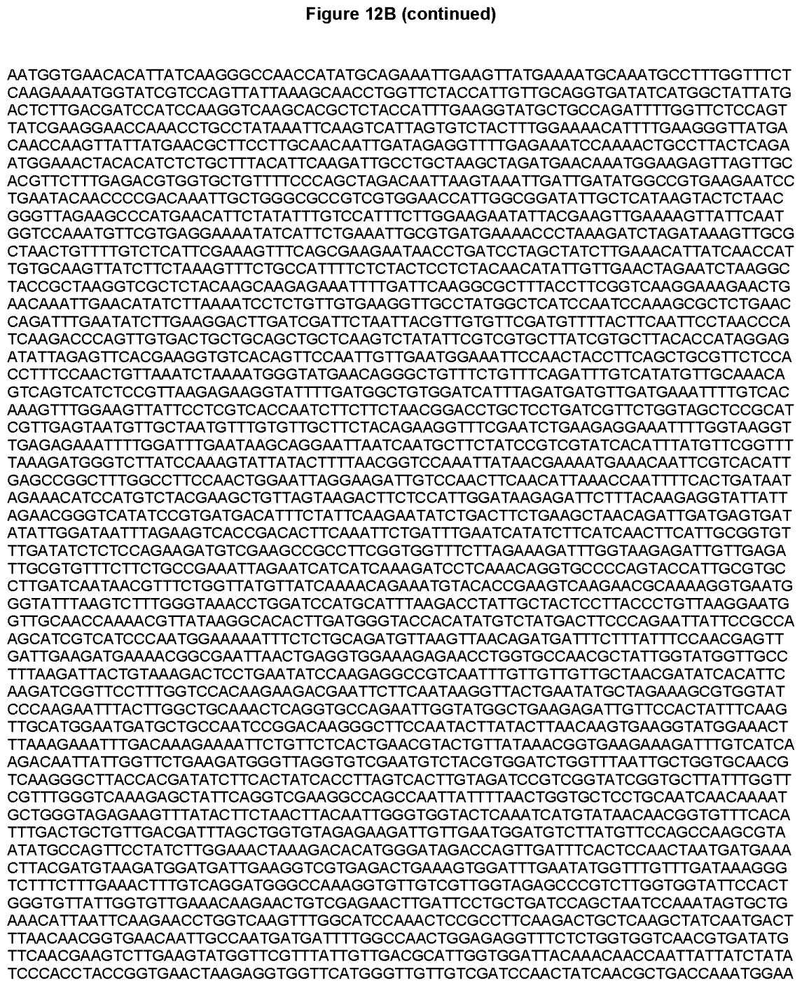

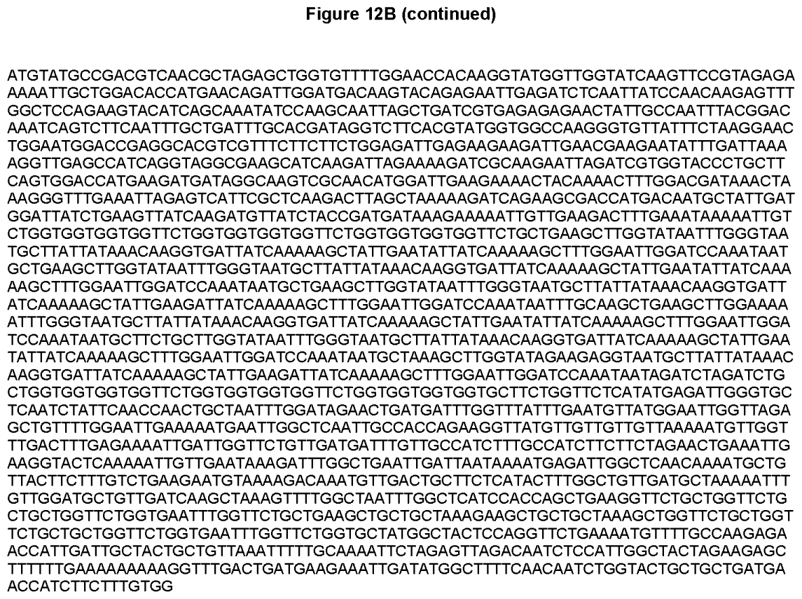

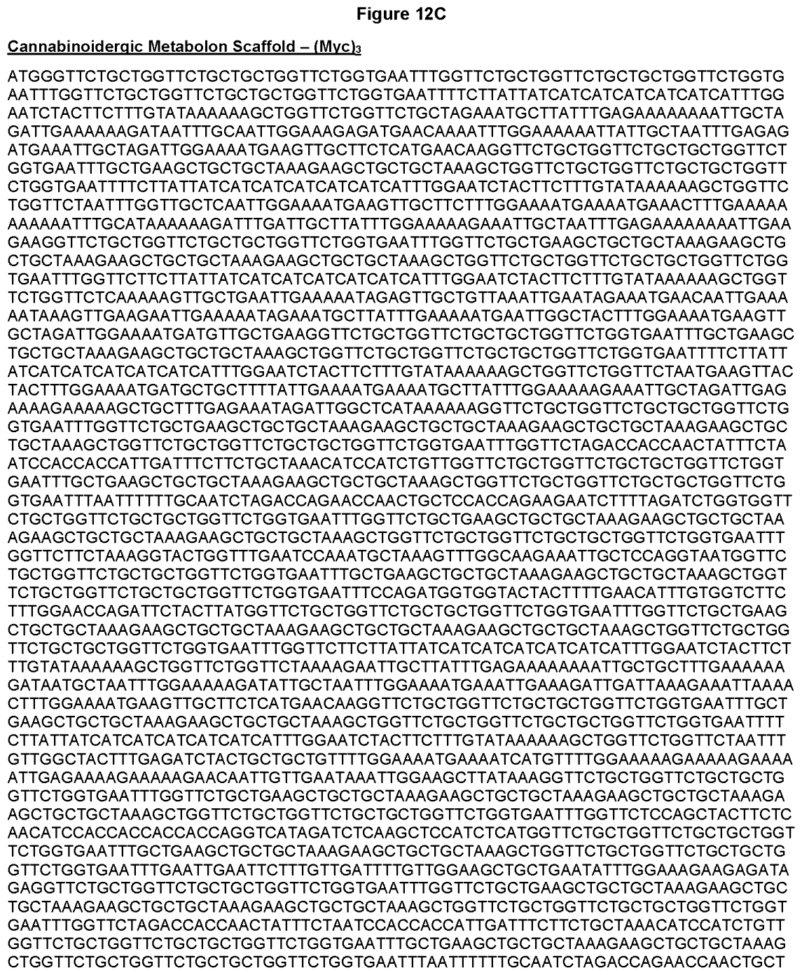

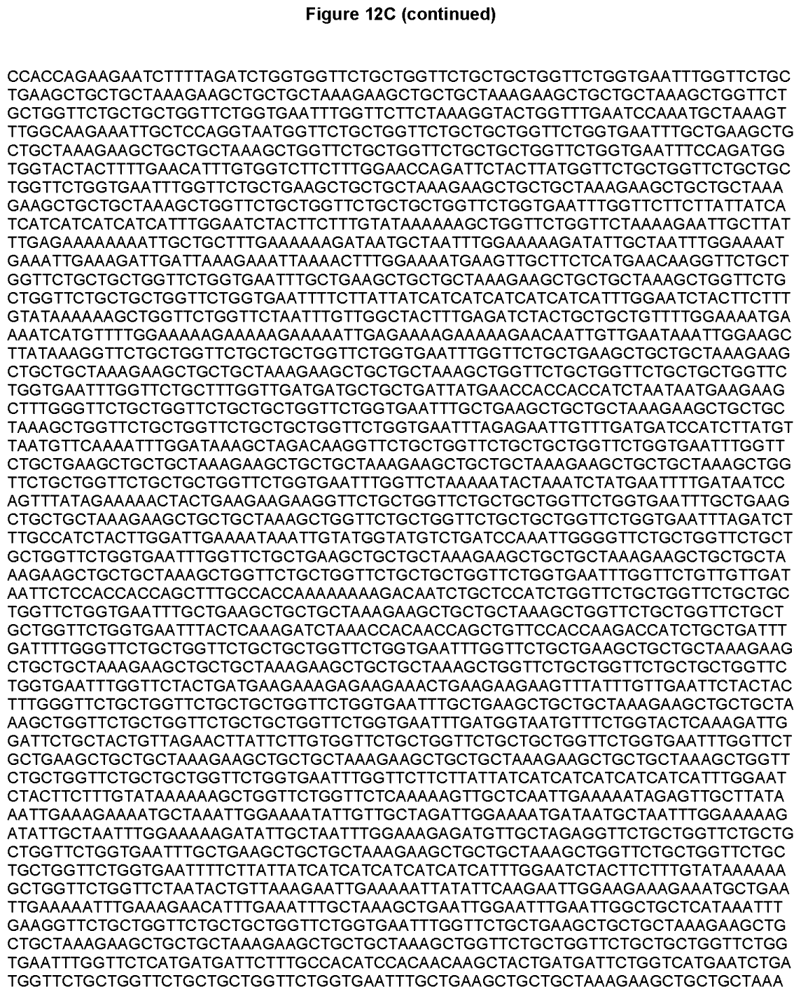

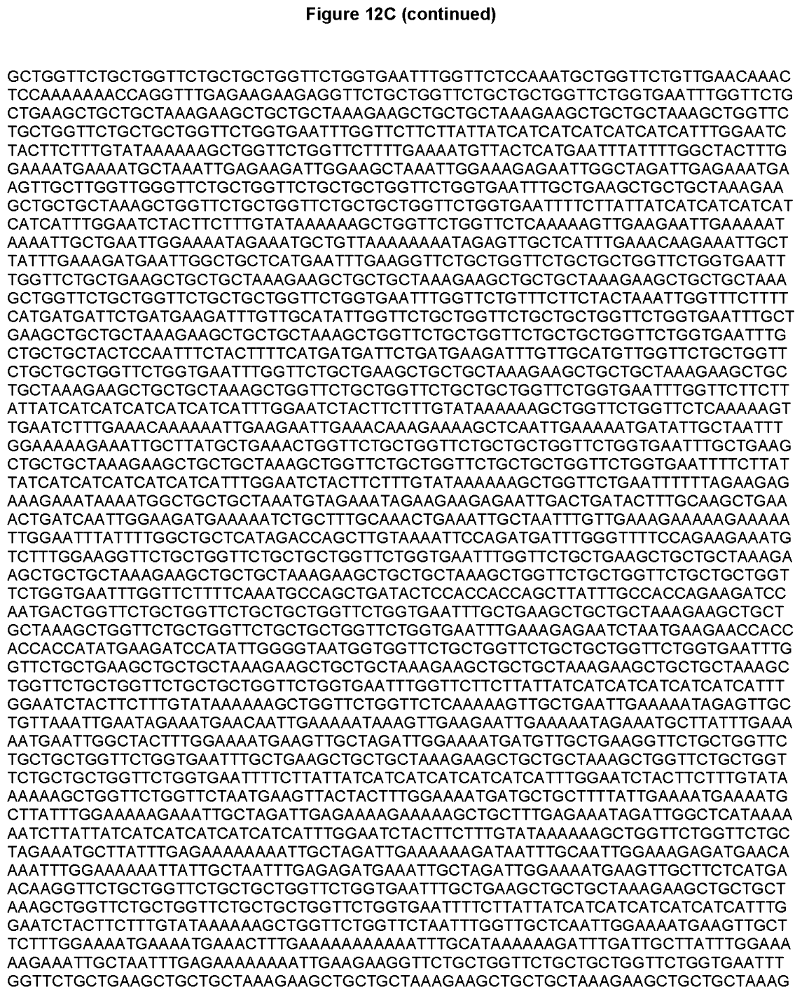

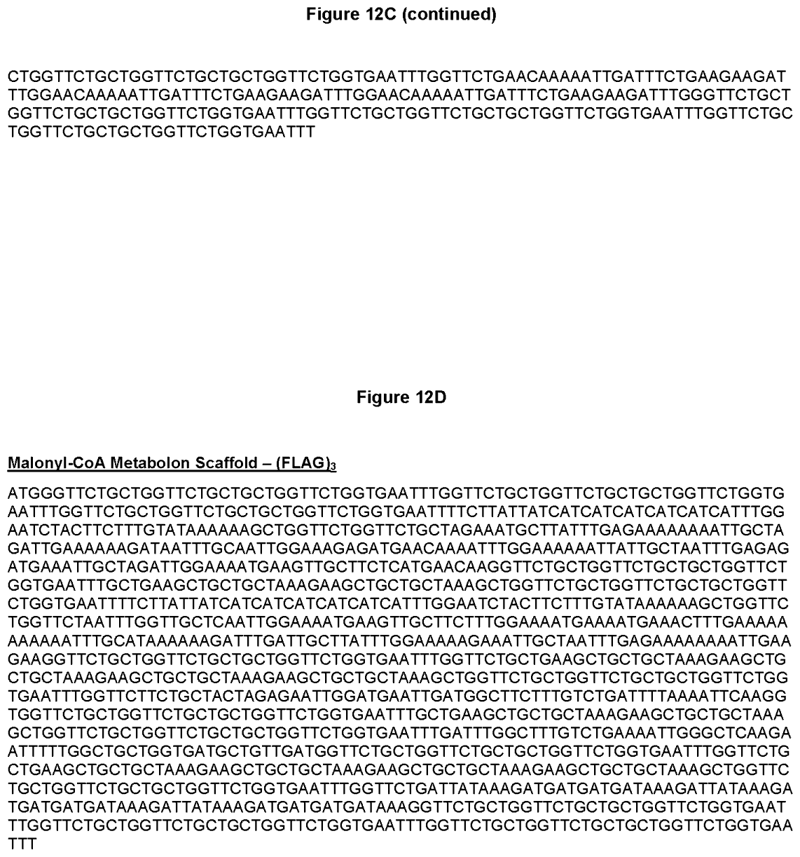

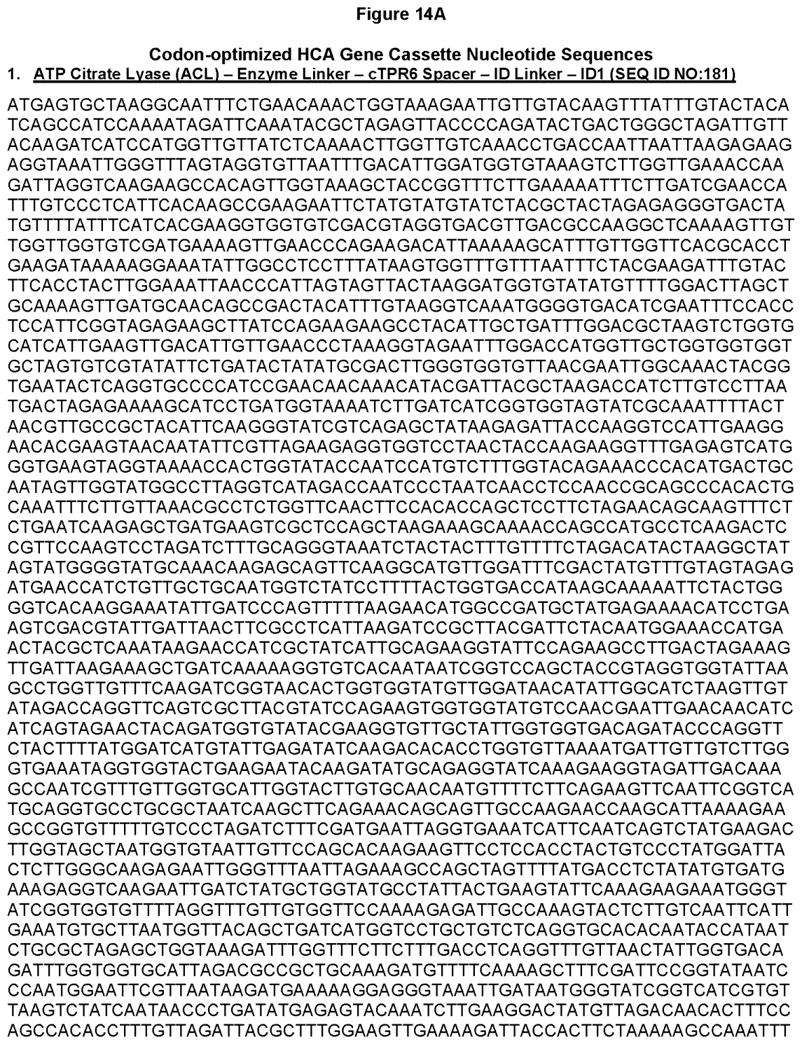

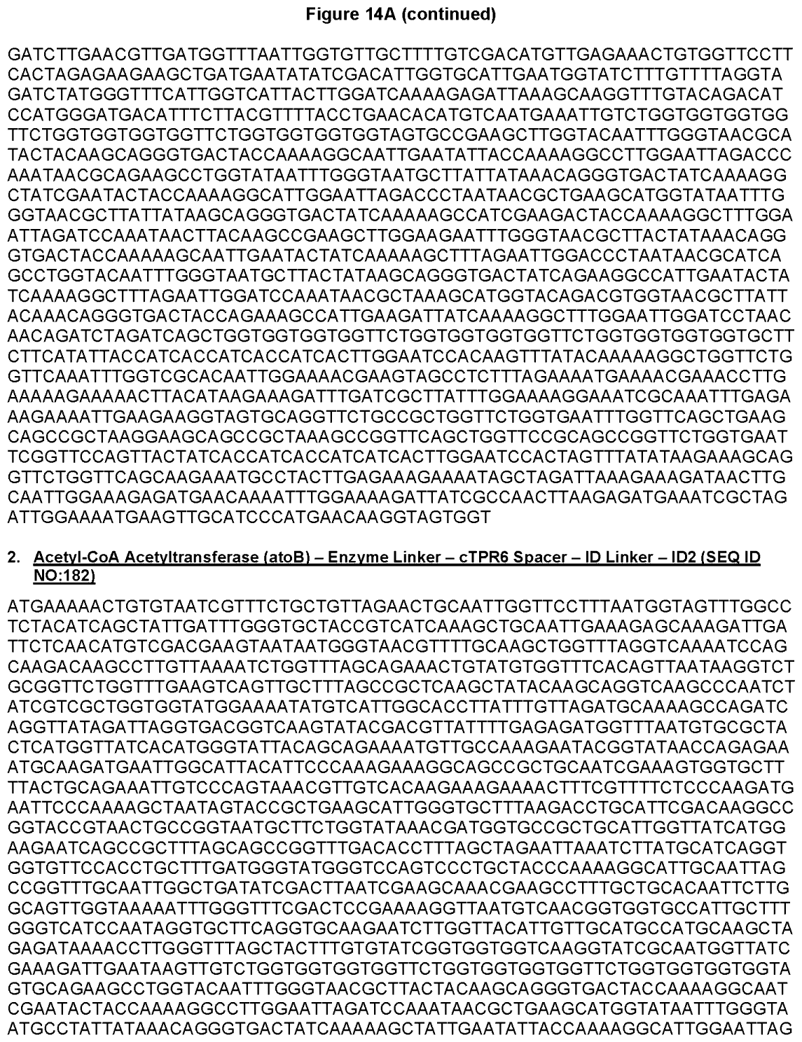

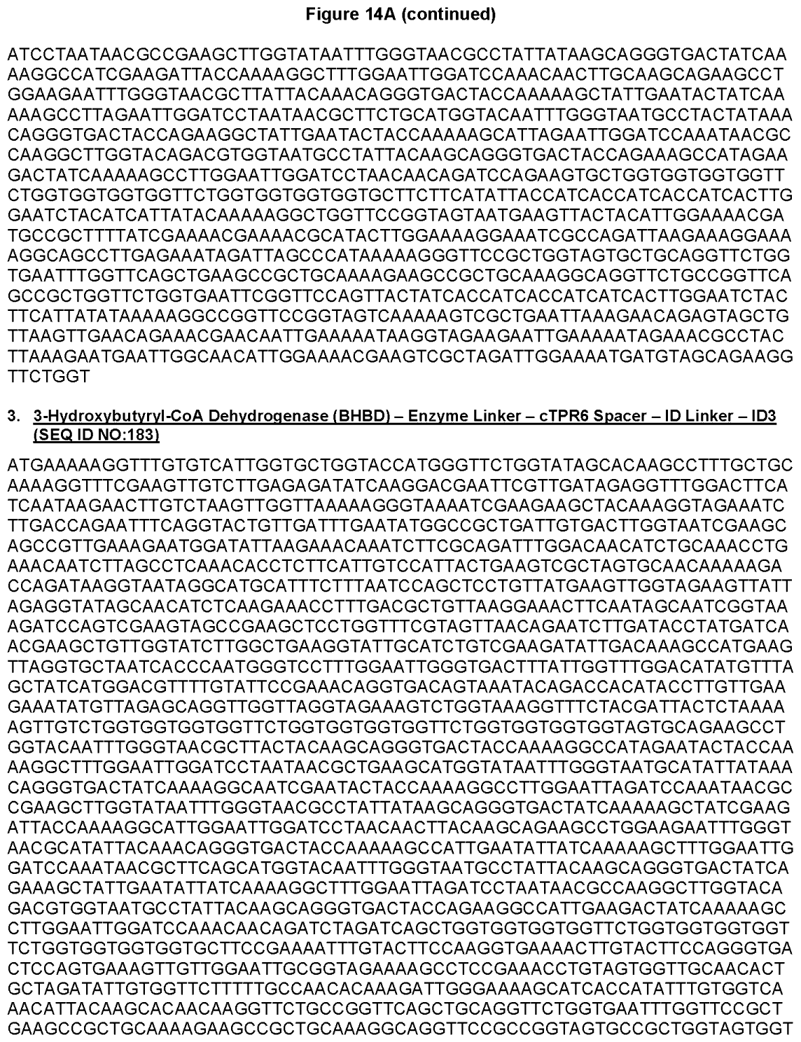

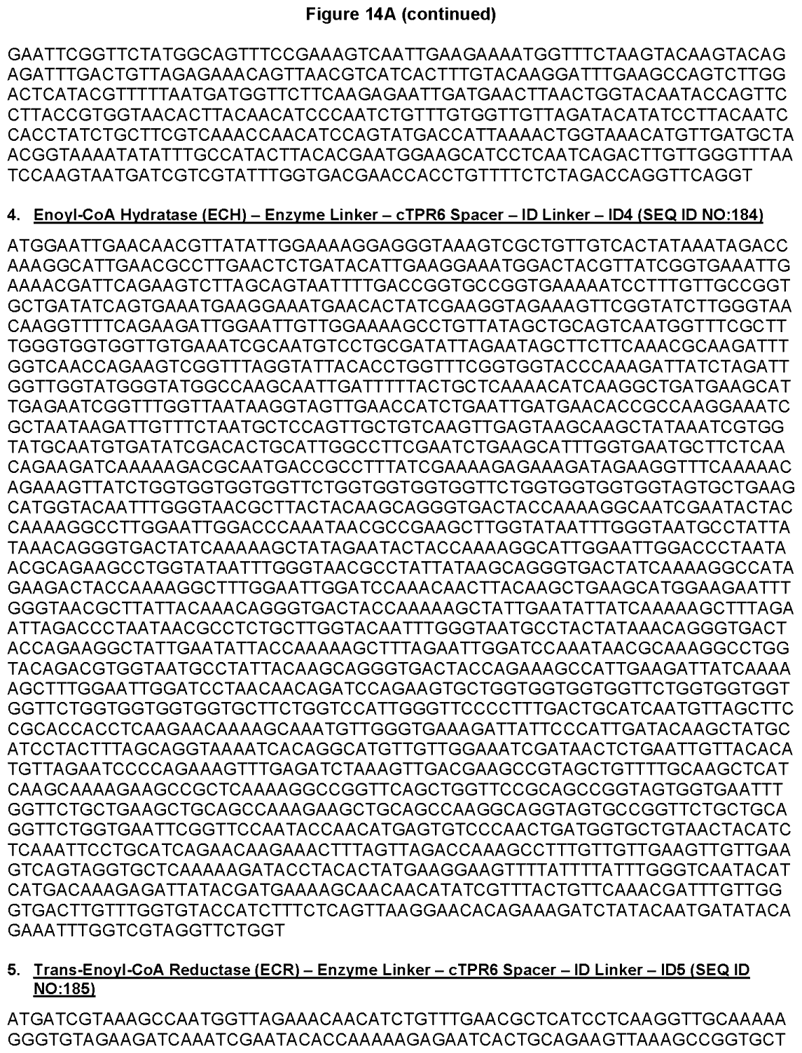

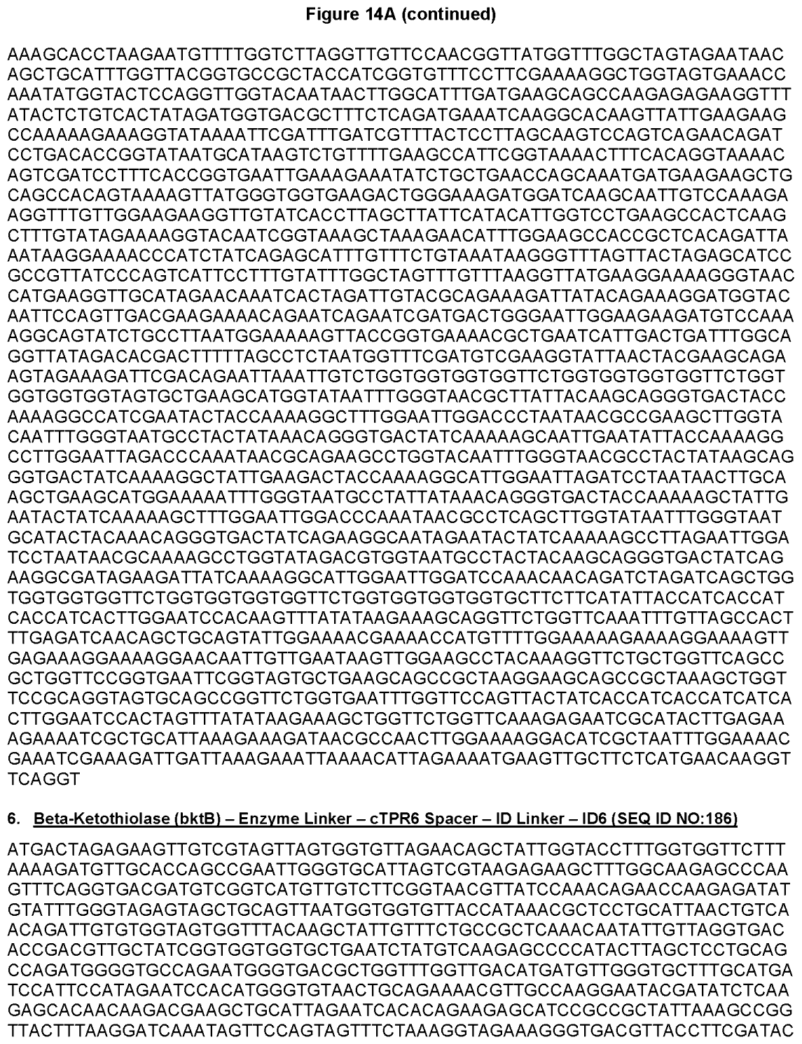

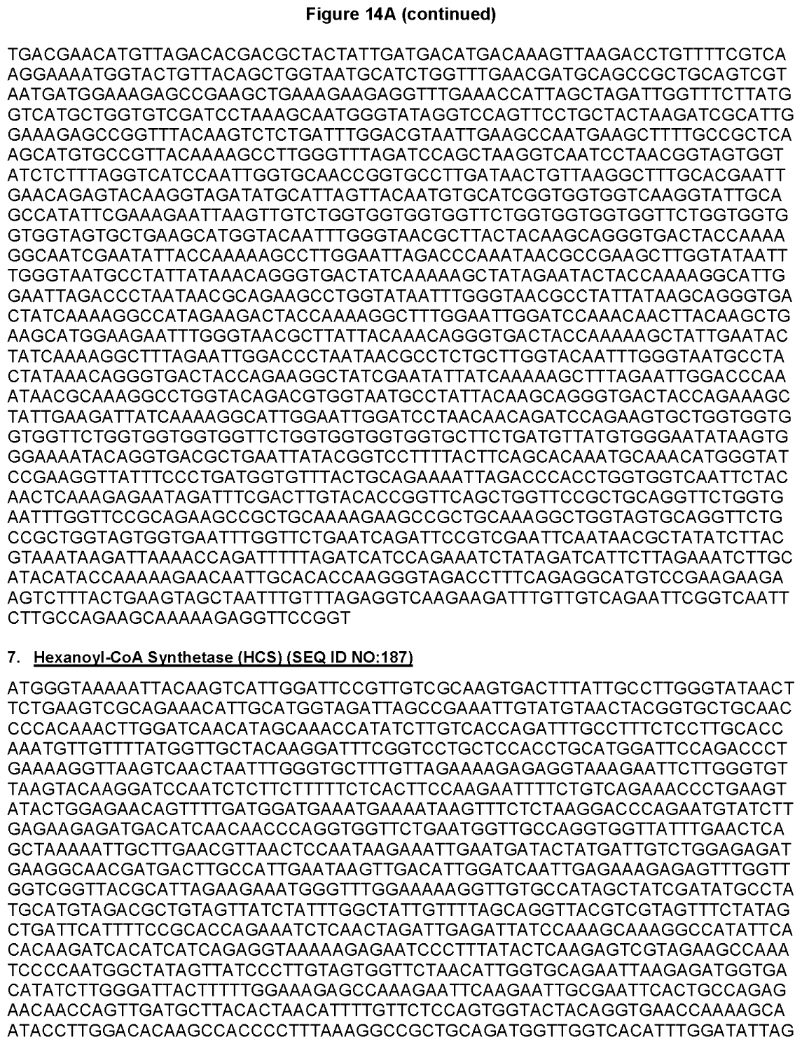

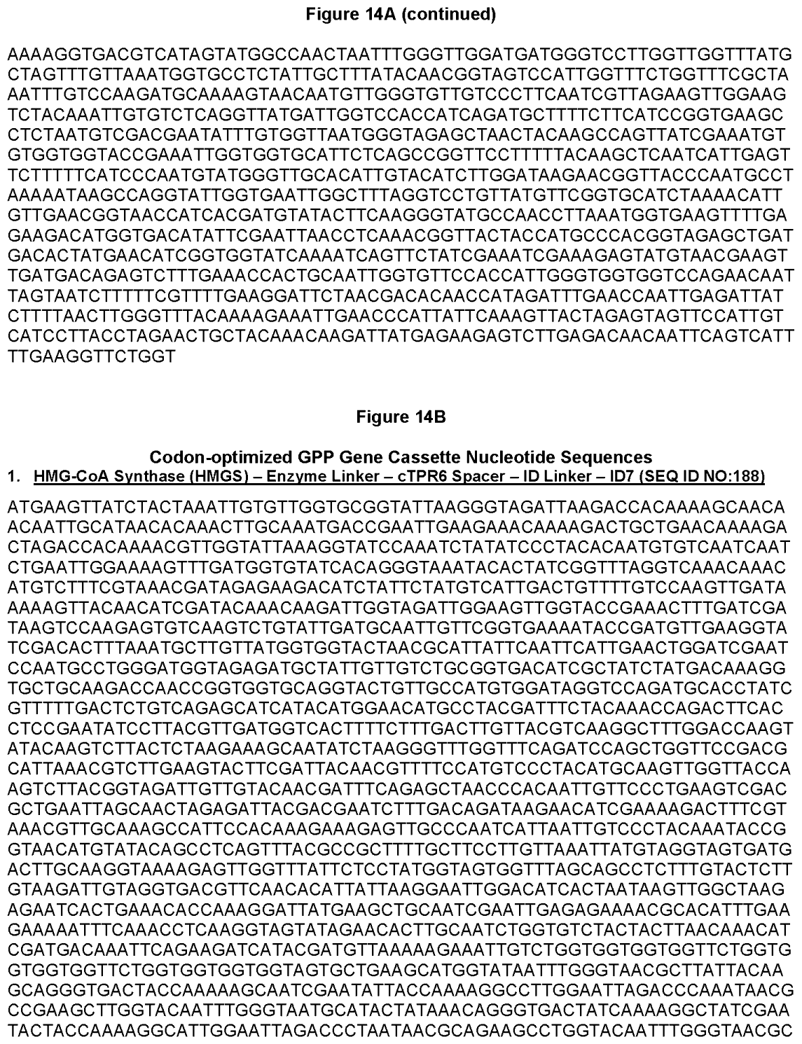

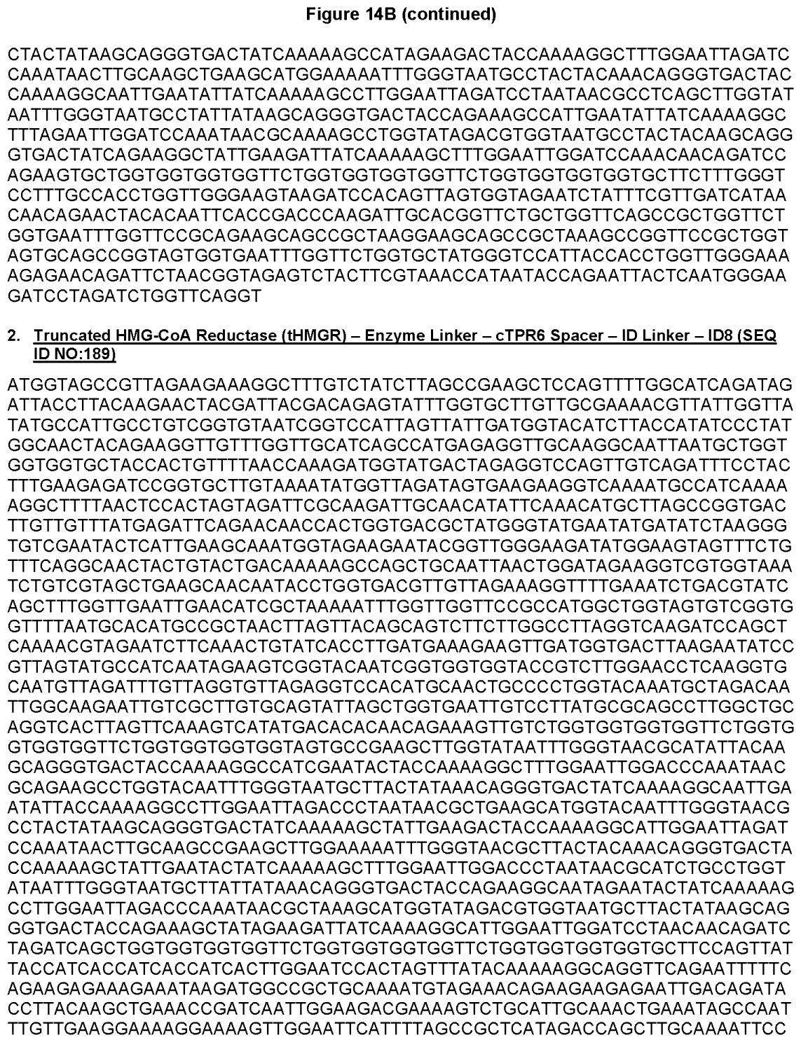

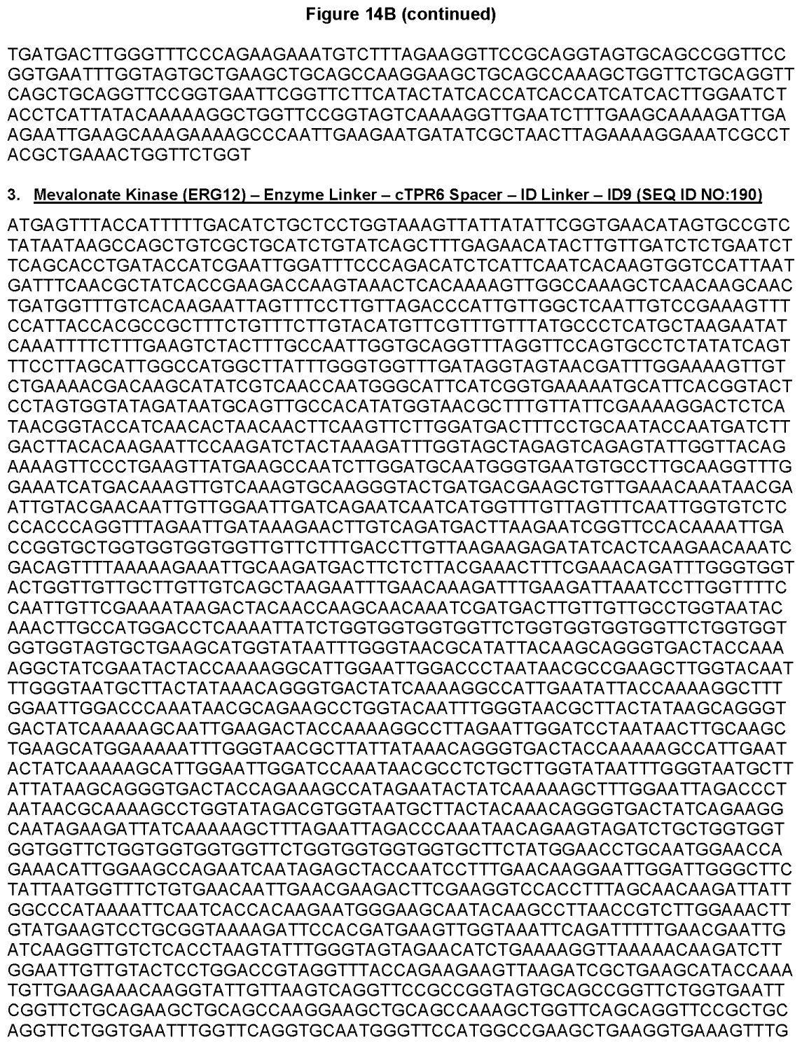

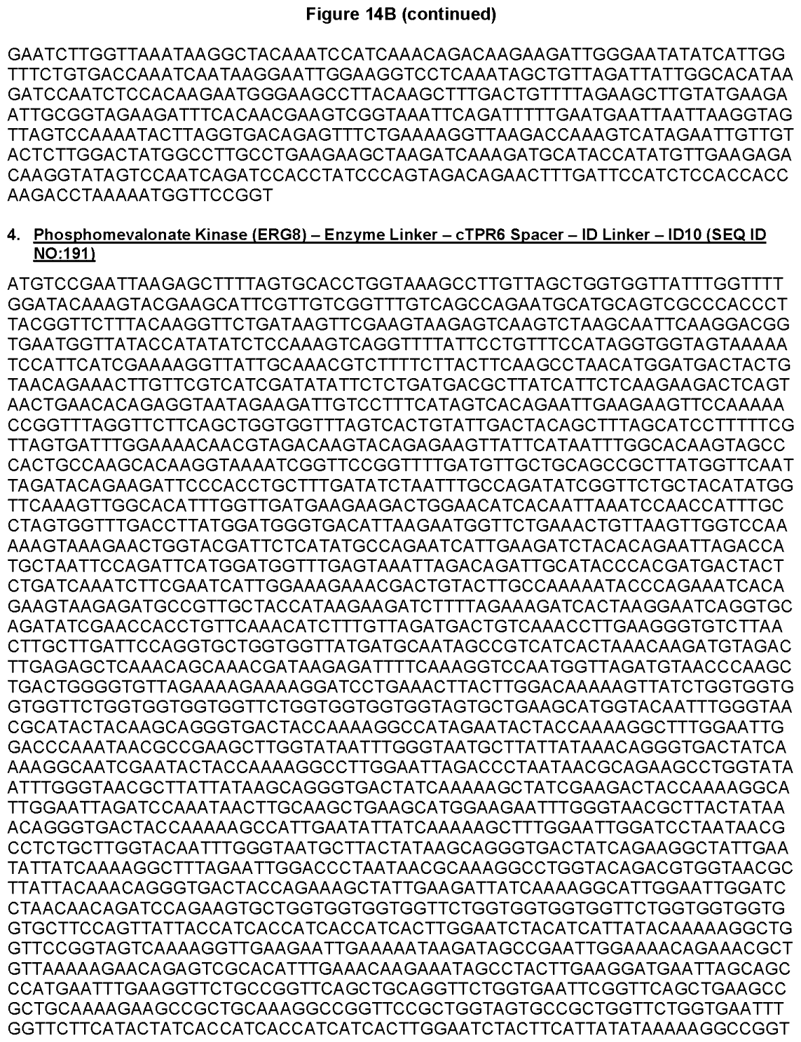

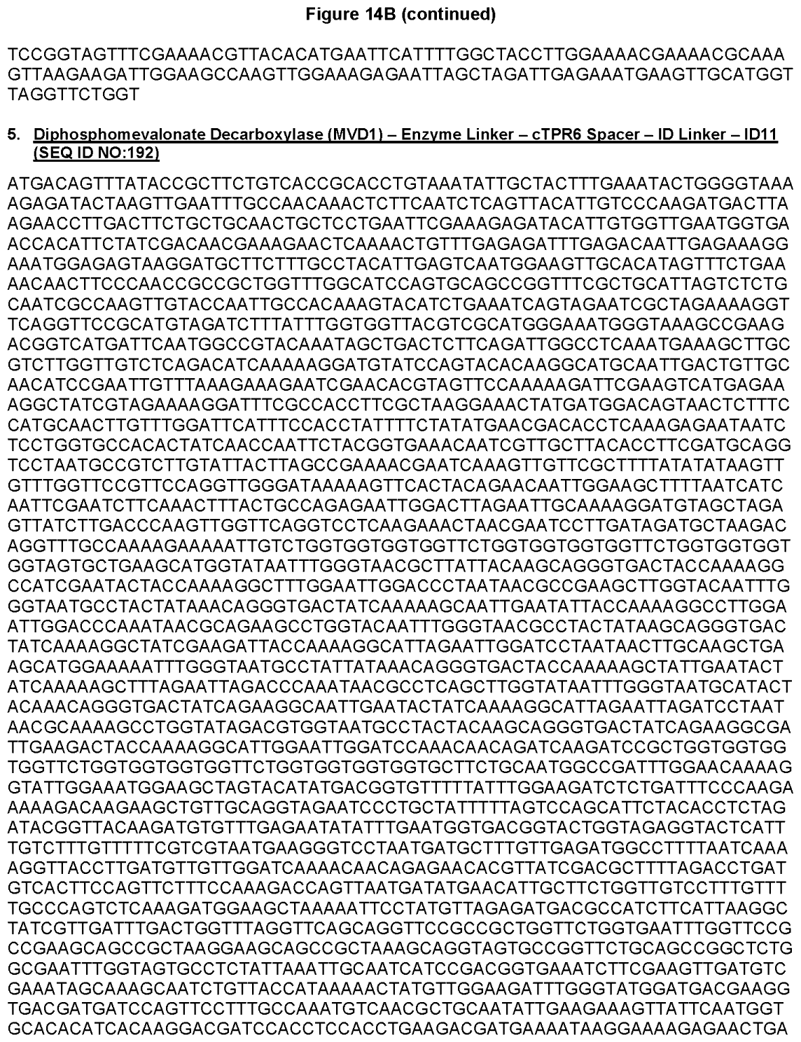

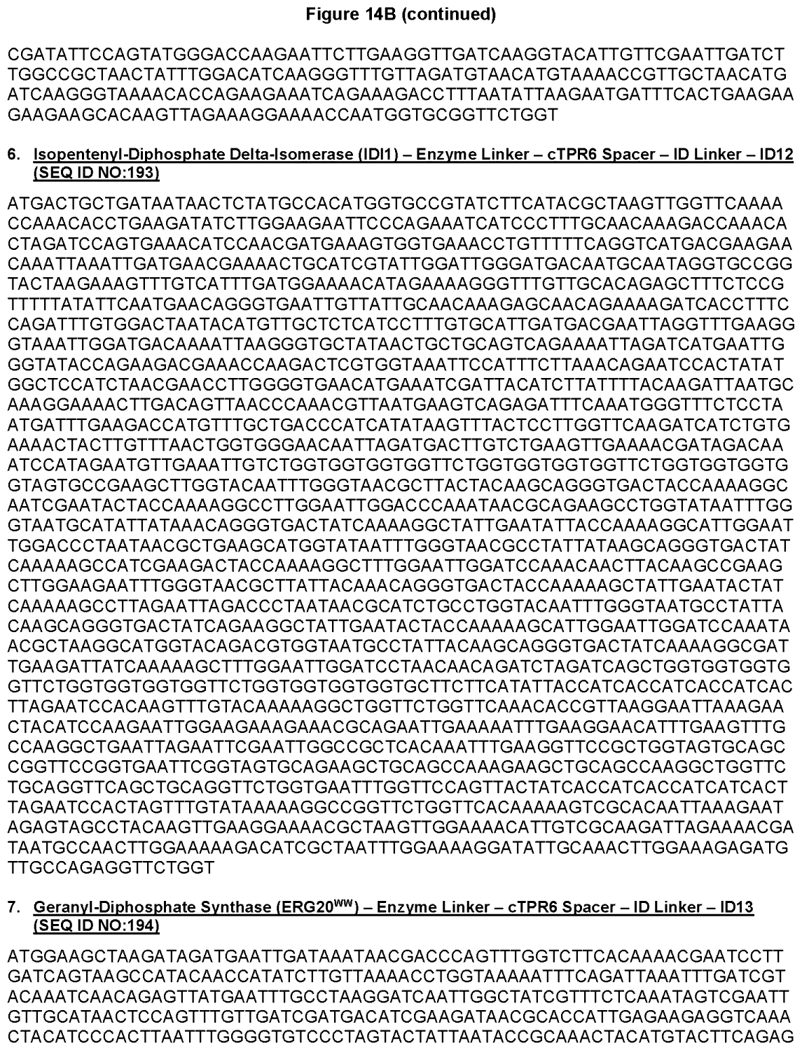

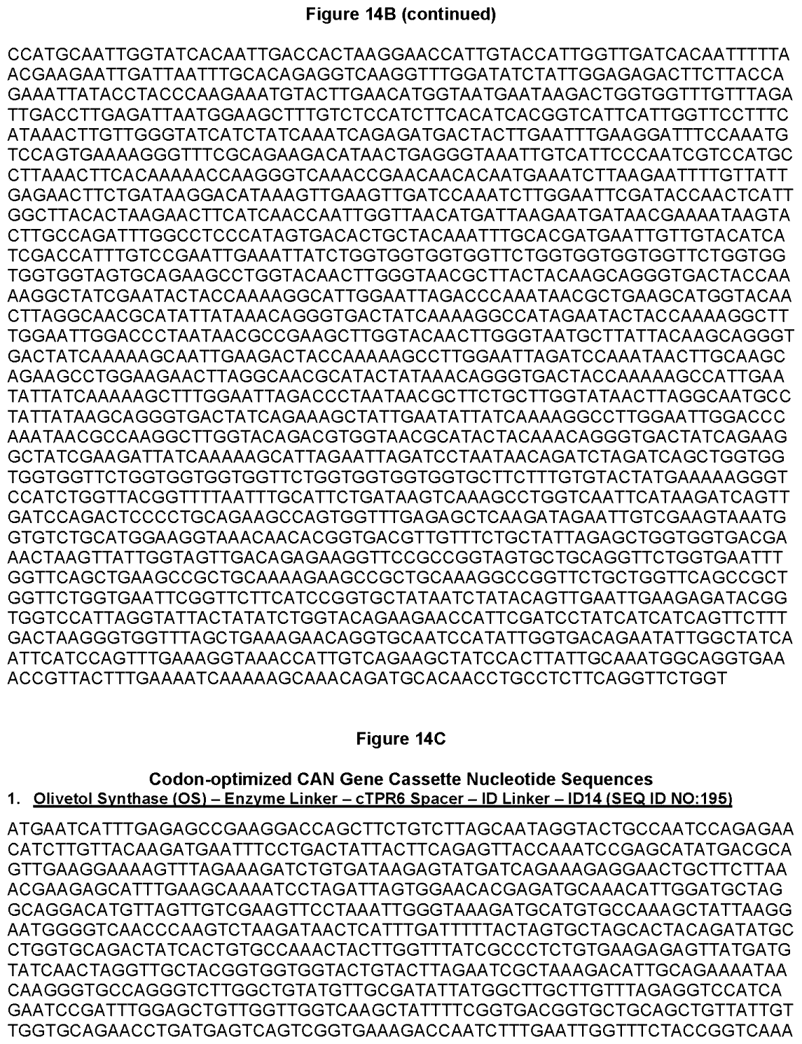

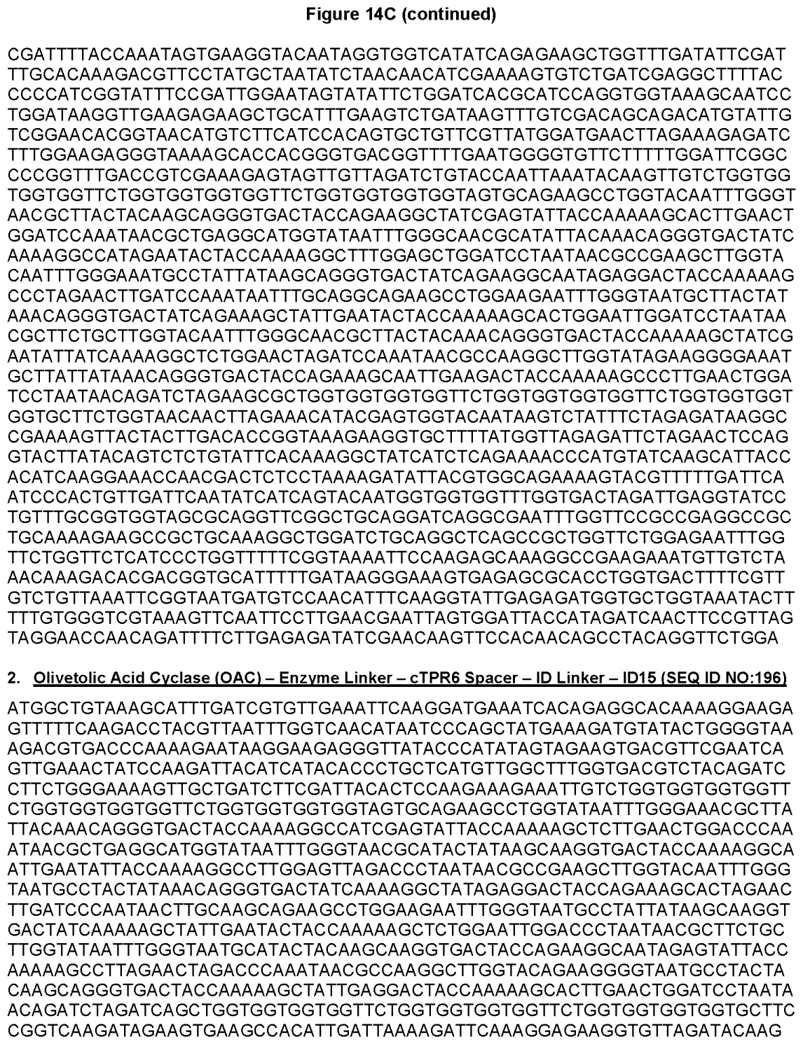

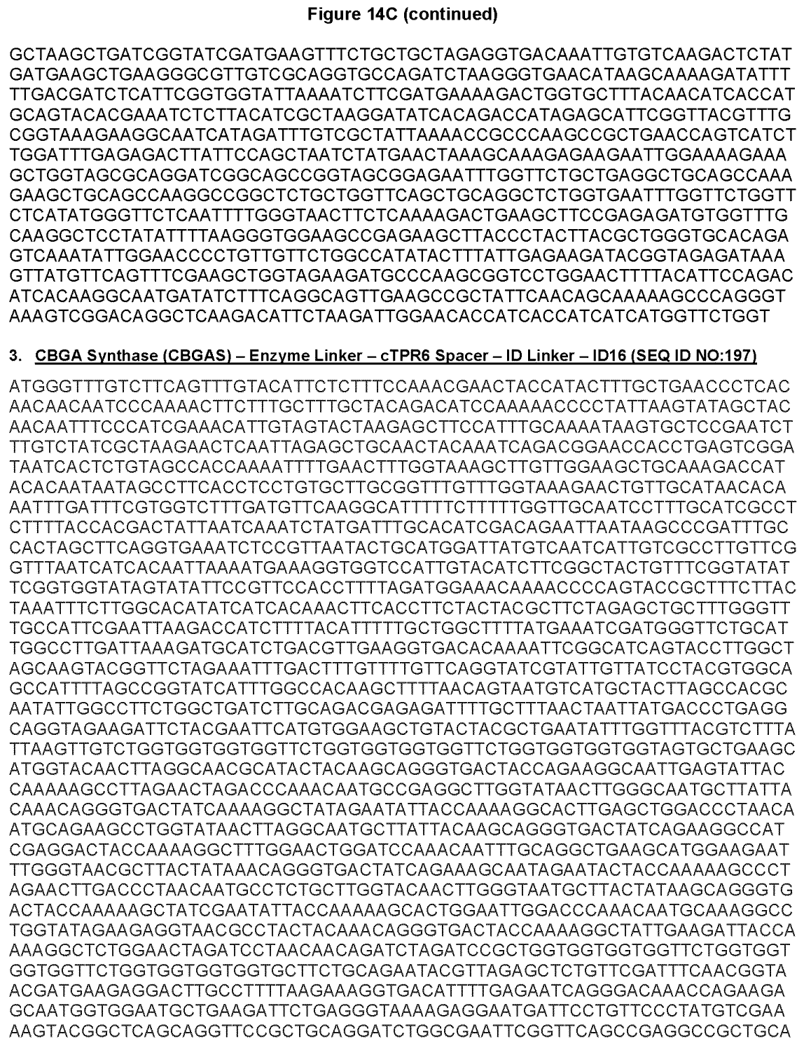

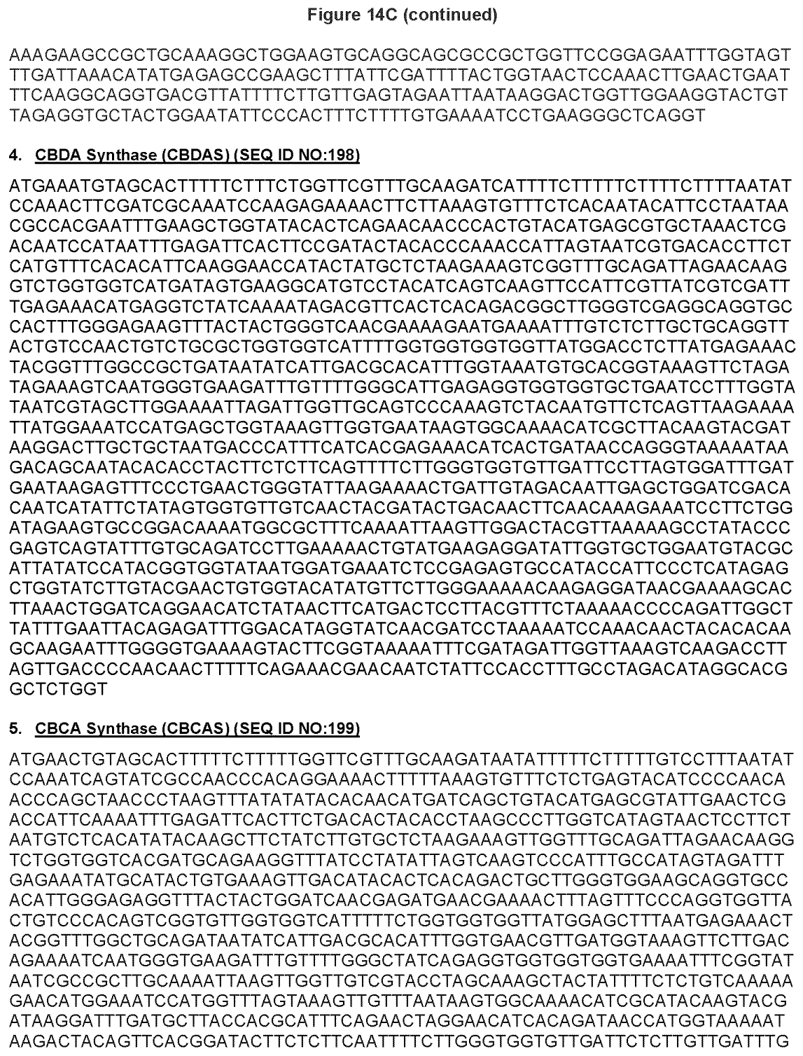

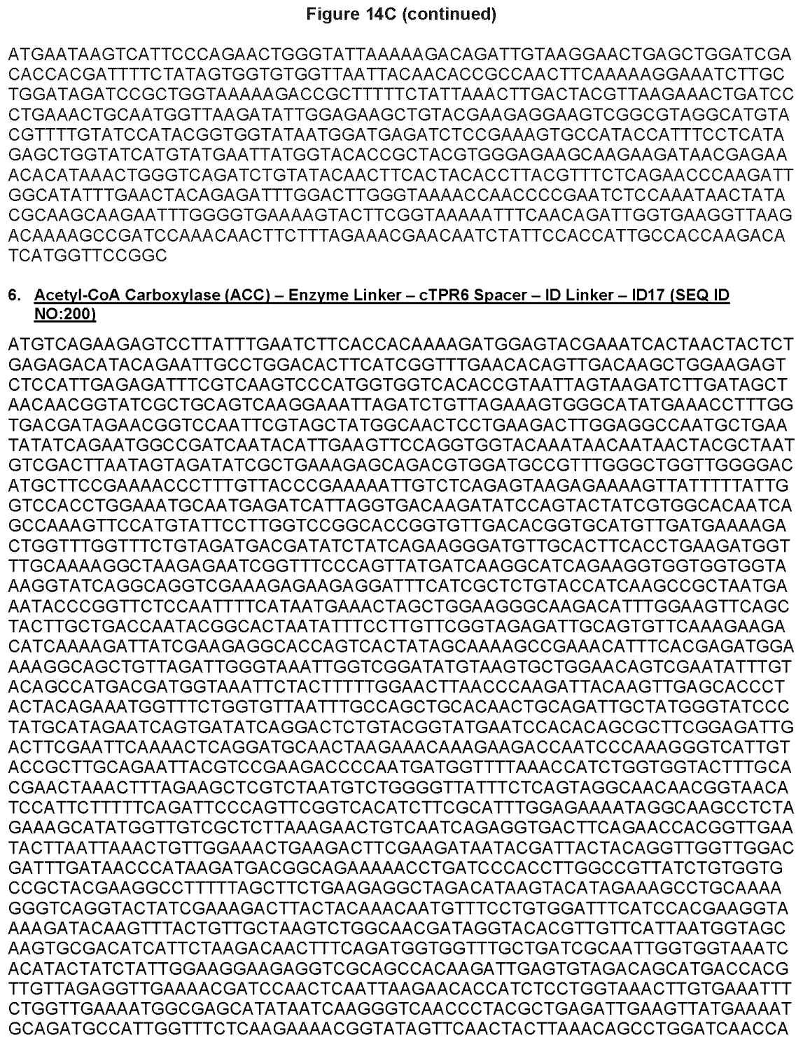

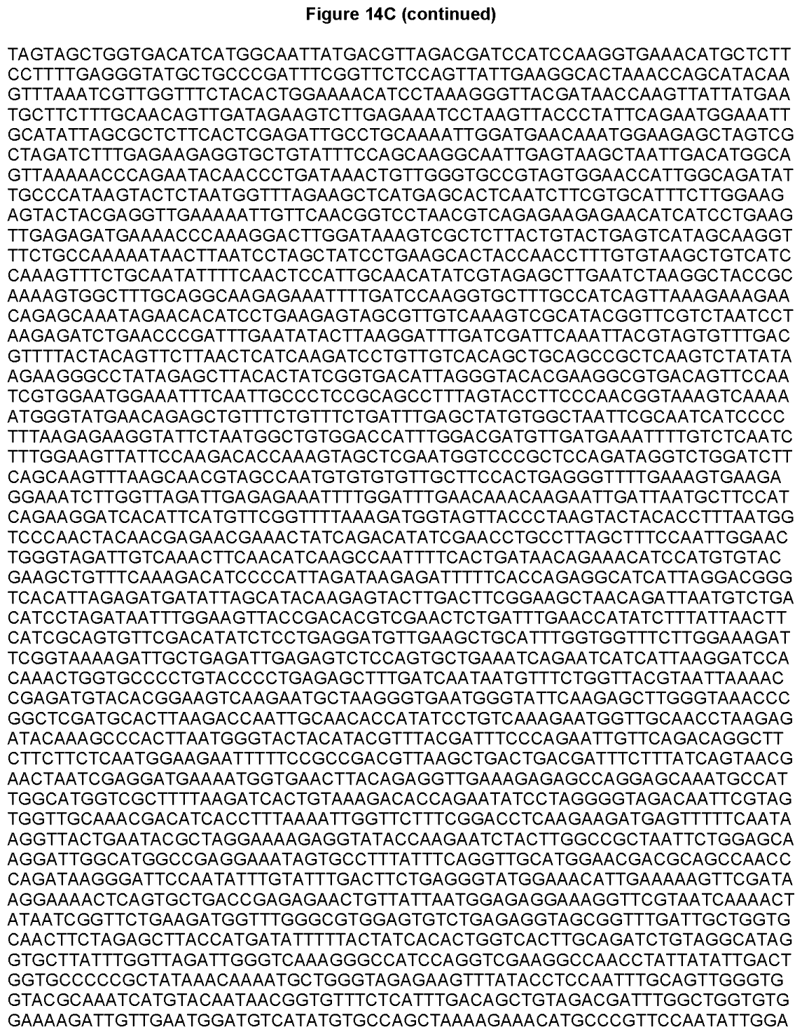

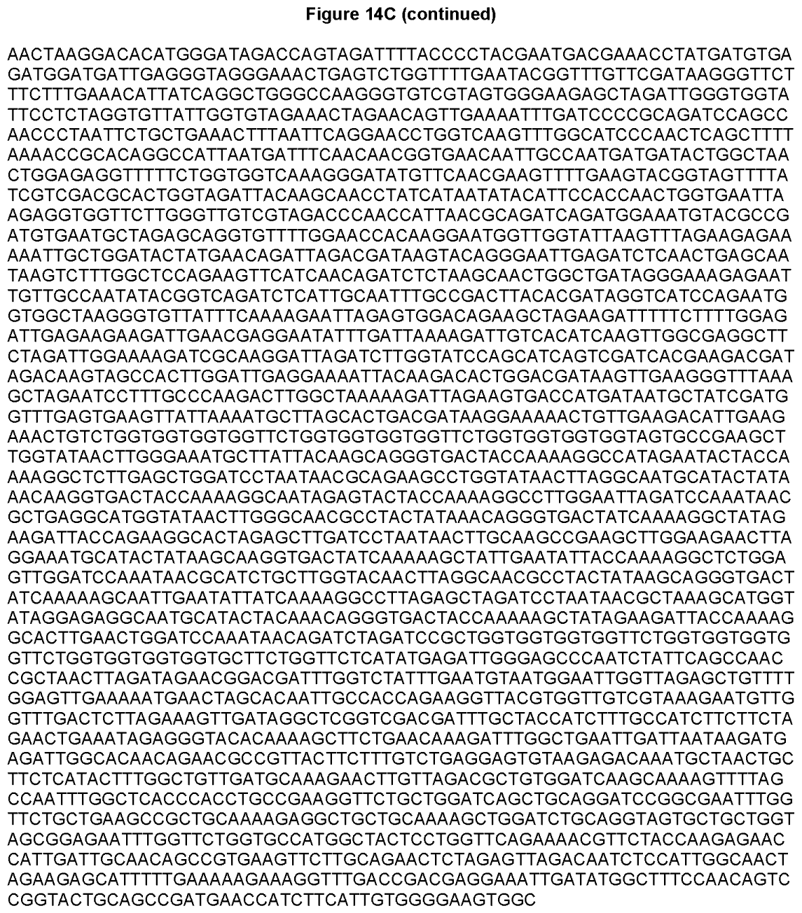

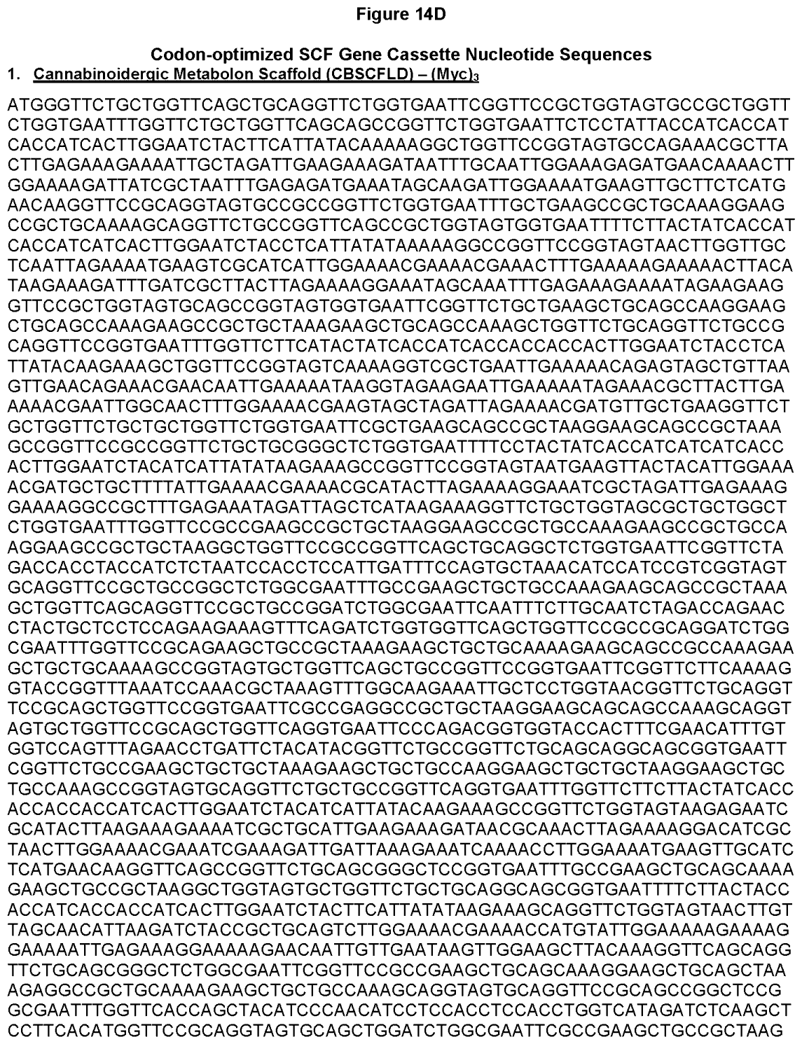

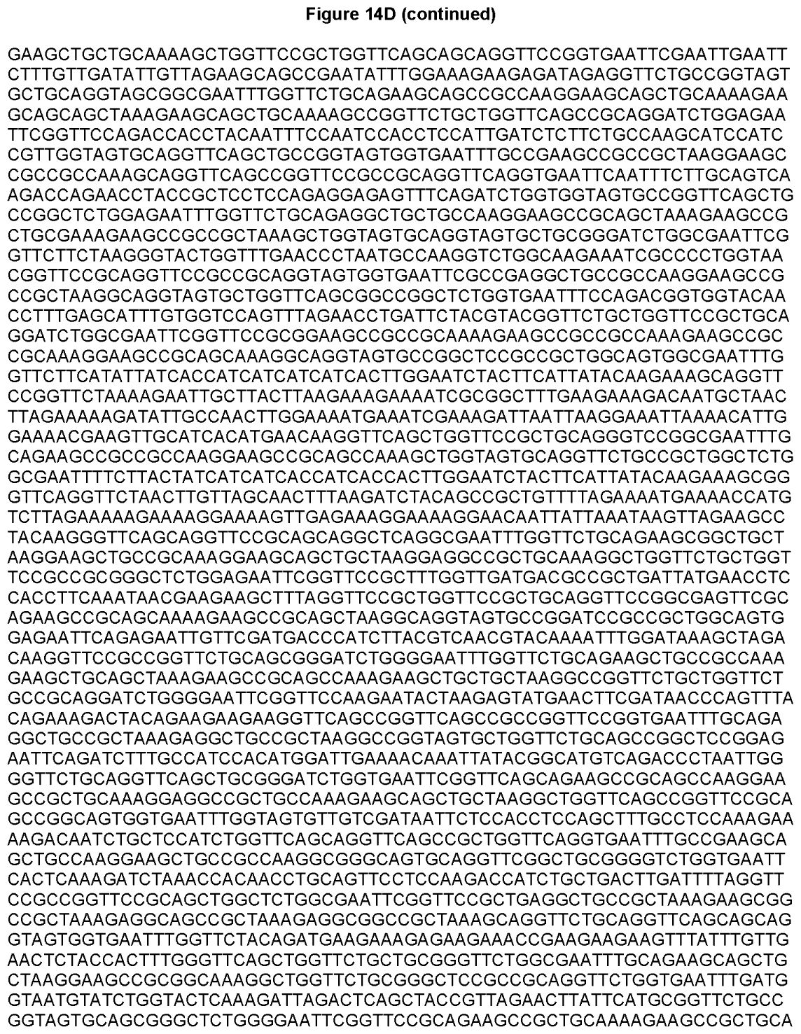

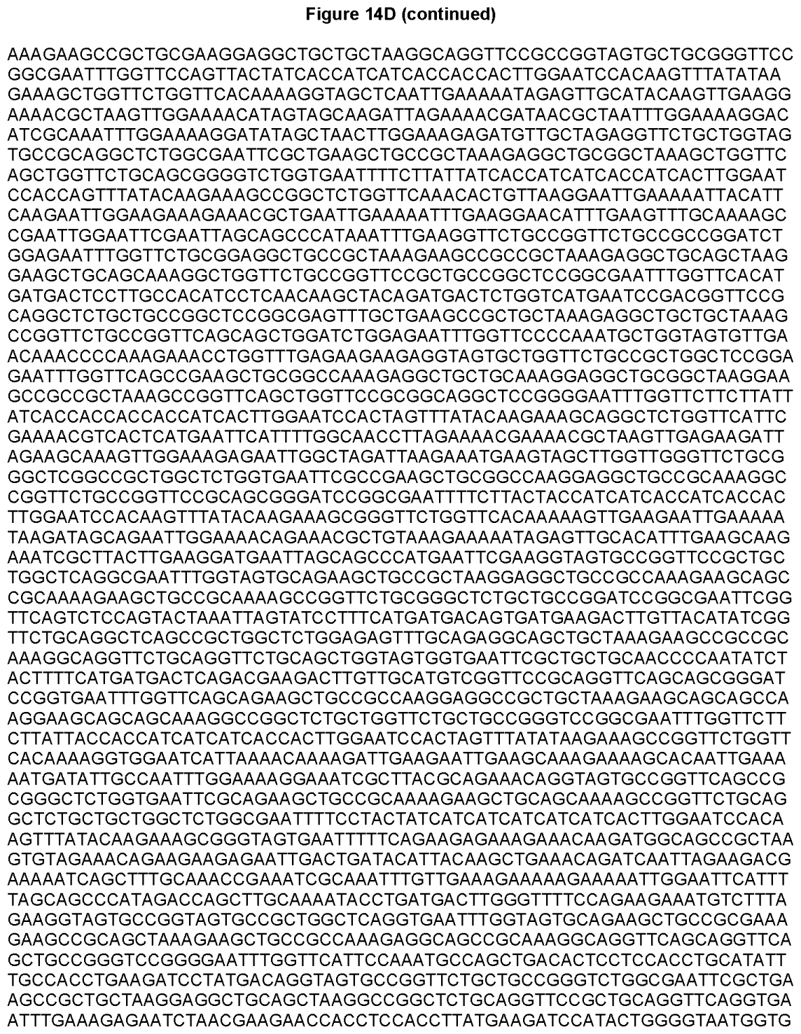

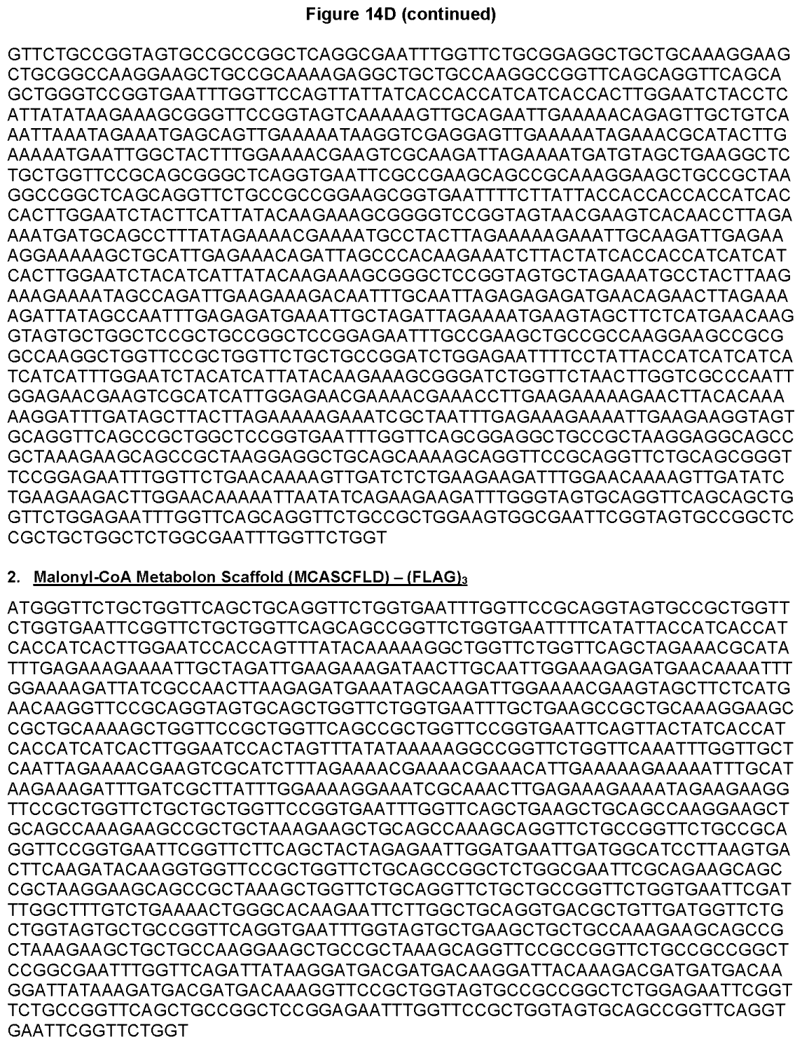

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) can be attained, using appropriate codon bias tables for that species. For example, the nucleotide sequences set forth in FIG. 12A are the nucleic acid sequences encoding an ATP citrate lyase, an atoB, a 3-hydroxbutyryl-CoA dehydrogenase, an enoyl-CoA hydratase, a beto-ketothiolase (bktB), a trans-enoyl-CoA reductase, an HMG-CoA synthase, an HMG-CoA reductase, a mevalonate kinase, a phosphomevalonate kinase, a diphosphomevalonate decarboxylase, an isopentenyl-diphosphate delta isomerase, a geranyl-diphosphate synthase (ERG20^{WW}), an olivetol synthase, an olivetolic acid cyclase, a CBGA synthase, a CBDA synthase, a CBCA synthase, an acetyl-CoA carboxylase, and a hexanoyl-CoA synthetase. The nucleic acid sequences for the ATP citrate lyase, atoB, 3-hydroxybutyryl-CoA dehydrogenase, enoyl-CoA hydratase, trans-enoyl-CoA reductase, bktB, olivetol synthase, olivetolic acid cyclase, CBGA synthase, CBDA synthase, and CBCA synthase have been codon optimized for expression in yeast. FIGS. 14A-14C contain codon optimized (for expression in yeast) nucleic acid sequences encoding the engineered enzymes of FIGS. 13A-13C.

In addition to sequence similarity, it will be appreciated that enzymes and scaffolds with structural and/or functional similarity to the enzymes and scaffolds described herein are also encompassed within the scope of the document.

This document provides recombinant host cells that can be used to produce one or more cannabinoids as described herein. For example, an individual host cell can contain exogenous nucleic acid such that the scaffold polypeptide and each of the enzymes to be immobilized on the scaffold are expressed. It is important to note that such host cells can contain any number and/or combination of exogenous nucleic acid molecules. For example, a particular host cell can contain an exogenous nucleic acid encoding the scaffold, and additional exogenous nucleic acids encoding the enzymes of the malonyl-CoA pathway, enzymes of the hexanoyl-CoA pathway or encoding a HCS, and enzymes of the mevalonate or MEP pathway. A single exogenous nucleic acid can encode one enzyme or more than one enzyme (e.g., one or more copies of from one to ten (or more) enzymes, from one to eight, from one to seven, from one to six, from one to five, from one to four, or from two to three enzymes). Thus, the number of different exogenous nucleic acids needed to produce the engineered enzymes to be localized on the scaffold will depend on the design of the scaffold and/or the particular embodiment. FIG. 2A and FIG. 2B each provide a non-limiting schematic of suitable gene cassettes for expressing the scaffolds and enzymes. FIG. 12C provides the nucleic acid sequence encoding a scaffold polypeptide containing the peptide ligands corresponding to IDs 1-16 as shown in Table 2 and a triplicate MYC tag. See also FIG. 14D for the codon-optimized nucleic acid sequence encoding the scaffold polypeptide of FIG. 13D. FIG. 12D provides the nucleic acid sequence encoding a scaffold polypeptide that contains the peptide ligands corresponding to IDs 1 and 17, and a triplicate FLAG tag. See also FIG. 14D.

In some embodiments, multiple nucleic acids encoding polypeptides (e.g., the nucleic acids of a gene cassette such as in FIG. 2A or FIG. 2B) can be linked together using a nucleic acid sequence encoding a self-cleaving peptide. During translation of the transcripts, the growing polypeptide can be cleaved at the 2A peptide with translation continuing through to the next polypeptide. When designing a vector to express the polypeptides as a polycistronic unit, the nucleic acid encoding the polypeptides and the self-cleaving peptide (e.g., a 2A peptide) can be designed such that they are in translational frame with each other. Examples of 2A peptides that can be used as described herein include, without limitation, a 2A peptide of foot-and-mouth disease virus (FMDV), a 2A peptide of equine rhinitis A virus (ERAVO), a 2A peptide of Thosea asigna virus (TaV), or a 2A peptide of porcine teschovirus-1 (PTV-1) or porcine teschovirus-2 (PTV-2). The 2A peptides from PTV-1 and PTV-2 are referred to as P2A peptides. See, e.g., SEQ ID NO:212 for a codon-optimized nucleotide sequence (for S. cerevisiae) encoding a P2A peptide.

Further, the cells described herein can contain a single copy or multiple copies (e.g., about 5, 10, 20, 35, 50, 75, 100 or 150 copies), of a particular exogenous nucleic acid molecule. Again, the cells described herein can contain more than one particular exogenous nucleic acid molecule and/or copies thereof. For example, a particular cell can contain about 50 copies of exogenous nucleic acid molecule X as well as about 75 copies of exogenous nucleic acid molecule Y.

Any method can be used to introduce an exogenous nucleic acid molecule into a host cell. In fact, many methods for introducing nucleic acid into host cells such as bacteria and yeast are well known to those skilled in the art. For example, heat shock, lipofection, electroporation, nucleofection, conjugation, fusion of protoplasts, and biolistic delivery are common methods for introducing nucleic acid into bacteria and yeast cells. See, e.g., Ito et al., J. Bacterol. 153:163-168 (1983); Durrens et al., Curr. Genet. 18:7-12 (1990); and Becker and Guarente, Methods in Enzymology 194:182-187 (1991).

An exogenous nucleic acid molecule contained within a particular host cell can be maintained within that host cell in any form. For example, exogenous nucleic acid molecules can be integrated into the genome of the microorganism or maintained in an episomal state. In other words, a microorganism can be a stable or transient transformant. Again, a microorganism described herein can contain a single copy, or multiple copies (e.g., about 5, 10, 20, 35, 50, 75, 100 or 150 copies), of a particular exogenous nucleic acid molecule as described herein.

Suitable nucleic acid constructs for expressing the engineered enzymes and scaffolds include, for example, CRISPR plasmids, baculovirus vectors, bacteriophage vectors, plasmids, phagemids, cosmids, fosmids, bacterial artificial chromosomes, viral vectors (for example, viral vectors based on vaccinia virus, poliovirus, adenovirus, adeno-associated virus, SV40, herpes simplex virus, and the like), P1-based artificial chromosomes, yeast plasmids, yeast artificial chromosomes, and other vectors. Typically such constructs include a regulatory element that promotes the expression of a nucleic acid sequence that encodes a polypeptide. Typically, regulatory elements are DNA sequences that regulate the expression of other DNA sequences at the level of transcription. Thus, regulatory elements include, without limitation, promoters, enhancers, and the like. Any type of promoter can be used to express an amino acid sequence from an exogenous nucleic acid molecule. Examples of promoters include, without limitation, constitutive promoters, tissue-specific promoters, and inducible or repressible promoters that are responsive or unresponsive to a particular stimulus (e.g., light, oxygen, chemical concentration, sound, and the like).

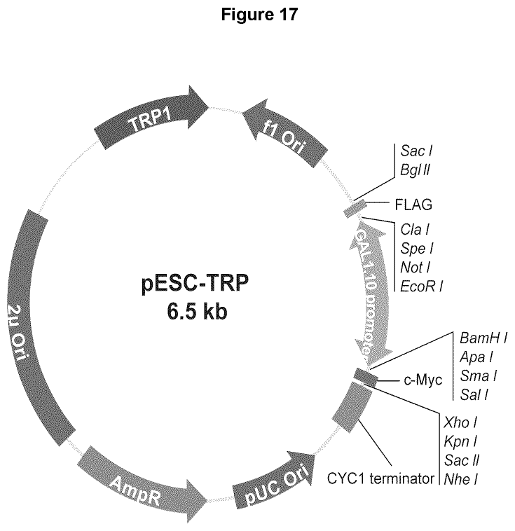

In some embodiments, endogenous yeast promoters with varying constitutive activity levels can be used to express the engineered enzymes and/or scaffolds. To maintain an excess of enzymes relative to scaffold molecules, the scaffolds can be expressed under control of the weakest promoter. For example, one or more of the following yeast promoters can be used: the promoter from the gene encoding transcriptional elongation factor EF-1α (pTEF1), the promoter from the gene encoding phosphoglycerate kinase (PGK1), the promoter from the gene encoding triose phosphate isomerase (pTPI1), the promoter from the gene encoding a hexose transporter (pHXT7), HXT7, the promoter from the gene encoding pyruvate kinase 1 (pPYK1), the promoter from the gene encoding alcohol dehydrogenase 1 (pADH1), or the promoter from the gene encoding triphosphate dehydrogenase (pTDH3). For example, in the embodiment shown in FIG. 2A, the pTPI1 promoter can be used to express enzymes of the upper hexanoyl-CoA (HCA), enzymes of the lower HCA pathway, enzymes of the upper mevalonate (MVA) pathway, enzymes of the lower MVA pathway, and enzymes of the lower cannabinoid (CB) pathway, while the pTEF1 promoter can be used to express enzymes of the upper CB pathway, the atoB enzyme, and the enzymes of the malonyl-CoA pathway, and the pADH1 promoter can be used to express the scaffold. Of these promoters, the pADH1 promoter has the weakest activity (+ in FIG. 2A), the pTEF1 promoter has the strongest activity (+++ in FIG. 2A), and the activity of the pTPI1 promoter is between the other two (++ in FIG. 2A). In some embodiments, the Gal 1-10 promoter (e.g., from S. cerevisiae) can be used. See, e.g., FIG. 17.

A nucleic acid construct also can include a selectable marker, e.g., for an antibiotic such as neomycin resistance, ampicillin resistance, tetracycline resistance, chloramphenicol resistance, or kanamycin resistance). In some embodiments, a nutritional marker gene that confers prototrophy for an essential nutrient such as tryptophan (TRP1), uracil (URA3), histidine (HIS3), leucine (LEU2), lysine (LYS2), or methionine can be included on a nucleic acid construct. See, e.g., FIG. 17. As shown in Example 3, four different auxotrophic markers were used to sequentially select for transformed cells containing the desired combinations of nucleic acids encoding the enzymes and scaffold. For example, yeast cells transformed with a vector containing a TRP gene and the nucleic acids encoding enzymes of the hexanoyl-CoA pathway were grown in tryptophan deficient media. The transformed cells that grew in the tryptophan deficient media were selected and further transformed with a vector containing a LEU gene and nucleic acid encoding enzymes of the mevalonate pathway. The resulting transformed cells were grown on media lacking tryptophan and leucine, and the cells that grew in the media lacking tryptophan and leucine were transformed with a vector containing a HIS gene and nucleic acids encoding enzymes of the upper cannabinoid pathway. The resulting transformed cells were grown on media lacking tryptophan, leucine, and histidine, and the cells that grew in the media lacking tryptophan, leucine, and histidine were transformed with a vector containing a URA3 gene and a nucleic acid encoding a scaffold. The resulting transformed cells were grown on media lacking tryptophan, leucine, histidine, and uracil. Cells that grew in media lacking tryptophan, leucine, histidine, and uracil contained the desired combination of enzymes and scaffold as shown in FIG. 1B.

In some embodiments, the encoded enzymes (e.g., one or more enzymes from the cannabinoid biosynthesis pathway, mevalonate pathway, MEP pathway, hexanoyl-CoA pathway, or a hexanoyl-CoA synthetase) and/or the scaffold can include a targeting sequence that can be used to direct the enzymes or scaffold to one of several different intracellular compartments, including, for example, the endoplasmic reticulum (ER), mitochondria, plastids (such as chloroplasts), the vacuole, the Golgi apparatus, or protein storage vesicles (PSV). For example, a mitochondrial or plastidial targeting sequence can be used to facilitate mitochondrial or plastidial compartmentalization of cannabinoid/cannabinoid precursor biosynthesis such that the encoded enzymes and scaffold are expressed in the mitochondria or plastids of the host cell.

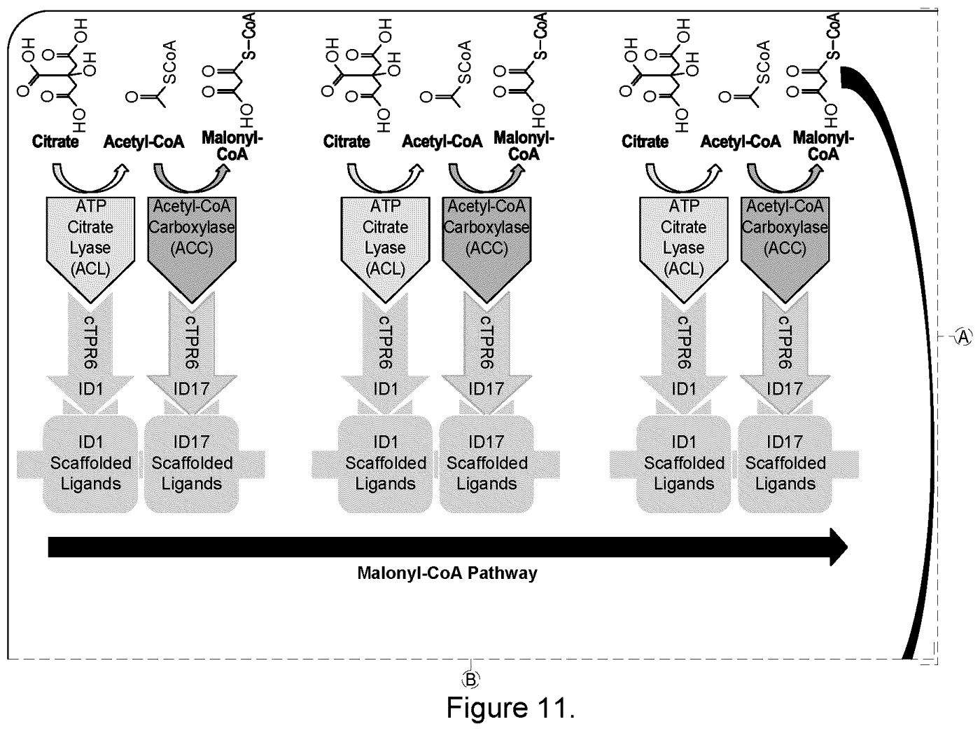

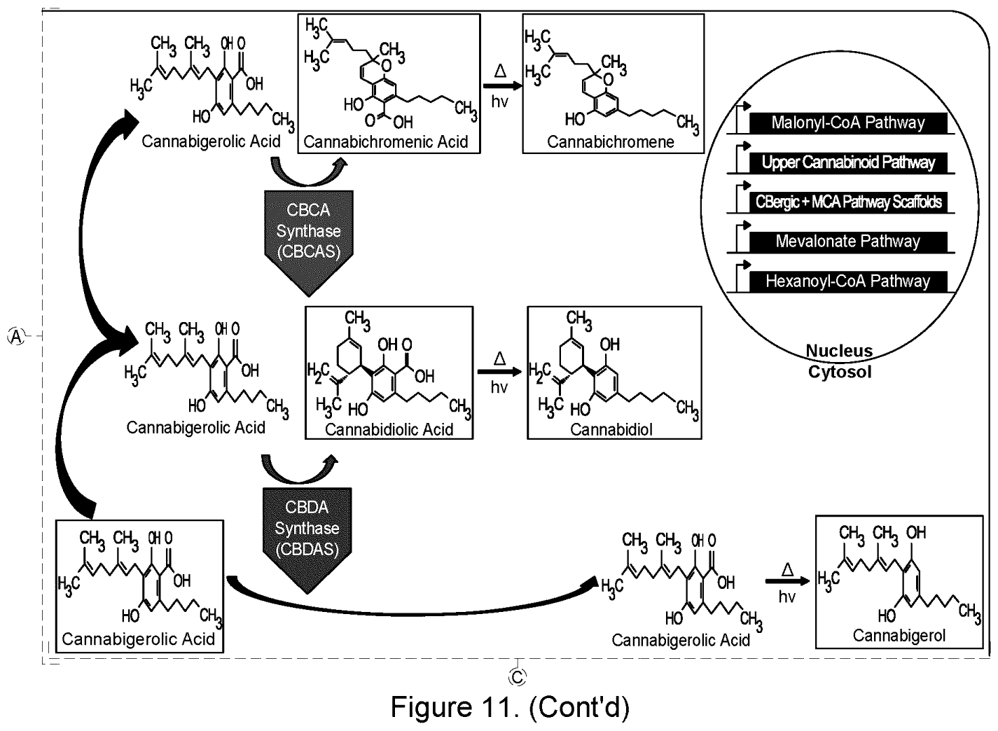

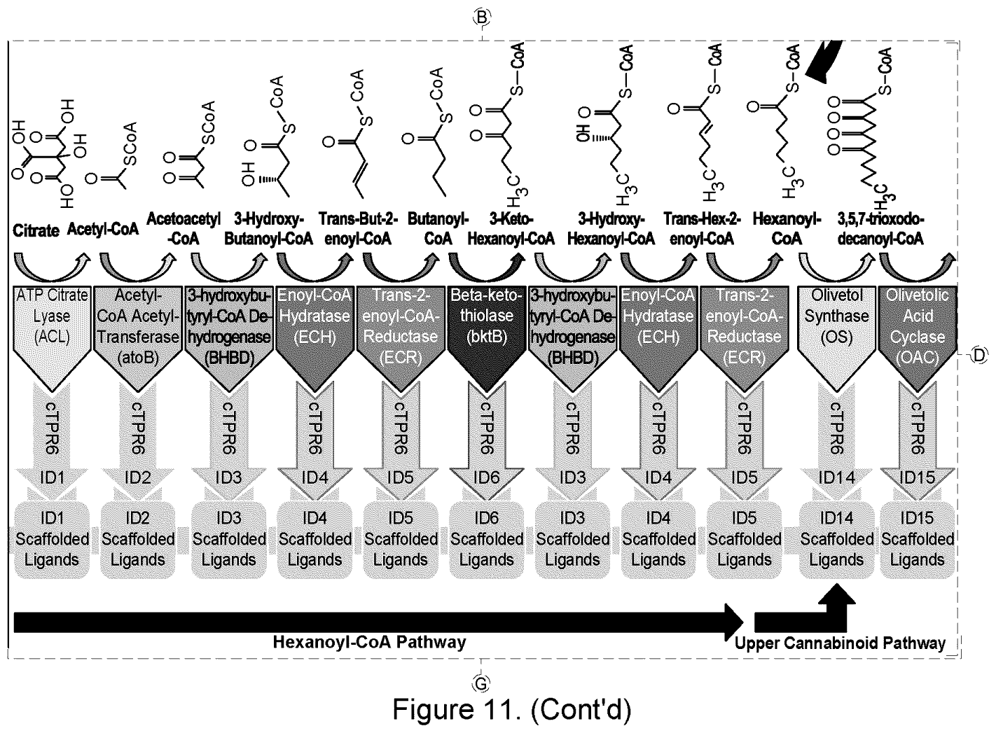

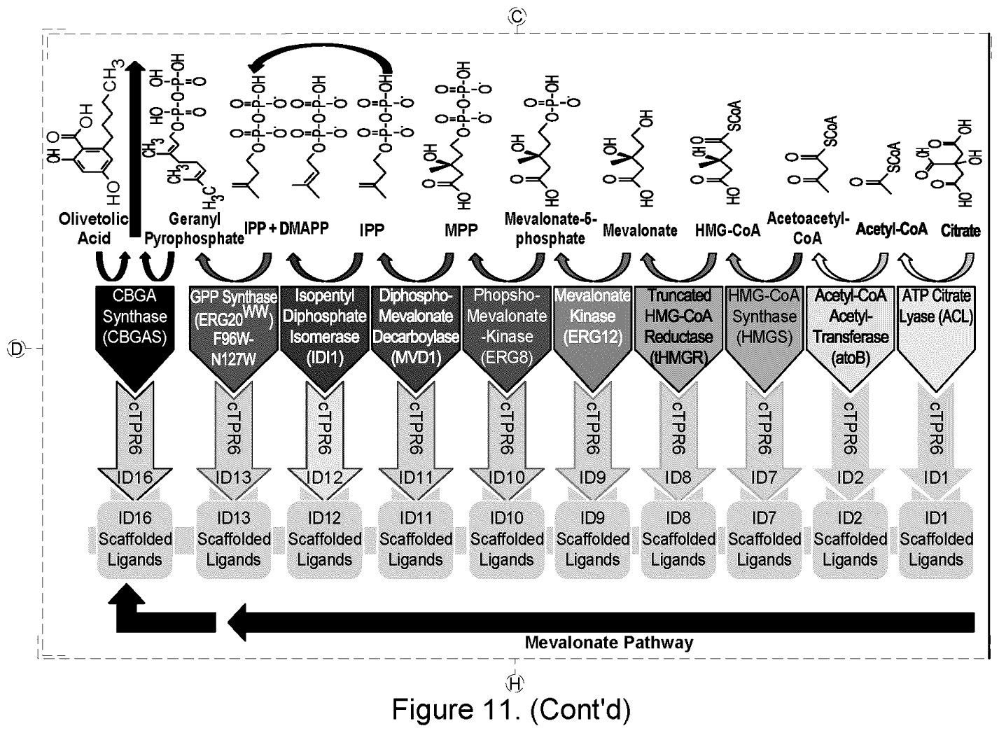

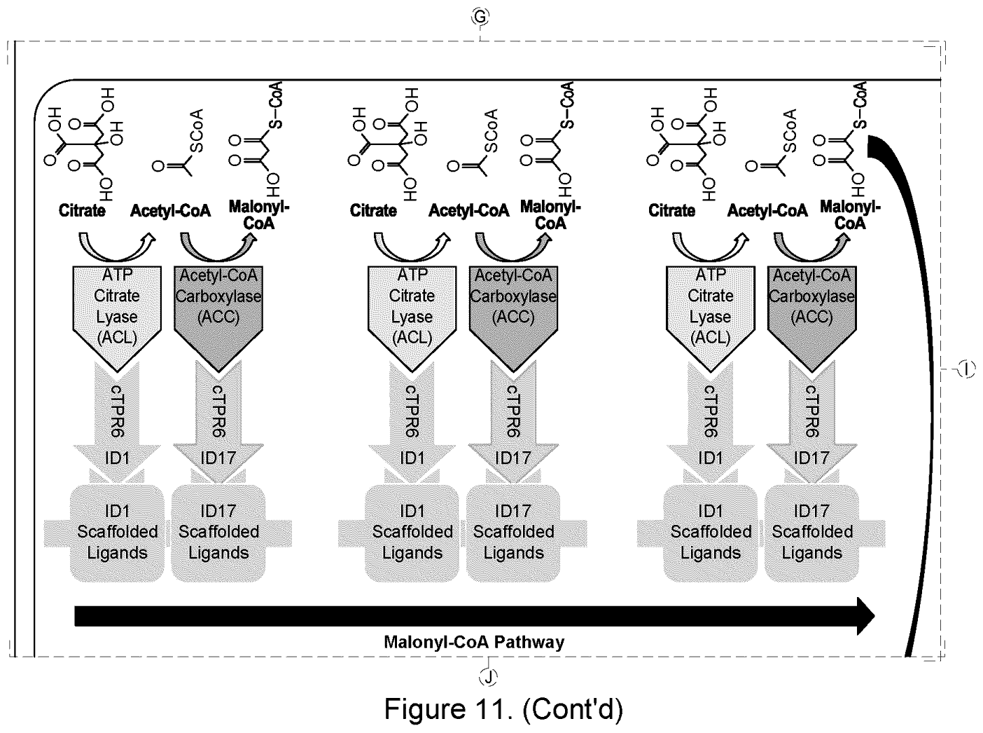

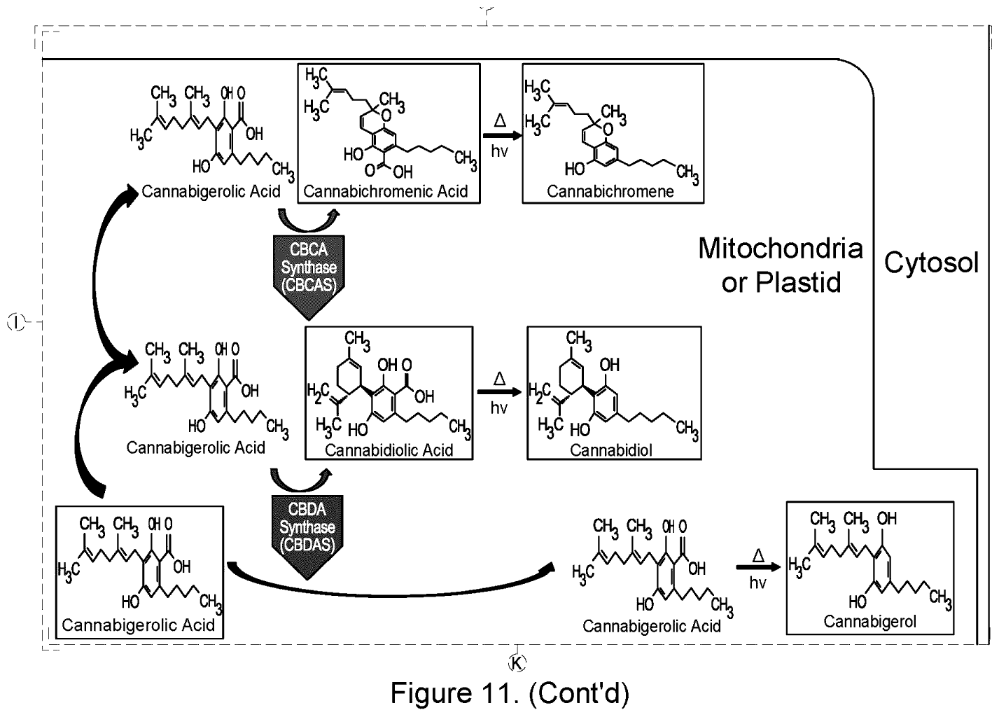

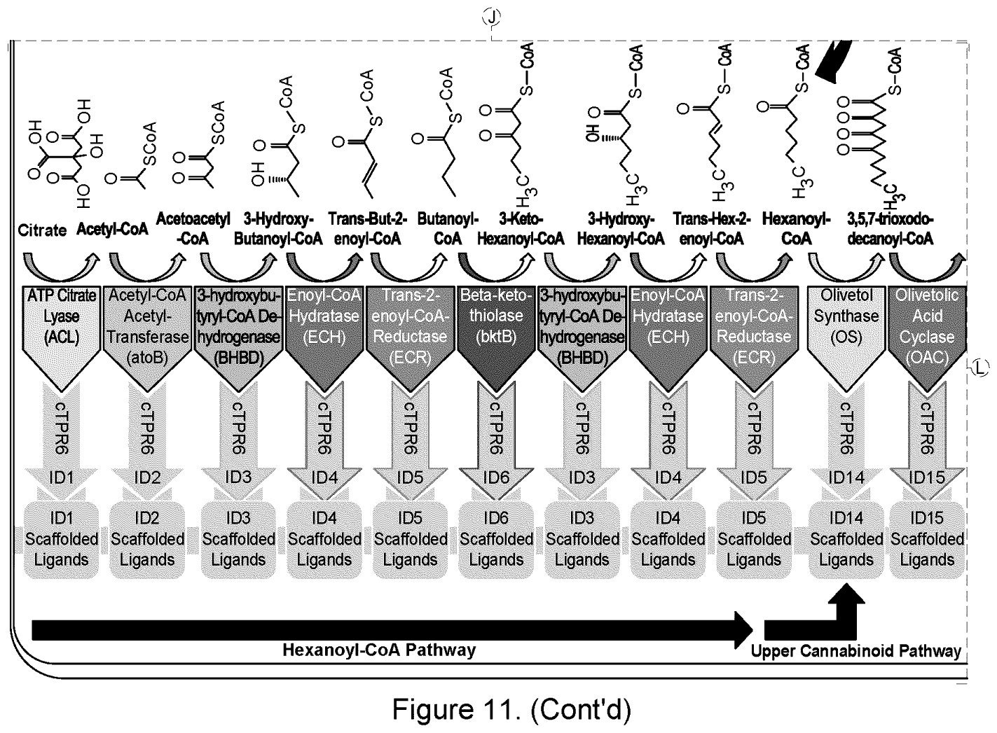

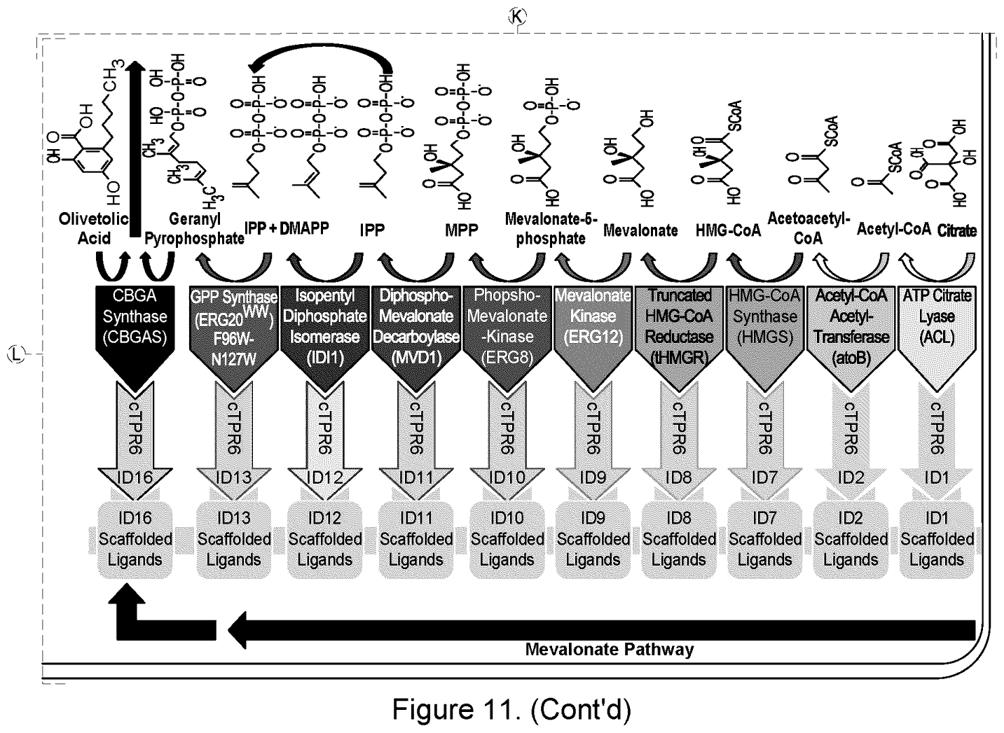

In some embodiments, cannabinoid/cannabinoid precursor biosynthesis can be performed in two compartments by co-expressing one or more engineered enzymes and a scaffold in both the cytosolic compartment and either the plastids or mitochondria of the host cell. See, for example, FIG. 11. It will be appreciated that while FIG. 11 depicts a scaffold containing enzymes of the hexanoyl-CoA pathway, enzymes of the upper cannabinoid pathway, and enzymes of the mevalonate pathway, dual-compartment engineering can be performed with any of the scaffolds and enzymes described herein. For example, dual-compartment engineering can be performed in two compartments by co-expressing a scaffold and enzymes of the hexanoyl-CoA pathway, enzymes of the upper cannabinoid pathway, and enzymes of the MEP pathway in both the cytosolic compartment and either the plastids of mitochondria of the host cell. Dual-compartment engineering also can be achieved by engineering separate haploid yeast strains for cytosolic and mitochondrial/plastidial cannabinoid biosynthesis, and then mating these two haploid strains to produce a diploid lineage that is heterozygous for cytosolic and mitochondrial/plastidial cannabinoid biosynthesis.

In some embodiments, the engineered enzymes and/or scaffolds also contain a tag that can be used for purification of the recombinant protein (e.g., c-myc, FLAG, polyhistidine (e.g., hexahistidine), hemagglutinin (HA), glutathione-S-transferase (GST), or maltose binding protein (MBP)) or as a detectable marker (e.g., luciferase, green fluorescent protein (GFP), or chloramphenicol acetyl transferase (CAT)). For example, in the embodiment shown in FIG. 6C and FIG. 6D, a scaffold can include a myc tag (e.g., (Myc)₃ tag) or a FLAG tag (FLAG)₃ tag at the C-terminus.

In some embodiments, a host cell can be engineered to increase acetyl-CoA availability for cannabinoid and cannabinoid precursor biosynthesis. For example, the mitochondrial enzyme isocitrate dehydrogenase-1 (IDH1) can be placed under transient micro-RNA-mediated inducible repression. Since mitochondrial IDH1 is primarily responsible for depletion of the cellular citrate pool, micro-RNA-mediated repression of IDH1 can increase the availability and cytosolic shuttling of citrate for production of acetyl-CoA by ATP citrate lyase. The resulting increase in acetyl-CoA bioavailability can further enhance downstream hexanoyl-CoA and geranyl pyrophosphate titers by improving initial substrate availability for the hexanoyl-CoA and mevalonate pathways. The combinatorial metabolic engineering of acetyl-CoA can mitigate issues related to the siphoning of acetyl-CoA away from the endogenous metabolism of the host cells.

In some embodiments, one or more conventional and/or contemporary gene editing techniques can be used to produce recombinant hosts. For example, clustered, regularly interspaced, short palindromic repeat (CRISPR) technology can be used to modify expression of an endogenous nucleic acid. The CRISPR/Cas system includes components of a prokaryotic adaptive immune system that is functionally analogous to eukaryotic RNA interference, using RNA base pairing to direct DNA or RNA cleavage. The Cas9 protein functions as an endonuclease, and CRISPR RNA (crRNA) and trans-activating RNA (tracrRNA) sequences complex with the Cas9 enzyme and direct it to a target DNA sequence (Makarova et al., Nat Rev Microbiol 9(6): 467-477, 2011). The modification of a single targeting RNA can be sufficient to alter the nucleotide target of a Cas protein. In some cases, crRNA and tracrRNA can be engineered as a single cr/tracrRNA hybrid (also referred to as a “guide RNA” or “gRNA”) to direct Cas9 cleavage activity (Jinek et al., Science, 337(6096):816-821, 2012). The CRISPR/Cas system can be used in a variety of prokaryotic and eukaryotic organisms (see, e.g., Jiang et al., Nat Biotechnol, 31(3):233-239, 2013; Dicarlo et al., Nucleic Acids Res, doi:10.1093/nar/gkt135, 2013; Cong et al., Science, 339(6121):819-823, 2013; Mali et al., Science, 339(6121):823-826, 2013; Cho et al., Nat Biotechnol, 31(3):230-232, 2013; and Hwang et al., Nat Biotechnol, 31(3):227-229, 2013).

Another gene-editing technique can include a sequence-specific nuclease created by fusing transcription activator-like effectors (TALEs) to, for example, the catalytic domain of the Fold endonuclease. Both native and custom TALE-nuclease (“TALEN”) fusions direct DNA double-strand breaks to specific, targeted sites. See, for example, Christian, et al., Genetics 186:757-761 (2010) and U.S. Patent Publication No. 20110145940.

Other suitable gene insertion techniques include the use of retroviral vectors and biolistic particle gene delivery systems (colloquially known as “gene guns”).

Methods of identifying and/or selecting host cells that contain exogenous nucleic acid or a modified endogenous nucleic acid are well known to those skilled in the art. Such methods include, without limitation, the introduction and expression of a negative selection marker such as an antibiotic resistance gene, PCR, and nucleic acid hybridization techniques such as Northern and Southern analyses. In some cases, immunohistochemistry and biochemical techniques can be used to determine if a microorganism contains a particular nucleic acid by detecting the expression of the encoded enzymatic polypeptide encoded by that particular nucleic acid molecule. For example, an antibody having specificity for an encoded enzyme can be used to determine whether or not a particular cell contains that encoded enzyme. Further, biochemical techniques can be used to determine if a cell contains a particular nucleic acid molecule encoding an enzymatic polypeptide by detecting an organic product produced as a result of the expression of the enzymatic polypeptide.

This document also provides isolated nucleic acids molecules. The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

In some embodiments, the production of one or more cannabinoids can be performed in vitro using the scaffold and immobilized enzymes described herein, using a lysate (e.g., a buffered cell lysate) from a recombinant host cell as a source of the scaffold and enzymes, using a plurality of lysates from different host cells as the source of the scaffold and enzymes, or using an acellular reaction buffer such as a synthetic reaction buffer. For example, following co-immunoprecipitation of C-terminal Myc/Flag-tagged enzyme-bound scaffolds, scaffold-enzyme complexes can be maintained in a citrate-supplemented and/or glucose-supplemented (or other carbon source-supplemented) reaction buffer which allows in-vitro scaffolded cannabinoid biosynthesis.

### Producing Cannabinoids Using a Recombinant Host

Typically, one or more cannabinoids can be produced by providing a recombinant host such as a recombinant microorganism and culturing the microorganism with a culture medium. In general, the culture media and/or culture conditions can be such that the microorganisms grow to an adequate density and produce cannabinoids efficiently. For example, the microorganisms can be subjected to aerobic batch fermentation. In some embodiments, one or more precursors (e.g., citrate, glucose, hexanoic acid, and/or other carbon source and/or malonyl-CoA) are supplemented in the culture medium. In some embodiments, about 30 mg/L to about 10,000 mg/L (e.g., about 100 mg/L to about 5,000 mg/L, about 200 mg/L to about 4,000 mg/L, about 300 mg/L to about 3,000 mg/L, or about 350 mg/L to about 1,000 mg/L) of buffered citrate, pH 6.0 can be added to the culture medium.

For large-scale production processes, any method can be used such as those described elsewhere (Manual of Industrial Microbiology and Biotechnology, 2^{nd} Edition, Editors: A. L. Demain and J. E. Davies, ASM Press; and Principles of Fermentation Technology, P. F. Stanbury and A. Whitaker, Pergamon). Briefly, a large vessel (e.g., a 100 gallon, 200 gallon, 500 gallon, or higher volume vessel) containing an appropriate culture medium is inoculated with a particular microorganism. After inoculation, the microorganism is incubated to allow biomass to be produced. Once a desired biomass or cellular confluency is attained, a portion or all of the broth containing the microorganisms can be transferred to a second vessel. This second vessel can be any size. For example, the second vessel can be larger, smaller, or the same size as the first vessel. Typically, the second vessel is larger than the first such that additional culture medium can be added to the broth from the first vessel. In addition, the culture medium within this second vessel can be the same as, or different from, that used in the first vessel. This system can expand to include an array consisting of any number of individual vessels.

Once transferred, the microorganisms can be incubated to allow for the production of one or more cannabinoids. Once produced, any method can be used to isolate cannabinoids. For example, common separation techniques can be used to remove the biomass from the broth, and common isolation procedures (e.g., extraction such as non-polar extraction with hexane followed by ethyl-acetate), high-performance liquid chromatography (e.g., HPLC with a diode array detector (HPLC-DAD)), gas chromatography-flame ionization detection (GC-FID), or ion-exchange procedures) can be used to obtain the cannabinoids from the biomass.

A host cell described herein can produce one or more cannabinoids at a concentration of at least about 10 mg per L (e.g., at least about 15 mg/L 25 mg/L, 50 mg/L, 75 mg/L, 100 mg/L, 150 mg/L, 200 mg/L, 250 mg/L or more). For example, in some embodiments, total cannabinoids (total of CBG, CBGA, CBD, CBDA, CBC, and CBCA) can be produced at a concentration of at least about 10 mg/L, 15 mg/L, 20 mg/L, 40 mg/L, 60 mg/L, 80 mg/L, or 100 mg/L or more. For example, in some embodiments, total cannabinoids (total of CBG, CBGA, CBD, CBDA, CBC, and CBCA) can be produced at a concentration from about 10 mg/L to about 500 mg/L (e.g., 20 mg/L to 450 mg/L, 40 mg/L to 380 mg/L, 60 mg/L to 280 mg/L, 60 mg/L to 250 mg/L, 60 mg/L to 150 mg/L, 80 mg/L to 400 mg/L, 80 mg/L to 300 mg/L, 80 mg/L to 250 mg/L, 80 mg/L to 200 mg/L, 80 mg/L to 175 mg/L, 90 mg/L to 400 mg/L, 90 mg/L to 300 mg/L, 90 mg/L to 250 mg/L, or 90 mg/L to 150 mg/L). In some embodiments, one or more individual cannabinoids (e.g., one or more of CBG, CBGA, CBD, CBDA, CBC, and CBCA) can be produced at concentrations of at least about 1 mg/L, 2 mg/L, 5 mg/L, 10 mg/L, 15 mg/L, 20 mg/L, 25 mg/L, 30 mg/L, 35 mg/L, 40 mg/L, 45 mg/L, 50 mg/L, 55 mg/L, 60 mg/L, 65 mg/L, 70 mg/L, 75 mg/L, 80 mg/L, 85 mg/L, 90 mg/L, 95 mg/L, 100 mg/L or more. For example, in some embodiments, one or more individual cannabinoids can be produced at a concentration from about 1 mg/L to about 100 mg/L (e.g., 2 to 90 mg/L, 2 to 80 mg/L, 2 to 70 mg/L, 2 to 60 mg/L, 2 to 50 mg/L, 2 to 40 mg/L, 2 to 30 mg/L, 2 to 20 mg/L, 2 to 15 mg/L, 3 to 90 mg/L, 3 to 80 mg/L, 3 to 70 mg/L, 3 to 60 mg/L, 3 to 50 mg/L, 3 to 40 mg/L, 3 to 30 mg/L, 3 to 20 mg/L, 3 to 15 mg/L, 4 to 90 mg/L, 4 to 80 mg/L, 4 to 70 mg/L, 4 to 60 mg/L, 4 to 50 mg/L, 4 to 40 mg/L, 4 to 30 mg/L, 4 to 20 mg/L, or 4 to 15 mg/L).

The invention will be further described in the following examples, which do not limit the scope of the invention described in the claims.

### EXAMPLES

### Example 1—General Methods

### Enzymatic Constructs

Each enzyme construct is designed to include an interaction domain (ID) which is comprised of two tandem N-terminal or C-terminal ligand-binding motifs which are separated from the given enzyme and from one another by an amino acid sequence containing flexible GS-rich linkers flanking a rigid α-helical spacer sequence. The motifs comprising the ID of each enzyme specifically bind tandem peptide ligands which form ID-binding sites at discrete locations along a synthetic intracellular polypeptide scaffold. Expression of each enzyme is controlled by a constitutive or inducible promoter. The nucleic acid encoding the enzyme can be codon optimized, e.g., for expression in yeast.

### Scaffolding Constructs

ID-binding sites containing tandem peptide ligands that are specific for the tandem scaffold-binding motifs, which comprise the ID of each enzyme, are inserted at discrete positions along an intracellular polypeptide scaffold.

The tandem ligands which comprise each scaffolded ID-binding site are separated from one another by a 36 amino acid residue sequence containing flexible GS-rich linkers flanking a rigid α-helical spacer sequence, while the scaffolded ID-binding sites themselves are separated from one another by a 50 amino acid residue sequence (or any other number of amino acid residues) containing flexible GS-rich linkers flanking a rigid α-helical spacer sequence. Specifically, the scaffold binding sites for each enzyme in the hexanoyl-CoA pathway are positioned (in order of catalysis) proximally to ATP citrate lyase and acetyl-CoA acetyltransferase at the N-terminus of the primary scaffold. Scaffold binding sites for each enzyme in the upper cannabinoid pathway are positioned proximally to (immediately downstream of) the binding sites for the hexanoyl-CoA pathway enzymes. The scaffold binding sites for each enzyme in the mevalonate (or MEP) pathway are positioned (in order of catalysis) proximally to ATP citrate lyase and acetyl-CoA acetyltransferase at the C-terminus of the primary scaffold. The enzyme catalyzing the rate-limiting/committed step in cannabinoid biosynthesis (CBGA synthase, the final enzymatic step in the upper cannabinoid pathway) is located at the intersection of the converging cannabinoid precursor pathways near the scaffold midpoint.

### Assessment of Cannabinoidergic Potential by Transient Transfection

Competent yeast and/or green algae cells are transiently transfected with plasmids encoding various permutations of the scaffold and enzymes. To establish baseline cannabinoidergic capacity, cells first undergo transient transfection with the enzymes required for cannabinoid biosynthesis (but not the scaffolds), and biosynthesized cannabinoids are extracted, isolated, and quantified as described below (see “Cannabinoid Extraction, Isolation, and Analytical Characterization”). To measure the improvement in cannabinoidergic capacity conferred by multi-enzymatic scaffolding, a subset of the aforementioned cells is co-transfected with plasmids encoding one or more of the multi-enzymatic scaffolds described herein, and biosynthesized cannabinoids are extracted, isolated, and quantified. The presence of the plasmid DNA is confirmed by PCR, functional gene expression is confirmed by qRT-PCR, protein/polypeptide production is confirmed by Western blotting, and scaffolding of each enzyme is confirmed by co-immunoprecipitation of C-terminal myc/flag-tagged scaffolds followed by Western blot analysis of each co-immunoprecipitated enzyme.

### Engineering of Stable Cannabinoidergic Cell Lines

The constructs can be integrated into the genome of host cells such yeast, green algae, or other suitable hosts via stable transfection. Gene integration is confirmed by PCR, functional gene expression is confirmed by qRT-PCR, and protein/polypeptide production is confirmed by Western blotting. Gene expression/protein synthesis is confirmed by comparing both qRT-PCR and Western blot results among samples with and without genetic engineering. To assess the improvement in cannabinoidergic capacity conferred by multi-enzymatic scaffolding for stably engineered cannabinoidergic cell lines, cannabinoid biosynthesis will be compared among cells that are stimulated for enzyme but not scaffold expression and cells that are stimulated for enzyme and scaffold expression.

### Validation of Multi-Enzymatic Scaffolding

To verify successful multi-enzymatic scaffolding in both transiently transfected and stably engineered cells, a myc-tag (or other immunoprecipitable tag) is inserted at the N-terminal or C-terminal of the polypeptide scaffold(s). Scaffolded enzymes are selectively co-immunoprecipitated by affinity chromatography using anti-myc affinity beads. Western blots are performed to detect and quantify each co-immunoprecipitated enzyme.

### Aerobic Fed-Batch Fermentation

Stably engineered cannabinoidergic yeast, green algae, or other host cells are grown in bioreactors (or any other vessel) via aerobic batch fermentation (or any other culture technique).

### Cannabinoid Extraction, Isolation, and Analytical Characterization

Following sufficient elicitation of cannabinoid biosynthesis, engineered yeast/green algae cells are pelleted by centrifugation and washed with TBS. The supernatant (liquid culture media) is decanted and collected. Following washing with TBS, pelleted cells are resuspended in NaOH adjusted ethanol and lysed by iterative freeze-thawing and ultrasonication. Biosynthesized cannabinoid fermentates are then harvested from both lysates and supernatants via triplicate nonpolar extractions using hexane followed by ethyl-acetate. The resulting organic fractions are pooled and roto-evaporated. High-performance liquid chromatography with a diode array detector (HPLC-DAD) or gas chromatography-flame ionization detection (GC-FID) is then applied for quantitative and qualitative measurement of biosynthesized cannabinoids.

In the following examples, each 48-hour culture was lysed/homogenized by ultrasonication. Ultrasonicated samples were then subjected to triplicate liquid-liquid extractions with ethyl acetate (one volumetric equivalent of ethyl acetate per extraction). Following separation, the ethyl acetate fractions collected from each sample were pooled, and the pooled samples were centrifugally filtered. Ethyl acetate was then removed from each sample in a vacuum oven, and the residual samples were resuspended in 10 mL methanol for analytical characterization. Analytical characterization of all samples was conducted by a licensed, independent, third-party analytical testing facility (Precision Plant Molecules, Denver, CO). HPLC-DAD was utilized for quantitative and qualitative measurement of each parent and derivative cannabinoid as well as the cannabinoid precursor OVA.

### Example 2—Synthetic Gene Cassette Assembly/Synthesis, Plasmid Preparation, and Polycistronic Vector Construction

Five synthetic gene cassettes (entitled HCA, GPP, CAN, SCF, and SOL) were constructed for biosynthesizing cannabinoids in heterologous cells or acellular reaction buffers. See, FIG. 2B. The cassettes collectively encode all scaffold-binding engineered enzymes and the polypeptide scaffolds to which the engineered enzymes can bind.

The HCA gene cassette encoded scaffold-binding engineered enzymes for scaffolded hexanoyl-CoA biosynthesis, namely ACL, atoB, BHBD, ECH, ECR, and bktB, and encoded a soluble HCS for additional hexanoyl-CoA production from hexanoate-supplemented culture media or acellular reaction buffer. See, FIG. 13A. The GPP gene cassette encoded scaffold-binding engineered enzymes for scaffolded geranyl pyrophosphate (GPP) biosynthesis, namely HMGS, tHMGR, ERG12, ERG8, MVD1, IDI1, and ERG20^{WW}. See, FIG. 13B. The CAN gene cassette encoded scaffold-binding engineered enzymes for scaffolded OAC, malonyl-CoA, and CBGA biosynthesis, namely OS and OAC, ACC, and CBGAS, respectively, as well all enzymes for soluble (non-scaffolded) CBDA and CBCA biosynthesis, namely CBDAS and CBCAS, respectively. See, FIG. 13C. The SCF gene cassette encoded the polypeptide scaffolds for bidirectional scaffolded cannabinoid biosynthesis and scaffolded malonyl-CoA biosynthesis, namely the cannabinoidergic metabolon scaffold (CBSCF) and the malonyl-CoA metabolon scaffold (MCASCF), respectively, as well as additional copies of both ACL and atoB to enhance acetyl-CoA biosynthesis from supplemental and/or endogenous citrate and acetoacetyl-CoA biosynthesis from acetyl-CoA, respectively. See, FIG. 13D. The SOL gene cassette lacked the polypeptide scaffolds for bidirectional scaffolded cannabinoid biosynthesis and scaffolded malonyl-CoA biosynthesis (i.e., it was used for soluble cannabinoid biosynthesis) but, analogous to the SCF gene cassette, encoded additional copies of ACL and atoB to enhance acetyl-CoA biosynthesis from supplemental and/or endogenous citrate and acetoacetyl-CoA biosynthesis from acetyl-CoA. See FIG. 13A for the amino acids sequences of the ACL and atoB engineered enzymes.

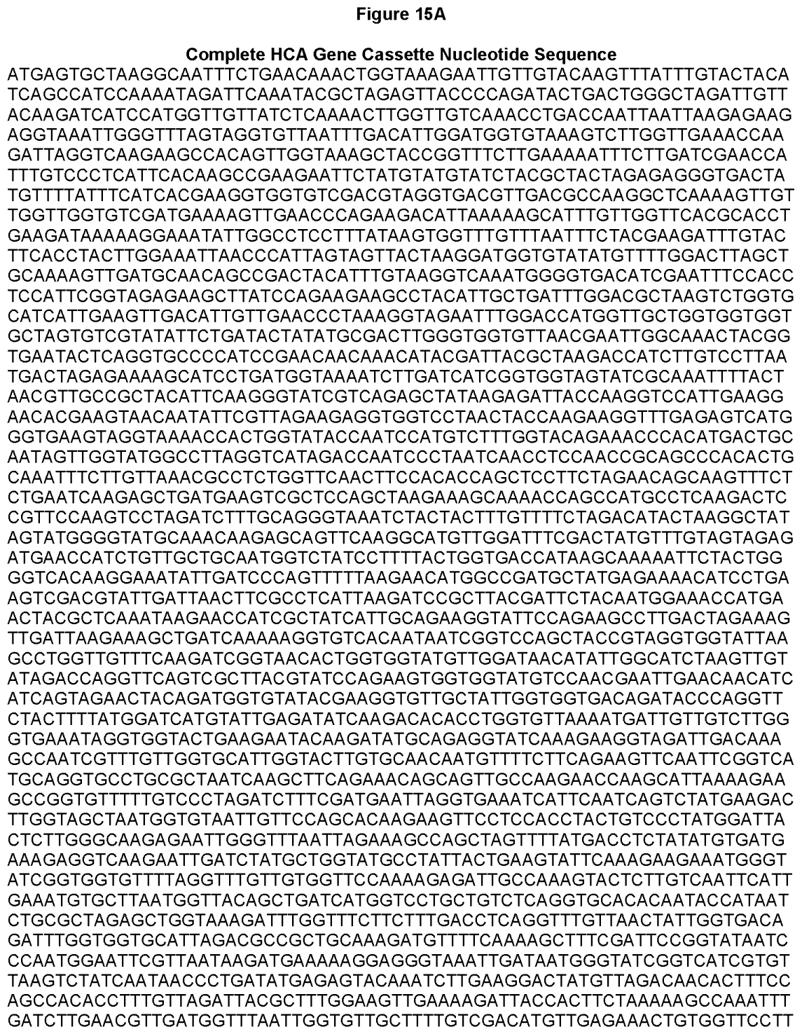

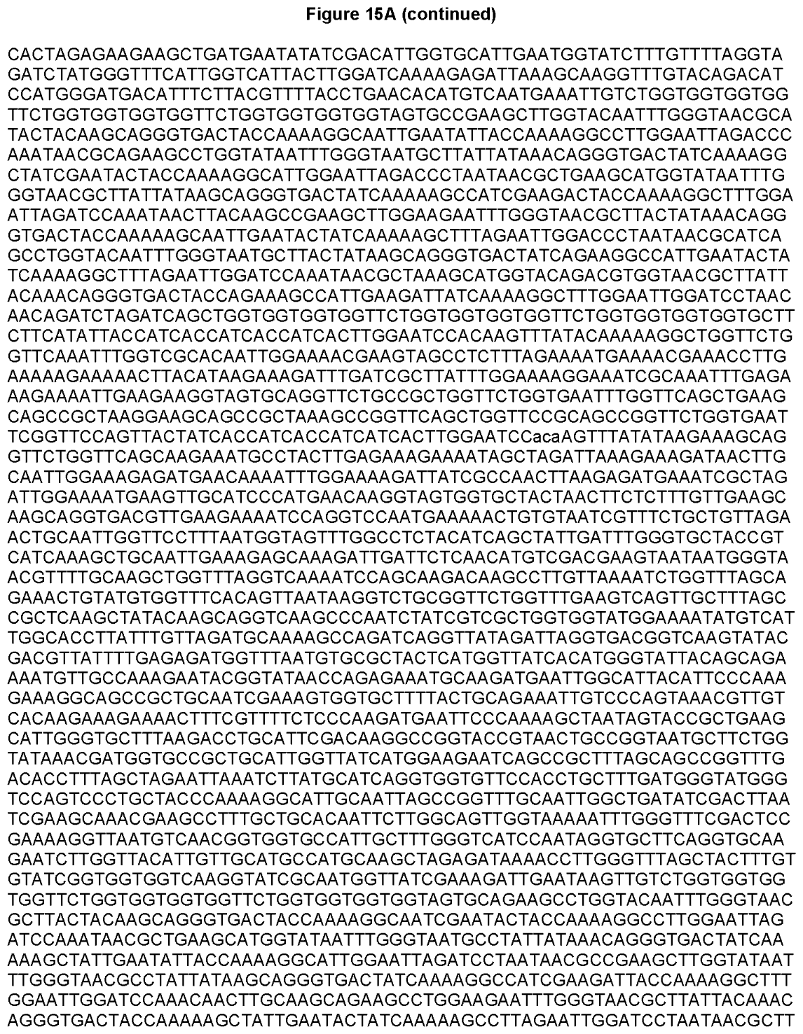

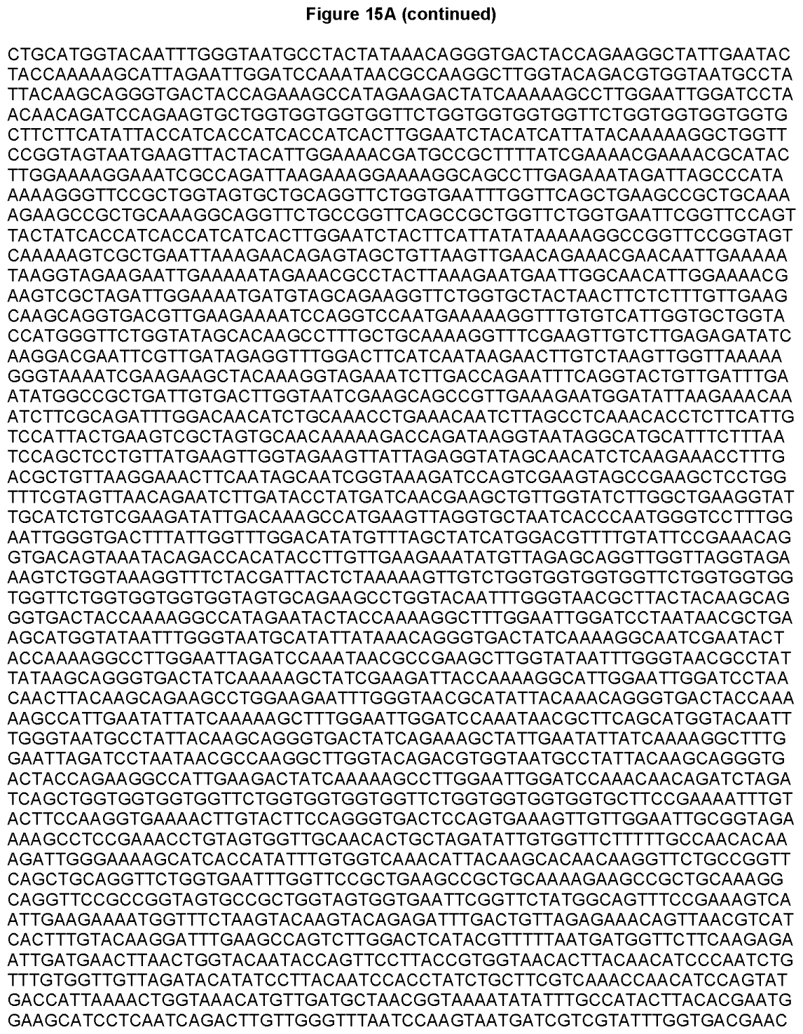

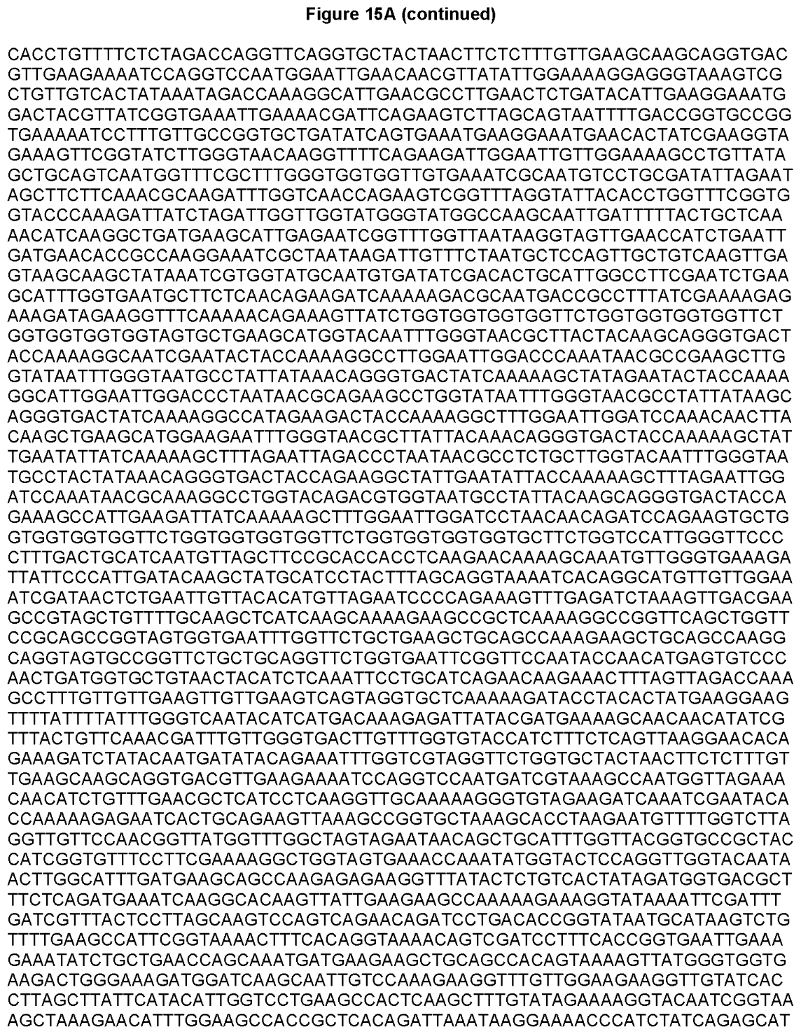

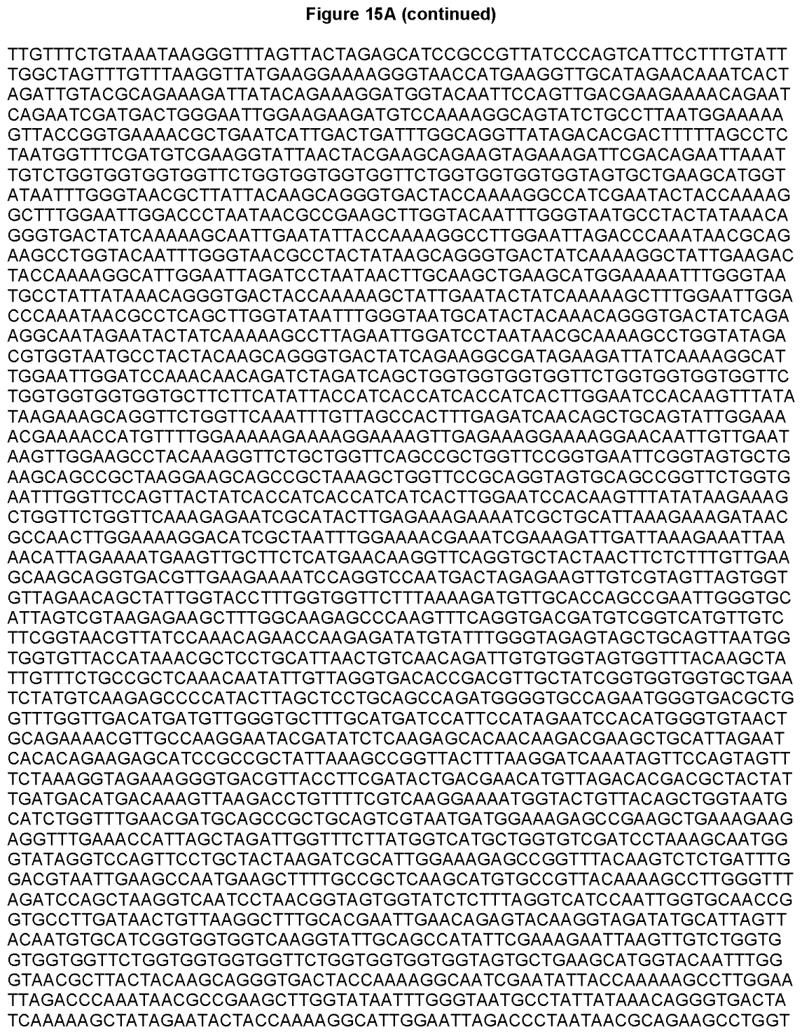

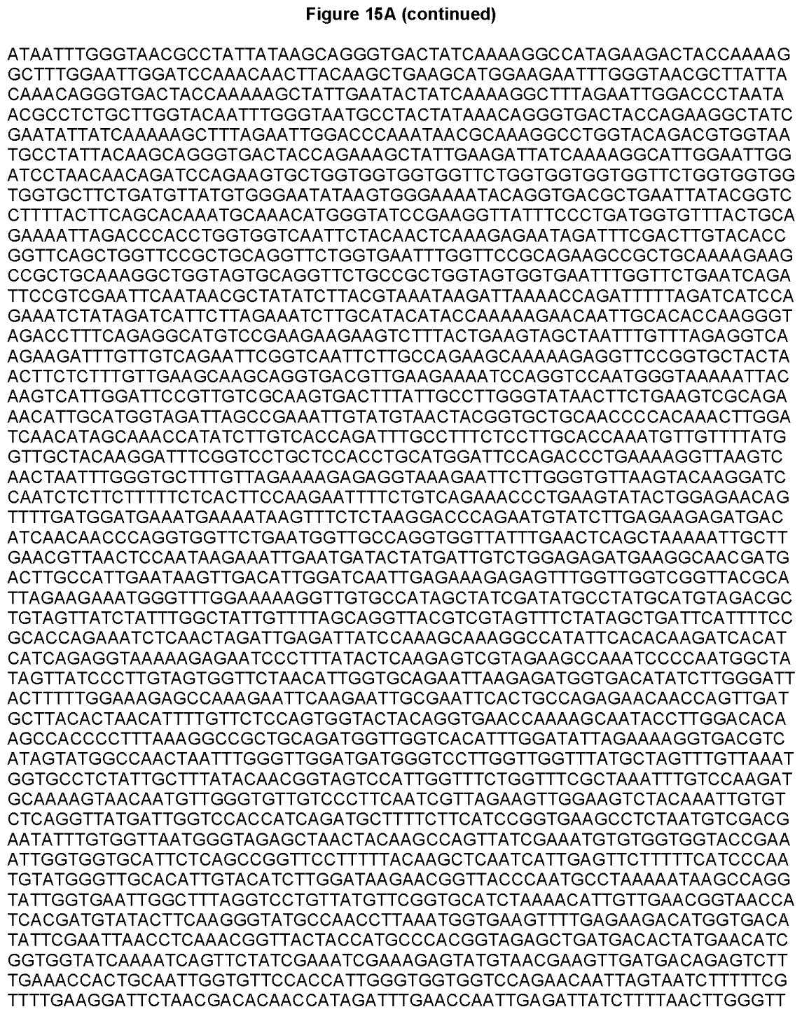

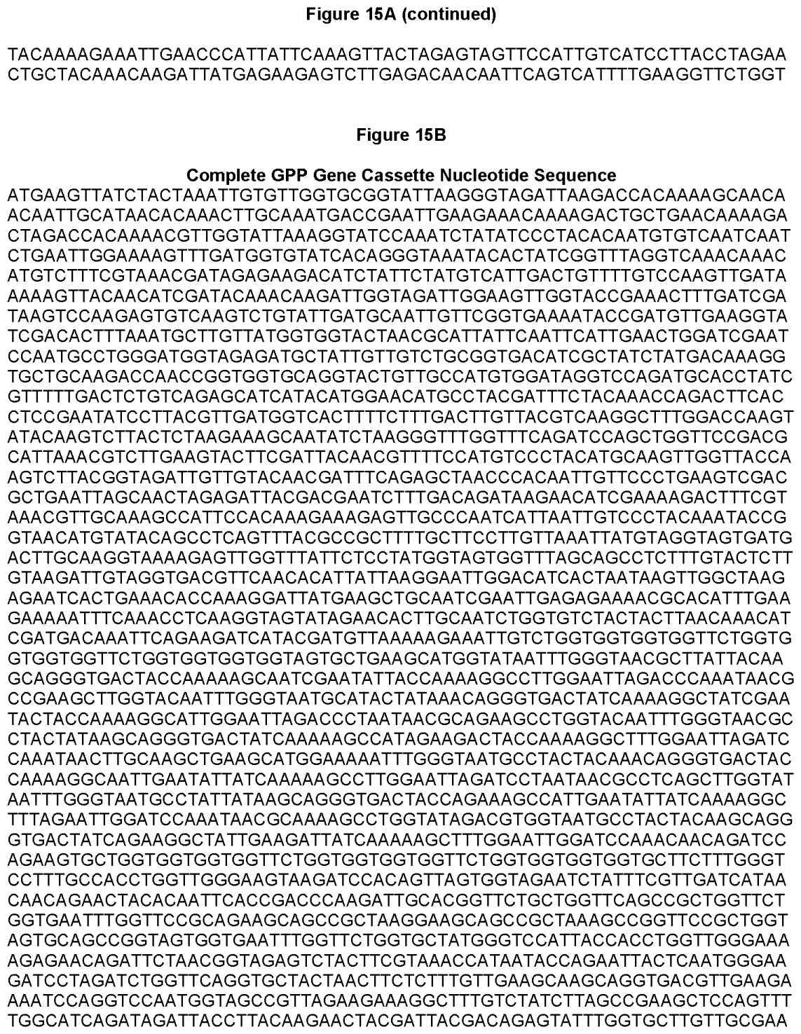

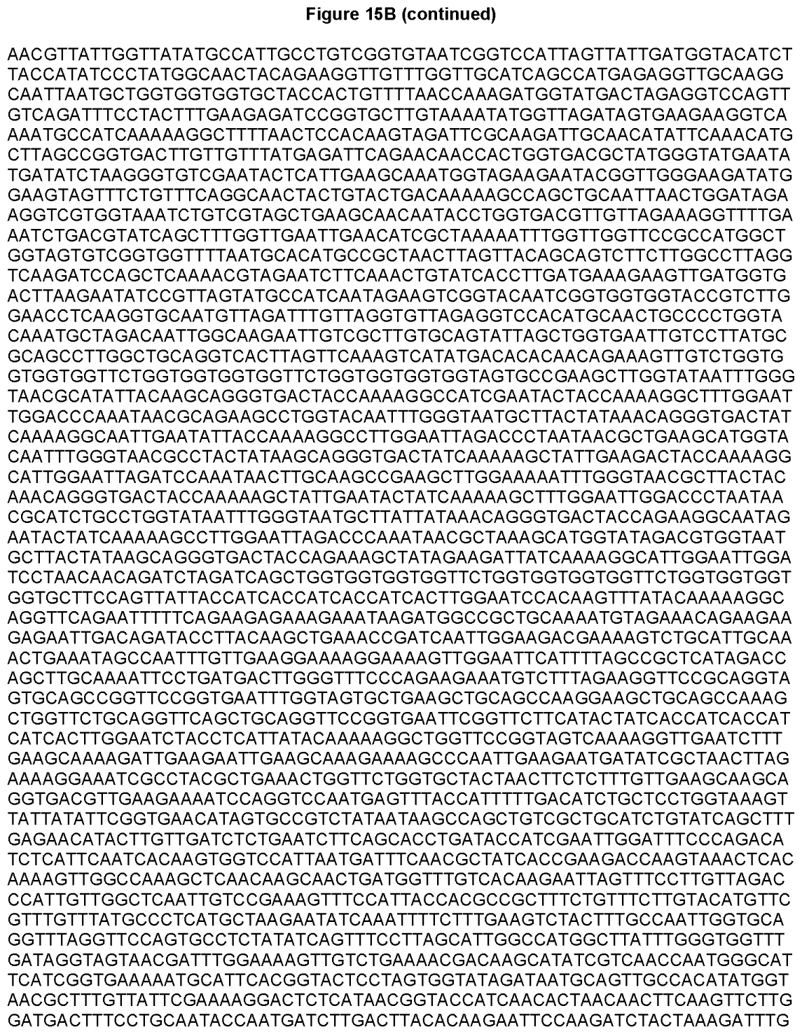

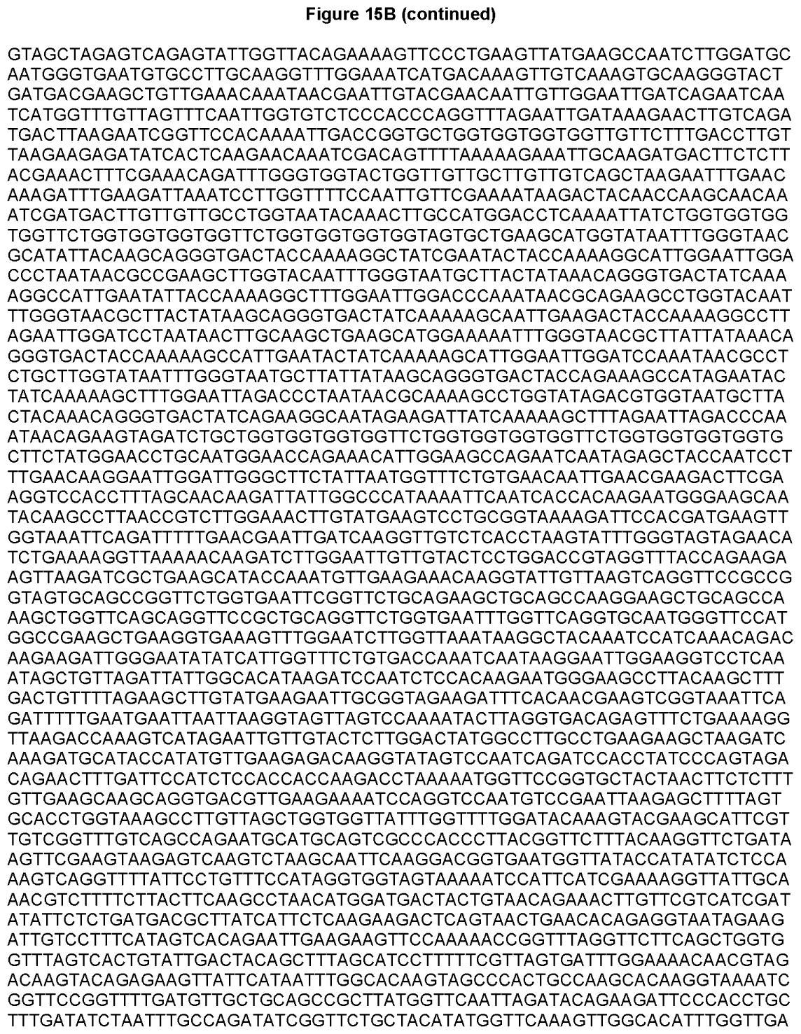

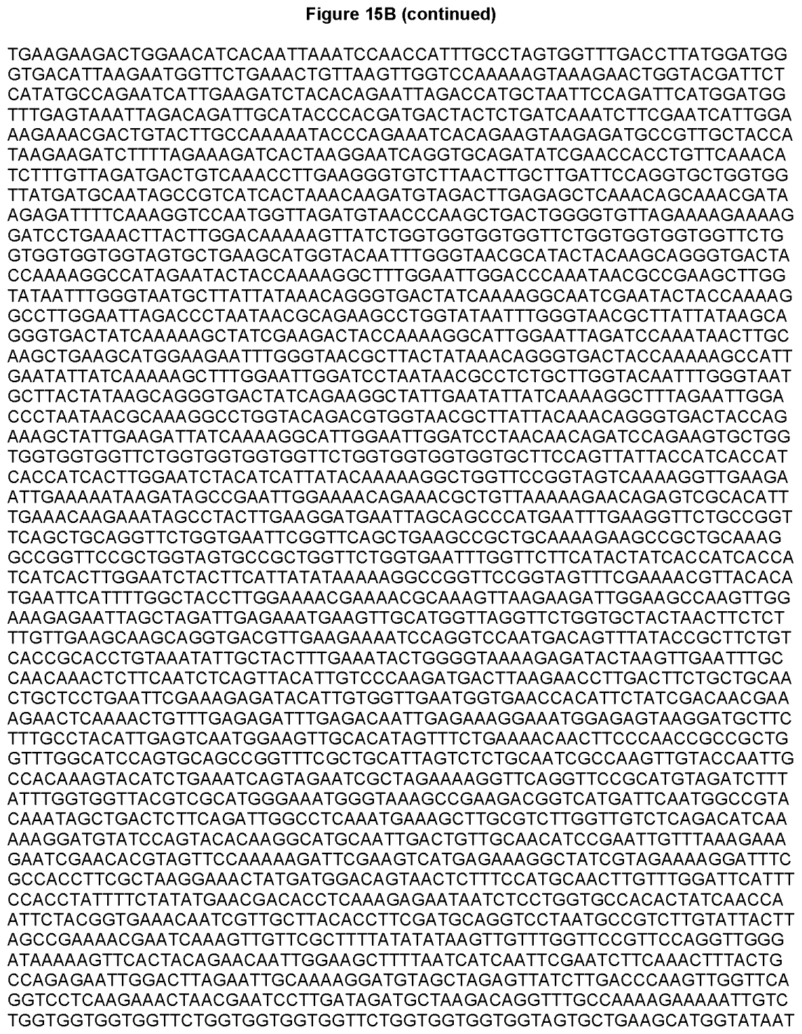

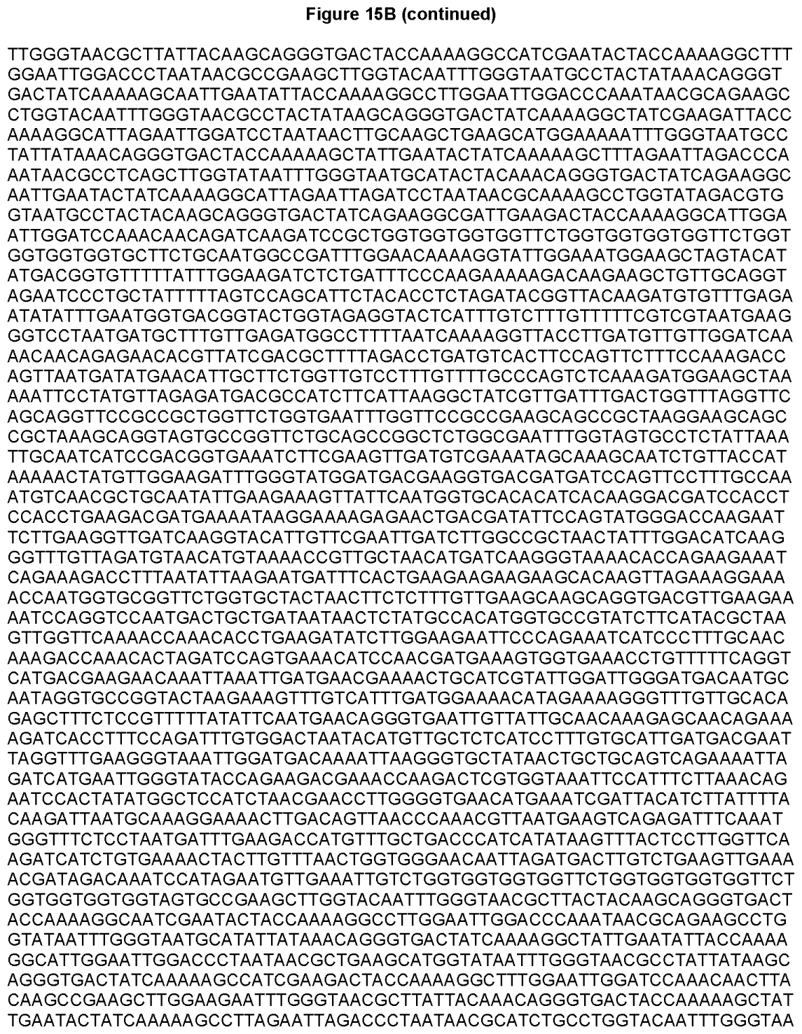

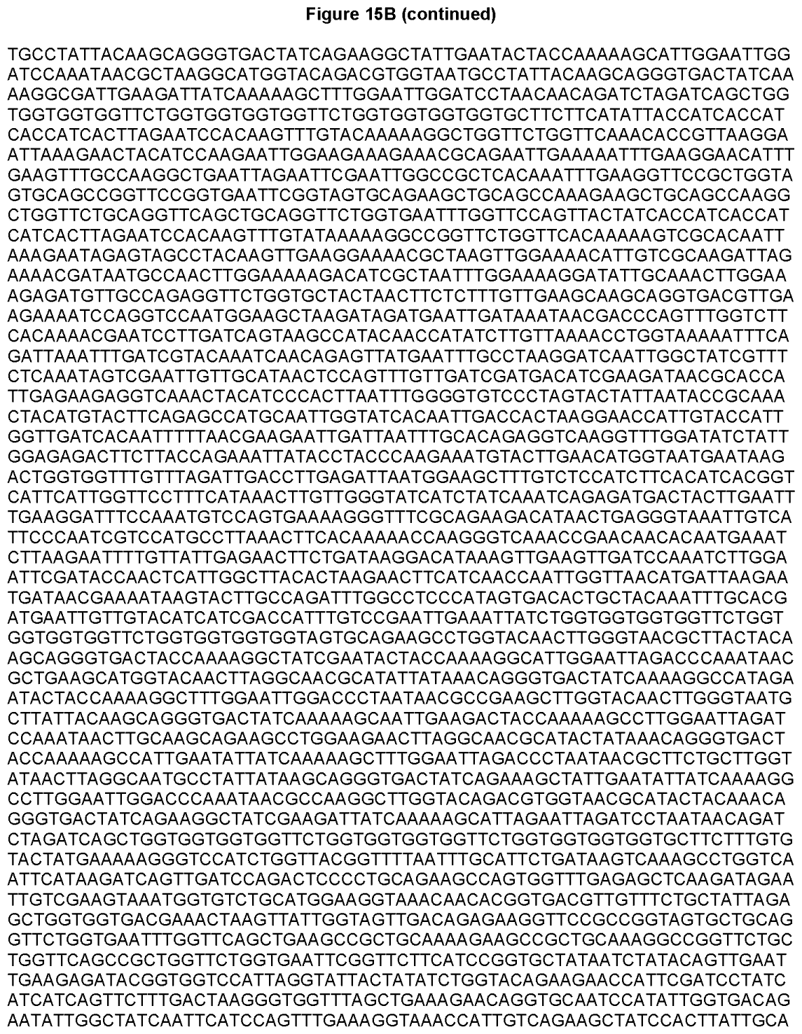

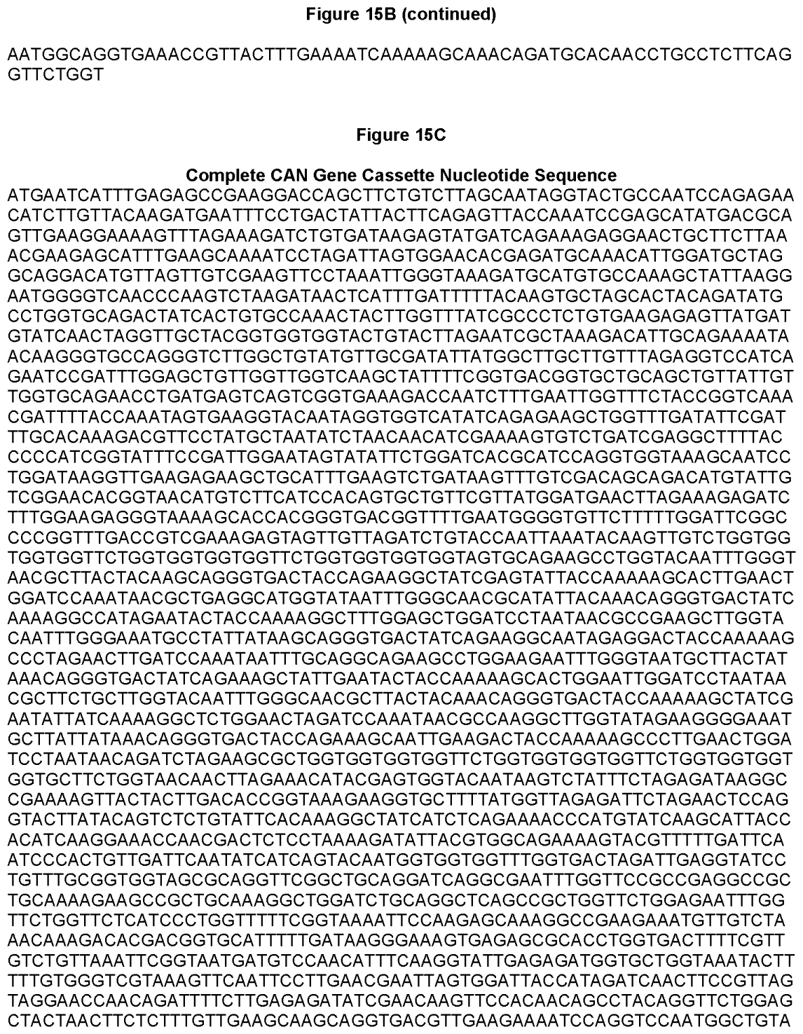

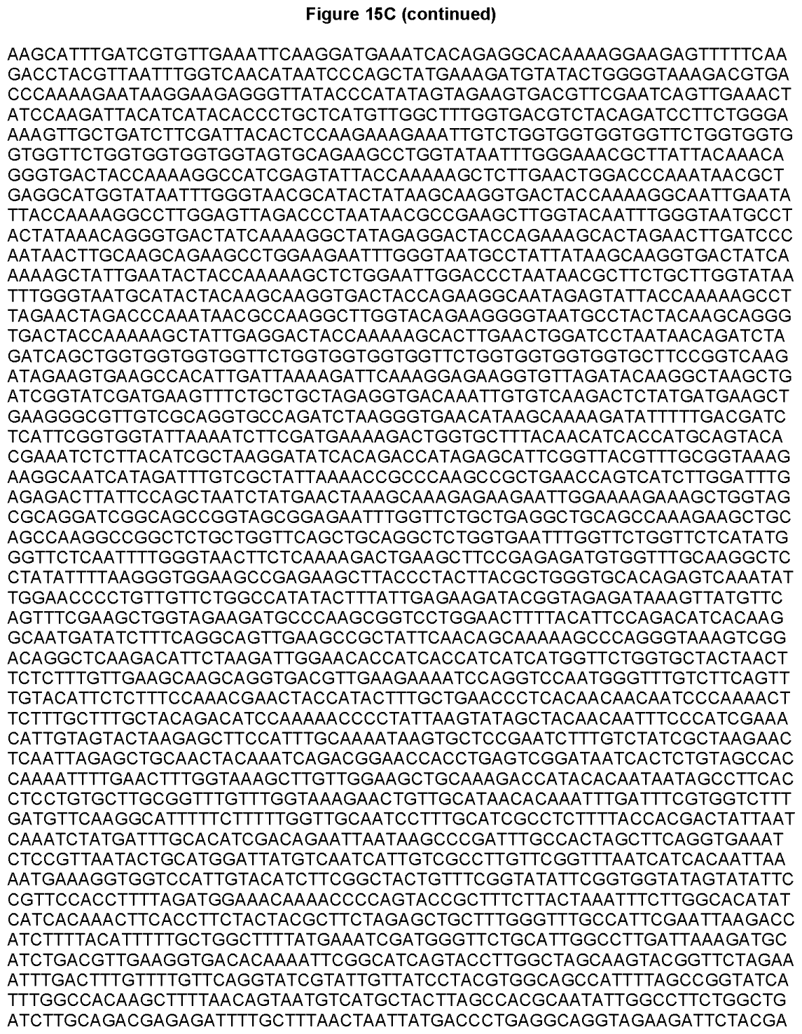

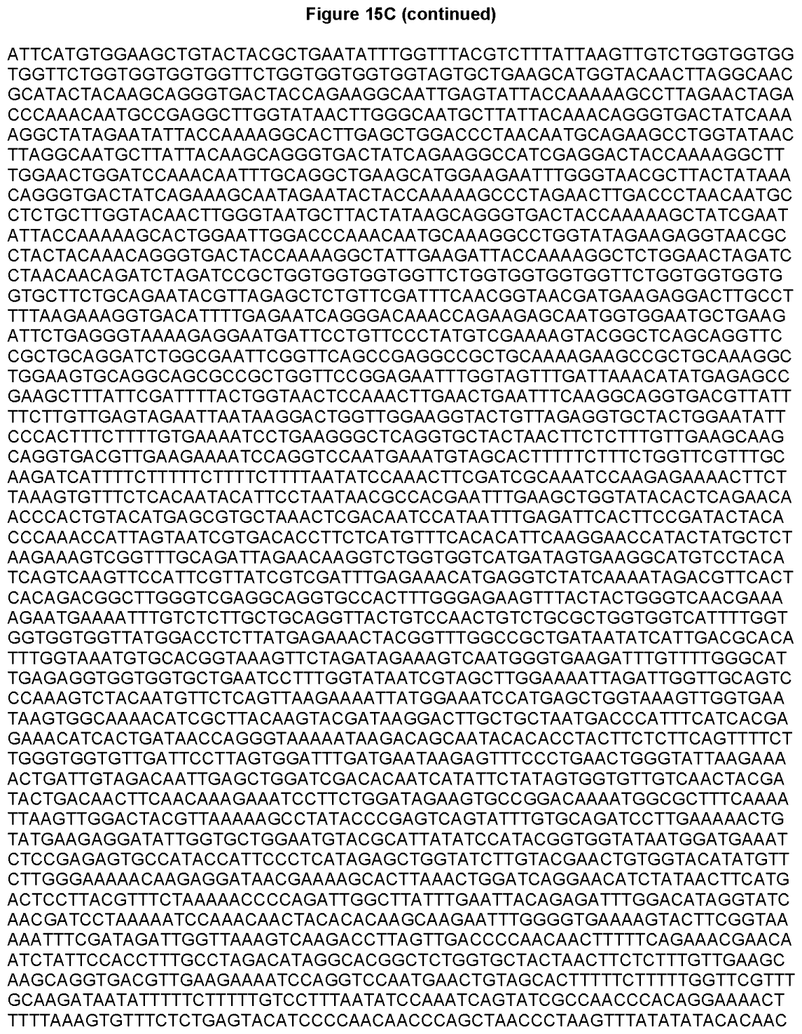

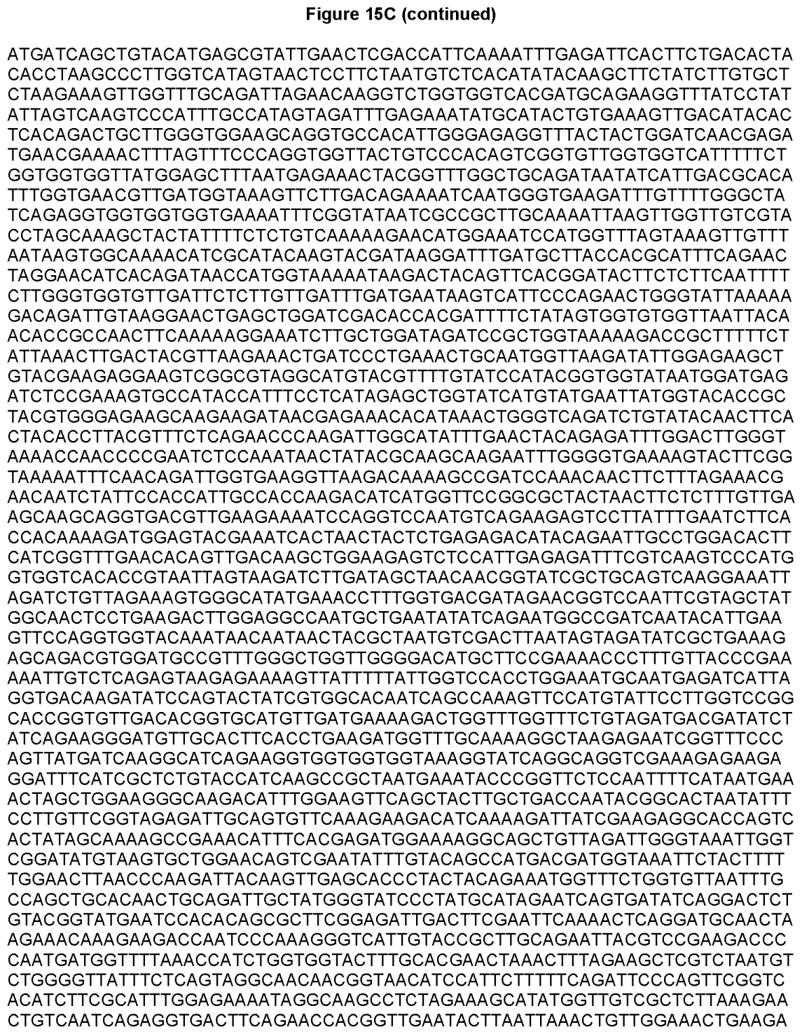

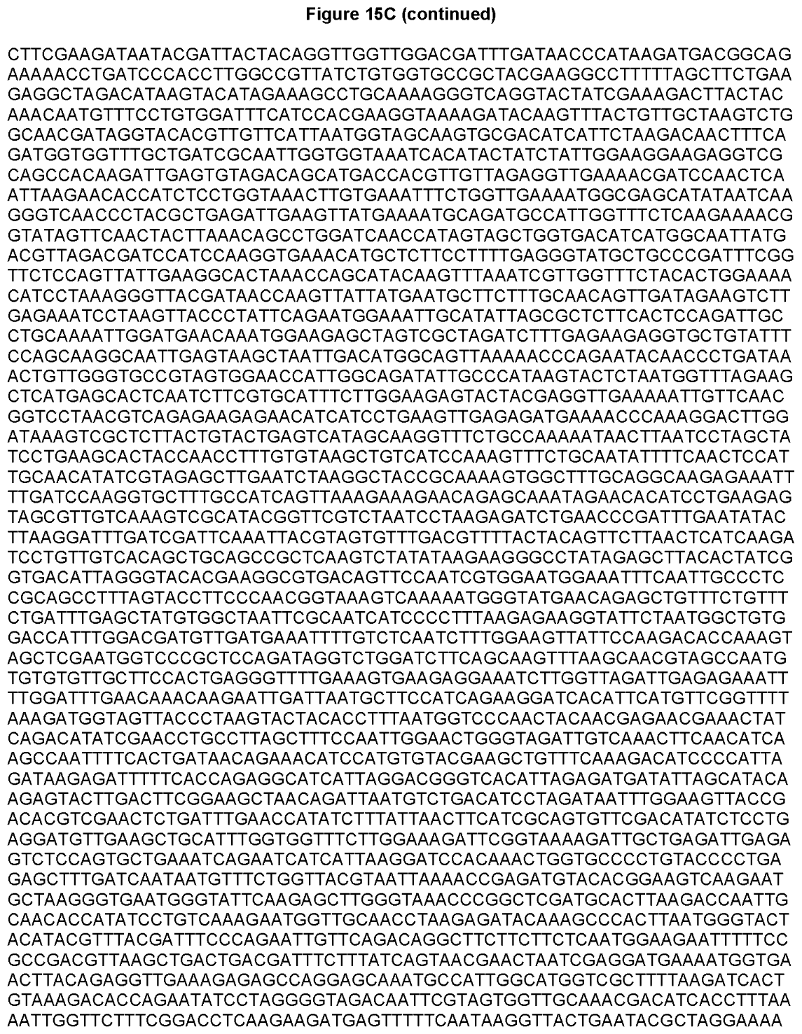

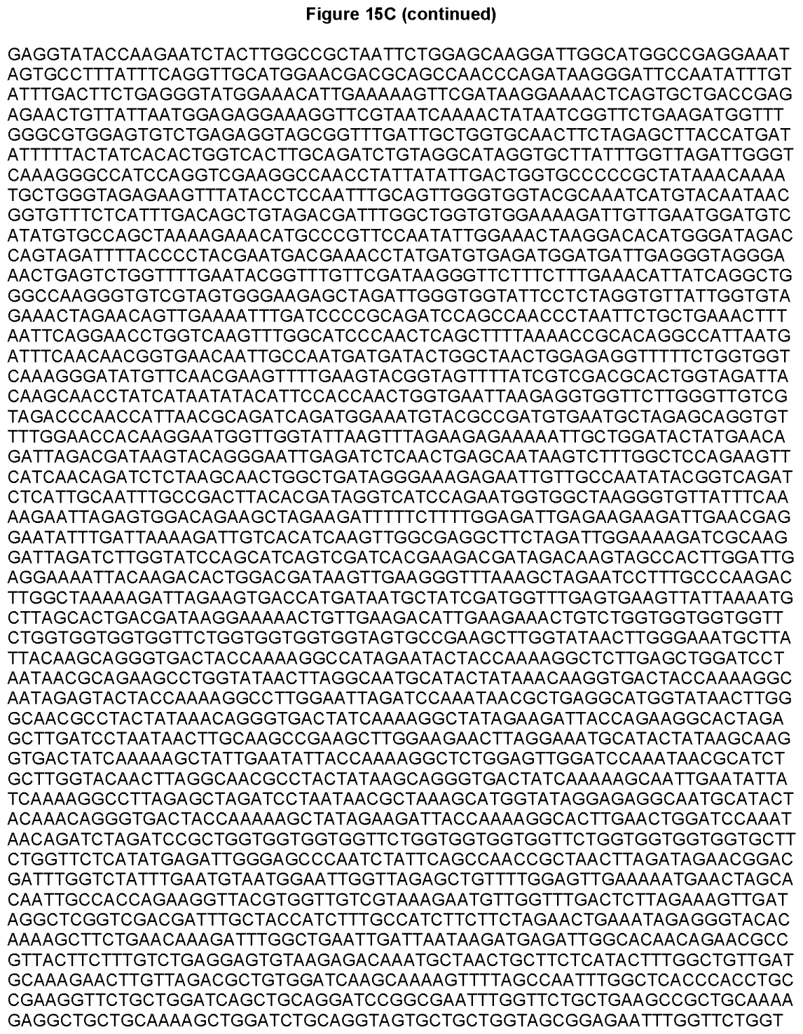

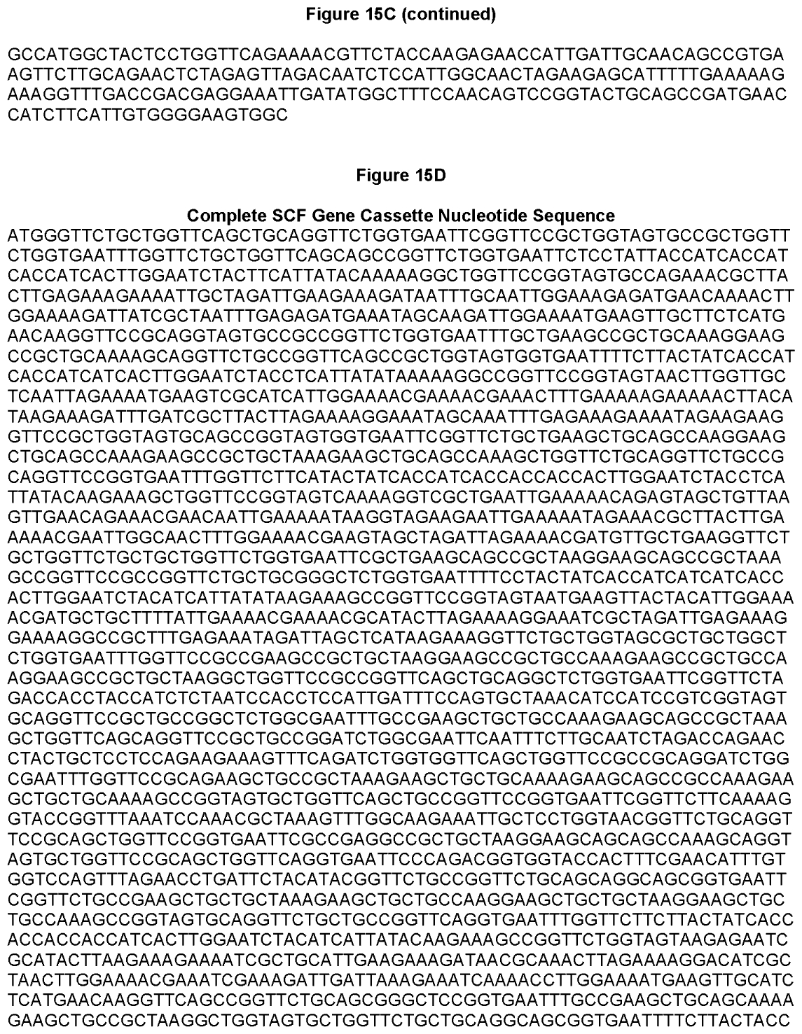

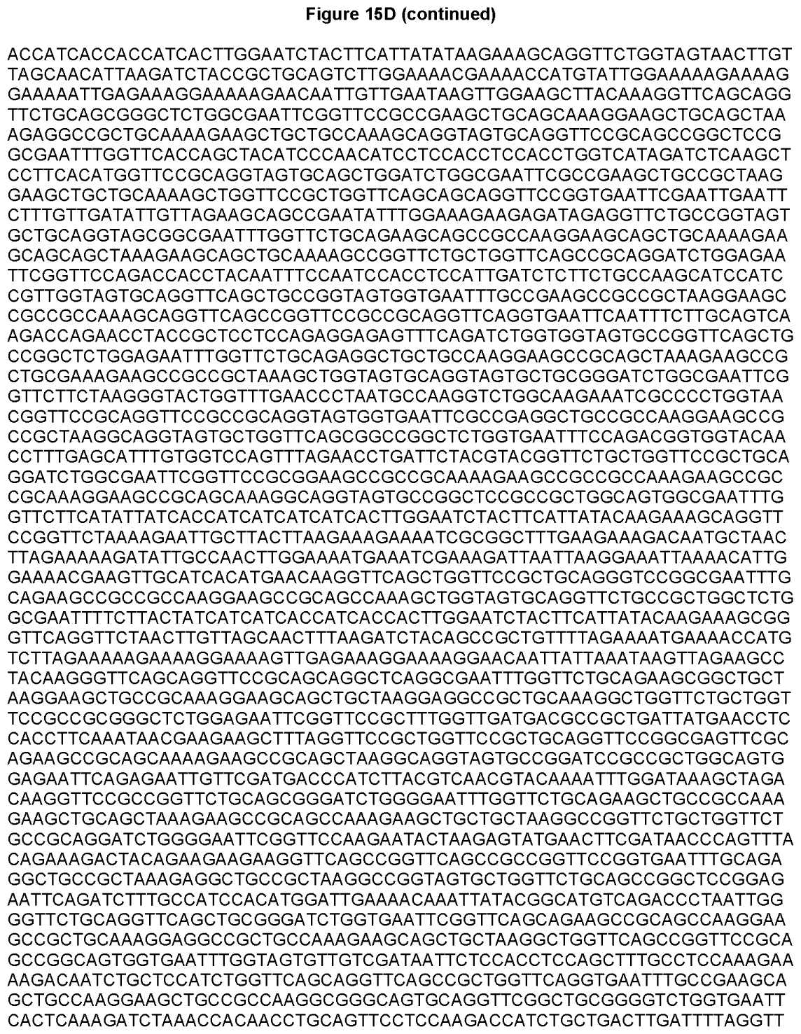

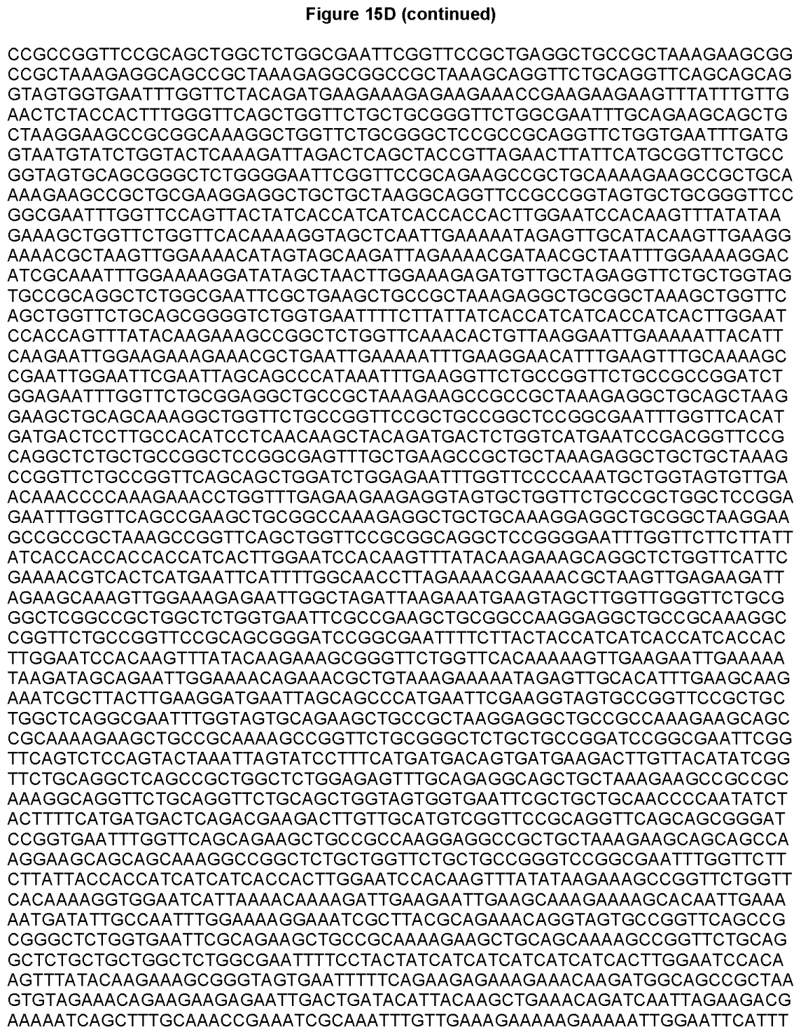

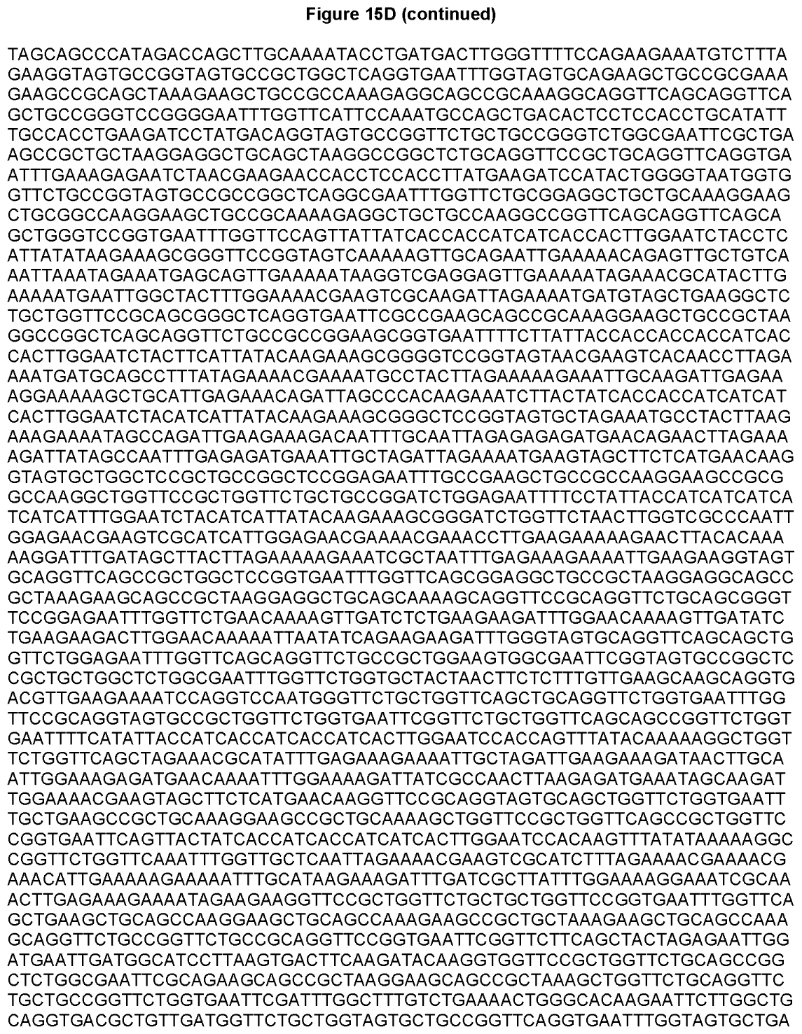

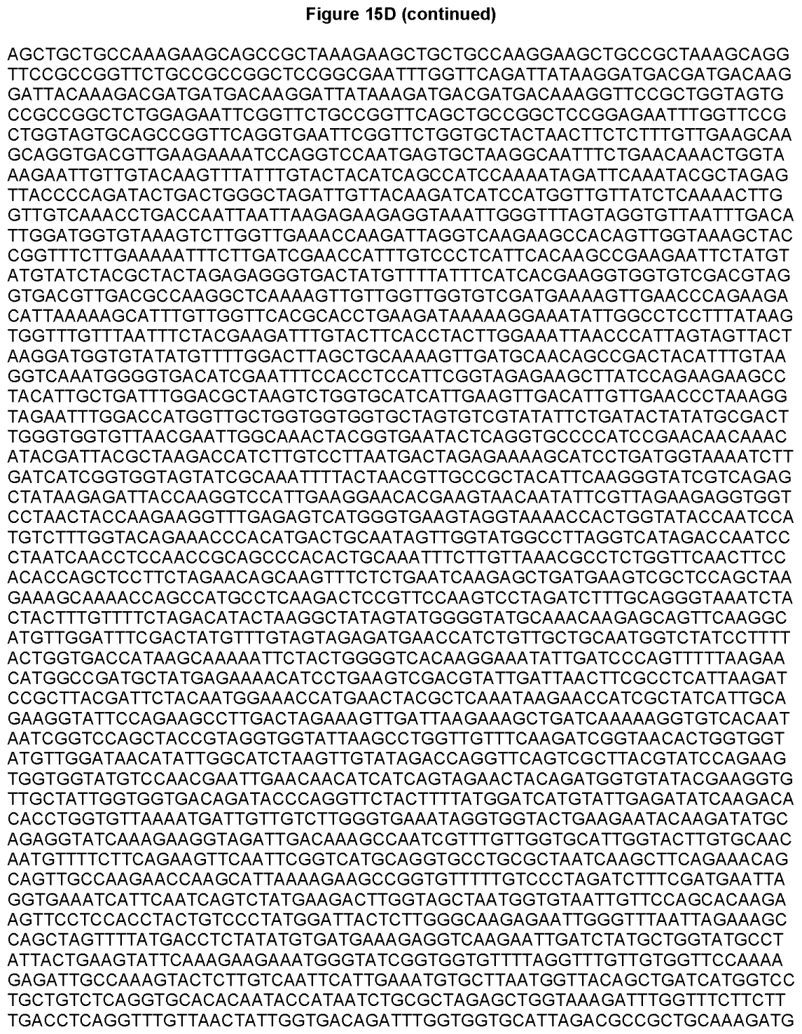

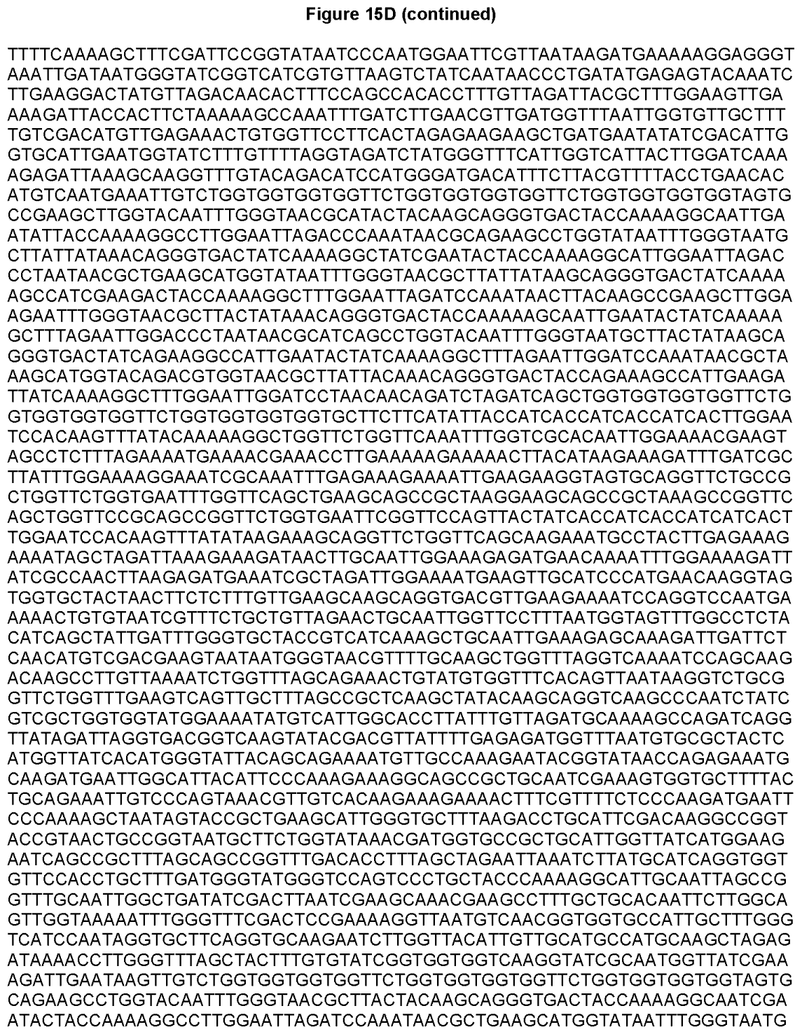

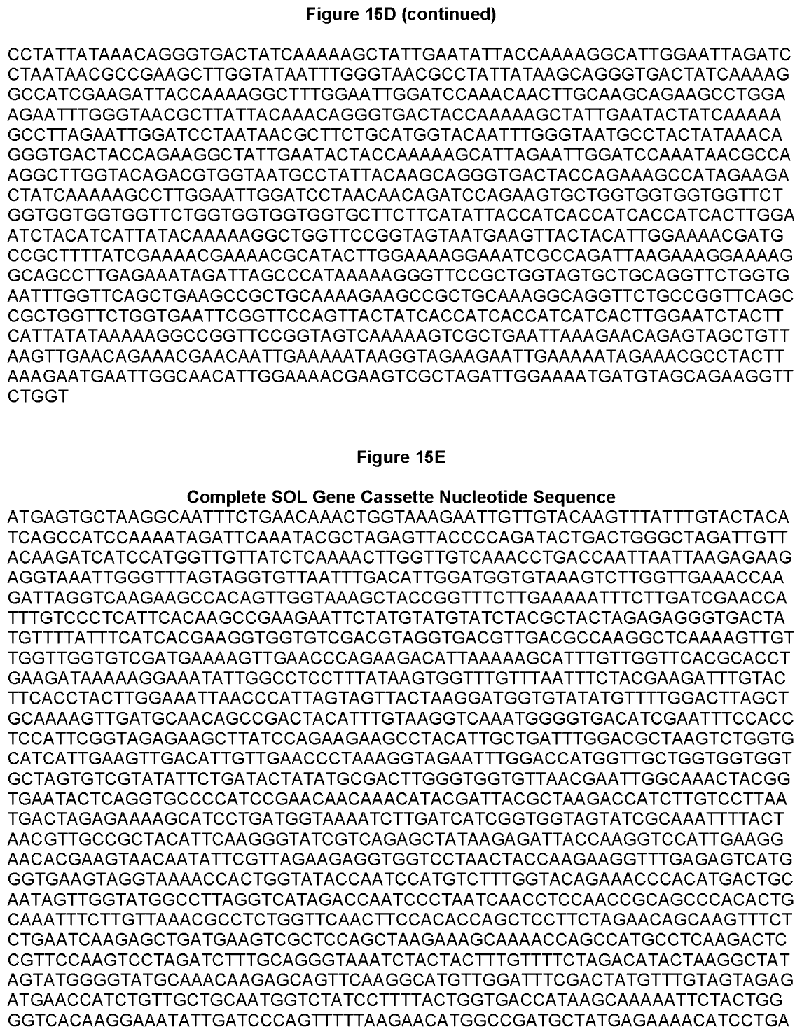

Gene cassettes were assembled/synthesized using self-cleaving 2A peptides (P2As) to link multiple codon-optimized (for S. cerevisiae) gene sequences assigned to each cassette. To improve P2A cleavage, a GSG linker (comprised of a single serine residue flanked by single glycine residues) was inserted at the interface between each constituent gene sequence and the P2A linker sequence to which it was fused (of the format: gene cassette sequence 1—SG—P2A linker—gene cassette sequence 2—GSG—P2A linker—gene cassette sequence 3—GSG—P2A linker—) and so forth. See, FIGS. 14A-14D for codon-optimized nucleic acid sequences encoding the engineered enzymes and scaffolds. Following assembly, each synthetic gene cassette was inserted into a pCCI-Brick plasmid, resulting in plasmids entitled pHCA, pGPP, pCAN, pSCF, and pSOL as described in Table 3. See, FIGS. 15A-15E for the complete gene cassette inserted into the plasmids. Each of these plasmids then were used to amplify each synthetic gene cassette via standard plasmid prep. Plasmid DNA encoding each complete synthetic gene cassette was cloned into the SpeI/XhoI cloning site of polycistronic yeast auxotrophic selection vectors, resulting in vectors entitled vHCA, vGPP, vCAN, vSCF, and vSOL as described in Table 3, to allow iterative antibiotic/auxotrophic selection of only those cells that were transformants of one or more such polycistronic vector(s).

TABLE 3
HCA Gene Cassette

| | Cassette | pCCI-Brick #1 | | Yeast Vector |
|---|---|---|---|---|
| Gene ID | Position | ID | Yeast Vector | ID |
| ACL | 1 | pHCA | pESC-TRP | vHCA |
| atoB | 2 | | | |
| BHBD | 3 | | | |
| ECH | 4 | | | |
| ECR | 5 | | | |
| bktB | 6 | | | |
| HCS | 7 | | | |

MVA Gene Cassette

| | Cassette | pCCI-Brick #2 | | Yeast Vector |
|---|---|---|---|---|
| Gene ID | Position | ID | Yeast Vector | ID |
| HMGS | 1 | pGPP | pESC-LEU | vGPP |
| tHMGR | 2 | | | |
| ERG12 | 3 | | | |
| ERG8 | 4 | | | |
| MVD1 | 5 | | | |
| IDI1 | 6 | | | |
| ERG20^{WW} | 7 | | | |

CAN Gene Cassette

| | Cassette | pCCI-Brick #3 | | Yeast Vector |
|---|---|---|---|---|
| Gene ID | Position | ID | Yeast Vector | ID |
| OS | 1 | pCAN | pESC-HIS | vCAN |
| OAC | 2 | | | |
| CBGAS | 3 | | | |
| CBDAS | 4 | | | |
| CBCAS | 5 | | | |
| ACC | 6 | | | |

SCFLD Gene Cassette

| | Cassette | pCCI-Brick #4 | | Yeast Vector |
|---|---|---|---|---|
| Gene ID | Position | ID | Yeast Vector | ID |
| CBSCF | 1 | pSCF | pESC-URA #1 | vSCF |
| MCASCF | 2 | | | |
| ACL | 3 | | | |
| atoB | 4 | | | |

NSCFLD Gene Cassette

| | Cassette | pCCI-Brick #5 | | Yeast Vector |
|---|---|---|---|---|
| Gene ID | Position | ID | Yeast Vector | ID |
| ACL | 1 | pSOL | pESC-URA #2 | vSOL |
| atoB | 2 | | | |

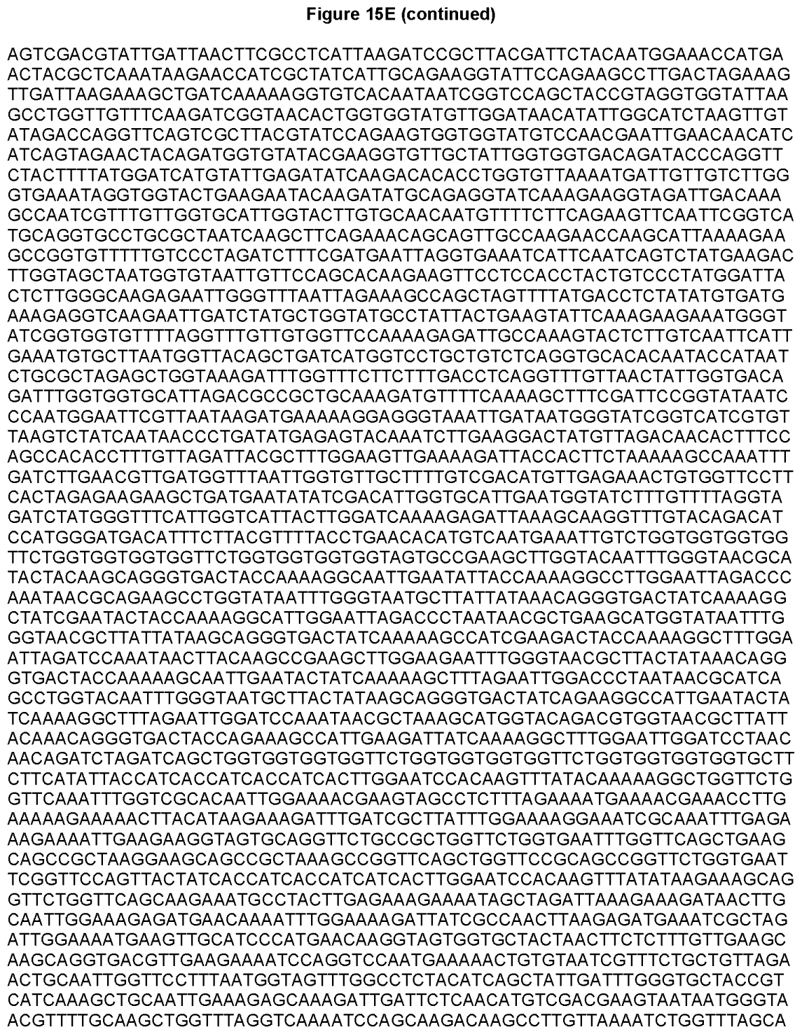

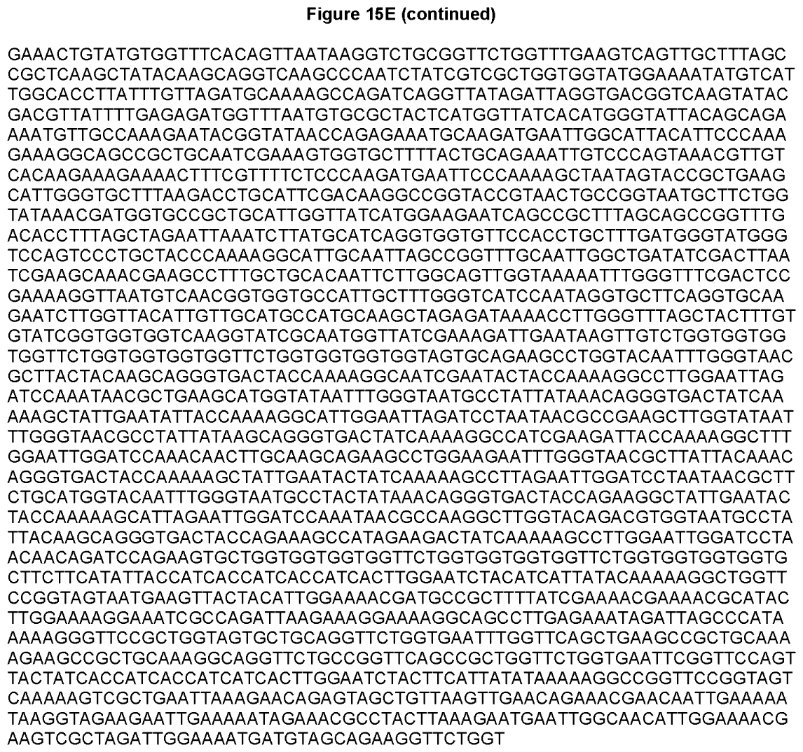

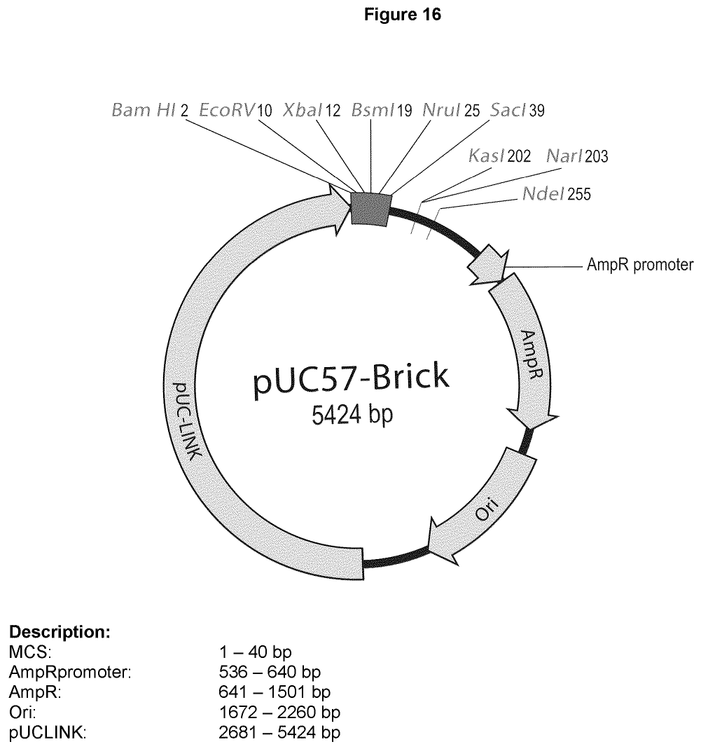

The genes assigned to each synthetic gene cassette as well as the plasmids and vectors into which each synthetic gene cassette was inserted are listed in Table 3, the amino acid sequences encoded by each synthetic gene cassette are provided in FIGS. 13A-13D, the codon-optimized nucleotide sequence fragments comprising each synthetic gene cassette are detailed in FIGS. 14A-14D, the complete nucleotide sequences of each fully-assembled synthetic gene cassette (the complete insert sequences for each plasmid and expression vector) are provided in FIGS. 15A-15E, a general map of pCCI-Brick plasmids is shown in FIG. 16, and a general map of a polycistronic yeast auxotrophic selection vector is shown in FIG. 17.

### Example 3—Engineering of Cannabinoidergic Cells

To engineer a novel heterologous pathway for the biosynthesis of cannabinoids from citrate, and to evaluate the impacts of bidirectional multi-enzymatic scaffolding thereon, competent S. cerevisiae cells were sequentially/iteratively transformed with, and auxotrophically selected for, expression of vHCA, vGPP, vCAN, and either vSCF (for scaffolded cannabinoid biosynthesis) or vSOL (for non-scaffolded/soluble cannabinoid biosynthesis) constructs.

All vector transformation and auxotrophic selection procedures were conducted as follows. An aliquot of an overnight S. cerevisiae culture was inoculated into 100 mL YPD media (10 g/L yeast nitrogen base, 20 g/L peptone, and 20 g/L D-(+)-glucose) to OD₆₀₀ₙₘ=0.3 (stationary phase) and grown to OD₆₀₀ₙₘ=1.6 in an orbital shaker at 30° C. and 225 RPM. Cells then were harvested by centrifugation at 3000×g for 3 minutes followed by aspiration of media. The harvested cell pellet was next washed 2× with 50 mL chilled nuclease-free water and 1× with 50 mL chilled electroporation buffer (1M sorbitol/1 mM CaCl₂). Washed cells were conditioned by incubation for 30 minutes in 20 mL 0.1M LiAc/10 mM DTT in an orbital shaker at 30° C. and 225 RPM, harvested, washed 1× with 50 mL electroporation buffer, harvested, and resuspended in 100 μL electroporation buffer. The resuspended cells were transformed with a quantity of vector containing 3 μg of the target DNA insert (calculated using the vector-insert ratio for each vector) by electroporation at 2.5 kV and 25 μF. To the electroporated cell suspension was then added 8 mL of YPD media containing 1M sorbitol, and the resulting suspension was incubated for one hour in an orbital shaker at 30° C. and 225 RPM. To isolate target transformants by auxotrophic selection, cells were harvested, resuspended in the appropriate yeast nitrogen base (YNB) dropout (selection) media as subsequently described for each iterative transformation step, transferred to a baffled culture flask, and incubated overnight in an orbital shaker at 30° C. and 225 RPM. The transformation and selection protocols were utilized sequentially for each assigned vector.

Applying the aforementioned approach, an initial culture of electrocompetent S. cerevisiae cells was first transformed with vHCA, which encodes scaffold-binding engineered enzymes required for biosynthesis of HCA from citrate. Cells transformed with vHCA (designated yHCA) were selected for by resuspension and incubation in tryptophan-deficient YNB media. Selected yHCA cells (i.e., cells that grew in tryptophan-deficient YNB media) were next transformed with vGPP, which encodes scaffold-binding engineered enzymes required for biosynthesis of GPP from citrate. Cells co-transformed with vHCA and vGPP (designated yHCAGPP) were selected for by resuspension and incubation in tryptophan- and leucine-deficient YNB media. Selected yHCAGPP cells (i.e., cells that grew in tryptophan- and leucine-deficient YNB media) were then transformed with vCAN, which encodes scaffold-binding engineered enzymes required for biosynthesis of malonyl-CoA from citrate, olivetol from HCA and malonyl-CoA, OVA (olivetoic acid) from olivetol, and CBGA from OVA and GPP as well as soluble enzymes required for biosynthesis of CBDA and CBCA from CBGA). Cells co-transformed with vHCA, vGPP, and vCAN (designated yCB_{Parent}) were selected for by resuspension and incubation in tryptophan-, leucine-, and histidine-deficient YNB media.

The yCB_{Parent} culture containing cells that grew in tryptophan-, leucine-, and histidine-deficient YNB media then was split into two separate cultures. The first of the split yCB_{Parent} cultures was transformed with vSCF, which encodes CBSCF (cannabinoidergic metabolon scaffold) and MCASCF (malonyl-CoA metabolon scaffold) as well as additional copies of ACL and atoB. Cells co-transformed with vHCA, vGPP, vCAN, and vSCF (designated yCB_{SCF}) were selected for by resuspension and incubation in tryptophan-, leucine-, histidine-, and uracil-deficient YNB media. The second of the split yCB_{Parent} cultures was transformed with vSOL, which encodes additional copies of ACL and atoB but lacks both CBSCF and MCASCF. Cells co-transformed with vHCA, vGPP, vCAN, and vSOL (designated yCB_{SOL}) were also selected for by resuspension and incubation in tryptophan-, leucine-, histidine-, and uracil-deficient YNB media.

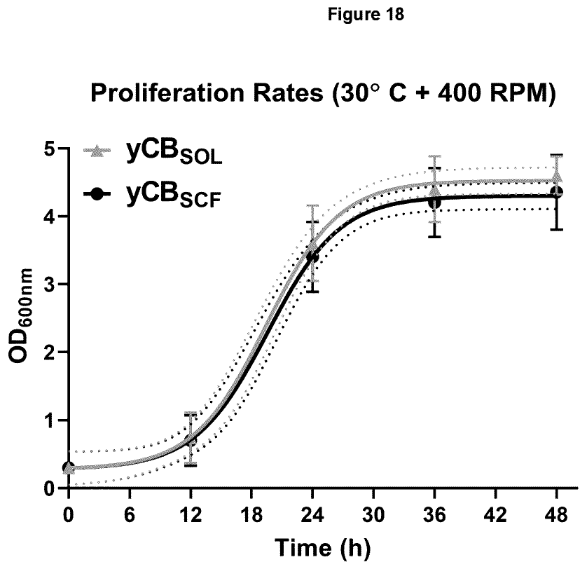

To quantify the improvement in cannabinoidergic capacity conferred by multi-enzymatic scaffolding, cannabinoid titers were compared between triplicate yCB_{SOL} and yCB_{SCF} cultures grown in 100 mL YPD media for 48 hours at 30° C. and 400 RPM in an incubator-shaker. To compare the proliferation rates of yCB_{SOL} and yCB_{SCF}, each culture was initially diluted to OD₆₀₀ₙₘ=0.3, and OD₆₀₀ₙₘ measurements were recorded in 12-hour intervals thereafter. Proliferation curves are depicted in FIG. 18. The extra sum-of-squares F-test indicated that the proliferation curves of yCBSCF and yCBSOL cultures did not significantly differ for any parameter over the 48-hour incubation period, indicating that scaffolding does not impact cellular proliferation.

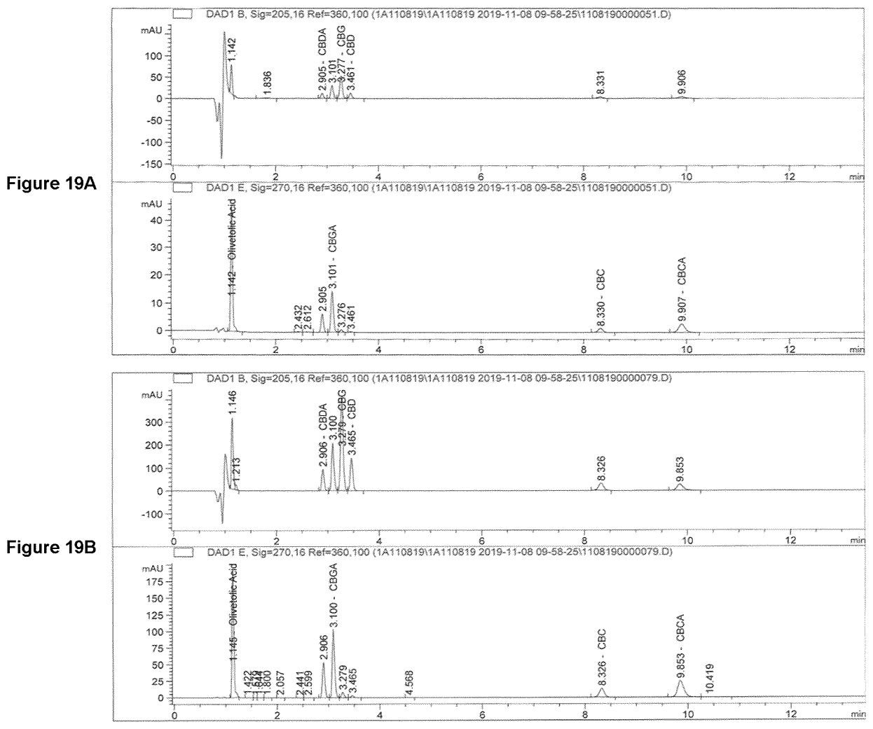

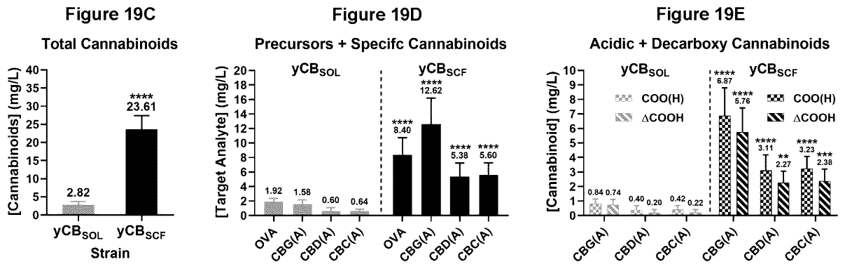

Total cannabinoid titers, parent (carboxylated) cannabinoid (CBGA, CBDA, and CBCA) titers, derivative (decarboxylated) cannabinoid (CBG, CBD, and CBC) titers, and cannabinoid precursor (OVA) titers were measured. As shown in FIGS. 19A-19E, mixed ANOVA detected main effects of strain (F_{1,4}=943.8; p<0.0001) and analyte (cannabinoid and cannabinoid precursor) titers (F_{10,40}=216.4; p<0.0001) and a significant strain x analyte interaction (F_{10,40}=131.4; p<0.0001). Relative to yCBSOL cultures, yCBSCF cultures exhibited increased total cannabinoid (p<0.0001), OVA precursor (p<0.0001), CBG(A) (p<0.0001), CBD(A) (p<0.0001), CBC (A) (p<0.0001), CBGA (p<0.0001), CBDA (p<0.0001), CBCA (p<0.0001), CBG (p<0.0001), CBD (p<0.01), and CBC (p<0.001) titers.

### Example 4—Impacts of Citrate and Hexanoate Supplementation on Scaffolded and Soluble Cannabinoid Biosynthesis

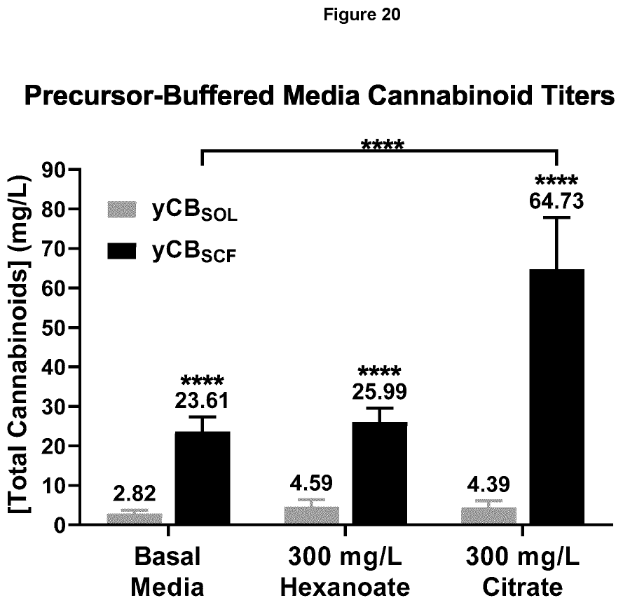

To evaluate the impacts of culture media supplementation with citrate and hexanoate precursors, cannabinoid titers were compared between triplicate yCB_{SOL} and yCB_{SCF} cultures grown in 100 mL YPD media containing 300 mg/L of either buffered citrate (pH 6.0) or hexanoate for 48 hours at 30° C. and 400 RPM in an orbital shaker. All cultures were initially diluted to OD₆₀₀ₙₘ=0.3. Cannabinoid titers for cultures grown in YPD media, citrate-supplemented YPD media, and hexanoate-supplemented YPD media were assessed and analyzed by ANOVA. As shown in FIG. 20, mixed ANOVA detected main effects of strain (F_{1,4}=457.5; p<0.0001) and culture media supplementation (F_{2,8}=312.5; p<0.0001) and a significant strain x culture media supplementation interaction (F_{2,8}=289.6; p<0.0001). Compared to basal media cultures, yCBSCF but not yCBSOL cultures exhibited increased total cannabinoid titers when cultured in media supplemented with 300 mg/L citrate (p<0.0001). Neither yCBSCF nor yCBSOL cultures differed in total cannabinoid titers relative to basal media when cultured in media supplemented with 300 mg/L hexanoate. For all measures, n=3 biological replicates for yCBSCF and yCBSOL cultures. Moreover, relative to yCBSOL cultures, yCBSCF cultures exhibited increased total cannabinoid titers when cultured in basal media (p<0.0001, data also reported in FIG. 19) as well as media supplemented with 300 mg/L citrate (p<0.0001) and hexanoate (p<0.0001).

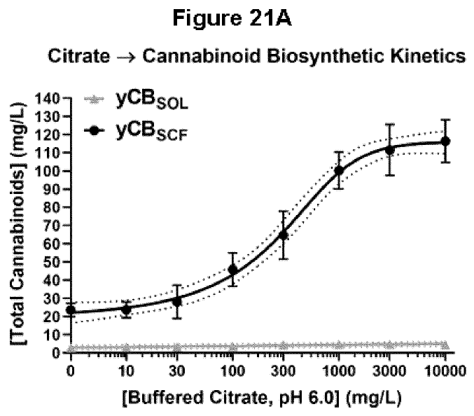

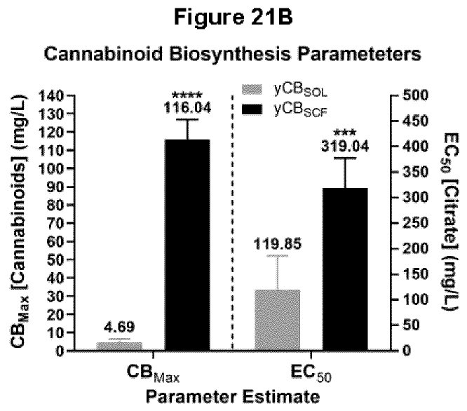

To delineate concentration-response relationships for the supplementation of culture media with citrate, cannabinoid titers were compared between triplicate yCB_{SOL} and yCB_{SCF} cultures grown in 100 mL YPD media containing 0, 10, 30, 100, 300, 1000, 3000, and 10000 mg/L buffered citrate (pH 6.0) for 48 hours at 30° C. and 400 RPM in an orbital shaker. All cultures were initially diluted to OD₆₀₀ₙₘ=0.3. Following quantification, asymmetric sigmoidal (five-parameter) logistic regressions were computed to fit concentration-response curves, from which were derived estimates of the maximal cannabinoid titer (CB_{Max}) and citrate EC₅₀ for cannabinoid biosynthesis in yCB_{SOL} and yCB_{SCF} cultures. Concentration-response curves, CB_{Max} estimates, and citrate EC50 estimates are depicted in FIGS. 21A and 21B. Mixed ANOVA detected main effects of strain (F_{1,8}=69.9; p<0.0001) and parameter (F_{1,8}=66.7; p<0.0001) and a significant strain x parameter interaction (F_{1,8}=5.3; p<0.05) for concentration-response parameter estimates (CBMax and citrate EC50). Compared to yCBSOL cultures, yCBSCF cultures exhibited markedly increased CBMax (p<0.0001) and citrate EC50 (p<0.001) estimates.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A host cell comprising:
(a) a first exogenous nucleic acid encoding a first polypeptide having ATP citrate lyase activity and comprising a first heterologous interaction domain,
(b) a second exogenous nucleic acid encoding a second polypeptide having acetyl-CoA acetyltransferase activity and comprising a second heterologous interaction domain, and
(c) a third exogenous nucleic acid encoding a polypeptide scaffold comprising a peptide ligand for each of said first and second heterologous interaction domains,
wherein each of said first and second heterologous interaction domains is different,
wherein each peptide ligand for each of said first and second heterologous interaction domains is different,
wherein said polypeptide scaffold comprises, in an order extending from amino terminus to carboxy terminus, a first copy of said peptide ligand for said first heterologous interaction domain, a first copy of said peptide ligand for said second heterologous interaction domain, a second copy of said peptide ligand for said second heterologous interaction domain, and a second copy of said peptide ligand for said first heterologous interaction domain.

2. The host cell of claim 1, wherein said host cell is a bacterial or a yeast host cell.

3. The host cell of claim 2, wherein said bacterial cell is selected from the group consisting of Escherichia coli, Bacillus, Brevibacterium, Streptomyces, and Pseudomonas cells.

4. The host cell of claim 2, wherein said yeast cell is selected from the group consisting of Pichia pastoris, Saccharomyces cerevisiae, Yarrowia lipolytica, Kluyveromyces marxianus, and Komagataella phaffii cells.

5. The host cell of claim 1, wherein said host cell is an algae or a plant cell.

6. The host cell of claim 5, wherein said algae is Dunaliella sp., Chlorella variabilis, Euglena mutabilis, or Chlamydomonas reinhardtii cells.

7. The host cell of claim 5, wherein said plant cell is a Cannabis or tobacco cell.

8. The host cell of claim 1, wherein each of said first and second polypeptides is of the formula: enzyme—linker₁—spacer—linker₂—motif₁—linker₃—motif₂, wherein linker₁, linker₂, and linker₃ are the same or different, wherein motif₁ and motif₂ are the same or different, and wherein motif₁ and motif₂ form said heterologous interaction domain.

9. The host cell of claim 8, wherein said scaffold polypeptide comprises a linker between each adjacent peptide ligand.

10. The host cell of claim 9, wherein said scaffold polypeptide is tagged with a MYC tag, FLAG tag, or HA tag.

11. The host cell of claim 9, wherein said linker is a flexible GS-rich sequence flanking a rigid α-helical moiety.

12. The host cell of claim 9, wherein said spacer is the cTPR6 spacer.

13. The host cell of claim 1, wherein a constitutive promoter is operably linked to one or both of said exogenous nucleic acids encoding said polypeptides or to said third exogenous nucleic acid encoding said polypeptide scaffold.

14. The host cell of claim 1, wherein a first constitutive promoter is operably linked to one or both of said exogenous nucleic acids encoding said polypeptides and a second constitutive promoter is operably linked to said third exogenous nucleic acid encoding said polypeptide scaffold.

15. The host cell of claim 14, wherein said constitutive promoter used to express said polypeptide scaffold has weaker constitutive activity level than said constitutive promoter used to express said polypeptides.

16. The host cell of claim 1, wherein each said exogenous nucleic acid comprises an inducible promoter operably linked to the sequence encoding said polypeptide or said polypeptide scaffold.

17. The host cell of claim 16, wherein said promoter is the GAL1-10 promoter.
